(19) United States
(12) Patent Application Publication
(10) Pub. No.: US 2022/0344600 A1
(43) Pub. Date: Oct. 27, 2022

(54) ORGANOMETALLIC COMPOUND, ORGANIC LIGHT-EMITTING DEVICE INCLUDING THE SAME, AND DIAGNOSTIC COMPOSITION INCLUDING THE ORGANOMETALLIC COMPOUND

(71) Applicant: Samsung Electronics Co., Ltd., Suwon-si (KR)

(72) Inventors: Kum Hee Lee, Suwon-si (KR); Seungyeon Kwak, Suwon-si (KR); Juhee Moon, Hwaseong-si (KR); Banglin Lee, Suwon-si (KR); Sunghun Lee, Hwaseong-si (KR); Yong Joo Lee, Suwon-si (KR); Jeoungin Yi, Seoul (KR); Byoungki Choi, Hwaseong-si (KR)

(21) Appl. No.: 17/683,671

(22) Filed: Mar. 1, 2022

(30) Foreign Application Priority Data
Mar. 5, 2021 (KR) 10-2021-0029640

(51) Int. Cl. H01L 51/00; C07F 15/00; C09K 11/06

(52) U.S. Cl. CPC H01L 51/0085; C07F 15/0033; H01L 51/0094; C09K 11/06; C09K 2211/185; C09K 2211/1029; C09K 2211/1048; H01L 51/5012

(57) ABSTRACT

An organometallic compound represented by Formula 1:

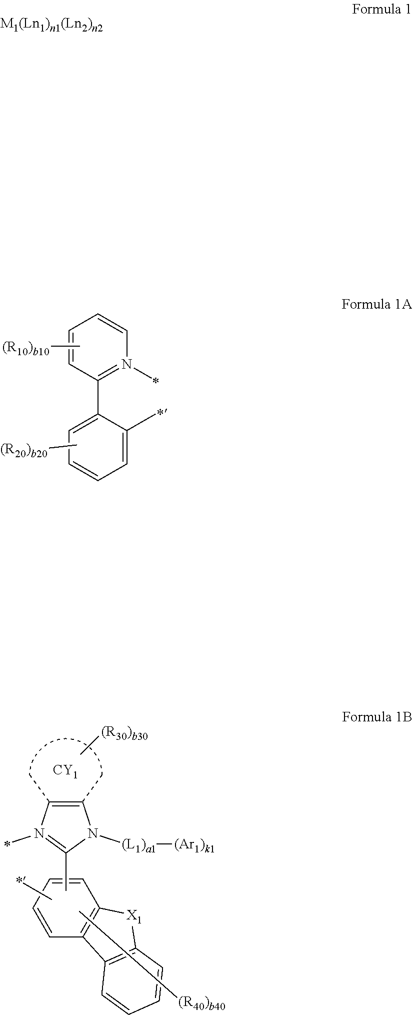

wherein Ln₁ is a ligand represented by Formula 1A, Ln₂ is a ligand represented by Formula 1B, X₁ is O, S, or Se, CY₁ is a C₅-C₃₀ carbocyclic group or a C₁-C₃₀ heterocyclic group, n1 is 1 or 2, n2 is 1, 2, or 3, and the remaining substituents are as defined in the present specification.

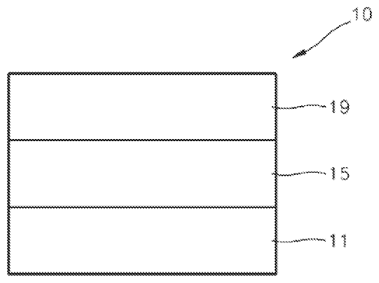

### CROSS-REFERENCE TO RELATED APPLICATION

This application claims priority to Korean Patent Application No. 10-2021-0029640, filed on Mar. 5, 2021 in the Korean Intellectual Property Office, and all benefits accruing therefrom under 35 U.S.C. § 119, the content of which is incorporated by reference herein in its entirety.

### BACKGROUND

### 1. Field

One or more embodiments relate to organometallic compounds, organic light-emitting devices including the same, and diagnostic compositions including the organometallic compounds.

### 2. Description of the Related Art

Organic light-emitting devices (OLEDs) are self-emissive devices, which have improved characteristics in terms of viewing angles, response time, brightness, driving voltage, and response speed, and produce full-color images.

In an example, an organic light-emitting device includes an anode, a cathode, and an organic layer between the anode and the cathode, wherein the organic layer includes an emission layer. A hole transport region may be located between the anode and the emission layer, and an electron transport region may be located between the emission layer and the cathode. Holes provided from the anode may move toward the emission layer through the hole transport region, and electrons provided from the cathode may move toward the emission layer through the electron transport region. The holes and the electrons recombine in the emission layer to produce excitons. These excitons transition from an excited state to a ground state to thereby generate light, for example visible light.

### SUMMARY

One or more embodiments relate to organometallic compounds, organic light-emitting devices including the same, and diagnostic compositions including the organometallic compounds.

Additional aspects will be set forth in part in the description, which follows and, in part, will be apparent from the description, or may be learned by practice of the presented embodiments of the disclosure.

According to one or more embodiments, provided is an organometallic compound represented by Formula 1.

M₁(Ln₁)ₙ₁(Ln₂)ₙ₂ Formula 1

In Formula 1,

M₁ is a transition metal,

Ln₁ is a ligand represented by Formula 1A,

Ln₂ is a ligand represented by Formula 1B,

n1 is 1 or 2, and

n2 is 1, 2, or 3,

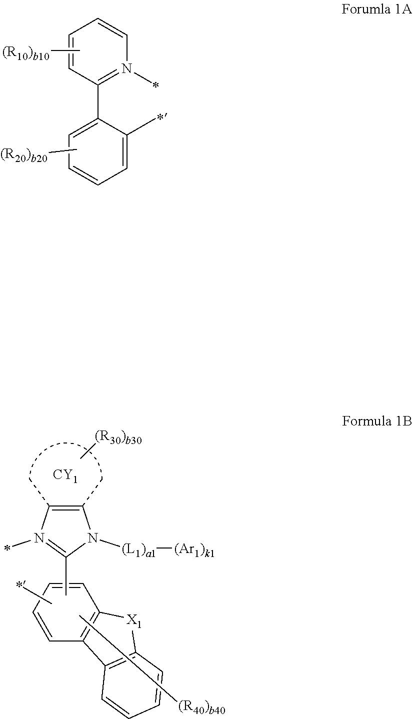

wherein, in Formulae 1A and 1B,

X₁ is O, S, or Se,

CY₁ is a C₅-C₃₀ carbocyclic group or a C₁-C₃₀ heterocyclic group,

L₁ is a substituted or unsubstituted C₃-C₁₀ cycloalkylene group, a substituted or unsubstituted C₁-C₁₀ heterocycloalkylene group, a substituted or unsubstituted C₃-C₁₀ cycloalkenylene group, a substituted or unsubstituted C₂-C₁₀ heterocycloalkenylene group, a substituted or unsubstituted C₆-C₆₀ arylene group, a substituted or unsubstituted C₁-C₆₀ heteroarylene group, a substituted or unsubstituted divalent non-aromatic condensed polycyclic group, or a substituted or unsubstituted divalent non-aromatic condensed heteropolycyclic group,

a1 is 0, 1, 2 or 3,

Ar₁, R₁₀, R₂₀, R₃₀, and R₄₀ may each independently be hydrogen, deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₁-C₆₀ alkylthio group, a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₂-C₁₀ heterocycloalkenyl group, a substituted or unsubstituted C₆-C₆₀ aryl group, a substituted or unsubstituted C₇-C₆₀ alkyl aryl group, a substituted or unsubstituted C₇-C₆₀ aryl alkyl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted C₂-C₆₀ alkyl heteroaryl group, a substituted or unsubstituted C₂-C₆₀ heteroaryl alkyl group, a substituted or unsubstituted C₁-C₆₀ heteroaryloxy group, a substituted or unsubstituted C₁-C₆₀ heteroarylthio group, a substituted or unsubstituted monovalent non-aromatic condensed polycyclic group, a substituted or unsubstituted monovalent non-aromatic condensed heteropolycyclic group, —N(Q₁)(Q₂), —Si(Q₃)(Q₄)(Q₅), —Ge(Q₃)(Q₄)(Q₅), —B(Q₆)(Q₇), —C(═O)(Q₆), —S(═O)₂(Q₆), —P(═O)(Q₈)(Q₉), or —P(Q₈)(Q₉),

k1 is 1, 2, 3, 4, 5, 6, 7, or 8,

b10 and b20 are each independently 1, 2, 3, or 4,

b30 is 1, 2, 3, 4, 5, 6, 7, or 8,

b40 is 1, 2, 3, 4, 5, or 6,

wherein the organometallic compound comprises at least one —F,

* and *′ each indicate a binding site to M₁,

at least one substituent of the substituted C₁-C₆₀ alkyl group, the substituted C₂-C₆₀ alkenyl group, the substituted C₂-C₆₀ alkynyl group, the substituted C₁-C₆₀ alkoxy group, the substituted C₁-C₆₀ alkylthio group, the substituted C₃-C₁₀ cycloalkyl group, the substituted C₁₁-C₁₀ heterocycloalkyl group, the substituted C₃-C₁₀ cycloalkenyl group, the substituted C₁-C₁₀ heterocycloalkenyl group, the substituted C₆-C₆₀ aryl group, the substituted C₇-C₆₀ alkyl aryl group, the substituted C₇-C₆₀ aryl alkyl group, the substituted C₆-C₆₀ aryloxy group, the substituted C₆-C₆₀ arylthio group, the substituted C₁-C₆₀ heteroaryl group, the substituted C₂-C₆₀ alkyl heteroaryl group, the substituted C₂-C₆₀ heteroaryl alkyl group, the substituted C₁-C₆₀ heteroaryloxy group, the substituted C₁-C₆₀ heteroarylthio group, the substituted monovalent non-aromatic condensed polycyclic group, and the substituted monovalent non-aromatic condensed heteropolycyclic group is:

deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkoxy group, or a C₁-C₆₀ alkylthio group,

a C₁-C₆₀ alkyl group, a C₂-C₆₀ alkenyl group, a C₂-C₆₀ alkynyl group, or a C₁-C₆₀ alkoxy group, each substituted with deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₃-C₁₀ cycloalkyl group, a C₁-C₁₀ heterocycloalkyl group, a C₃-C₁₀ cycloalkenyl group, a C₂-C₁₀ heterocycloalkenyl group, a C₆-C₆₀ aryl group, a C₇-C₆₀ alky aryl group, a C₇-C₆₀ aryl alkyl group, a C₆-C₆₀ aryloxy group, a C₆-C₆₀ arylthio group, a C₁-C₆₀ heteroaryl group, a C₂-C₆₀ alkyl heteroaryl group, a C₂-C₆₀ heteroaryl alkyl group, a C₁-C₆₀ heteroaryloxy group, a C₁-C₆₀ heteroarylthio group, a monovalent non-aromatic condensed polycyclic group, a monovalent non-aromatic condensed heteropolycyclic group, —N(Q₁₁)(Q₁₂), —Si(Q₁₃)(Q₁₄)(Q₁₅), —Ge(Q₁₃)(Q₁₄)(Q₁₅), —B(Q₁₆)(Q₁₇), —C(═O)(Q₁₆), —S(═O)₂(Q₁₆), —P(═O)(Q₁₈)(Q₁₉), —P(═O)(Q₁₈)(Q₁₉), or a combination thereof,

a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₇-C₆₀ alky aryl group, a C₆-C₆₀ aryloxy group, a C₆-C₆₀ arylthio group, a C₁-C₆₀ heteroaryl group, a C₂-C₆₀ alkyl heteroaryl group, a C₁-C₆₀ heteroaryloxy group, a C₁-C₆₀ heteroarylthio group, a monovalent non-aromatic condensed polycyclic group, or a monovalent non-aromatic condensed heteropolycyclic group,

a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₇-C₆₀ alky aryl group, a C₆-C₆₀ aryloxy group, a C₆-C₆₀ arylthio group, a C₁-C₆₀ heteroaryl group, a C₂-C₆₀ alkyl heteroaryl group, a C₁-C₆₀ heteroaryloxy group, a C₁-C₆₀ heteroarylthio group, a monovalent non-aromatic condensed polycyclic group, or a monovalent non-aromatic condensed heteropolycyclic group, each substituted with deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkoxy group, a C₁-C₆₀ alkylthio group, a C₃-C₁₀ cycloalkyl group, a C₁-C₁₀ heterocycloalkyl group, a C₃-C₁₀ cycloalkenyl group, a C₂-C₁₀ heterocycloalkenyl group, a C₆-C₆₀ aryl group, a C₇-C₆₀ alky aryl group, a C₇-C₆₀ aryl alkyl group, a C₆-C₆₀ aryloxy group, a C₆-C₆₀ arylthio group, a C₁-C₆₀ heteroaryl group, a C₂-C₆₀ alkyl heteroaryl group, a C₂-C₆₀ heteroaryl alkyl group, a C₁-C₆₀ heteroaryloxy group, a C₁-C₆₀ heteroarylthio group, a monovalent non-aromatic condensed polycyclic group, a monovalent non-aromatic condensed heteropolycyclic group, —N(Q₂₁)(Q₂₂), —Si(Q₂₃)(Q₂₄)(Q₂₅), —Ge(Q₂₃)(Q₂₄)(Q₂₅), —B(Q₂₆)(Q₂₇), —C(═O)(Q₂₆), —S(═O)₂(Q₂₆), —P(═O)(Q₂₈)(Q₂₉), —P(Q₂₈)(Q₂₉), or a combination thereof, or

—N(Q₃₁)(Q₃₂), —Si(Q₃₃)(Q₃₄)(Q₃₅), —Ge(Q₃₃)(Q₃₄)(Q₃₅), —B(Q₃₆)(Q₃₇), —C(═O)(Q₃₆), —S(═O)₂(Q₃₆), —P(═O)(Q₃₈)(Q₃₉), —P(Q₃₈)(Q₃₉),

wherein Q₁ to Q₉, Q₁₁ to Q₁₉, Q₂₁ to Q₂₉, and Q₃₁ to Q₃₉ are each independently hydrogen, deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₁-C₆₀ alkylthio group, a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₁-C₁₀ heterocycloalkenyl group, a substituted or unsubstituted C₆-C₆₀ aryl group, a substituted or unsubstituted C₇-C₆₀ alkyl aryl group, a substituted or unsubstituted C₇-C₆₀ aryl alkyl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted C₂-C₆₀ alkyl heteroaryl group, a substituted or unsubstituted C₂-C₆₀ heteroaryl alkyl group, a C₁-C₆₀ heteroaryloxy group, a C₁-C₆₀ heteroarylthio group, a substituted or unsubstituted monovalent non-aromatic condensed polycyclic group, or a substituted or unsubstituted monovalent non-aromatic condensed heteropolycyclic group.

Another aspect provides an organic light-emitting device including a first electrode, a second electrode, and an organic layer including an emission layer and located between the first electrode and the second electrode, wherein the organic layer includes at least one of the organometallic compounds.

The organometallic compound may be included in the emission layer of the organic layer, and the organometallic compound included in the emission layer may act as a dopant.

Another aspect provides a diagnostic composition including at least one organometallic compound represented by Formula 1.

### BRIEF DESCRIPTION OF THE DRAWING

The above and other aspects, features, and advantages of certain embodiments of the disclosure will be more apparent from the following description taken in conjunction with the accompanying drawing, in which

FIGURE shows a schematic cross-sectional view of an organic light-emitting device according to one or more embodiments.

### DETAILED DESCRIPTION

Reference will now be made in further detail to exemplary embodiments, examples of which are illustrated in the accompanying drawing, wherein like reference numerals refer to like elements throughout the specification. In this regard, the present exemplary embodiments may have different forms and should not be construed as being limited to the descriptions set forth herein. Accordingly, the exemplary embodiments are merely described below, by referring to the FIGURE, to explain various aspects. As used herein, the term “and/or” includes any and all combinations of one or more of the associated listed items. Expressions such as “at least one of,” when preceding a list of elements, modify the entire list of elements and do not modify the individual elements of the list.

The terminology used herein is for the purpose of describing one or more exemplary embodiments only and is not intended to be limiting. As used herein, the singular forms “a,” “an,” and “the” are intended to include the plural forms as well, unless the context clearly indicates otherwise. The term “or” means “and/or.” It will be further understood that the terms “comprises” and/or “comprising,” or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof.

It will be understood that, although the terms first, second, third etc. may be used herein to describe various elements, components, regions, layers, and/or sections, these elements, components, regions, layers, and/or sections should not be limited by these terms. These terms are only used to distinguish one element, component, region, layer, or section from another element, component, region, layer, or section. Thus, a first element, component, region, layer, or section discussed below could be termed a second element, component, region, layer, or section without departing from the teachings of the present embodiments.

Exemplary embodiments are described herein with reference to cross section illustrations that are schematic illustrations of idealized embodiments. As such, variations from the shapes of the illustrations as a result, for example, of manufacturing techniques and/or tolerances, are to be expected. Thus, embodiments described herein should not be construed as limited to the particular shapes of regions as illustrated herein but are to include deviations in shapes that result, for example, from manufacturing. For example, a region illustrated or described as flat may, typically, have rough and/or nonlinear features. Moreover, sharp angles that are illustrated may be rounded. Thus, the regions illustrated in the figures are schematic in nature and their shapes are not intended to illustrate the precise shape of a region and are not intended to limit the scope of the present claims.

It will be understood that when an element is referred to as being “on” another element, it can be directly in contact with the other element or intervening elements may be present therebetween. In contrast, when an element is referred to as being “directly on” another element, there are no intervening elements present.

Unless otherwise defined, all terms (including technical and scientific terms) used herein have the same meaning as commonly understood by one of ordinary skill in the art to which this general inventive concept belongs. It will be further understood that terms, such as those defined in commonly used dictionaries, should be interpreted as having a meaning that is consistent with their meaning in the context of the relevant art and the present disclosure, and will not be interpreted in an idealized or overly formal sense unless expressly so defined herein.

“About” or “approximately” as used herein is inclusive of the stated value and means within an acceptable range of deviation for the particular value as determined by one of ordinary skill in the art, considering the measurement in question and the error associated with measurement of the particular quantity (i.e., the limitations of the measurement system). For example, “about” can mean within one or more standard deviations, or within ±30%, 20%, 10%, 5% of the stated value.

An aspect of the present disclosure provides an organometallic compound represented by Formula 1:

M₁(Ln₁)ₙ₁(Ln₂)ₙ₂ Formula 1

M₁ in Formula 1 is a transition metal.

For example, M₁ may be a Period 1 transition metal of the Periodic Table of Elements, a Period 2 transition metal of the Periodic Table of Elements, or a Period 3 transition metal of the Periodic Table of Elements.

In one or more embodiments, M₁ may be iridium (Ir), platinum (Pt), osmium (Os), titanium (Ti), zirconium (Zr), hafnium (Hf), europium (Eu), terbium (Tb), thulium (Tm)), or rhodium (Rh).

In one or more embodiments, M₁ may be Ir, Pt, Os, or Rh.

In one or more embodiments, M₁ may be Ir.

In Formula 1, n1 is 1 or 2, and n2 is 1, 2, or 3.

In one or more embodiments, the sum of n1 and n2 may be 2 or 3.

In one or more embodiments, M₁ may be Ir and the sum of n1 and n2 may be 3.

In one or more embodiments, M₁ may be Pt, and the sum of n1 and n2 may be 2.

Ln₁ in Formula 1 is a ligand represented by Formula 1A, and

Ln₂ may be a ligand represented by Formula 11B:

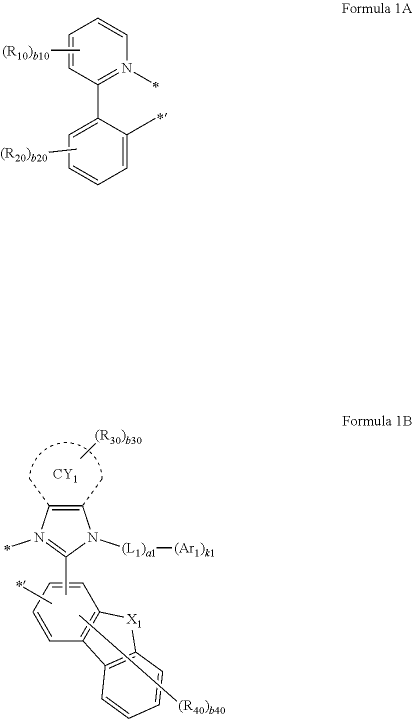

X₁ in Formulae 1A and 1B is O, S, or Se.

The organometallic compound includes at least one —F.

In one or more embodiments, the organometallic compound may include 1 to 4 of —F.

CY₁ in Formulae 1A and 1B may be a C₅-C₃₀ carbocyclic group or a C₁-C₃₀ heterocyclic group.

In one or more embodiments, ring CY₁ may be i) a first ring, ii) a second ring, iii) a condensed cyclic group in which two or more first rings are condensed with each other, iv) a condensed cyclic group in which two or more second rings are condensed with each other, or v) a condensed cyclic group in which at least one first ring is condensed with at least one second ring,

the first ring may be a cyclopentane group, a cyclopentadiene group, a furan group, a thiophene group, a pyrrole group, a silole group, an indene group, a benzofuran group, a benzothiophene group, an indole group, a benzosilole group, an oxazole group, an isoxazole group, an oxadiazole group, an isoxadiazole group, an oxatriazole group, an isoxatriazole group, a thiazole group, an isothiazole group, a thiadiazole group, an isothiadiazole group, a thiatriazole group, an isothiatriazole group, a pyrazole group, an imidazole group, a triazole group, a tetrazole group, an azasilole group, a diazasilole group, or a triazasilole group, and

the second ring may be an adamantane group, a norbornane group, a norbornene group, a cyclohexane group, a cyclohexene group, a benzene group, a pyridine group, a pyrimidine group, a pyrazine group, a pyridazine group, or a triazine group.

In one or more embodiments, CY₁ may be a cyclopentane group, a cyclohexane group, a cycloheptane group, a cyclopentene group, a cyclohexene group, a cycloheptene group, a benzene group, a naphthalene group, an anthracene group, a phenanthrene group, a triphenylene group, a pyrene group, a chrysene group, a cyclopentadiene group, a 1,2,3,4-tetrahydronaphthalene group, a thiophene group, a furan group, an indole group, a benzoborole group, a benzophosphole group, an indene group, a benzosilole group, a benzogermole group, a benzothiophene group, a benzoselenophene group, a benzofuran group, a carbazole group, a dibenzoborole group, a dibenzophosphole group, a fluorene group, a dibenzosilole group, a dibenzogermole group, a dibenzothiophene group, a dibenzoselenophene group, a dibenzofuran group, a dibenzothiophene 5-oxide group, a 9H-fluoren-9-one group, a dibenzothiophene 5,5-dioxide group, an azaindole group, an azabenzoborole group, an azabenzophosphole group, an azaindene group, an azabenzosilole group, an azabenzogermole group, an azabenzothiophene group, an azabenzoselenophene group, an azabenzofuran group, an azacarbazole group, an azadibenzoborole group, an azadibenzophosphole group, an azafluorene group, an azadibenzosilole group, an azadibenzogermole group, an azadibenzothiophene group, an azadibenzoselenophene group, an azadibenzofuran group, an azadibenzothiophene 5-oxide group, an aza-9H-fluoren-9-one group, an azadibenzothiophene 5,5-dioxide group, a pyridine group, a pyrimidine group, a pyrazine group, a pyridazine group, a triazine group, a quinoline group, an isoquinoline group, a quinoxaline group, a quinazoline group, a phenanthroline group, a pyrrole group, a pyrazole group, an imidazole group, a triazole group, an oxazole group, an isooxazole group, a thiazole group, an isothiazole group, an oxadiazole group, a thiadiazole group, a benzopyrazole group, a benzimidazole group, a benzoxazole group, a benzothiazole group, a benzoxadiazole group, a benzothiadiazole group, a 5,6,7,8-tetrahydroisoquinoline group, or a 5,6,7,8-tetrahydroquinoline group.

In one or more embodiments, CY₁ may be a benzene group, a naphthalene group, a 1,2,3,4-tetrahydronaphthalene group, a phenanthrene group, a pyridine group, a pyrimidine group, a pyrazine group, a triazine group, a quinoline group, an isoquinoline group, a quinoxaline group, a quinazoline group, a phenanthroline group, a benzofuran group, a benzothiophene group, a fluorene group, a carbazole group, a dibenzofuran group, a dibenzothiophene group, a dibenzosilole group, an azafluorene group, an azacarbazole group, an azadibenzofuran group, an azadibenzothiophene group, or an azadibenzosilole group.

In one or more embodiments, CY₁ may be a benzene group or a naphthalene group.

L₁ in Formula 1B may be a substituted or unsubstituted C₃-C₁₀ cycloalkylene group, a substituted or unsubstituted C₁-C₁₀ heterocycloalkylene group, a substituted or unsubstituted C₃-C₁₀ cycloalkenylene group, a substituted or unsubstituted C₂-C₁₀ heterocycloalkenylene group, a substituted or unsubstituted C₆-C₆₀ arylene group, a substituted or unsubstituted C₁-C₆₀ heteroarylene group, a substituted or unsubstituted divalent non-aromatic condensed polycyclic group, or a substituted or unsubstituted divalent non-aromatic condensed heteropolycyclic group.

In one or more embodiments, L₁ may be: a phenylene group, a naphthylene group, a fluorenylene group, a spiro-bifluorenylene group, a benzofluorenylene group, a dibenzofluorenylene group, a phenanthrenylene group, an anthracenylene group, a fluoranthenylene group, a triphenylenylene group, a pyrenylene group, a chrysenylene group, a perylenylene group, a thiophenylene group, a furanylene group, a carbazolylene group, an indolylene group, an isoindolylene group, a benzofuranylene group, a benzothiophenylene group, a dibenzofuranylene group, a dibenzothiophenylene group, a benzocarbazolylene group, a dibenzocarbazolylene group, a dibenzosilolylene group, a pyridinylene group, an imidazolylene group, a pyrazolylene group, a thiazolylene group, an isothiazolylene group, an oxazolylene group, an isoxazolylene group, a thiadiazolylene group, an oxadiazolylene group, a pyrazinylene group, a pyrimidinylene group, a pyridazinylene group, a triazinylene group, a quinolinylene group, an isoquinolinylene group, a benzoquinolinylene group, a phthalazinylene group, a naphthyridinylene group, a quinoxalinylene group, a quinazolinylene group, a cinnolinylene group, a phenanthridinylene group, an acridinylene group, a phenanthrolinylene group, a phenazinylene group, a benzimidazolylene group, an isobenzothiazolylene group, a benzoxazolylene group, an isobenzoxazolylene group, a triazolylene group, a tetrazolylene group, an imidazopyridinylene group, an imidazopyrimidinylene group, or an azacarbazolylene group; or

a phenylene group, a naphthylene group, a fluorenylene group, a spiro-bifluorenylene group, a benzofluorenylene group, a dibenzofluorenylene group, a phenanthrenylene group, an anthracenylene group, a fluoranthenylene group, a triphenylenylene group, a pyrenylene group, a chrysenylene group, a perylenylene group, a thiophenylene group, a furanylene group, a carbazolylene group, an indolylene group, an isoindolylene group, a benzofuranylene group, a benzothiophenylene group, a dibenzofuranylene group, a dibenzothiophenylene group, a benzocarbazolylene group, a dibenzocarbazolylene group, a dibenzosilolylene group, a pyridinylene group, an imidazolylene group, a pyrazolylene group, a thiazolylene group, an isothiazolylene group, an oxazolylene group, an isoxazolylene group, a thiadiazolylene group, an oxadiazolylene group, a pyrazinylene group, a pyrimidinylene group, a pyridazinylene group, a triazinylene group, a quinolinylene group, an isoquinolinylene group, a benzoquinolinylene group, a phthalazinylene group, a naphthyridinylene group, a quinoxalinylene group, a quinazolinylene group, a cinnolinylene group, a phenanthridinylene group, an acridinylene group, a phenanthrolinylene group, a phenazinylene group, a benzimidazolylene group, a benzoisothiazolylene group, a benzoxazolylene group, an isobenzoxazolylene group, a triazolylene group, a tetrazolylene group, an imidazopyridinylene group, an imidazopyrimidinylene group, or an azacarbazolylene group, each substituted with deuterium, —F, —Cl, —Br, —I, —CD₃, —CD₂H, —CDH₂, —CF₃, —CF₂H, —CFH₂, a hydroxyl group, a cyano group, a nitro group, an amidino group, a hydrazino group, a hydrazono group, a C₁-C₂₀ alkyl group, a C₁-C₂₀ alkoxy group, a phenyl group, a biphenyl group, a terphenyl group, a naphthyl group, —Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), —P(═O)(Q₃₁)(Q₃₂), P(Q₃₁)(Q₃₂), or a combination thereof.

In one or more embodiments, L₁ may be a phenylene group, a naphthylene group, a fluorenylene group, a spiro-bifluorenylene group, a benzofluorenylene group, a phenanthrenylene group, an anthracenylene group, a carbazolylene group, a dibenzofuranylene group, a dibenzothiophenylene group, a benzocarbazolylene group, a dibenzocarbazolylene group, or dibenzosilolylene group; or

a phenylene group, a naphthylene group, a fluorenylene group, a spiro-bifluorenylene group, a benzofluorenylene group, a phenanthrenylene group, an anthracenylene group, a carbazolylene group, a dibenzofuranylene group, a dibenzothiophenylene group, a benzocarbazolylene group, a dibenzocarbazolylene group, or dibenzosilolylene group, each substituted with deuterium, —F, —CI, —Br, —I, —CD₃, —CD₂H, —CDH₂, —CF₃, —CF₂H, —CFH₂, a hydroxyl group, a cyano group, a nitro group, an amidino group, a hydrazino group, a hydrazono group, a C₁-C₂₀ alkyl group, a C₁-C₂₀ alkoxy group, a phenyl group, a biphenyl group, a terphenyl group, a naphthyl group, —Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), —P(═O)(Q₃₁)(Q₃₂), P(Q₃₁)(Q₃₂), or a combination thereof,

a1 in Formula 1B may be 0, 1, 2, or 3.

For example, a1 may be 0, 1, or 2. For example, a1 may be 0 or 1.

Ar₁, R₁₀, R₂₀, R₃₀, and R₄₀ in Formulae 1A and 1B are each independently hydrogen, deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₁-C₆₀ alkylthio group, a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₂-C₁₀ heterocycloalkenyl group, a substituted or unsubstituted C₆-C₆₀ aryl group, a substituted or unsubstituted C₇-C₆₀ alkyl aryl group, a substituted or unsubstituted C₇-C₆₀ aryl alkyl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted C₁-C₆₀ heteroaryloxy group, a substituted or unsubstituted C₁-C₆₀ heteroarylthio group, a substituted or unsubstituted monovalent non-aromatic condensed polycyclic group, a substituted or unsubstituted monovalent non-aromatic condensed heteropolycyclic group, —N(Q₁)(Q₂), —Si(Q₃)(Q₄)(Q₅), —Ge(Q₃)(Q₄)(Q₅), —B(Q₆)(Q₇), —C(═O)(Q₆), —S(═O)₂(Q₆), —P(═O)(Q₈)(Q₉), or —P(Q₈)(Q₉).

k1 in Formula 1B is 1, 2, 3, 4, 5, 6, 7, or 8.

In one or more embodiments, k1 may be 1, 2, 3, 4, or 5.

In one or more embodiments, k1 may be 1, 2, or 3.

b10 and b20 in Formula 1A are each independently 1, 2, 3, or 4.

In one or more embodiments, b10 and b20 may each independently be 1, 2, or 3.

In one or more embodiments, b10 and b20 may each independently be 1 or 2.

b30 in Formula 1B is 1, 2, 3, 4, 5, 6, 7, or 8.

In one or more embodiments, b30 may be 1, 2, 3, 4, 5, or 6.

In one or more embodiments, b30 may be 1, 2, 3, or 4.

b40 in Formula 1B may be 1, 2, 3, 4, 5, or 6.

In one or more embodiments, b40 may be 1, 2, or 3.

In one or more embodiments, b40 may be 1 or 2.

In one or more embodiments, b40 may be 1.

In one or more embodiments, Ar₁, R₁₀, R₂₀, R₃₀, and R₄₀ may each independently be: hydrogen, deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, —SF₅, a C₁-C₂₀ alkyl group, or a C₁-C₂₀ alkoxy group; a C₁-C₂₀ alkyl group or a C₁-C₂₀ alkoxy group, each substituted with deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₁₀ alkyl group, a cyclopentyl group, a cyclohexyl group, a cycloheptyl group, a cyclooctyl group, an adamantanyl group, a norbornanyl group, a norbornenyl group, a cyclopentenyl group, a cyclohexenyl group, a cycloheptenyl group, a phenyl group, a naphthyl group, a pyridinyl group, a pyrimidinyl group, or a combination thereof;

a cyclopentyl group, a cyclohexyl group, a cycloheptyl group, a cyclooctyl group, an adamantanyl group, a norbornanyl group, a norbornenyl group, a cyclopentenyl group, a cyclohexenyl group, a cycloheptenyl group, a phenyl group, a naphthyl group, a fluorenyl group, a phenanthrenyl group, an anthracenyl group, a fluoranthenyl group, a triphenylenyl group, a pyrenyl group, a chrysenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a quinoxalinyl group, a quinazolinyl group, a cinnolinyl group, a carbazolyl group, a phenanthrolinyl group, a benzimidazolyl group, a benzofuranyl group, a benzothiophenyl group, an isobenzothiazolyl group, a benzoxazolyl group, an isobenzoxazolyl group, a triazolyl group, a tetrazolyl group, an oxadiazolyl group, a triazinyl group, a dibenzofuranyl group, a dibenzothiophenyl group, a benzocarbazolyl group, a dibenzocarbazolyl group, an imidazopyridinyl group, or an imidazopyrimidinyl group;

a cyclopentyl group, a cyclohexyl group, a cycloheptyl group, a cyclooctyl group, an adamantanyl group, a norbornanyl group, a norbornenyl group, a cyclopentenyl group, a cyclohexenyl group, a cycloheptenyl group, a phenyl group, a naphthyl group, a fluorenyl group, a phenanthrenyl group, an anthracenyl group, a fluoranthenyl group, a triphenylenyl group, a pyrenyl group, a chrysenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a quinoxalinyl group, a quinazolinyl group, a cinnolinyl group, a carbazolyl group, a phenanthrolinyl group, a benzimidazolyl group, a benzofuranyl group, a benzothiophenyl group, an isobenzothiazolyl group, a benzoxazolyl group, an isobenzoxazolyl group, a triazolyl group, a tetrazolyl group, an oxadiazolyl group, a triazinyl group, a dibenzofuranyl group, a dibenzothiophenyl group, a benzocarbazolyl group, a dibenzocarbazolyl group, an imidazopyridinyl group, or an imidazopyrimidinyl group, each substituted with deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₂₀ alkyl group, a C₁-C₂₀ alkoxy group, a cyclopentyl group, a cyclohexyl group, a cycloheptyl group, a cyclooctyl group, an adamantanyl group, a norbornanyl group, a norbornenyl group, a cyclopentenyl group, a cyclohexenyl group, a cycloheptenyl group, a phenyl group, a naphthyl group, a fluorenyl group, a phenanthrenyl group, an anthracenyl group, a fluoranthenyl group, a triphenylenyl group, a pyrenyl group, a chrysenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a quinoxalinyl group, a quinazolinyl group, a cinnolinyl group, a carbazolyl group, a phenanthrolinyl group, a benzimidazolyl group, a benzofuranyl group, a benzothiophenyl group, an isobenzothiazolyl group, a benzoxazolyl group, an isobenzoxazolyl group, a triazolyl group, a tetrazolyl group, an oxadiazolyl group, a triazinyl group, a dibenzofuranyl group, a dibenzothiophenyl group, a benzocarbazolyl group, a dibenzocarbazolyl group, an imidazopyridinyl group, an imidazopyrimidinyl group, or a combination thereof, or

—N(Q₁)(Q₂), —Si(Q₃)(Q₄)(Q₅), —Ge(Q₃)(Q₄)(Q₅), —B(Q₆)(Q₇), —C(═O)(Q₆), —S(═O)₂(Q₆), —P(═O)(Q₈)(Q₉), or —P(Q₈)(Q₉).

* and *′ in Formulae 1A and 1B each indicate a binding site to a neighboring atom.

In one or more embodiments, Ar₁, R₁₀, R₂₀, R₃₀, and R₄₀ may each independently be: hydrogen, deuterium, —F, —Cl, —Br, —I, —CD₃, —CD₂H, —CDH₂, —CF₃, —CF₂H, —CFH₂, a C₁-C₆₀ alkyl group, a C₂-C₆₀ alkenyl group, a C₂-C₆₀ alkynyl group, or a C₁-C₆₀ alkoxy group; or

a group represented by one of Formulae 9-1 to 9-67, 9-201 to 9-244, 10-1 to 10-154, and 10-201 to 10-350:

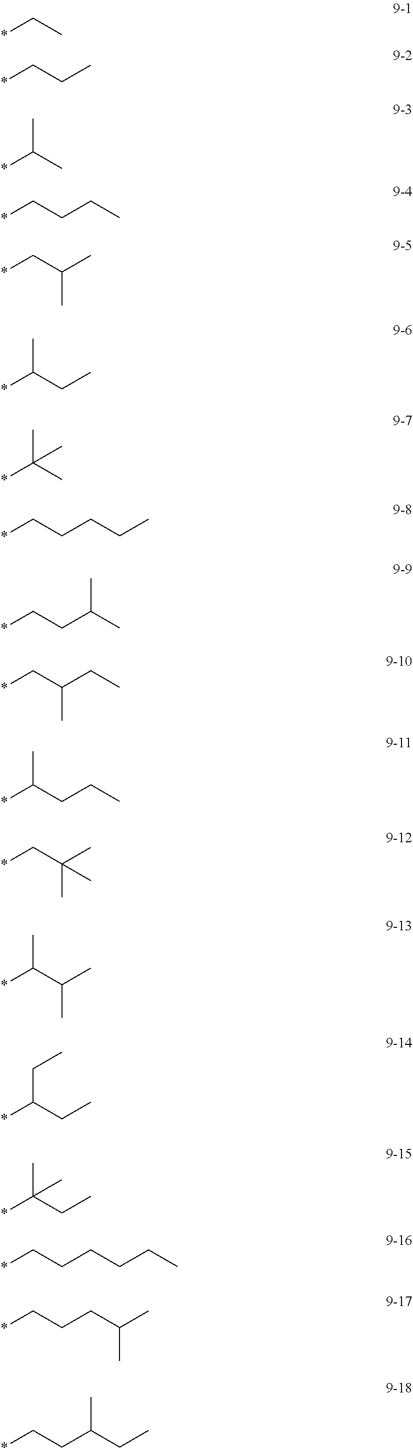

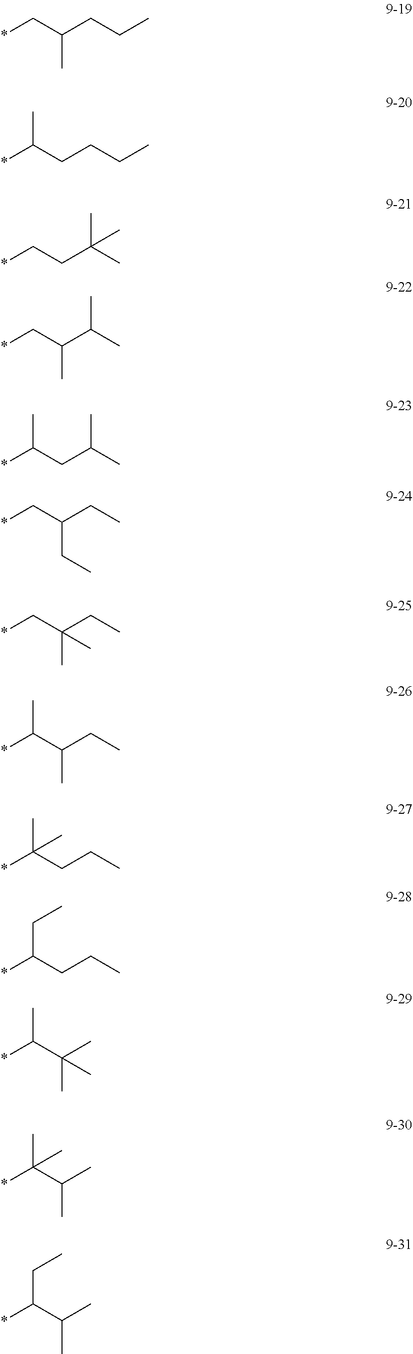

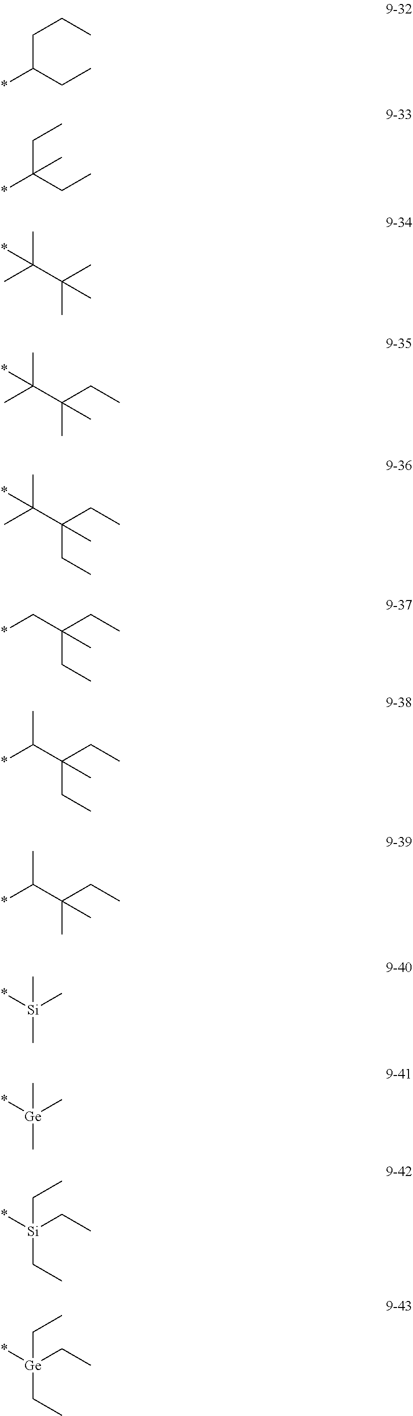

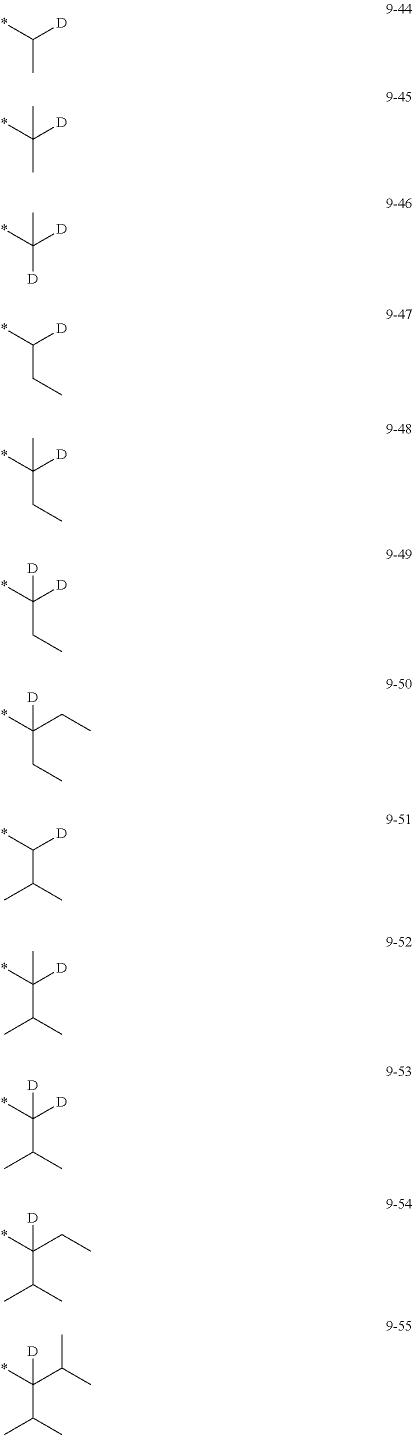

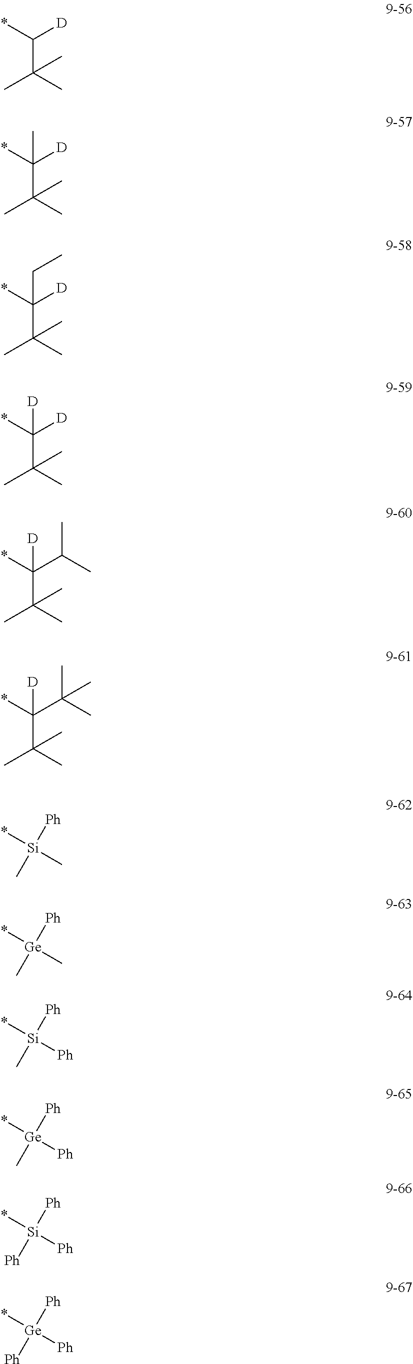

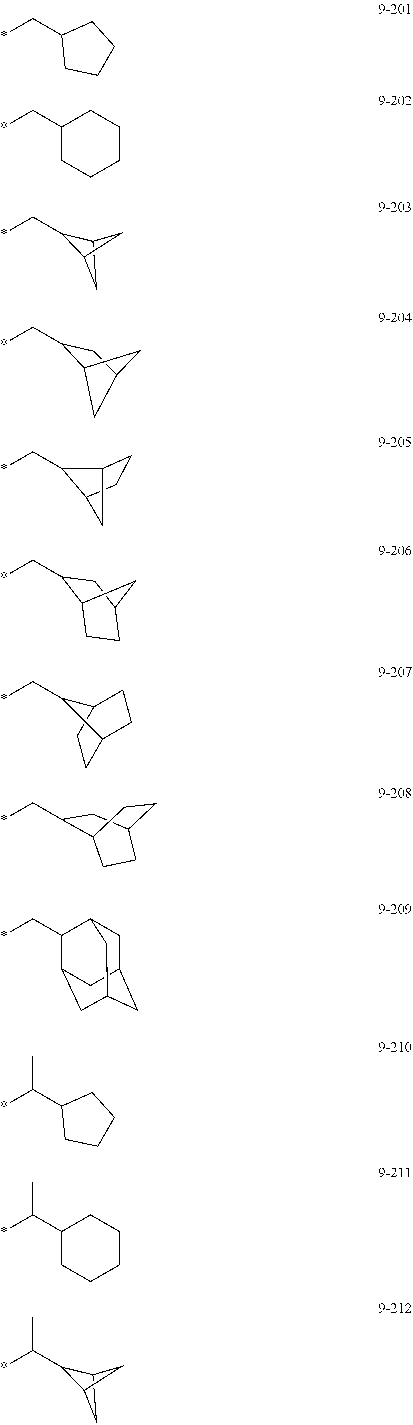

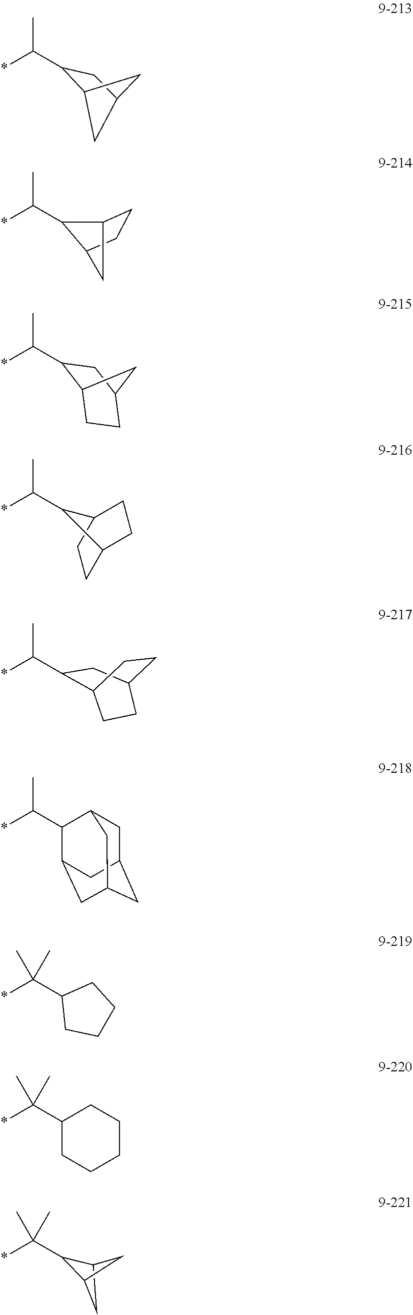

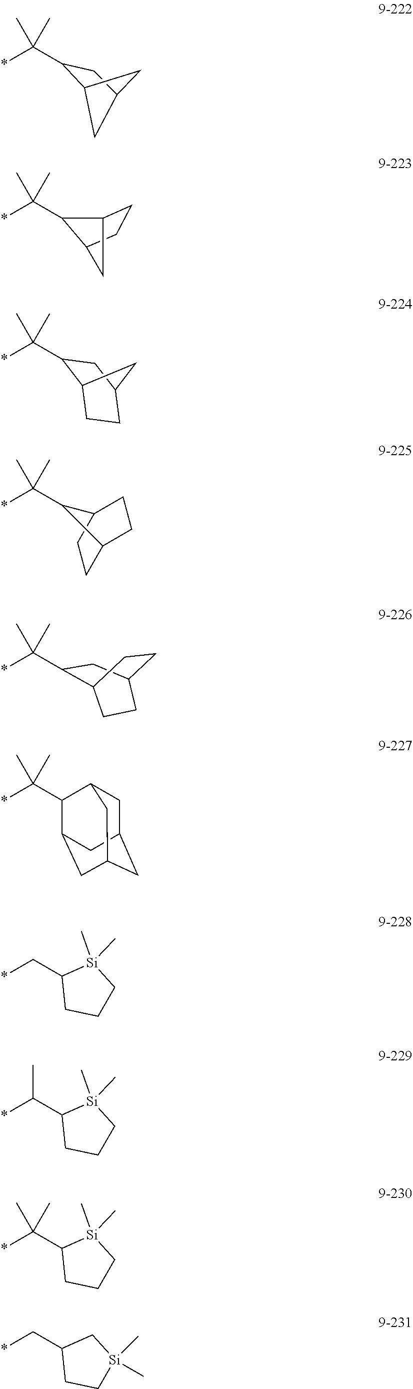

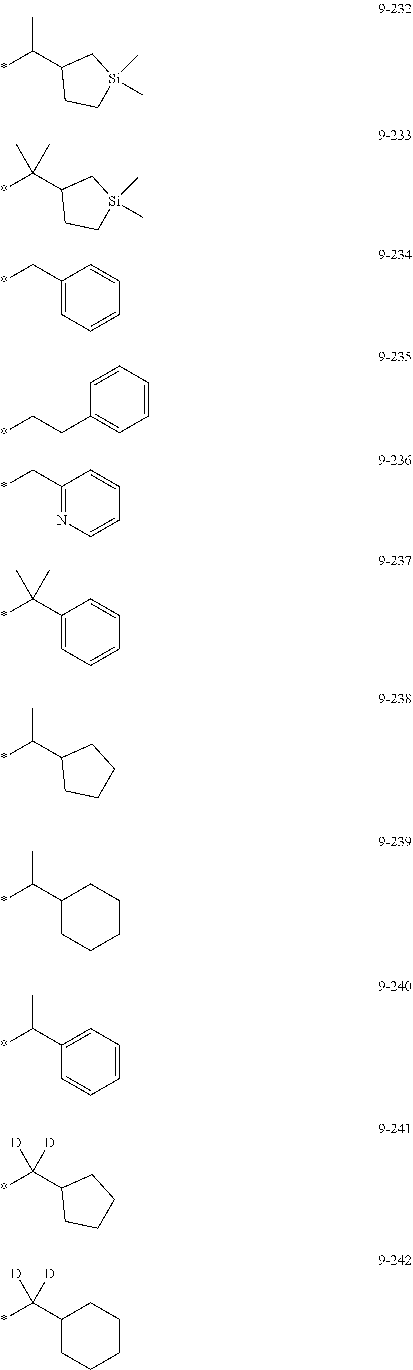

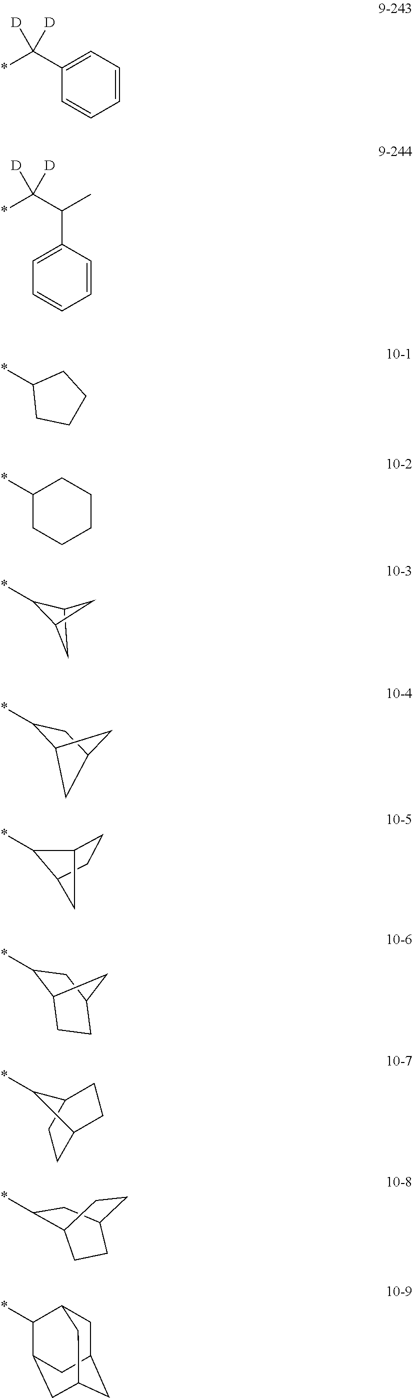

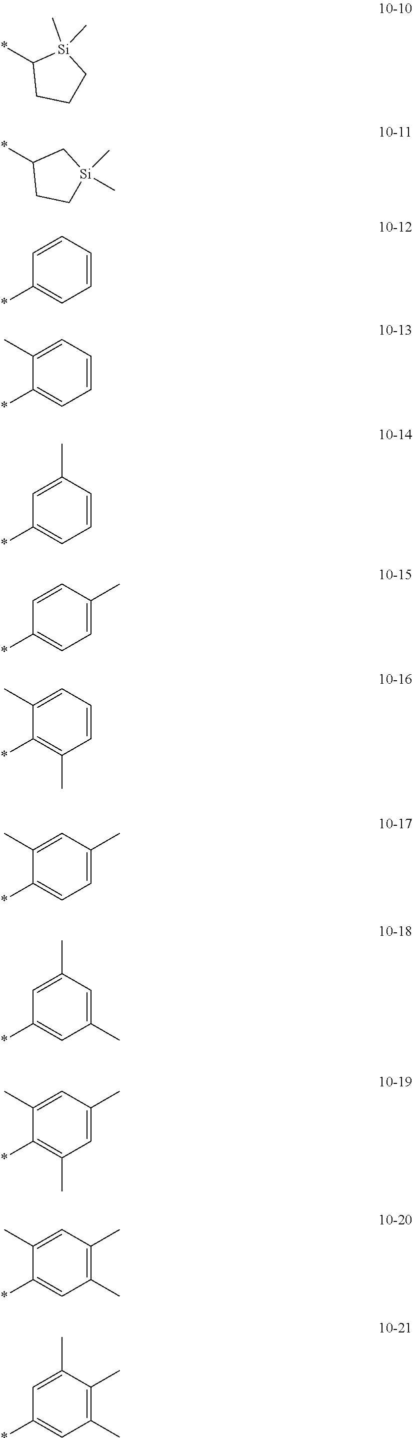

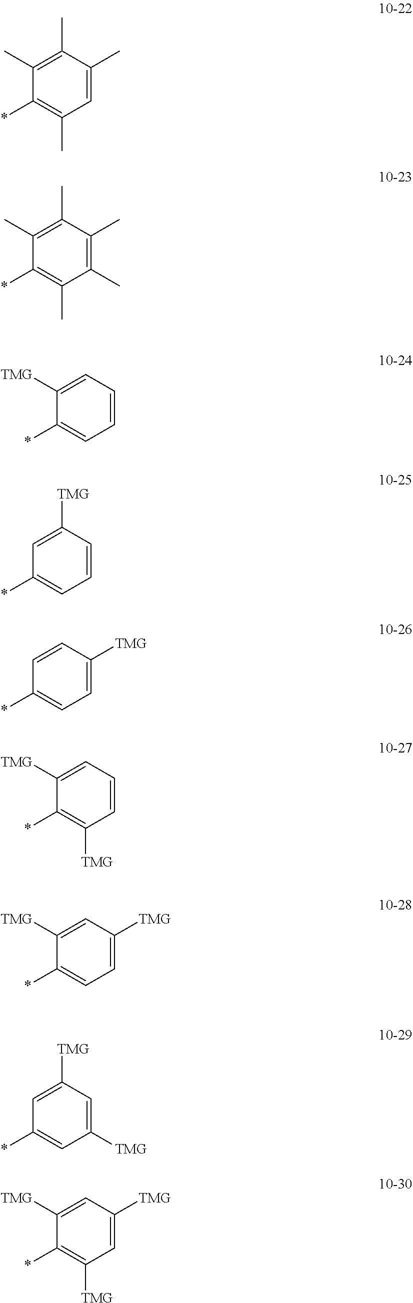

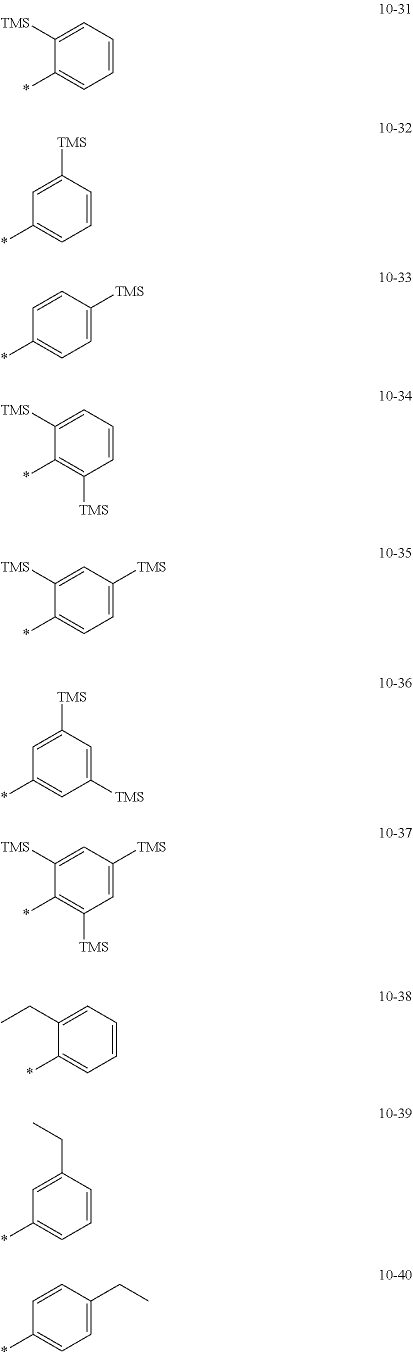

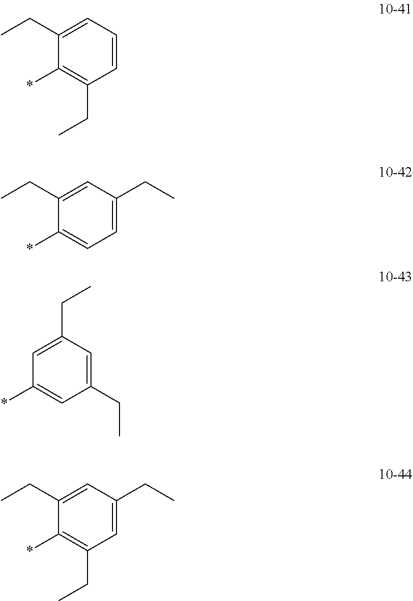

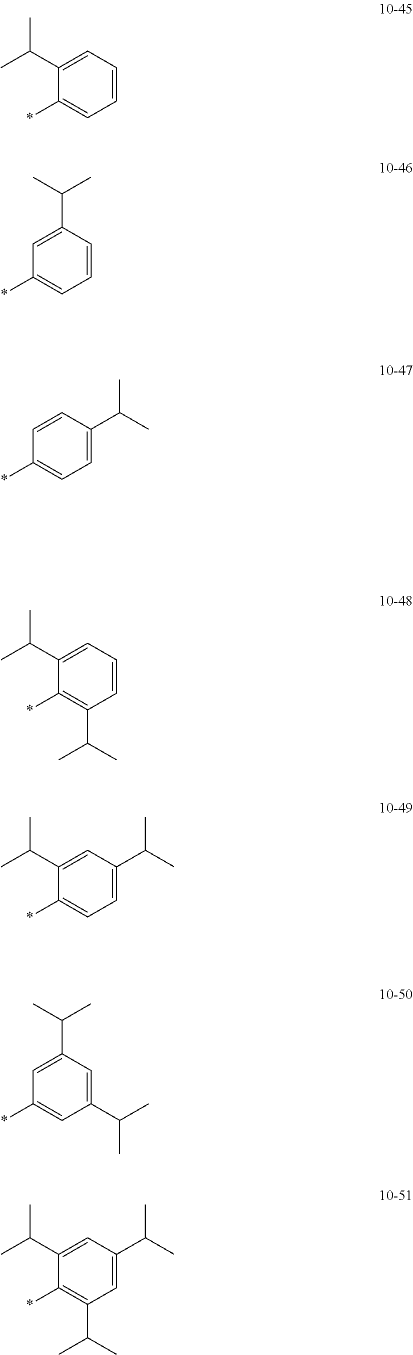

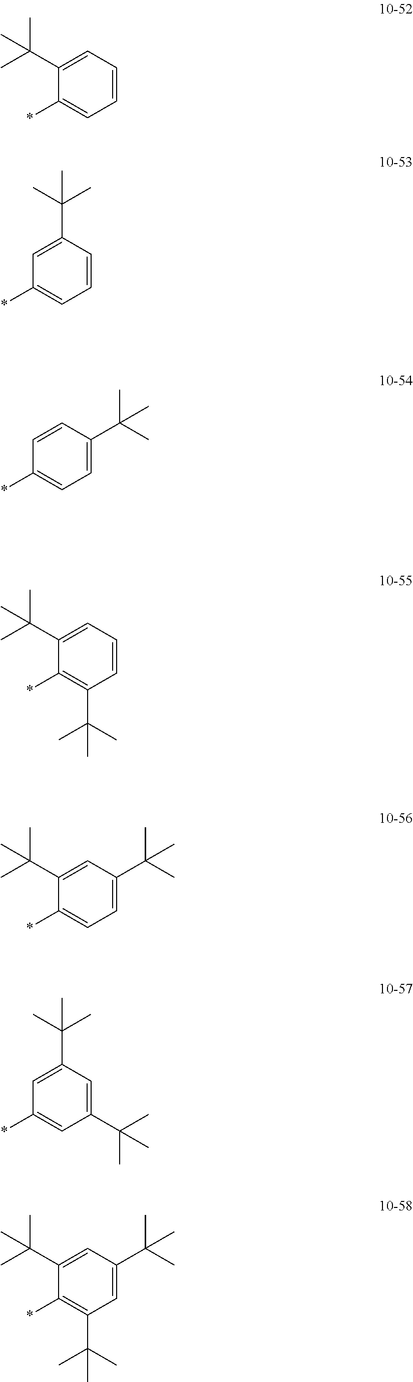

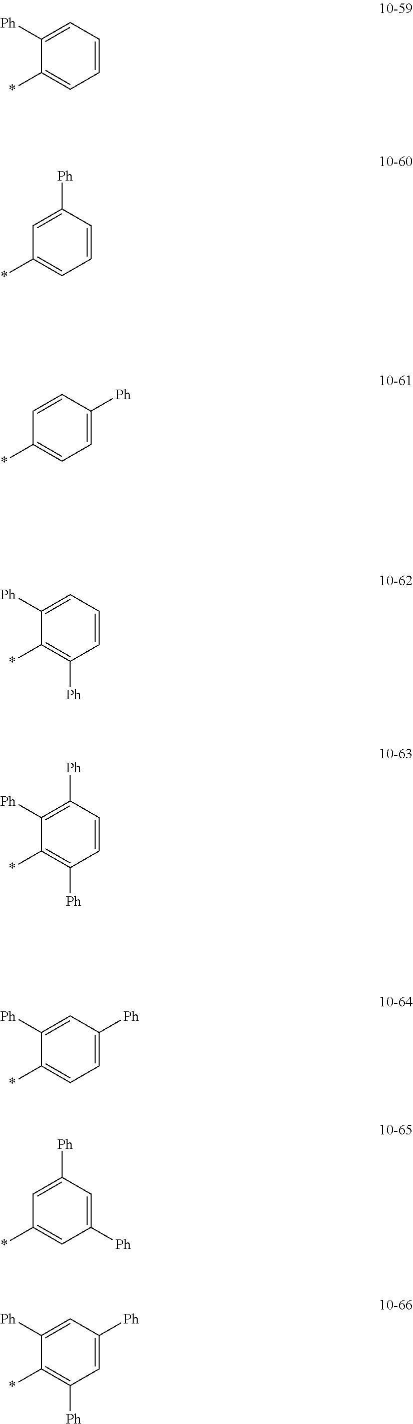

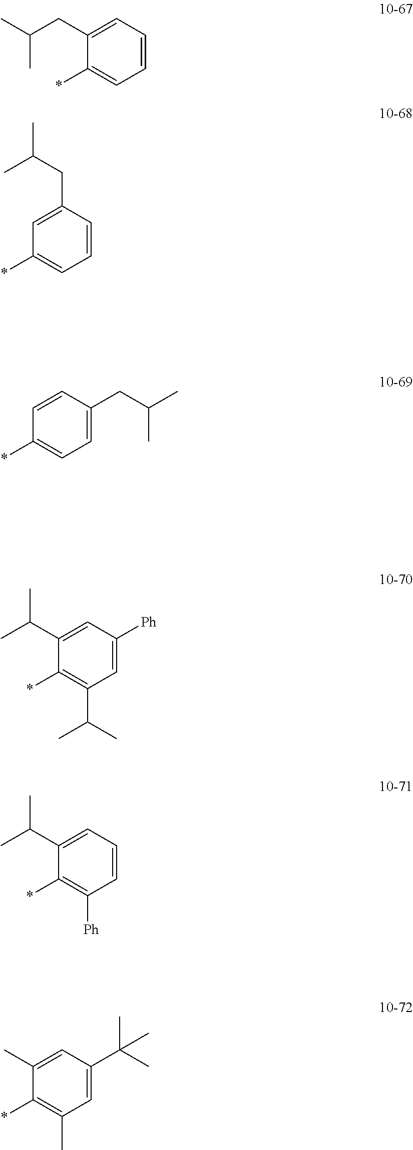

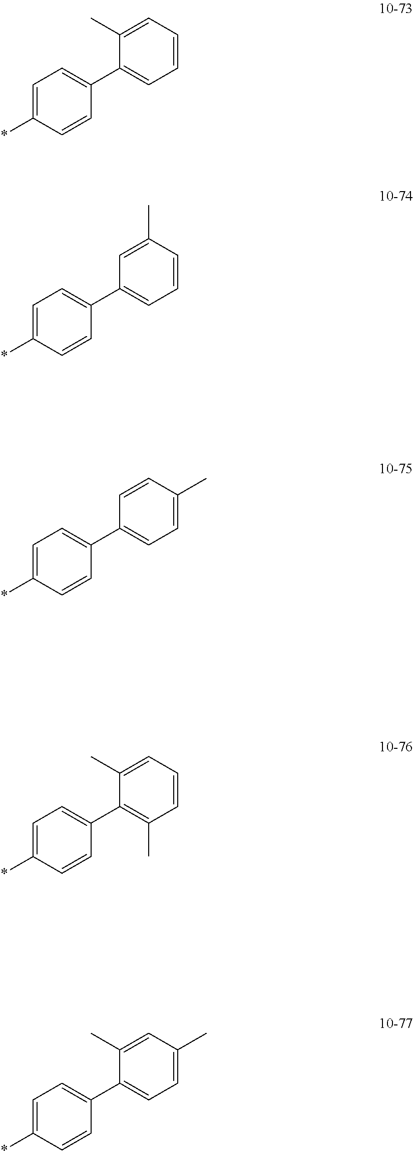 [SMILES: *c1ccc(-c2ccccc2C)cc1 ; *c1ccc(-c2cccc(C)c2)cc1 ; *c1ccc(-c2ccc(C)cc2)cc1 ; *c1ccc(-c2c(C)cccc2C)cc1 ; *c1ccc(-c2ccc(C)cc2C)cc1]

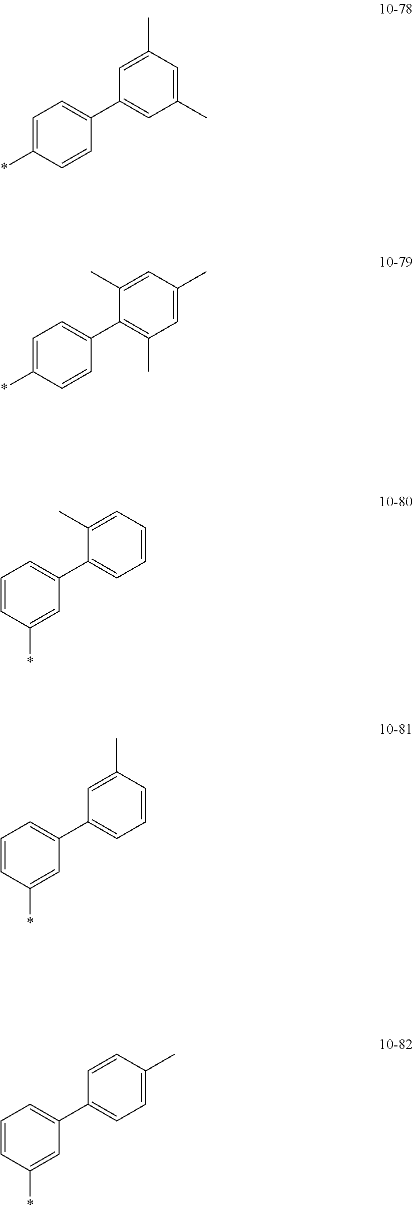 [SMILES: *c1ccc(-c2cc(C)cc(C)c2)cc1 ; *c1ccc(-c2c(C)cc(C)cc2C)cc1 ; *c1cccc(-c2ccccc2C)c1 ; *c1cccc(-c2cccc(C)c2)c1 ; *c1cccc(-c2ccc(C)cc2)c1]

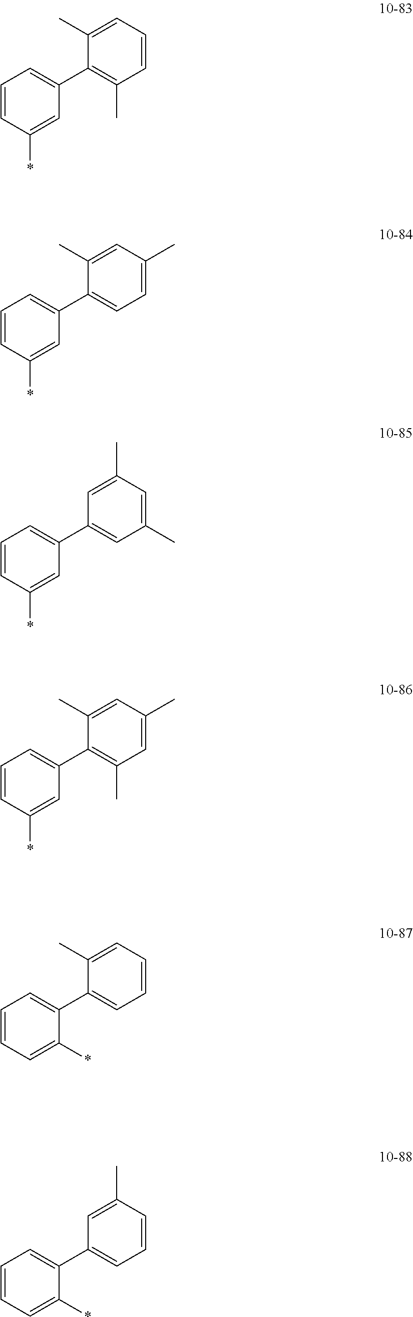 [SMILES: *c1cccc(-c2c(C)cccc2C)c1 ; *c1cccc(-c2ccc(C)cc2C)c1 ; *c1cccc(-c2cc(C)cc(C)c2)c1 ; *c1cccc(-c2c(C)cc(C)cc2C)c1 ; *c1ccccc1-c1ccccc1C ; *c1ccccc1-c1cccc(C)c1]

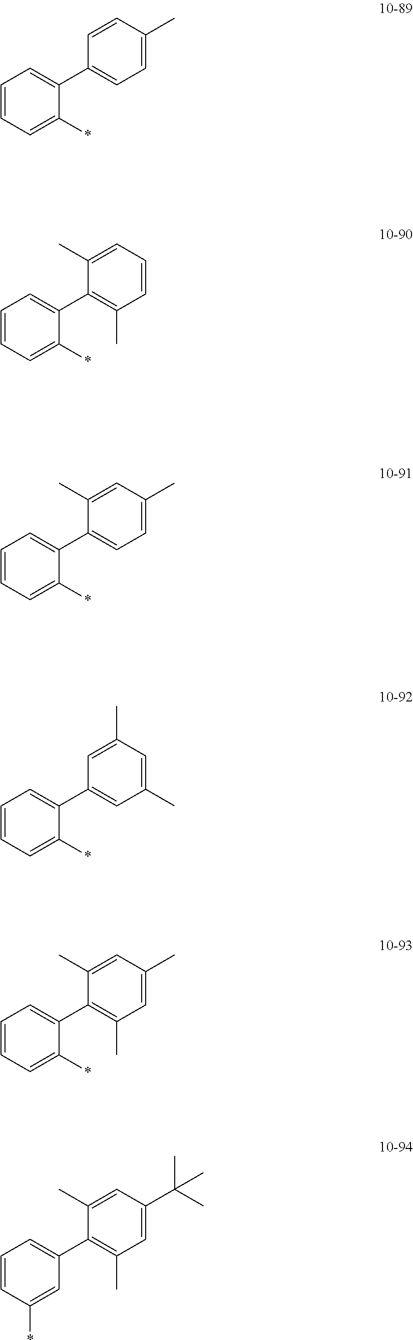 [SMILES: *c1ccccc1-c1ccc(C)cc1 ; *c1ccccc1-c1c(C)cccc1C ; *c1ccccc1-c1ccc(C)cc1C ; *c1ccccc1-c1cc(C)cc(C)c1 ; *c1ccccc1-c1c(C)cc(C)cc1C ; *c1cccc(-c2c(C)cc(C(C)(C)C)cc2C)c1]

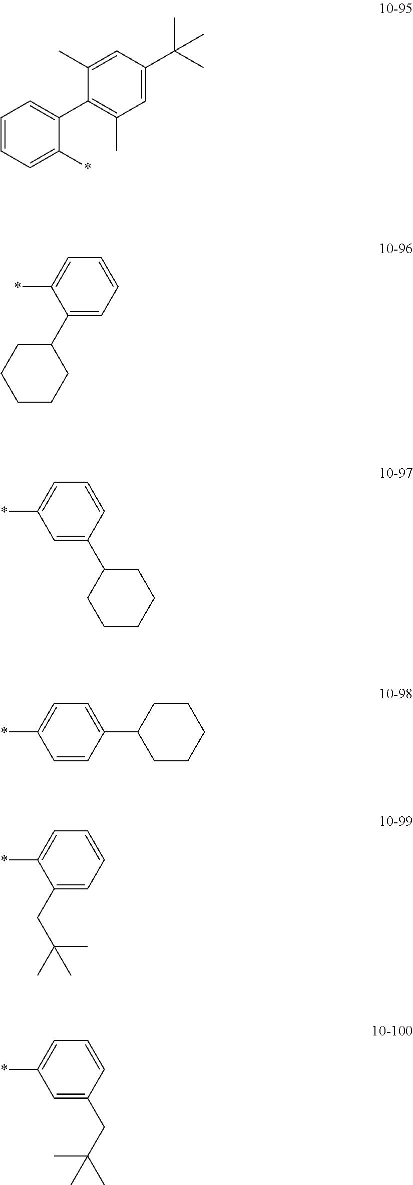 [SMILES: *c1ccccc1-c1c(C)cc(C(C)(C)C)cc1C ; *c1ccccc1C1CCCCC1 ; *c1cccc(C2CCCCC2)c1 ; *c1ccc(C2CCCCC2)cc1 ; *c1ccccc1CC(C)(C)C ; *c1cccc(CC(C)(C)C)c1]

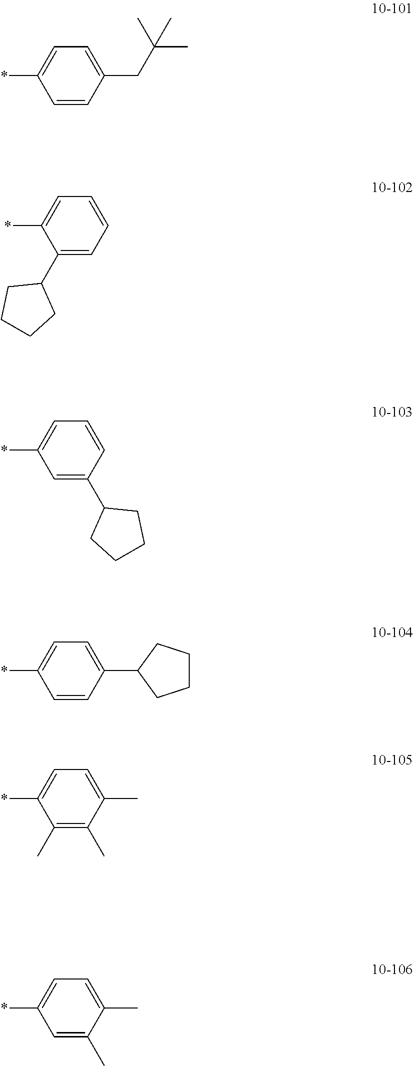

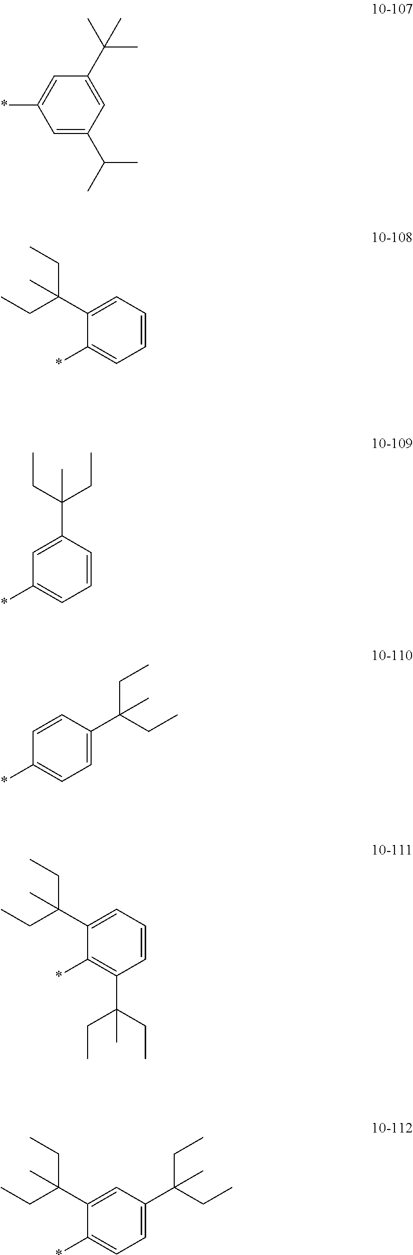

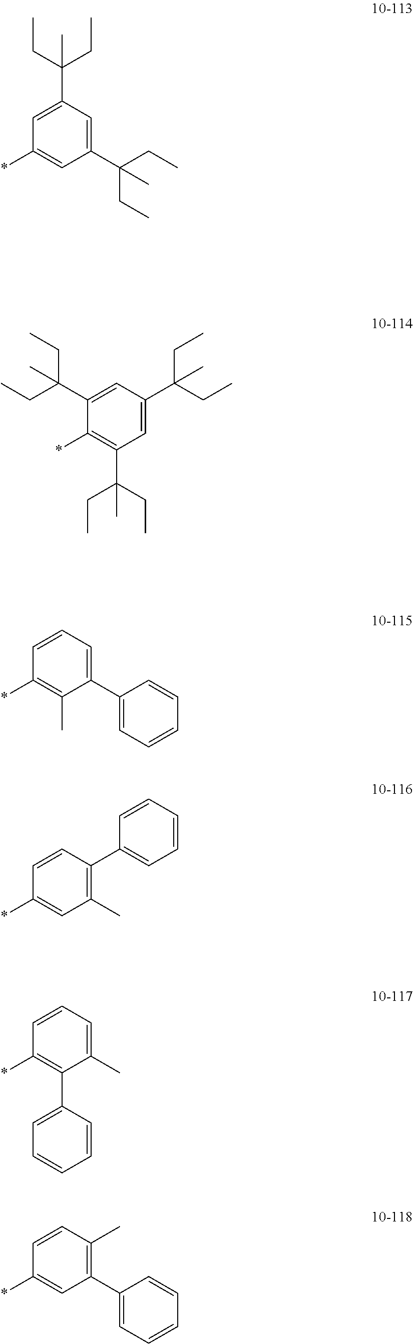

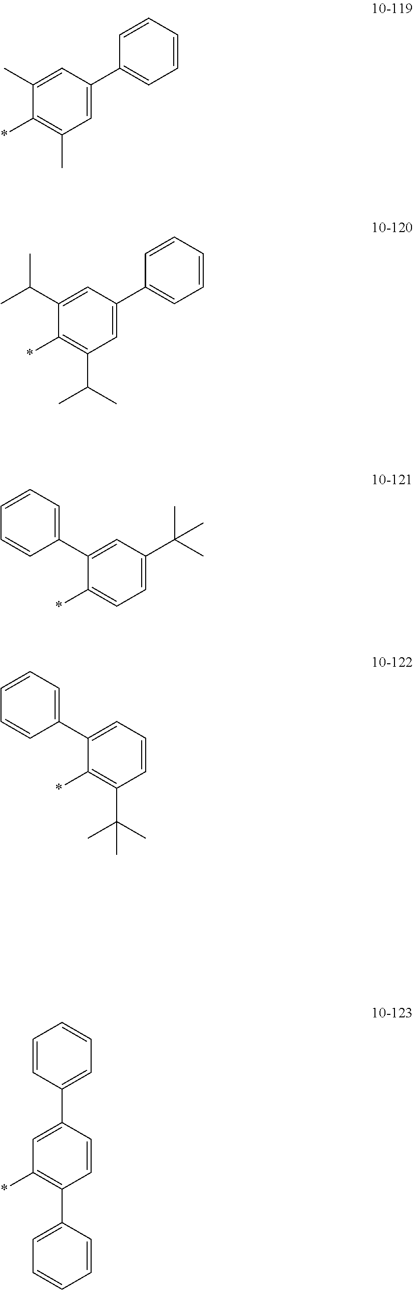 [SMILES: *c1c(C)cc(-c2ccccc2)cc1C ; *c1c(C(C)C)cc(-c2ccccc2)cc1C(C)C ; *c1ccc(C(C)(C)C)cc1-c1ccccc1 ; *c1c(-c2ccccc2)cccc1C(C)(C)C ; *c1cc(-c2ccccc2)ccc1-c1ccccc1]

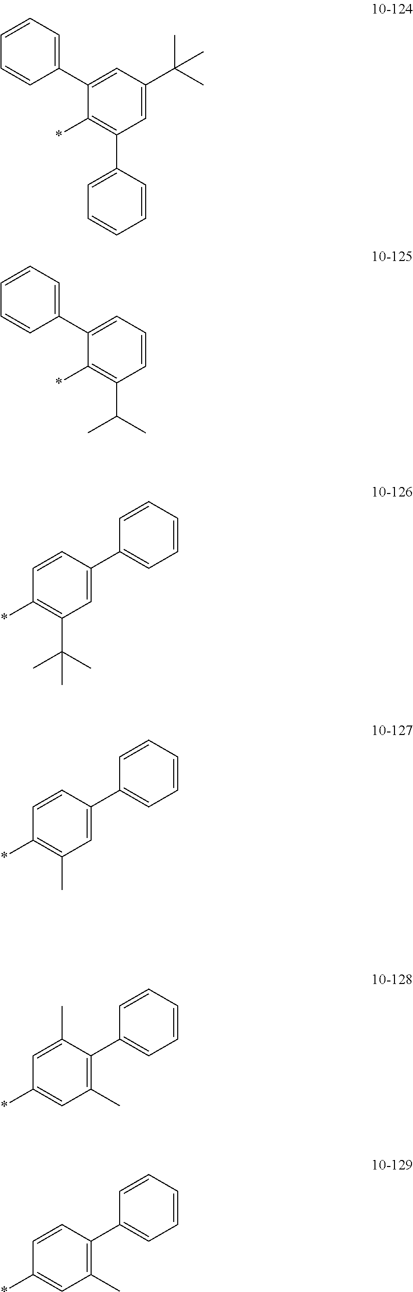 [SMILES: *c1c(-c2ccccc2)cc(C(C)(C)C)cc1-c1ccccc1 ; *c1c(-c2ccccc2)cccc1C(C)C ; *c1ccc(-c2ccccc2)cc1C(C)(C)C ; *c1ccc(-c2ccccc2)cc1C ; *c1cc(C)c(-c2ccccc2)c(C)c1 ; *c1ccc(-c2ccccc2)c(C)c1]

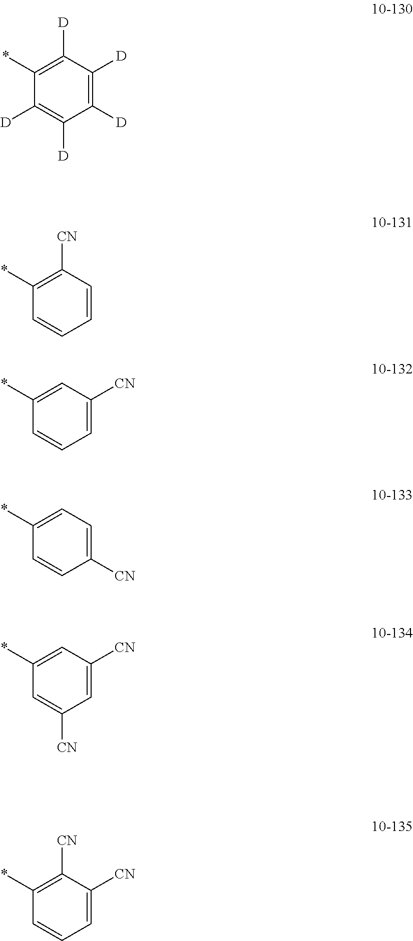 [SMILES: *c1c([2H])c([2H])c([2H])c([2H])c1[2H] ; *c1ccccc1C#N ; *c1cccc(C#N)c1 ; *c1ccc(C#N)cc1 ; *c1cc(C#N)cc(C#N)c1 ; *c1cccc(C#N)c1C#N]

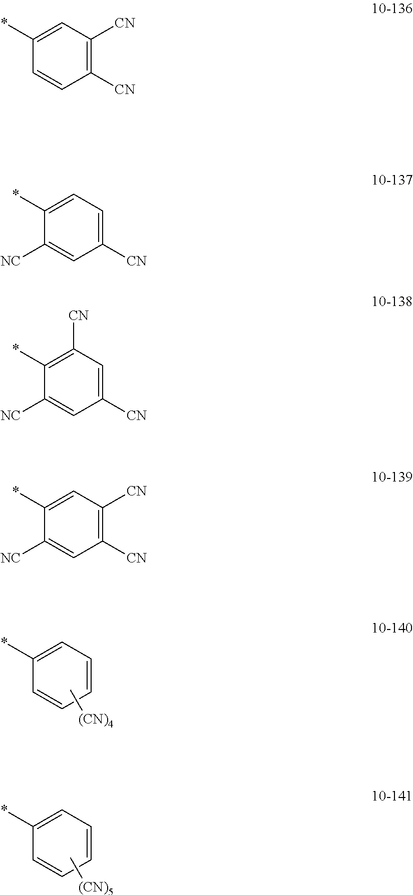

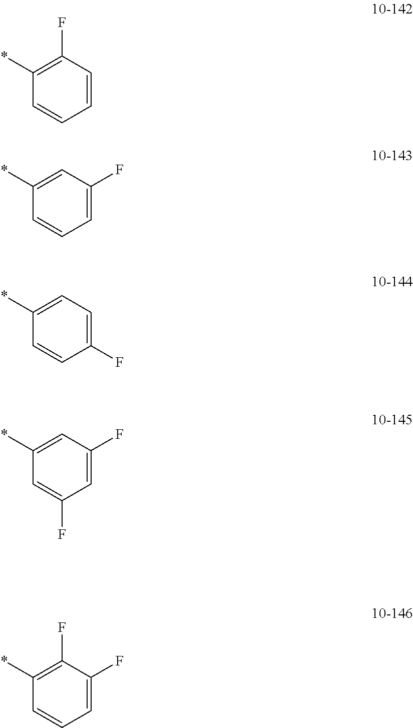 [SMILES: *c1ccccc1F ; *c1cccc(F)c1 ; *c1ccc(F)cc1 ; *c1cc(F)cc(F)c1 ; *c1cccc(F)c1F]

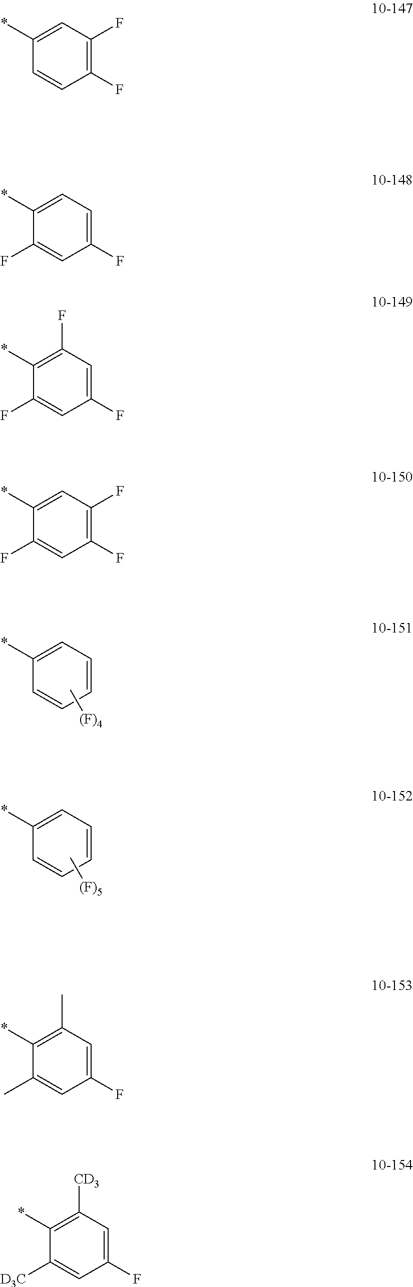

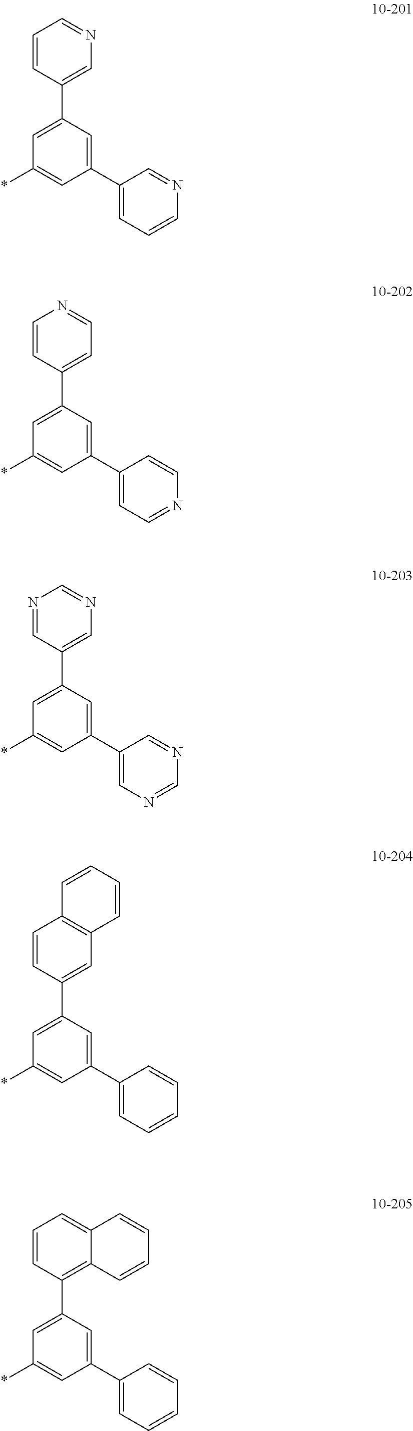 [SMILES: *c1cc(-c2cccnc2)cc(-c2cccnc2)c1 ; *c1cc(-c2ccncc2)cc(-c2ccncc2)c1 ; *c1cc(-c2cncnc2)cc(-c2cncnc2)c1 ; *c1cc(-c2ccccc2)cc(-c2ccc3ccccc3c2)c1 ; *c1cc(-c2ccccc2)cc(-c2cccc3ccccc23)c1]

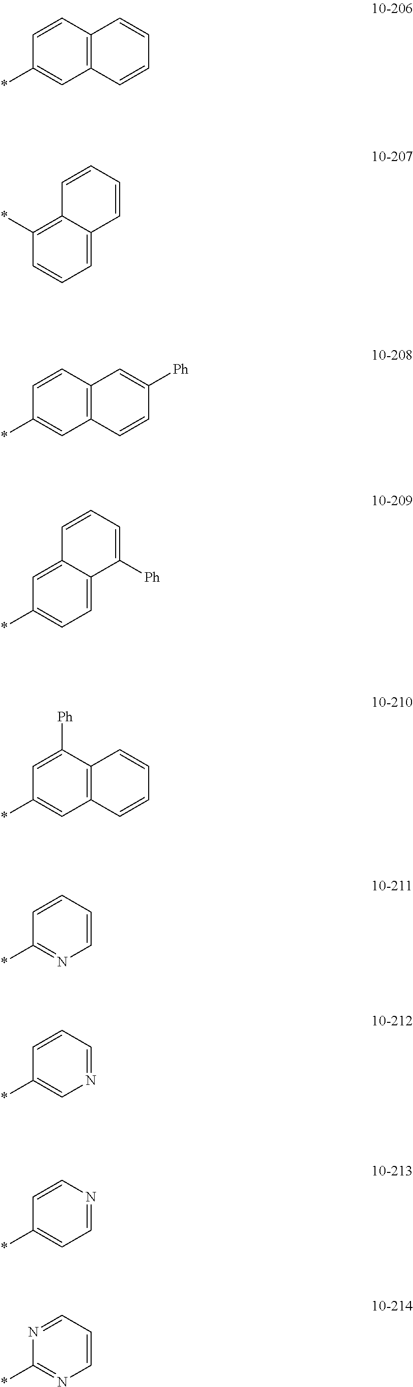

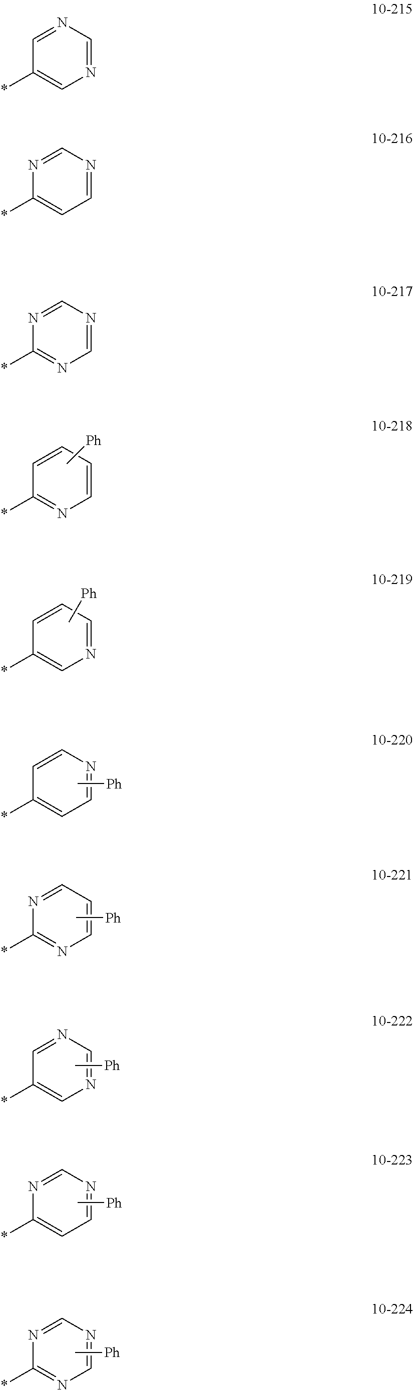

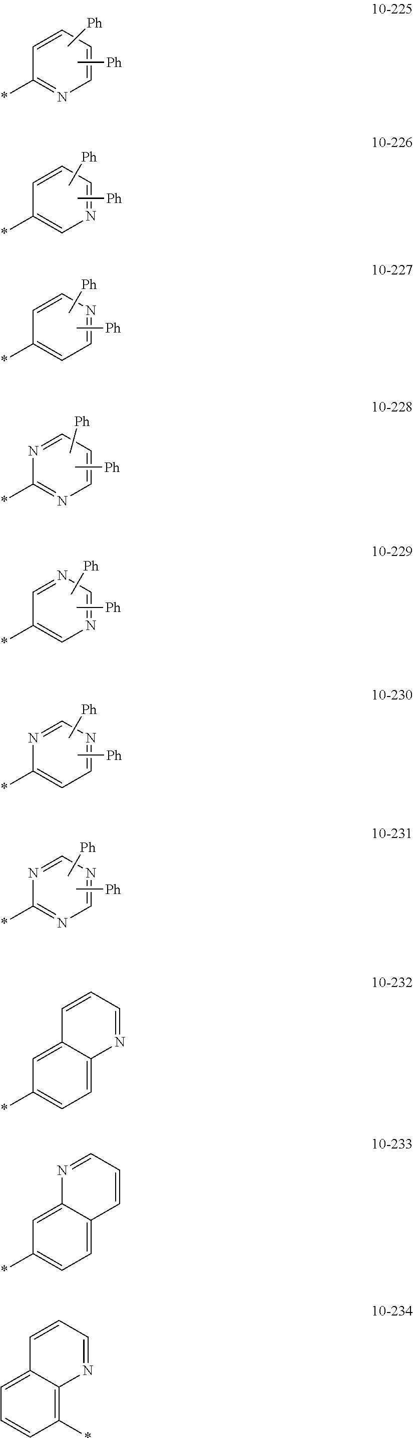

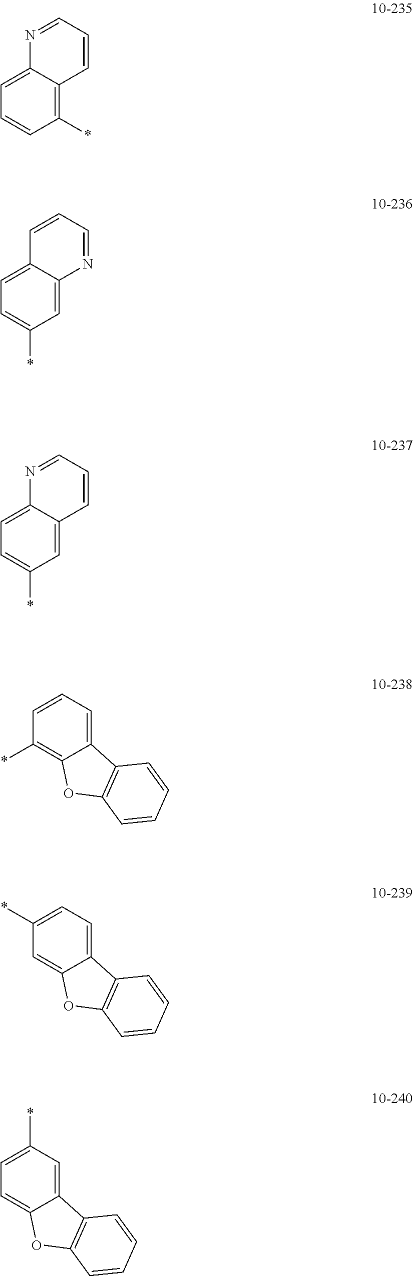 [SMILES: *c1cccc2ncccc12 ; *c1ccc2cccnc2c1 ; *c1ccc2ncccc2c1 ; *c1cccc2c1oc1ccccc12 ; *c1ccc2c(c1)oc1ccccc12 ; *c1ccc2oc3ccccc3c2c1]

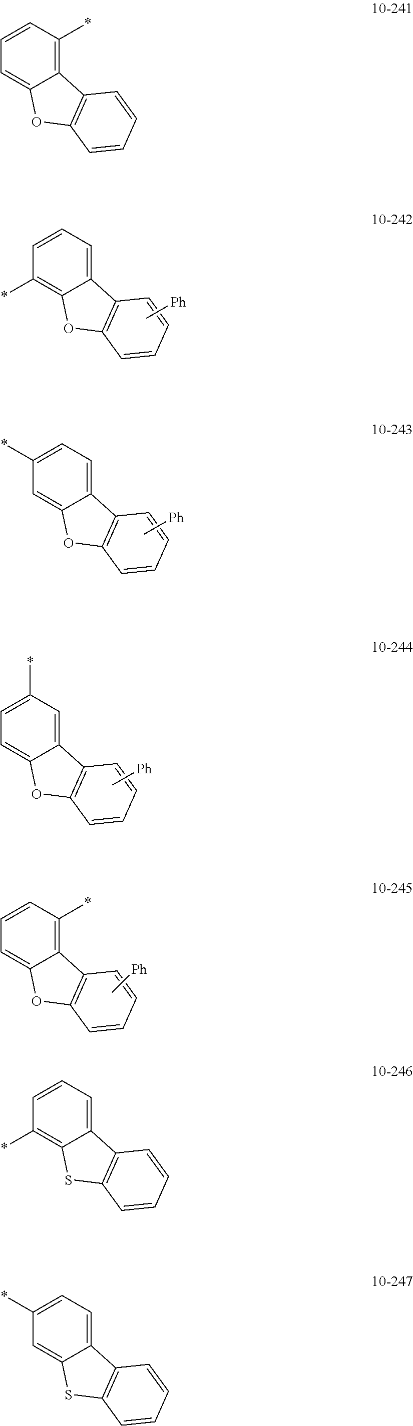

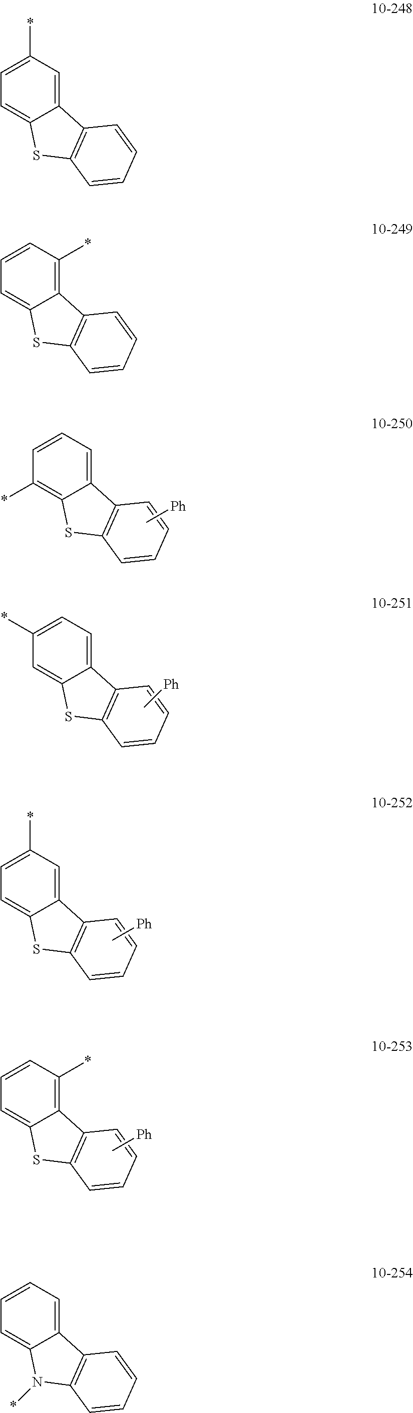

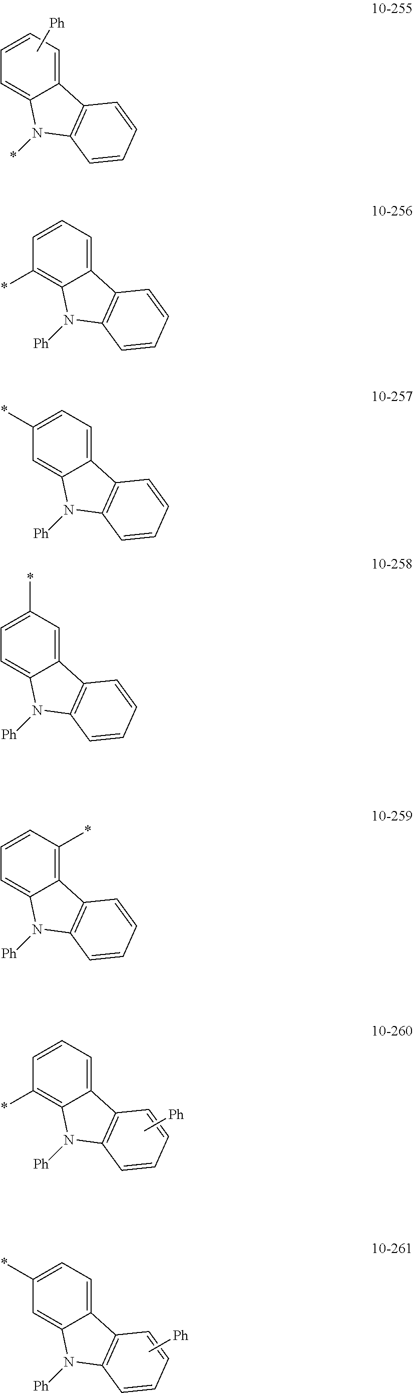

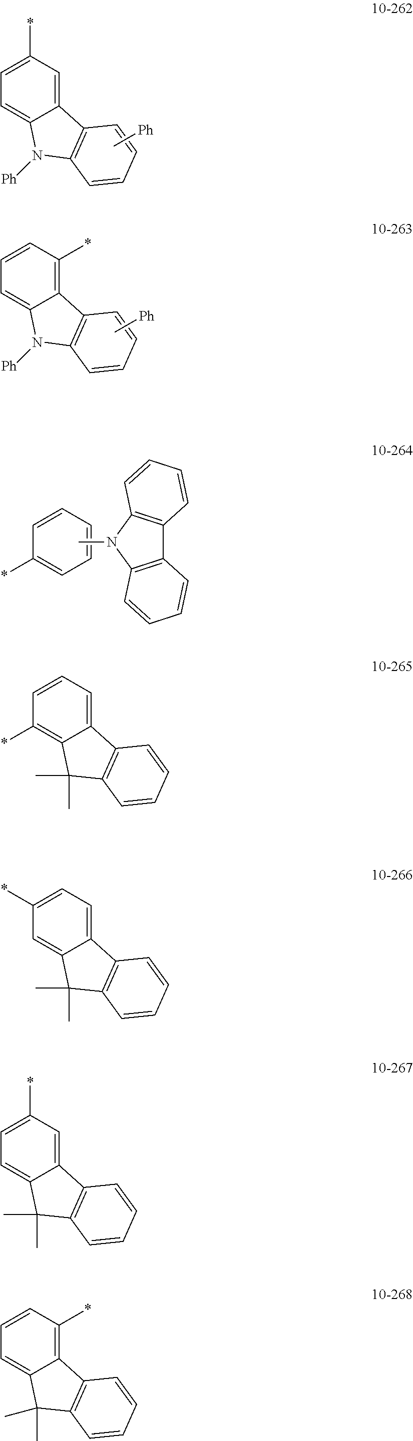

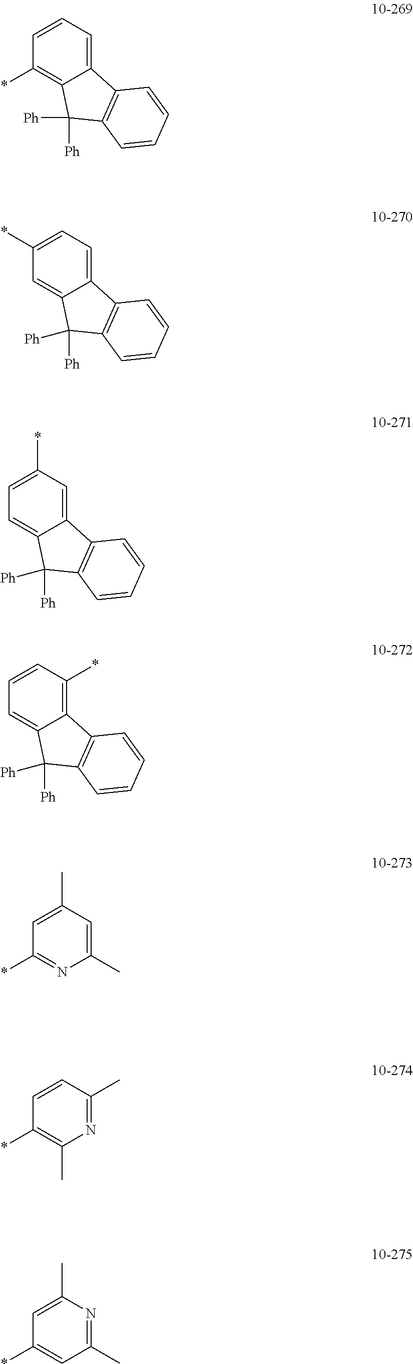

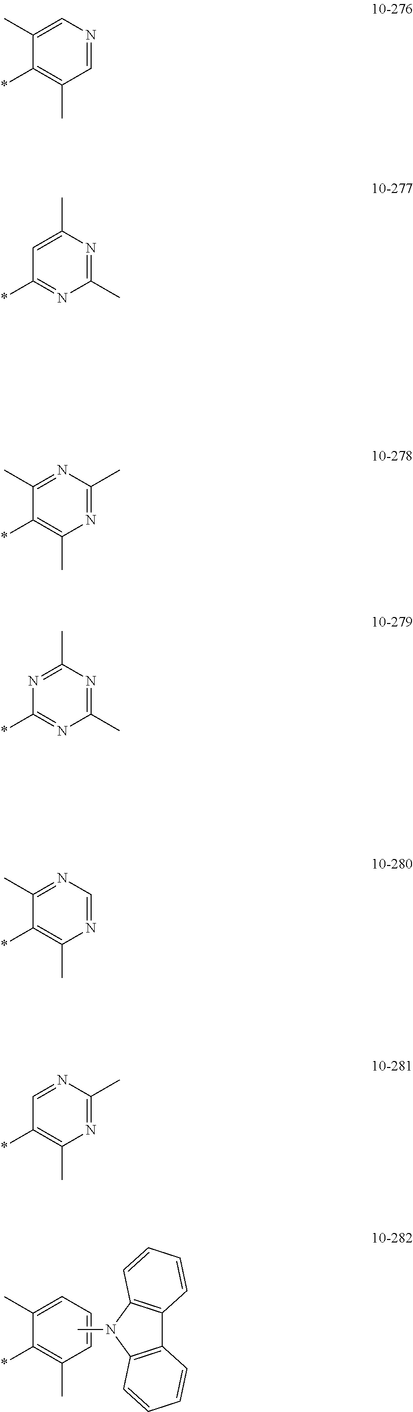

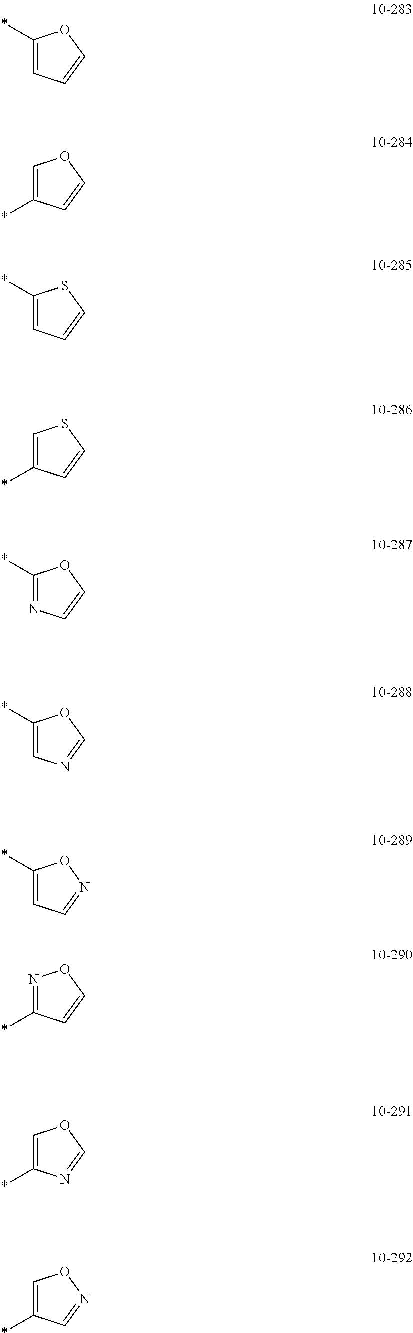

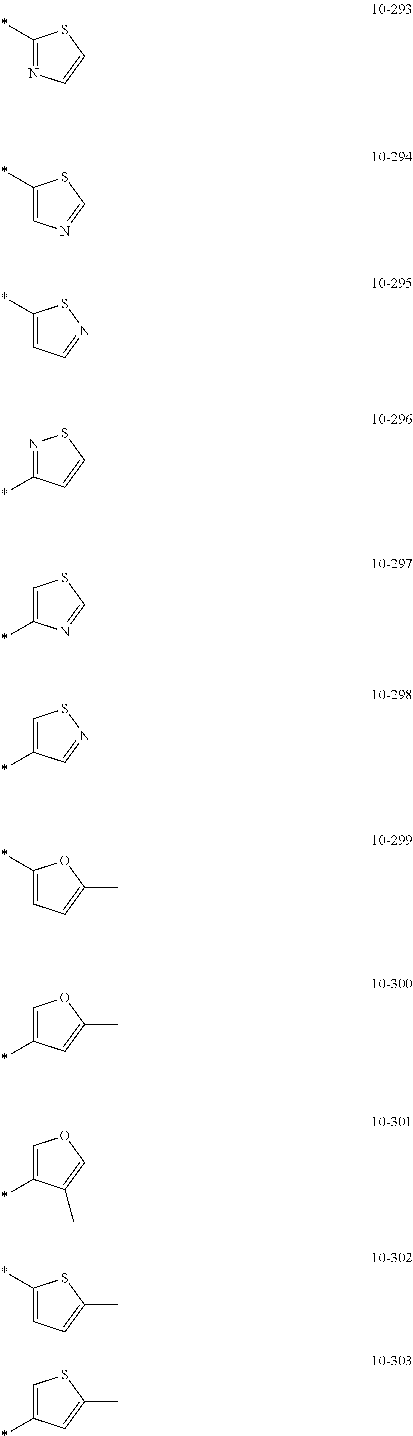

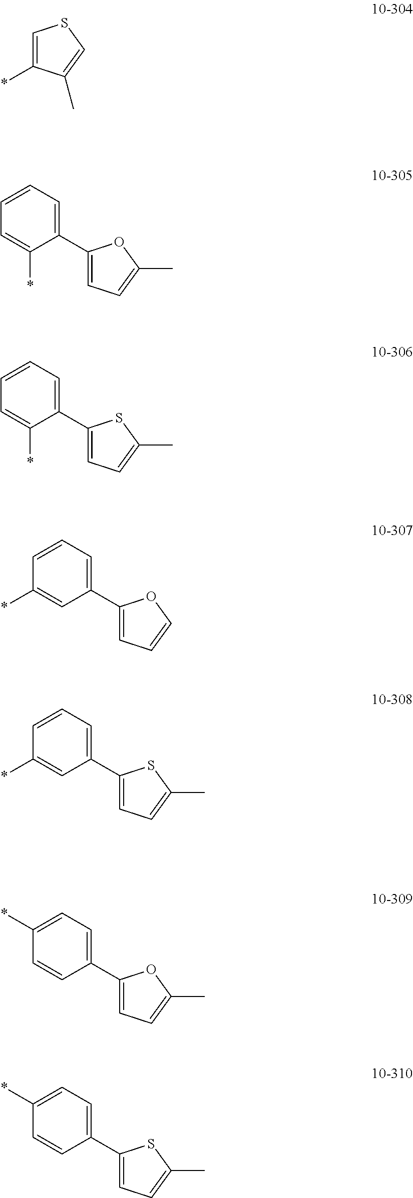

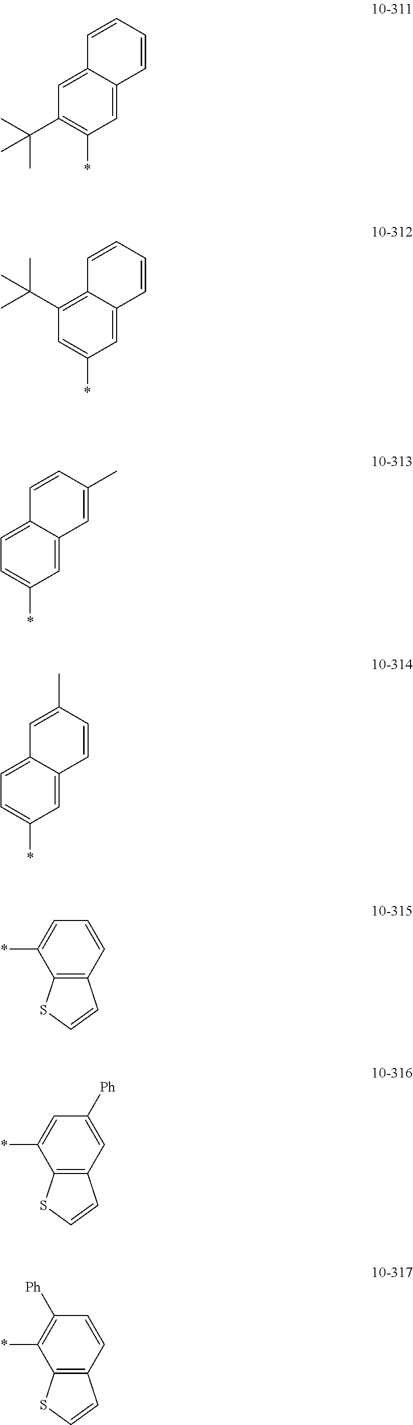

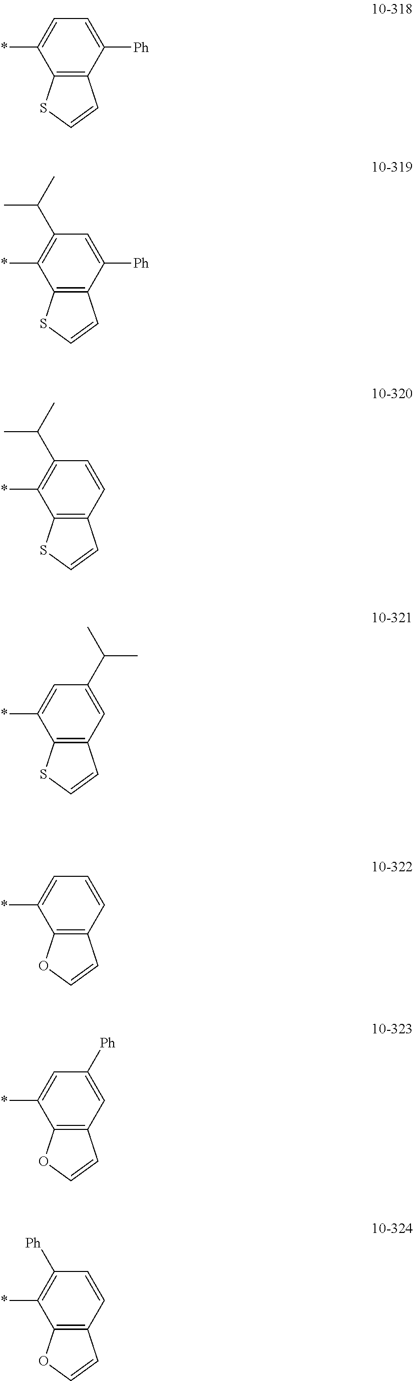

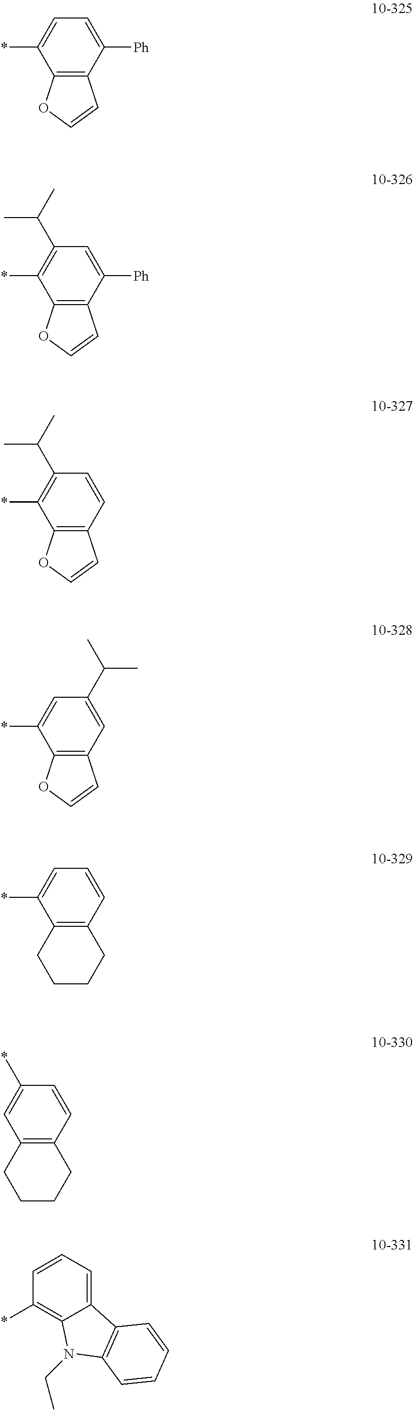

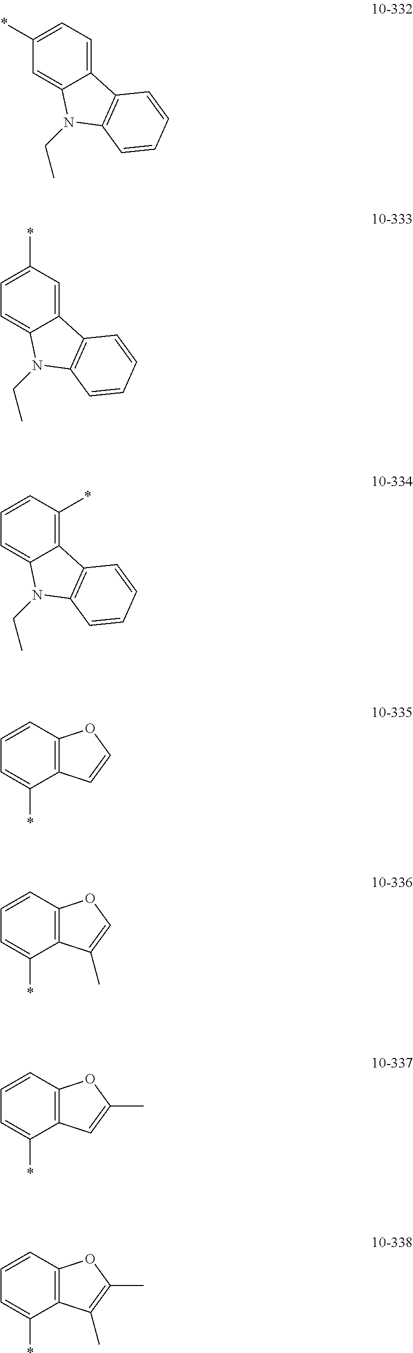

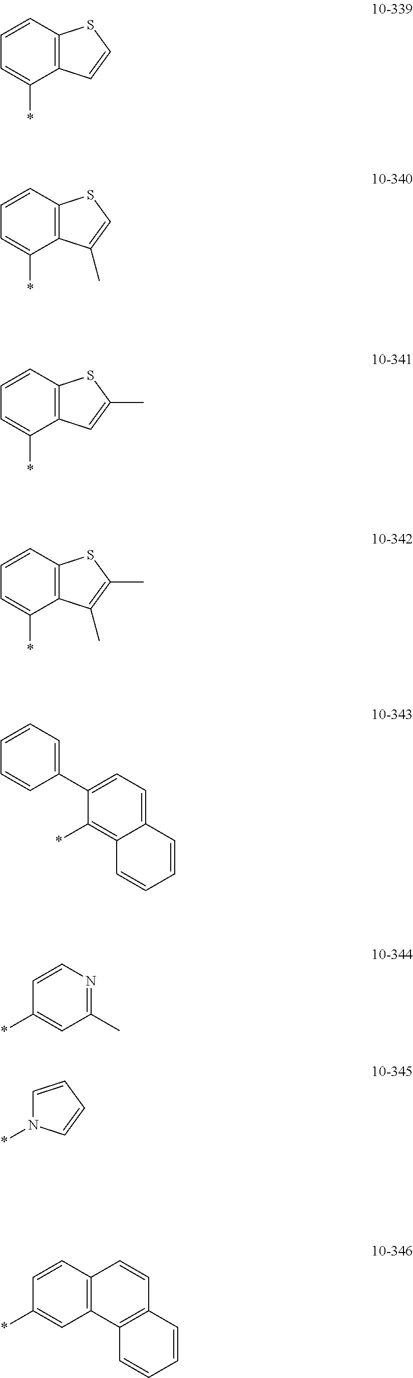

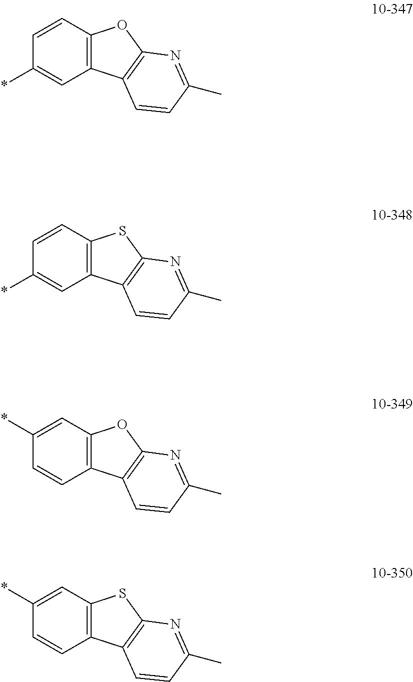 [SMILES: *c1ccc2oc3nc(C)ccc3c2c1 ; *c1ccc2sc3nc(C)ccc3c2c1 ; *c1ccc2c(c1)oc1nc(C)ccc12 ; *c1ccc2c(c1)sc1nc(C)ccc12]

* in Formulae 9-1 to 9-67, 9-201 to 9-244, 10-1 to 10-154, and 10-201 to 10-350 indicates a binding site to a neighboring atom, Ph is a phenyl group TMS is a trimethylsilyl group, and TMG is a trimethylgermyl group.

In one or more embodiments, the moiety *-(L₁)ₐ₁-(Ar₁)ₖ₁ in Formula 1B may be a group represented by one of Formulae 3-1 to 3-18:

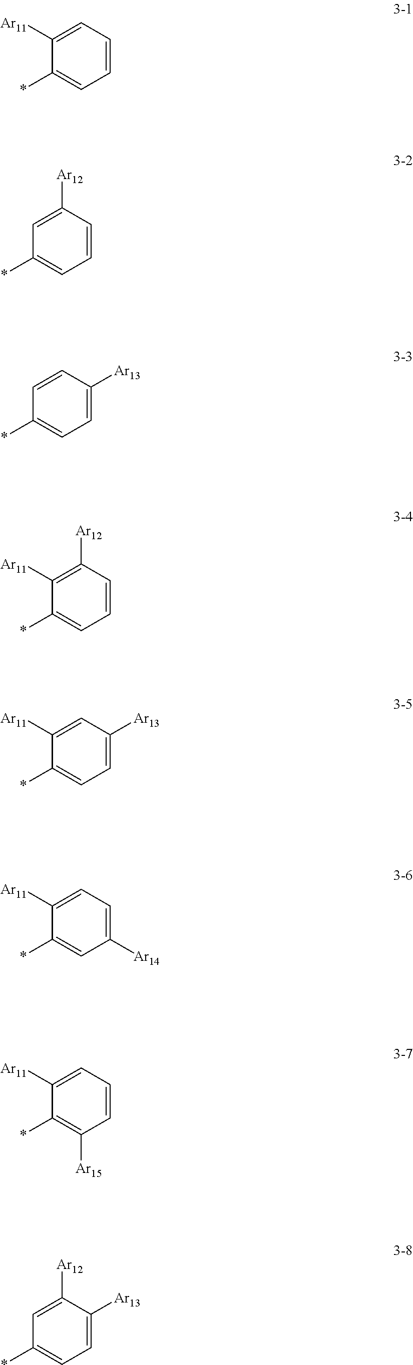

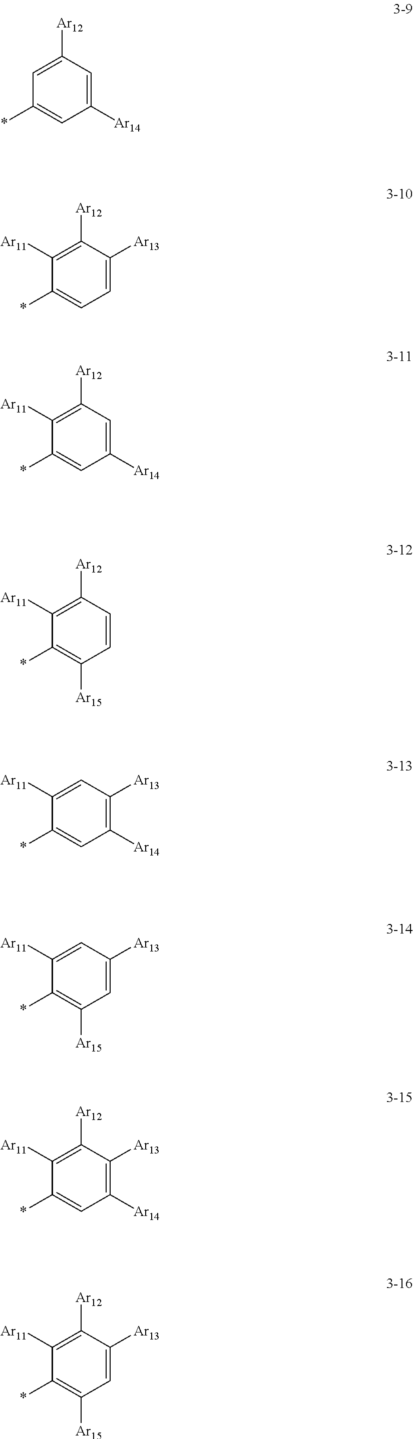

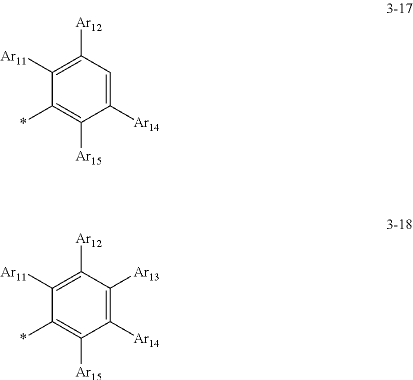

In Formulae 3-1 to 3-18,

Ar₁₁ to Ar₁₅ are each independently the same as described in connection with Ar₁,

* indicates a binding site to a neighboring atom.

In one or more embodiments, Q₁ to Q₉, Q₁₁ to Q₁₉, Q₂₁ to Q₂₉ and Q₃₁ to Q₃₉ described herein may each independently be:

—CH₃, —CD₃, —CD₂H, —CDH₂, —CH₂CH₃, —CH₂CD₃, —CH₂CD₂H, —CH₂CDH₂, —CHDCH₃, —CHDCD₂H, —OHDCDH₂, —CHDCD₃, —CD₂CD₃, —CD₂CD₂H, or —CD₂CDH₂;

an n-propyl group, an isopropyl group, an n-butyl group, an isobutyl group, a sec-butyl group, a tert-butyl group, an n-pentyl group, an isopentyl group, a sec-pentyl group, a tert-pentyl group, a phenyl group, or a naphthyl group; or

an n-propyl group, an isopropyl group, an n-butyl group, an isobutyl group, a sec-butyl group, a tert-butyl group, an n-pentyl group, an isopentyl group, a sec-pentyl group, a tert-pentyl group, a phenyl group, or a naphthyl group, each substituted with deuterium, a C₁-C₁₀ alkyl group, a phenyl group, or a combination thereof.

In one or more embodiments, at least one of Ar₁, R₁₀, R₂₀, R₃₀, and R₄₀ may include —F.

For example, at least one of Ar₁, R₁₀, R₂₀, R₃₀, and R₄₀ may be —F or a substituent substituted with —F.

In one or more embodiments, one to four of Ar₁, R₁₀, R₂₀, R₃₀, and R₄₀ may include —F.

For example, one to four of Ar₁, R₁₀, R₂₀, R₃₀, and R₄₀ may be —F or a substituent substituted with —F.

In one or more embodiments, one or two of Ar₁, R₁₀, R₂₀, R₃₀, and R₄₀ may include —F.

For example, at least one of Ar₁, R₁₀, R₂₀, R₃₀, and R₄₀ may be —F.

In one or more embodiments, at least one of R₁₀, R₂₀, R₃₀, and R₄₀ may include —F.

For example, one to four of Ar₁, R₁₀, R₂₀, R₃₀, and R₄₀ may be —F or a substituent group substituted with —F.

In one or more embodiments, at least one of R₂₀ and R₄₀ may include —F.

For example, at least one of R₂₀ and R₄₀ may be —F or a substituent group substituted with —F.

In one or more embodiments, at least one of R₁₀, R₂₀, R₃₀, and R₄₀ may be —F or a substituent group substituted with —F, Ar₁ may be —F or a substituent group substituted with —F.

The term “substituent group substituted with —F” as used herein refers to a group that is substituted with at least on —F atom. The substituent group may be, example, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₁-C₆₀ alkylthio group, a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₂-C₁₀ heterocycloalkenyl group, a substituted or unsubstituted C₆-C₆₀ aryl group, a substituted or unsubstituted C₇-C₆₀ alkyl aryl group, a substituted or unsubstituted C₇-C₆₀ aryl alkyl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted C₂-C₆₀ alkyl heteroaryl group, a substituted or unsubstituted C₂-C₆₀ heteroaryl alkyl group, a substituted or unsubstituted C₁-C₆₀ heteroaryloxy group, a substituted or unsubstituted C₁-C₆₀ heteroarylthio group, a substituted or unsubstituted monovalent non-aromatic condensed polycyclic group, a substituted or unsubstituted monovalent non-aromatic condensed heteropolycyclic group, —N(Q₁)(Q₂), —Si(Q₃)(Q₄)(Q₅), —Ge(Q₃)(Q₄)(Q₅), —B(Q₆)(Q₇), —C(═O)(Q₆), —S(═O)₂(Q₆), —P(═O)(Q₈)(Q₉), or —P(Q₈)(Q₉). It is to be understood that the “unsubstituted” groups above are each substituted with at least one —F, whereas the “substituted” groups above are each substituted with at least one —F and are further substituted with one or more different groups.

In one or more embodiments, the moiety

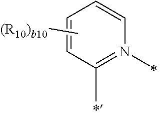

in Formula 1A may be represented by one of Formulae 1-1 to 1-16:

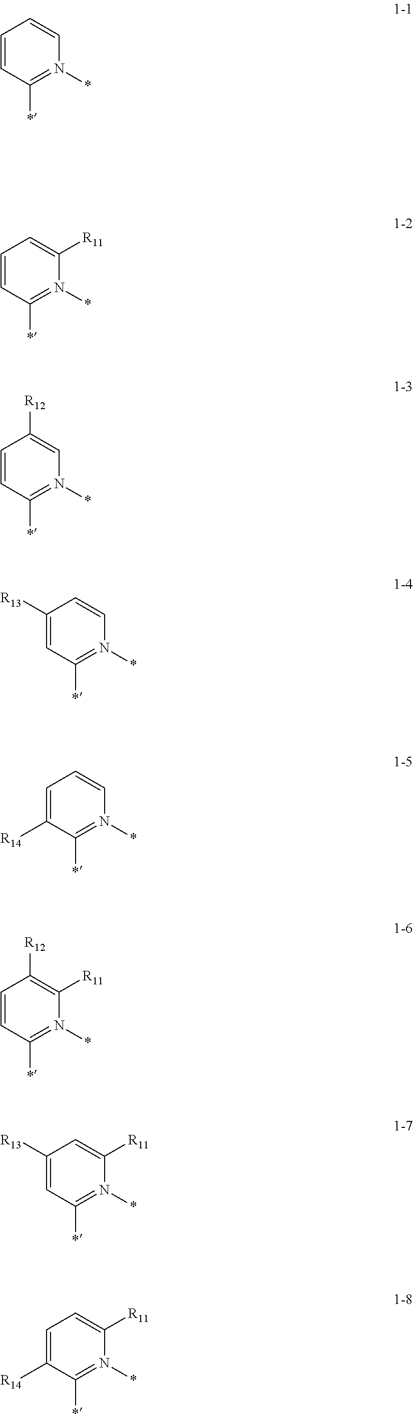

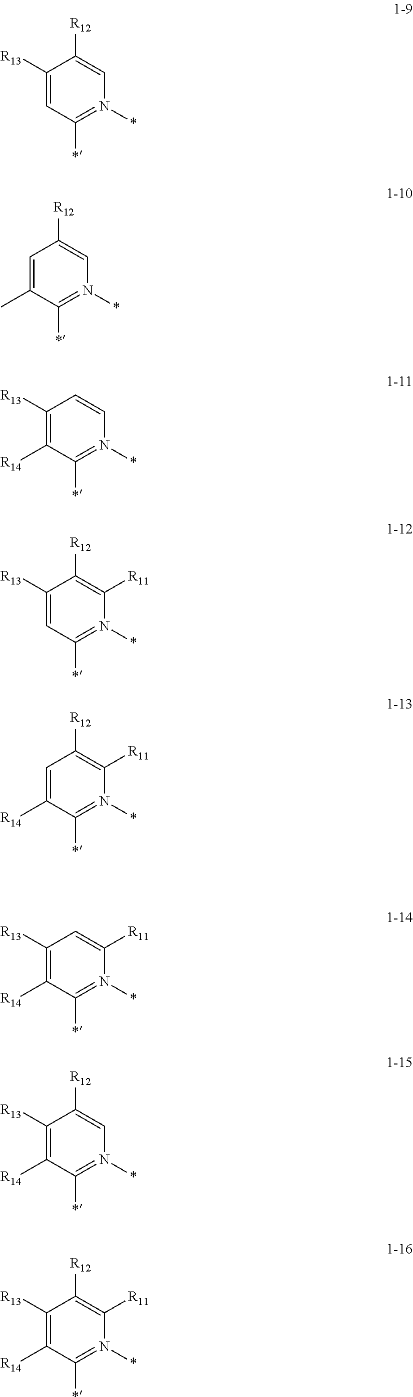

wherein, in Formulae 1-1 to 1-16,

R₁₁ to R₁₄ may each independently be the same as described in connection with R₁₀.

In one or more embodiments, each of R₁₁ to R₁₄ may not be hydrogen.

For example, R₁₁ to R₁₄ may each independently be deuterium, —F, —Cl, —Br, —I, —CD₃, —CD₂H, —CDH₂, —CF₃, —CF₂H, —CFH₂, a methyl group, an ethyl group, an n-propyl group, an isopropyl group, an n-butyl group, an isobutyl group, a sec-butyl group, a tert-butyl group, an n-pentyl group, an isopentyl group, a 2-methylbutyl group, a sec-pentyl group, a tert-pentyl group, a neo-pentyl group, 3-pentyl group, 3-methyl-2-butyl group, a phenyl group, a biphenyl group, a naphthyl group, —Si(Q₁)(Q₂)(Q₃), or —Ge(Q₁)(Q₂)(Q₃),

Q₁ to Q₃ may each independently be:

—CH₃, —CD₃, —CD₂H, —CDH₂, —CH₂CH₃, —CH₂CD₃, —CH₂CD₂H, —CH₂CDH₂, —CHDCH₃, —CHDCD₂H, —CHDCDH₂, —CHDCD₃, —CD₂CD₃, —CD₂CD₂H, or —CD₂CDH₂;

an n-propyl group, an isopropyl group, an n-butyl group, an isobutyl group, a sec-butyl group, a tert-butyl group, an n-pentyl group, an isopentyl group, a sec-pentyl group, a tert-pentyl group, a phenyl group, or a naphthyl group; or

an n-propyl group, an isopropyl group, an n-butyl group, an isobutyl group, a sec-butyl group, a tert-butyl group, an n-pentyl group, an isopentyl group, a sec-pentyl group, a tert-pentyl group, a phenyl group, or a naphthyl group, each substituted with deuterium, a C₁-C₁₀ alkyl group, a phenyl group, or a combination thereof.

* and *′ in Formulae 1-1 to 1-16 each indicate a binding site to a neighboring atom.

In one or more embodiments, the moiety

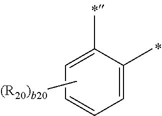

in Formula 1A may be represented by one of Formulae 2-1 to 2-16:

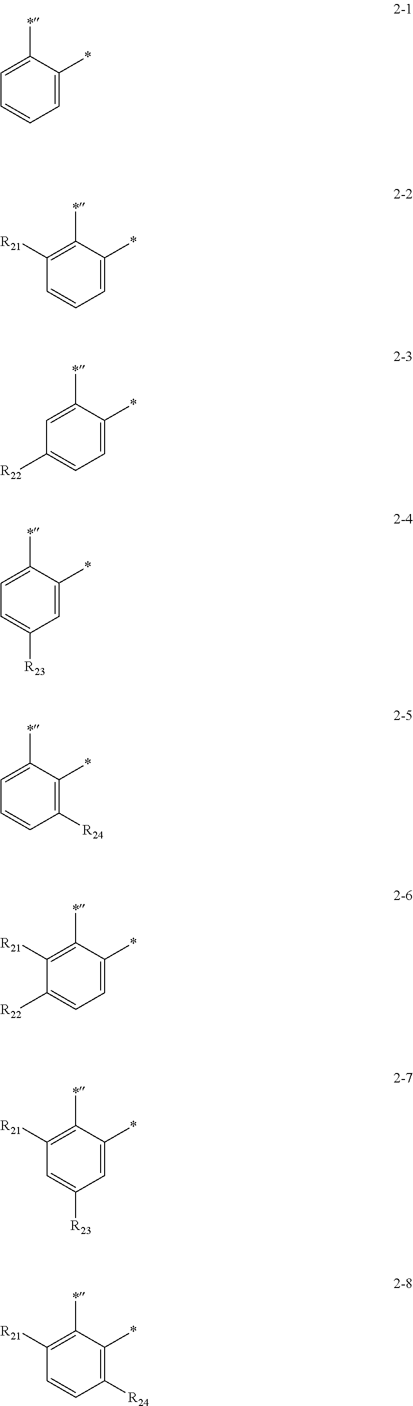

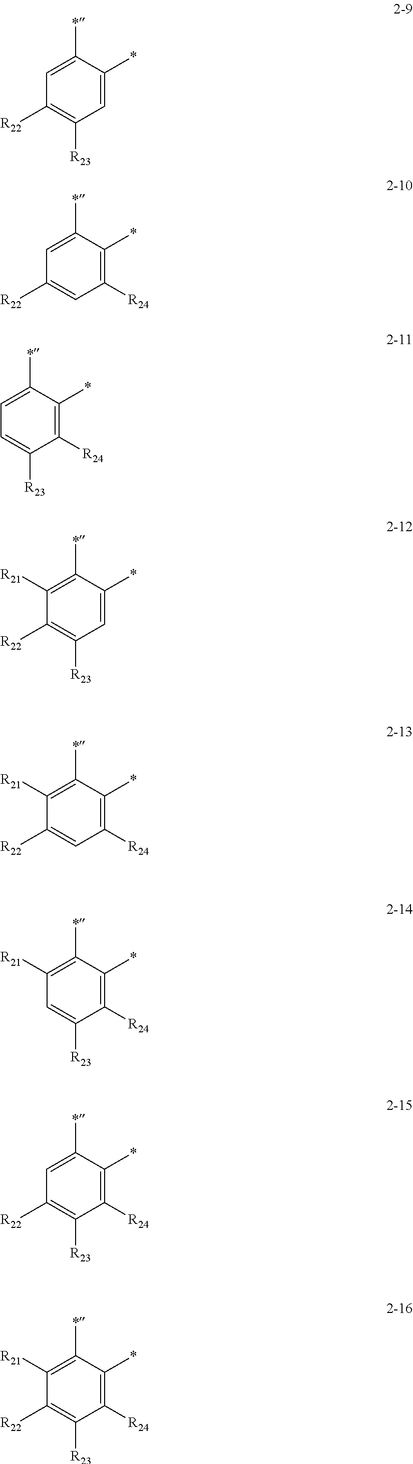

wherein, in Formulae 2-1 to 2-16,

R₂₁ to R₂₄ may each independently be the same as described in connection with R₂₀.

In one or more embodiments, each of R₂₁ to R₂₄ may not be hydrogen.

For example, R₂₁ to R₂₄ may each independently be deuterium, —F, —Cl, —Br, —I, —CD₃, —CD₂H, —CDH₂, —CF₃, —CF₂H, —CFH₂, a methyl group, an ethyl group, an n-propyl group, an isopropyl group, an n-butyl group, an isobutyl group, a sec-butyl group, a tert-butyl group, an n-pentyl group, an isopentyl group, a 2-methylbutyl group, a sec-pentyl group, a tert-pentyl group, a neo-pentyl group, 3-pentyl group, 3-methyl-2-butyl group, a phenyl group, a biphenyl group, a naphthyl group, —Si(Q₁)(Q₂)(Q₃), or —Ge(Q₁)(Q₂)(Q₃),

Q₁ to Q₃ may each independently be:

—CH₃, —CD₃, —CD₂H, —CDH₂, —CH₂CH₃, —CH₂CD₃, —CH₂CD₂H, —CH₂CDH₂, —CHDCH₃, —CHDCD₂H, —CHDCDH₂, —CHDCD₃, —CD₂CD₃, —CD₂CD₂H, or —CD₂CDH₂; an n-propyl group, an isopropyl group, an n-butyl group, an isobutyl group, a sec-butyl group, a tert-butyl group, an n-pentyl group, an isopentyl group, a sec-pentyl group, a tert-pentyl group, a phenyl group, or a naphthyl group; or an n-propyl group, an isopropyl group, an n-butyl group, an isobutyl group, a sec-butyl group, a tert-butyl group, an n-pentyl group, an isopentyl group, a sec-pentyl group, a tert-pentyl group, a phenyl group, or a naphthyl group, each substituted with deuterium, a C₁-C₁₀ alkyl group, a phenyl group, or a combination thereof.

* and *″ in Formulae 2-1 to 2-16 each indicate a binding site to a neighboring atom.

In one or more embodiments, the organometallic compound may be a group represented by one of Formulae 11-1 to 11-24:

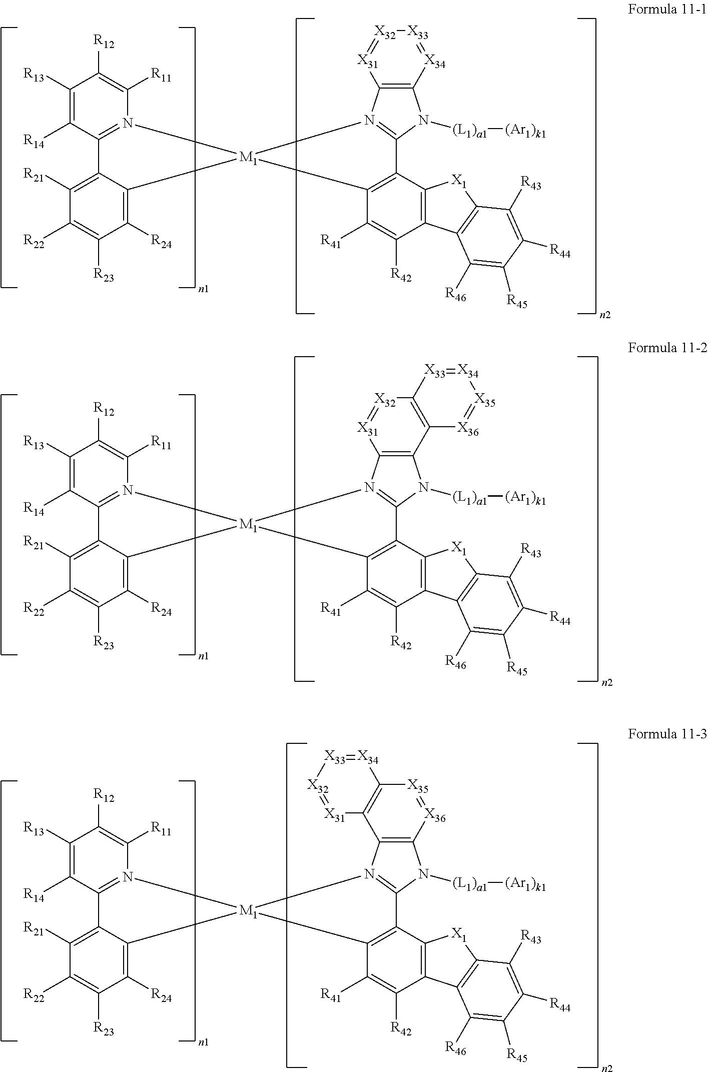

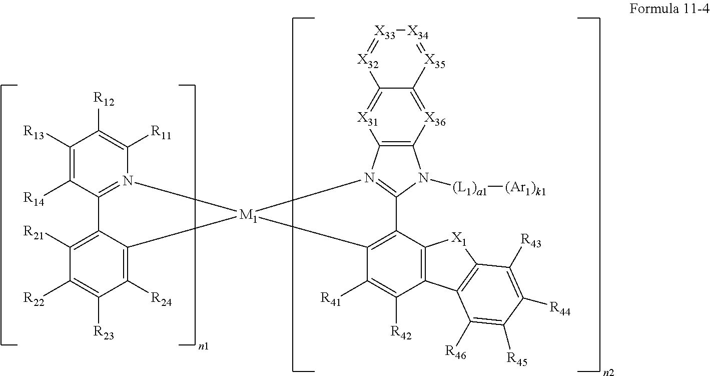

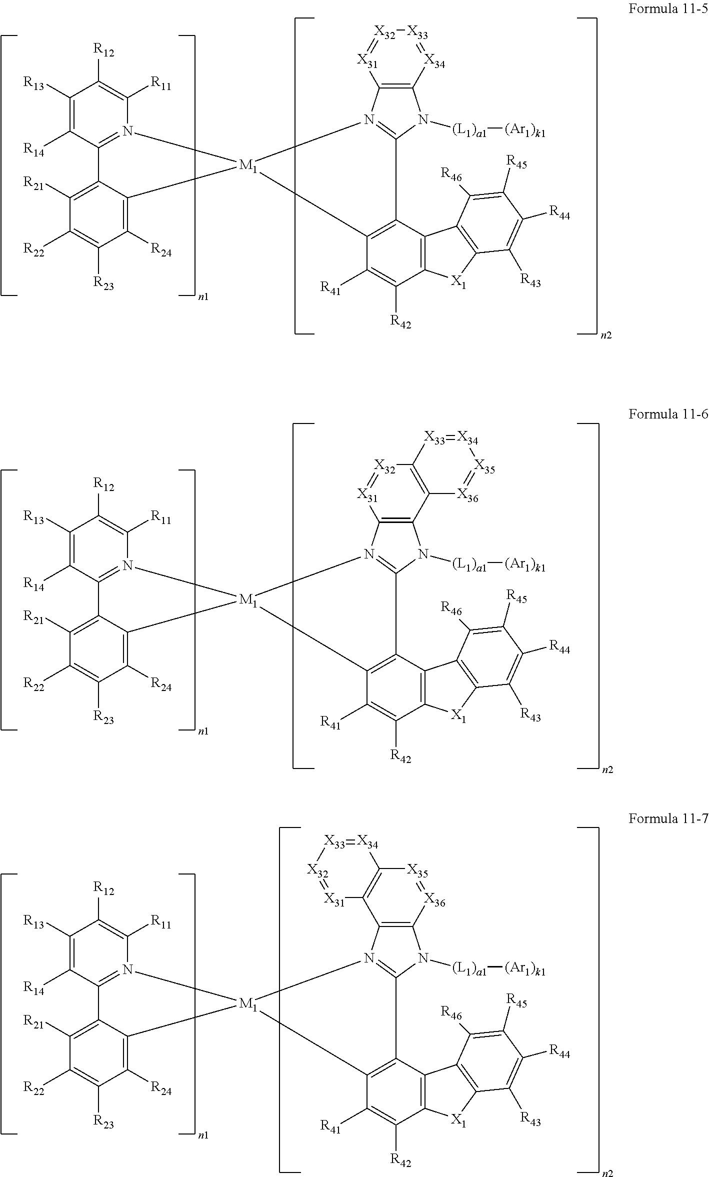

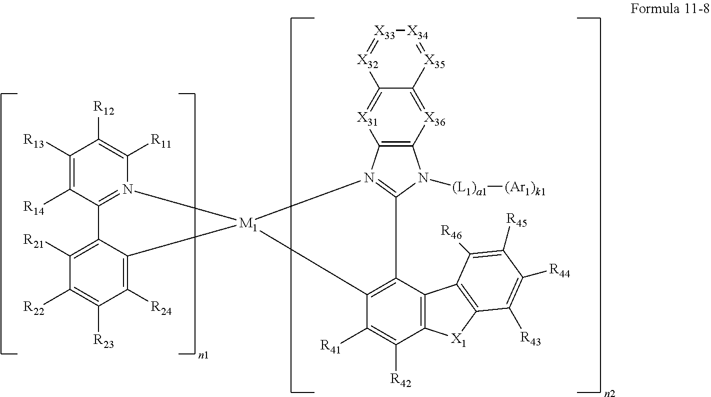

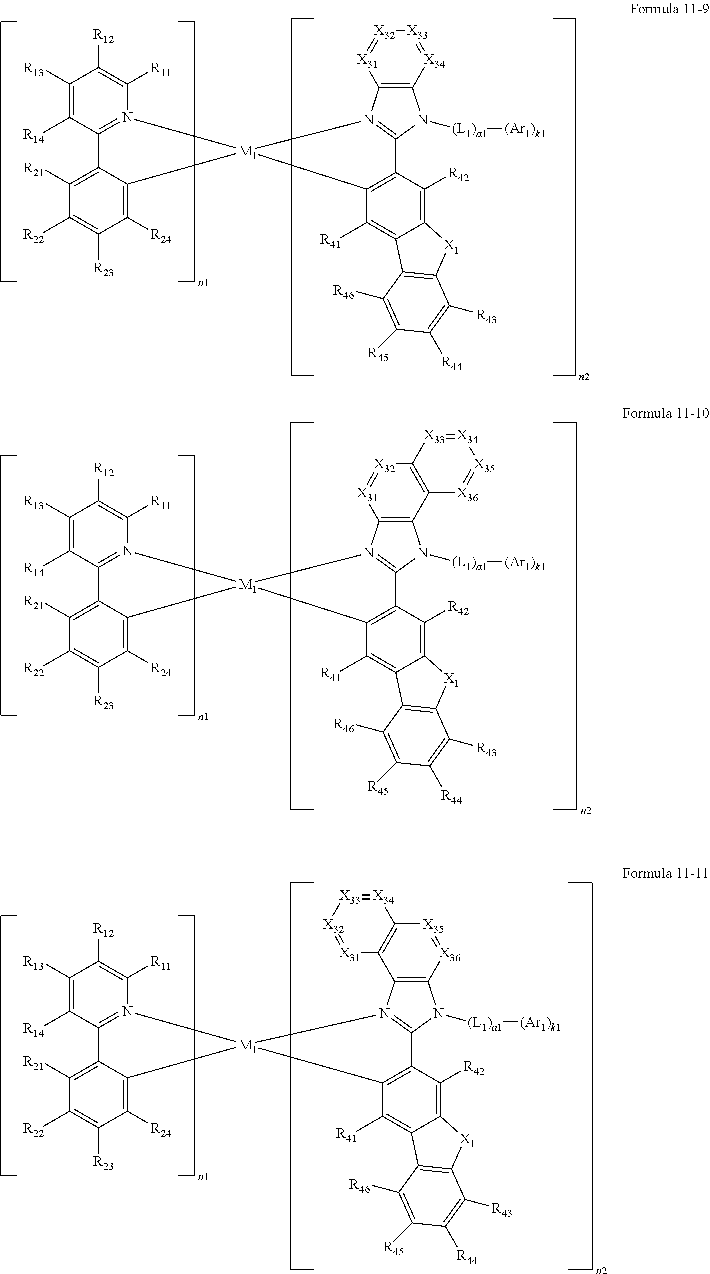

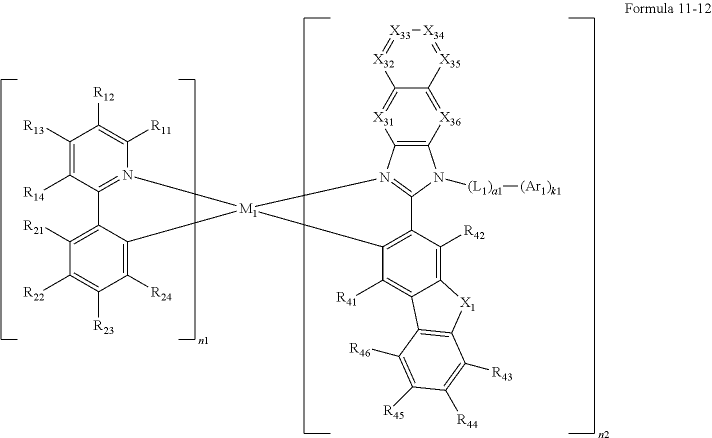

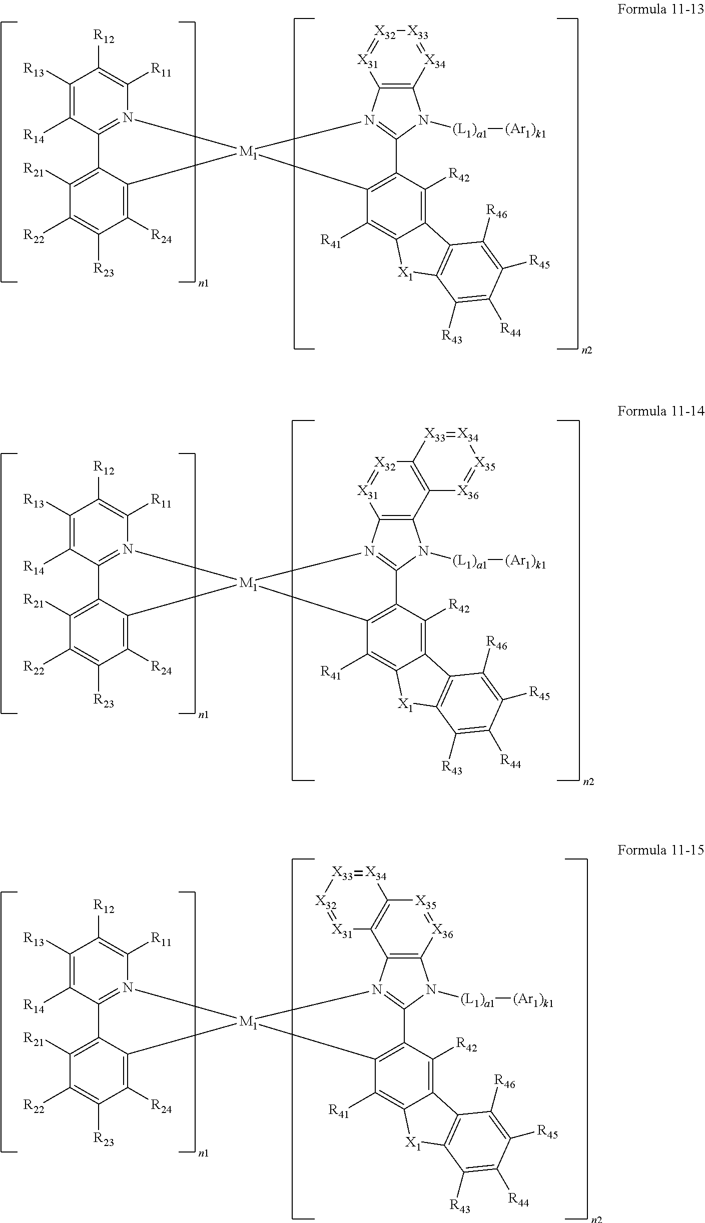

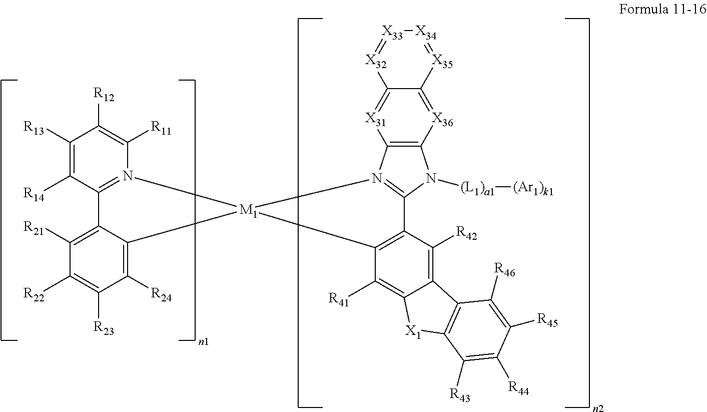

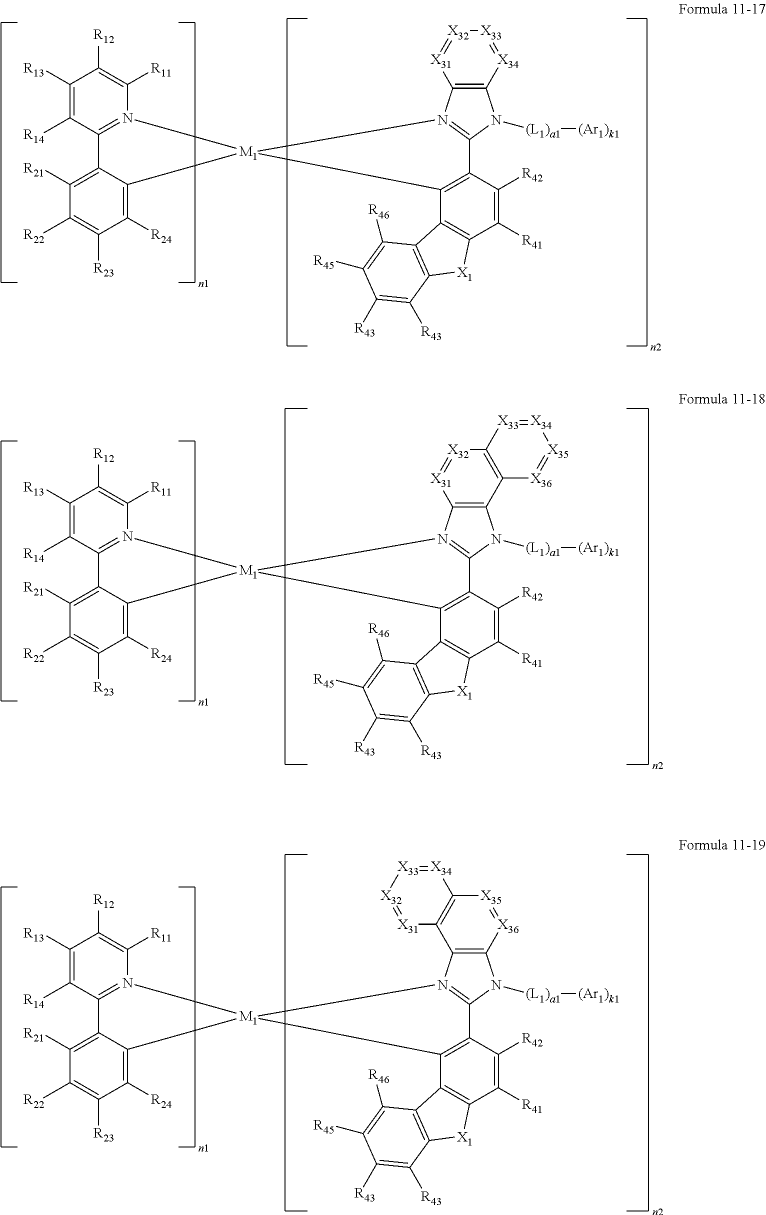

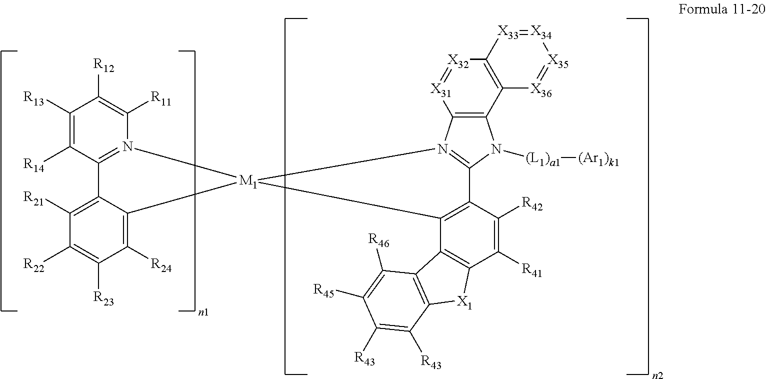

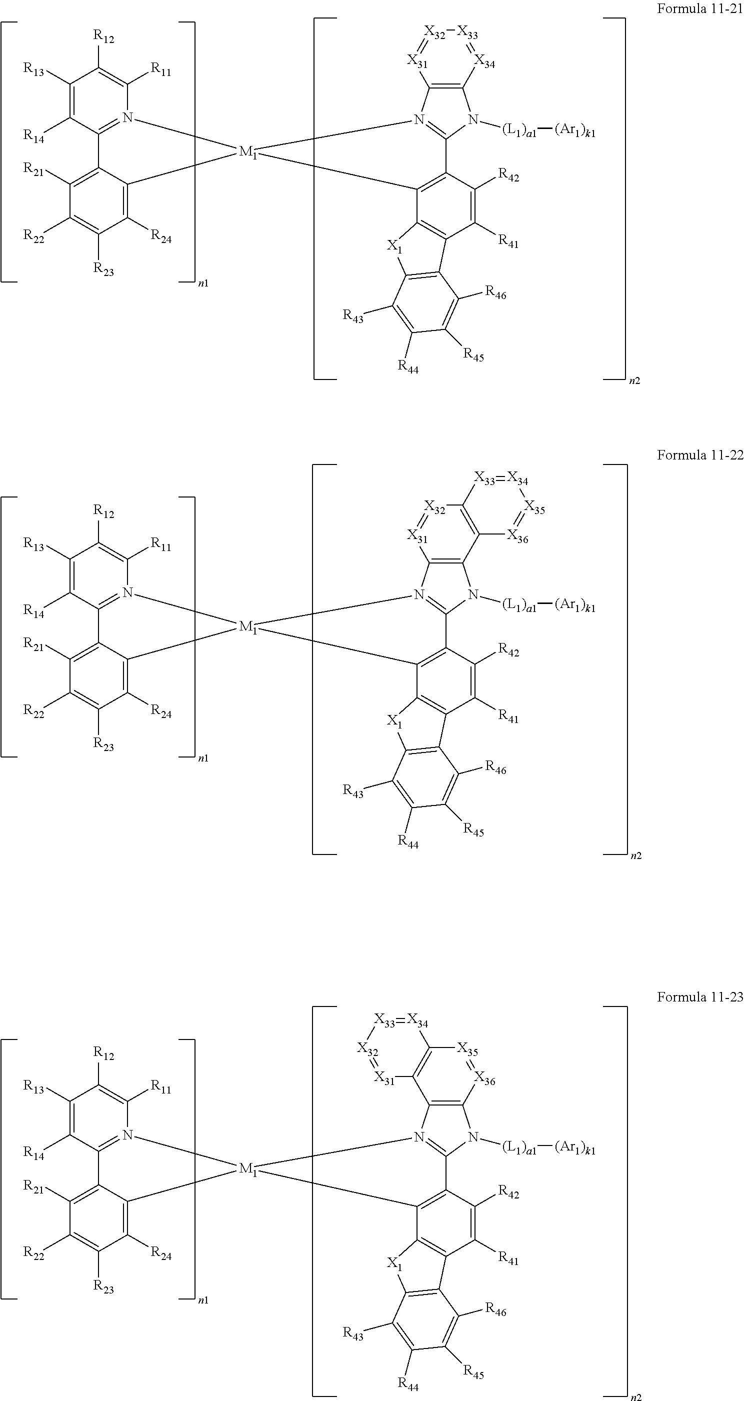

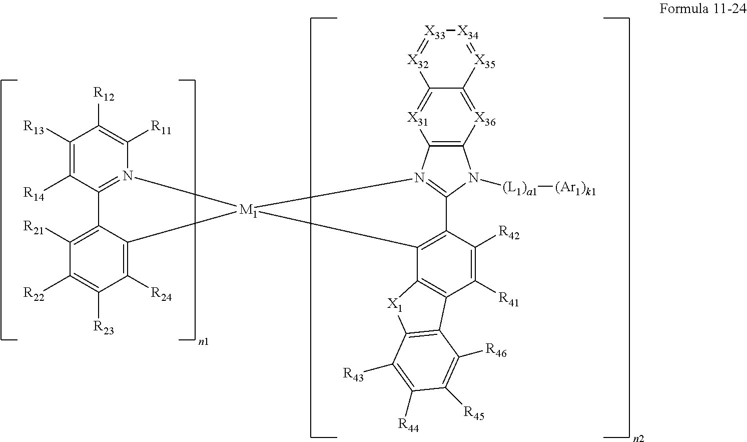

wherein, in Formulae 11-1 to 11-24,

M₁, n1, n2, X₁, L₁, a1, Ar₁, and k1 are each the same as described in the present specification,

X₃₁ may be C(R₃₁) or N, X₃₂ may be C(R₃₂) or N, X₃₃ may be C(R₃₃) or N, X₃₄ may be C(R₃₄) or N, X₃₅ may be C(R₃₅) or N, X₃₆ may be C(R₃₆) or N,

R₁₁ to R₁₄ are each independently the same as described in connection with R₁₀,

R₂₁ to R₂₄ are each independently the same as described in connection with R₂₀,

R₃₁ to R_{3M} are each independently the same as described in connection with R₃₀,

R₄₁ to R₄₆ are each independently the same as described in connection with R₄₀, and

at least one of Ar₁, R₁₁ to R₁₄, R₂₁ to R₂₄, R₃₁ to R₃₆, and R₄₁ to R₄₆ may include —F.

In one or more embodiments, two or more of R₃₁ to R₃₆ in Formulae 11-1 to 11-24 may be optionally linked to each other to form a C₅-C₃₀ carbocyclic group unsubstituted or substituted with at least one R₁₀ₐ or a C₁-C₃₀ heterocyclic group unsubstituted or substituted with at least one R₁₀ₐ. Examples of the “C₅-C₃₀ carbocyclic group unsubstituted or substituted with at least one R₁₀ₐ or a C₁-C₃₀ heterocyclic group unsubstituted or substituted with at least one R₁₀ₐ” as used herein include a benzene group, a naphthalene group, a cyclopentane group, a cyclopentadiene group, a cyclohexane group, a cycloheptane group, a bicyclo[2.2.1]heptane group, a furan group, a thiophene group, a pyrrole group, a silole group, an indene group, a benzofuran group, a benzothiophene group, an indole group, or a benzosilole group, each unsubstituted or substituted with at least one R₁₀ₐ. R₁₀ₐ is the same as described in connection with R₃₀. The C₅-C₃₀ carbocyclic group and the C₁-C₃₀ heterocyclic group are each the same as described in the present specification.

In one or more embodiments, at least one of R₁₁ to R₁₄, R₂₁ to R₂₄, R₃₁ to R₃₄, and R₄₁ to R₄₆ may include —F.

In one or more embodiments, at least one of R₂₁ to R₂₄ and R₄₁ to R₄₆ may include —F.

In one or more embodiments, at least one of R₁₀ in the number of b10 may be a substituted or unsubstituted C₁-C₆₀ alkyl group, —Si(Q₁)(Q₂)(Q₃), or —Ge(Q₁)(Q₂)(Q₃).

In one or more embodiments, at least one of R₁₀ in the number of b10 may be a methyl group, an ethyl group, an n-propyl group, an isopropyl group, an n-butyl group, an isobutyl group, a sec-butyl group, a tert-butyl group, an n-pentyl group, an isopentyl group, a 2-methylbutyl group, a sec-pentyl group, a tert-pentyl group, a neo-pentyl group, a 3-pentyl group, a 3-methyl-2-butyl group, a phenyl group, a biphenyl group, a C₁-C₂₀ alkylphenyl group, a naphthyl group, —Si(Q₁)(Q₂)(Q₃), or —Ge(Q₁)(Q₂)(Q₃).

In one or more embodiments, at least one of R₁₁ to R₁₄ in Formulae 11-1 to 11-24 may be a substituted or unsubstituted C₁-C₆₀ alkyl group, —Si(Q₁)(Q₂)(Q₃), or —Ge(Q₁)(Q₂)(Q₃).

In one or more embodiments, at least one of R₁₁ to R₁₄ in Formulae 11-1 to 11-24 may be a methyl group, an ethyl group, an n-propyl group, an isopropyl group, an n-butyl group, an isobutyl group, a sec-butyl group, a tert-butyl group, an n-pentyl group, an isopentyl group, a 2-methylbutyl group, a sec-pentyl group, a tert-pentyl group, a neo-pentyl group, a 3-pentyl group, a 3-methyl-2-butyl group, a phenyl group, a biphenyl group, a C₁-C₂₀ alkylphenyl group, a naphthyl group, —Si(Q₁)(Q₂)(Q₃), or —Ge(Q₁)(Q₂)(Q₃).

In one or more embodiments, at least one of R₁₁ to R₁₄ in Formulae 1-1 to 1-16 may be a substituted or unsubstituted C₁-C₆₀ alkyl group, —Si(Q₁)(Q₂)(Q₃), or —Ge(Q₁)(Q₂)(Q₃).

In one or more embodiments, at least one of R₁₁ to R₁₄ in Formulae 1-1 to 1-16 may be a methyl group, an ethyl group, an n-propyl group, an isopropyl group, an n-butyl group, an isobutyl group, a sec-butyl group, a tert-butyl group, an n-pentyl group, an isopentyl group, a 2-methylbutyl group, a sec-pentyl group, a tert-pentyl group, a neo-pentyl group, a 3-pentyl group, a 3-methyl-2-butyl group, a phenyl group, a biphenyl group, a C₁-C₂₀ alkylphenyl group, a naphthyl group, —Si(Q₁)(Q₂)(Q₃), or —Ge(Q₁)(Q₂)(Q₃).

In one or more embodiments, at least one of R₁₀ in the number of b10 may include deuterium, and at least one of R₂₀ in the number of b20 may include deuterium.

In one or more embodiments, at least one of R₁₁ to R₁₄ and R₂₁ to R₂₄ in Formulae 11-1 to 11-24 may include deuterium.

In one or more embodiments, at least one of R₁₁ to R₁₄ and R₂₁ to R₂₄ in Formulae 1-1 to 1-16 and 2-1 to 2-16 may include deuterium.

In one or more embodiments, Ln₁ may be a ligand represented by one of Formulae A1 to A285:

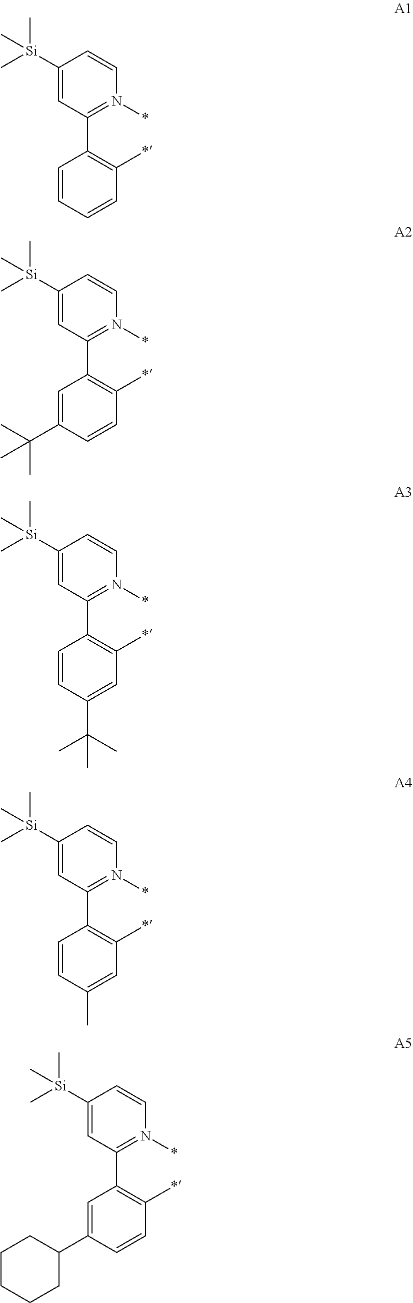

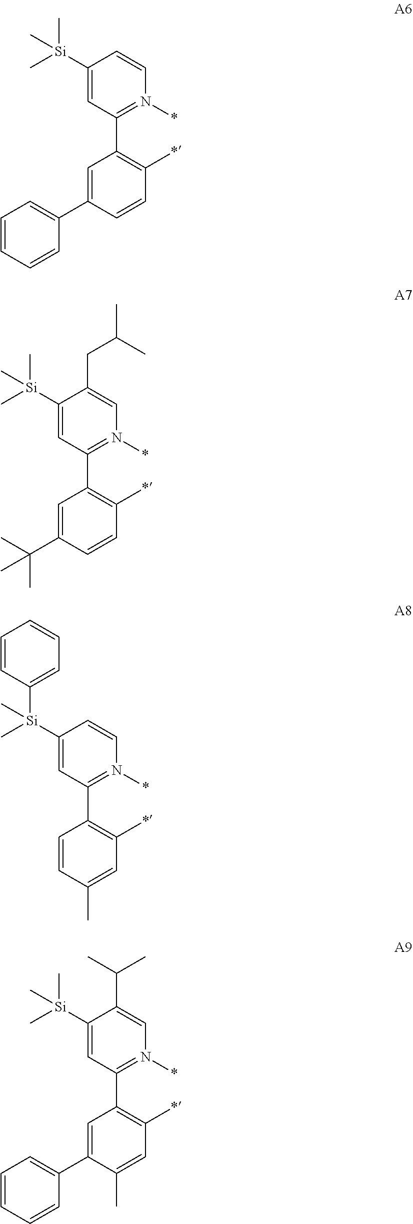

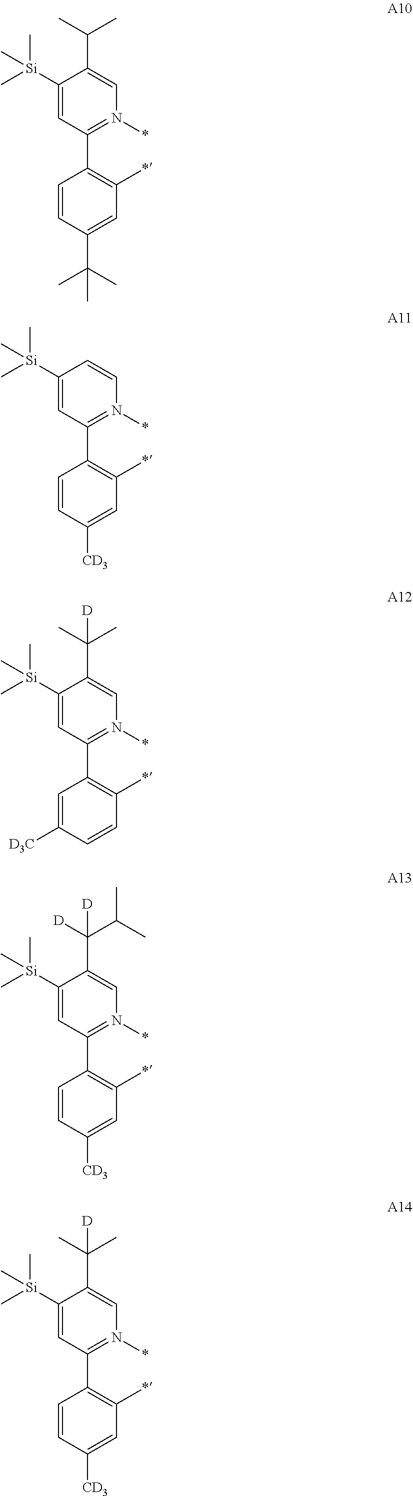

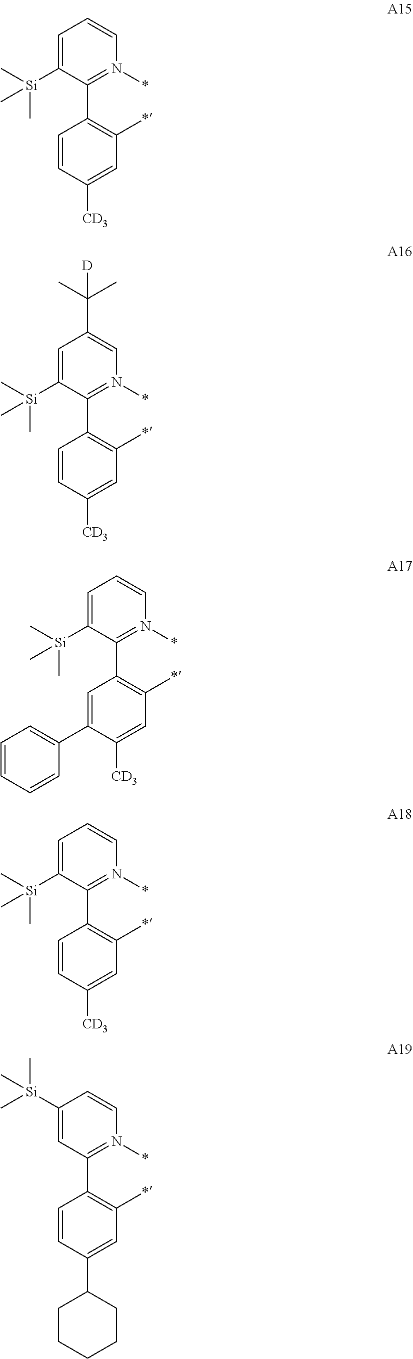

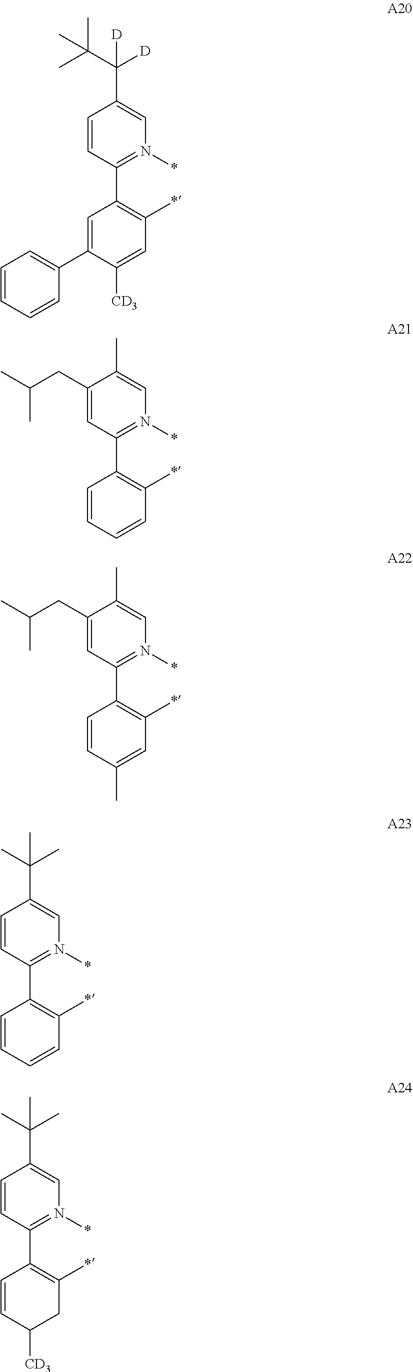

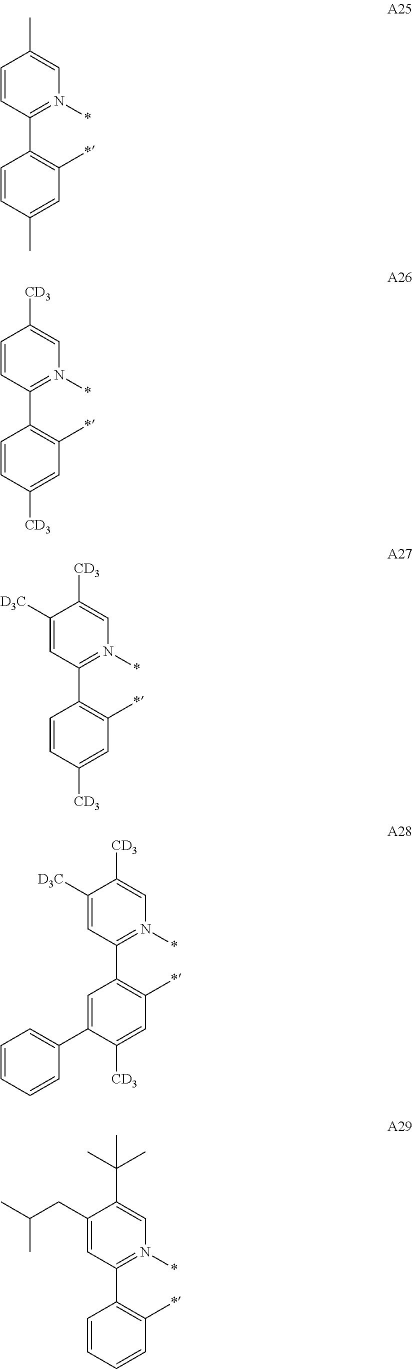

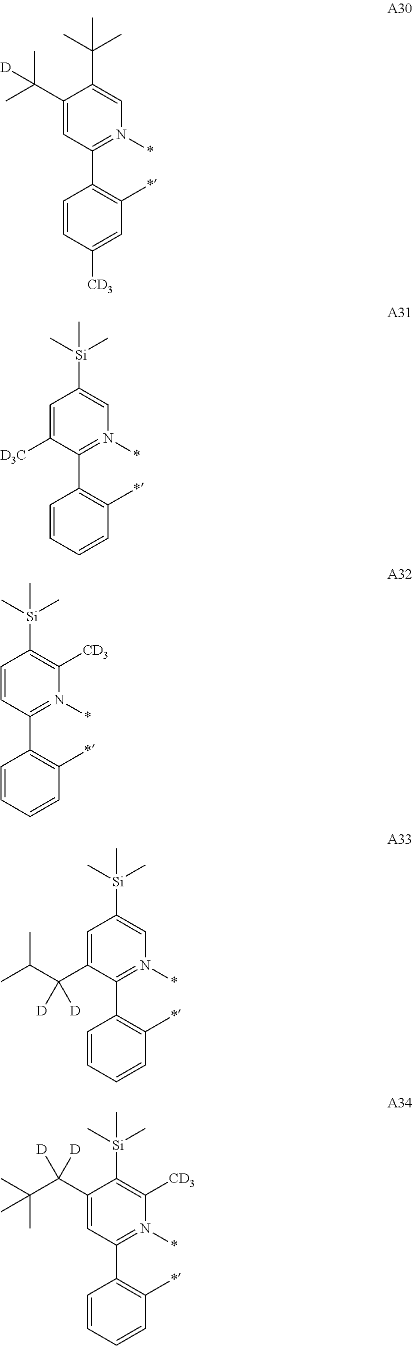

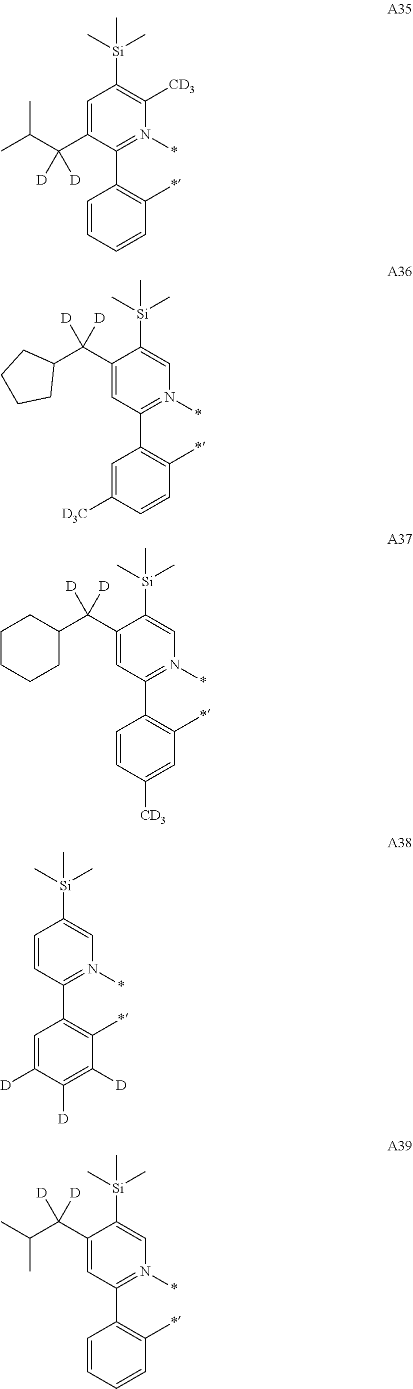

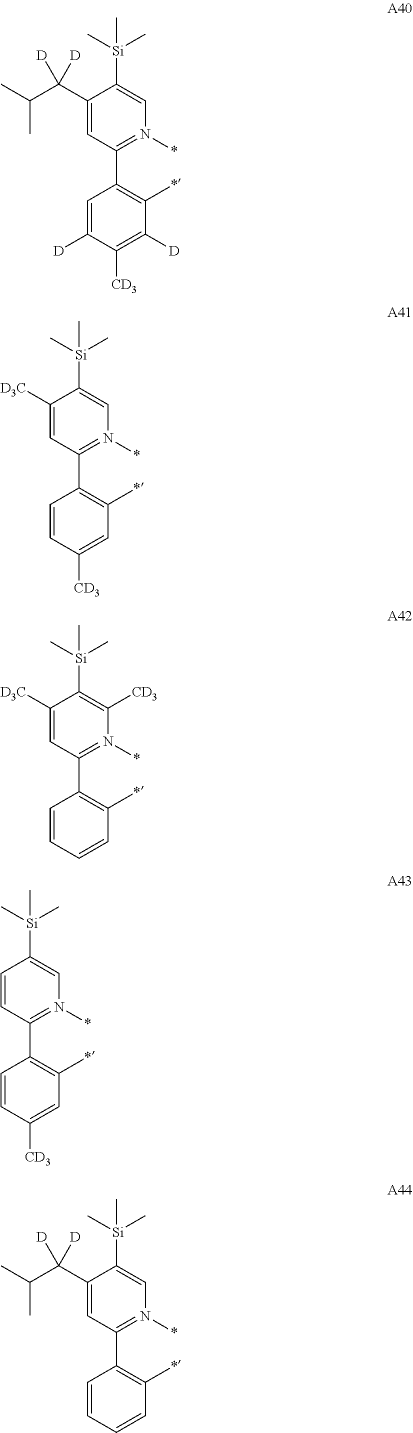

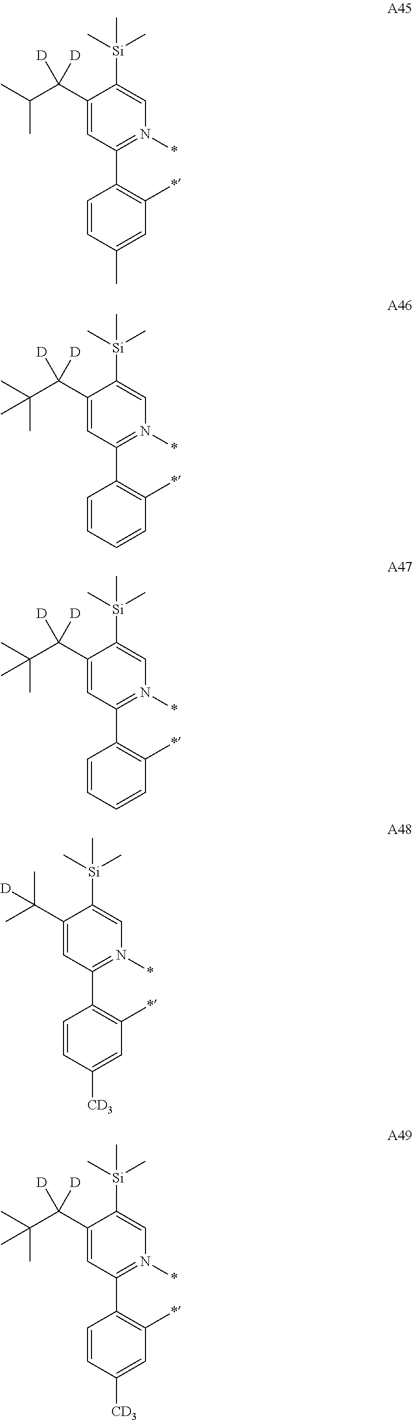

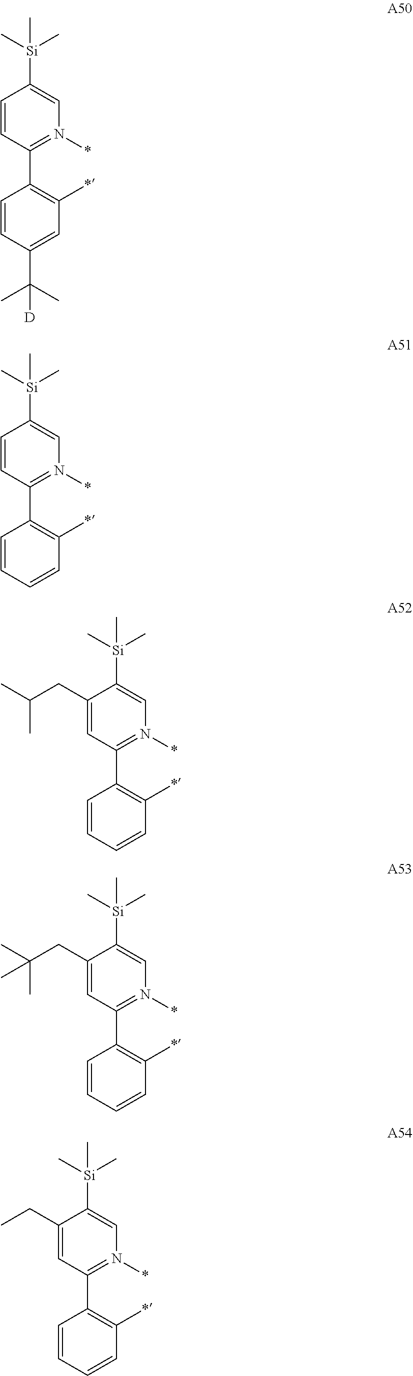

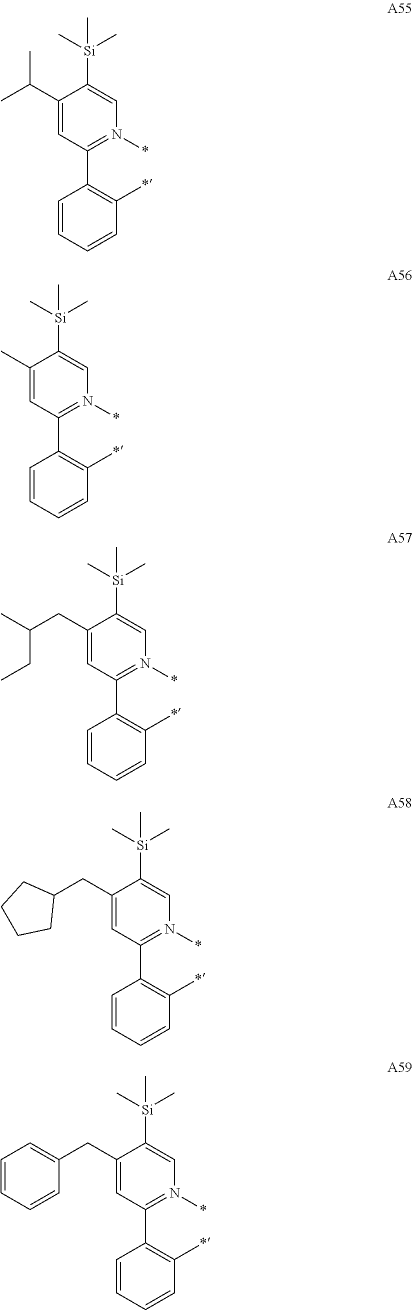

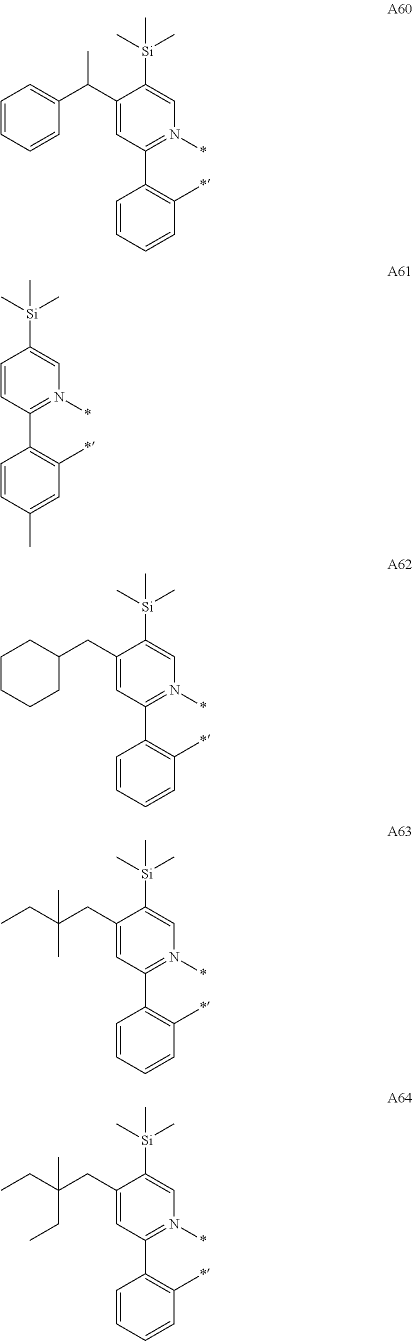

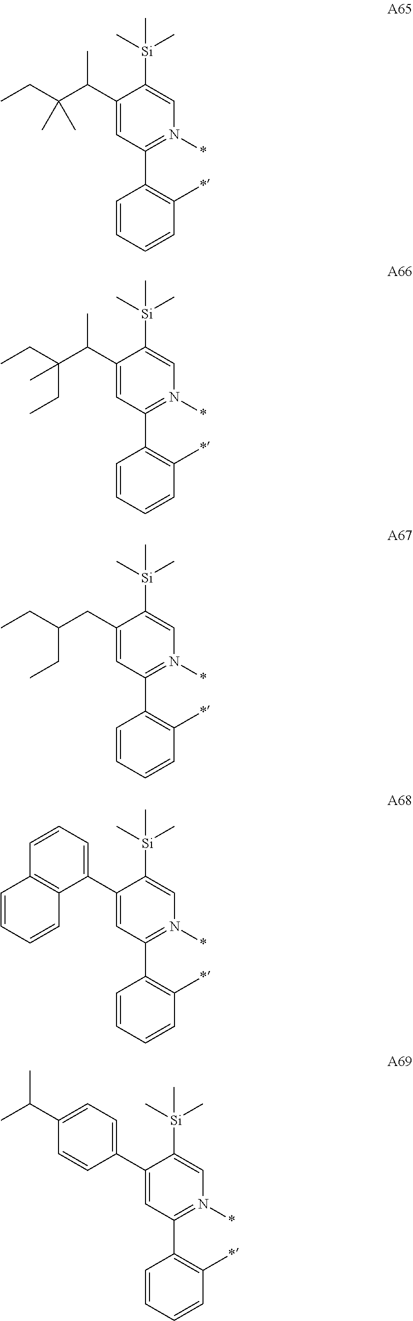

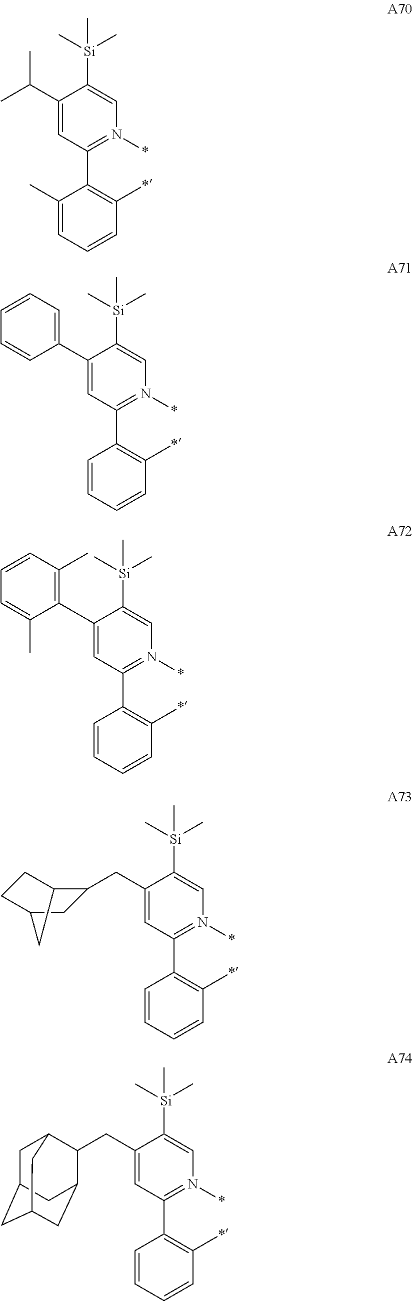

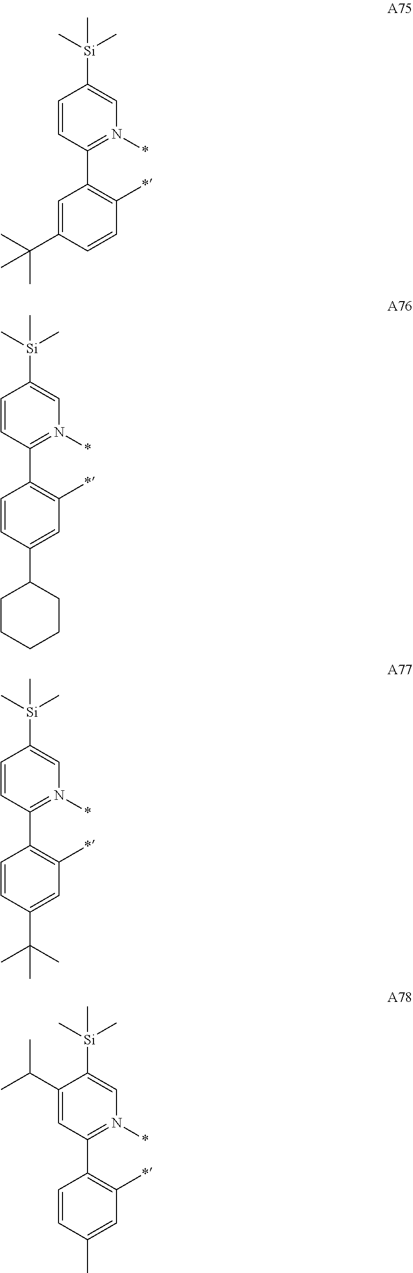

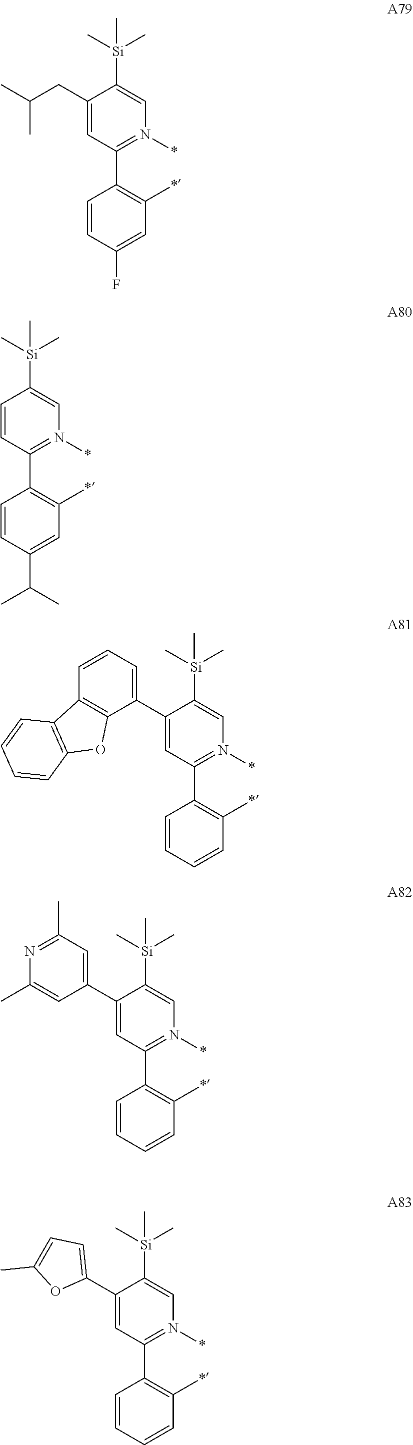

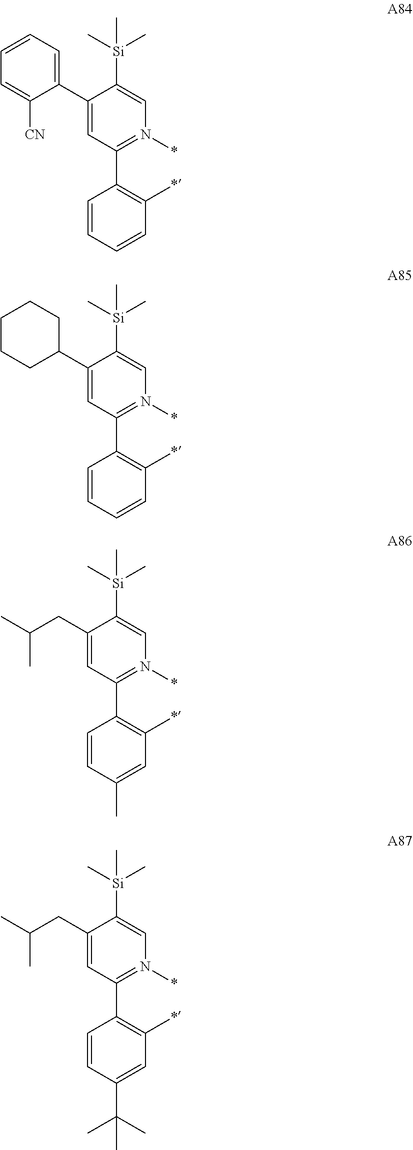

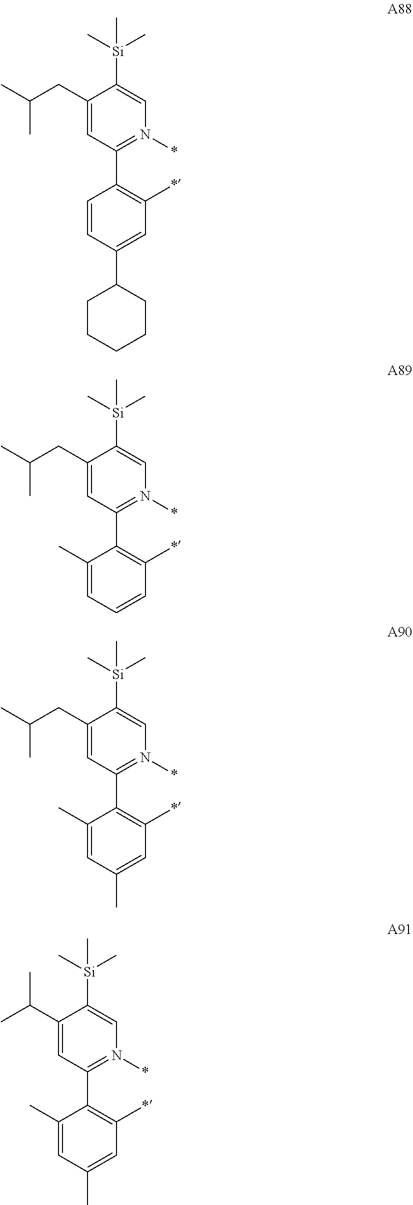

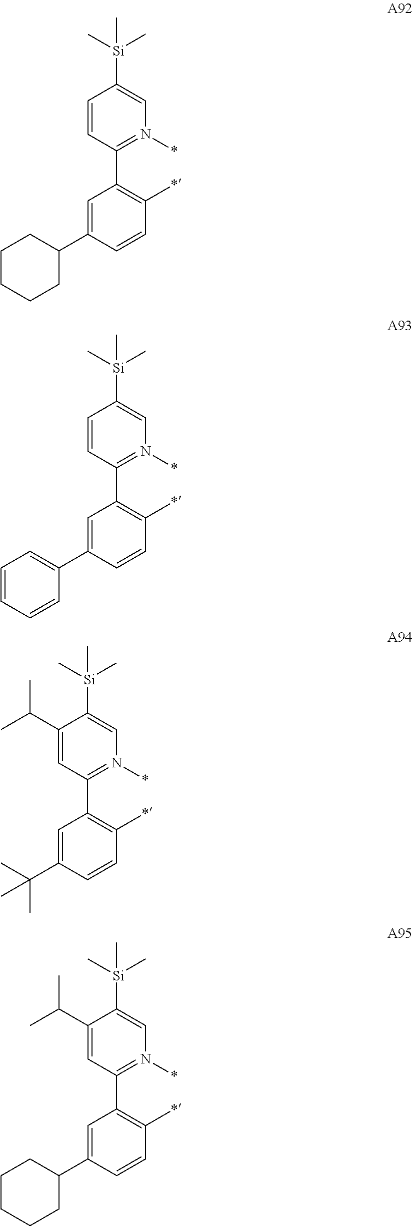

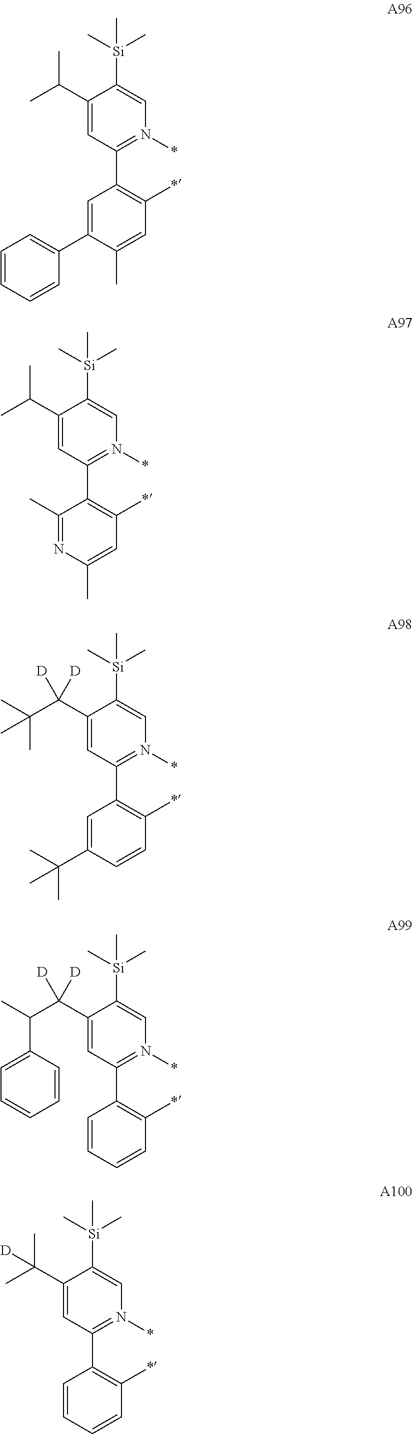

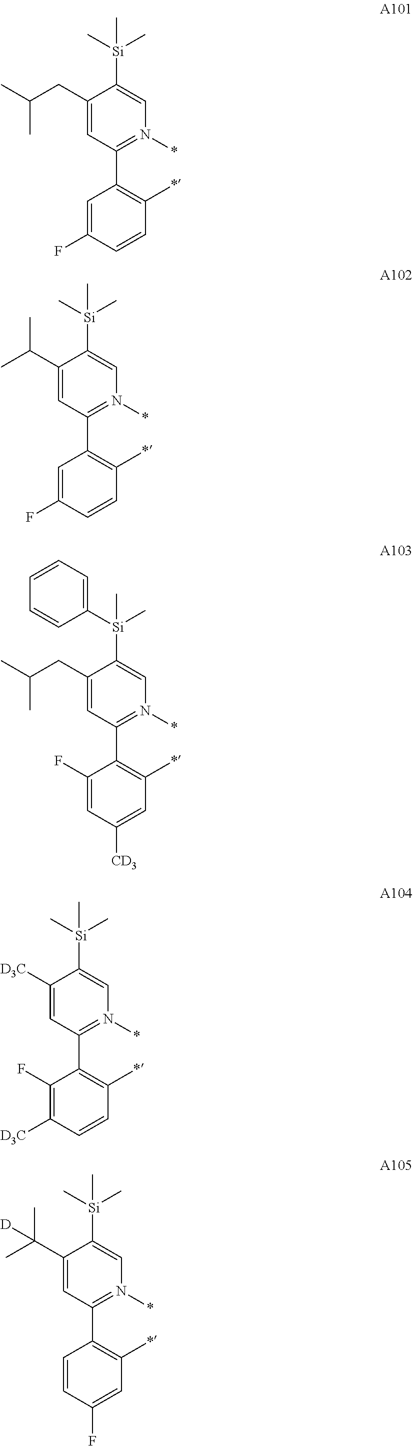

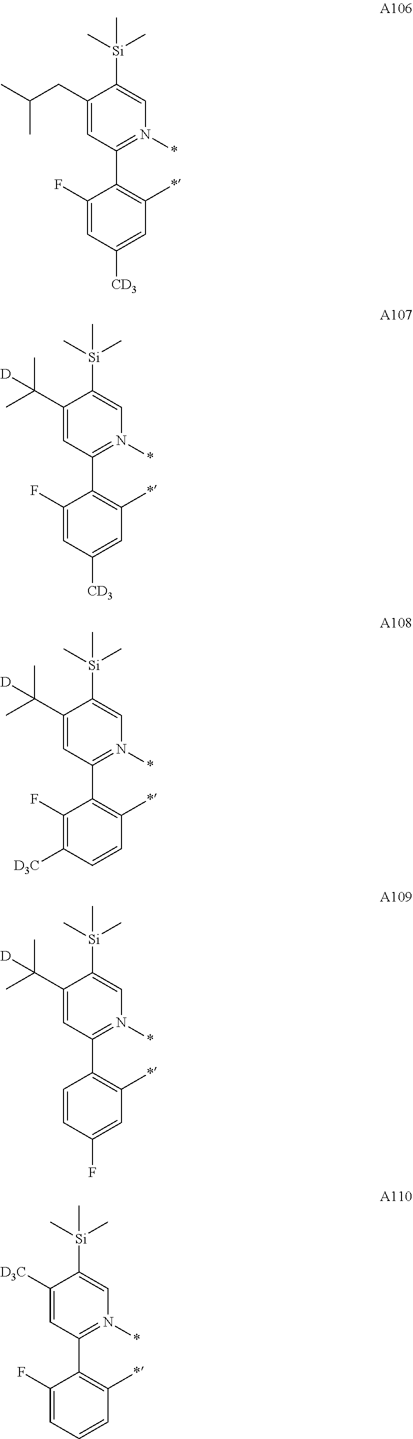

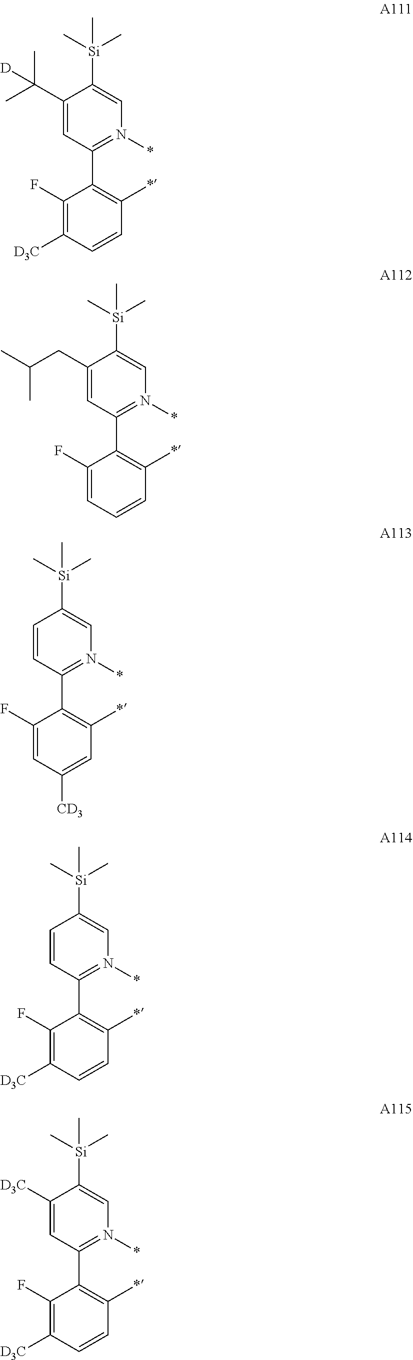

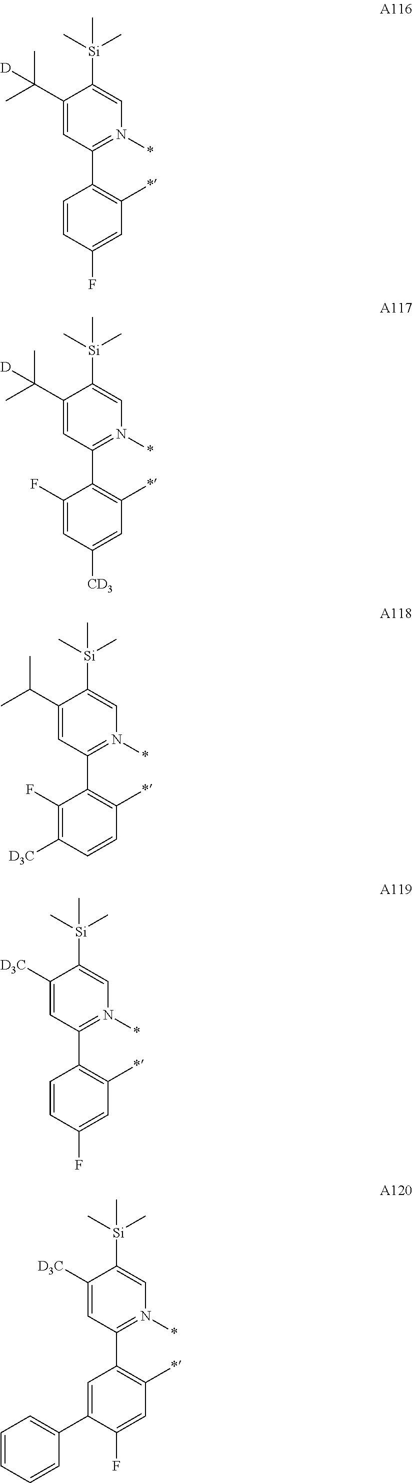

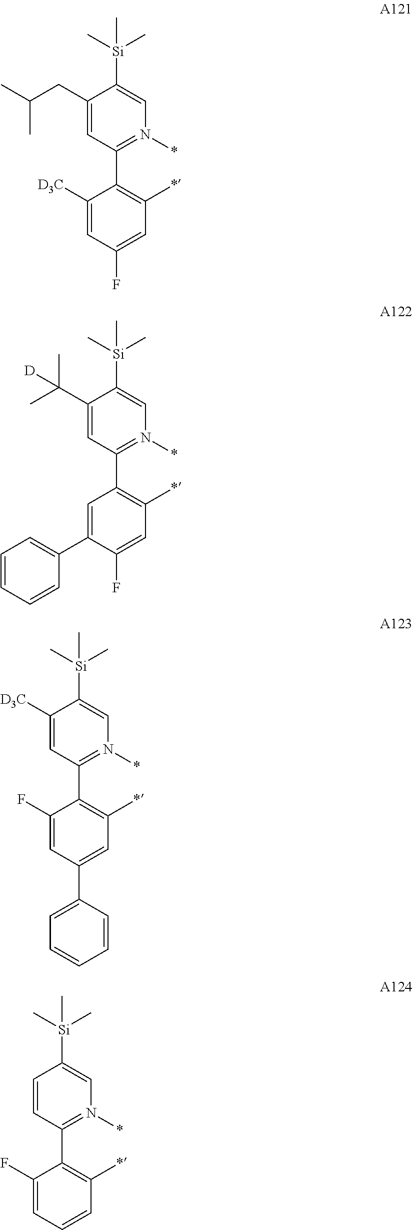

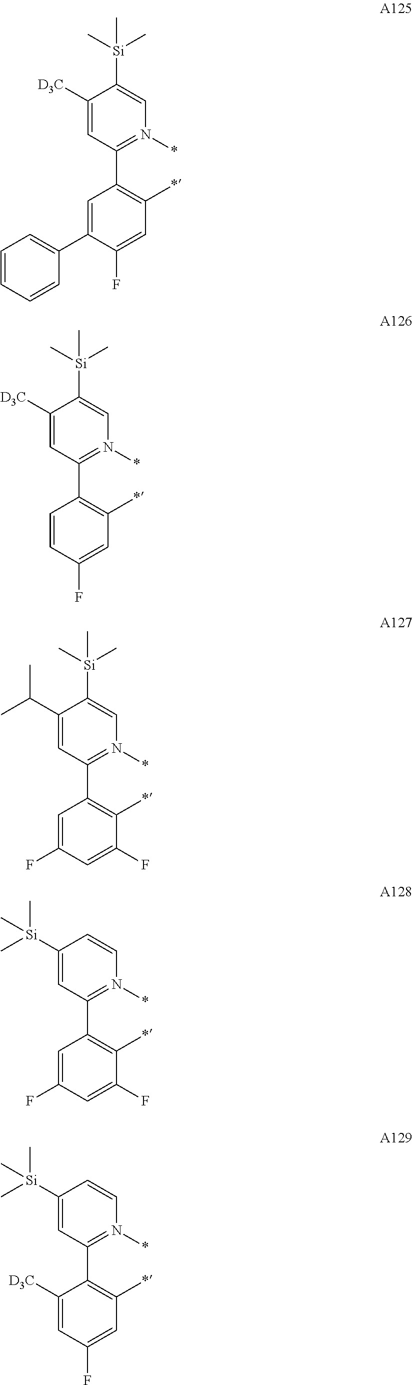

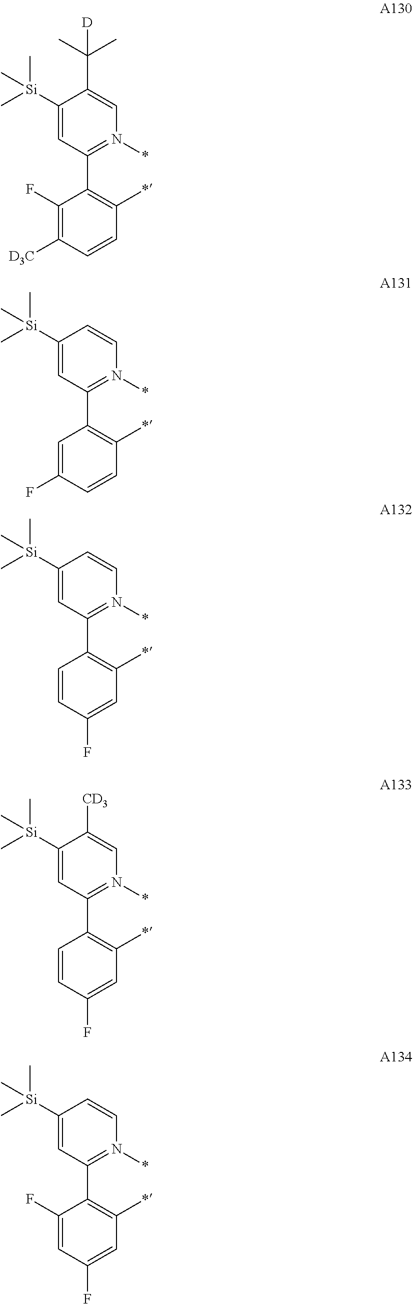

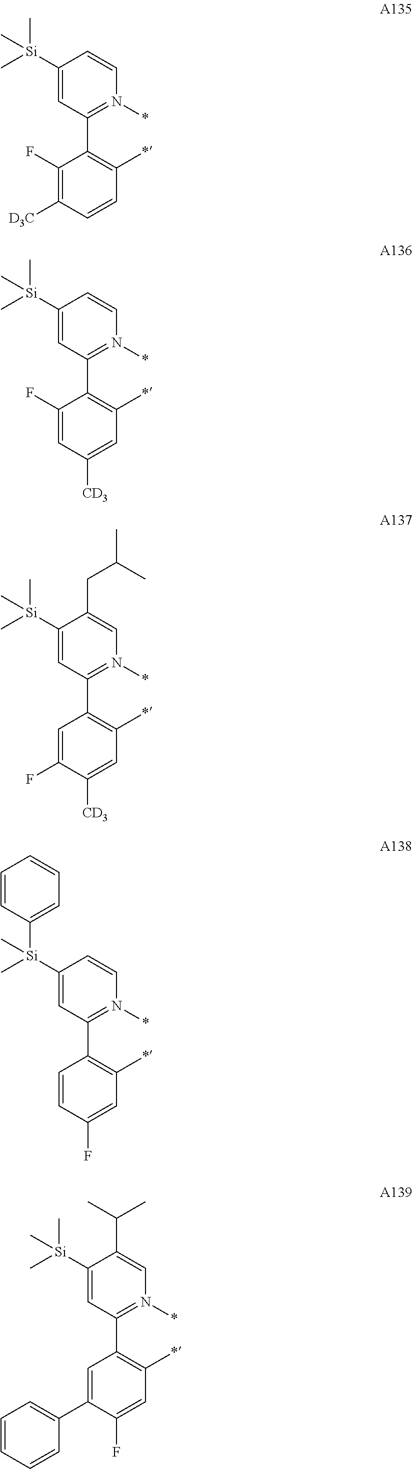

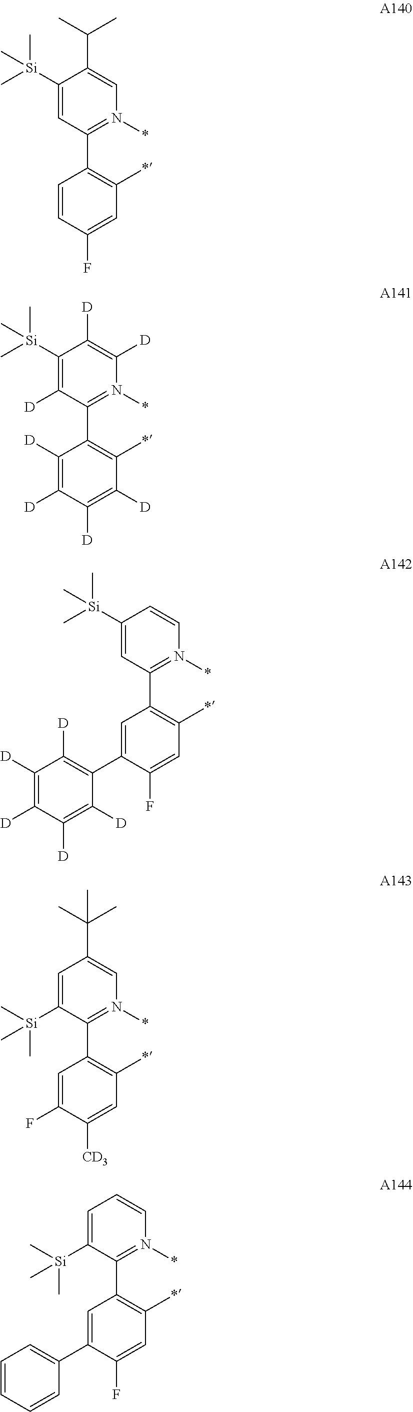

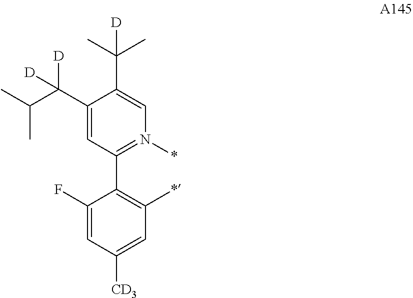

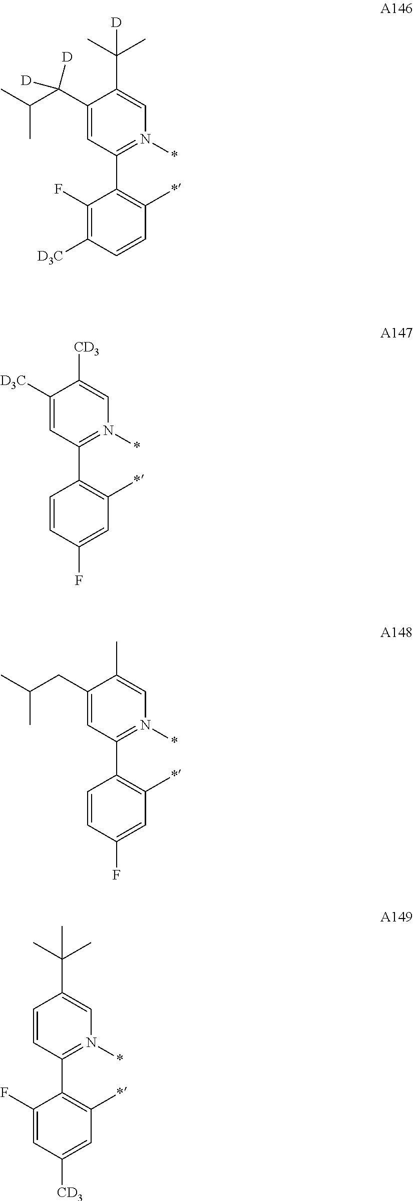

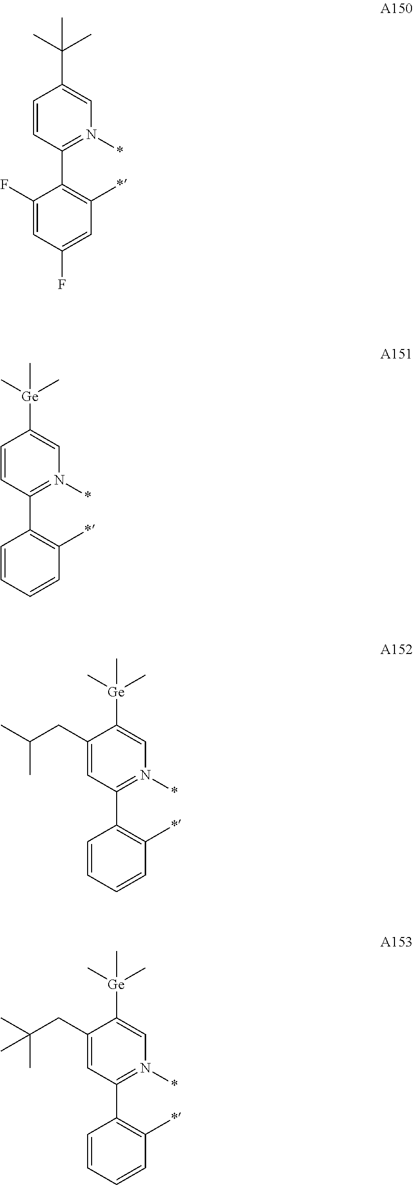

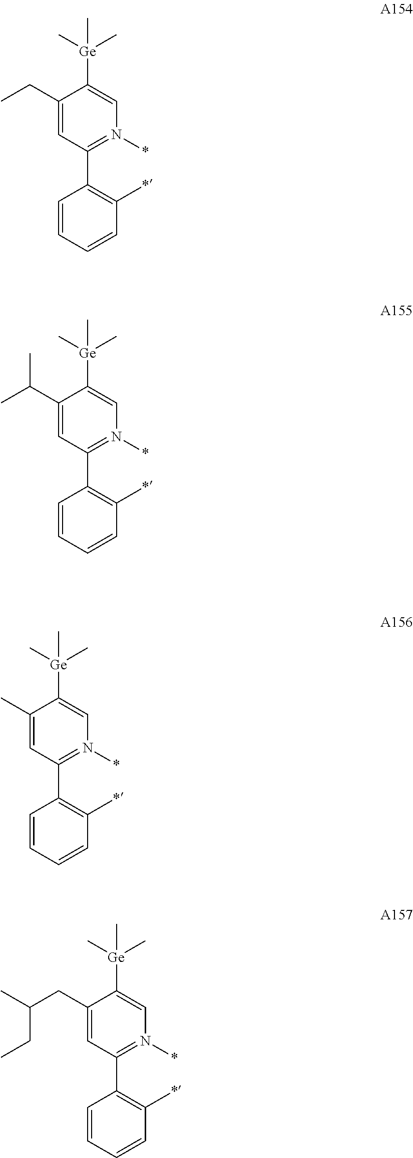

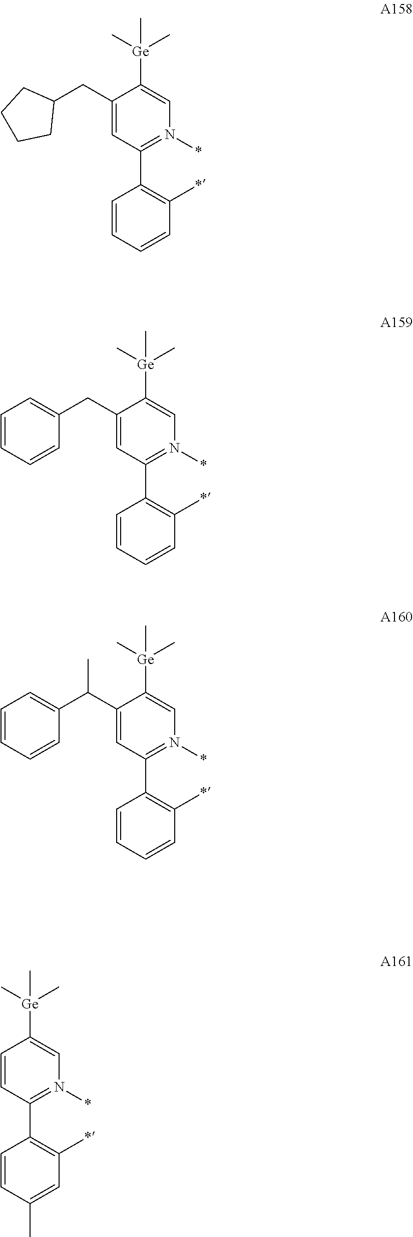

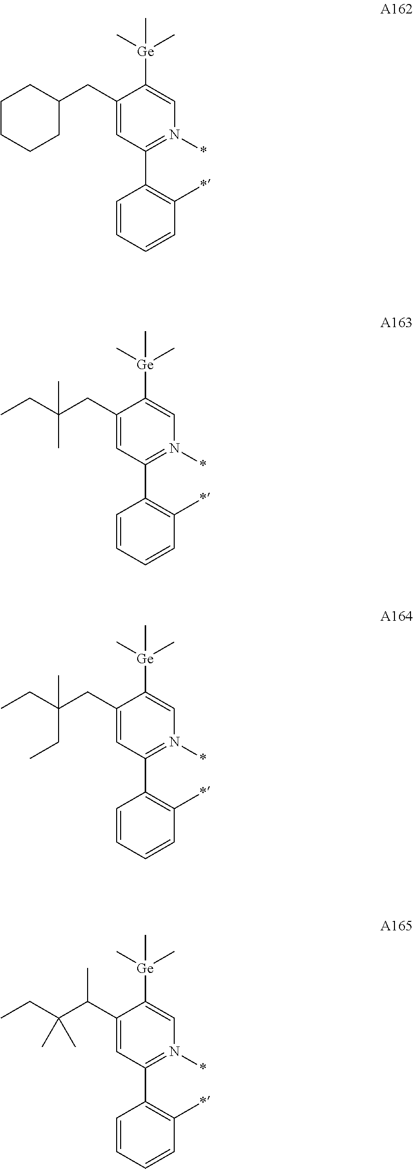

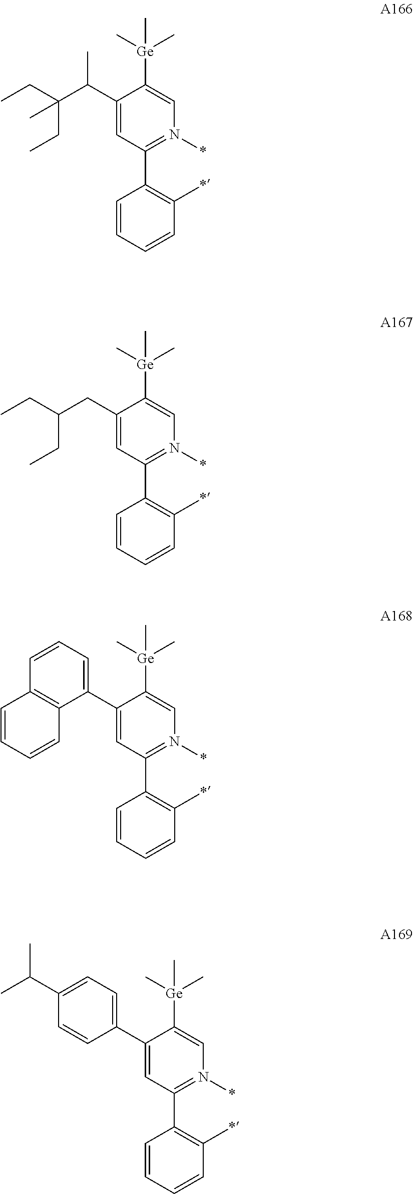

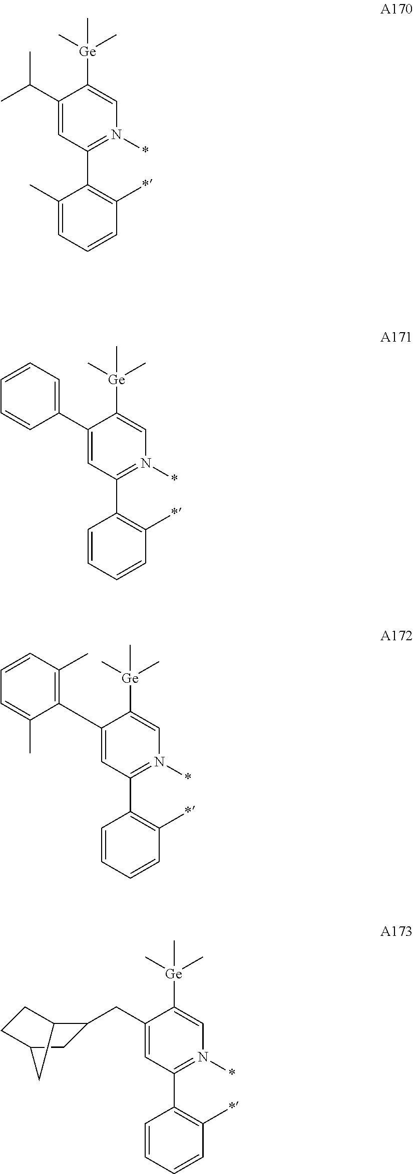

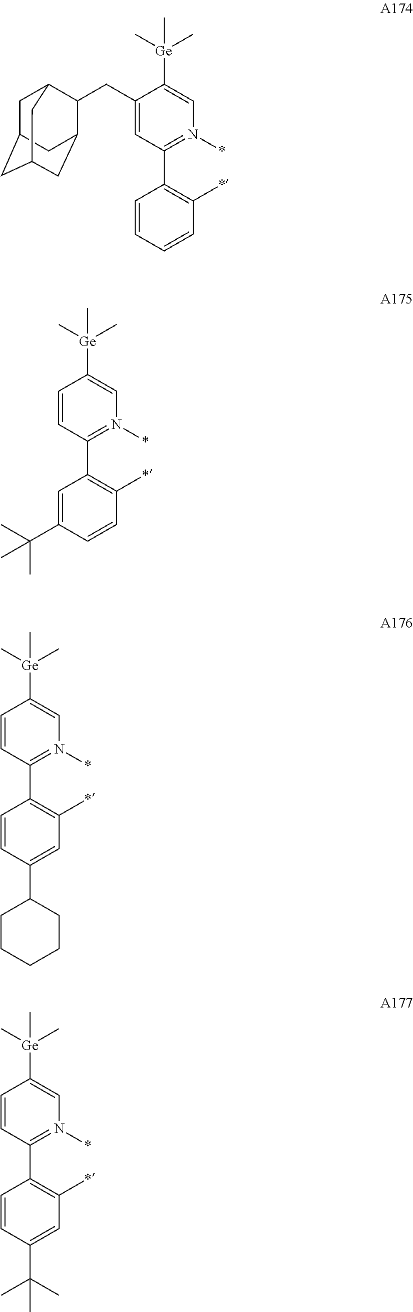

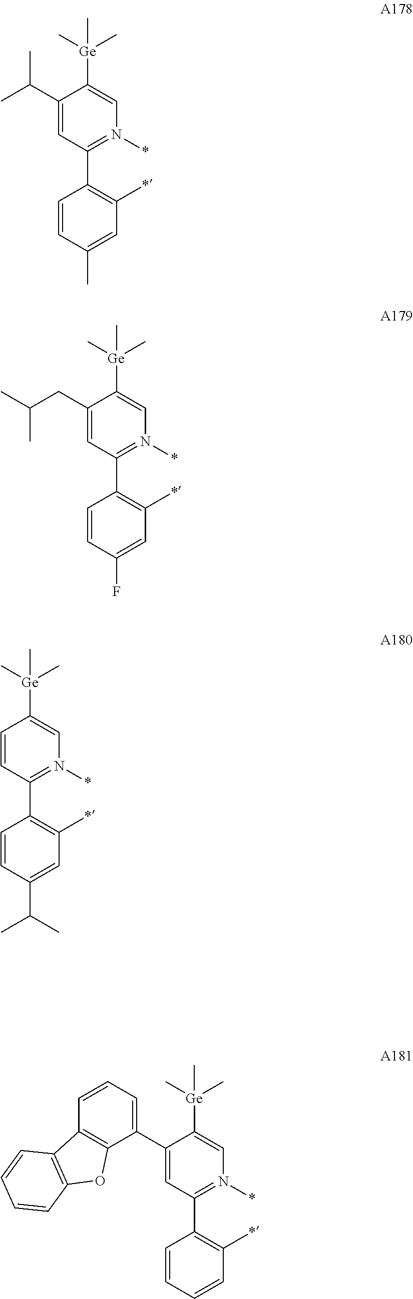

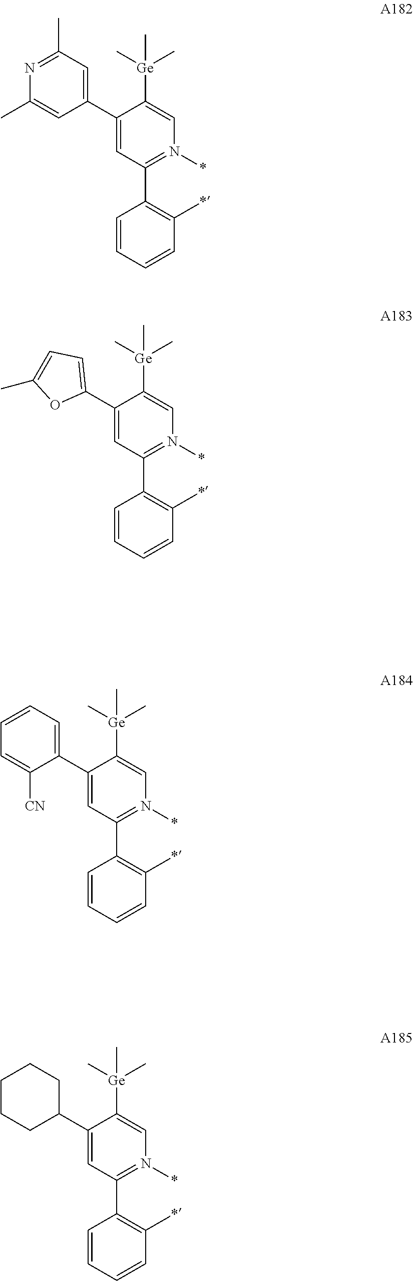

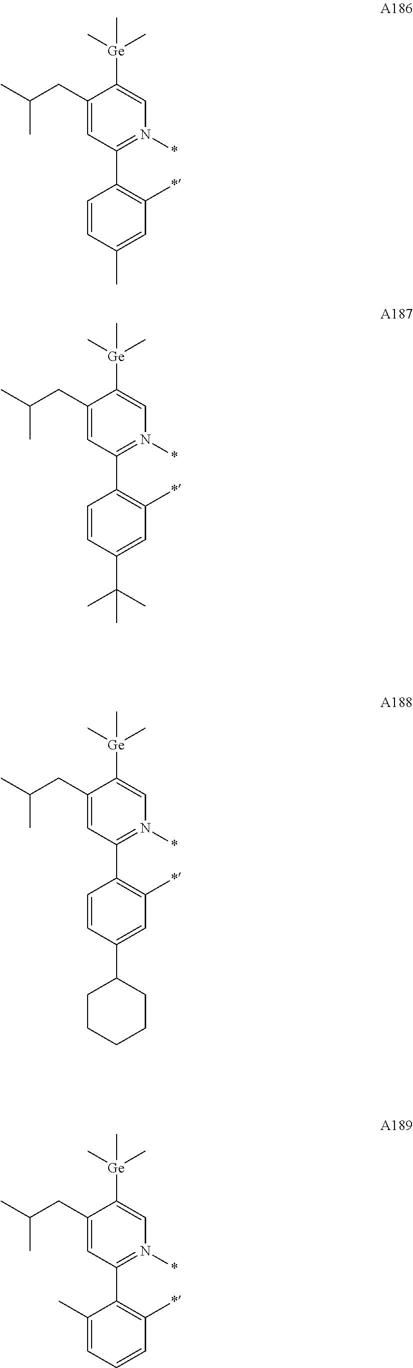

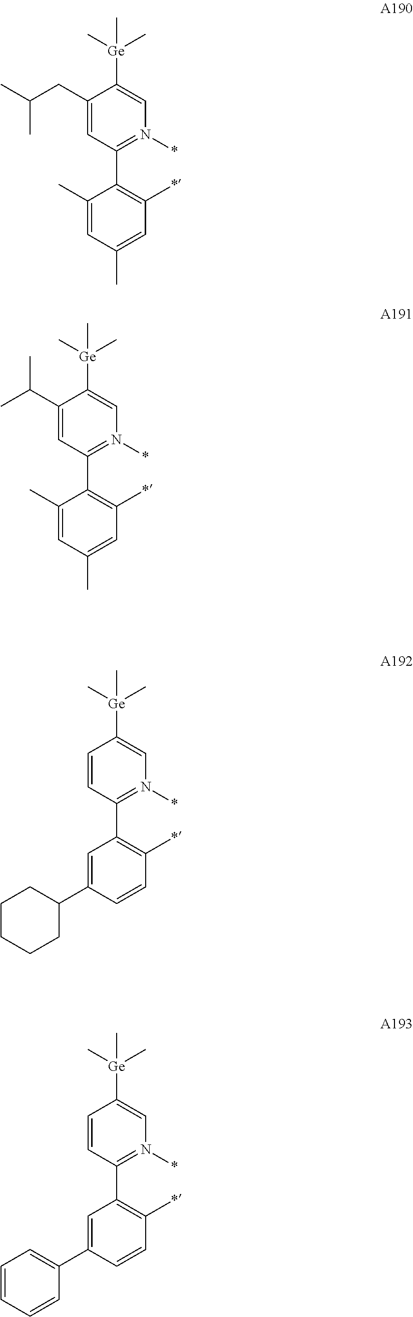

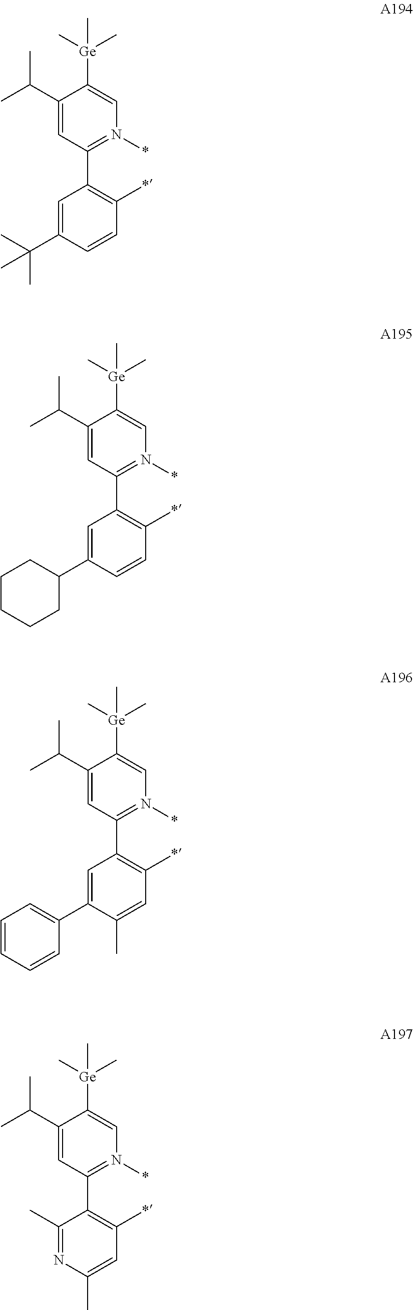

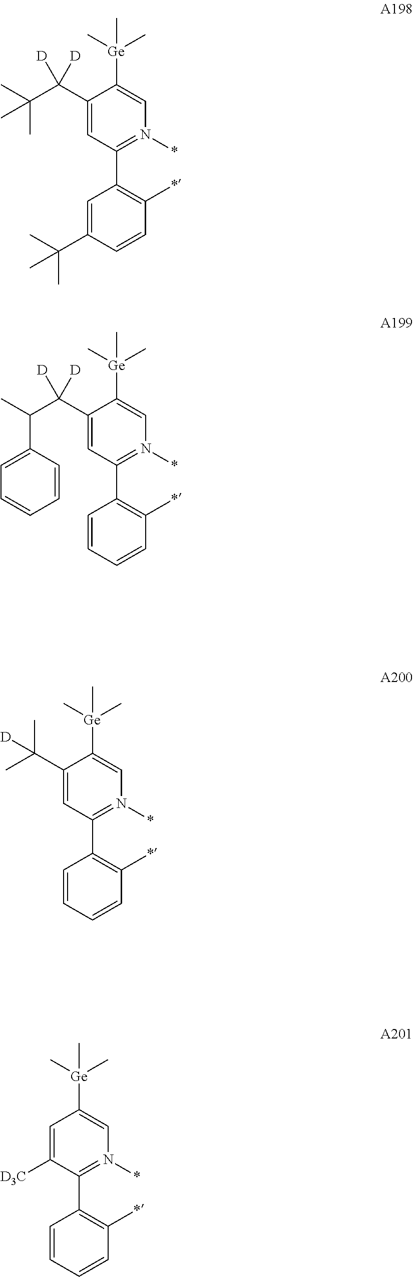

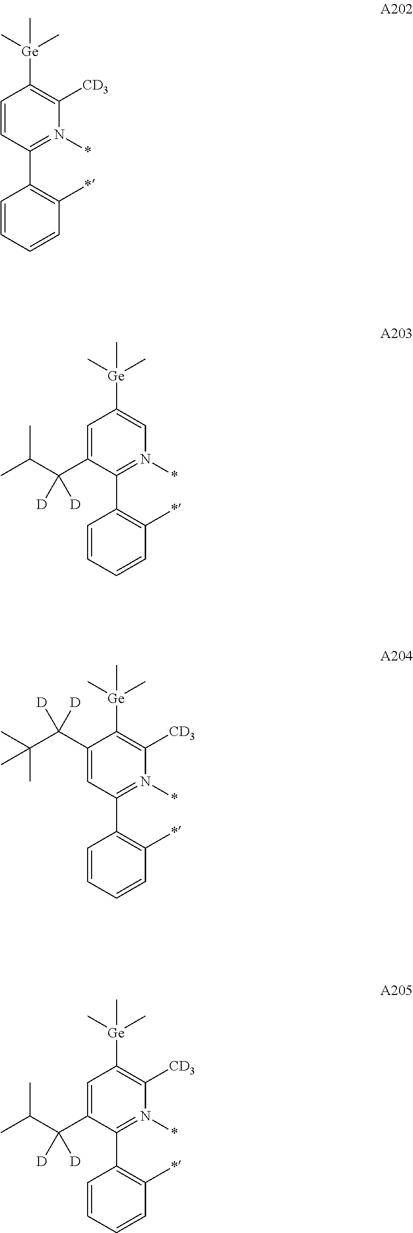

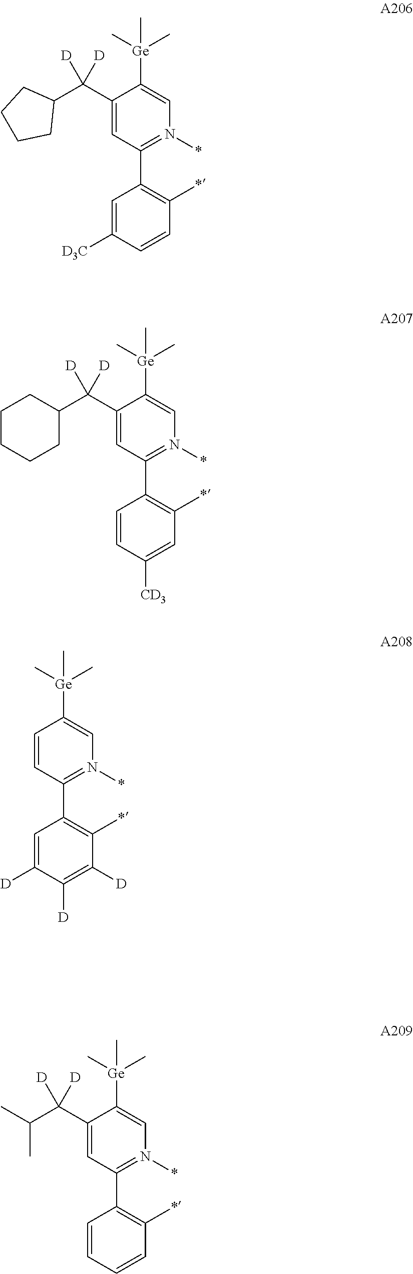

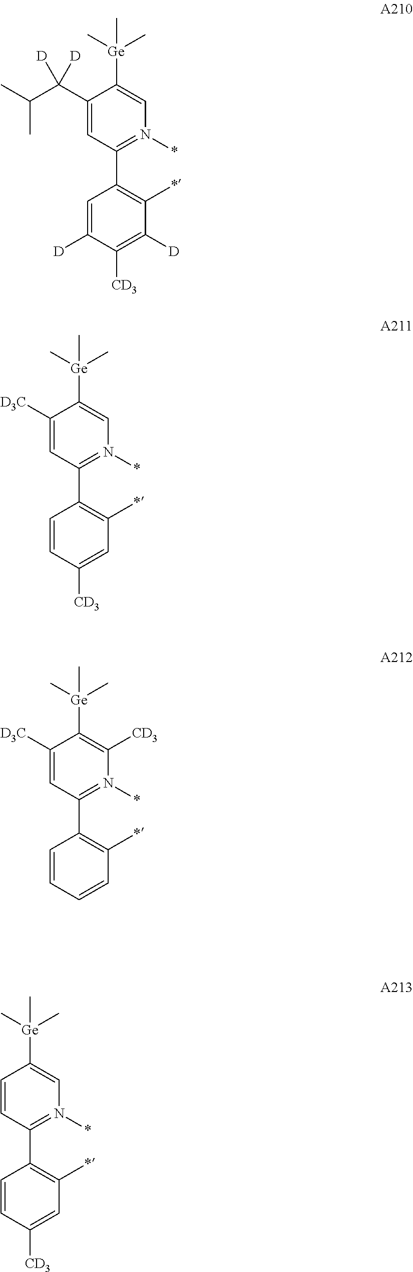

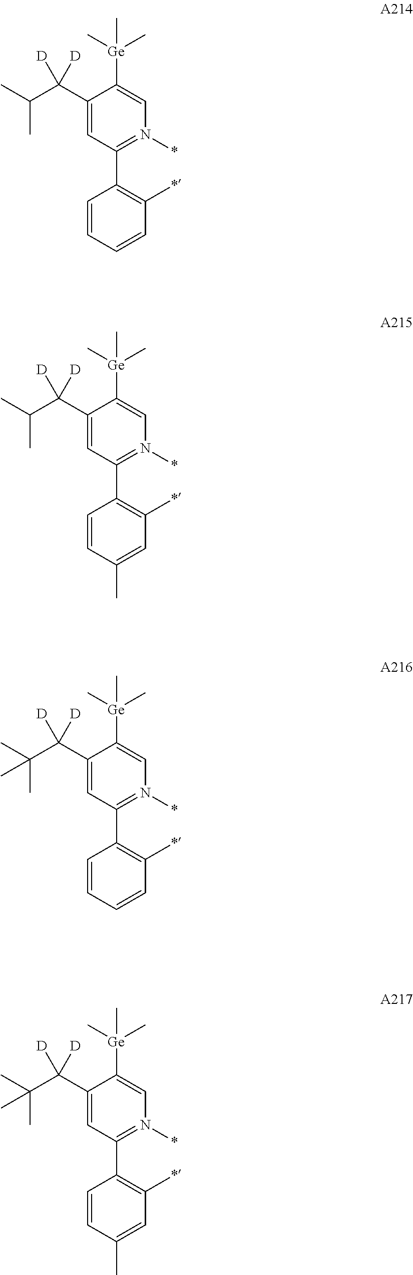

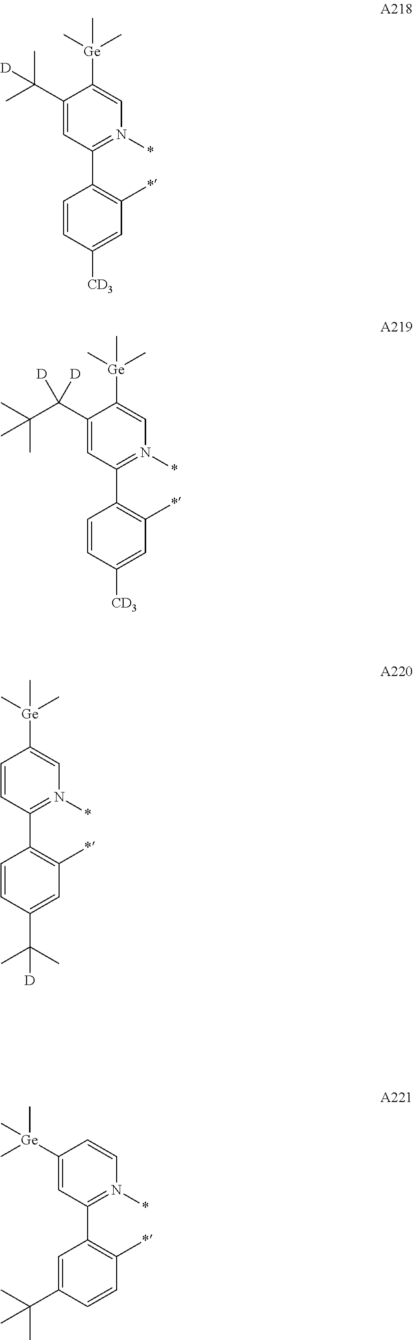

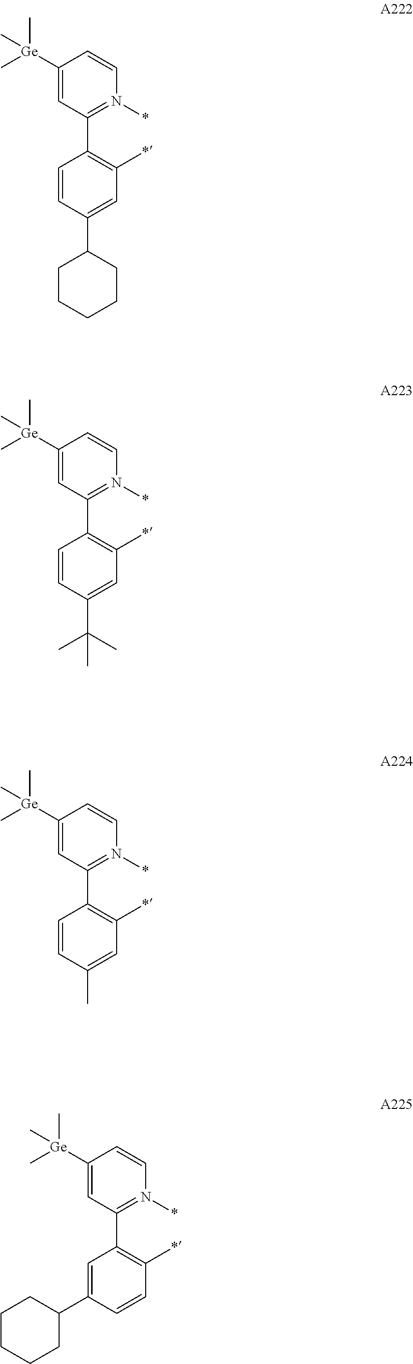

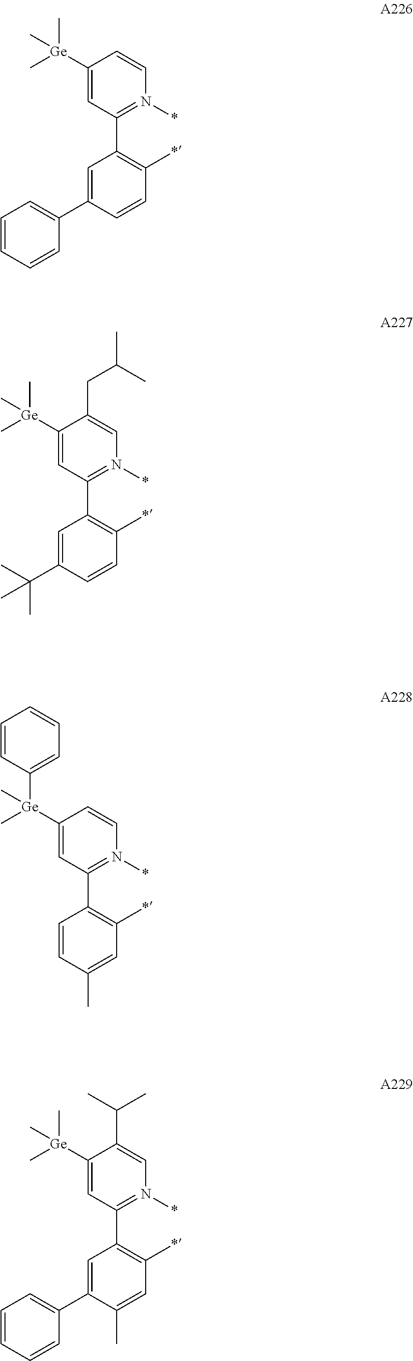

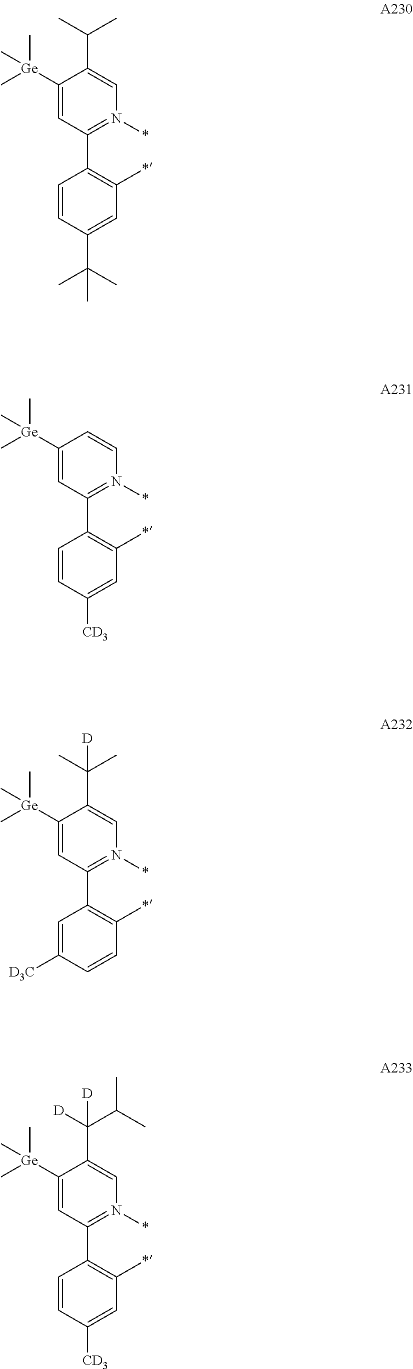

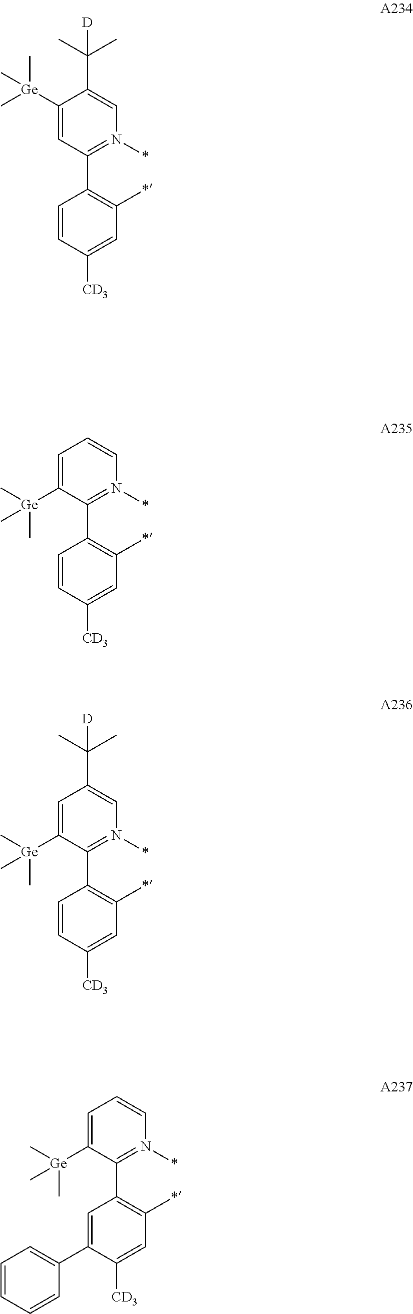

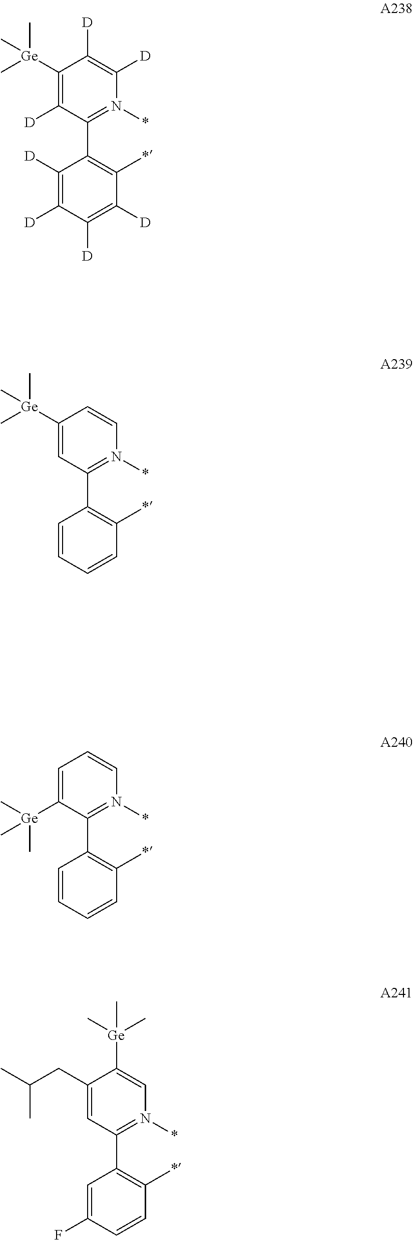

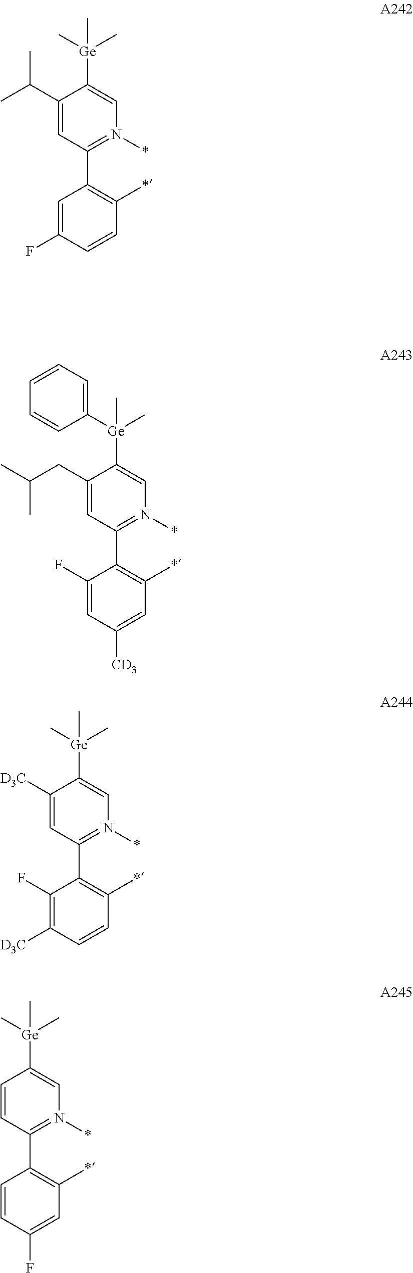

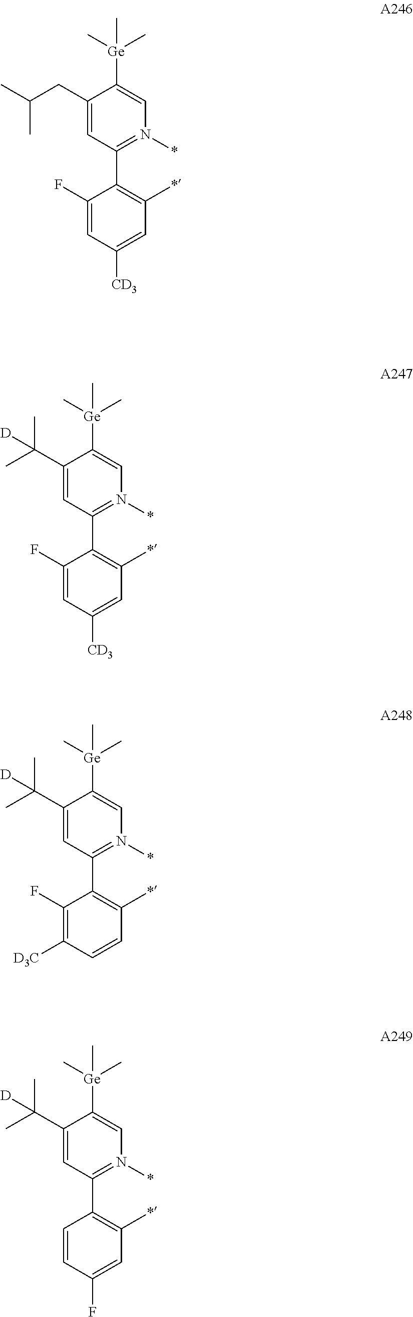

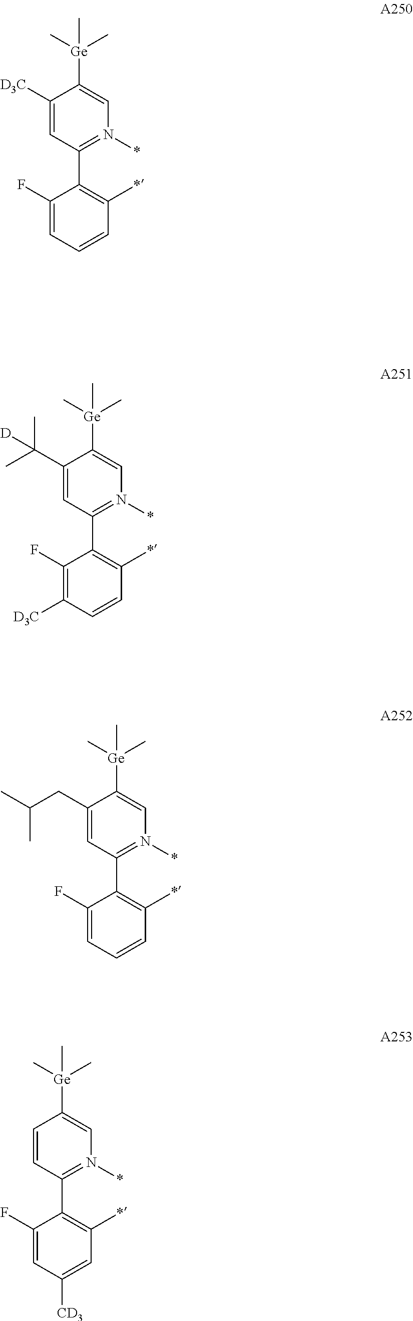

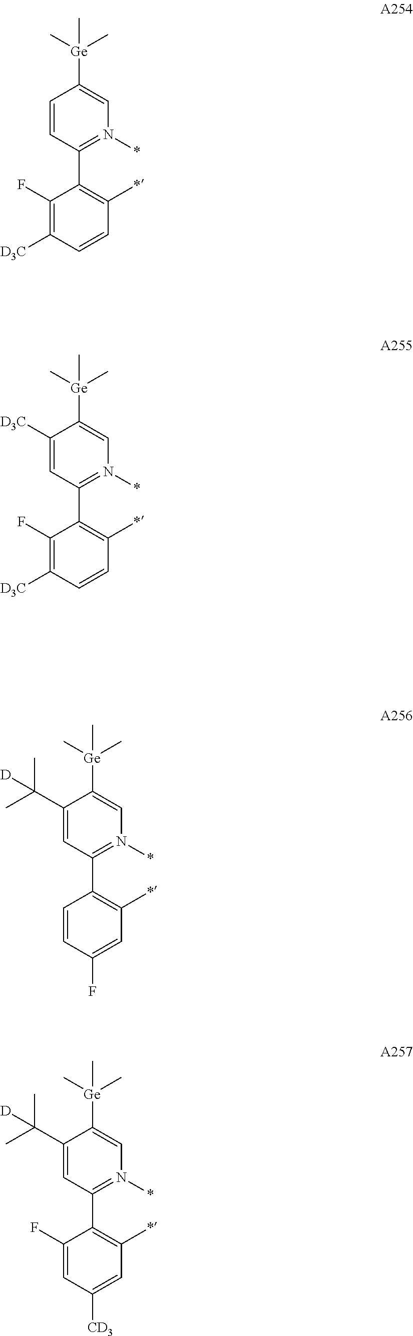

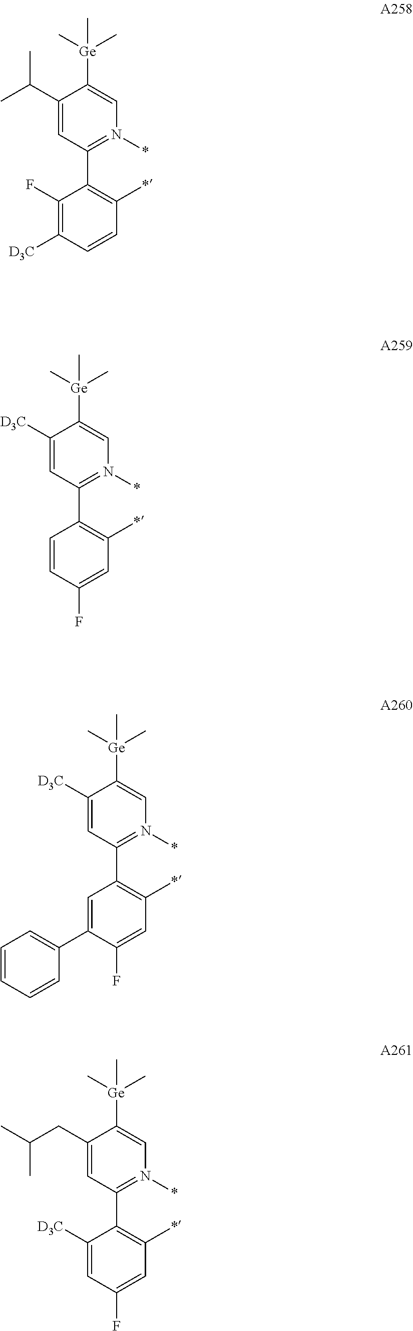

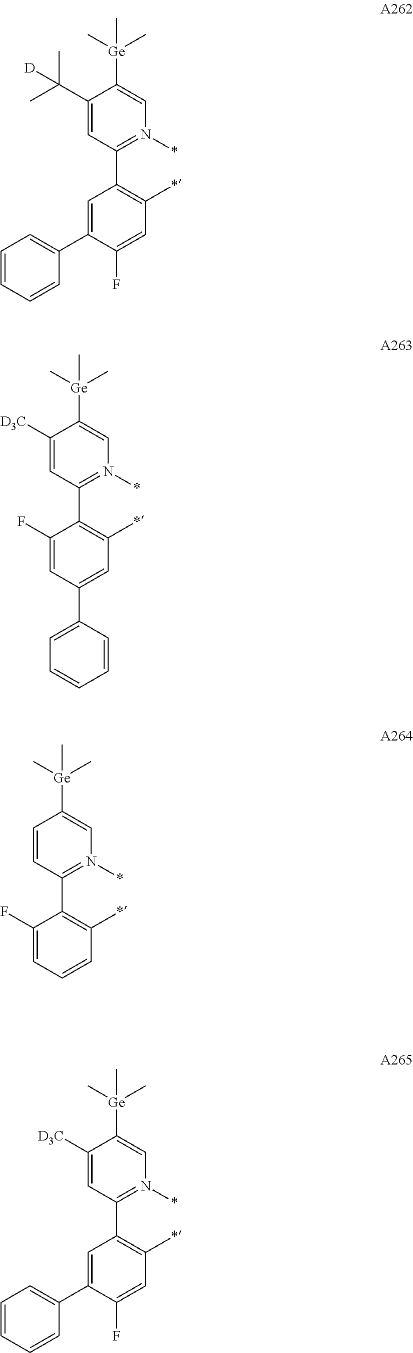

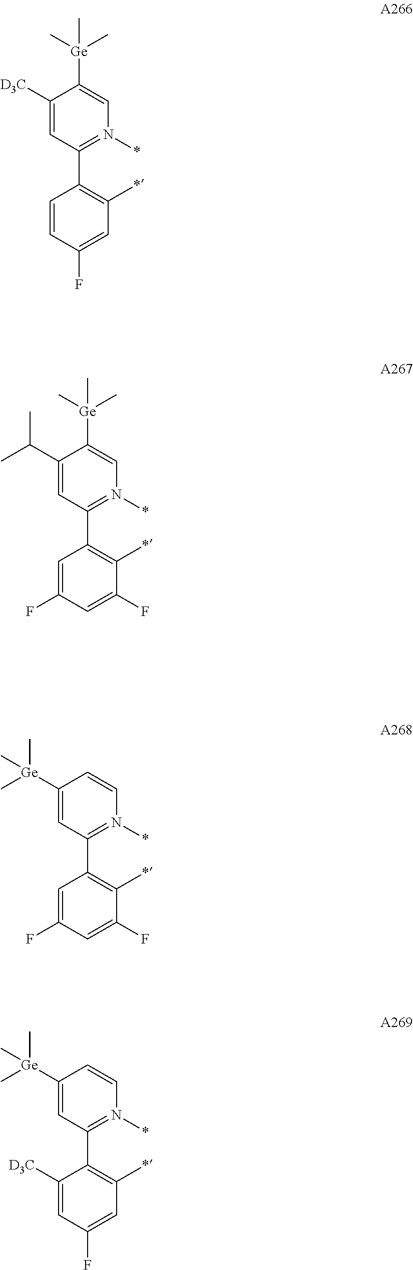

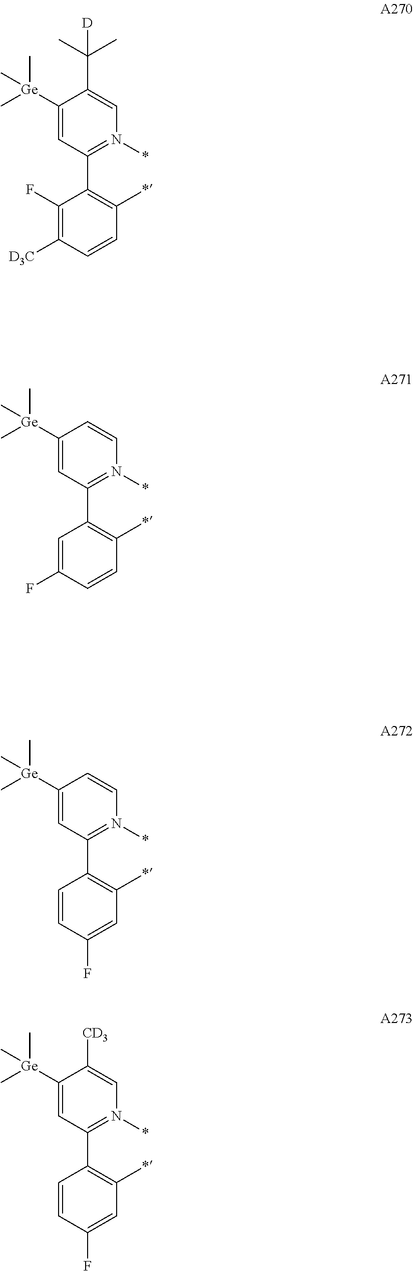

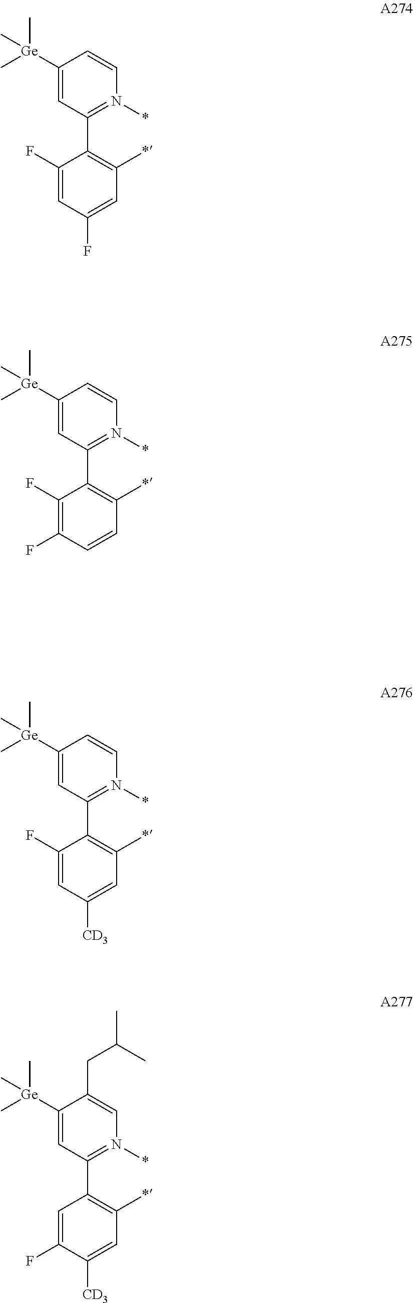

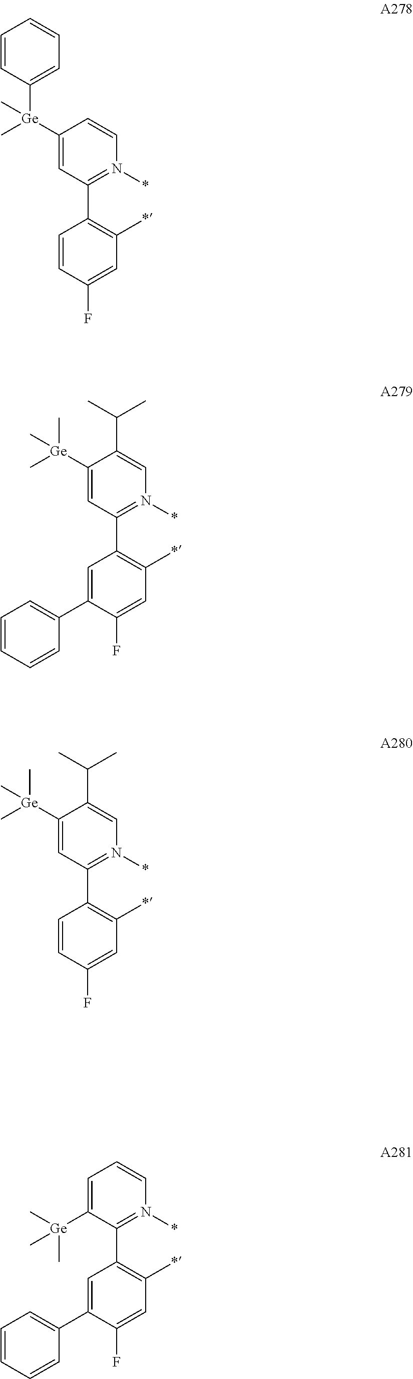

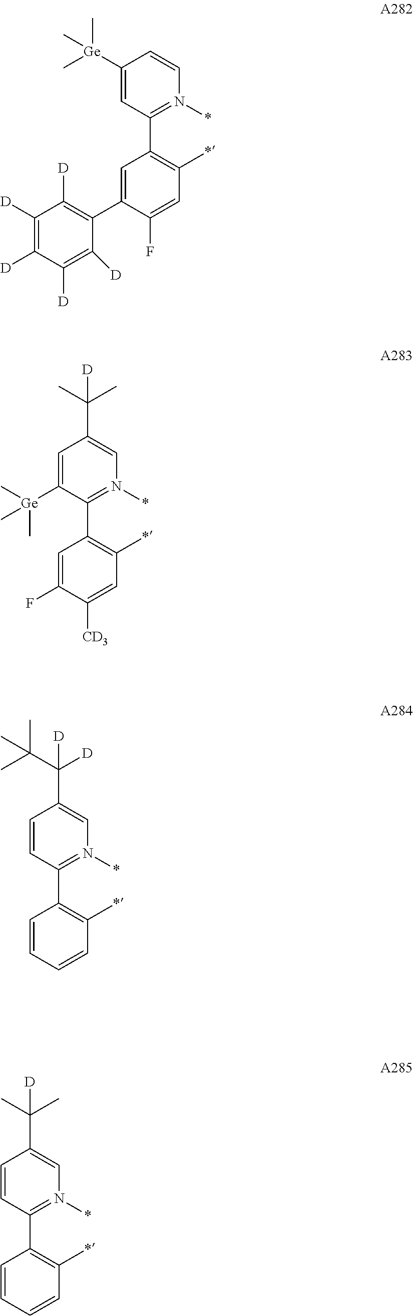

* and *′ in Formulae A1 to A285 each indicate a binding site to M₁ in Formula 1.

In one or more embodiments, Ln₂ in Formula 1 may be a ligand represented by one of Formulae B1 to B420:

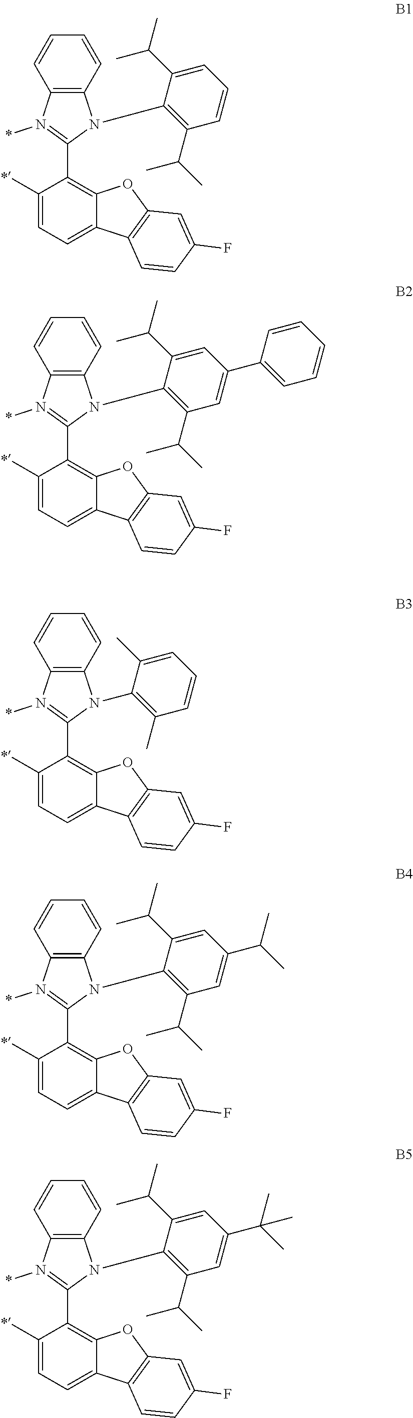

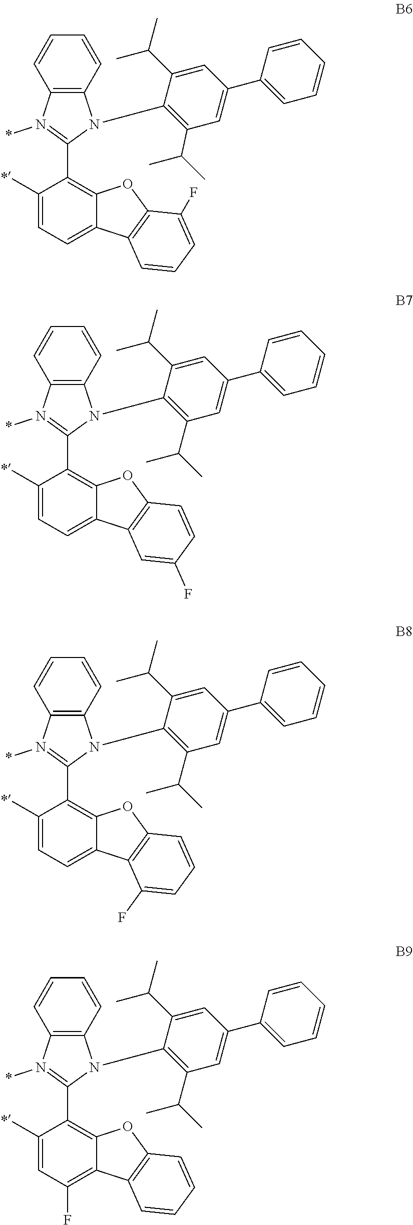

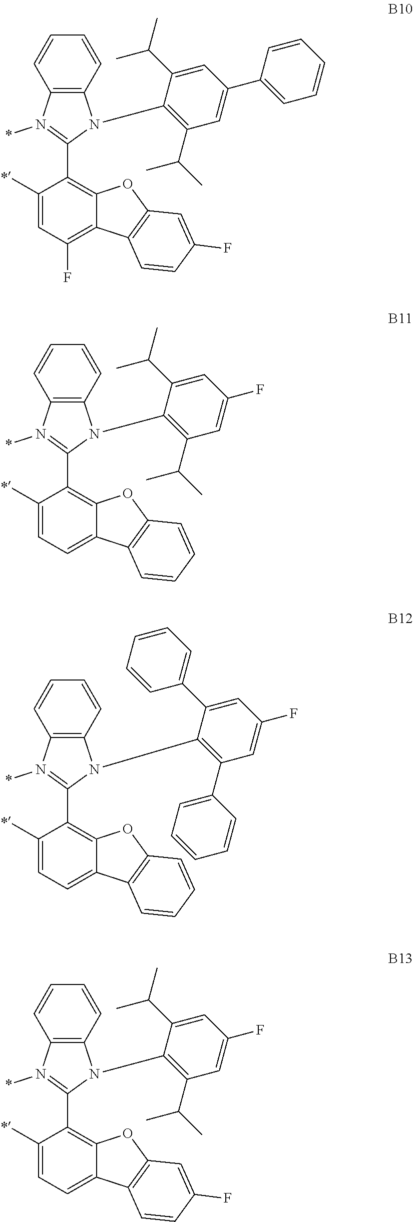

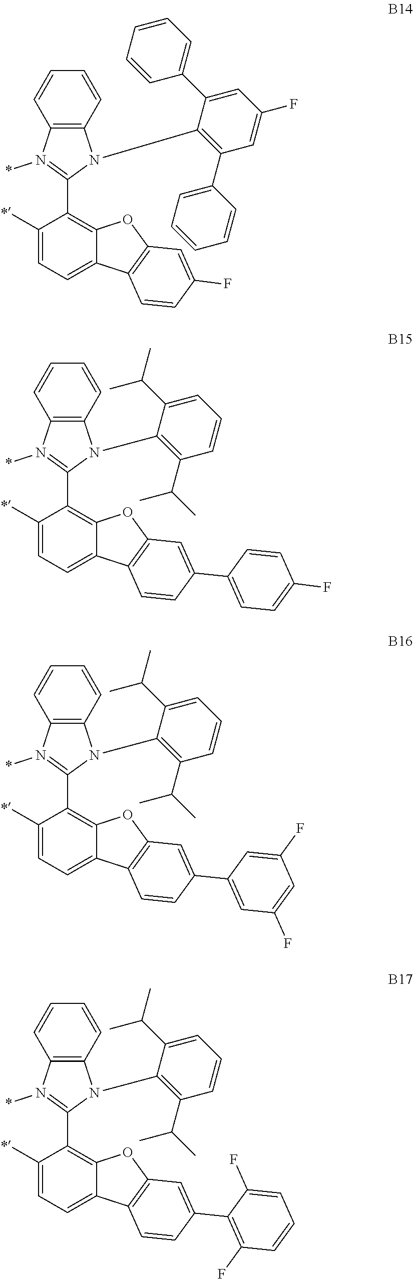

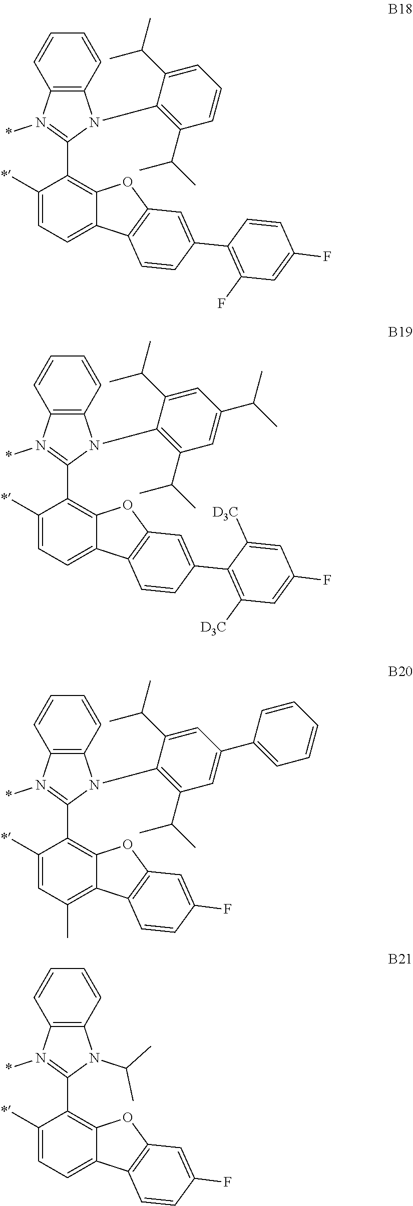

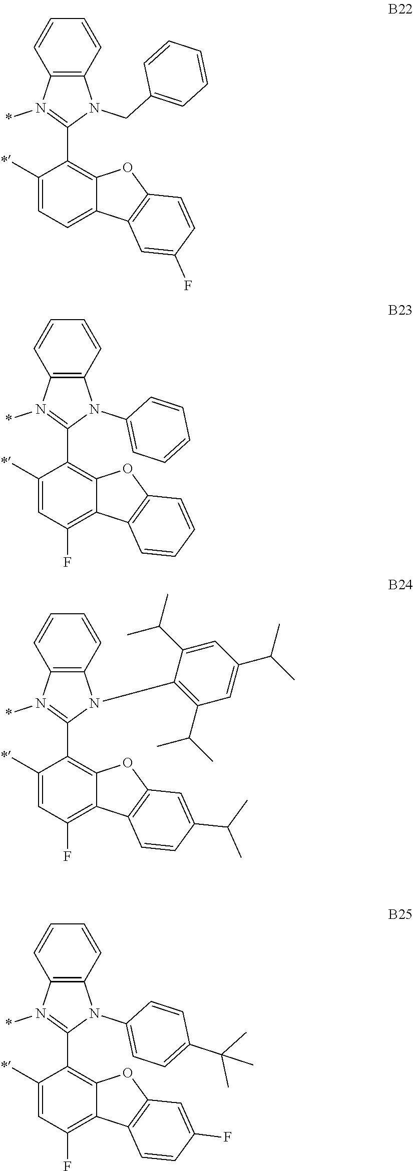

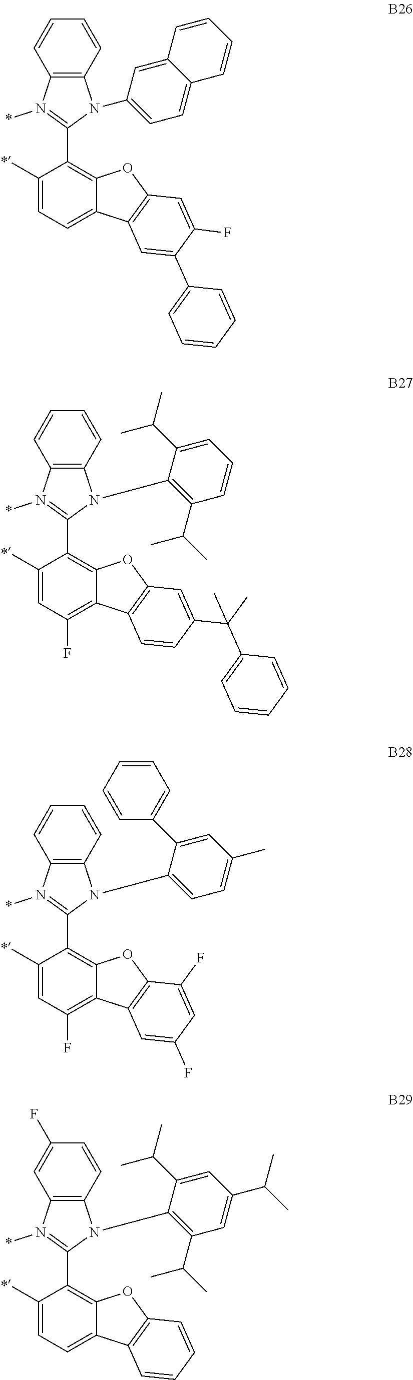

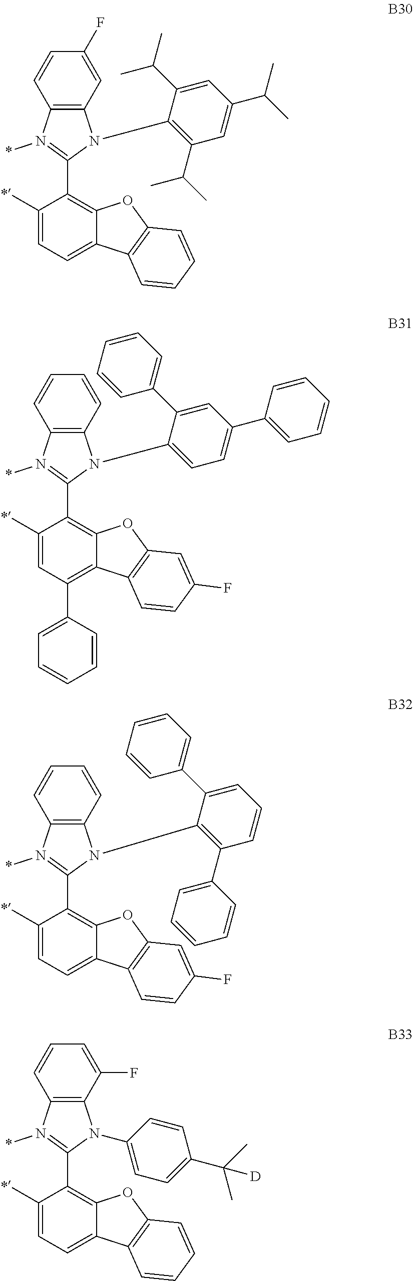

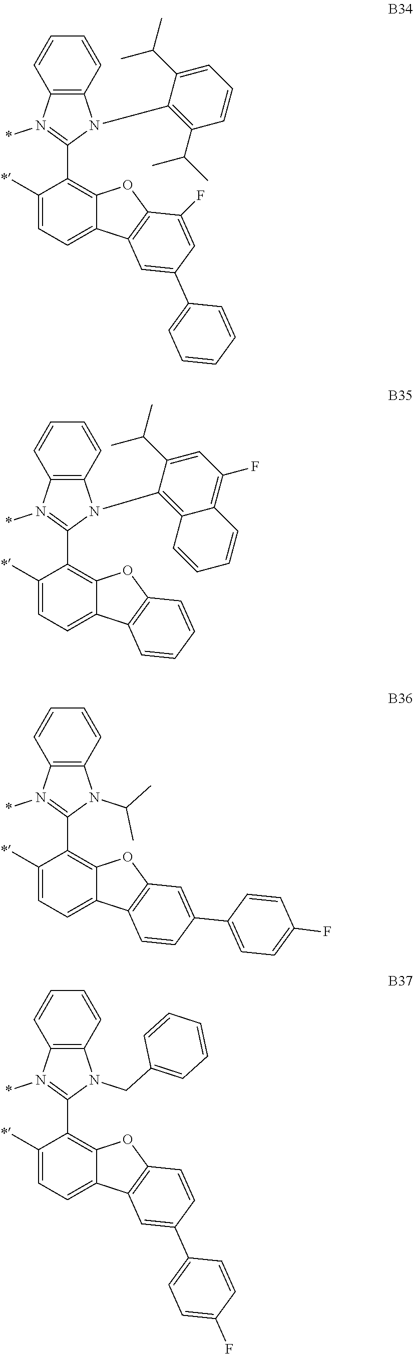

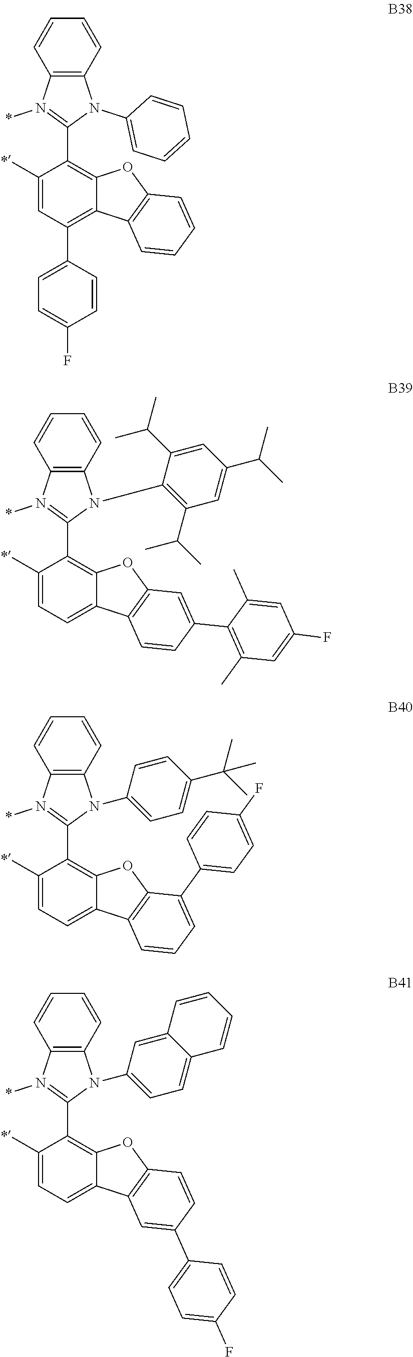

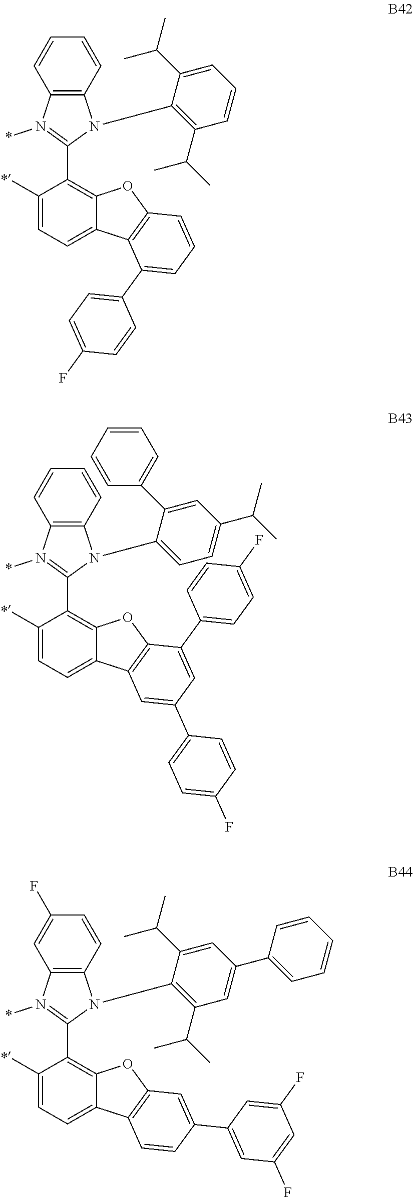

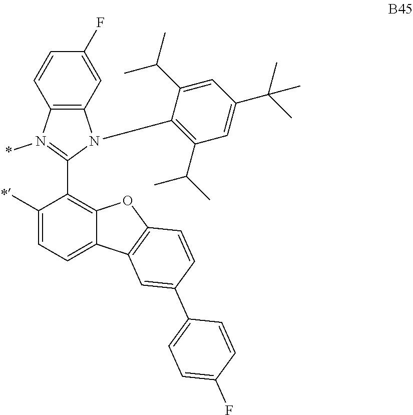

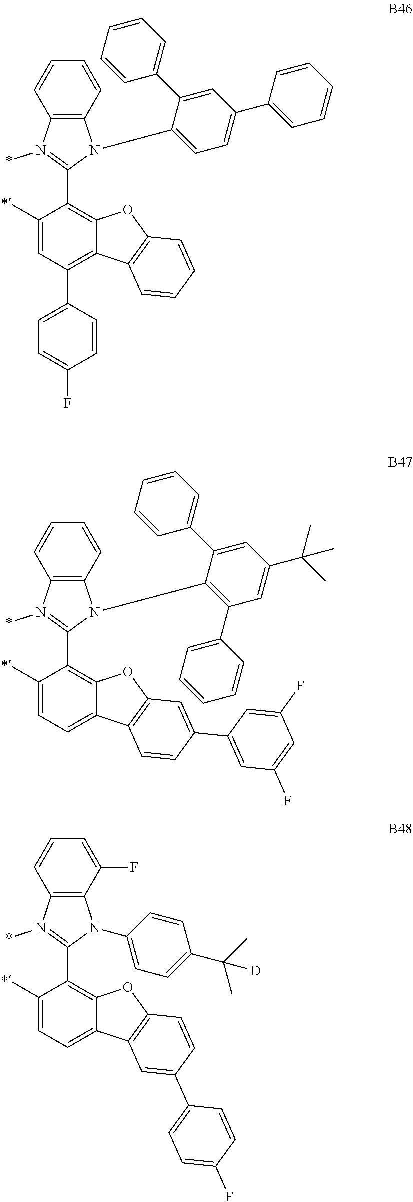

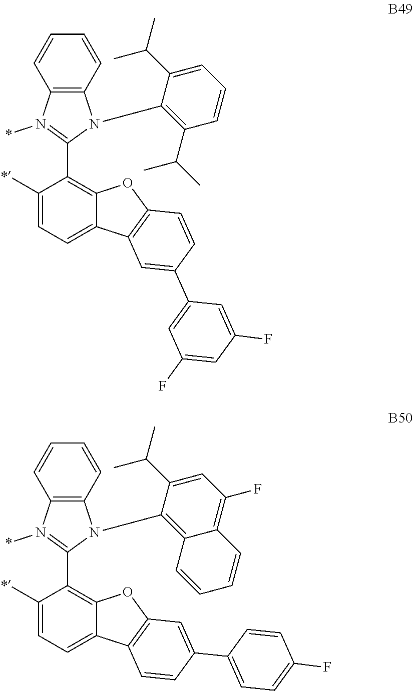

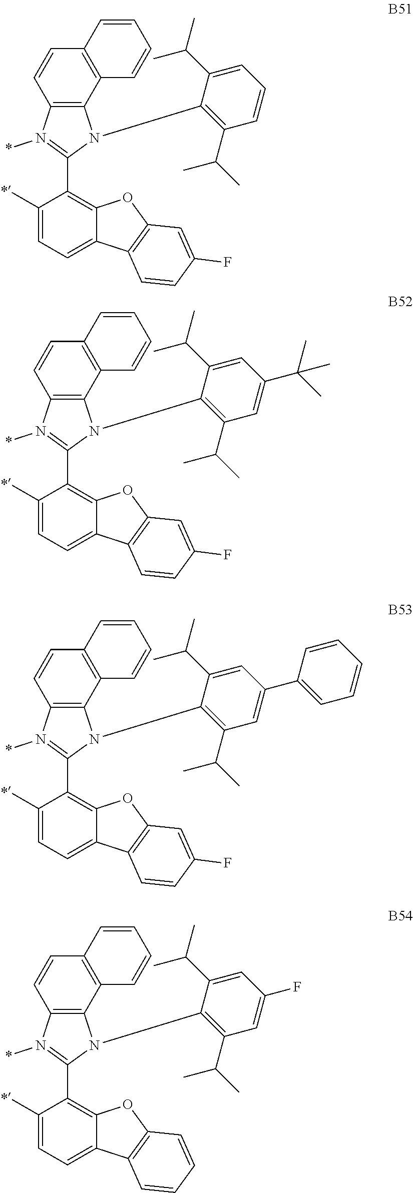

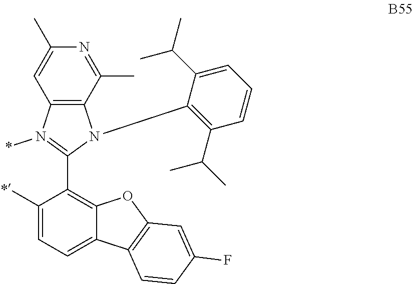

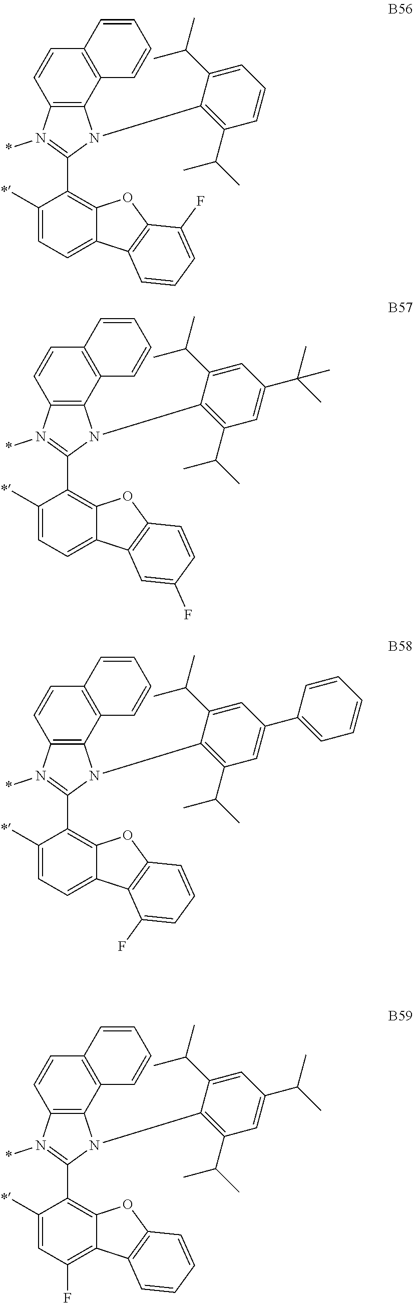

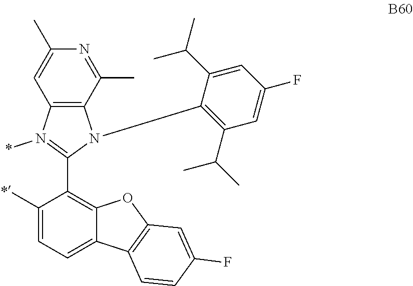

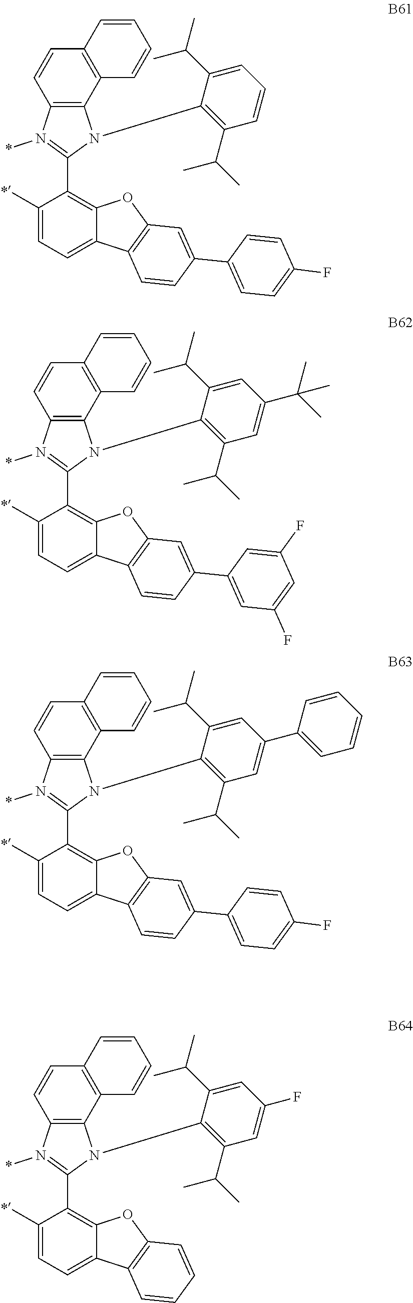

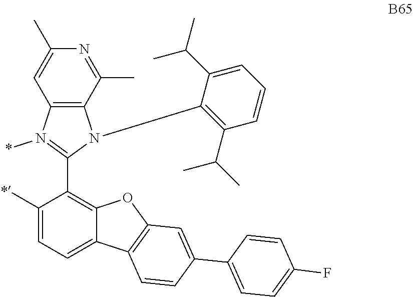

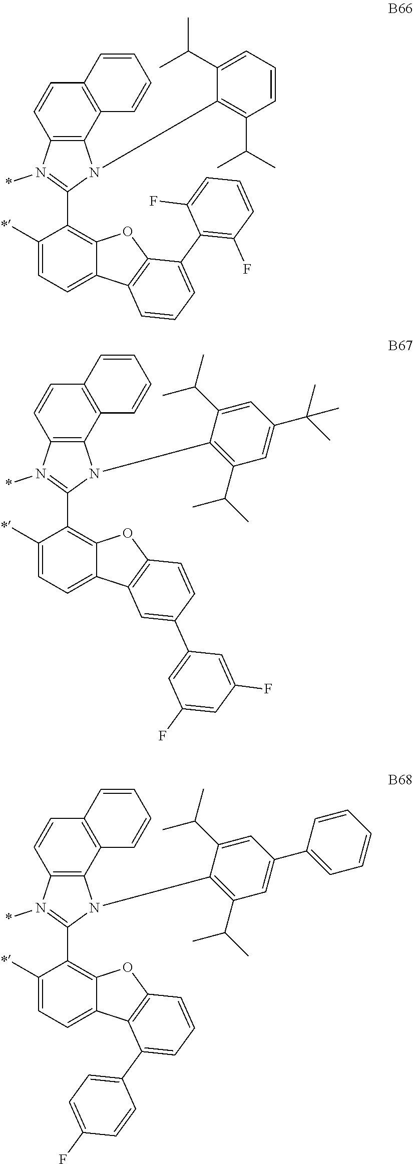

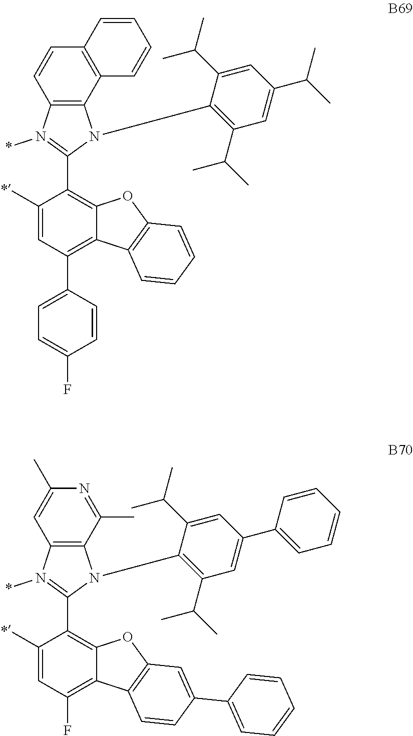

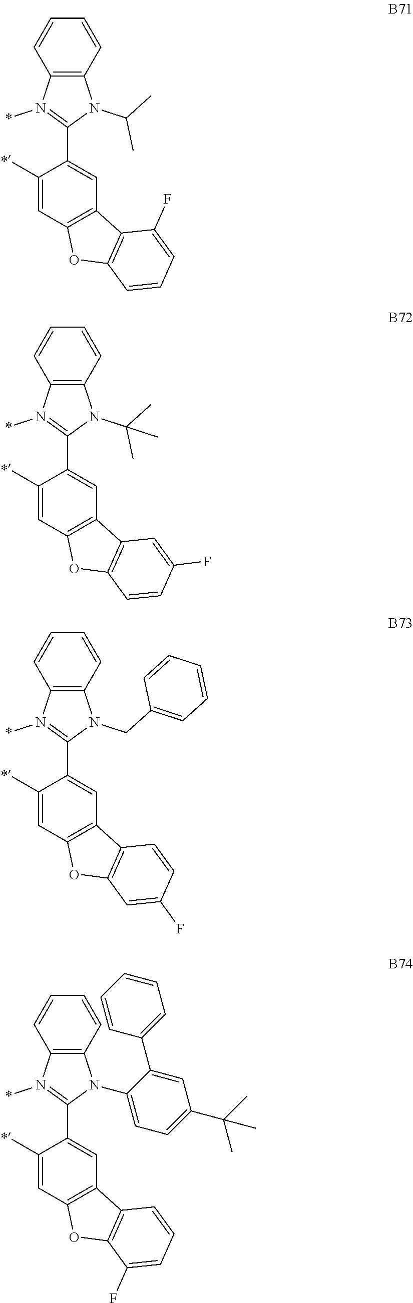

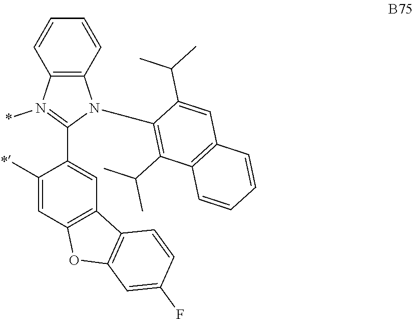

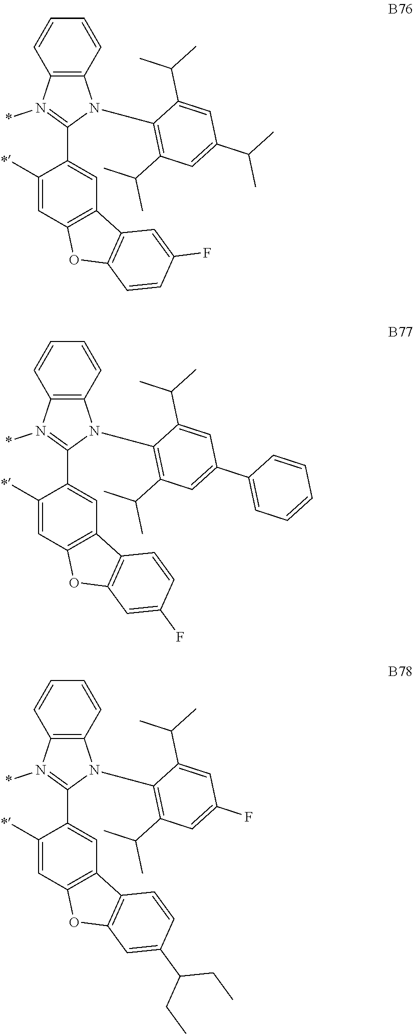

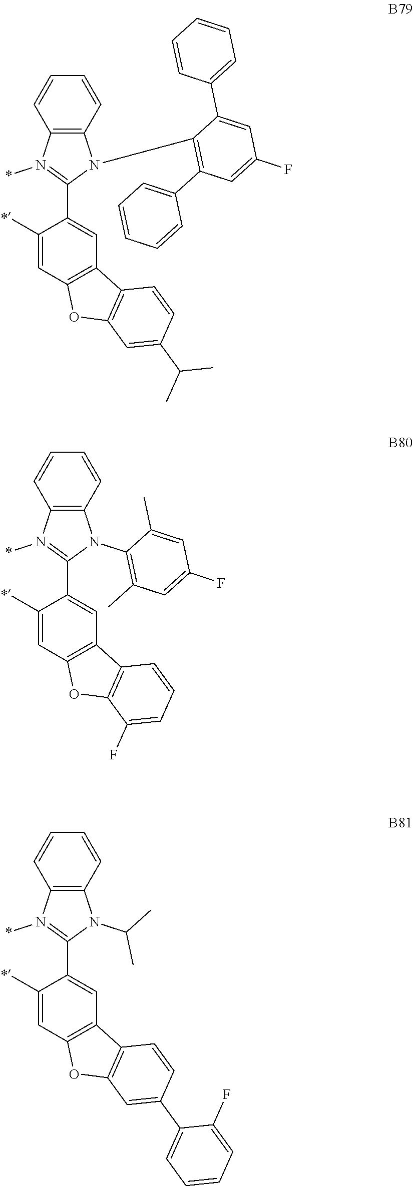

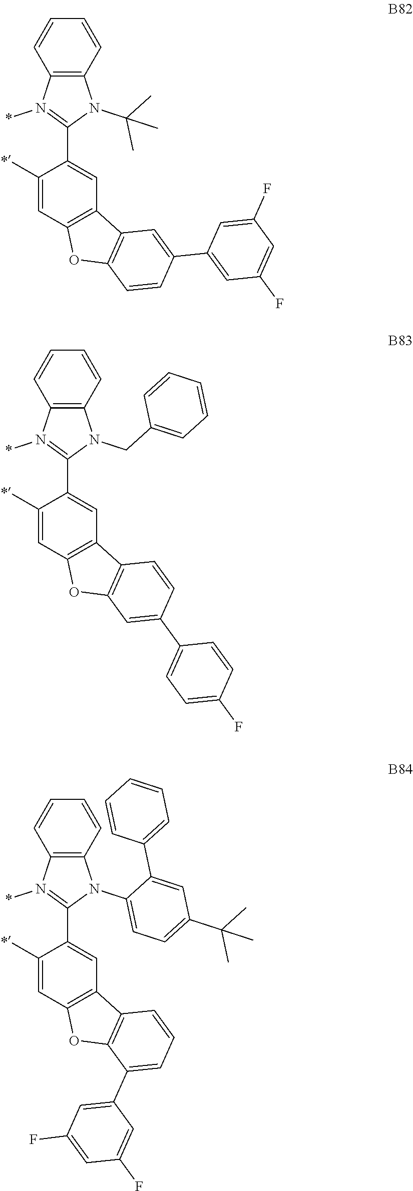

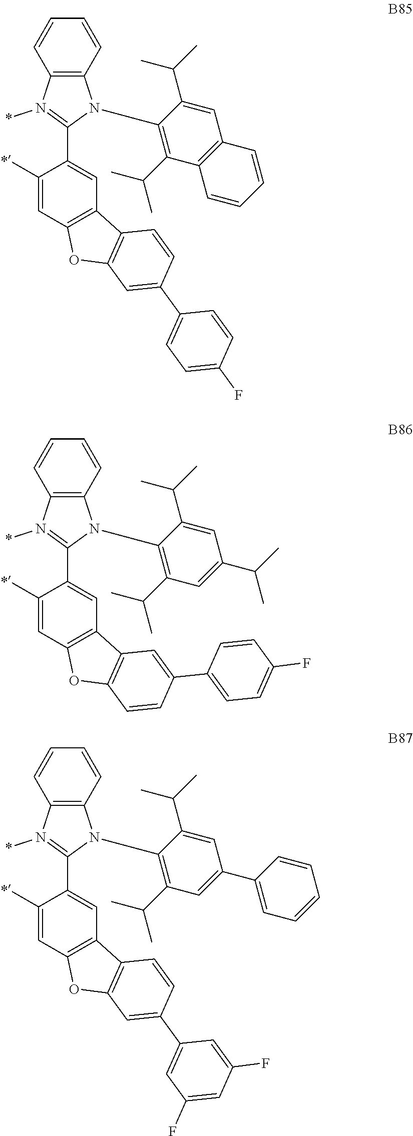

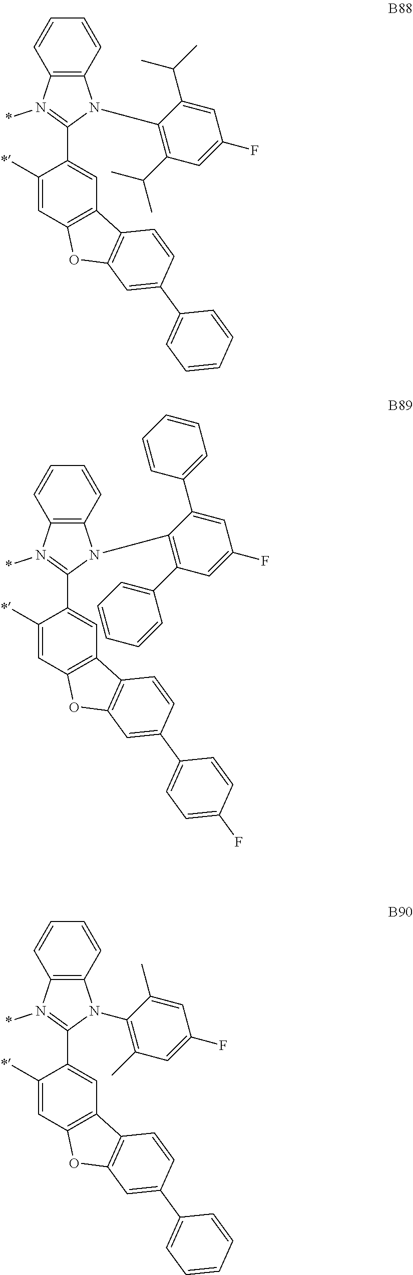

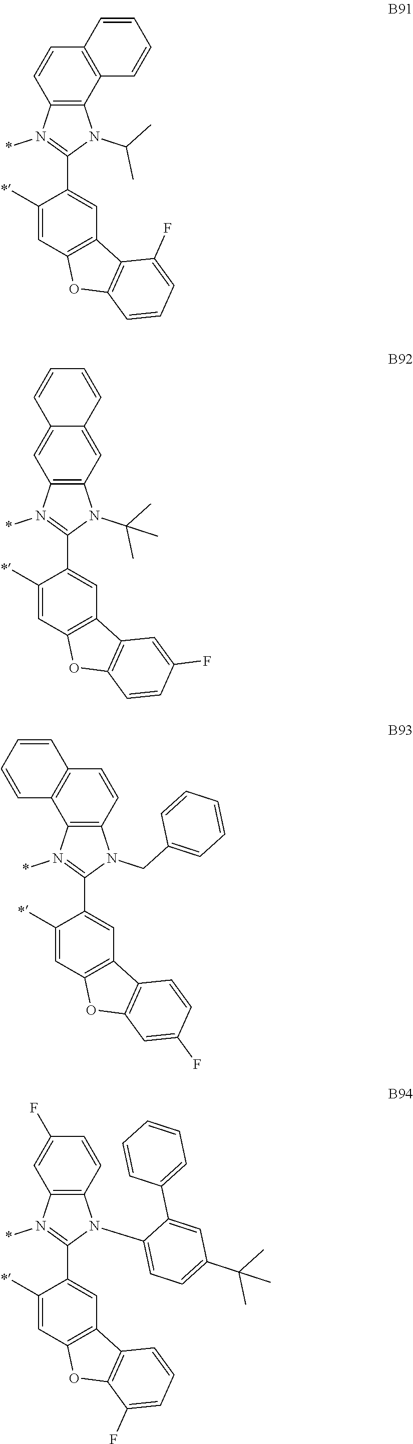

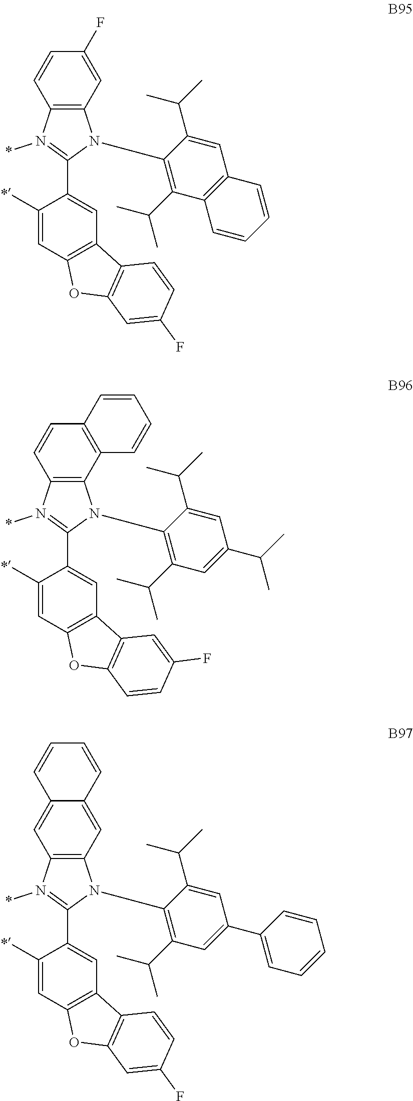

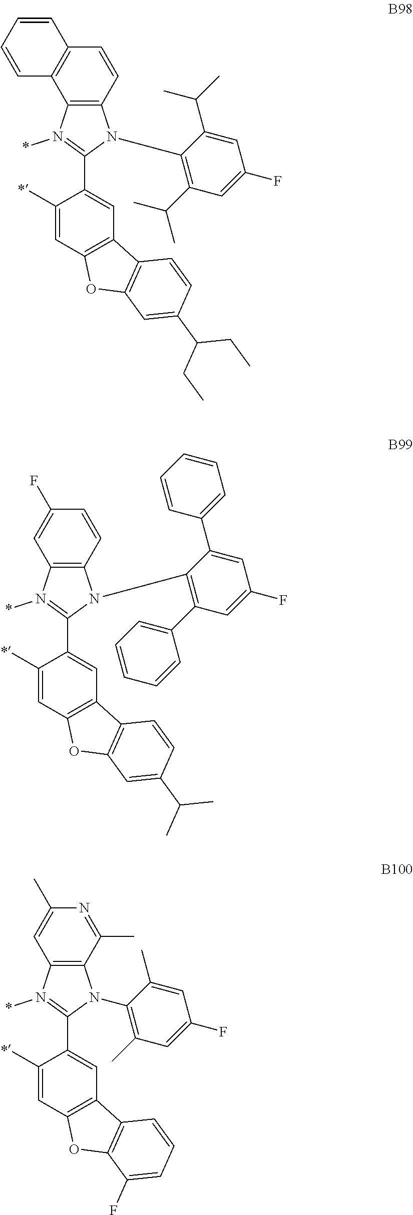

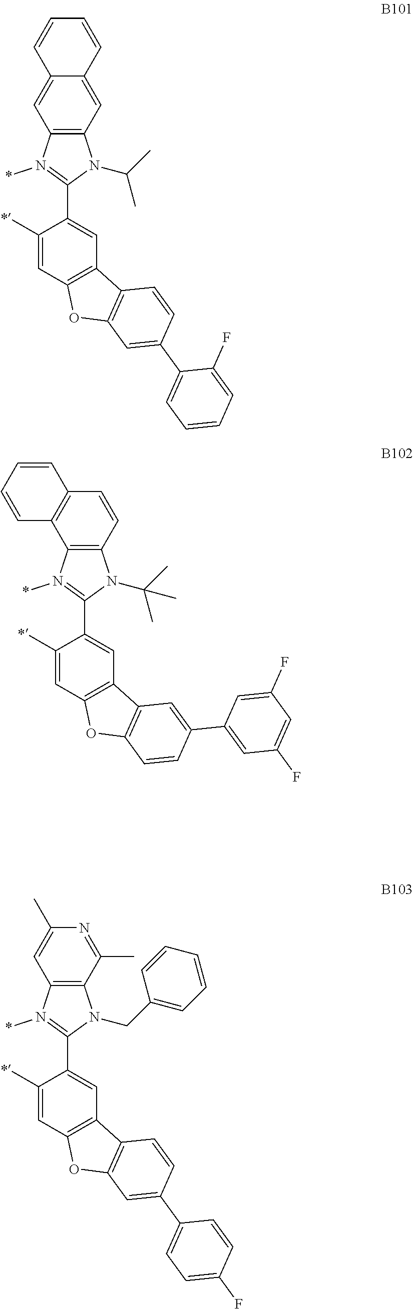

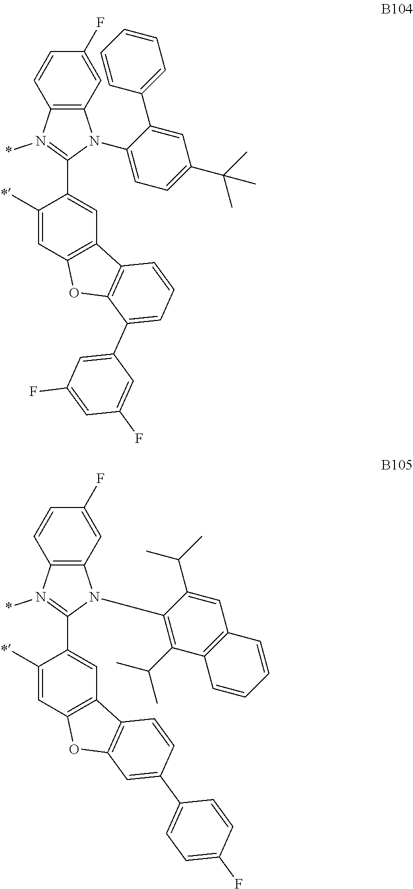

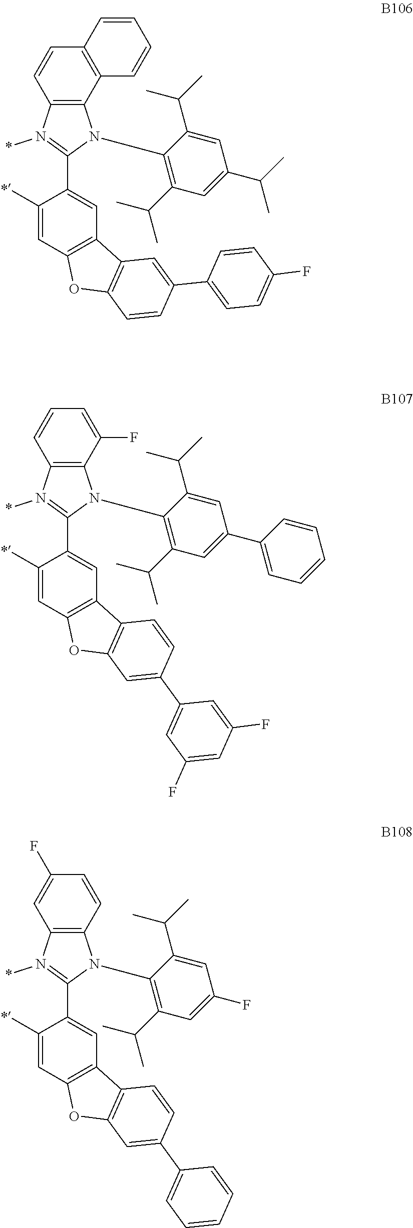

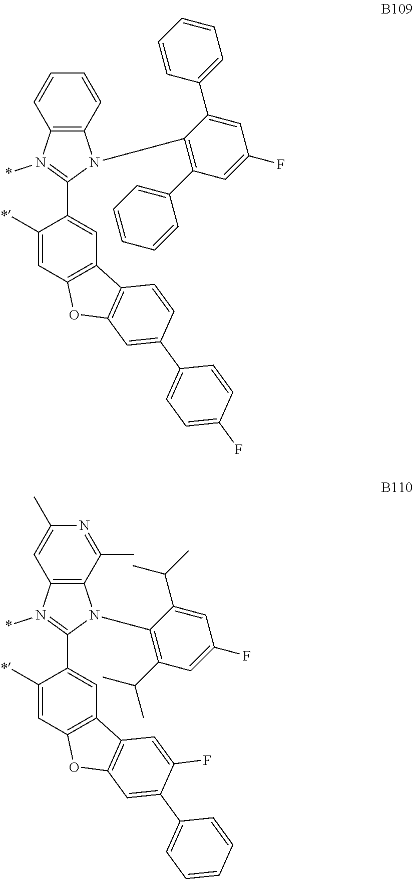

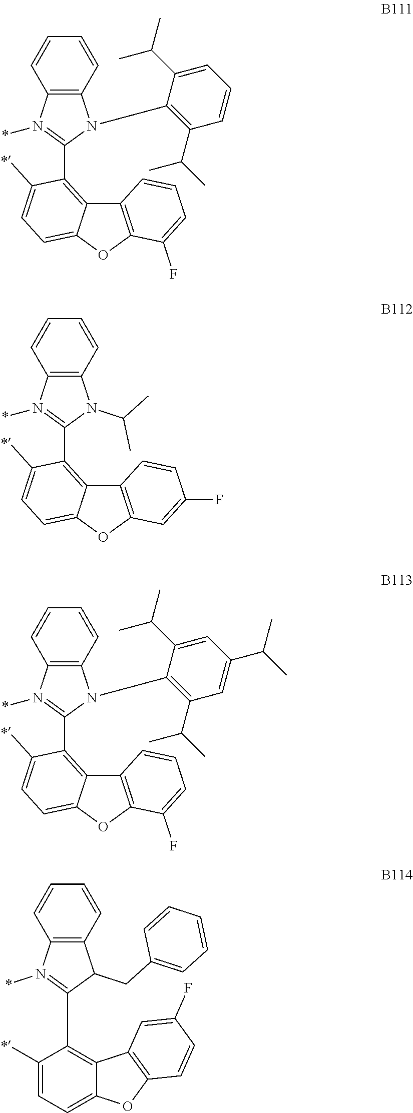

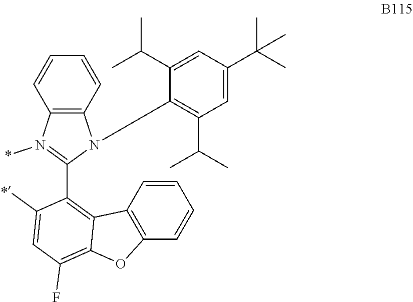

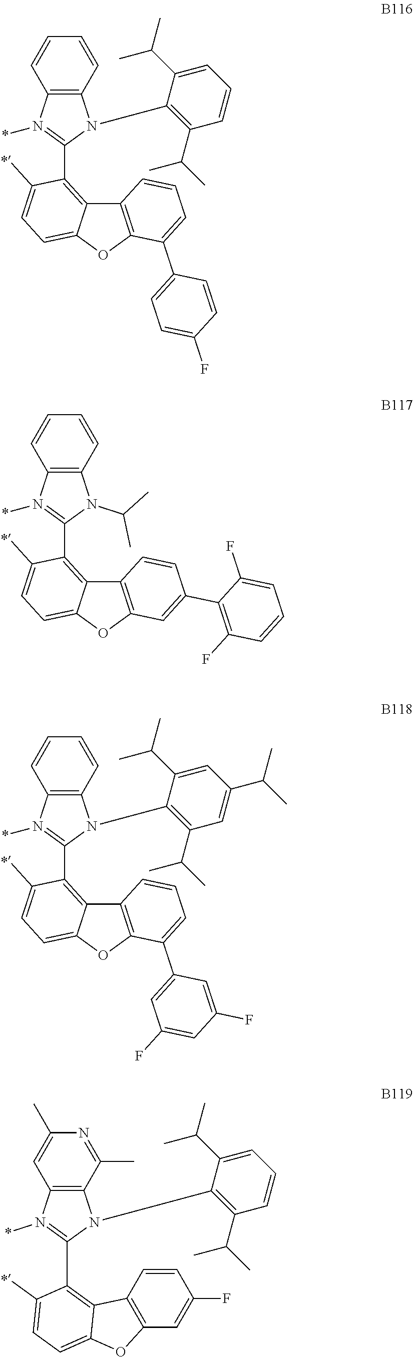

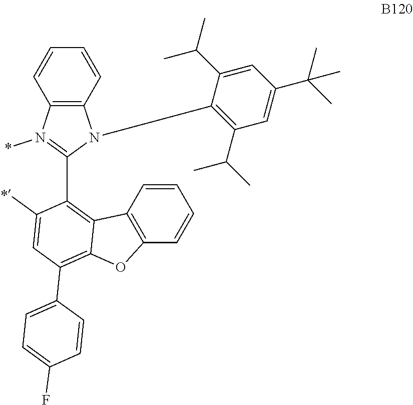

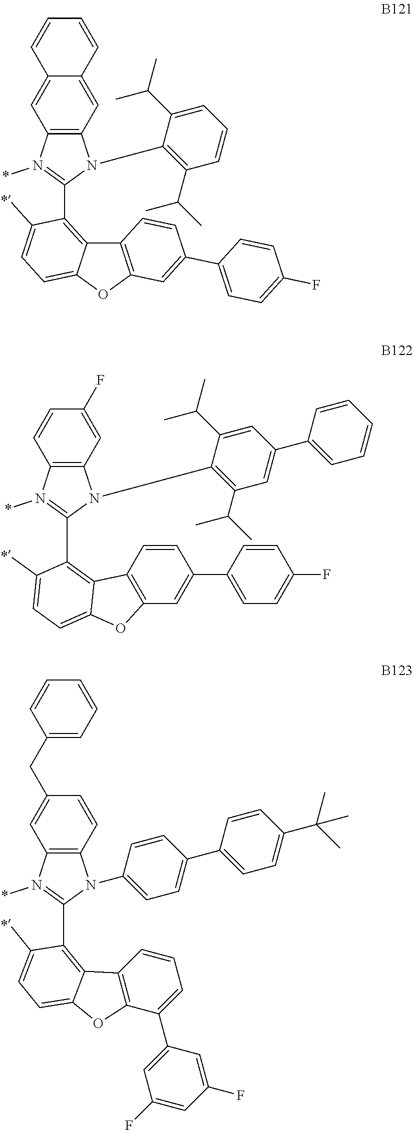

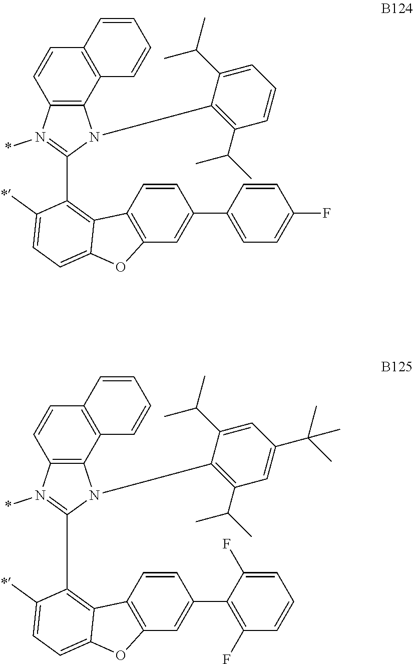

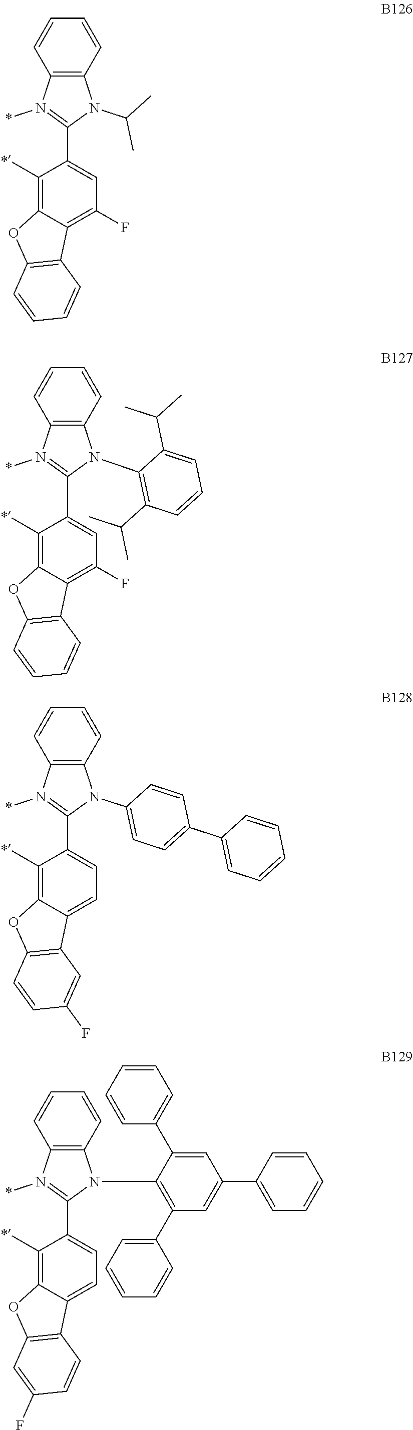

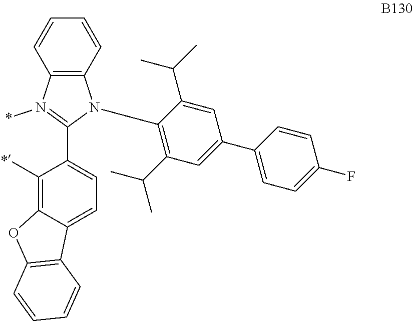

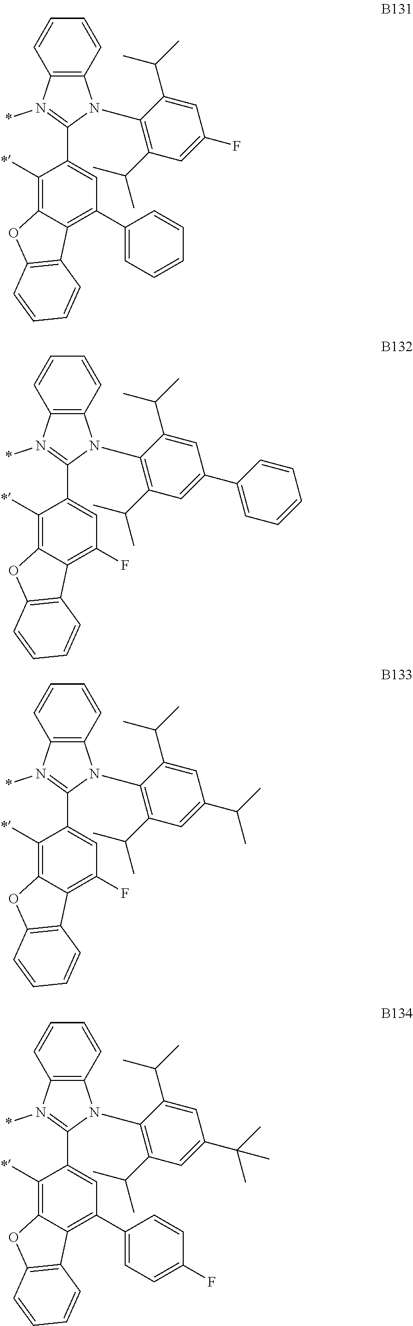

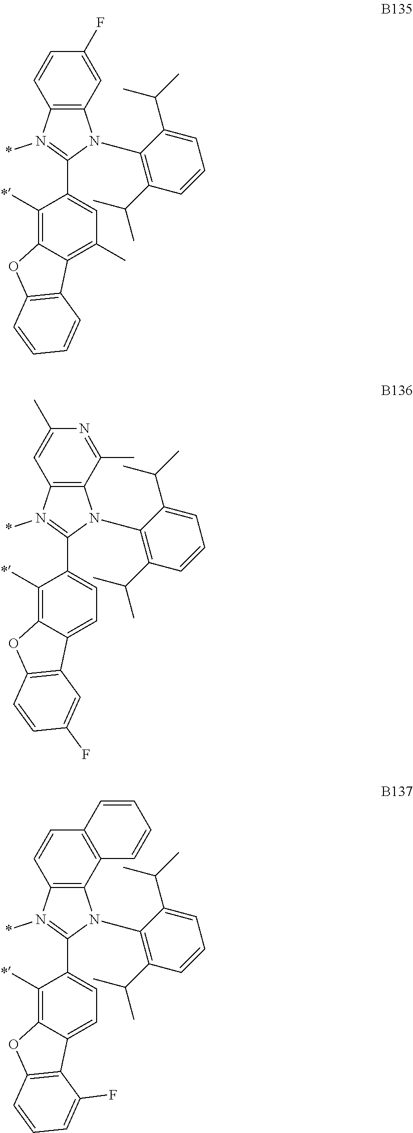

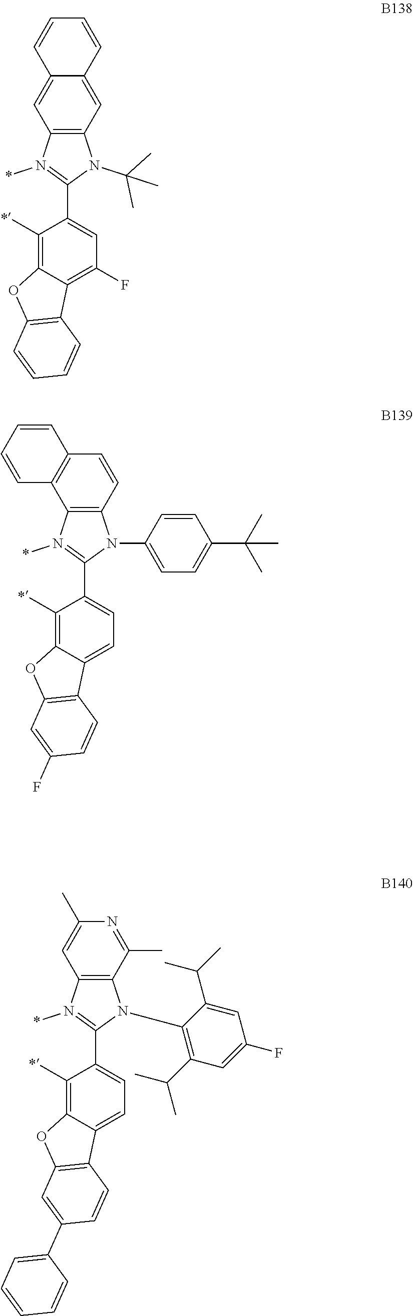

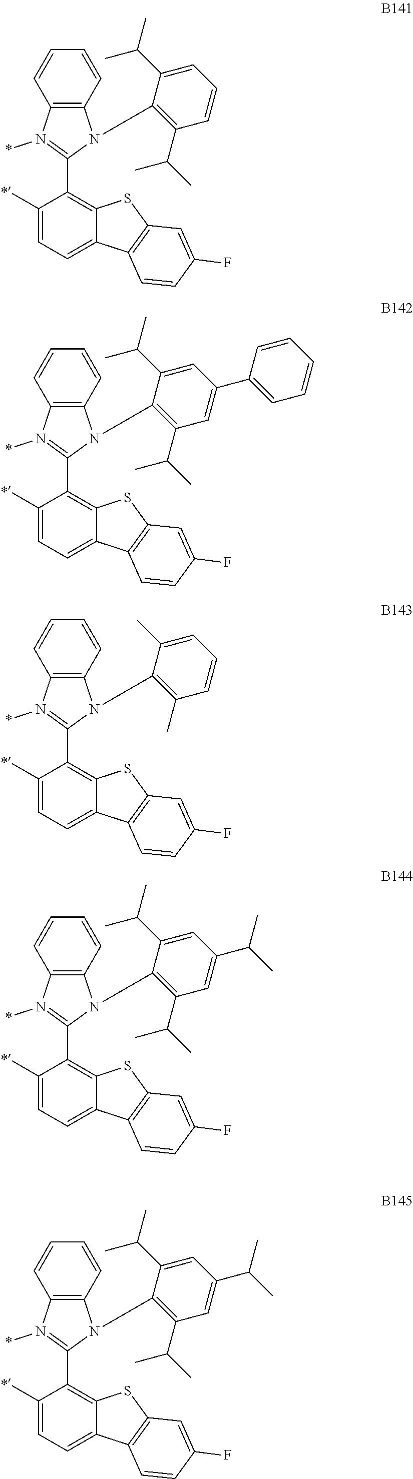

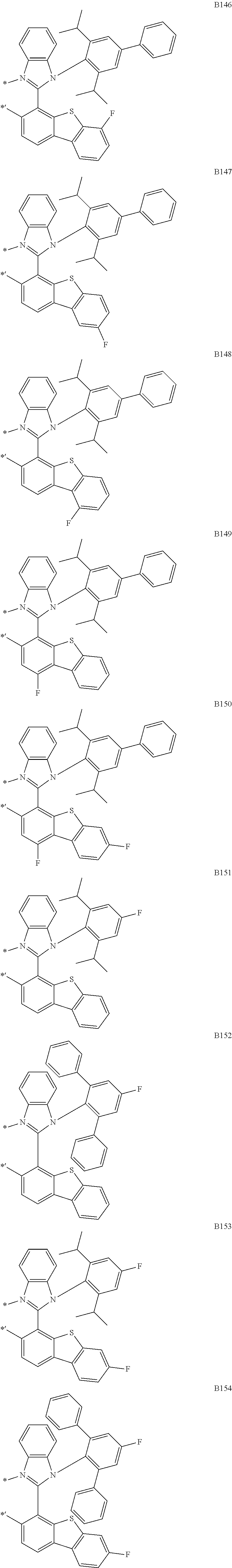

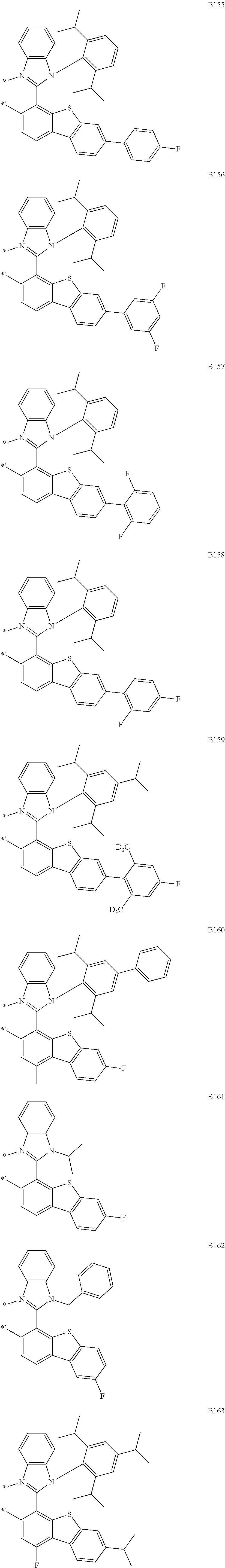

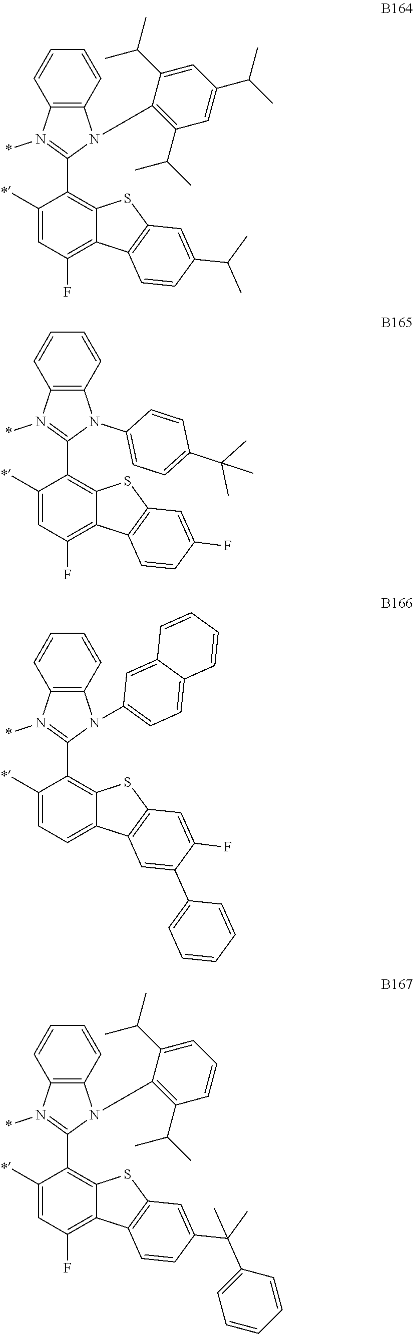

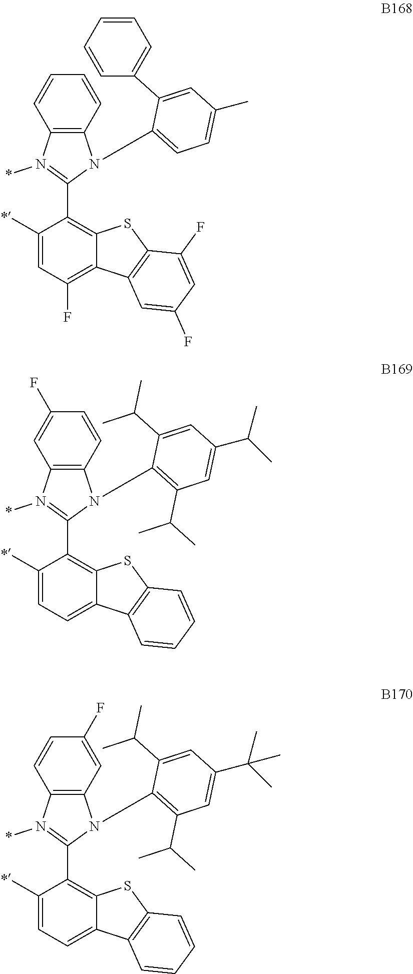

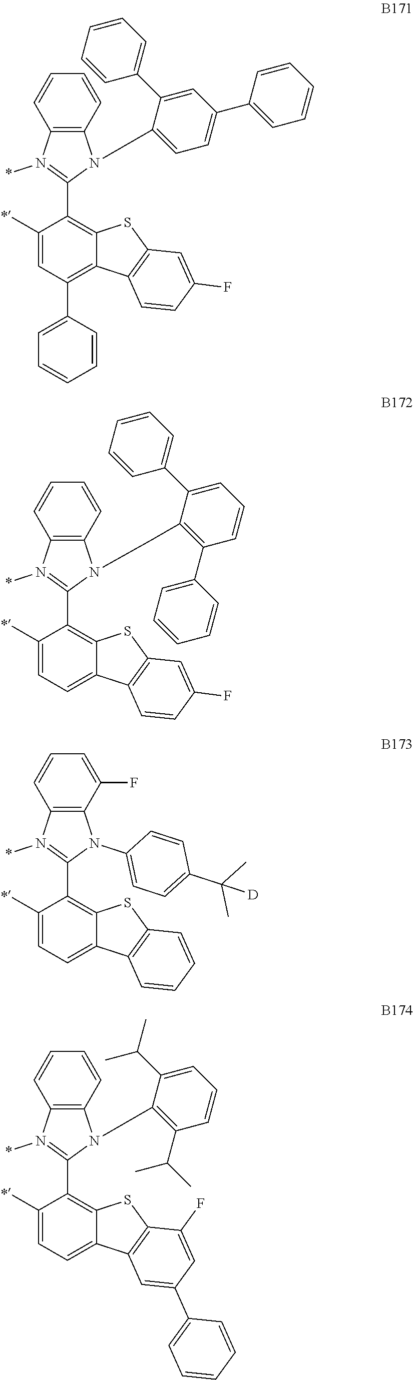

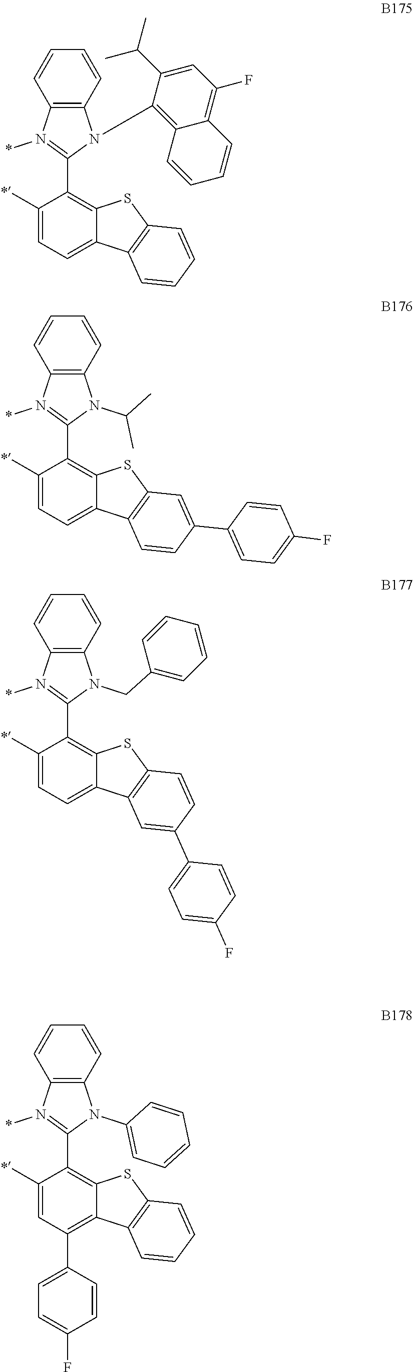

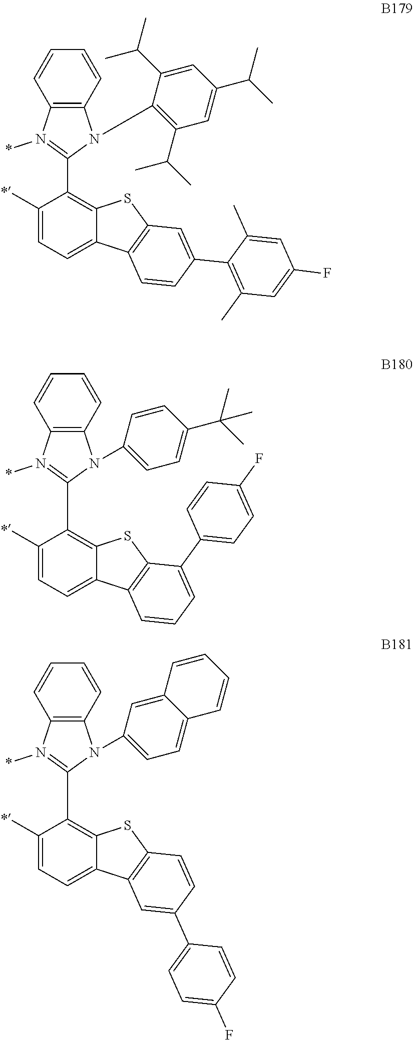

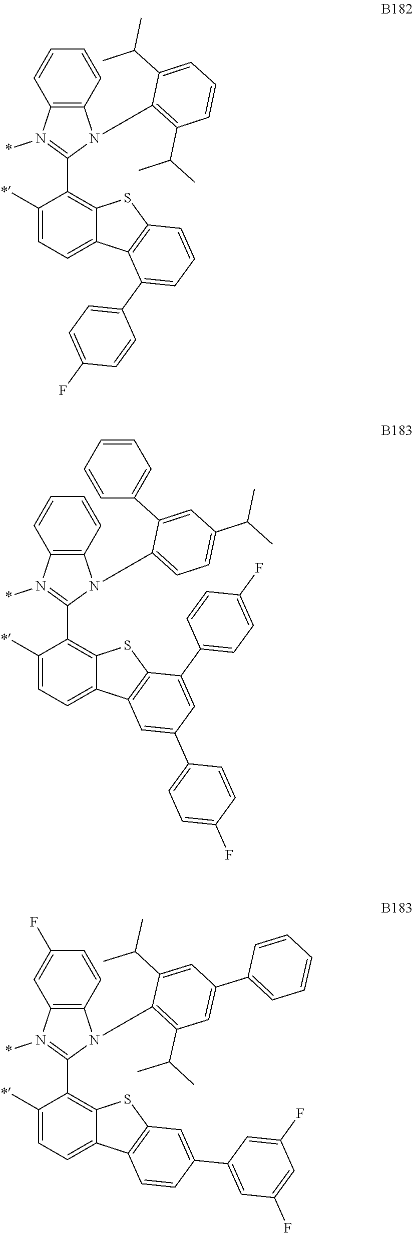

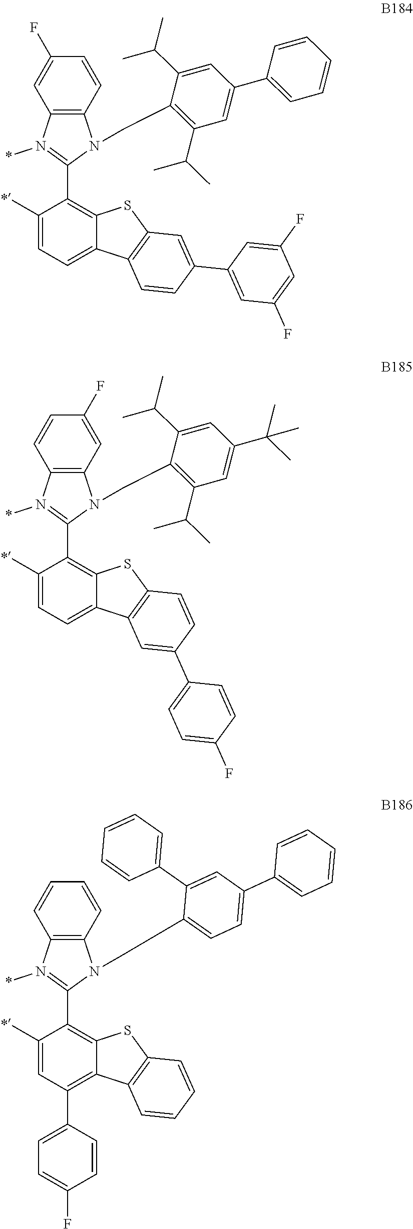

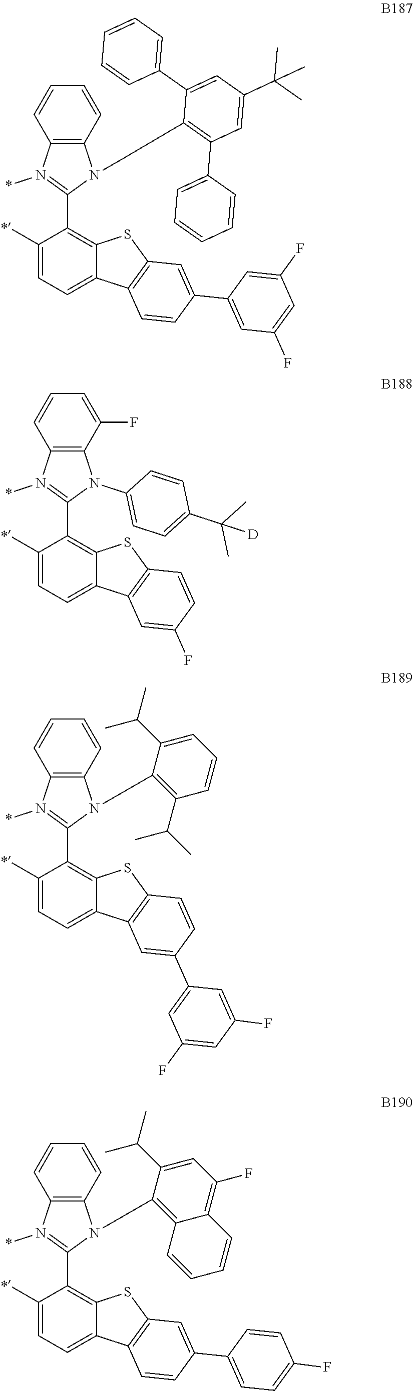

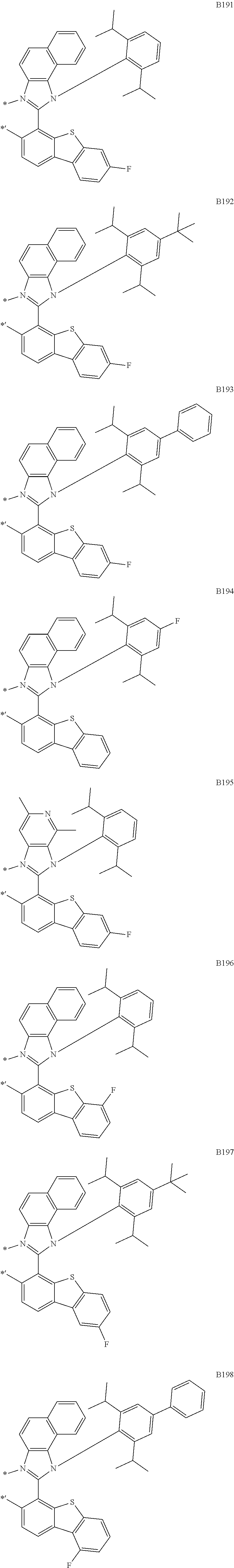

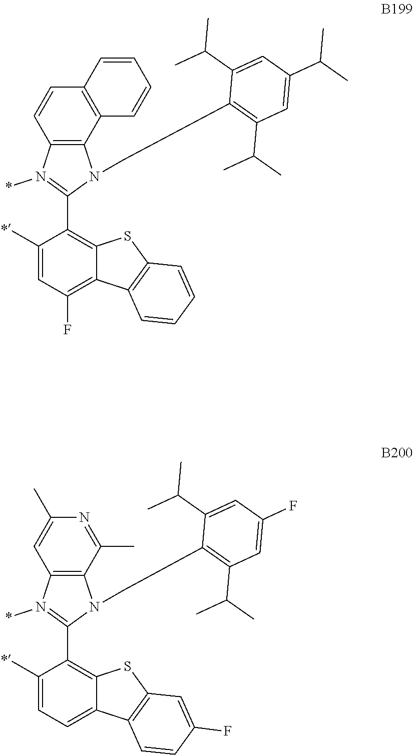

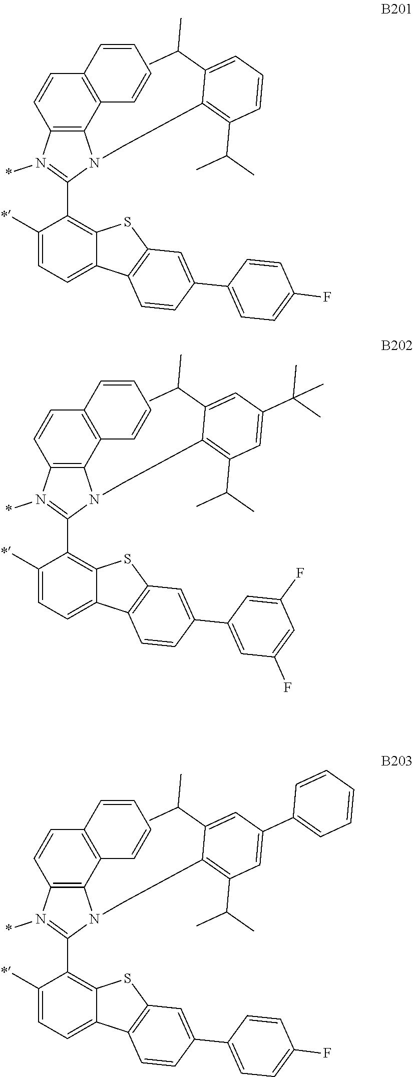

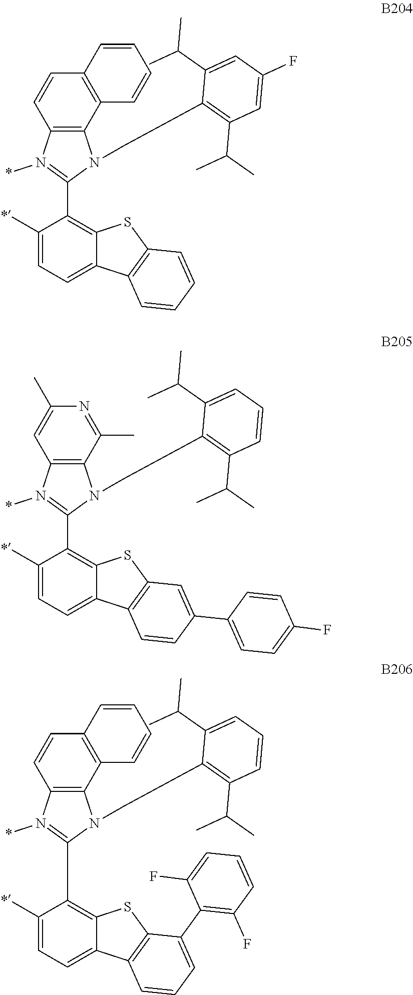

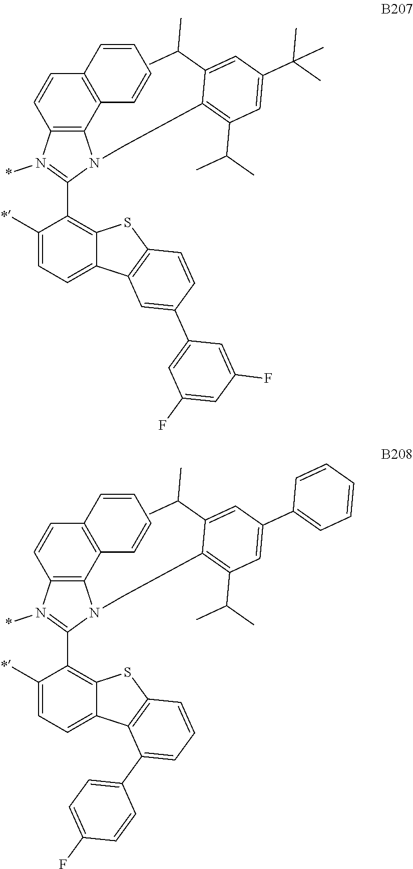

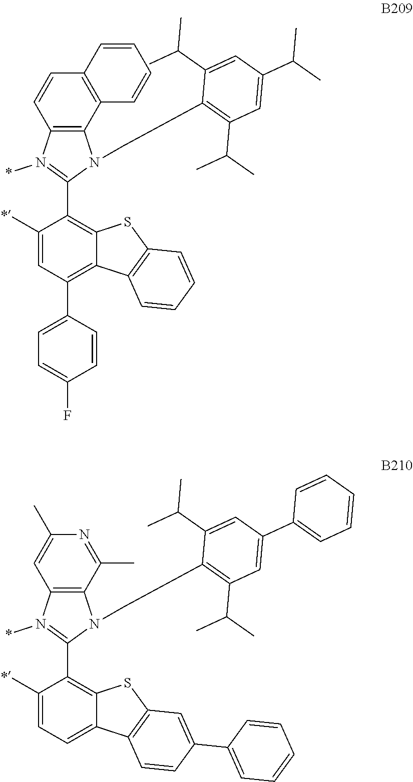

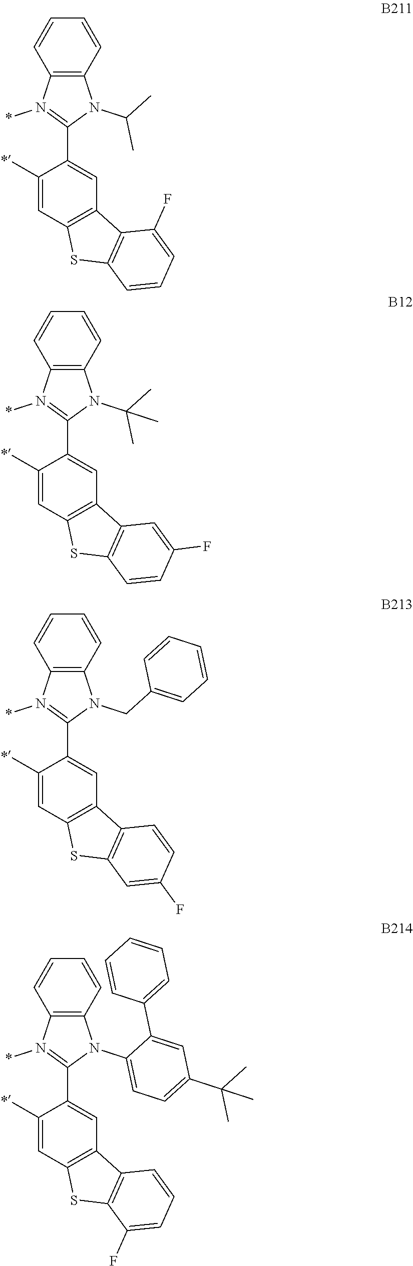

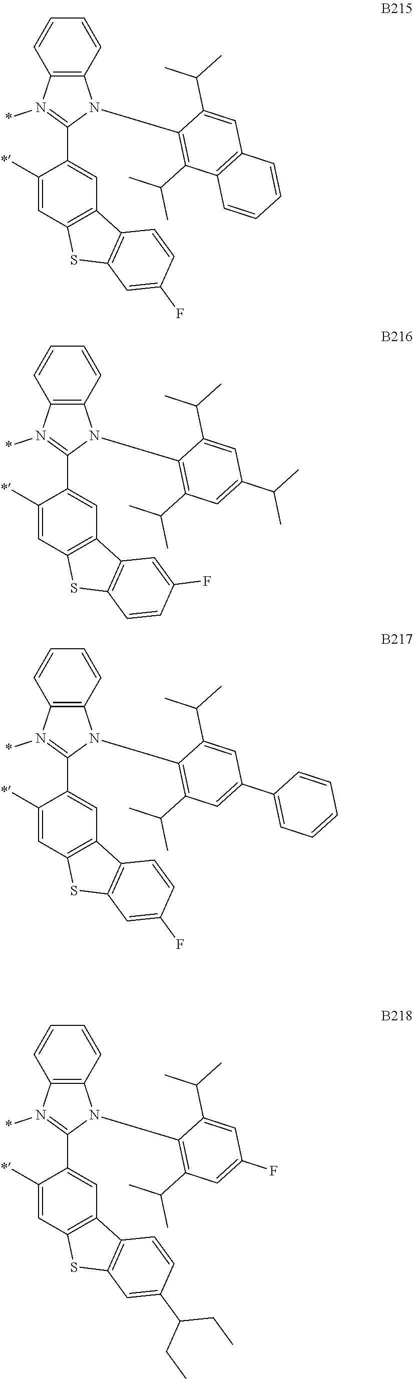

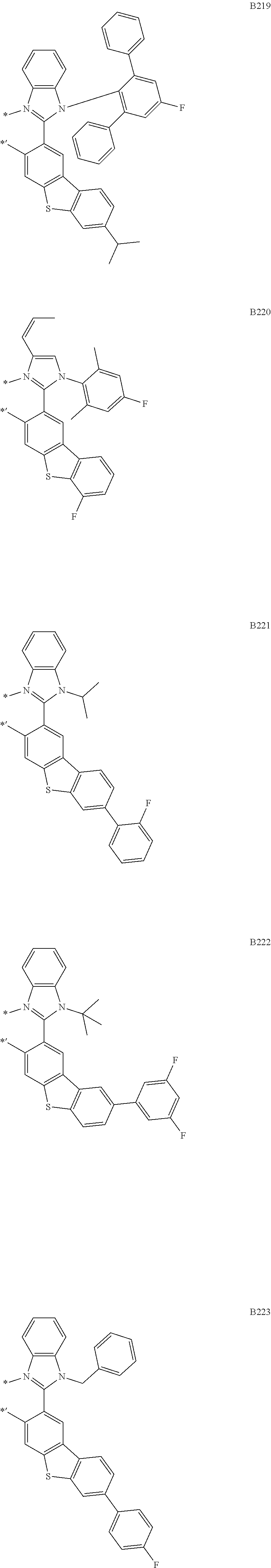

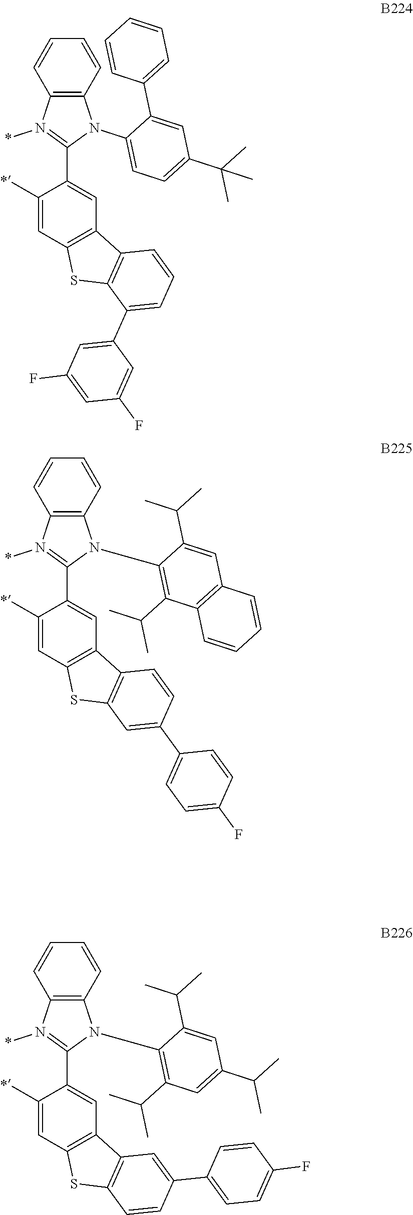

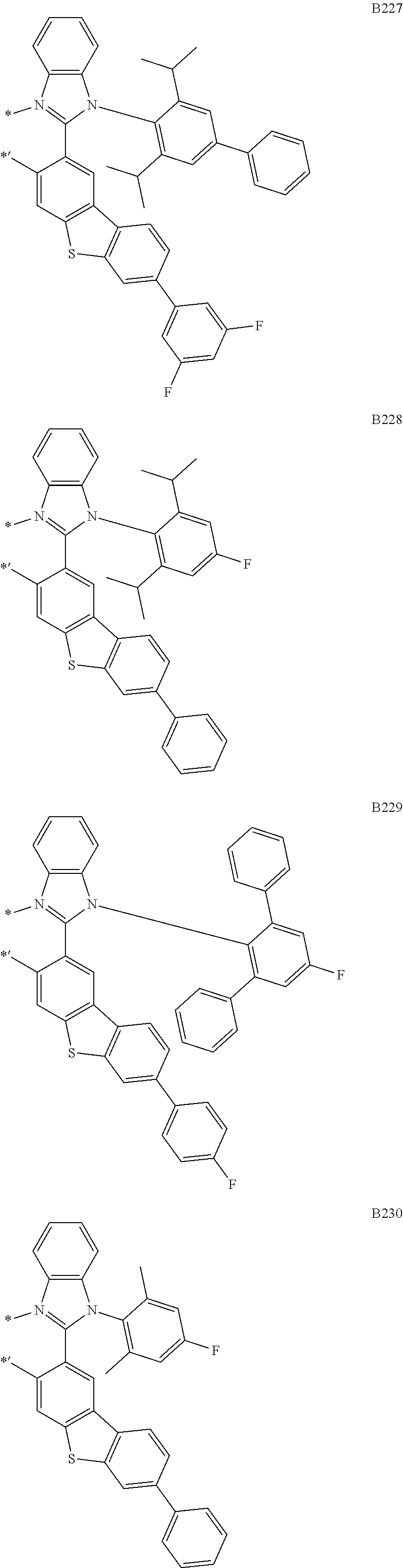

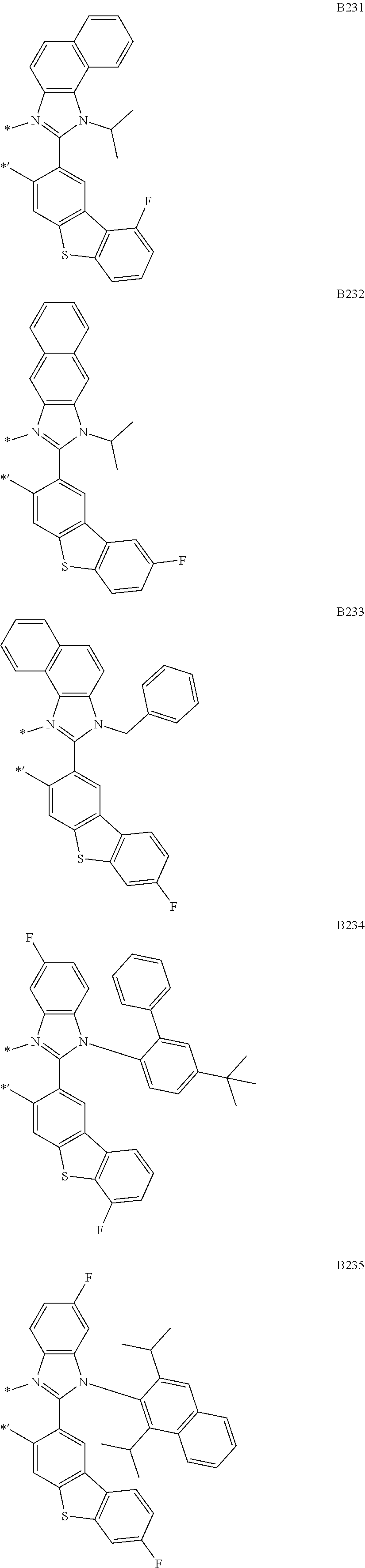

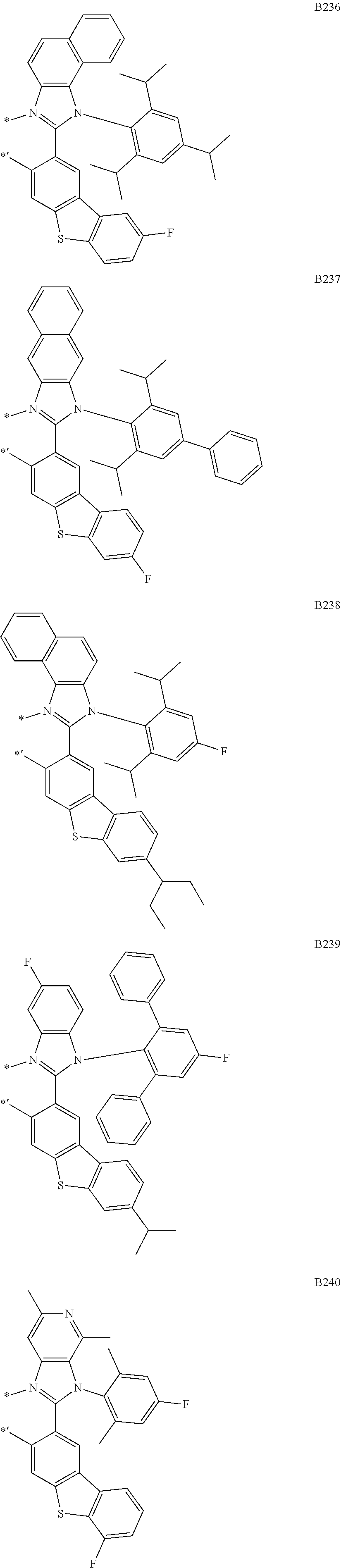

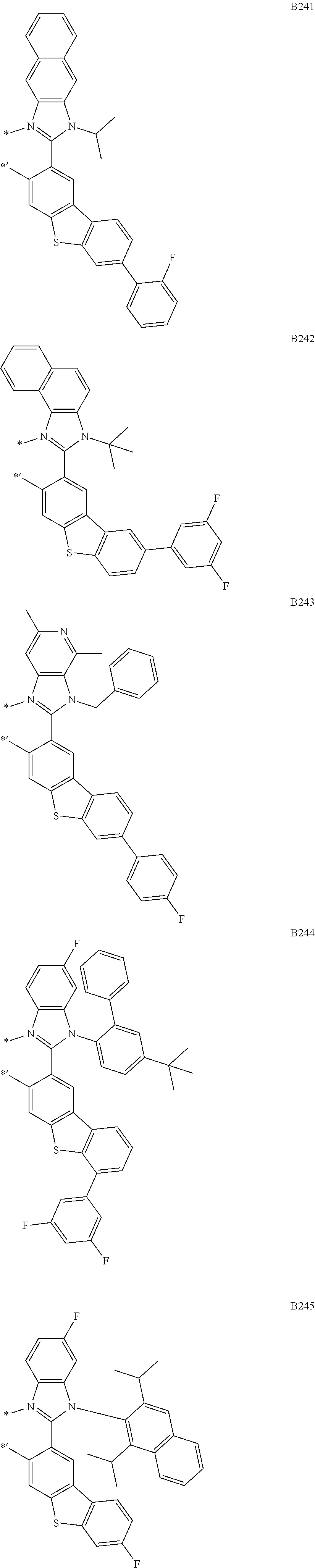

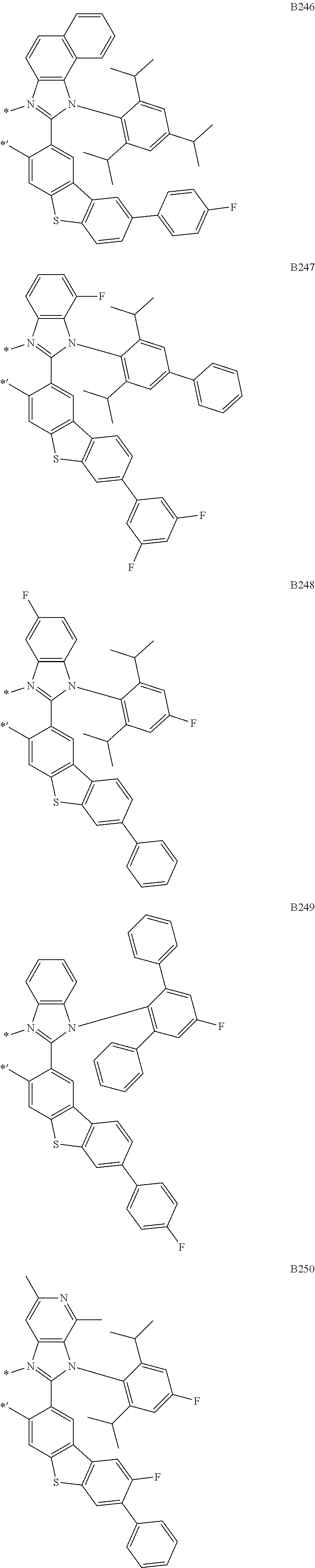

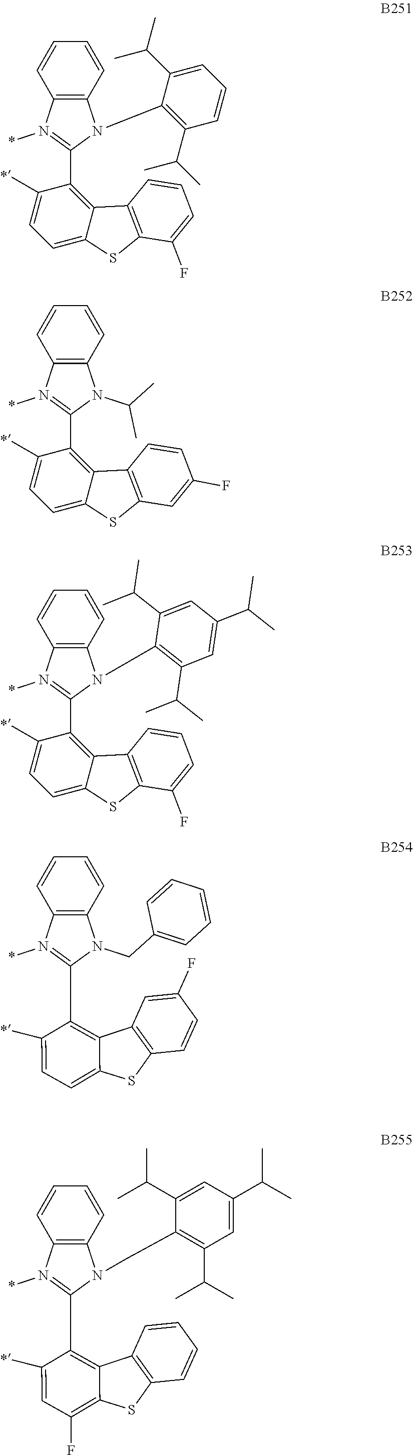

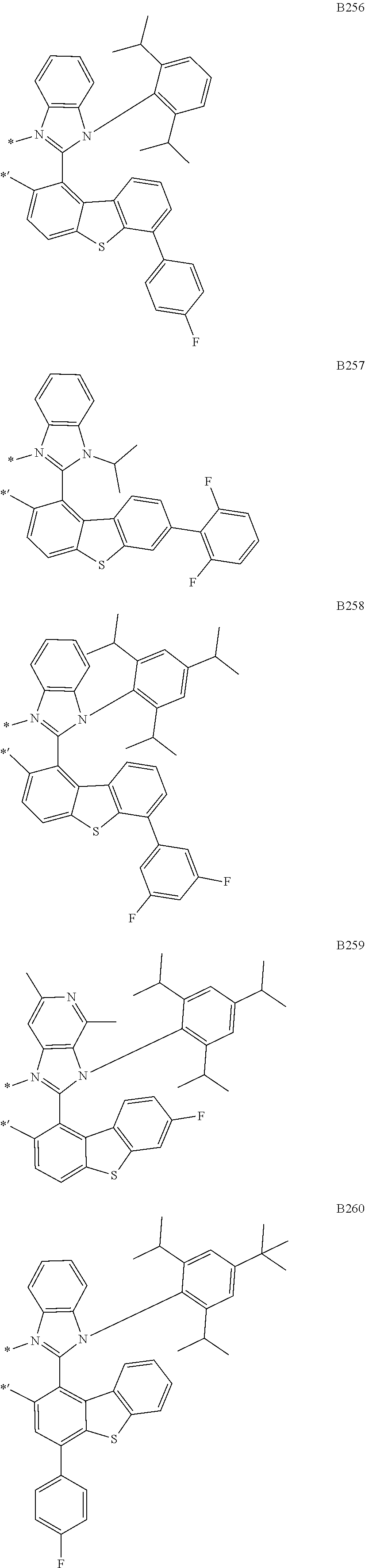

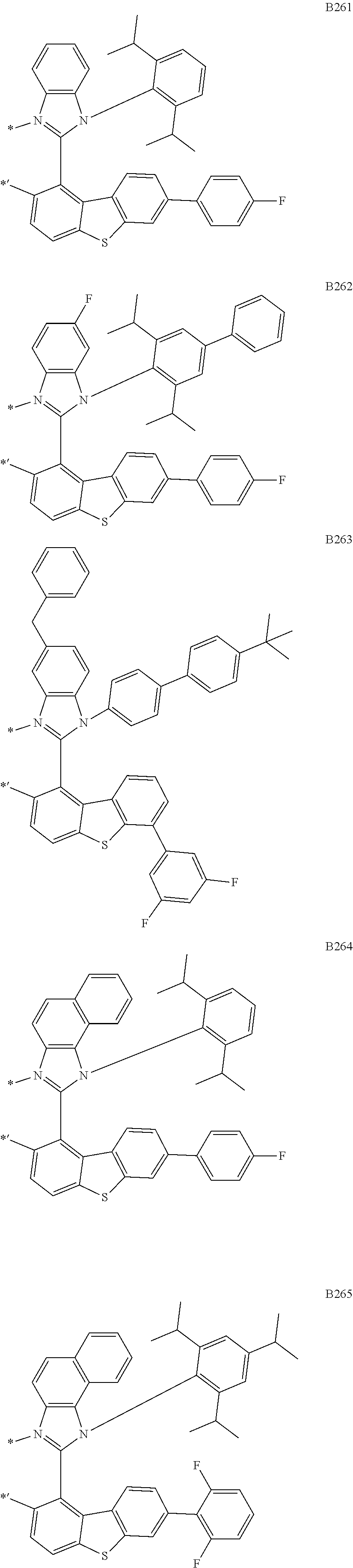

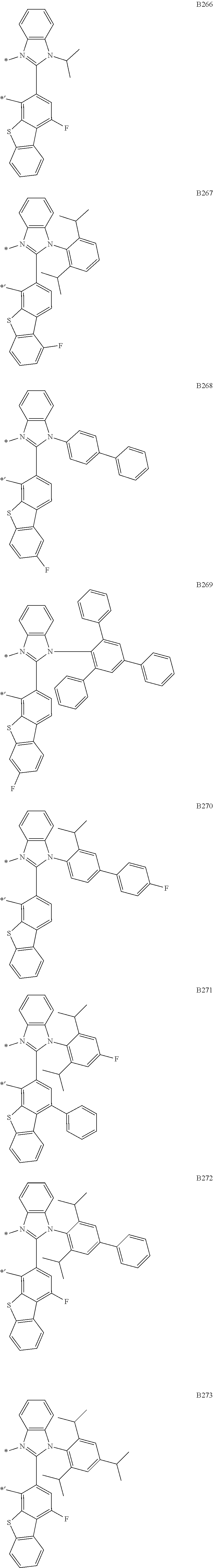

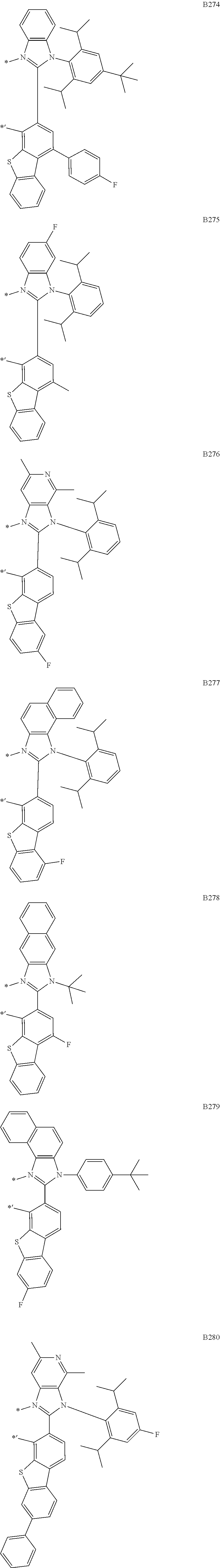

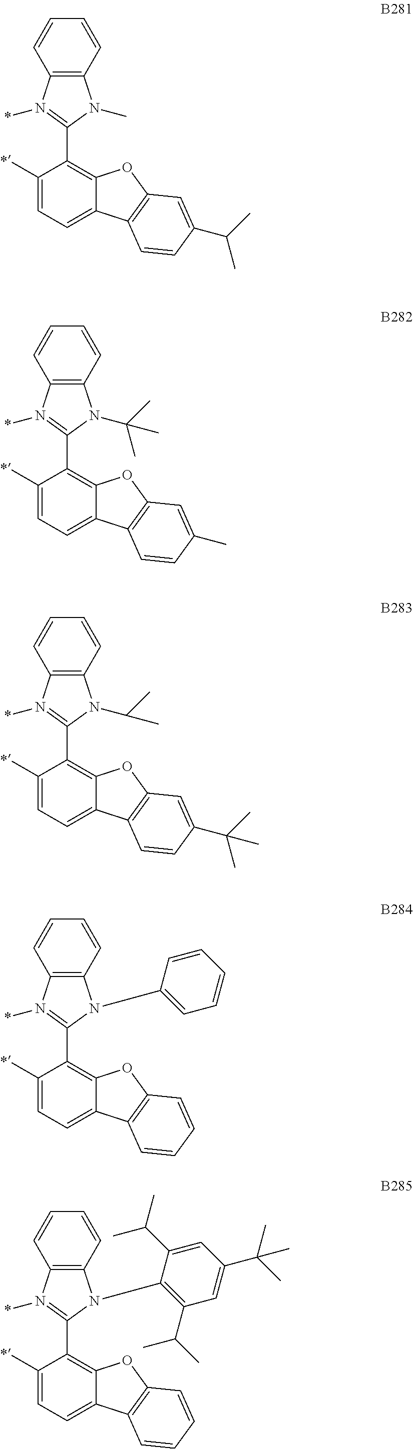

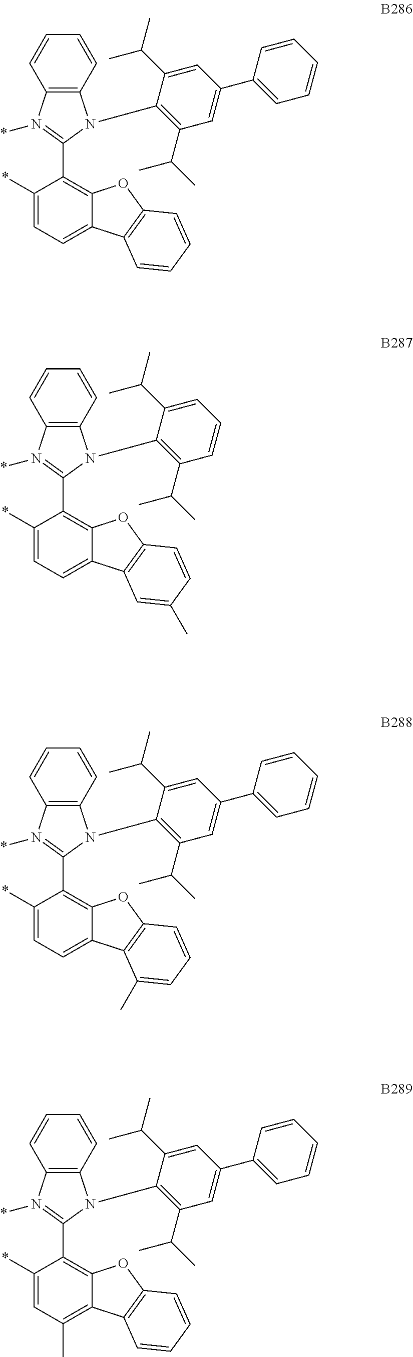

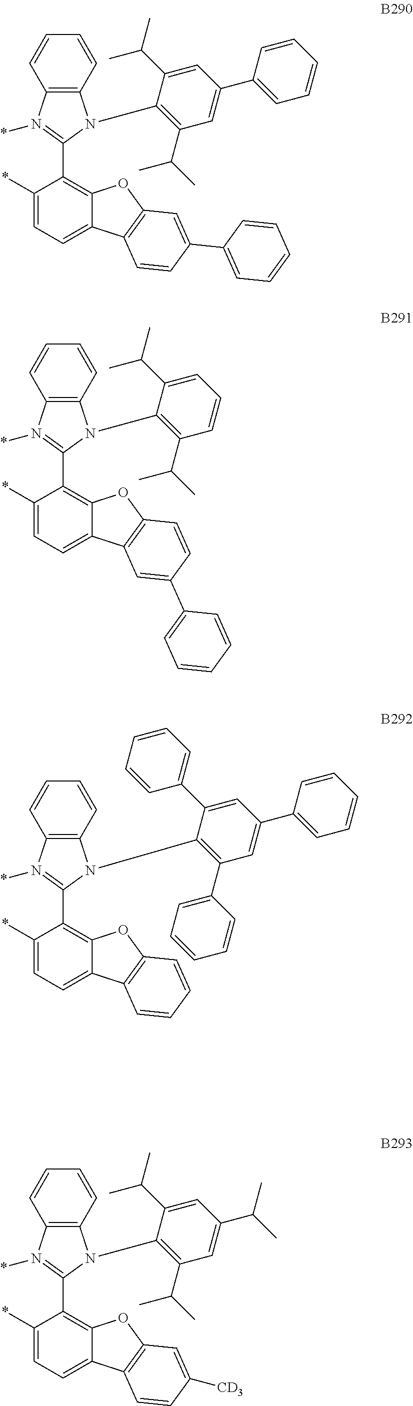

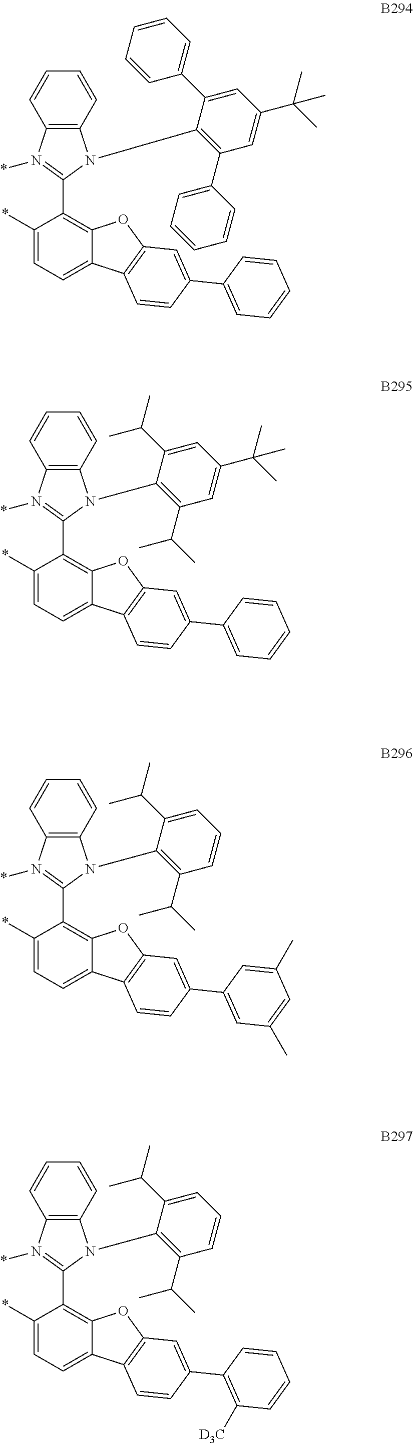

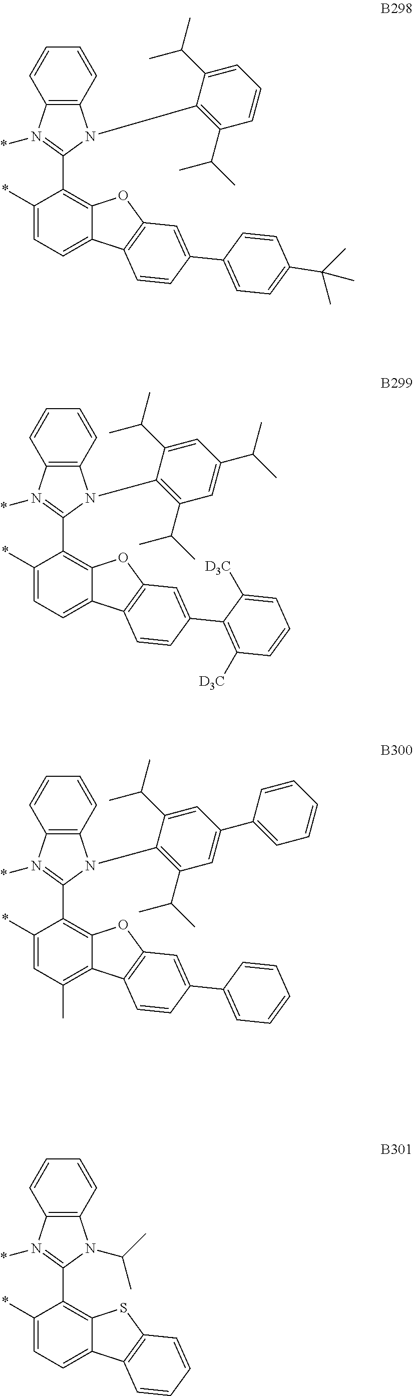

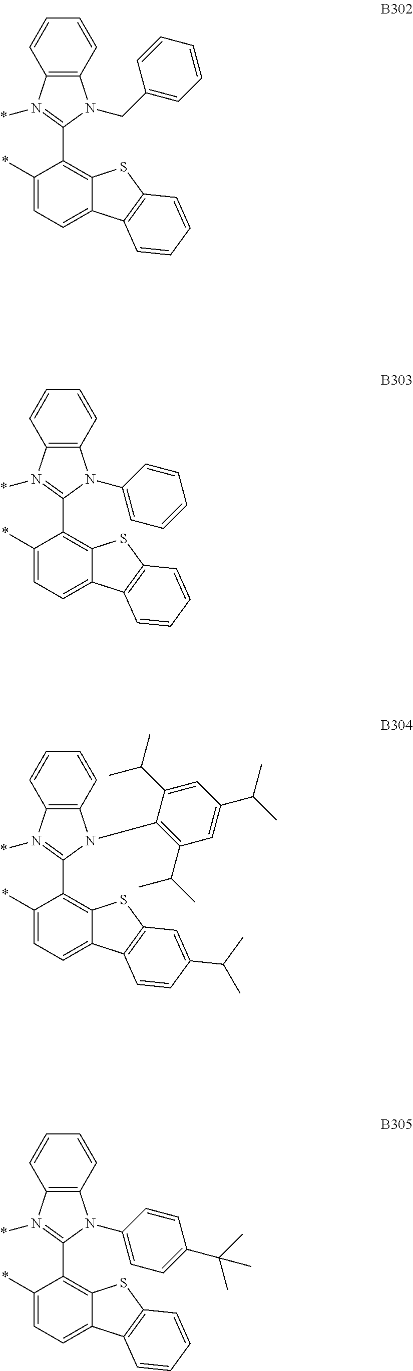

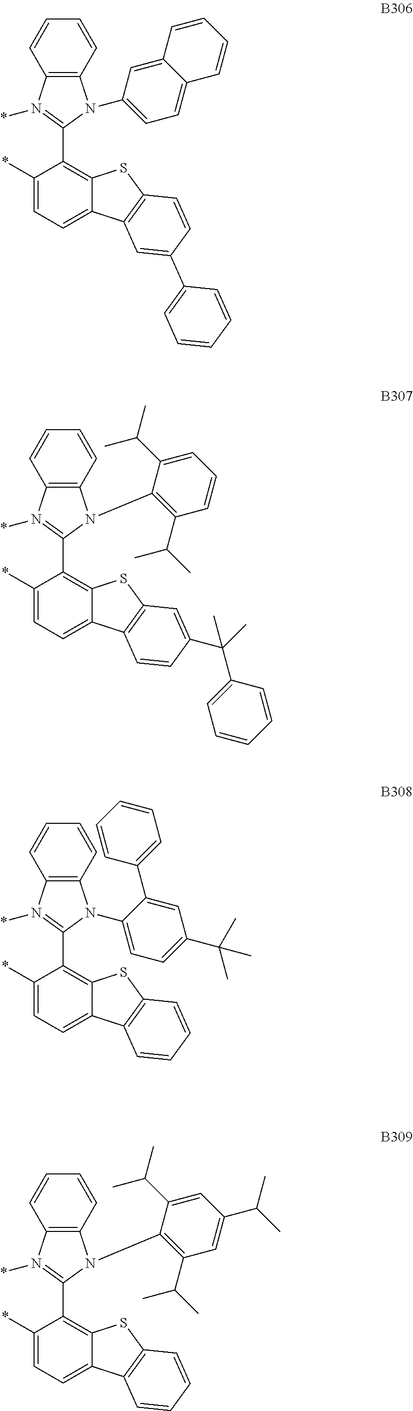

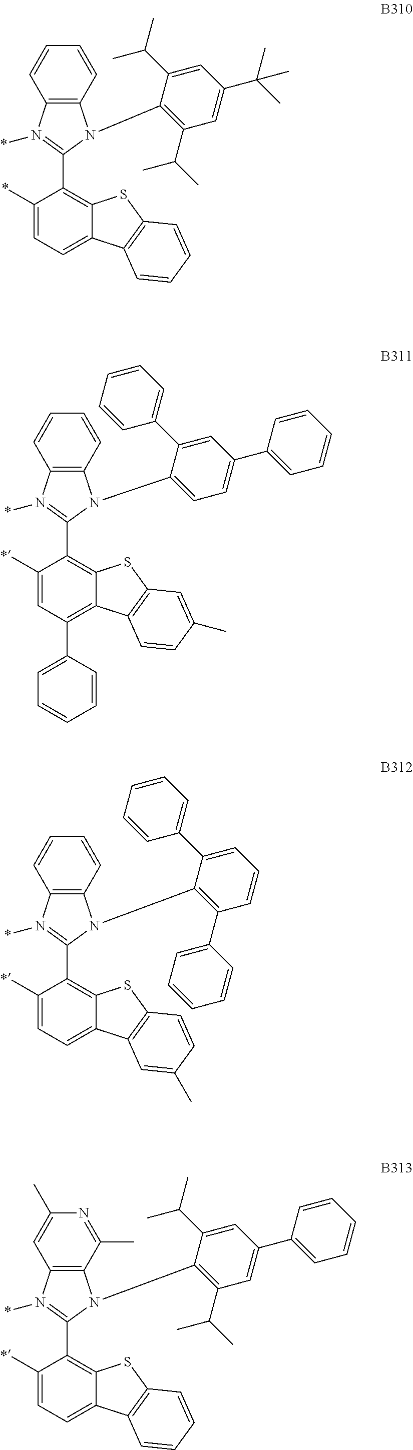

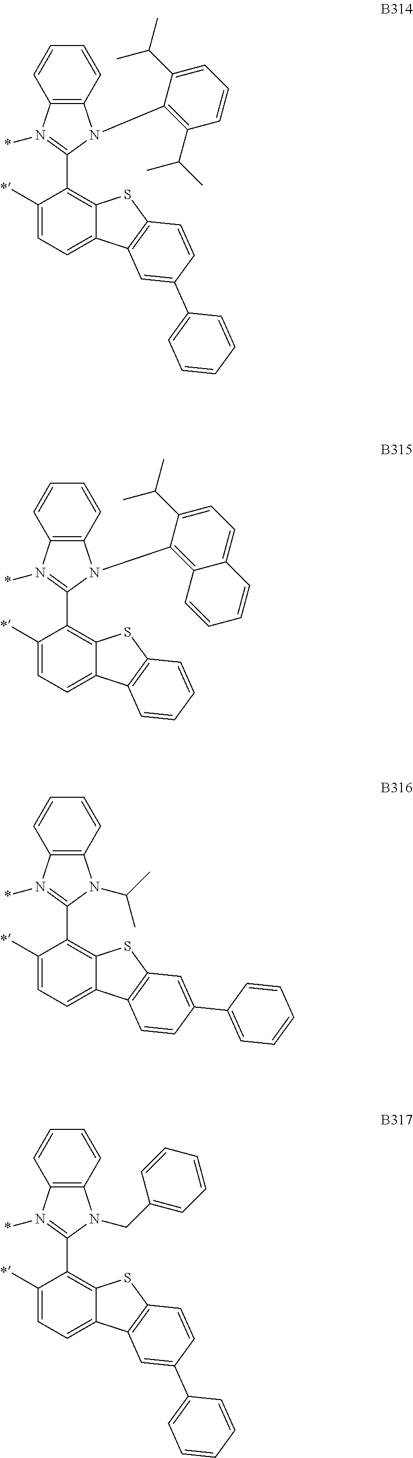

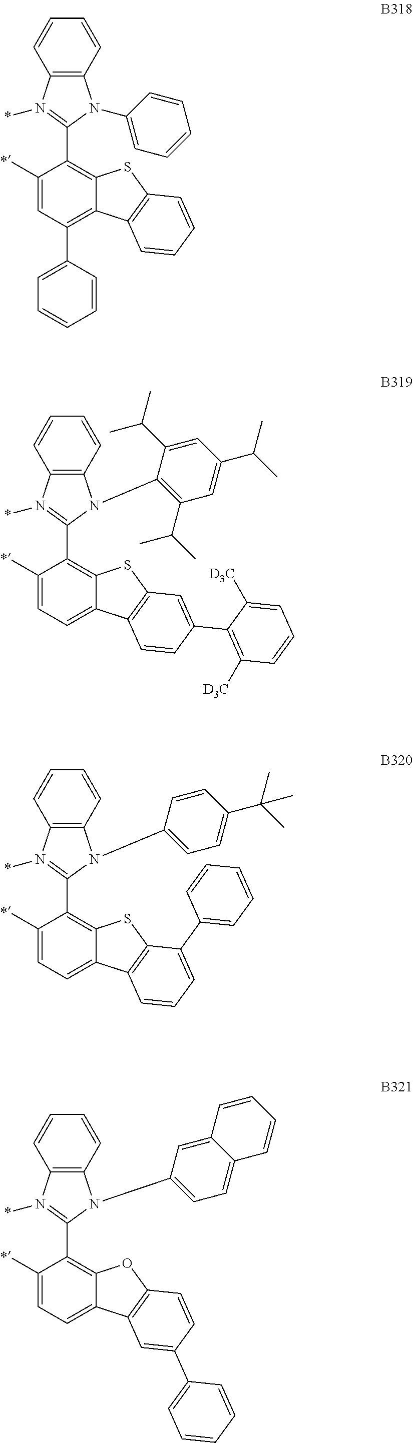

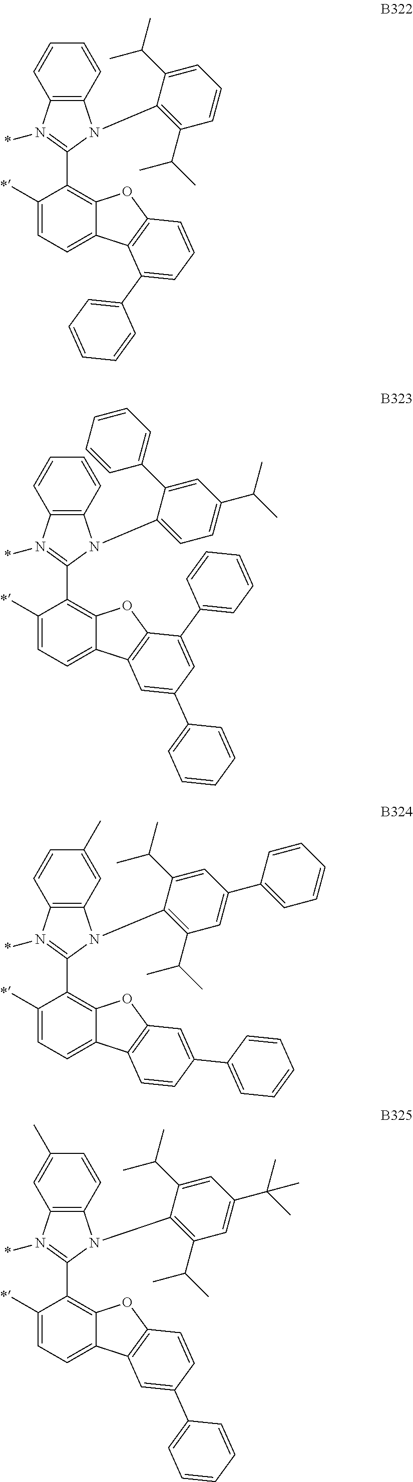

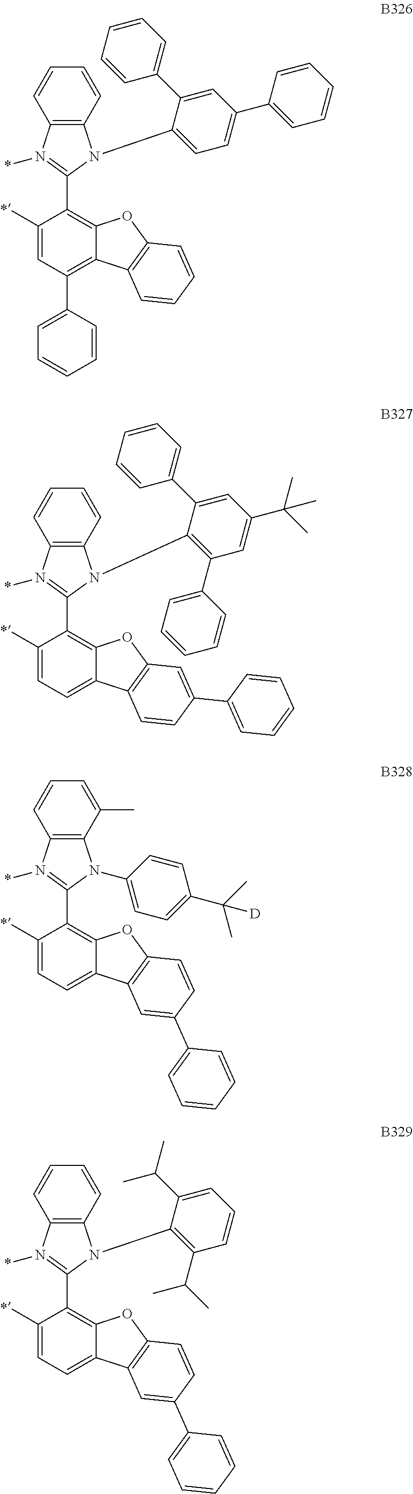

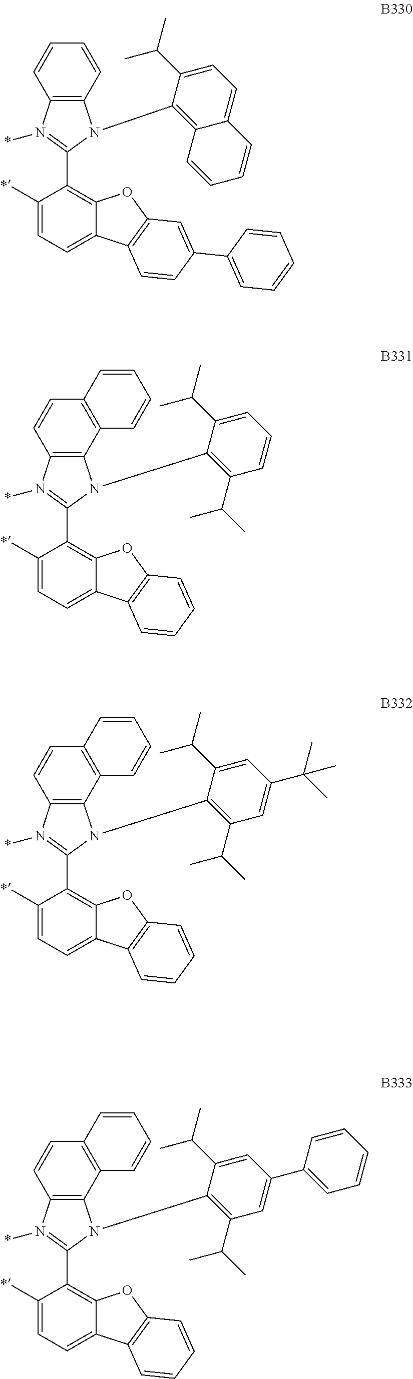

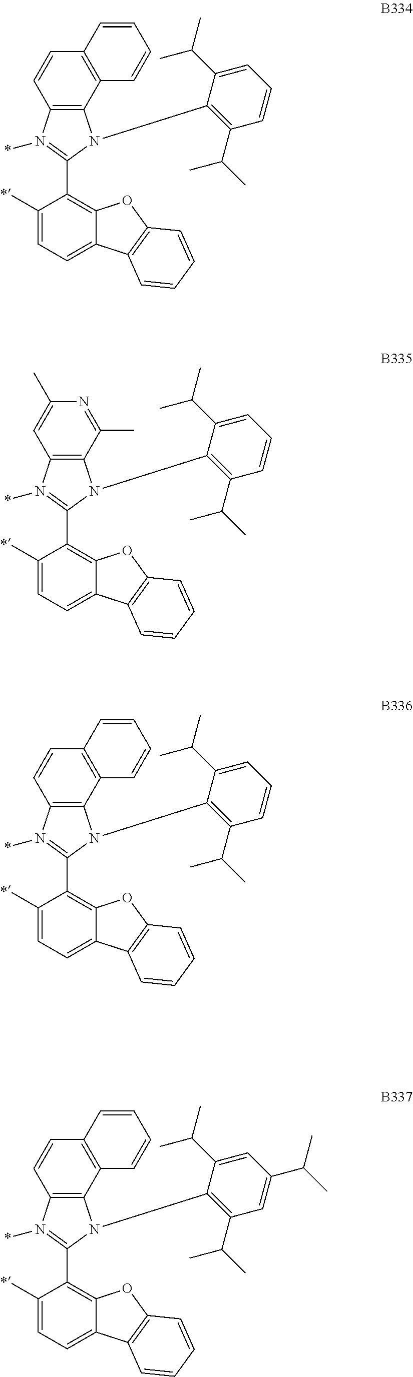

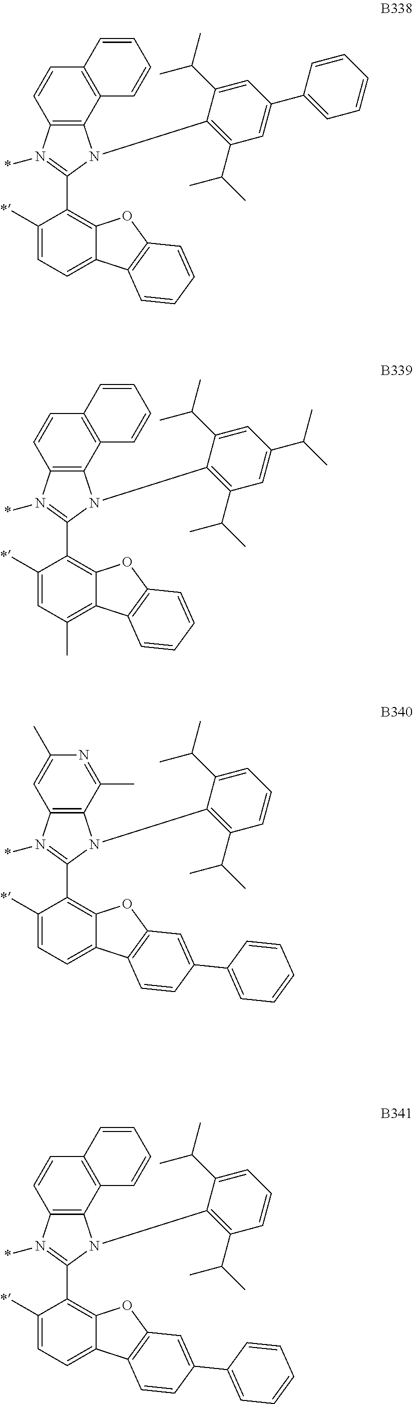

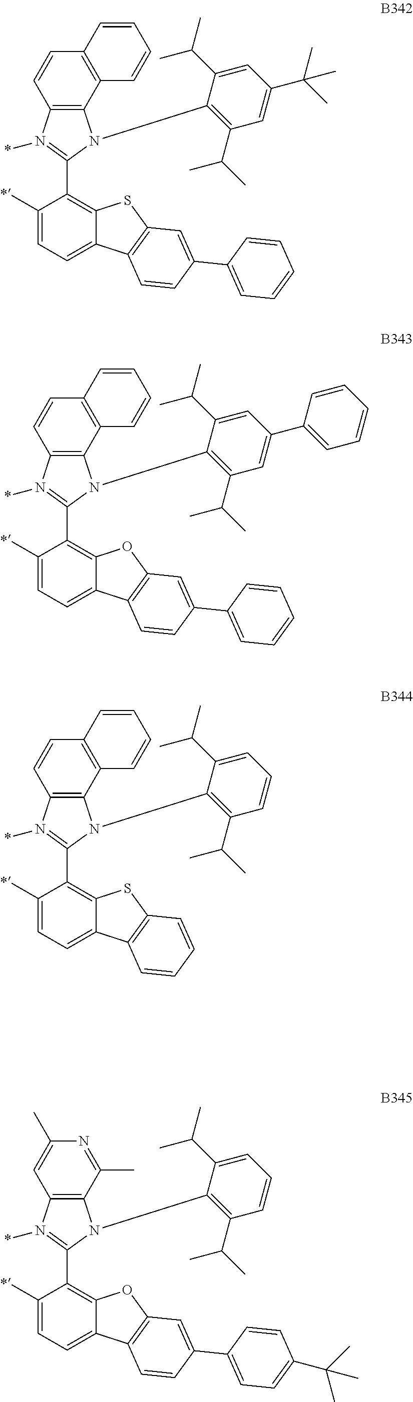

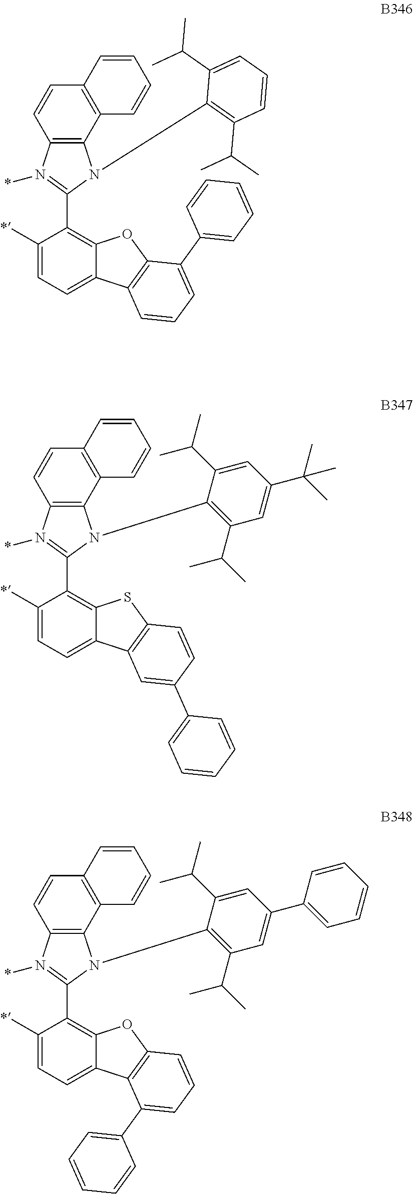

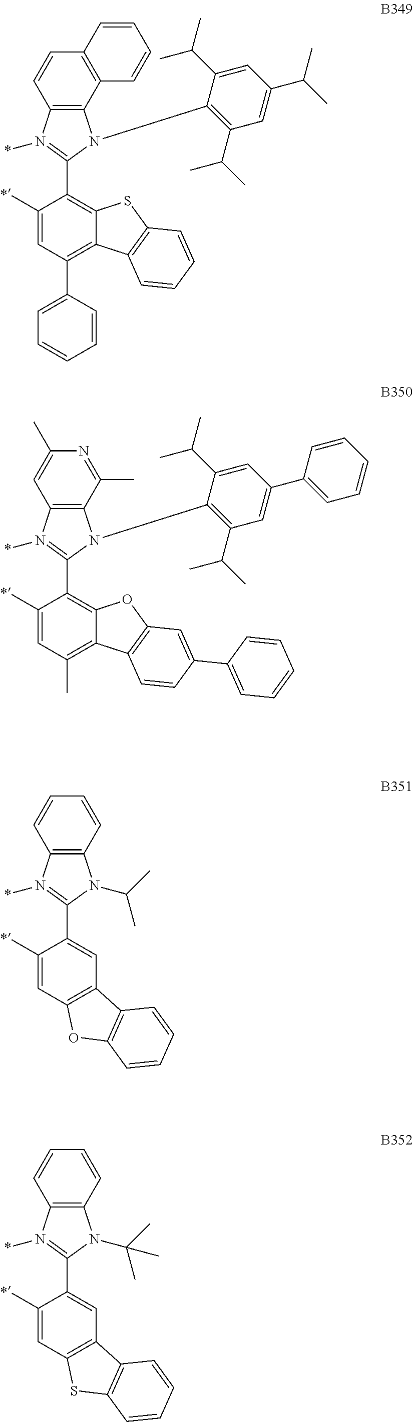

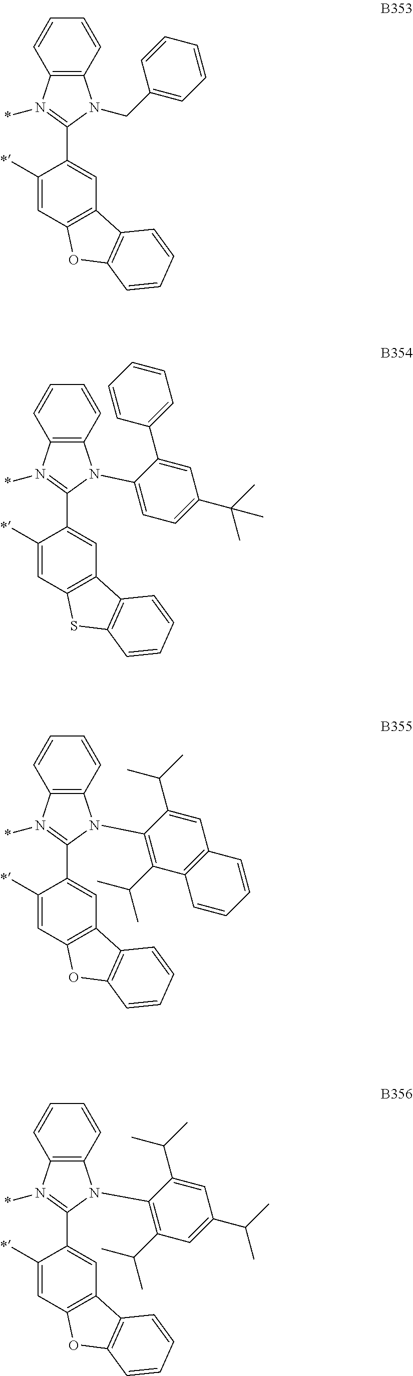

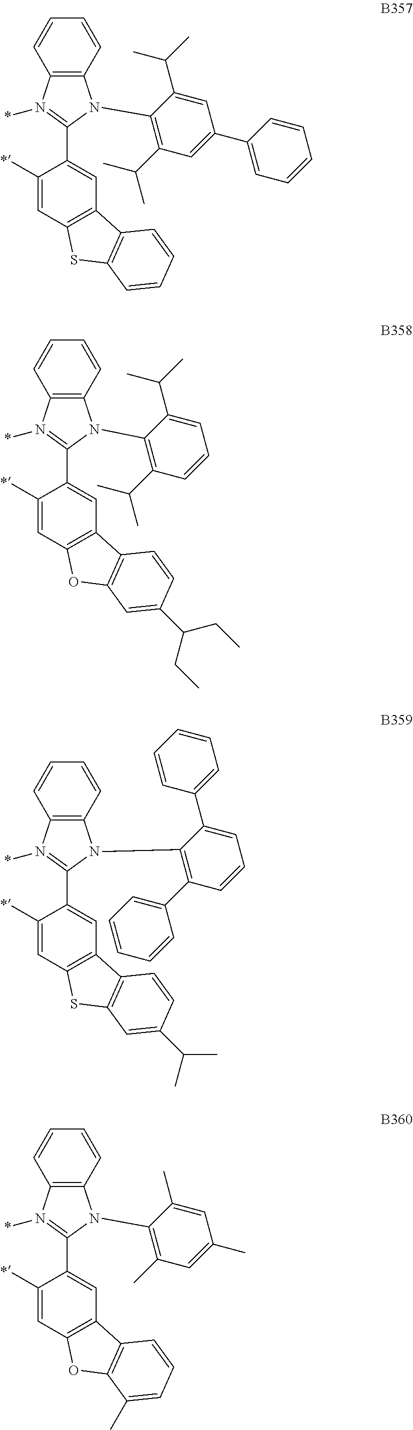

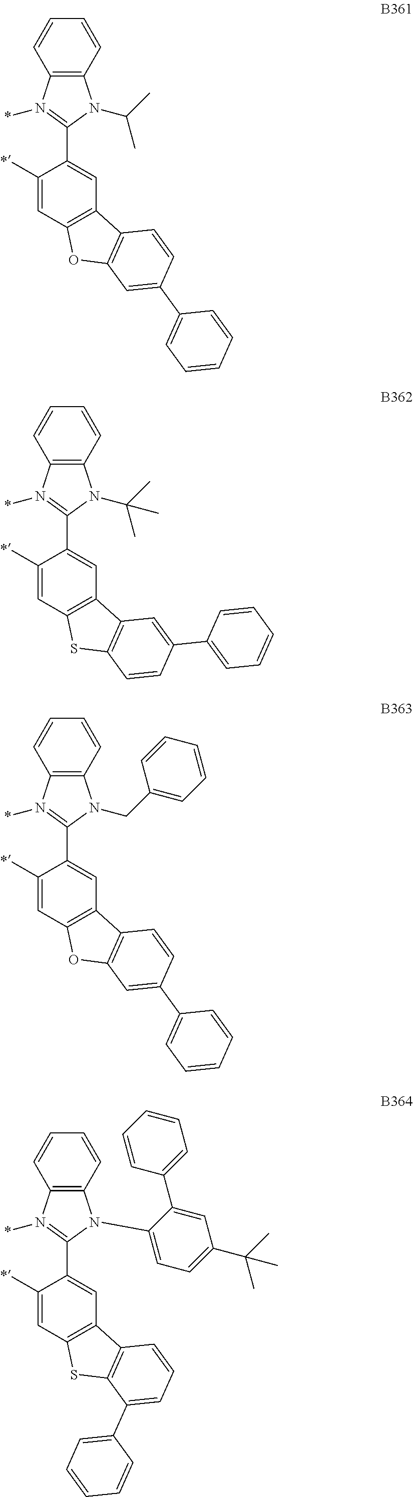

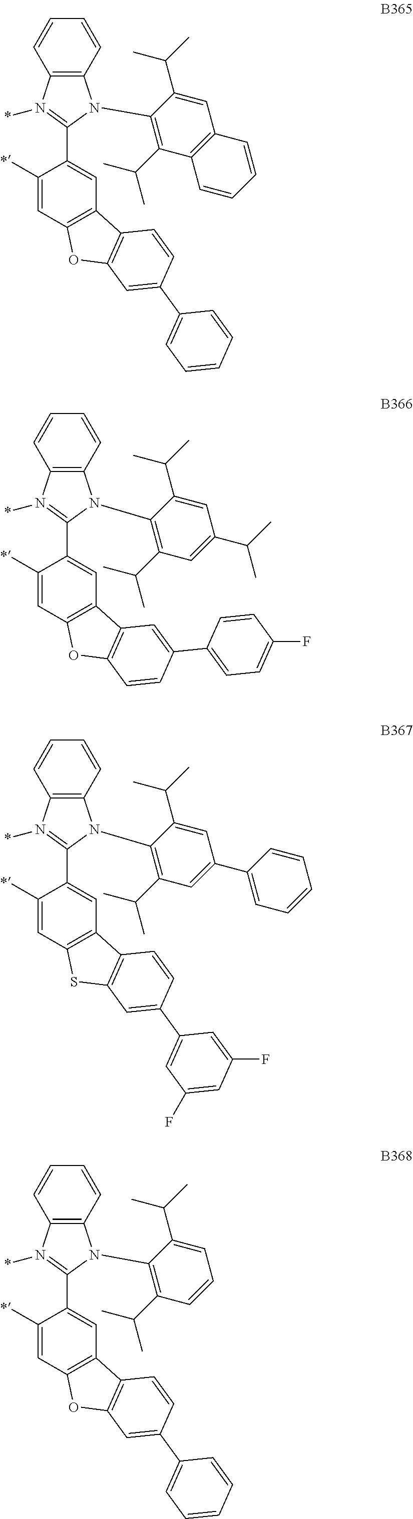

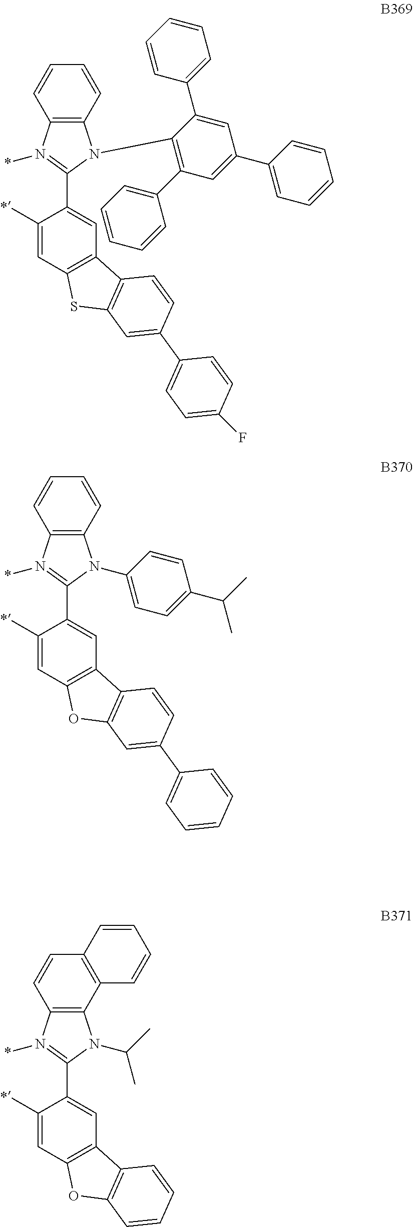

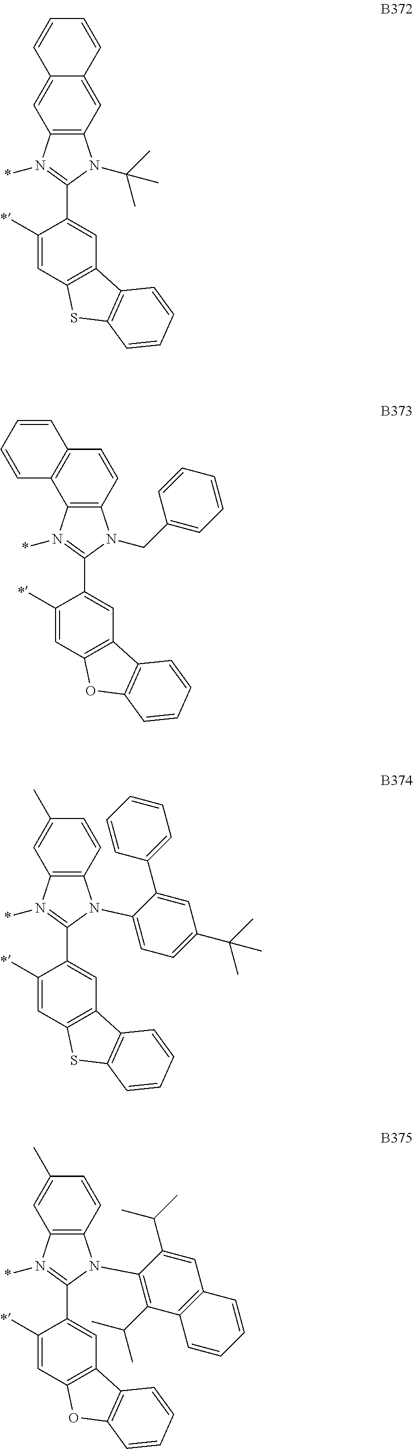

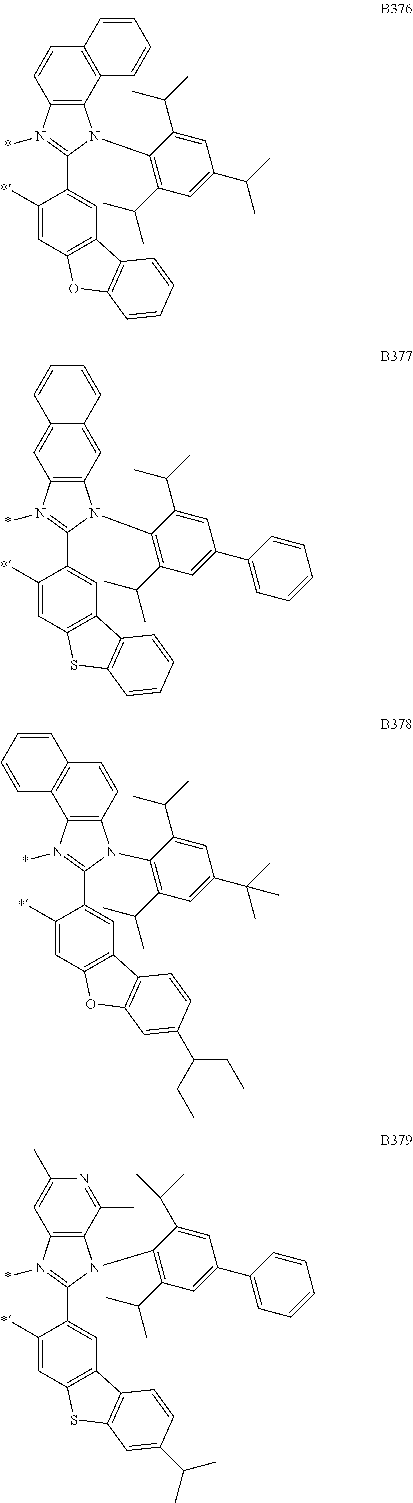

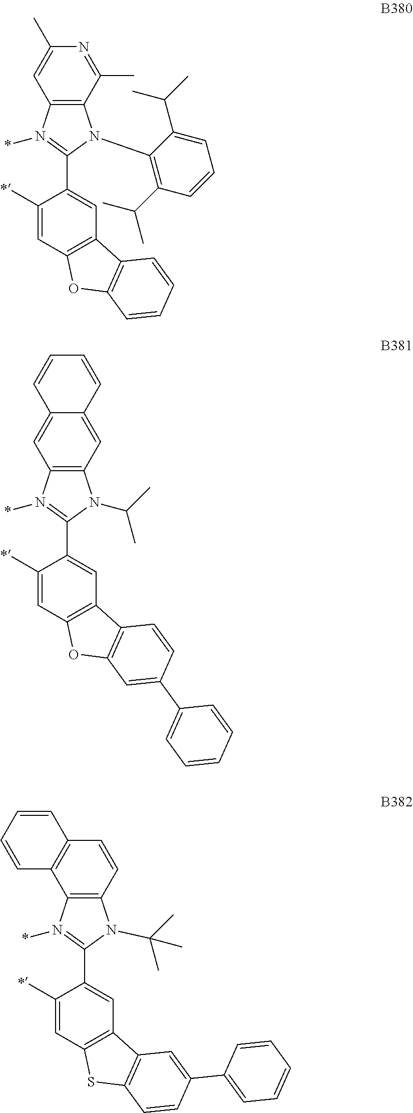

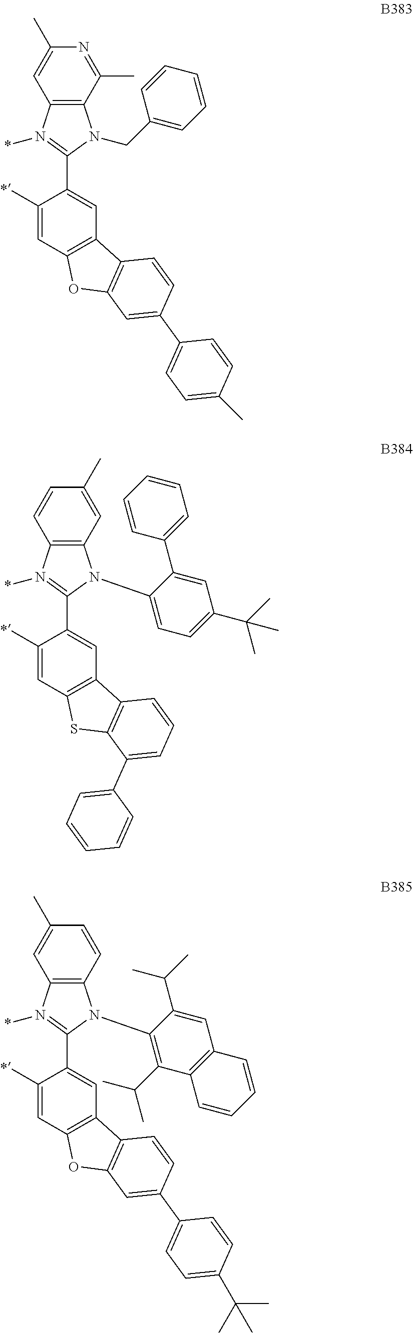

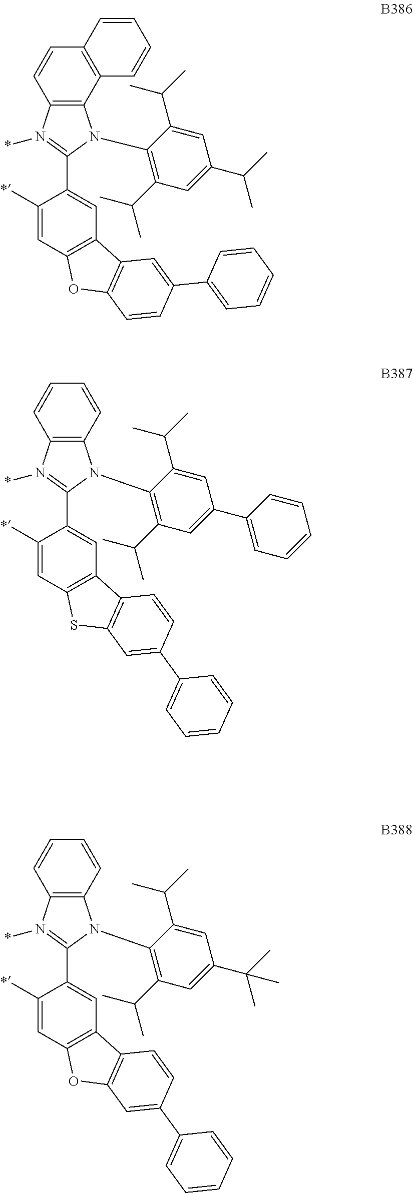

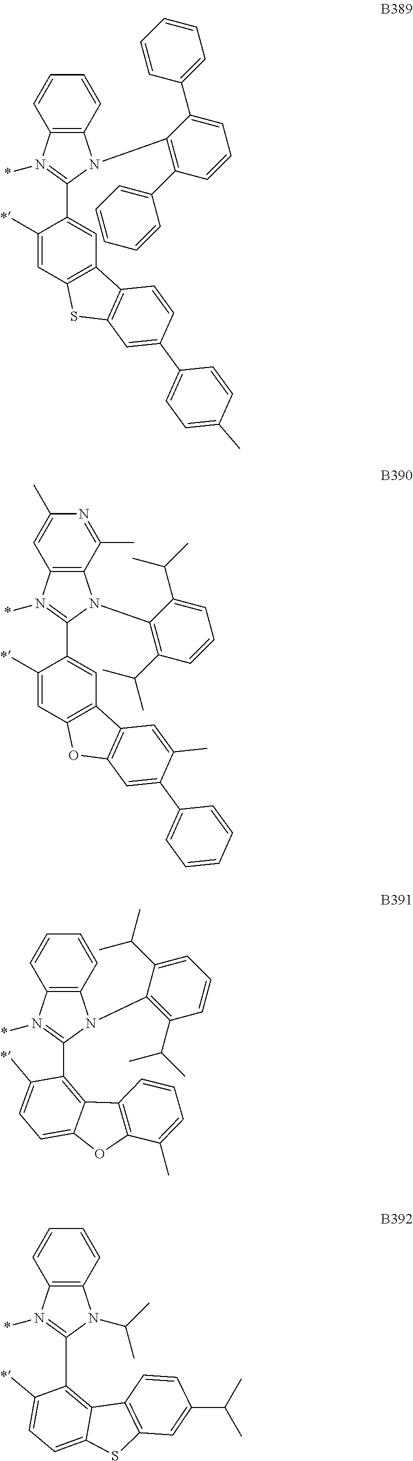

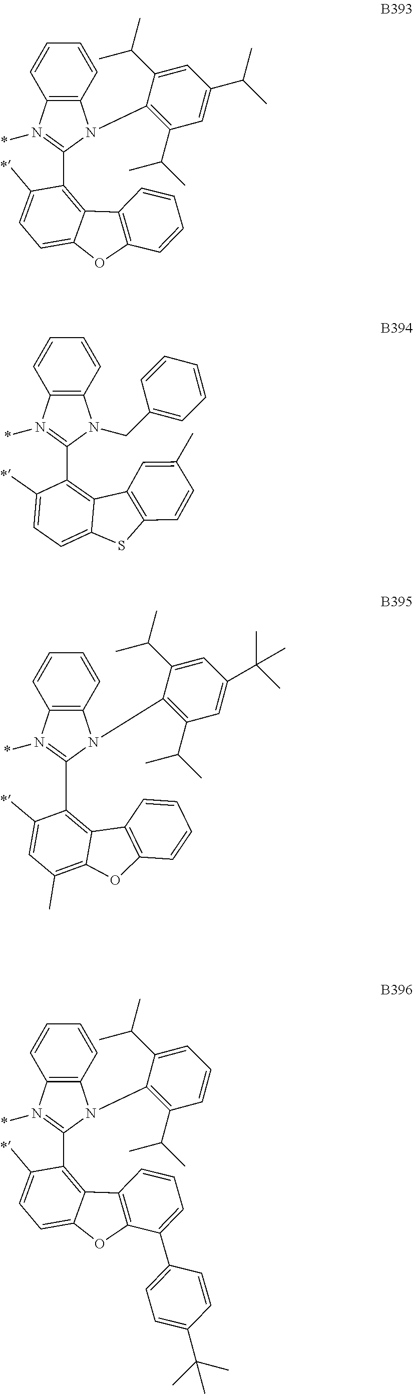

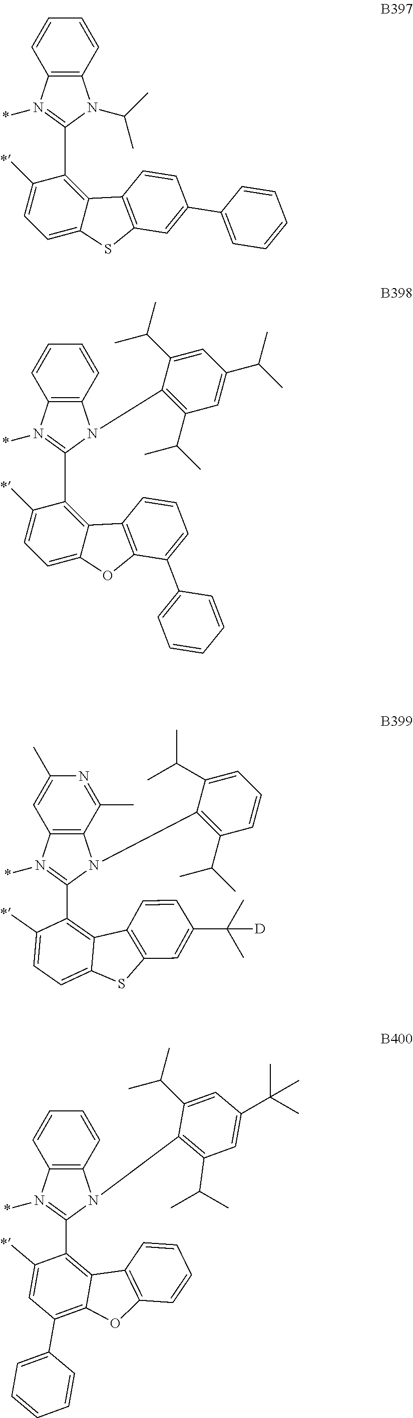

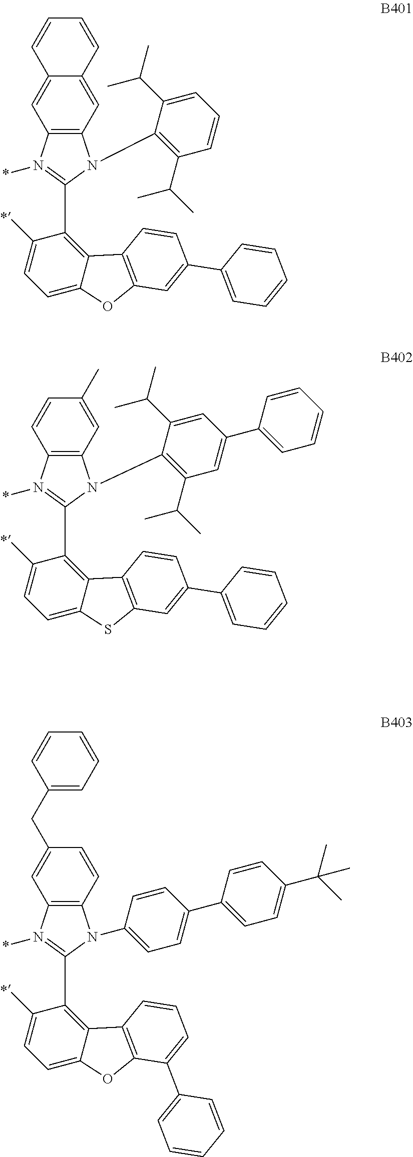

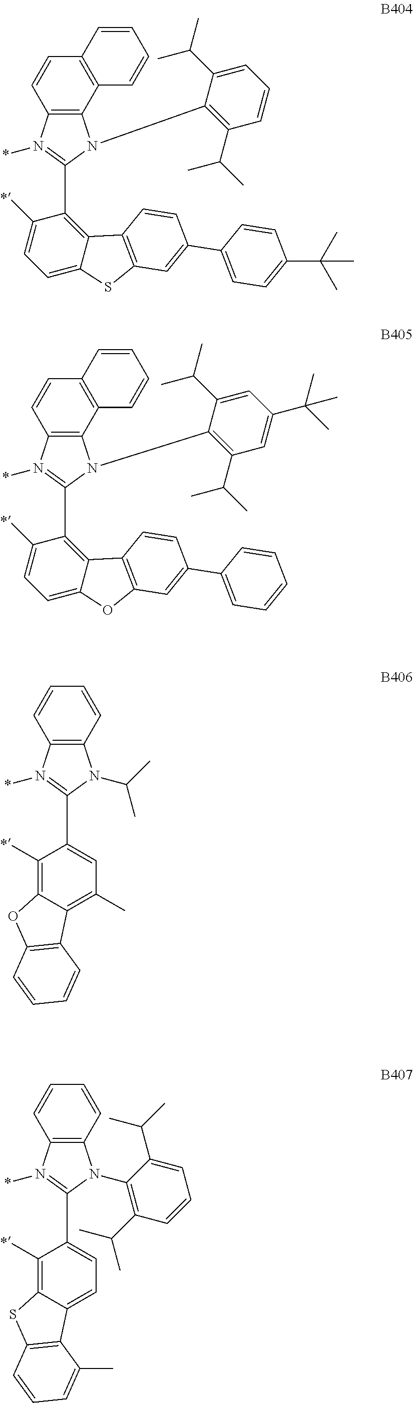

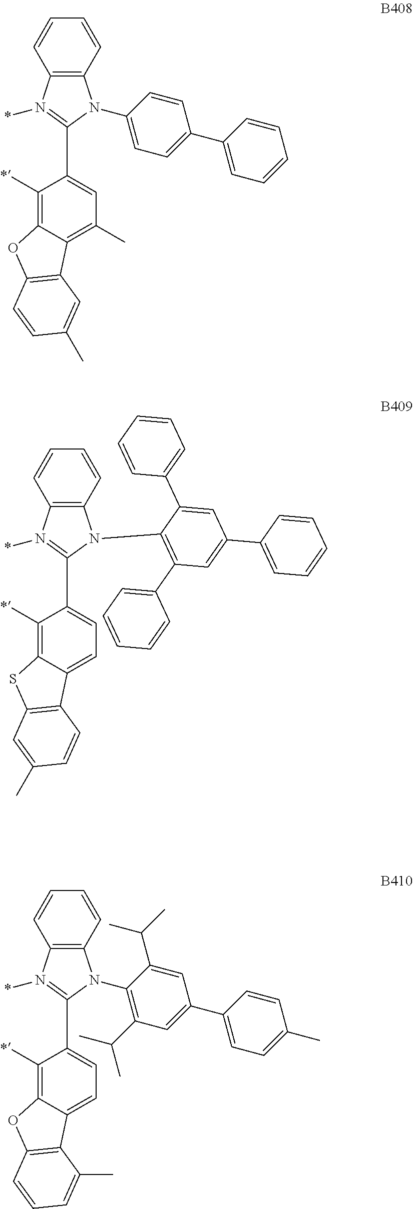

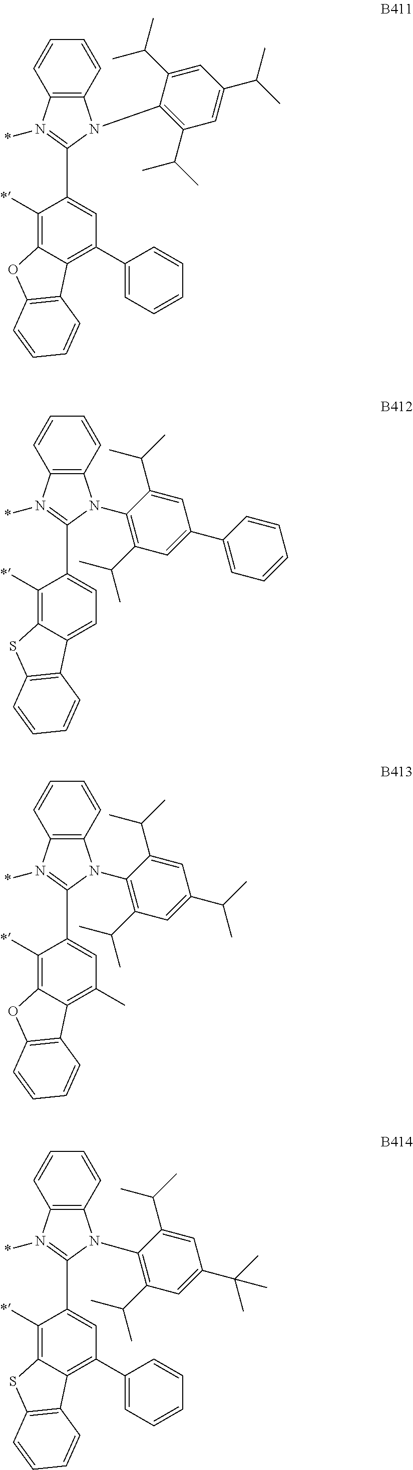

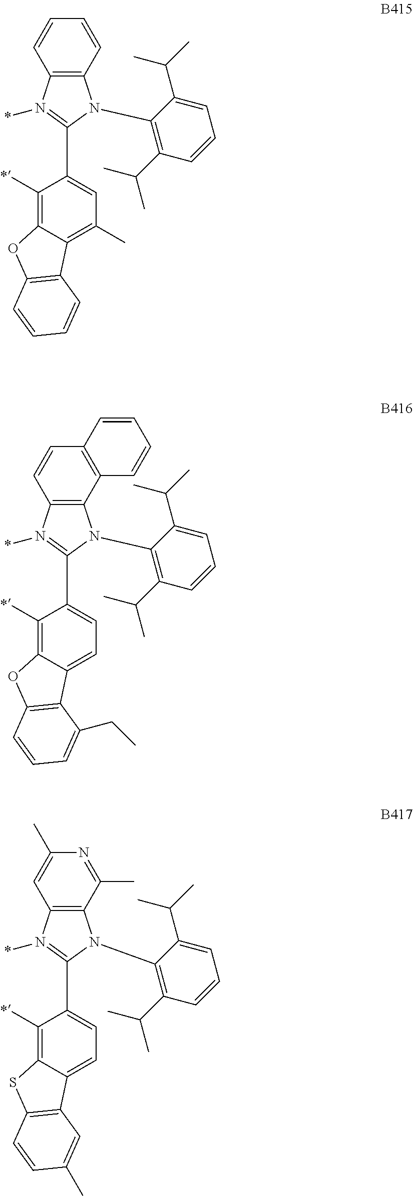

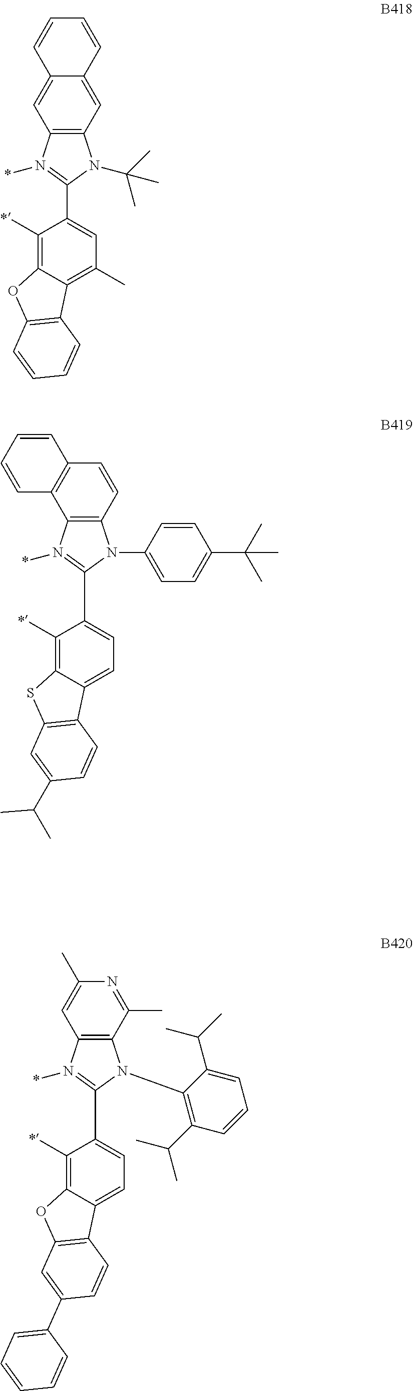

* and *′ in Formulae B1 to B420 each indicate a binding site to M₁ in Formula 1.

In one or more embodiments, the organometallic compound may be represented by Formula 1, wherein M₁ in Formula 1 may be iridium, and Ln₁, Ln₂, n1, and n2 may each be a compound as described in Tables 1 to 56.

TABLE 1

| Compound No. | Ln₁ | Ln₂ | n1 | n2 |
|---|---|---|---|---|
| 1-1 | A1 | B1 | 2 | 1 |
| 1-2 | A1 | B2 | 2 | 1 |
| 1-3 | A1 | B3 | 2 | 1 |
| 1-4 | A1 | B4 | 2 | 1 |
| 1-5 | A1 | B5 | 2 | 1 |
| 1-6 | A1 | B6 | 2 | 1 |
| 1-7 | A1 | B7 | 2 | 1 |
| 1-8 | A1 | B8 | 2 | 1 |
| 1-9 | A1 | B9 | 2 | 1 |
| 1-10 | A1 | B10 | 2 | 1 |
| 1-11 | A1 | B11 | 2 | 1 |
| 1-12 | A1 | B12 | 2 | 1 |
| 1-13 | A1 | B13 | 2 | 1 |
| 1-14 | A1 | B14 | 2 | 1 |
| 1-15 | A1 | B15 | 2 | 1 |
| 1-16 | A1 | B16 | 2 | 1 |
| 1-17 | A1 | B17 | 2 | 1 |
| 1-18 | A1 | B18 | 2 | 1 |
| 1-19 | A1 | B19 | 2 | 1 |
| 1-20 | A1 | B20 | 2 | 1 |
| 1-21 | A1 | B21 | 2 | 1 |
| 1-22 | A1 | B22 | 2 | 1 |
| 1-23 | A1 | B23 | 2 | 1 |
| 1-24 | A1 | B24 | 2 | 1 |
| 1-25 | A1 | B25 | 2 | 1 |
| 1-26 | A1 | B26 | 2 | 1 |
| 1-27 | A1 | B27 | 2 | 1 |
| 1-28 | A1 | B28 | 2 | 1 |
| 1-29 | A1 | B29 | 2 | 1 |
| 1-30 | A1 | B30 | 2 | 1 |

TABLE 2

| Compound No. | Ln₁ | Ln₂ | n1 | n2 |
|---|---|---|---|---|
| 1-31 | A1 | B31 | 2 | 1 |
| 1-32 | A1 | B32 | 2 | 1 |
| 1-33 | A1 | B33 | 2 | 1 |
| 1-34 | A1 | B34 | 2 | 1 |
| 1-35 | A1 | B35 | 2 | 1 |
| 1-36 | A1 | B36 | 2 | 1 |
| 1-37 | A1 | B37 | 2 | 1 |
| 1-38 | A1 | B38 | 2 | 1 |
| 1-39 | A1 | B39 | 2 | 1 |
| 1-40 | A1 | B40 | 2 | 1 |
| 1-41 | A1 | B41 | 2 | 1 |
| 1-42 | A1 | B42 | 2 | 1 |
| 1-43 | A1 | B43 | 2 | 1 |
| 1-44 | A1 | B44 | 2 | 1 |
| 1-45 | A1 | B45 | 2 | 1 |
| 1-46 | A1 | B46 | 2 | 1 |
| 1-47 | A1 | B47 | 2 | 1 |
| 1-48 | A1 | B48 | 2 | 1 |
| 1-49 | A1 | B49 | 2 | 1 |
| 1-50 | A1 | B50 | 2 | 1 |
| 1-51 | A1 | B51 | 2 | 1 |
| 1-52 | A1 | B52 | 2 | 1 |
| 1-53 | A1 | B53 | 2 | 1 |
| 1-54 | A1 | B54 | 2 | 1 |
| 1-55 | A1 | B55 | 2 | 1 |
| 1-56 | A1 | B56 | 2 | 1 |
| 1-57 | A1 | B57 | 2 | 1 |
| 1-58 | A1 | B58 | 2 | 1 |
| 1-59 | A1 | B59 | 2 | 1 |
| 1-60 | A1 | B60 | 2 | 1 |

TABLE 3

| Compound No. | Ln₁ | Ln₂ | n1 | n2 |
|---|---|---|---|---|
| 1-61 | A1 | B61 | 2 | 1 |
| 1-62 | A1 | B62 | 2 | 1 |
| 1-63 | A1 | B63 | 2 | 1 |
| 1-64 | A1 | B64 | 2 | 1 |
| 1-65 | A1 | B65 | 2 | 1 |
| 1-66 | A1 | B66 | 2 | 1 |
| 1-67 | A1 | B67 | 2 | 1 |
| 1-68 | A1 | B68 | 2 | 1 |
| 1-69 | A1 | B69 | 2 | 1 |
| 1-70 | A1 | B70 | 2 | 1 |
| 1-71 | A1 | B71 | 2 | 1 |
| 1-72 | A1 | B72 | 2 | 1 |
| 1-73 | A1 | B73 | 2 | 1 |
| 1-74 | A1 | B74 | 2 | 1 |
| 1-75 | A1 | B75 | 2 | 1 |
| 1-76 | A1 | B76 | 2 | 1 |
| 1-77 | A1 | B77 | 2 | 1 |
| 1-78 | A1 | B78 | 2 | 1 |
| 1-79 | A1 | B79 | 2 | 1 |
| 1-80 | A1 | B80 | 2 | 1 |
| 1-81 | A1 | B81 | 2 | 1 |
| 1-82 | A1 | B82 | 2 | 1 |
| 1-83 | A1 | B83 | 2 | 1 |
| 1-84 | A1 | B84 | 2 | 1 |
| 1-85 | A1 | B85 | 2 | 1 |
| 1-86 | A1 | B86 | 2 | 1 |
| 1-87 | A1 | B87 | 2 | 1 |
| 1-88 | A1 | B88 | 2 | 1 |
| 1-89 | A1 | B89 | 2 | 1 |
| 1-90 | A1 | B90 | 2 | 1 |

TABLE 4

| Compound No. | Ln₁ | Ln₂ | n1 | n2 |
|---|---|---|---|---|
| 1-91 | A1 | B91 | 2 | 1 |
| 1-92 | A1 | B92 | 2 | 1 |
| 1-93 | A1 | B93 | 2 | 1 |
| 1-94 | A1 | B94 | 2 | 1 |
| 1-95 | A1 | B95 | 2 | 1 |
| 1-96 | A1 | B96 | 2 | 1 |
| 1-97 | A1 | B97 | 2 | 1 |
| 1-98 | A1 | B98 | 2 | 1 |
| 1-99 | A1 | B99 | 2 | 1 |
| 1-100 | A1 | B100 | 2 | 1 |
| 1-101 | A1 | B101 | 2 | 1 |
| 1-102 | A1 | B102 | 2 | 1 |
| 1-103 | A1 | B103 | 2 | 1 |
| 1-104 | A1 | B104 | 2 | 1 |
| 1-105 | A1 | B105 | 2 | 1 |
| 1-106 | A1 | B106 | 2 | 1 |
| 1-107 | A1 | B107 | 2 | 1 |
| 1-108 | A1 | B108 | 2 | 1 |
| 1-109 | A1 | B109 | 2 | 1 |
| 1-110 | A1 | B110 | 2 | 1 |
| 1-111 | A1 | B111 | 2 | 1 |
| 1-112 | A1 | B112 | 2 | 1 |
| 1-113 | A1 | B113 | 2 | 1 |
| 1-114 | A1 | B114 | 2 | 1 |
| 1-115 | A1 | B115 | 2 | 1 |
| 1-116 | A1 | B116 | 2 | 1 |
| 1-117 | A1 | B117 | 2 | 1 |
| 1-118 | A1 | B118 | 2 | 1 |
| 1-119 | A1 | B119 | 2 | 1 |
| 1-120 | A1 | B120 | 2 | 1 |

TABLE 5

| Compound No. | Ln₁ | Ln₂ | n1 | n2 |
|---|---|---|---|---|
| 1-121 | A1 | B121 | 2 | 1 |
| 1-122 | A1 | B122 | 2 | 1 |
| 1-123 | A1 | B123 | 2 | 1 |
| 1-124 | A1 | B124 | 2 | 1 |
| 1-125 | A1 | B125 | 2 | 1 |
| 1-126 | A1 | B126 | 2 | 1 |
| 1-127 | A1 | B127 | 2 | 1 |
| 1-128 | A1 | B128 | 2 | 1 |
| 1-129 | A1 | B129 | 2 | 1 |
| 1-130 | A1 | B130 | 2 | 1 |
| 1-131 | A1 | B131 | 2 | 1 |
| 1-132 | A1 | B132 | 2 | 1 |
| 1-133 | A1 | B133 | 2 | 1 |
| 1-134 | A1 | B134 | 2 | 1 |
| 1-135 | A1 | B135 | 2 | 1 |
| 1-136 | A1 | B136 | 2 | 1 |
| 1-137 | A1 | B137 | 2 | 1 |
| 1-138 | A1 | B138 | 2 | 1 |
| 1-139 | A1 | B139 | 2 | 1 |
| 1-140 | A1 | B140 | 2 | 1 |
| 1-141 | A1 | B141 | 2 | 1 |
| 1-142 | A1 | B142 | 2 | 1 |
| 1-143 | A1 | B143 | 2 | 1 |
| 1-144 | A1 | B144 | 2 | 1 |
| 1-145 | A1 | B145 | 2 | 1 |
| 1-146 | A1 | B146 | 2 | 1 |
| 1-147 | A1 | B147 | 2 | 1 |
| 1-148 | A1 | B148 | 2 | 1 |
| 1-149 | A1 | B149 | 2 | 1 |
| 1-150 | A1 | B150 | 2 | 1 |

TABLE 6

| Compound No. | Ln₁ | Ln₂ | n1 | n2 |
|---|---|---|---|---|
| 1-151 | A1 | B151 | 2 | 1 |
| 1-152 | A1 | B152 | 2 | 1 |
| 1-153 | A1 | B153 | 2 | 1 |
| 1-154 | A1 | B154 | 2 | 1 |
| 1-155 | A1 | B155 | 2 | 1 |
| 1-156 | A1 | B156 | 2 | 1 |
| 1-157 | A1 | B157 | 2 | 1 |
| 1-158 | A1 | B158 | 2 | 1 |
| 1-159 | A1 | B159 | 2 | 1 |
| 1-160 | A1 | B160 | 2 | 1 |
| 1-161 | A1 | B161 | 2 | 1 |
| 1-162 | A1 | B162 | 2 | 1 |
| 1-163 | A1 | B163 | 2 | 1 |
| 1-164 | A1 | B164 | 2 | 1 |
| 1-165 | A1 | B165 | 2 | 1 |
| 1-166 | A1 | B166 | 2 | 1 |
| 1-167 | A1 | B167 | 2 | 1 |
| 1-168 | A1 | B168 | 2 | 1 |
| 1-169 | A1 | B169 | 2 | 1 |
| 1-170 | A1 | B170 | 2 | 1 |
| 1-171 | A1 | B171 | 2 | 1 |
| 1-172 | A1 | B172 | 2 | 1 |
| 1-173 | A1 | B173 | 2 | 1 |
| 1-174 | A1 | B174 | 2 | 1 |
| 1-175 | A1 | B175 | 2 | 1 |
| 1-176 | A1 | B176 | 2 | 1 |
| 1-177 | A1 | B177 | 2 | 1 |
| 1-178 | A1 | B178 | 2 | 1 |
| 1-179 | A1 | B179 | 2 | 1 |
| 1-180 | A1 | B180 | 2 | 1 |

TABLE 7

| Compound No. | Ln₁ | Ln₂ | n1 | n2 |
|---|---|---|---|---|
| 1-181 | A1 | B181 | 2 | 1 |
| 1-182 | A1 | B182 | 2 | 1 |
| 1-183 | A1 | B183 | 2 | 1 |
| 1-184 | A1 | B184 | 2 | 1 |
| 1-185 | A1 | B185 | 2 | 1 |
| 1-186 | A1 | B186 | 2 | 1 |
| 1-187 | A1 | B187 | 2 | 1 |
| 1-188 | A1 | B188 | 2 | 1 |
| 1-189 | A1 | B189 | 2 | 1 |
| 1-190 | A1 | B190 | 2 | 1 |
| 1-191 | A1 | B191 | 2 | 1 |
| 1-192 | A1 | B192 | 2 | 1 |
| 1-193 | A1 | B193 | 2 | 1 |
| 1-194 | A1 | B194 | 2 | 1 |
| 1-195 | A1 | B195 | 2 | 1 |
| 1-196 | A1 | B196 | 2 | 1 |
| 1-197 | A1 | B197 | 2 | 1 |
| 1-198 | A1 | B198 | 2 | 1 |
| 1-199 | A1 | B199 | 2 | 1 |
| 1-200 | A1 | B200 | 2 | 1 |
| 1-201 | A1 | B201 | 2 | 1 |
| 1-202 | A1 | B202 | 2 | 1 |
| 1-203 | A1 | B203 | 2 | 1 |
| 1-204 | A1 | B204 | 2 | 1 |
| 1-205 | A1 | B205 | 2 | 1 |
| 1-206 | A1 | B206 | 2 | 1 |
| 1-207 | A1 | B207 | 2 | 1 |
| 1-208 | A1 | B208 | 2 | 1 |
| 1-209 | A1 | B209 | 2 | 1 |
| 1-210 | A1 | B210 | 2 | 1 |

TABLE 8

| Compound No. | Ln₁ | Ln₂ | n1 | n2 |
|---|---|---|---|---|
| 1-211 | A1 | B211 | 2 | 1 |
| 1-212 | A1 | B212 | 2 | 1 |
| 1-213 | A1 | B213 | 2 | 1 |
| 1-214 | A1 | B214 | 2 | 1 |
| 1-215 | A1 | B215 | 2 | 1 |
| 1-216 | A1 | B216 | 2 | 1 |
| 1-217 | A1 | B217 | 2 | 1 |
| 1-218 | A1 | B218 | 2 | 1 |
| 1-219 | A1 | B219 | 2 | 1 |
| 1-220 | A1 | B220 | 2 | 1 |
| 1-221 | A1 | B221 | 2 | 1 |
| 1-222 | A1 | B222 | 2 | 1 |
| 1-223 | A1 | B223 | 2 | 1 |
| 1-224 | A1 | B224 | 2 | 1 |
| 1-225 | A1 | B225 | 2 | 1 |
| 1-226 | A1 | B226 | 2 | 1 |
| 1-227 | A1 | B227 | 2 | 1 |
| 1-228 | A1 | B228 | 2 | 1 |
| 1-229 | A1 | B229 | 2 | 1 |
| 1-230 | A1 | B230 | 2 | 1 |
| 1-231 | A1 | B231 | 2 | 1 |
| 1-232 | A1 | B232 | 2 | 1 |
| 1-233 | A1 | B233 | 2 | 1 |
| 1-234 | A1 | B234 | 2 | 1 |
| 1-235 | A1 | B235 | 2 | 1 |
| 1-236 | A1 | B236 | 2 | 1 |
| 1-237 | A1 | B237 | 2 | 1 |
| 1-238 | A1 | B238 | 2 | 1 |
| 1-239 | A1 | B239 | 2 | 1 |
| 1-240 | A1 | B240 | 2 | 1 |

TABLE 9

| Compound No. | Ln₁ | Ln₂ | n1 | n2 |
|---|---|---|---|---|
| 1-241 | A1 | B241 | 2 | 1 |
| 1-242 | A1 | B242 | 2 | 1 |
| 1-243 | A1 | B243 | 2 | 1 |
| 1-244 | A1 | B244 | 2 | 1 |
| 1-245 | A1 | B245 | 2 | 1 |
| 1-246 | A1 | B246 | 2 | 1 |
| 1-247 | A1 | B247 | 2 | 1 |
| 1-248 | A1 | B248 | 2 | 1 |
| 1-249 | A1 | B249 | 2 | 1 |
| 1-250 | A1 | B250 | 2 | 1 |
| 1-251 | A1 | B251 | 2 | 1 |
| 1-252 | A1 | B252 | 2 | 1 |
| 1-253 | A1 | B253 | 2 | 1 |
| 1-254 | A1 | B254 | 2 | 1 |
| 1-255 | A1 | B255 | 2 | 1 |
| 1-256 | A1 | B256 | 2 | 1 |
| 1-257 | A1 | B257 | 2 | 1 |
| 1-258 | A1 | B258 | 2 | 1 |
| 1-259 | A1 | B259 | 2 | 1 |
| 1-260 | A1 | B260 | 2 | 1 |
| 1-261 | A1 | B261 | 2 | 1 |
| 1-262 | A1 | B262 | 2 | 1 |
| 1-263 | A1 | B263 | 2 | 1 |
| 1-264 | A1 | B264 | 2 | 1 |
| 1-265 | A1 | B265 | 2 | 1 |
| 1-266 | A1 | B266 | 2 | 1 |
| 1-267 | A1 | B267 | 2 | 1 |
| 1-268 | A1 | B268 | 2 | 1 |
| 1-269 | A1 | B269 | 2 | 1 |
| 1-270 | A1 | B270 | 2 | 1 |

TABLE 10

| Compound No. | Ln₁ | Ln₂ | n1 | n2 |
|---|---|---|---|---|
| 1-271 | A1 | B271 | 2 | 1 |
| 1-272 | A1 | B272 | 2 | 1 |
| 1-273 | A1 | B273 | 2 | 1 |
| 1-274 | A1 | B274 | 2 | 1 |
| 1-275 | A1 | B275 | 2 | 1 |
| 1-276 | A1 | B276 | 2 | 1 |
| 1-277 | A1 | B277 | 2 | 1 |
| 1-278 | A1 | B278 | 2 | 1 |
| 1-279 | A1 | B279 | 2 | 1 |
| 1-280 | A1 | B280 | 2 | 1 |

TABLE 11

| Compound No. | Ln₁ | Ln₂ | n1 | n2 |
|---|---|---|---|---|
| 2-1 | A2 | B1 | 2 | 1 |
| 4-1 | A4 | B1 | 2 | 1 |
| 6-1 | A6 | B1 | 2 | 1 |
| 8-1 | A8 | B1 | 2 | 1 |
| 10-1 | A10 | B1 | 2 | 1 |
| 12-1 | A12 | B1 | 2 | 1 |
| 14-1 | A14 | B1 | 2 | 1 |
| 16-1 | A16 | B1 | 2 | 1 |
| 18-1 | A18 | B1 | 2 | 1 |
| 20-1 | A20 | B1 | 2 | 1 |
| 22-1 | A22 | B1 | 2 | 1 |
| 24-1 | A24 | B1 | 2 | 1 |
| 26-1 | A26 | B1 | 2 | 1 |
| 28-1 | A28 | B1 | 2 | 1 |
| 30-1 | A30 | B1 | 2 | 1 |
| 32-1 | A32 | B1 | 2 | 1 |
| 34-1 | A34 | B1 | 2 | 1 |
| 36-1 | A36 | B1 | 2 | 1 |
| 38-1 | A38 | B1 | 2 | 1 |
| 40-1 | A40 | B1 | 2 | 1 |
| 42-1 | A42 | B1 | 2 | 1 |
| 44-1 | A44 | B1 | 2 | 1 |
| 46-1 | A46 | B1 | 2 | 1 |
| 48-1 | A48 | B1 | 2 | 1 |
| 50-1 | A50 | B1 | 2 | 1 |
| 52-1 | A52 | B1 | 2 | 1 |
| 54-1 | A54 | B1 | 2 | 1 |
| 56-1 | A56 | B1 | 2 | 1 |
| 58-1 | A58 | B1 | 2 | 1 |
| 60-1 | A60 | B1 | 2 | 1 |

TABLE 12

| Compound No. | Ln₁ | Ln₂ | n1 | n2 |
|---|---|---|---|---|
| 62-1 | A62 | B1 | 2 | 1 |
| 64-1 | A64 | B1 | 2 | 1 |
| 66-1 | A66 | B1 | 2 | 1 |
| 68-1 | A68 | B1 | 2 | 1 |
| 70-1 | A70 | B1 | 2 | 1 |
| 73-1 | A73 | B1 | 2 | 1 |
| 76-1 | A76 | B1 | 2 | 1 |
| 80-1 | A80 | B1 | 2 | 1 |
| 84-1 | A84 | B1 | 2 | 1 |
| 87-1 | A87 | B1 | 2 | 1 |
| 90-1 | A90 | B1 | 2 | 1 |
| 93-1 | A93 | B1 | 2 | 1 |
| 96-1 | A96 | B1 | 2 | 1 |
| 99-1 | A99 | B1 | 2 | 1 |
| 102-1 | A102 | B1 | 2 | 1 |
| 105-1 | A105 | B1 | 2 | 1 |
| 108-1 | A108 | B1 | 2 | 1 |
| 120-1 | A120 | B1 | 2 | 1 |
| 125-1 | A125 | B1 | 2 | 1 |
| 129-1 | A129 | B1 | 2 | 1 |
| 130-1 | A130 | B1 | 2 | 1 |
| 133-1 | A133 | B1 | 2 | 1 |
| 137-1 | A137 | B1 | 2 | 1 |
| 140-1 | A140 | B1 | 2 | 1 |
| 143-1 | A143 | B1 | 2 | 1 |
| 146-1 | A146 | B1 | 2 | 1 |
| 149-1 | A149 | B1 | 2 | 1 |
| 151-1 | A151 | B1 | 2 | 1 |
| 152-1 | A152 | B1 | 2 | 1 |
| 153-1 | A153 | B1 | 2 | 1 |

TABLE 13

| Compound No. | Ln₁ | Ln₂ | n1 | n2 |
|---|---|---|---|---|
| 154-1 | A154 | B1 | 2 | 1 |
| 155-1 | A155 | B1 | 2 | 1 |
| 156-1 | A156 | B1 | 2 | 1 |
| 157-1 | A157 | B1 | 2 | 1 |
| 158-1 | A158 | B1 | 2 | 1 |
| 159-1 | A159 | B1 | 2 | 1 |
| 160-1 | A160 | B1 | 2 | 1 |
| 161-1 | A161 | B1 | 2 | 1 |
| 162-1 | A162 | B1 | 2 | 1 |
| 163-1 | A163 | B1 | 2 | 1 |
| 164-1 | A164 | B1 | 2 | 1 |
| 165-1 | A165 | B1 | 2 | 1 |
| 166-1 | A166 | B1 | 2 | 1 |
| 167-1 | A167 | B1 | 2 | 1 |
| 168-1 | A168 | B1 | 2 | 1 |
| 169-1 | A169 | B1 | 2 | 1 |
| 170-1 | A170 | B1 | 2 | 1 |
| 171-1 | A171 | B1 | 2 | 1 |
| 172-1 | A172 | B1 | 2 | 1 |
| 173-1 | A173 | B1 | 2 | 1 |
| 174-1 | A174 | B1 | 2 | 1 |
| 175-1 | A175 | B1 | 2 | 1 |
| 176-1 | A176 | B1 | 2 | 1 |
| 177-1 | A177 | B1 | 2 | 1 |
| 178-1 | A178 | B1 | 2 | 1 |
| 179-1 | A179 | B1 | 2 | 1 |
| 180-1 | A180 | B1 | 2 | 1 |
| 181-1 | A181 | B1 | 2 | 1 |
| 182-1 | A182 | B1 | 2 | 1 |
| 183-1 | A183 | B1 | 2 | 1 |

TABLE 14

| Compound No. | Ln₁ | Ln₂ | n1 | n2 |
|---|---|---|---|---|
| 184-1 | A184 | B1 | 2 | 1 |
| 185-1 | A185 | B1 | 2 | 1 |
| 186-1 | A186 | B1 | 2 | 1 |
| 187-1 | A187 | B1 | 2 | 1 |
| 188-1 | A188 | B1 | 2 | 1 |
| 189-1 | A189 | B1 | 2 | 1 |
| 190-1 | A190 | B1 | 2 | 1 |
| 191-1 | A191 | B1 | 2 | 1 |
| 192-1 | A192 | B1 | 2 | 1 |
| 193-1 | A193 | B1 | 2 | 1 |
| 194-1 | A194 | B1 | 2 | 1 |
| 195-1 | A195 | B1 | 2 | 1 |
| 196-1 | A196 | B1 | 2 | 1 |
| 197-1 | A197 | B1 | 2 | 1 |
| 198-1 | A198 | B1 | 2 | 1 |
| 199-1 | A199 | B1 | 2 | 1 |
| 200-1 | A200 | B1 | 2 | 1 |
| 201-1 | A201 | B1 | 2 | 1 |
| 202-1 | A202 | B1 | 2 | 1 |
| 203-1 | A203 | B1 | 2 | 1 |
| 204-1 | A204 | B1 | 2 | 1 |
| 205-1 | A205 | B1 | 2 | 1 |
| 206-1 | A206 | B1 | 2 | 1 |
| 207-1 | A207 | B1 | 2 | 1 |
| 208-1 | A208 | B1 | 2 | 1 |
| 209-1 | A209 | B1 | 2 | 1 |
| 210-1 | A210 | B1 | 2 | 1 |
| 211-1 | A211 | B1 | 2 | 1 |
| 212-1 | A212 | B1 | 2 | 1 |
| 213-1 | A213 | B1 | 2 | 1 |

TABLE 15

| Compound No. | Ln₁ | Ln₂ | n1 | n2 |
|---|---|---|---|---|
| 214-1 | A214 | B1 | 2 | 1 |
| 215-1 | A215 | B1 | 2 | 1 |
| 216-1 | A216 | B1 | 2 | 1 |
| 217-1 | A217 | B1 | 2 | 1 |
| 218-1 | A218 | B1 | 2 | 1 |
| 219-1 | A219 | B1 | 2 | 1 |
| 220-1 | A220 | B1 | 2 | 1 |
| 221-1 | A221 | B1 | 2 | 1 |
| 222-1 | A222 | B1 | 2 | 1 |
| 223-1 | A223 | B1 | 2 | 1 |
| 224-1 | A224 | B1 | 2 | 1 |
| 225-1 | A225 | B1 | 2 | 1 |
| 226-1 | A226 | B1 | 2 | 1 |
| 227-1 | A227 | B1 | 2 | 1 |
| 228-1 | A228 | B1 | 2 | 1 |
| 229-1 | A229 | B1 | 2 | 1 |
| 230-1 | A230 | B1 | 2 | 1 |
| 231-1 | A231 | B1 | 2 | 1 |
| 232-1 | A232 | B1 | 2 | 1 |
| 233-1 | A233 | B1 | 2 | 1 |
| 234-1 | A234 | B1 | 2 | 1 |
| 235-1 | A235 | B1 | 2 | 1 |
| 236-1 | A236 | B1 | 2 | 1 |
| 237-1 | A237 | B1 | 2 | 1 |
| 238-1 | A238 | B1 | 2 | 1 |
| 239-1 | A239 | B1 | 2 | 1 |
| 240-1 | A240 | B1 | 2 | 1 |
| 241-1 | A241 | B1 | 2 | 1 |
| 242-1 | A242 | B1 | 2 | 1 |
| 243-1 | A243 | B1 | 2 | 1 |

TABLE 16

| Compound No. | Ln₁ | Ln₂ | n1 | n2 |
|---|---|---|---|---|
| 244-1 | A244 | B1 | 2 | 1 |
| 245-1 | A245 | B1 | 2 | 1 |
| 246-1 | A246 | B1 | 2 | 1 |
| 247-1 | A247 | B1 | 2 | 1 |
| 248-1 | A248 | B1 | 2 | 1 |
| 249-1 | A249 | B1 | 2 | 1 |
| 250-1 | A250 | B1 | 2 | 1 |
| 251-1 | A251 | B1 | 2 | 1 |
| 252-1 | A252 | B1 | 2 | 1 |
| 253-1 | A253 | B1 | 2 | 1 |
| 254-1 | A254 | B1 | 2 | 1 |
| 255-1 | A255 | B1 | 2 | 1 |
| 256-1 | A256 | B1 | 2 | 1 |
| 257-1 | A257 | B1 | 2 | 1 |
| 258-1 | A258 | B1 | 2 | 1 |
| 259-1 | A259 | B1 | 2 | 1 |
| 260-1 | A260 | B1 | 2 | 1 |
| 261-1 | A261 | B1 | 2 | 1 |
| 262-1 | A262 | B1 | 2 | 1 |
| 263-1 | A263 | B1 | 2 | 1 |
| 264-1 | A264 | B1 | 2 | 1 |
| 265-1 | A265 | B1 | 2 | 1 |
| 266-1 | A266 | B1 | 2 | 1 |
| 267-1 | A267 | B1 | 2 | 1 |
| 268-1 | A268 | B1 | 2 | 1 |
| 269-1 | A269 | B1 | 2 | 1 |
| 270-1 | A270 | B1 | 2 | 1 |
| 271-1 | A271 | B1 | 2 | 1 |
| 272-1 | A272 | B1 | 2 | 1 |
| 273-1 | A273 | B1 | 2 | 1 |

TABLE 17

| Compound No. | Ln₁ | Ln₂ | n1 | n2 |
|---|---|---|---|---|
| 274-1 | A274 | B1 | 2 | 1 |
| 275-1 | A275 | B1 | 2 | 1 |
| 276-1 | A276 | B1 | 2 | 1 |
| 277-1 | A277 | B1 | 2 | 1 |
| 278-1 | A278 | B1 | 2 | 1 |
| 279-1 | A279 | B1 | 2 | 1 |
| 280-1 | A280 | B1 | 2 | 1 |
| 281-1 | A281 | B1 | 2 | 1 |
| 282-1 | A282 | B1 | 2 | 1 |
| 283-1 | A283 | B1 | 2 | 1 |
| 284-1 | A284 | B1 | 2 | 1 |
| 285-1 | A285 | B1 | 2 | 1 |
| 2-4 | A2 | B4 | 2 | 1 |
| 2-8 | A2 | B8 | 2 | 1 |
| 2-10 | A2 | B10 | 2 | 1 |
| 2-13 | A2 | B13 | 2 | 1 |
| 2-20 | A2 | B20 | 2 | 1 |
| 2-24 | A2 | B24 | 2 | 1 |
| 2-27 | A2 | B27 | 2 | 1 |
| 2-48 | A2 | B48 | 2 | 1 |
| 2-62 | A2 | B62 | 2 | 1 |
| 2-63 | A2 | B63 | 2 | 1 |
| 2-85 | A2 | B85 | 2 | 1 |
| 2-91 | A2 | B91 | 2 | 1 |
| 2-108 | A2 | B108 | 2 | 1 |
| 2-116 | A2 | B116 | 2 | 1 |
| 2-137 | A2 | B137 | 2 | 1 |
| 2-144 | A2 | B144 | 2 | 1 |
| 2-152 | A2 | B152 | 2 | 1 |

TABLE 18

| Compound No. | Ln₁ | Ln₂ | n1 | n2 |
|---|---|---|---|---|
| 2-116 | A2 | B116 | 2 | 1 |
| 2-137 | A2 | B137 | 2 | 1 |
| 2-144 | A2 | B144 | 2 | 1 |
| 2-152 | A2 | B152 | 2 | 1 |
| 2-162 | A2 | B162 | 2 | 1 |
| 2-169 | A2 | B169 | 2 | 1 |
| 2-176 | A2 | B176 | 2 | 1 |
| 2-187 | A2 | B187 | 2 | 1 |
| 2-201 | A2 | B201 | 2 | 1 |
| 2-213 | A2 | B213 | 2 | 1 |
| 2-249 | A2 | B249 | 2 | 1 |
| 2-252 | A2 | B252 | 2 | 1 |
| 2-255 | A2 | B255 | 2 | 1 |
| 2-265 | A2 | B265 | 2 | 1 |
| 2-273 | A2 | B273 | 2 | 1 |
| 2-277 | A2 | B277 | 2 | 1 |
| 3-4 | A3 | B4 | 2 | 1 |
| 4-8 | A4 | B8 | 2 | 1 |
| 5-10 | A5 | B10 | 2 | 1 |
| 6-13 | A6 | B13 | 2 | 1 |
| 7-20 | A7 | B20 | 2 | 1 |
| 8-24 | A8 | B24 | 2 | 1 |
| 9-27 | A9 | B27 | 2 | 1 |
| 10-48 | A10 | B48 | 2 | 1 |
| 12-63 | A12 | B63 | 2 | 1 |
| 13-85 | A13 | B85 | 2 | 1 |
| 14-91 | A14 | B91 | 2 | 1 |
| 15-108 | A15 | B108 | 2 | 1 |
| 16-116 | A16 | B116 | 2 | 1 |
| 17-137 | A17 | B137 | 2 | 1 |

TABLE 19

| Compound No. | Ln₁ | Ln₂ | n1 | n2 |
|---|---|---|---|---|
| 18-141 | A18 | B138 | 2 | 1 |
| 19-142 | A19 | B139 | 2 | 1 |
| 20-143 | A20 | B140 | 2 | 1 |
| 21-144 | A21 | B141 | 2 | 1 |
| 22-145 | A22 | B142 | 2 | 1 |
| 23-146 | A23 | B143 | 2 | 1 |
| 24-147 | A24 | B144 | 2 | 1 |
| 25-148 | A25 | B145 | 2 | 1 |
| 26-149 | A26 | B146 | 2 | 1 |
| 27-150 | A27 | B147 | 2 | 1 |
| 28-151 | A28 | B148 | 2 | 1 |
| 29-152 | A29 | B149 | 2 | 1 |
| 30-153 | A30 | B150 | 2 | 1 |
| 31-154 | A31 | B151 | 2 | 1 |
| 32-155 | A32 | B152 | 2 | 1 |
| 33-156 | A33 | B153 | 2 | 1 |
| 34-157 | A34 | B154 | 2 | 1 |
| 35-158 | A35 | B155 | 2 | 1 |
| 36-159 | A36 | B156 | 2 | 1 |
| 37-160 | A37 | B157 | 2 | 1 |
| 38-161 | A38 | B158 | 2 | 1 |
| 39-162 | A39 | B159 | 2 | 1 |
| 40-163 | A40 | B160 | 2 | 1 |
| 41-164 | A41 | B161 | 2 | 1 |
| 42-165 | A42 | B162 | 2 | 1 |
| 43-166 | A43 | B163 | 2 | 1 |
| 44-167 | A44 | B164 | 2 | 1 |
| 45-141 | A45 | B165 | 2 | 1 |
| 46-142 | A46 | B166 | 2 | 1 |
| 47-143 | A47 | B167 | 2 | 1 |

TABLE 20

| Compound No. | Ln₁ | Ln₂ | n1 | n2 |
|---|---|---|---|---|
| 48-168 | A48 | B168 | 2 | 1 |
| 49-169 | A49 | B169 | 2 | 1 |
| 50-170 | A50 | B170 | 2 | 1 |
| 51-171 | A51 | B171 | 2 | 1 |
| 52-172 | A52 | B172 | 2 | 1 |
| 53-173 | A53 | B173 | 2 | 1 |
| 54-174 | A54 | B174 | 2 | 1 |
| 55-175 | A55 | B175 | 2 | 1 |
| 56-176 | A56 | B176 | 2 | 1 |
| 57-177 | A57 | B177 | 2 | 1 |
| 58-178 | A58 | B178 | 2 | 1 |
| 59-179 | A59 | B179 | 2 | 1 |
| 60-180 | A60 | B180 | 2 | 1 |
| 61-181 | A61 | B181 | 2 | 1 |
| 62-182 | A62 | B182 | 2 | 1 |
| 63-183 | A63 | B183 | 2 | 1 |
| 64-184 | A64 | B184 | 2 | 1 |
| 65-185 | A65 | B185 | 2 | 1 |
| 66-186 | A66 | B186 | 2 | 1 |
| 67-187 | A67 | B187 | 2 | 1 |
| 68-188 | A68 | B188 | 2 | 1 |
| 69-189 | A69 | B189 | 2 | 1 |
| 70-190 | A70 | B190 | 2 | 1 |
| 71-191 | A71 | B191 | 2 | 1 |
| 72-192 | A72 | B192 | 2 | 1 |
| 73-193 | A73 | B193 | 2 | 1 |
| 74-194 | A74 | B194 | 2 | 1 |
| 75-195 | A75 | B195 | 2 | 1 |
| 76-196 | A76 | B196 | 2 | 1 |
| 77-197 | A77 | B197 | 2 | 1 |

TABLE 21

| Compound No. | Ln₁ | Ln₂ | n1 | n2 |
|---|---|---|---|---|
| 78-198 | A78 | B198 | 2 | 1 |
| 79-199 | A79 | B199 | 2 | 1 |
| 80-200 | A80 | B200 | 2 | 1 |
| 81-201 | A81 | B201 | 2 | 1 |
| 82-202 | A82 | B202 | 2 | 1 |
| 83-203 | A83 | B203 | 2 | 1 |
| 84-204 | A84 | B204 | 2 | 1 |
| 85-205 | A85 | B205 | 2 | 1 |
| 86-206 | A86 | B206 | 2 | 1 |
| 87-207 | A87 | B207 | 2 | 1 |
| 88-208 | A88 | B208 | 2 | 1 |
| 89-209 | A89 | B209 | 2 | 1 |
| 90-210 | A90 | B210 | 2 | 1 |
| 91-211 | A91 | B211 | 2 | 1 |
| 92-212 | A92 | B212 | 2 | 1 |
| 93-213 | A93 | B213 | 2 | 1 |
| 94-214 | A94 | B214 | 2 | 1 |
| 95-215 | A95 | B215 | 2 | 1 |
| 96-216 | A96 | B216 | 2 | 1 |
| 97-217 | A97 | B217 | 2 | 1 |
| 98-218 | A98 | B218 | 2 | 1 |
| 99-219 | A99 | B219 | 2 | 1 |
| 100-220 | A100 | B220 | 2 | 1 |
| 151-221 | A151 | B221 | 2 | 1 |
| 152-222 | A152 | B222 | 2 | 1 |
| 153-223 | A153 | B223 | 2 | 1 |
| 154-224 | A154 | B224 | 2 | 1 |
| 155-225 | A155 | B225 | 2 | 1 |
| 156-226 | A156 | B226 | 2 | 1 |
| 157-227 | A157 | B227 | 2 | 1 |

TABLE 22

| Compound No. | Ln₁ | Ln₂ | n1 | n2 |
|---|---|---|---|---|
| 158-228 | A158 | B228 | 2 | 1 |
| 159-229 | A159 | B229 | 2 | 1 |
| 160-230 | A160 | B230 | 2 | 1 |
| 161-231 | A161 | B231 | 2 | 1 |
| 162-232 | A162 | B232 | 2 | 1 |
| 163-233 | A163 | B233 | 2 | 1 |
| 164-234 | A164 | B234 | 2 | 1 |
| 165-235 | A165 | B235 | 2 | 1 |
| 166-236 | A166 | B236 | 2 | 1 |
| 167-237 | A167 | B237 | 2 | 1 |
| 168-238 | A168 | B238 | 2 | 1 |
| 169-239 | A169 | B239 | 2 | 1 |
| 170-240 | A170 | B240 | 2 | 1 |
| 171-241 | A171 | B241 | 2 | 1 |
| 172-242 | A172 | B242 | 2 | 1 |
| 173-243 | A173 | B243 | 2 | 1 |
| 174-244 | A174 | B244 | 2 | 1 |
| 175-245 | A175 | B245 | 2 | 1 |
| 176-246 | A176 | B246 | 2 | 1 |
| 177-247 | A177 | B247 | 2 | 1 |
| 178-248 | A178 | B248 | 2 | 1 |
| 179-249 | A179 | B249 | 2 | 1 |
| 180-250 | A180 | B250 | 2 | 1 |
| 181-251 | A181 | B251 | 2 | 1 |
| 182-252 | A182 | B252 | 2 | 1 |
| 183-253 | A183 | B253 | 2 | 1 |
| 184-254 | A184 | B254 | 2 | 1 |
| 185-255 | A185 | B255 | 2 | 1 |
| 186-256 | A186 | B256 | 2 | 1 |
| 187-257 | A187 | B257 | 2 | 1 |

TABLE 23

| Compound No. | Ln₁ | Ln₂ | n1 | n2 |
|---|---|---|---|---|
| 188-258 | A138 | B258 | 2 | 1 |
| 189-259 | A139 | B259 | 2 | 1 |
| 190-260 | A140 | B260 | 2 | 1 |
| 191-261 | A141 | B261 | 2 | 1 |
| 192-262 | A142 | B262 | 2 | 1 |
| 193-263 | A143 | B263 | 2 | 1 |
| 194-264 | A144 | B264 | 2 | 1 |
| 195-265 | A145 | B265 | 2 | 1 |
| 196-266 | A146 | B266 | 2 | 1 |
| 197-267 | A147 | B267 | 2 | 1 |
| 198-268 | A148 | B268 | 2 | 1 |
| 199-269 | A149 | B269 | 2 | 1 |
| 200-270 | A150 | B270 | 2 | 1 |
| 201-271 | A151 | B271 | 2 | 1 |
| 202-272 | A152 | B272 | 2 | 1 |
| 203-273 | A153 | B273 | 2 | 1 |
| 204-274 | A154 | B274 | 2 | 1 |
| 205-275 | A155 | B275 | 2 | 1 |
| 206-276 | A156 | B276 | 2 | 1 |
| 207-277 | A157 | B277 | 2 | 1 |
| 208-278 | A158 | B278 | 2 | 1 |
| 209-279 | A159 | B279 | 2 | 1 |
| 210-280 | A160 | B280 | 2 | 1 |
| 23-14 | A23 | B14 | 2 | 1 |
| 35-50 | A35 | B50 | 2 | 1 |
| 46-71 | A46 | B71 | 2 | 1 |
| 58-92 | A58 | B92 | 2 | 1 |
| 66-103 | A66 | B103 | 2 | 1 |
| 75-116 | A75 | B116 | 2 | 1 |
| 87-178 | A87 | B178 | 2 | 1 |

TABLE 24

| Compound No. | Ln₁ | Ln₂ | n1 | n2 |
|---|---|---|---|---|
| 101-1 | A101 | B1 | 2 | 1 |
| 102-2 | A102 | B2 | 2 | 1 |
| 103-3 | A103 | B3 | 2 | 1 |
| 104-4 | A104 | B4 | 2 | 1 |
| 105-5 | A105 | B5 | 2 | 1 |
| 106-6 | A106 | B6 | 2 | 1 |
| 107-7 | A107 | B7 | 2 | 1 |
| 108-8 | A108 | B8 | 2 | 1 |
| 109-9 | A109 | B9 | 2 | 1 |
| 110-10 | A110 | B10 | 2 | 1 |
| 111-11 | A111 | B11 | 2 | 1 |
| 112-12 | A112 | B12 | 2 | 1 |
| 113-13 | A113 | B13 | 2 | 1 |
| 114-14 | A114 | B14 | 2 | 1 |
| 115-49 | A115 | B49 | 2 | 1 |
| 116-16 | A116 | B16 | 2 | 1 |
| 117-17 | A117 | B17 | 2 | 1 |
| 118-18 | A118 | B18 | 2 | 1 |
| 119-19 | A119 | B19 | 2 | 1 |
| 120-20 | A120 | B20 | 2 | 1 |
| 121-21 | A121 | B21 | 2 | 1 |
| 122-22 | A122 | B22 | 2 | 1 |
| 123-23 | A123 | B23 | 2 | 1 |
| 124-24 | A124 | B24 | 2 | 1 |
| 125-25 | A125 | B25 | 2 | 1 |
| 126-26 | A126 | B26 | 2 | 1 |
| 127-27 | A127 | B27 | 2 | 1 |
| 128-28 | A128 | B28 | 2 | 1 |
| 129-29 | A129 | B29 | 2 | 1 |
| 130-30 | A130 | B30 | 2 | 1 |

TABLE 25

| Compound No. | Ln₁ | Ln₂ | n1 | n2 |
|---|---|---|---|---|
| 131-31 | A131 | B31 | 2 | 1 |
| 132-32 | A132 | B32 | 2 | 1 |
| 133-33 | A133 | B33 | 2 | 1 |
| 134-34 | A134 | B34 | 2 | 1 |
| 135-35 | A135 | B35 | 2 | 1 |
| 136-36 | A136 | B36 | 2 | 1 |
| 137-37 | A137 | B37 | 2 | 1 |
| 138-38 | A138 | B38 | 2 | 1 |
| 139-39 | A139 | B39 | 2 | 1 |
| 140-40 | A140 | B40 | 2 | 1 |
| 141-41 | A141 | B41 | 2 | 1 |
| 142-42 | A142 | B42 | 2 | 1 |
| 143-43 | A143 | B43 | 2 | 1 |
| 144-44 | A144 | B44 | 2 | 1 |
| 145-45 | A145 | B45 | 2 | 1 |
| 146-46 | A146 | B46 | 2 | 1 |
| 147-47 | A147 | B47 | 2 | 1 |
| 148-53 | A148 | B53 | 2 | 1 |
| 149-15 | A149 | B15 | 2 | 1 |
| 150-50 | A150 | B50 | 2 | 1 |
| 241-51 | A241 | B51 | 2 | 1 |
| 242-52 | A242 | B52 | 2 | 1 |
| 243-48 | A243 | B48 | 2 | 1 |
| 244-54 | A244 | B54 | 2 | 1 |
| 245-55 | A245 | B55 | 2 | 1 |
| 246-56 | A246 | B56 | 2 | 1 |
| 247-57 | A247 | B57 | 2 | 1 |
| 248-58 | A248 | B58 | 2 | 1 |
| 249-59 | A249 | B59 | 2 | 1 |
| 250-60 | A250 | B60 | 2 | 1 |

TABLE 26

| Compound No. | Ln₁ | Ln₂ | n1 | n2 |
|---|---|---|---|---|
| 251-61 | A251 | B61 | 2 | 1 |
| 252-62 | A252 | B62 | 2 | 1 |
| 253-63 | A253 | B63 | 2 | 1 |
| 254-64 | A254 | B64 | 2 | 1 |
| 255-65 | A255 | B65 | 2 | 1 |
| 256-66 | A256 | B66 | 2 | 1 |
| 257-67 | A257 | B67 | 2 | 1 |
| 258-68 | A258 | B68 | 2 | 1 |
| 259-69 | A259 | B69 | 2 | 1 |
| 260-70 | A260 | B70 | 2 | 1 |
| 261-71 | A261 | B71 | 2 | 1 |
| 262-72 | A262 | B72 | 2 | 1 |
| 263-73 | A263 | B73 | 2 | 1 |
| 264-74 | A264 | B74 | 2 | 1 |
| 265-75 | A265 | B75 | 2 | 1 |
| 266-76 | A266 | B76 | 2 | 1 |
| 267-77 | A267 | B77 | 2 | 1 |
| 268-78 | A268 | B78 | 2 | 1 |
| 269-79 | A269 | B79 | 2 | 1 |
| 270-80 | A270 | B80 | 2 | 1 |
| 271-81 | A271 | B81 | 2 | 1 |
| 272-82 | A272 | B82 | 2 | 1 |
| 273-83 | A273 | B83 | 2 | 1 |
| 274-84 | A274 | B84 | 2 | 1 |
| 275-85 | A275 | B85 | 2 | 1 |
| 276-86 | A276 | B86 | 2 | 1 |
| 277-87 | A277 | B87 | 2 | 1 |
| 278-88 | A278 | B88 | 2 | 1 |
| 279-89 | A279 | B89 | 2 | 1 |
| 280-90 | A280 | B90 | 2 | 1 |

TABLE 27

| Compound No. | Ln₁ | Ln₂ | n1 | n2 |
|---|---|---|---|---|
| 281-91 | A281 | B91 | 2 | 1 |
| 281-92 | A282 | B92 | 2 | 1 |
| 281-93 | A283 | B93 | 2 | 1 |
| 135-281 | A135 | B281 | 2 | 1 |
| 135-282 | A135 | B282 | 2 | 1 |
| 135-283 | A135 | B283 | 2 | 1 |
| 135-284 | A135 | B284 | 2 | 1 |
| 135-285 | A135 | B285 | 2 | 1 |
| 135-286 | A135 | B286 | 2 | 1 |
| 135-287 | A135 | B287 | 2 | 1 |
| 135-288 | A135 | B288 | 2 | 1 |
| 135-289 | A135 | B289 | 2 | 1 |
| 135-290 | A135 | B290 | 2 | 1 |
| 135-291 | A135 | B291 | 2 | 1 |
| 135-292 | A135 | B292 | 2 | 1 |
| 135-293 | A135 | B293 | 2 | 1 |
| 135-294 | A135 | B294 | 2 | 1 |
| 135-295 | A135 | B295 | 2 | 1 |
| 135-296 | A135 | B296 | 2 | 1 |
| 135-297 | A135 | B297 | 2 | 1 |
| 135-298 | A135 | B298 | 2 | 1 |
| 135-299 | A135 | B299 | 2 | 1 |
| 135-300 | A135 | B300 | 2 | 1 |
| 135-301 | A135 | B301 | 2 | 1 |
| 135-302 | A135 | B302 | 2 | 1 |
| 135-303 | A135 | B303 | 2 | 1 |
| 135-304 | A135 | B304 | 2 | 1 |
| 135-305 | A135 | B305 | 2 | 1 |
| 135-306 | A135 | B306 | 2 | 1 |
| 135-307 | A135 | B307 | 2 | 1 |

TABLE 28

| Compound No. | Ln₁ | Ln₂ | n1 | n2 |
|---|---|---|---|---|
| 145-308 | A145 | B308 | 2 | 1 |
| 145-309 | A145 | B309 | 2 | 1 |
| 145-310 | A145 | B310 | 2 | 1 |
| 145-311 | A145 | B311 | 2 | 1 |
| 145-312 | A145 | B312 | 2 | 1 |
| 145-313 | A145 | B313 | 2 | 1 |
| 145-314 | A145 | B314 | 2 | 1 |
| 145-315 | A145 | B315 | 2 | 1 |
| 145-316 | A145 | B316 | 2 | 1 |
| 145-317 | A145 | B317 | 2 | 1 |
| 145-318 | A145 | B318 | 2 | 1 |
| 145-319 | A145 | B319 | 2 | 1 |
| 145-320 | A145 | B320 | 2 | 1 |
| 145-321 | A145 | B321 | 2 | 1 |
| 145-322 | A145 | B322 | 2 | 1 |
| 145-323 | A145 | B323 | 2 | 1 |
| 145-324 | A145 | B324 | 2 | 1 |
| 145-325 | A145 | B325 | 2 | 1 |
| 145-326 | A145 | B326 | 2 | 1 |
| 145-327 | A145 | B327 | 2 | 1 |
| 145-328 | A145 | B328 | 2 | 1 |
| 145-329 | A145 | B329 | 2 | 1 |
| 145-330 | A145 | B330 | 2 | 1 |
| 145-331 | A145 | B331 | 2 | 1 |
| 145-332 | A145 | B332 | 2 | 1 |
| 145-333 | A145 | B333 | 2 | 1 |
| 145-334 | A145 | B334 | 2 | 1 |
| 145-335 | A145 | B335 | 2 | 1 |
| 145-336 | A145 | B336 | 2 | 1 |
| 145-337 | A145 | B337 | 2 | 1 |

TABLE 29

| Compound No. | Ln₁ | Ln₂ | n1 | n2 |
|---|---|---|---|---|
| 145-338 | A145 | B338 | 2 | 1 |
| 145-339 | A145 | B339 | 2 | 1 |
| 145-340 | A145 | B340 | 2 | 1 |
| 145-341 | A145 | B341 | 2 | 1 |
| 145-342 | A145 | B342 | 2 | 1 |
| 145-343 | A145 | B343 | 2 | 1 |
| 145-344 | A145 | B344 | 2 | 1 |
| 145-345 | A145 | B345 | 2 | 1 |
| 145-346 | A145 | B346 | 2 | 1 |
| 145-347 | A145 | B347 | 2 | 1 |
| 145-348 | A145 | B348 | 2 | 1 |
| 145-349 | A145 | B349 | 2 | 1 |
| 145-350 | A145 | B350 | 2 | 1 |
| 145-351 | A145 | B351 | 2 | 1 |
| 145-352 | A145 | B352 | 2 | 1 |
| 145-353 | A145 | B353 | 2 | 1 |
| 145-354 | A145 | B354 | 2 | 1 |
| 145-355 | A145 | B355 | 2 | 1 |
| 145-356 | A145 | B356 | 2 | 1 |
| 145-357 | A145 | B357 | 2 | 1 |
| 145-358 | A145 | B358 | 2 | 1 |
| 145-359 | A145 | B359 | 2 | 1 |
| 145-360 | A145 | B360 | 2 | 1 |
| 145-361 | A145 | B361 | 2 | 1 |
| 145-362 | A145 | B362 | 2 | 1 |
| 145-363 | A145 | B363 | 2 | 1 |
| 145-364 | A145 | B364 | 2 | 1 |
| 145-365 | A145 | B365 | 2 | 1 |
| 145-366 | A145 | B366 | 2 | 1 |
| 145-367 | A145 | B367 | 2 | 1 |

TABLE 30

| Compound No. | Ln₁ | Ln₂ | n1 | n2 |
|---|---|---|---|---|
| 145-368 | A145 | B368 | 2 | 1 |
| 145-369 | A145 | B369 | 2 | 1 |
| 145-370 | A145 | B370 | 2 | 1 |
| 145-371 | A145 | B371 | 2 | 1 |
| 145-372 | A145 | B372 | 2 | 1 |
| 145-373 | A145 | B373 | 2 | 1 |
| 145-374 | A145 | B374 | 2 | 1 |
| 145-375 | A145 | B375 | 2 | 1 |
| 145-376 | A145 | B376 | 2 | 1 |
| 145-377 | A145 | B377 | 2 | 1 |
| 145-378 | A145 | B378 | 2 | 1 |
| 145-379 | A145 | B379 | 2 | 1 |
| 145-380 | A145 | B380 | 2 | 1 |
| 145-381 | A145 | B381 | 2 | 1 |
| 145-382 | A145 | B382 | 2 | 1 |
| 145-383 | A145 | B383 | 2 | 1 |
| 145-384 | A145 | B384 | 2 | 1 |
| 145-385 | A145 | B385 | 2 | 1 |
| 145-386 | A145 | B386 | 2 | 1 |
| 145-387 | A145 | B387 | 2 | 1 |
| 145-388 | A145 | B388 | 2 | 1 |
| 145-389 | A145 | B389 | 2 | 1 |
| 145-390 | A145 | B390 | 2 | 1 |
| 145-391 | A145 | B391 | 2 | 1 |
| 145-392 | A145 | B392 | 2 | 1 |
| 145-393 | A145 | B393 | 2 | 1 |
| 145-394 | A145 | B394 | 2 | 1 |
| 145-395 | A145 | B395 | 2 | 1 |
| 145-396 | A145 | B396 | 2 | 1 |
| 145-397 | A145 | B397 | 2 | 1 |

TABLE 31

| Compound No. | Ln₁ | Ln₂ | n1 | n2 |
|---|---|---|---|---|
| 147-398 | A147 | B398 | 2 | 1 |
| 147-399 | A147 | B399 | 2 | 1 |
| 147-400 | A147 | B400 | 2 | 1 |
| 147-401 | A147 | B401 | 2 | 1 |
| 147-402 | A147 | B402 | 2 | 1 |
| 147-403 | A147 | B403 | 2 | 1 |
| 147-404 | A147 | B404 | 2 | 1 |
| 147-405 | A147 | B405 | 2 | 1 |
| 147-406 | A147 | B406 | 2 | 1 |
| 147-407 | A147 | B407 | 2 | 1 |
| 147-408 | A147 | B408 | 2 | 1 |
| 147-409 | A147 | B409 | 2 | 1 |
| 147-410 | A147 | B410 | 2 | 1 |
| 147-411 | A147 | B411 | 2 | 1 |
| 147-412 | A147 | B412 | 2 | 1 |
| 147-413 | A147 | B413 | 2 | 1 |
| 147-414 | A147 | B414 | 2 | 1 |
| 147-415 | A147 | B415 | 2 | 1 |
| 147-416 | A147 | B416 | 2 | 1 |
| 147-417 | A147 | B417 | 2 | 1 |
| 147-418 | A147 | B418 | 2 | 1 |
| 147-419 | A147 | B419 | 2 | 1 |
| 147-420 | A147 | B420 | 2 | 1 |
| 218-2 | A218 | B2 | 2 | 1 |
| 218-3 | A218 | B3 | 2 | 1 |
| 218-4 | A218 | B4 | 2 | 1 |
| 218-5 | A218 | B5 | 2 | 1 |
| 218-6 | A218 | B6 | 2 | 1 |
| 218-7 | A218 | B7 | 2 | 1 |
| 218-8 | A218 | B8 | 2 | 1 |

TABLE 32

| Compound No. | Ln₁ | Ln₂ | n1 | n2 |
|---|---|---|---|---|
| 218-9 | A218 | B9 | 2 | 1 |
| 218-10 | A218 | B10 | 2 | 1 |
| 218-11 | A218 | B11 | 2 | 1 |
| 218-12 | A218 | B12 | 2 | 1 |
| 218-13 | A218 | B13 | 2 | 1 |
| 218-14 | A218 | B14 | 2 | 1 |
| 218-15 | A218 | B15 | 2 | 1 |
| 218-16 | A218 | B16 | 2 | 1 |
| 218-17 | A218 | B17 | 2 | 1 |
| 218-18 | A218 | B18 | 2 | 1 |
| 218-19 | A218 | B19 | 2 | 1 |
| 218-20 | A218 | B20 | 2 | 1 |
| 218-21 | A218 | B21 | 2 | 1 |
| 218-22 | A218 | B22 | 2 | 1 |
| 218-23 | A218 | B23 | 2 | 1 |
| 218-24 | A218 | B24 | 2 | 1 |
| 218-25 | A218 | B25 | 2 | 1 |
| 218-26 | A218 | B26 | 2 | 1 |
| 218-27 | A218 | B27 | 2 | 1 |
| 218-28 | A218 | B28 | 2 | 1 |
| 218-29 | A218 | B29 | 2 | 1 |
| 218-30 | A218 | B30 | 2 | 1 |
| 218-31 | A218 | B31 | 2 | 1 |
| 218-32 | A218 | B32 | 2 | 1 |
| 218-33 | A218 | B33 | 2 | 1 |
| 218-34 | A218 | B34 | 2 | 1 |
| 218-35 | A218 | B35 | 2 | 1 |
| 218-36 | A218 | B36 | 2 | 1 |
| 218-37 | A218 | B37 | 2 | 1 |
| 218-38 | A218 | B38 | 2 | 1 |

TABLE 33

| Compound No. | Ln₁ | Ln₂ | n1 | n2 |
|---|---|---|---|---|
| 218-39 | A218 | B39 | 2 | 1 |
| 218-40 | A218 | B40 | 2 | 1 |
| 218-41 | A218 | B41 | 2 | 1 |
| 218-42 | A218 | B42 | 2 | 1 |
| 218-43 | A218 | B43 | 2 | 1 |
| 218-44 | A218 | B44 | 2 | 1 |
| 218-45 | A218 | B45 | 2 | 1 |
| 218-46 | A218 | B46 | 2 | 1 |
| 218-47 | A218 | B47 | 2 | 1 |
| 218-48 | A218 | B48 | 2 | 1 |
| 218-49 | A218 | B49 | 2 | 1 |
| 218-50 | A218 | B50 | 2 | 1 |
| 218-51 | A218 | B51 | 2 | 1 |
| 218-52 | A218 | B52 | 2 | 1 |
| 218-53 | A218 | B53 | 2 | 1 |
| 218-54 | A218 | B54 | 2 | 1 |
| 218-55 | A218 | B55 | 2 | 1 |
| 218-56 | A218 | B56 | 2 | 1 |
| 218-57 | A218 | B57 | 2 | 1 |
| 218-58 | A218 | B58 | 2 | 1 |
| 218-59 | A218 | B59 | 2 | 1 |
| 218-60 | A218 | B60 | 2 | 1 |
| 218-61 | A218 | B61 | 2 | 1 |
| 218-62 | A218 | B62 | 2 | 1 |
| 218-63 | A218 | B63 | 2 | 1 |
| 218-64 | A218 | B64 | 2 | 1 |
| 218-65 | A218 | B65 | 2 | 1 |
| 218-66 | A218 | B66 | 2 | 1 |
| 218-67 | A218 | B67 | 2 | 1 |
| 218-68 | A218 | B68 | 2 | 1 |

TABLE 34

| Compound No. | Ln₁ | Ln₂ | n1 | n2 |
|---|---|---|---|---|
| 218-69 | A218 | B69 | 2 | 1 |
| 218-70 | A218 | B70 | 2 | 1 |
| 218-71 | A218 | B71 | 2 | 1 |
| 218-72 | A218 | B72 | 2 | 1 |
| 218-73 | A218 | B73 | 2 | 1 |
| 218-74 | A218 | B74 | 2 | 1 |
| 218-75 | A218 | B75 | 2 | 1 |
| 218-76 | A218 | B76 | 2 | 1 |
| 218-77 | A218 | B77 | 2 | 1 |
| 218-78 | A218 | B78 | 2 | 1 |
| 218-79 | A218 | B79 | 2 | 1 |
| 218-80 | A218 | B80 | 2 | 1 |
| 218-81 | A218 | B81 | 2 | 1 |
| 218-82 | A218 | B82 | 2 | 1 |
| 218-83 | A218 | B83 | 2 | 1 |
| 218-84 | A218 | B84 | 2 | 1 |
| 218-85 | A218 | B85 | 2 | 1 |
| 218-86 | A218 | B86 | 2 | 1 |
| 218-87 | A218 | B87 | 2 | 1 |
| 218-88 | A218 | B88 | 2 | 1 |
| 218-89 | A218 | B89 | 2 | 1 |
| 218-90 | A218 | B90 | 2 | 1 |
| 218-91 | A218 | B91 | 2 | 1 |
| 218-92 | A218 | B92 | 2 | 1 |
| 218-93 | A218 | B93 | 2 | 1 |
| 218-94 | A218 | B94 | 2 | 1 |
| 218-95 | A218 | B95 | 2 | 1 |
| 218-96 | A218 | B96 | 2 | 1 |
| 218-97 | A218 | B97 | 2 | 1 |
| 218-98 | A218 | B98 | 2 | 1 |

TABLE 35

| Compound No. | Ln₁ | Ln₂ | n1 | n2 |
|---|---|---|---|---|
| 218-99 | A218 | B99 | 2 | 1 |
| 218-100 | A218 | B100 | 2 | 1 |
| 218-101 | A218 | B101 | 2 | 1 |
| 218-102 | A218 | B102 | 2 | 1 |
| 218-103 | A218 | B103 | 2 | 1 |
| 218-104 | A218 | B104 | 2 | 1 |
| 218-105 | A218 | B105 | 2 | 1 |
| 218-106 | A218 | B106 | 2 | 1 |
| 218-107 | A218 | B107 | 2 | 1 |
| 218-108 | A218 | B108 | 2 | 1 |
| 218-109 | A218 | B109 | 2 | 1 |
| 218-110 | A218 | B110 | 2 | 1 |
| 218-111 | A218 | B111 | 2 | 1 |
| 218-112 | A218 | B112 | 2 | 1 |
| 218-113 | A218 | B113 | 2 | 1 |
| 218-114 | A218 | B114 | 2 | 1 |
| 218-115 | A218 | B115 | 2 | 1 |
| 218-116 | A218 | B116 | 2 | 1 |
| 218-117 | A218 | B117 | 2 | 1 |
| 218-118 | A218 | B118 | 2 | 1 |
| 218-119 | A218 | B119 | 2 | 1 |
| 218-120 | A218 | B120 | 2 | 1 |
| 218-121 | A218 | B121 | 2 | 1 |
| 218-122 | A218 | B122 | 2 | 1 |
| 218-123 | A218 | B123 | 2 | 1 |
| 218-124 | A218 | B124 | 2 | 1 |
| 218-125 | A218 | B125 | 2 | 1 |
| 218-126 | A218 | B126 | 2 | 1 |
| 218-127 | A218 | B127 | 2 | 1 |
| 218-128 | A218 | B128 | 2 | 1 |

TABLE 36

| Compound No. | Ln₁ | Ln₂ | n1 | n2 |
|---|---|---|---|---|
| 218-129 | A218 | B129 | 2 | 1 |
| 218-130 | A218 | B130 | 2 | 1 |
| 218-131 | A218 | B131 | 2 | 1 |
| 218-132 | A218 | B132 | 2 | 1 |
| 218-133 | A218 | B133 | 2 | 1 |
| 218-134 | A218 | B134 | 2 | 1 |
| 218-135 | A218 | B135 | 2 | 1 |
| 218-136 | A218 | B136 | 2 | 1 |
| 218-137 | A218 | B137 | 2 | 1 |
| 218-138 | A218 | B138 | 2 | 1 |
| 218-139 | A218 | B139 | 2 | 1 |
| 218-140 | A218 | B140 | 2 | 1 |
| 218-141 | A218 | B141 | 2 | 1 |
| 218-142 | A218 | B142 | 2 | 1 |
| 218-143 | A218 | B143 | 2 | 1 |
| 218-144 | A218 | B144 | 2 | 1 |
| 218-145 | A218 | B145 | 2 | 1 |
| 218-146 | A218 | B146 | 2 | 1 |
| 218-147 | A218 | B147 | 2 | 1 |
| 218-148 | A218 | B148 | 2 | 1 |
| 218-149 | A218 | B149 | 2 | 1 |
| 218-150 | A218 | B150 | 2 | 1 |
| 218-151 | A218 | B151 | 2 | 1 |
| 218-152 | A218 | B152 | 2 | 1 |
| 218-153 | A218 | B153 | 2 | 1 |
| 218-154 | A218 | B154 | 2 | 1 |
| 218-155 | A218 | B155 | 2 | 1 |
| 218-156 | A218 | B156 | 2 | 1 |
| 218-157 | A218 | B157 | 2 | 1 |
| 218-158 | A218 | B158 | 2 | 1 |

TABLE 37

| Compound No. | Ln₁ | Ln₂ | n1 | n2 |
|---|---|---|---|---|
| 218-159 | A218 | B159 | 2 | 1 |
| 218-160 | A218 | B160 | 2 | 1 |
| 218-161 | A218 | B161 | 2 | 1 |
| 218-162 | A218 | B162 | 2 | 1 |
| 218-163 | A218 | B163 | 2 | 1 |
| 218-164 | A218 | B164 | 2 | 1 |
| 218-165 | A218 | B165 | 2 | 1 |
| 218-166 | A218 | B166 | 2 | 1 |
| 218-167 | A218 | B167 | 2 | 1 |
| 218-168 | A218 | B168 | 2 | 1 |
| 218-169 | A218 | B169 | 2 | 1 |
| 218-170 | A218 | B170 | 2 | 1 |
| 218-171 | A218 | B171 | 2 | 1 |
| 218-172 | A218 | B172 | 2 | 1 |
| 218-173 | A218 | B173 | 2 | 1 |
| 218-174 | A218 | B174 | 2 | 1 |
| 218-175 | A218 | B175 | 2 | 1 |
| 218-176 | A218 | B176 | 2 | 1 |
| 218-177 | A218 | B177 | 2 | 1 |
| 218-178 | A218 | B178 | 2 | 1 |
| 218-179 | A218 | B179 | 2 | 1 |
| 218-180 | A218 | B180 | 2 | 1 |
| 218-181 | A218 | B181 | 2 | 1 |
| 218-182 | A218 | B182 | 2 | 1 |
| 218-183 | A218 | B183 | 2 | 1 |
| 218-184 | A218 | B184 | 2 | 1 |
| 218-185 | A218 | B185 | 2 | 1 |
| 218-186 | A218 | B186 | 2 | 1 |
| 218-187 | A218 | B187 | 2 | 1 |
| 218-188 | A218 | B188 | 2 | 1 |

TABLE 38

| Compound No. | Ln₁ | Ln₂ | n1 | n2 |
|---|---|---|---|---|
| 218-189 | A218 | B189 | 2 | 1 |
| 218-190 | A218 | B190 | 2 | 1 |
| 218-191 | A218 | B191 | 2 | 1 |
| 218-192 | A218 | B192 | 2 | 1 |
| 218-193 | A218 | B193 | 2 | 1 |
| 218-194 | A218 | B194 | 2 | 1 |
| 218-195 | A218 | B195 | 2 | 1 |
| 218-196 | A218 | B196 | 2 | 1 |
| 218-197 | A218 | B197 | 2 | 1 |
| 218-198 | A218 | B198 | 2 | 1 |
| 218-199 | A218 | B199 | 2 | 1 |
| 218-200 | A218 | B200 | 2 | 1 |
| 218-201 | A218 | B201 | 2 | 1 |
| 218-202 | A218 | B202 | 2 | 1 |
| 218-203 | A218 | B203 | 2 | 1 |
| 218-204 | A218 | B204 | 2 | 1 |
| 218-205 | A218 | B205 | 2 | 1 |
| 218-206 | A218 | B206 | 2 | 1 |
| 218-207 | A218 | B207 | 2 | 1 |
| 218-208 | A218 | B208 | 2 | 1 |
| 218-209 | A218 | B209 | 2 | 1 |
| 218-210 | A218 | B210 | 2 | 1 |
| 218-211 | A218 | B211 | 2 | 1 |
| 218-212 | A218 | B212 | 2 | 1 |
| 218-213 | A218 | B213 | 2 | 1 |
| 218-214 | A218 | B214 | 2 | 1 |
| 218-215 | A218 | B215 | 2 | 1 |
| 218-216 | A218 | B216 | 2 | 1 |
| 218-217 | A218 | B217 | 2 | 1 |
| 218-218 | A218 | B218 | 2 | 1 |

TABLE 39

| Compound No. | Ln₁ | Ln₂ | n1 | n2 |
|---|---|---|---|---|
| 218-219 | A218 | B219 | 2 | 1 |
| 218-220 | A218 | B220 | 2 | 1 |
| 218-221 | A218 | B221 | 2 | 1 |
| 218-222 | A218 | B222 | 2 | 1 |
| 218-223 | A218 | B223 | 2 | 1 |
| 218-224 | A218 | B224 | 2 | 1 |
| 218-225 | A218 | B225 | 2 | 1 |
| 218-226 | A218 | B226 | 2 | 1 |
| 218-227 | A218 | B227 | 2 | 1 |
| 218-228 | A218 | B228 | 2 | 1 |
| 218-229 | A218 | B229 | 2 | 1 |
| 218-230 | A218 | B230 | 2 | 1 |
| 218-231 | A218 | B231 | 2 | 1 |
| 218-232 | A218 | B232 | 2 | 1 |
| 218-233 | A218 | B233 | 2 | 1 |
| 218-234 | A218 | B234 | 2 | 1 |
| 218-235 | A218 | B235 | 2 | 1 |
| 218-236 | A218 | B236 | 2 | 1 |
| 218-237 | A218 | B237 | 2 | 1 |
| 218-238 | A218 | B238 | 2 | 1 |
| 218-239 | A218 | B239 | 2 | 1 |
| 218-240 | A218 | B240 | 2 | 1 |
| 218-241 | A218 | B241 | 2 | 1 |
| 218-242 | A218 | B242 | 2 | 1 |
| 218-243 | A218 | B243 | 2 | 1 |
| 218-244 | A218 | B244 | 2 | 1 |
| 218-245 | A218 | B245 | 2 | 1 |
| 218-246 | A218 | B246 | 2 | 1 |
| 218-247 | A218 | B247 | 2 | 1 |
| 218-248 | A218 | B248 | 2 | 1 |

TABLE 40

| Compound No. | Ln₁ | Ln₂ | n1 | n2 |
|---|---|---|---|---|
| 218-249 | A218 | B249 | 2 | 1 |
| 218-250 | A218 | B250 | 2 | 1 |
| 218-251 | A218 | B251 | 2 | 1 |
| 218-252 | A218 | B252 | 2 | 1 |
| 218-253 | A218 | B253 | 2 | 1 |
| 218-254 | A218 | B254 | 2 | 1 |
| 218-255 | A218 | B255 | 2 | 1 |
| 218-256 | A218 | B256 | 2 | 1 |
| 218-257 | A218 | B257 | 2 | 1 |
| 218-258 | A218 | B258 | 2 | 1 |
| 218-259 | A218 | B259 | 2 | 1 |
| 218-260 | A218 | B260 | 2 | 1 |
| 218-261 | A218 | B261 | 2 | 1 |
| 218-262 | A218 | B262 | 2 | 1 |
| 218-263 | A218 | B263 | 2 | 1 |
| 218-264 | A218 | B264 | 2 | 1 |
| 218-265 | A218 | B265 | 2 | 1 |
| 218-266 | A218 | B266 | 2 | 1 |
| 218-267 | A218 | B267 | 2 | 1 |
| 218-268 | A218 | B268 | 2 | 1 |
| 218-269 | A218 | B269 | 2 | 1 |
| 218-270 | A218 | B270 | 2 | 1 |
| 218-271 | A218 | B271 | 2 | 1 |
| 218-272 | A218 | B272 | 2 | 1 |
| 218-273 | A218 | B273 | 2 | 1 |
| 218-274 | A218 | B274 | 2 | 1 |
| 218-275 | A218 | B275 | 2 | 1 |
| 218-276 | A218 | B276 | 2 | 1 |
| 218-277 | A218 | B277 | 2 | 1 |
| 218-278 | A218 | B278 | 2 | 1 |

TABLE 41

| Compound No. | Ln₁ | Ln₂ | n1 | n2 |
|---|---|---|---|---|
| 218-279 | A218 | B279 | 2 | 1 |
| 218-280 | A218 | B280 | 2 | 1 |
| 241-303 | A241 | B303 | 2 | 1 |
| 242-304 | A242 | B304 | 2 | 1 |
| 243-305 | A243 | B305 | 2 | 1 |
| 244-306 | A244 | B306 | 2 | 1 |
| 244-307 | A244 | B307 | 2 | 1 |
| 244-308 | A244 | B308 | 2 | 1 |

TABLE 42

| Compound No. | Ln₁ | Ln₂ | n1 | n2 |
|---|---|---|---|---|
| 245-2 | A245 | B2 | 2 | 1 |
| 245-3 | A245 | B3 | 2 | 1 |
| 245-4 | A245 | B4 | 2 | 1 |
| 245-5 | A245 | B5 | 2 | 1 |
| 245-6 | A245 | B6 | 2 | 1 |
| 245-7 | A245 | B7 | 2 | 1 |
| 245-8 | A245 | B8 | 2 | 1 |
| 245-9 | A245 | B9 | 2 | 1 |
| 245-10 | A245 | B10 | 2 | 1 |
| 245-11 | A245 | B11 | 2 | 1 |
| 245-12 | A245 | B12 | 2 | 1 |
| 245-13 | A245 | B13 | 2 | 1 |
| 245-14 | A245 | B14 | 2 | 1 |
| 245-15 | A245 | B15 | 2 | 1 |
| 245-16 | A245 | B16 | 2 | 1 |
| 245-17 | A245 | B17 | 2 | 1 |
| 245-18 | A245 | B18 | 2 | 1 |
| 245-19 | A245 | B19 | 2 | 1 |
| 245-20 | A245 | B20 | 2 | 1 |
| 245-21 | A245 | B21 | 2 | 1 |
| 245-22 | A245 | B22 | 2 | 1 |
| 245-23 | A245 | B23 | 2 | 1 |
| 245-24 | A245 | B24 | 2 | 1 |
| 245-25 | A245 | B25 | 2 | 1 |
| 245-26 | A245 | B26 | 2 | 1 |
| 245-27 | A245 | B27 | 2 | 1 |
| 245-28 | A245 | B28 | 2 | 1 |
| 245-29 | A245 | B29 | 2 | 1 |
| 245-30 | A245 | B30 | 2 | 1 |
| 245-31 | A245 | B31 | 2 | 1 |

TABLE 43

| Compound No. | Ln₁ | Ln₂ | n1 | n2 |
|---|---|---|---|---|
| 245-32 | A245 | B32 | 2 | 1 |
| 245-33 | A245 | B33 | 2 | 1 |
| 245-34 | A245 | B34 | 2 | 1 |
| 245-35 | A245 | B35 | 2 | 1 |
| 245-36 | A245 | B36 | 2 | 1 |
| 245-37 | A245 | B37 | 2 | 1 |
| 245-38 | A245 | B38 | 2 | 1 |
| 245-39 | A245 | B39 | 2 | 1 |
| 245-40 | A245 | B40 | 2 | 1 |
| 245-41 | A245 | B41 | 2 | 1 |
| 245-42 | A245 | B42 | 2 | 1 |
| 245-43 | A245 | B43 | 2 | 1 |
| 245-44 | A245 | B44 | 2 | 1 |
| 245-45 | A245 | B45 | 2 | 1 |
| 245-46 | A245 | B46 | 2 | 1 |
| 245-47 | A245 | B47 | 2 | 1 |
| 245-48 | A245 | B48 | 2 | 1 |
| 245-49 | A245 | B49 | 2 | 1 |
| 245-50 | A245 | B50 | 2 | 1 |
| 245-51 | A245 | B51 | 2 | 1 |
| 245-52 | A245 | B52 | 2 | 1 |
| 245-53 | A245 | B53 | 2 | 1 |
| 245-54 | A245 | B54 | 2 | 1 |
| 245-56 | A245 | B56 | 2 | 1 |
| 245-57 | A245 | B57 | 2 | 1 |
| 245-58 | A245 | B58 | 2 | 1 |
| 245-59 | A245 | B59 | 2 | 1 |
| 245-60 | A245 | B60 | 2 | 1 |
| 245-61 | A245 | B61 | 2 | 1 |
| 245-62 | A245 | B62 | 2 | 1 |

TABLE 44

| Compound No. | Ln₁ | Ln₂ | n1 | n2 |
|---|---|---|---|---|
| 245-63 | A245 | B63 | 2 | 1 |
| 245-64 | A245 | B64 | 2 | 1 |
| 245-65 | A245 | B65 | 2 | 1 |
| 245-66 | A245 | B66 | 2 | 1 |
| 245-67 | A245 | B67 | 2 | 1 |
| 245-68 | A245 | B68 | 2 | 1 |
| 245-69 | A245 | B69 | 2 | 1 |
| 245-70 | A245 | B70 | 2 | 1 |
| 245-71 | A245 | B71 | 2 | 1 |
| 245-72 | A245 | B72 | 2 | 1 |
| 245-73 | A245 | B73 | 2 | 1 |
| 245-74 | A245 | B74 | 2 | 1 |
| 245-75 | A245 | B75 | 2 | 1 |
| 245-76 | A245 | B76 | 2 | 1 |
| 245-77 | A245 | B77 | 2 | 1 |
| 245-78 | A245 | B78 | 2 | 1 |
| 245-79 | A245 | B79 | 2 | 1 |
| 245-80 | A245 | B80 | 2 | 1 |
| 245-81 | A245 | B81 | 2 | 1 |
| 245-82 | A245 | B82 | 2 | 1 |
| 245-83 | A245 | B83 | 2 | 1 |
| 245-84 | A245 | B84 | 2 | 1 |
| 245-85 | A245 | B85 | 2 | 1 |
| 245-86 | A245 | B86 | 2 | 1 |
| 245-87 | A245 | B87 | 2 | 1 |
| 245-88 | A245 | B88 | 2 | 1 |
| 245-89 | A245 | B89 | 2 | 1 |
| 245-90 | A245 | B90 | 2 | 1 |
| 245-91 | A245 | B91 | 2 | 1 |
| 245-92 | A245 | B92 | 2 | 1 |

TABLE 45

| Compound No. | Ln₁ | Ln₂ | n1 | n2 |
|---|---|---|---|---|
| 245-93 | A245 | B93 | 2 | 1 |
| 245-94 | A245 | B94 | 2 | 1 |
| 245-95 | A245 | B95 | 2 | 1 |
| 245-96 | A245 | B96 | 2 | 1 |
| 245-97 | A245 | B97 | 2 | 1 |
| 245-98 | A245 | B98 | 2 | 1 |
| 245-99 | A245 | B99 | 2 | 1 |
| 245-100 | A245 | B100 | 2 | 1 |
| 245-101 | A245 | B101 | 2 | 1 |
| 245-102 | A245 | B102 | 2 | 1 |
| 245-103 | A245 | B103 | 2 | 1 |
| 245-104 | A245 | B104 | 2 | 1 |
| 245-105 | A245 | B105 | 2 | 1 |
| 245-106 | A245 | B106 | 2 | 1 |
| 245-107 | A245 | B107 | 2 | 1 |
| 245-108 | A245 | B108 | 2 | 1 |
| 245-109 | A245 | B109 | 2 | 1 |
| 245-110 | A245 | B110 | 2 | 1 |
| 245-111 | A245 | B111 | 2 | 1 |
| 245-112 | A245 | B112 | 2 | 1 |
| 245-113 | A245 | B113 | 2 | 1 |
| 245-114 | A245 | B114 | 2 | 1 |
| 245-115 | A245 | B115 | 2 | 1 |
| 245-116 | A245 | B116 | 2 | 1 |
| 245-117 | A245 | B117 | 2 | 1 |
| 245-118 | A245 | B118 | 2 | 1 |
| 245-119 | A245 | B119 | 2 | 1 |
| 245-120 | A245 | B120 | 2 | 1 |
| 245-121 | A245 | B121 | 2 | 1 |
| 245-122 | A245 | B122 | 2 | 1 |

TABLE 46

| Compound No. | Ln₁ | Ln₂ | n1 | n2 |
|---|---|---|---|---|
| 245-123 | A245 | B123 | 2 | 1 |
| 245-124 | A245 | B124 | 2 | 1 |
| 245-125 | A245 | B125 | 2 | 1 |
| 245-126 | A245 | B126 | 2 | 1 |
| 245-127 | A245 | B127 | 2 | 1 |
| 245-128 | A245 | B128 | 2 | 1 |
| 245-129 | A245 | B129 | 2 | 1 |
| 245-130 | A245 | B130 | 2 | 1 |
| 245-131 | A245 | B131 | 2 | 1 |
| 245-132 | A245 | B132 | 2 | 1 |
| 245-133 | A245 | B133 | 2 | 1 |
| 245-134 | A245 | B134 | 2 | 1 |
| 245-135 | A245 | B135 | 2 | 1 |
| 245-136 | A245 | B136 | 2 | 1 |
| 245-137 | A245 | B137 | 2 | 1 |
| 245-138 | A245 | B138 | 2 | 1 |
| 245-139 | A245 | B139 | 2 | 1 |
| 245-140 | A245 | B140 | 2 | 1 |
| 245-141 | A245 | B141 | 2 | 1 |
| 245-142 | A245 | B142 | 2 | 1 |
| 245-143 | A245 | B143 | 2 | 1 |
| 245-144 | A245 | B144 | 2 | 1 |
| 245-145 | A245 | B145 | 2 | 1 |
| 245-146 | A245 | B146 | 2 | 1 |
| 245-147 | A245 | B147 | 2 | 1 |
| 245-148 | A245 | B148 | 2 | 1 |
| 245-149 | A245 | B149 | 2 | 1 |
| 245-150 | A245 | B150 | 2 | 1 |
| 245-151 | A245 | B151 | 2 | 1 |
| 245-152 | A245 | B152 | 2 | 1 |

TABLE 47

| Compound No. | Ln₁ | Ln₂ | n1 | n2 |
|---|---|---|---|---|
| 245-153 | A245 | B153 | 2 | 1 |
| 245-154 | A245 | B154 | 2 | 1 |
| 245-155 | A245 | B155 | 2 | 1 |
| 245-156 | A245 | B156 | 2 | 1 |
| 245-157 | A245 | B157 | 2 | 1 |
| 245-158 | A245 | B158 | 2 | 1 |
| 245-159 | A245 | B159 | 2 | 1 |
| 245-160 | A245 | B160 | 2 | 1 |
| 245-161 | A245 | B161 | 2 | 1 |
| 245-162 | A245 | B162 | 2 | 1 |
| 245-163 | A245 | B163 | 2 | 1 |
| 245-164 | A245 | B164 | 2 | 1 |
| 245-165 | A245 | B165 | 2 | 1 |
| 245-166 | A245 | B166 | 2 | 1 |
| 245-167 | A245 | B167 | 2 | 1 |
| 245-168 | A245 | B168 | 2 | 1 |
| 245-169 | A245 | B169 | 2 | 1 |
| 245-170 | A245 | B170 | 2 | 1 |
| 245-171 | A245 | B171 | 2 | 1 |
| 245-172 | A245 | B172 | 2 | 1 |
| 245-173 | A245 | B173 | 2 | 1 |
| 245-174 | A245 | B174 | 2 | 1 |
| 245-175 | A245 | B175 | 2 | 1 |
| 245-176 | A245 | B176 | 2 | 1 |
| 245-177 | A245 | B177 | 2 | 1 |
| 245-178 | A245 | B178 | 2 | 1 |
| 245-179 | A245 | B179 | 2 | 1 |
| 245-180 | A245 | B180 | 2 | 1 |
| 245-181 | A245 | B181 | 2 | 1 |
| 245-182 | A245 | B182 | 2 | 1 |

TABLE 48

| Compound No. | Ln1 | Ln2 | n1 | n2 |
|---|---|---|---|---|
| 245-183 | A245 | B183 | 2 | 1 |
| 245-184 | A245 | B184 | 2 | 1 |
| 245-185 | A245 | B185 | 2 | 1 |
| 245-186 | A245 | B186 | 2 | 1 |
| 245-187 | A245 | B187 | 2 | 1 |
| 245-188 | A245 | B188 | 2 | 1 |
| 245-189 | A245 | B189 | 2 | 1 |
| 245-190 | A245 | B190 | 2 | 1 |
| 245-191 | A245 | B191 | 2 | 1 |
| 245-192 | A245 | B192 | 2 | 1 |
| 245-193 | A245 | B193 | 2 | 1 |
| 245-194 | A245 | B194 | 2 | 1 |
| 245-195 | A245 | B195 | 2 | 1 |
| 245-196 | A245 | B196 | 2 | 1 |
| 245-197 | A245 | B197 | 2 | 1 |
| 245-198 | A245 | B198 | 2 | 1 |
| 245-199 | A245 | B199 | 2 | 1 |
| 245-200 | A245 | B200 | 2 | 1 |
| 245-201 | A245 | B201 | 2 | 1 |
| 245-202 | A245 | B202 | 2 | 1 |
| 245-203 | A245 | B203 | 2 | 1 |
| 245-204 | A245 | B204 | 2 | 1 |
| 245-205 | A245 | B205 | 2 | 1 |
| 245-206 | A245 | B206 | 2 | 1 |
| 245-207 | A245 | B207 | 2 | 1 |
| 245-208 | A245 | B208 | 2 | 1 |
| 245-209 | A245 | B209 | 2 | 1 |
| 245-210 | A245 | B210 | 2 | 1 |
| 245-211 | A245 | B211 | 2 | 1 |
| 245-212 | A245 | B212 | 2 | 1 |

TABLE 49

| Compound No. | Ln1 | Ln2 | n1 | n2 |
|---|---|---|---|---|
| 245-213 | A245 | B213 | 2 | 1 |
| 245-214 | A245 | B214 | 2 | 1 |
| 245-215 | A245 | B215 | 2 | 1 |
| 245-216 | A245 | B216 | 2 | 1 |
| 245-217 | A245 | B217 | 2 | 1 |
| 245-218 | A245 | B218 | 2 | 1 |
| 245-219 | A245 | B219 | 2 | 1 |
| 245-220 | A245 | B220 | 2 | 1 |
| 245-221 | A245 | B221 | 2 | 1 |
| 245-222 | A245 | B222 | 2 | 1 |
| 245-223 | A245 | B223 | 2 | 1 |
| 245-224 | A245 | B224 | 2 | 1 |
| 245-225 | A245 | B225 | 2 | 1 |
| 245-226 | A245 | B226 | 2 | 1 |
| 245-227 | A245 | B227 | 2 | 1 |
| 245-228 | A245 | B228 | 2 | 1 |
| 245-229 | A245 | B229 | 2 | 1 |
| 245-230 | A245 | B230 | 2 | 1 |
| 245-231 | A245 | B231 | 2 | 1 |
| 245-232 | A245 | B232 | 2 | 1 |
| 245-233 | A245 | B233 | 2 | 1 |
| 245-234 | A245 | B234 | 2 | 1 |
| 245-235 | A245 | B235 | 2 | 1 |
| 245-236 | A245 | B236 | 2 | 1 |
| 245-237 | A245 | B237 | 2 | 1 |
| 245-238 | A245 | B238 | 2 | 1 |
| 245-239 | A245 | B239 | 2 | 1 |
| 245-240 | A245 | B240 | 2 | 1 |
| 245-241 | A245 | B241 | 2 | 1 |
| 245-242 | A245 | B242 | 2 | 1 |

TABLE 50

| Compound No. | Ln1 | Ln2 | n1 | n2 |
|---|---|---|---|---|
| 245-243 | A245 | B243 | 2 | 1 |
| 245-244 | A245 | B244 | 2 | 1 |
| 245-245 | A245 | B245 | 2 | 1 |
| 245-246 | A245 | B246 | 2 | 1 |
| 245-247 | A245 | B247 | 2 | 1 |
| 245-248 | A245 | B248 | 2 | 1 |
| 245-249 | A245 | B249 | 2 | 1 |
| 245-250 | A245 | B250 | 2 | 1 |
| 245-251 | A245 | B251 | 2 | 1 |
| 245-252 | A245 | B252 | 2 | 1 |
| 245-253 | A245 | B253 | 2 | 1 |
| 245-254 | A245 | B254 | 2 | 1 |
| 245-255 | A245 | B255 | 2 | 1 |
| 245-256 | A245 | B256 | 2 | 1 |
| 245-257 | A245 | B257 | 2 | 1 |
| 245-258 | A245 | B258 | 2 | 1 |
| 245-259 | A245 | B259 | 2 | 1 |
| 245-260 | A245 | B260 | 2 | 1 |
| 245-261 | A245 | B261 | 2 | 1 |
| 245-262 | A245 | B262 | 2 | 1 |
| 245-263 | A245 | B263 | 2 | 1 |
| 245-264 | A245 | B264 | 2 | 1 |
| 245-265 | A245 | B265 | 2 | 1 |
| 245-266 | A245 | B266 | 2 | 1 |
| 245-267 | A245 | B267 | 2 | 1 |
| 245-268 | A245 | B268 | 2 | 1 |
| 245-269 | A245 | B269 | 2 | 1 |
| 245-270 | A245 | B270 | 2 | 1 |
| 245-271 | A245 | B271 | 2 | 1 |
| 245-272 | A245 | B272 | 2 | 1 |
| 245-243 | A245 | B243 | 2 | 1 |

TABLE 51

| Compound No. | Ln1 | Ln2 | n1 | n2 |
|---|---|---|---|---|
| 245-273 | A245 | B273 | 2 | 1 |
| 245-274 | A245 | B274 | 2 | 1 |
| 245-275 | A245 | B275 | 2 | 1 |
| 245-276 | A245 | B276 | 2 | 1 |
| 245-277 | A245 | B277 | 2 | 1 |
| 245-278 | A245 | B278 | 2 | 1 |
| 245-279 | A245 | B279 | 2 | 1 |
| 245-280 | A245 | B280 | 2 | 1 |
| 245-281 | A245 | B281 | 2 | 1 |
| 245-282 | A245 | B282 | 2 | 1 |
| 245-283 | A245 | B283 | 2 | 1 |
| 245-284 | A245 | B284 | 2 | 1 |
| 245-285 | A245 | B285 | 2 | 1 |
| 245-286 | A245 | B286 | 2 | 1 |
| 245-287 | A245 | B287 | 2 | 1 |
| 245-288 | A245 | B288 | 2 | 1 |
| 245-289 | A245 | B289 | 2 | 1 |
| 245-290 | A245 | B290 | 2 | 1 |
| 245-291 | A245 | B291 | 2 | 1 |
| 245-292 | A245 | B292 | 2 | 1 |
| 245-293 | A245 | B293 | 2 | 1 |
| 245-294 | A245 | B294 | 2 | 1 |
| 245-295 | A245 | B295 | 2 | 1 |
| 245-296 | A245 | B296 | 2 | 1 |
| 245-297 | A245 | B297 | 2 | 1 |
| 245-298 | A245 | B298 | 2 | 1 |
| 245-299 | A245 | B299 | 2 | 1 |
| 245-300 | A245 | B300 | 2 | 1 |
| 245-301 | A245 | B301 | 2 | 1 |
| 245-302 | A245 | B302 | 2 | 1 |

TABLE 52

| Compound No. | Ln1 | Ln2 | n1 | n2 |
|---|---|---|---|---|
| 245-303 | A245 | B303 | 2 | 1 |
| 245-304 | A245 | B304 | 2 | 1 |
| 245-305 | A245 | B305 | 2 | 1 |
| 245-306 | A245 | B306 | 2 | 1 |
| 245-307 | A245 | B307 | 2 | 1 |
| 245-308 | A245 | B308 | 2 | 1 |
| 245-309 | A245 | B309 | 2 | 1 |
| 245-310 | A245 | B310 | 2 | 1 |
| 245-311 | A245 | B311 | 2 | 1 |
| 245-312 | A245 | B312 | 2 | 1 |
| 245-313 | A245 | B313 | 2 | 1 |
| 245-314 | A245 | B314 | 2 | 1 |
| 245-315 | A245 | B315 | 2 | 1 |
| 245-316 | A245 | B316 | 2 | 1 |
| 245-317 | A245 | B317 | 2 | 1 |
| 245-318 | A245 | B318 | 2 | 1 |
| 245-319 | A245 | B319 | 2 | 1 |
| 245-320 | A245 | B320 | 2 | 1 |
| 245-321 | A245 | B321 | 2 | 1 |
| 245-322 | A245 | B322 | 2 | 1 |
| 245-323 | A245 | B323 | 2 | 1 |
| 245-324 | A245 | B324 | 2 | 1 |
| 245-325 | A245 | B325 | 2 | 1 |
| 245-326 | A245 | B326 | 2 | 1 |
| 245-327 | A245 | B327 | 2 | 1 |
| 245-328 | A245 | B328 | 2 | 1 |
| 245-329 | A245 | B329 | 2 | 1 |
| 245-330 | A245 | B330 | 2 | 1 |
| 245-331 | A245 | B331 | 2 | 1 |
| 245-332 | A245 | B332 | 2 | 1 |

TABLE 53

| Compound No. | Ln1 | Ln2 | n1 | n2 |
|---|---|---|---|---|
| 245-333 | A245 | B333 | 2 | 1 |
| 245-334 | A245 | B334 | 2 | 1 |
| 245-335 | A245 | B335 | 2 | 1 |
| 245-336 | A245 | B336 | 2 | 1 |
| 245-337 | A245 | B337 | 2 | 1 |
| 245-338 | A245 | B338 | 2 | 1 |
| 245-339 | A245 | B339 | 2 | 1 |
| 245-340 | A245 | B340 | 2 | 1 |
| 245-341 | A245 | B341 | 2 | 1 |
| 245-342 | A245 | B342 | 2 | 1 |
| 245-343 | A245 | B343 | 2 | 1 |
| 245-344 | A245 | B344 | 2 | 1 |
| 245-345 | A245 | B345 | 2 | 1 |
| 245-346 | A245 | B346 | 2 | 1 |
| 245-347 | A245 | B347 | 2 | 1 |
| 245-348 | A245 | B348 | 2 | 1 |
| 245-349 | A245 | B349 | 2 | 1 |
| 245-350 | A245 | B350 | 2 | 1 |
| 245-351 | A245 | B351 | 2 | 1 |
| 245-352 | A245 | B352 | 2 | 1 |
| 245-353 | A245 | B353 | 2 | 1 |
| 245-354 | A245 | B354 | 2 | 1 |
| 245-355 | A245 | B355 | 2 | 1 |
| 245-356 | A245 | B356 | 2 | 1 |
| 245-357 | A245 | B357 | 2 | 1 |
| 245-358 | A245 | B358 | 2 | 1 |
| 245-359 | A245 | B359 | 2 | 1 |
| 245-360 | A245 | B360 | 2 | 1 |
| 245-361 | A245 | B361 | 2 | 1 |
| 245-362 | A245 | B362 | 2 | 1 |

TABLE 54

| Compound No. | Ln1 | Ln2 | n1 | n2 |
|---|---|---|---|---|
| 245-363 | A245 | B363 | 2 | 1 |
| 245-364 | A245 | B364 | 2 | 1 |
| 245-365 | A245 | B365 | 2 | 1 |
| 245-366 | A245 | B366 | 2 | 1 |
| 245-367 | A245 | B367 | 2 | 1 |
| 245-368 | A245 | B368 | 2 | 1 |
| 245-369 | A245 | B369 | 2 | 1 |
| 245-370 | A245 | B370 | 2 | 1 |
| 245-371 | A245 | B371 | 2 | 1 |
| 245-372 | A245 | B372 | 2 | 1 |
| 245-373 | A245 | B373 | 2 | 1 |
| 245-374 | A245 | B374 | 2 | 1 |
| 245-375 | A245 | B375 | 2 | 1 |
| 245-376 | A245 | B376 | 2 | 1 |
| 245-377 | A245 | B377 | 2 | 1 |
| 245-378 | A245 | B378 | 2 | 1 |
| 245-379 | A245 | B379 | 2 | 1 |
| 245-380 | A245 | B380 | 2 | 1 |
| 245-381 | A245 | B381 | 2 | 1 |
| 245-382 | A245 | B382 | 2 | 1 |
| 245-383 | A245 | B383 | 2 | 1 |
| 245-384 | A245 | B384 | 2 | 1 |
| 245-385 | A245 | B385 | 2 | 1 |
| 245-386 | A245 | B386 | 2 | 1 |
| 245-387 | A245 | B387 | 2 | 1 |
| 245-388 | A245 | B388 | 2 | 1 |
| 245-389 | A245 | B389 | 2 | 1 |
| 245-390 | A245 | B390 | 2 | 1 |
| 245-391 | A245 | B391 | 2 | 1 |
| 245-392 | A245 | B392 | 2 | 1 |

TABLE 55

| Compound No. | Ln1 | Ln2 | n1 | n2 |
|---|---|---|---|---|
| 245-393 | A245 | B393 | 2 | 1 |
| 245-394 | A245 | B394 | 2 | 1 |
| 245-395 | A245 | B395 | 2 | 1 |
| 245-396 | A245 | B396 | 2 | 1 |
| 245-397 | A245 | B397 | 2 | 1 |
| 245-398 | A245 | B398 | 2 | 1 |
| 245-399 | A245 | B399 | 2 | 1 |
| 245-400 | A245 | B400 | 2 | 1 |
| 245-401 | A245 | B401 | 2 | 1 |
| 245-402 | A245 | B402 | 2 | 1 |
| 245-403 | A245 | B403 | 2 | 1 |
| 245-404 | A245 | B404 | 2 | 1 |
| 245-405 | A245 | B405 | 2 | 1 |
| 245-406 | A245 | B406 | 2 | 1 |
| 245-407 | A245 | B407 | 2 | 1 |
| 245-408 | A245 | B408 | 2 | 1 |
| 245-409 | A245 | B409 | 2 | 1 |
| 245-410 | A245 | B410 | 2 | 1 |
| 245-411 | A245 | B411 | 2 | 1 |
| 245-412 | A245 | B412 | 2 | 1 |
| 245-413 | A245 | B413 | 2 | 1 |
| 245-414 | A245 | B414 | 2 | 1 |
| 245-415 | A245 | B415 | 2 | 1 |
| 245-416 | A245 | B416 | 2 | 1 |
| 245-417 | A245 | B417 | 2 | 1 |
| 245-418 | A245 | B418 | 2 | 1 |
| 245-419 | A245 | B419 | 2 | 1 |
| 245-420 | A245 | B420 | 2 | 1 |
| 265-63 | A265 | B63 | 2 | 1 |
| 265-142 | A265 | B142 | 2 | 1 |

TABLE 56

| Compound No. | Ln₁ | Ln₂ | n1 | n2 |
|---|---|---|---|---|
| 2-2N | A2 | B2 | 1 | 2 |
| 4-2N | A4 | B2 | 1 | 2 |
| 5-7N | A5 | B7 | 1 | 2 |
| 11-9N | A11 | B9 | 1 | 2 |
| 13-11N | A15 | B11 | 1 | 2 |
| 27-20N | A27 | B20 | 1 | 2 |
| 28-22N | A28 | B22 | 1 | 2 |
| 66-24N | A66 | B24 | 1 | 2 |
| 153-29N | A153 | B29 | 1 | 2 |
| 163-35N | A163 | B35 | 1 | 2 |
| 176-47N | A176 | B47 | 1 | 2 |
| 188-49N | A188 | B49 | 1 | 2 |
| 190-137N | A190 | B137 | 1 | 2 |
| 201-162N | A201 | B162 | 1 | 2 |
| 213-187N | A203 | B187 | 1 | 2 |
| 226-200N | A226 | B200 | 1 | 2 |
| 235-201N | A235 | B201 | 1 | 2 |
| 240-202N | A240 | B202 | 1 | 2 |
| 208-1N | A208 | B1 | 1 | 2 |
| 214-22N | A214 | B22 | 1 | 2 |
| 223-7N | A223 | B7 | 1 | 2 |
| 251-9N | A251 | B9 | 1 | 2 |
| 276-284N | A276 | B284 | 1 | 2 |
| 280-285N | A280 | B285 | 1 | 2 |
| 271-283N | A271 | B283 | 1 | 2 |

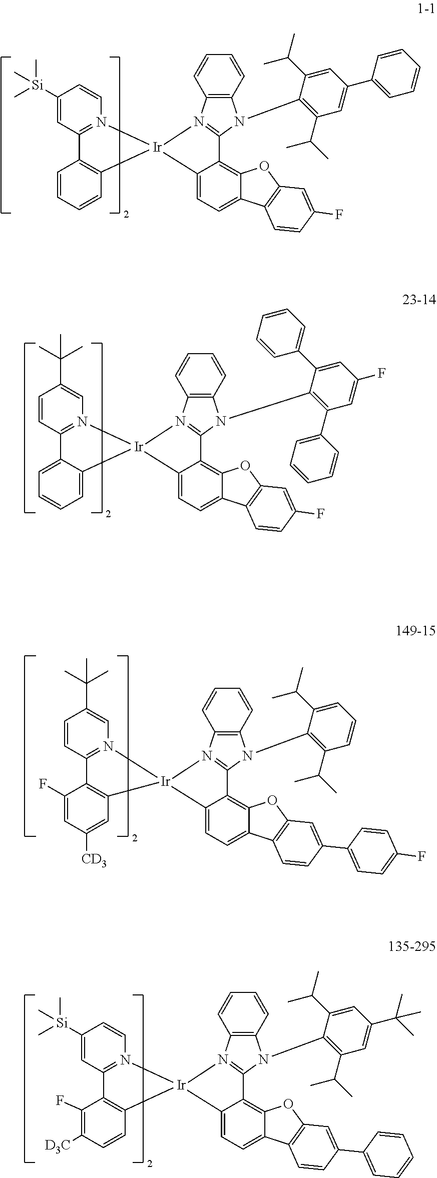

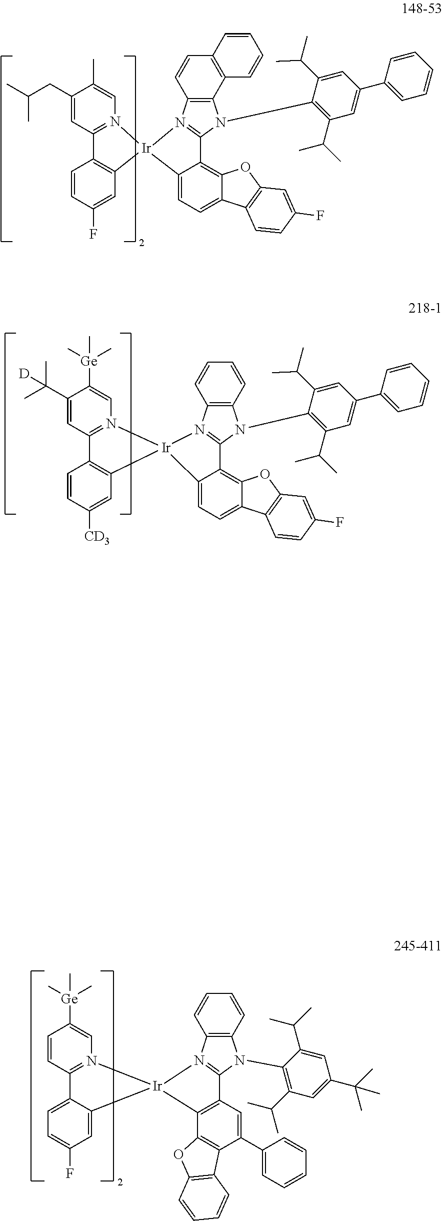

In one or more embodiments, the organometallic compound may be electrically neutral.

The organometallic compound represented by Formula 1 satisfies the structure of Formula 1 previously described, in which at least one —F is substituted into a ligand. Through this structure, the organometallic compound represented Formula 1 may reduce hole trapping through controlling the HOMO (Highest Occupied Molecular Orbital) energy level, and thereby improving the electric mobility. Therefore, the electronic device including the organometallic compound, for example, the organic light-emitting device, may show low driving voltage and high efficiency, and roll-off phenomenon may be decreased.

The highest occupied molecular orbital (HOMO) energy level, lowest unoccupied molecular orbital (LUMO) energy level, and lowest triplet excited state (Ti) energy level of selected organometallic compounds represented by Formula 1 were evaluated by density functional theory (DFT) using the Gaussian 09 program with the molecular structure optimization obtained at the B3LYP level. The results thereof are shown in Table 57, where the energy levels are in electron volts (eV).

TABLE 57

| Compound No. | HOMO (eV) | LUMO (eV) | T₁ (eV) |
|---|---|---|---|
| Compound 1-1 | −4.763 | −1.258 | 2.522 |
| Compound 23-14 | −4.760 | −1.188 | 2.512 |
| Compound 149-15 | −4.855 | −1.259 | 2.496 |
| Compound 135-295 | −4.800 | −1.293 | 2.497 |
| Compound 148-53 | −4.817 | −1.165 | 2.550 |
| Compound 218-1 | −4.739 | −1.219 | 2.545 |
| Compound 245-411 | −4.839 | −1.262 | 2.507 |

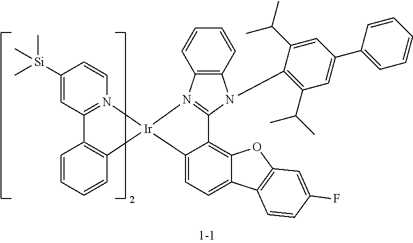

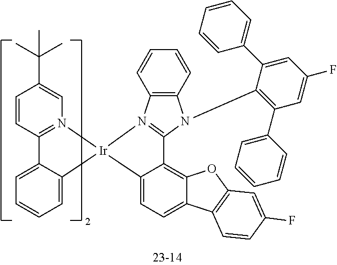

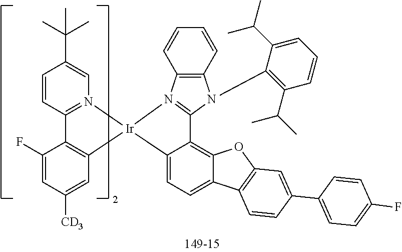

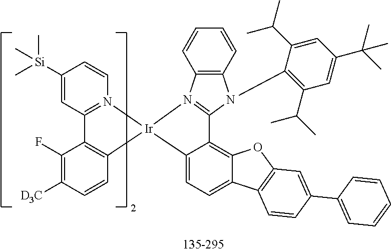

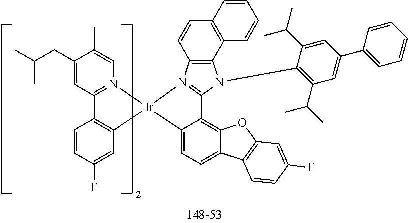

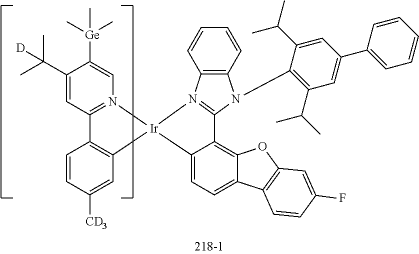

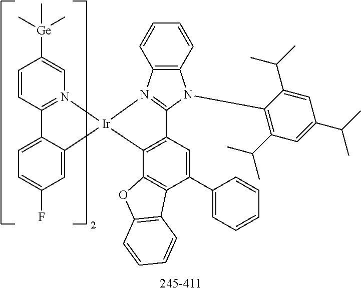

From Table 57 shows that the organometallic compound represented by Formula 1 has such electric characteristics that are suitable for use as a dopant for an electric device, for example, an organic light-emitting device.

In one or more embodiments, the full width at half maximum (FWHM) of the emission peak of the emission spectrum or the electroluminescence spectrum of the organometallic compound may be 70 nm or less. For example, the FWHM of the emission peak of the emission spectrum or the electroluminescence spectrum of the organometallic compound may be from about 30 nm to about 65 nm, from about 40 nm to about 63 nm, or from about 45 nm to about 62 nm.

In one or more embodiments, the maximum emission wavelength (emission peak wavelength, λₘₐₓ) of the emission peak of the emission spectrum or electroluminescence spectrum of the organometallic compound may be from about 500 nm to about 600 nm.

Synthesis methods of the organometallic compound represented by Formula 1 may be recognizable by one of ordinary skill in the art by referring to Synthesis Examples provided below.

The organometallic compound represented by Formula 1 is suitable for use in an organic layer of an organic light-emitting device, for example, for use as a dopant in an emission layer of the organic layer. Thus, another aspect provides an organic light-emitting device that includes: a first electrode; a second electrode; and an organic layer that is located between the first electrode and the second electrode and includes an emission layer, wherein the organic layer includes at least one organometallic compound represented by Formula 1.

As described above, due to the inclusion of the organic layer including the organometallic compound represented by Formula 1, the organic light-emitting device may have excellent characteristics in terms of driving voltage, current efficiency, power efficiency, external quantum efficiency, lifespan, and/or color purity. Also, such an organic light-emitting device may have a reduced roll-off phenomenon and a relatively narrow electroluminescent (EL) spectrum emission peak FWHM.

The organometallic compound represented by Formula 1 may be used between a pair of electrodes of an organic light-emitting device. For example, the organometallic compound represented by Formula 1 may be included in the emission layer. In this regard, the organometallic compound may act as a dopant, and the emission layer may further include a host (that is, an amount of the organometallic compound represented by Formula 1 in the emission layer is smaller than an amount of the host).

In one or more embodiments, the emission layer may emit green light. For example, the emission layer may emit green light having a maximum emission wavelength of about 500 nm to about 600 nm.

The expression “(an organic layer) includes at least one of organometallic compounds” used herein may include a case in which “(an organic layer) includes identical organometallic compounds represented by Formula 1” and a case in which “(an organic layer) includes two or more different organometallic compounds represented by Formula 1.”

For example, the organic layer may include, as the organometallic compound, only Compound 1. In this embodiment, Compound 1 may be included in the emission layer of the organic light-emitting device. In one or more embodiments, the organic layer may include, as the organometallic compound, Compound 1 and Compound 2. In this embodiment, Compound 1 and Compound 2 may exist in an identical layer (for example, Compound 1 and Compound 2 all may exist in an emission layer).

The first electrode may be an anode, which is a hole injection electrode, and the second electrode may be a cathode, which is an electron injection electrode; or the first electrode may be a cathode, which is an electron injection electrode, and the second electrode may be an anode, which is a hole injection electrode.

In one or more embodiments, in the organic light-emitting device, the first electrode is an anode, and the second electrode is a cathode, and the organic layer may further include a hole transport region located between the first electrode and the emission layer and an electron transport region located between the emission layer and the second electrode, and the hole transport region may include a hole injection layer, a hole transport layer, an electron blocking layer, a buffer layer, or a combination thereof, and the electron transport region may include a hole blocking layer, an electron transport layer, an electron injection layer, or a combination thereof.

The term “organic layer” used herein refers to a single layer and/or a plurality of layers located between the first electrode and the second electrode of the organic light-emitting device. The “organic layer” may include, in addition to an organic compound, an organometallic complex including metal.

FIGURE is a schematic cross-sectional view of an organic light-emitting device 10 according to one or more embodiments. Hereinafter, the structure of an organic light-emitting device and a method of manufacturing an organic light-emitting device according to one or more embodiments of the present disclosure will be described in connection with FIGURE. The organic light-emitting device 10 includes a first electrode 11, an organic layer 15, and a second electrode 19, which are sequentially stacked.

A substrate may be additionally located under the first electrode 11 or above the second electrode 19. For use as the substrate, any substrate that is used in organic light-emitting devices available in the art may be used, and the substrate may be a glass substrate or a transparent plastic substrate, each having excellent mechanical strength, thermal stability, transparency, surface smoothness, ease of handling, and water resistance.

In one or more embodiments, the first electrode 11 may be formed by depositing or sputtering a material for forming the first electrode 11 on the substrate. The first electrode 11 may be an anode. The material for forming the first electrode 11 may be selected from materials with a high work function to facilitate hole injection. The first electrode 11 may be a reflective electrode, a semi-transmissive electrode, or a transmissive electrode. The material for forming the first electrode 11 may be indium tin oxide (ITO), indium zinc oxide (IZO), tin oxide (SnO₂), or zinc oxide (ZnO). In one or more embodiments, the material for forming the first electrode 11 may be metal, such as magnesium (Mg), aluminum (Al), silver (Ag), aluminum-lithium (Al—Li), calcium (Ca), magnesium-indium (Mg—In), or magnesium-silver (Mg—Ag).

The first electrode 11 may have a single-layered structure or a multi-layered structure including two or more layers. For example, the first electrode 11 may have a three-layered structure of ITO/Ag/ITO, but the structure of the first electrode 11 is not limited thereto.

The organic layer 15 is located on the first electrode 11.

The organic layer 15 may include a hole transport region, an emission layer, and an electron transport region.

The hole transport region may be between the first electrode 11 and the emission layer.

The hole transport region may include a hole injection layer, a hole transport layer, an electron blocking layer, a buffer layer, or a combination thereof.

The hole transport region may include only either a hole injection layer or a hole transport layer. In one or more embodiments, the hole transport region may have a hole injection layer/hole transport layer structure or a hole injection layer/hole transport layer/electron blocking layer structure, wherein, for each structure, each layer is sequentially stacked in this stated order from the first electrode 11.

When the hole transport region includes a hole injection layer, the hole injection layer may be formed on the first electrode 11 by using one or more suitable methods, for example, vacuum deposition, spin coating, casting, and/or Langmuir-Blodgett (LB) deposition.

When a hole injection layer is formed by vacuum deposition, the deposition conditions may vary according to a material that is used to form the hole injection layer, and the structure and thermal characteristics of the hole injection layer. For example, the deposition conditions may include a deposition temperature of about 100° C. to about 500° C., a vacuum pressure of about 10⁻⁸ torr to about 10⁻³ torr, and a deposition rate of about 0.01 (angstrom per second) Å/sec to about 100 Å/sec. However, the deposition conditions are not limited thereto.

When the hole injection layer is formed using spin coating, coating conditions may vary according to the material used to form the hole injection layer, and the structure and thermal properties of the hole injection layer. For example, a coating speed may be from about 2,000 revolutions per minute (rpm) to about 5,000 rpm, and a temperature at which a heat treatment is performed to remove a solvent after coating may be from about 80° C. to about 200° C. However, the coating conditions are not limited thereto.

The conditions for forming the hole transport layer and the electron blocking layer may be the same as the conditions for forming the hole injection layer.

The hole transport region may include at least one selected from 4,4′,4″-tris(3-methylphenyl phenylamino)triphenylamine (m-MTDATA), 4,4,4″-tris(N,N-diphenylamino)triphenylamine (TDATA), 4,44″-tris{N-(2-naphthyl)-N-phenylamino}-triphenylamine (2-TNATA), N,N′-di(1-naphthyl)-N,N′-diphenylbenzidine (NPB), β-NPB, N,N′-bis(3-methylphenyl)-N,N′-diphenyl-[1,1-biphenyl]-4,4diamine (TPD), Spiro-TPD, Spiro-NPB, methylated NPB, 4,4′-cyclohexylidene bis[N,N-bis(4-methylphenyl)benzenamine] (TAPC), 4,4′-bis[N,N′-(3-tolyl)amino]-3,3′-dimethylbipheny (HMTPD), 4,4′,4″-tris(N-carbazolyl)triphenylamine (TCTA), polyaniline/dodecylbenzenesulfonic acid (PANI/DBSA), poly(3,4-ethylenedioxythiophene)/poly(4-styrenesulfonate) (PEDOT/PSS), polyaniline/camphor sulfonic acid (PANI/CSA), polyaniline/poly(4-styrenesulfonate) (PANI/PSS), a compound represented by Formula 201 below, and a compound represented by Formula 202 below:

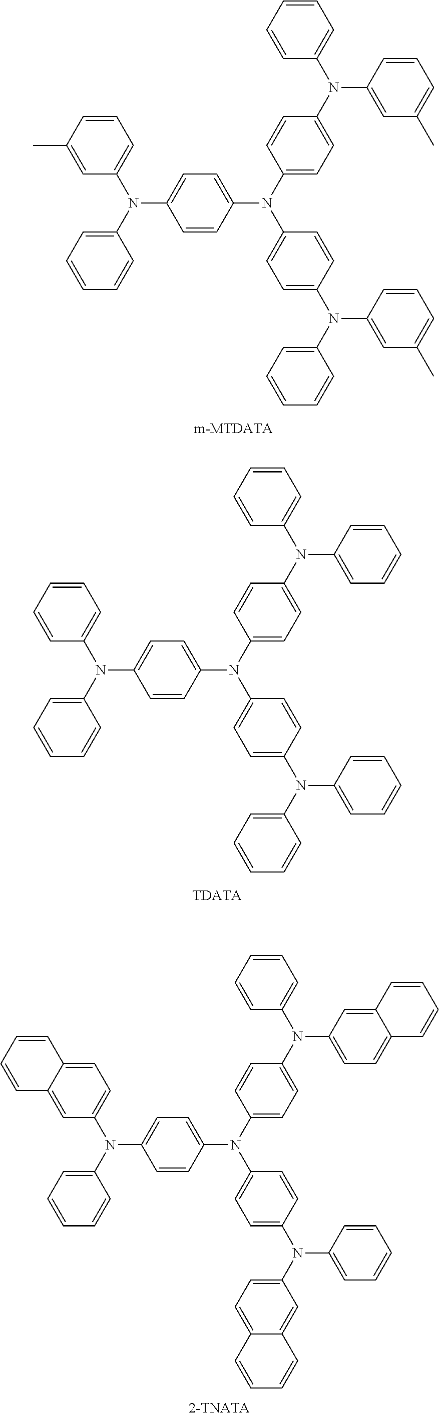

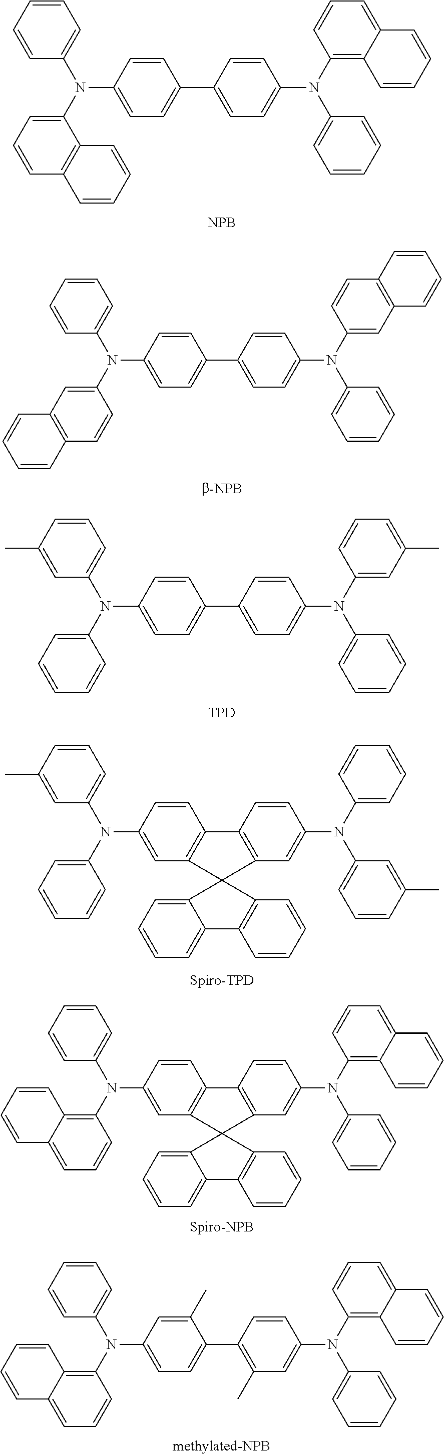

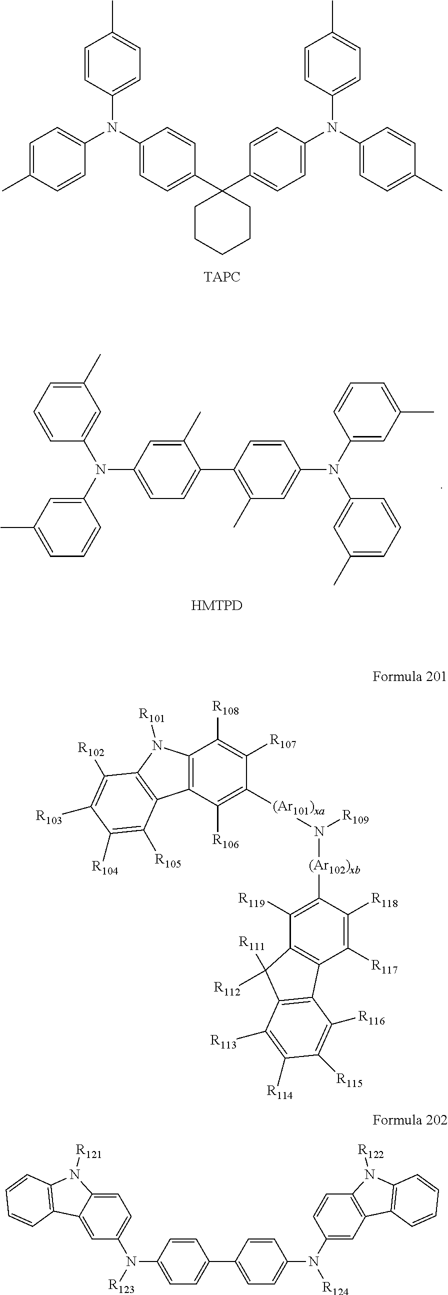

Ar₁₀₁ and Ar₁₀₂ in Formula 201 may each independently be:

a phenylene group, a pentalenylene group, an indenylene group, a naphthylene group, an azulenylene group, a heptalenylene group, an acenaphthylene group, a fluorenylene group, a phenalenylene group, a phenanthrenylene group, an anthracenylene group, a fluoranthenylene group, a triphenylenylene group, a pyrenylene group, a chrysenylenylene group, a naphthacenylene group, a picenylene group, a perylenylene group, or a pentacenylene group; or

a phenylene group, a pentalenylene group, an indenylene group, a naphthylene group, an azulenylene group, a heptalenylene group, an acenaphthylene group, a fluorenylene group, a phenalenylene group, a phenanthrenylene group, an anthracenylene group, a fluoranthenylene group, a triphenylenylene group, a pyrenylene group, a chrysenylenylene group, a naphthacenylene group, a picenylene group, a perylenylene group, or a pentacenylene group, each substituted with deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkoxy group, a C₁-C₆₀ alkylthio group, a C₃-C₁₀ cycloalkyl group, a C₃-C₁₀ cycloalkenyl group, a C₁₁-C₁₀ heterocycloalkyl group, a C₁-C₁₀ heterocycloalkenyl group, a C₆-C₆₀ aryl group, a C₇-C₆₀ alky aryl group, a C₇-C₆₀ aryl alkyl group, a C₆-C₆₀ aryloxy group, a C₆-C₆₀ arylthio group, a C₁-C₆₀ heteroaryl group, a C₂-C₆₀ alkyl heteroaryl group, a C₂-C₆₀ heteroaryl alkyl group, a C₁-C₆₀ heteroaryloxy group, a C₁-C₆₀ heteroarylthio group, a monovalent non-aromatic condensed polycyclic group, a monovalent non-aromatic condensed heteropolycyclic group, or a combination thereof.

xa and xb in Formula 201 may each independently be an integer from 0 to 5, or 0, 1, or 2. For example, xa may be 1 and xb may be 0, but xa and xb are not limited thereto.

R₁₀₁ to R₁₀₈, R₁₁₁ to R₁₁₉, and R₁₂₁ to R₁₂₄ in Formulae 201 and 202 may each independently be:

hydrogen, deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₁₀ alkyl group (for example, a methyl group, an ethyl group, a propyl group, a butyl group, pentyl group, a hexyl group, and the like) or a C₁-C₁₀ alkoxy group (for example, a methoxy group, an ethoxy group, a propoxy group, a butoxy group, a pentoxy group, and the like);

a C₁-C₁₀ alkyl group or a C₁-C₁₀ alkoxy group, each substituted with deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, or a combination thereof;

a phenyl group, a naphthyl group, an anthracenyl group, a fluorenyl group, or a pyrenyl group; or

a phenyl group, a naphthyl group, an anthracenyl group, a fluorenyl group, or a pyrenyl group, each substituted with deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₁₀ alkyl group, a C₁-C₁₀ alkoxy group, or a combination thereof, but embodiments of the present disclosure are not limited thereto.

R₁₀₉ in Formula 201 may be:

a phenyl group, a naphthyl group, an anthracenyl group, or a pyridinyl group; or

a phenyl group, a naphthyl group, an anthracenyl group, or a pyridinyl group, each substituted with deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₂₀ alkyl group, a C₁-C₂₀ alkoxy group, a phenyl group, a naphthyl group, an anthracenyl group, a pyridinyl group, or a combination thereof.

According to one or more embodiments, the compound represented by Formula 201 may be represented by Formula 201A below, but embodiments of the present disclosure are not limited thereto:

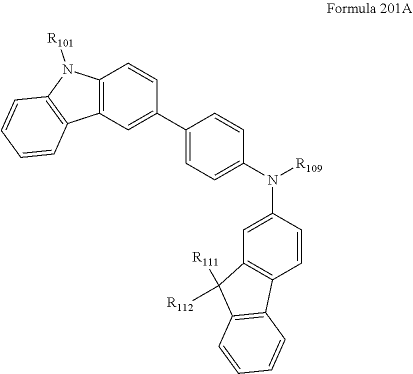

R₁₀₁, R₁₁₁, R₁₁₂, and R₁₀₉ in Formula 201A may be understood by referring to the description provided herein.

For example, the compound represented by Formula 201, and the compound represented by Formula 202 may include compounds HT1 to HT20 below, but are not limited thereto:

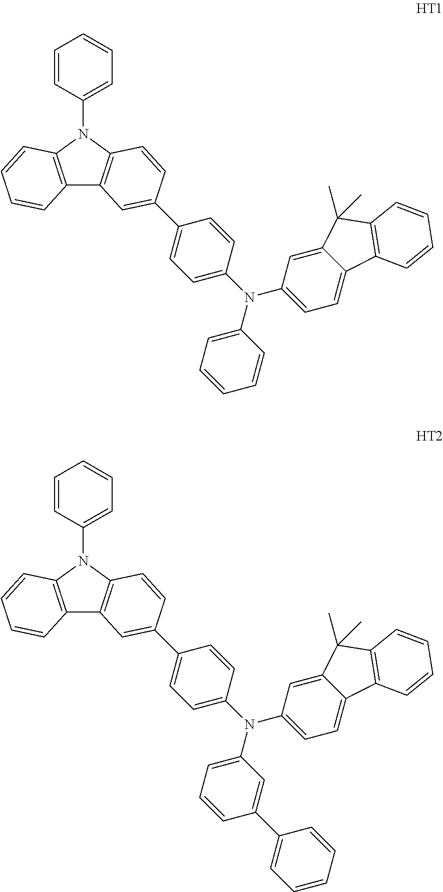 [SMILES: CC1(C)c2ccccc2-c2ccc(N(c3ccccc3)c3ccc(-c4ccc5c(c4)c4ccccc4n5-c4ccccc4)cc3)cc21 ; CC1(C)c2ccccc2-c2ccc(N(c3ccc(-c4ccc5c(c4)c4ccccc4n5-c4ccccc4)cc3)c3cccc(-c4ccccc4)c3)cc21]

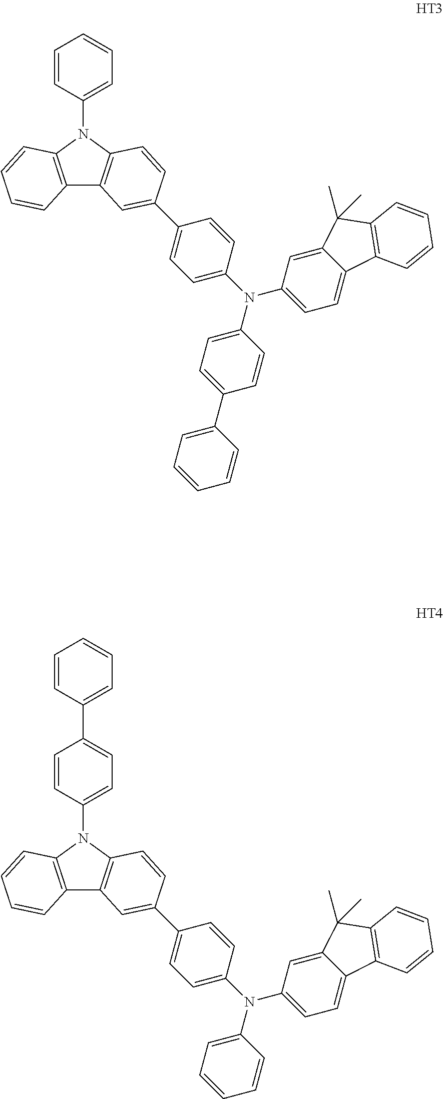 [SMILES: CC1(C)c2ccccc2-c2ccc(N(c3ccc(-c4ccccc4)cc3)c3ccc(-c4ccc5c(c4)c4ccccc4n5-c4ccccc4)cc3)cc21 ; CC1(C)c2ccccc2-c2ccc(N(c3ccccc3)c3ccc(-c4ccc5c(c4)c4ccccc4n5-c4ccc(-c5ccccc5)cc4)cc3)cc21]

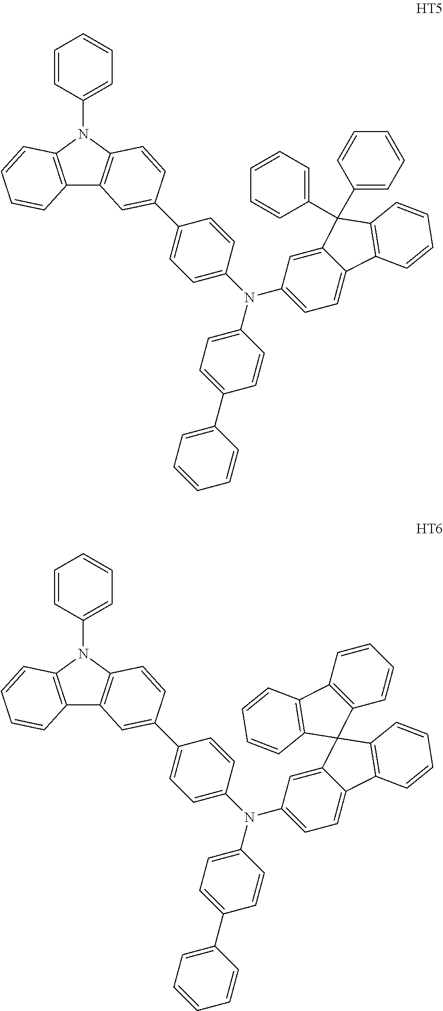 [SMILES: c1ccc(-c2ccc(N(c3ccc(-c4ccc5c(c4)c4ccccc4n5-c4ccccc4)cc3)c3ccc4c(c3)C(c3ccccc3)(c3ccccc3)c3ccccc3-4)cc2)cc1 ; c1ccc(-c2ccc(N(c3ccc(-c4ccc5c(c4)c4ccccc4n5-c4ccccc4)cc3)c3ccc4c(c3)C3(c5ccccc5-c5ccccc53)c3ccccc3-4)cc2)cc1]

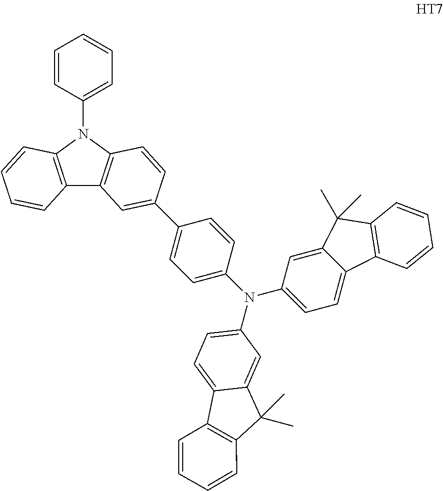 [SMILES: CC1(C)c2ccccc2-c2ccc(N(c3ccc(-c4ccc5c(c4)c4ccccc4n5-c4ccccc4)cc3)c3ccc4c(c3)C(C)(C)c3ccccc3-4)cc21]

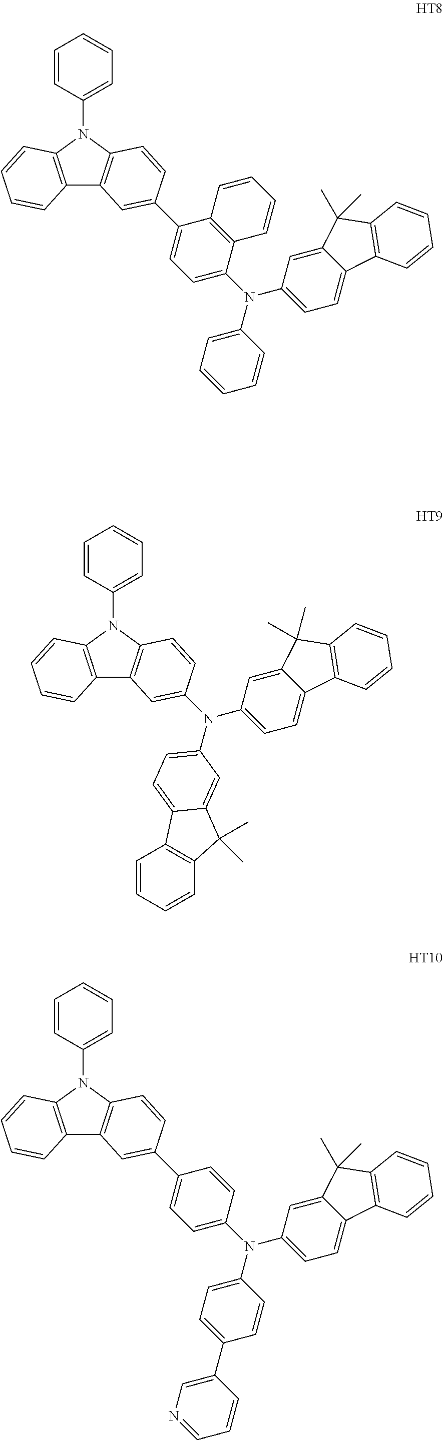 [SMILES: CC1(C)c2ccccc2-c2ccc(N(c3ccccc3)c3ccc(-c4ccc5c(c4)c4ccccc4n5-c4ccccc4)c4ccccc34)cc21 ; CC1(C)c2ccccc2-c2ccc(N(c3ccc4c(c3)C(C)(C)c3ccccc3-4)c3ccc4c(c3)c3ccccc3n4-c3ccccc3)cc21 ; CC1(C)c2ccccc2-c2ccc(N(c3ccc(-c4cccnc4)cc3)c3ccc(-c4ccc5c(c4)c4ccccc4n5-c4ccccc4)cc3)cc21]

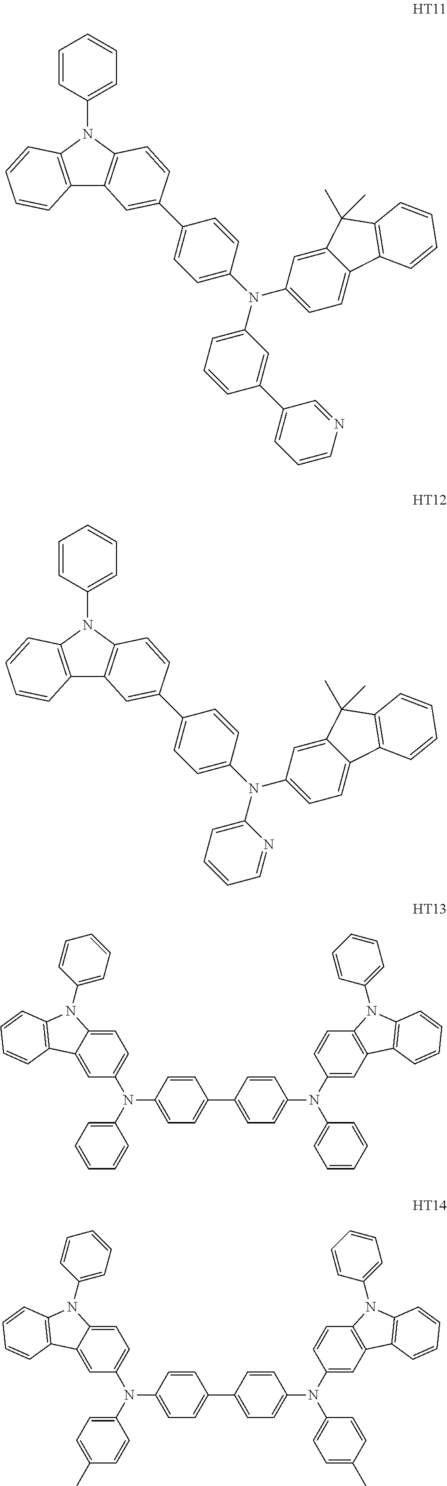 [SMILES: CC1(C)c2ccccc2-c2ccc(N(c3ccc(-c4ccc5c(c4)c4ccccc4n5-c4ccccc4)cc3)c3cccc(-c4cccnc4)c3)cc21 ; CC1(C)c2ccccc2-c2ccc(N(c3ccc(-c4ccc5c(c4)c4ccccc4n5-c4ccccc4)cc3)c3ccccn3)cc21 ; c1ccc(N(c2ccc(-c3ccc(N(c4ccccc4)c4ccc5c(c4)c4ccccc4n5-c4ccccc4)cc3)cc2)c2ccc3c(c2)c2ccccc2n3-c2ccccc2)cc1 ; Cc1ccc(N(c2ccc(-c3ccc(N(c4ccc(C)cc4)c4ccc5c(c4)c4ccccc4n5-c4ccccc4)cc3)cc2)c2ccc3c(c2)c2ccccc2n3-c2ccccc2)cc1]

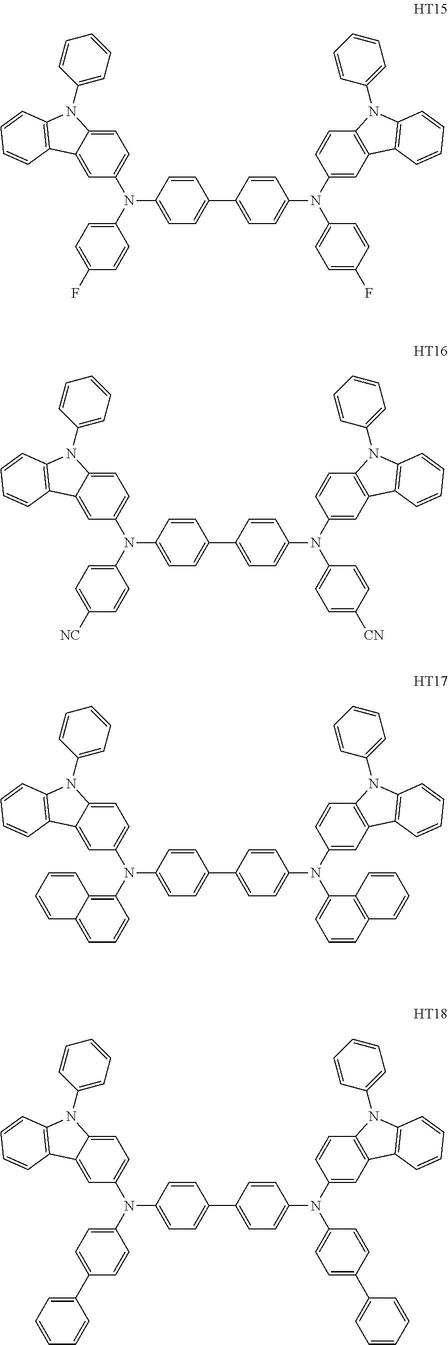 [SMILES: Fc1ccc(N(c2ccc(-c3ccc(N(c4ccc(F)cc4)c4ccc5c(c4)c4ccccc4n5-c4ccccc4)cc3)cc2)c2ccc3c(c2)c2ccccc2n3-c2ccccc2)cc1 ; N#Cc1ccc(N(c2ccc(-c3ccc(N(c4ccc(C#N)cc4)c4ccc5c(c4)c4ccccc4n5-c4ccccc4)cc3)cc2)c2ccc3c(c2)c2ccccc2n3-c2ccccc2)cc1 ; c1ccc(-n2c3ccccc3c3cc(N(c4ccc(-c5ccc(N(c6ccc7c(c6)c6ccccc6n7-c6ccccc6)c6cccc7ccccc67)cc5)cc4)c4cccc5ccccc45)ccc32)cc1 ; c1ccc(-c2ccc(N(c3ccc(-c4ccc(N(c5ccc(-c6ccccc6)cc5)c5ccc6c(c5)c5ccccc5n6-c5ccccc5)cc4)cc3)c3ccc4c(c3)c3ccccc3n4-c3ccccc3)cc2)cc1]

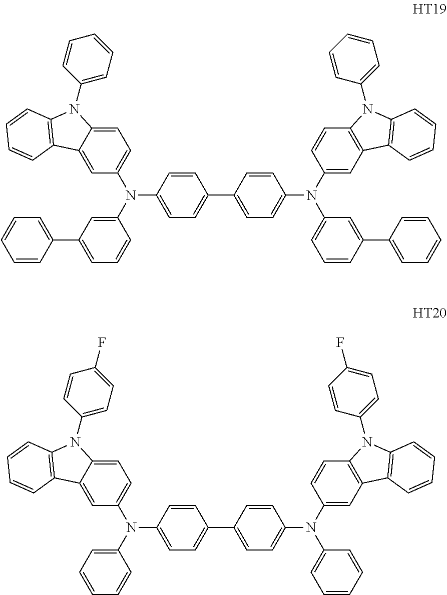 [SMILES: c1ccc(-c2cccc(N(c3ccc(-c4ccc(N(c5cccc(-c6ccccc6)c5)c5ccc6c(c5)c5ccccc5n6-c5ccccc5)cc4)cc3)c3ccc4c(c3)c3ccccc3n4-c3ccccc3)c2)cc1 ; Fc1ccc(-n2c3ccccc3c3cc(N(c4ccccc4)c4ccc(-c5ccc(N(c6ccccc6)c6ccc7c(c6)c6ccccc6n7-c6ccc(F)cc6)cc5)cc4)ccc32)cc1]

A thickness of the hole transport region may be in the range of about 100 Å to about 10,000 Å, for example, about 100 Å to about 1,000 Å. When the hole transport region includes at least one of a hole injection layer and a hole transport layer, a thickness of the hole injection layer may be in a range of about 100 Å to about 10,000 Å, for example, about 100 Å to about 1,000 Å, and a thickness of the hole transport layer may be in a range of about 50 Å to about 2,000 Å, for example, about 100 Å to about 1,500 Å. When the thicknesses of the hole transport region, the hole injection layer, and the hole transport layer are within these ranges, satisfactory hole transporting characteristics may be obtained without a substantial increase in driving voltage.

The hole transport region may further include, in addition to these materials, a charge-generation material for the improvement of conductive properties. The charge-generation material may be homogeneously or non-homogeneously dispersed in the hole transport region.

The charge-generation material may be, for example, a p-dopant. The p-dopant may be one selected from a quinone derivative, a metal oxide, and a cyano group-containing compound, but embodiments of the present disclosure are not limited thereto. Examples of the p-dopant include a quinone derivative, such as tetracyanoquinonedimethane (TCNQ) or 2,3,5,6-tetrafluoro-tetracyano-1,4-benzoquinonedimethane (F4-TCNQ); a metal oxide, such as a tungsten oxide or a molybdenum oxide; and a cyano group-containing compound, such as Compound HT-D1 below, but are not limited thereto.

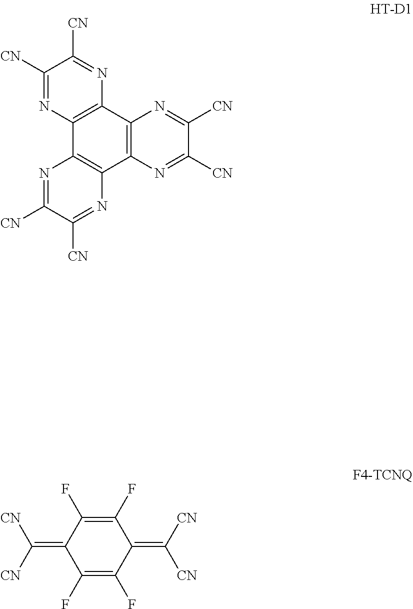

The hole transport region may further include a buffer layer.

The buffer layer may compensate for an optical resonance distance according to a wavelength of light emitted from the emission layer, and thus, efficiency of a formed organic light-emitting device may be improved.

Then, an emission layer may be formed on the hole transport region by vacuum deposition, spin coating, casting, LB deposition, and the like. When the emission layer is formed by vacuum deposition or spin coating, the deposition or coating conditions may be similar to those applied in forming the hole injection layer although the deposition or coating conditions may vary according to a compound that is used to form the hole transport layer.

Meanwhile, when the hole transport region includes an electron blocking layer, a material for the electron blocking layer may be selected from materials for the hole transport region described above and materials for a host to be explained later. However, the material for the electron blocking layer is not limited thereto. For example, when the hole transport region includes an electron blocking layer, a material for the electron blocking layer may be mCP, which will be explained later.

The emission layer may include a host and a dopant, and the dopant may include the organometallic compound represented by Formula 1.

The host may include at least one selected from 1,3,5-tris(N-phenylbenzimidazole-2-yl)benzene (TPBi), 3-tert-butyl-9,10-di(naphth-2-yl)anthracene (TBADN), 9,10-di(naphthalene-2-yl)anthracene (ADN) (also referred to as “DNA”), 4,4′-bis(N-carbazolyl)-1,1′-biphenyl (CBP), 4,4′-bis(9-carbazolyl)-2,2′-dimethyl-biphenyl (CDBP), TCP, mCP, Compound H50, and Compound H51:

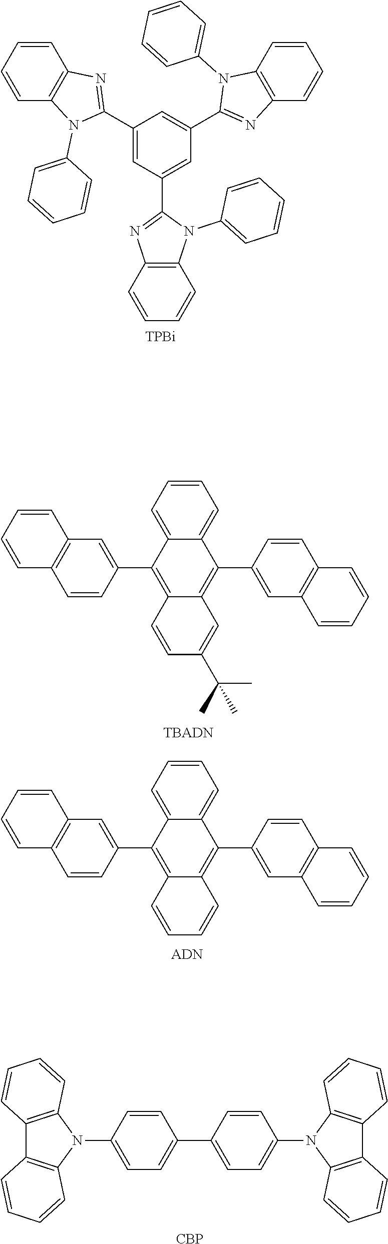

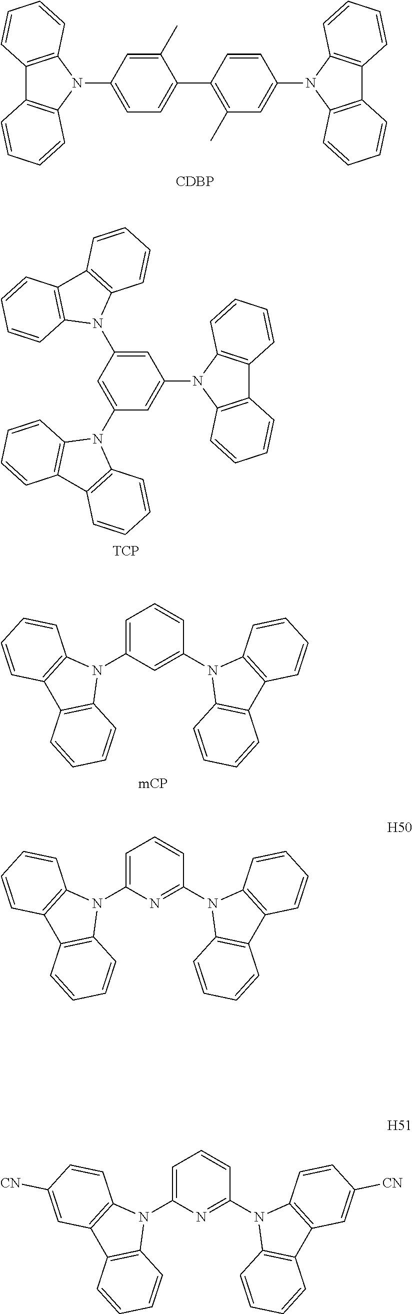

In one or more embodiments, the host may further include a compound represented by Formula 301:

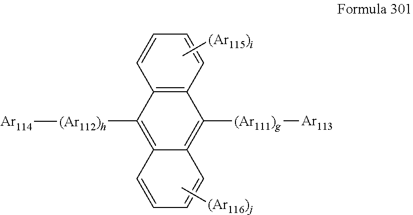

Ar₁₁₁ and Ar₁₁₂ in Formula 301 may each independently be:

a phenylene group, a naphthylene group, a phenanthrenylene group, or a pyrenylene group; or

a phenylene group, a naphthylene group, a phenanthrenylene group, or a pyrenylene group, each substituted with a phenyl group, a naphthyl group, an anthracenyl group, or a combination thereof.

Ar₁₁₃ to Ar₁₁₆ in Formula 301 may each independently be:

a C₁-C₁₀ alkyl group, a phenyl group, a naphthyl group, a phenanthrenyl group, or a pyrenyl group; or

a phenyl group, a naphthyl group, a phenanthrenyl group, or a pyrenyl group, each substituted with a phenyl group, a naphthyl group, an anthracenyl group, or a combination thereof.

g, h, i, and j in Formula 301 may each independently be an integer from 0 to 4, and may be, for example, 0, 1, or 2.

Ar₁₁₃ and Ar₁₁₆ in Formula 301 may each independently be:

a C₁-C₁₀ alkyl group which is substituted with a phenyl group, a naphthyl group, an anthracenyl group, or a combination thereof;

a phenyl group, a naphthyl group, an anthracenyl group, a pyrenyl group, a phenanthrenyl group, or a fluorenyl group;

a phenyl group, a naphthyl group, an anthracenyl group, a pyrenyl group, a phenanthrenyl group, or a fluorenyl group, each substituted with deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkoxy group, a phenyl group, a naphthyl group, an anthracenyl group, a pyrenyl group, a phenanthrenyl group, a fluorenyl group, or a combination thereof, or

a group represented by the formula:

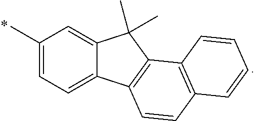 [SMILES: Cc1ccc2c(c1)C(C)(C)c1c-2ccc2ccccc12]

In one or more embodiments, the host may include a compound represented by Formula 302 below:

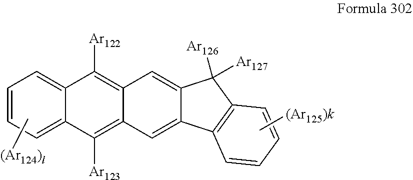

Ar₁₂₂ to Ar₁₂₅ in Formula 302 are the same as described in detail in connection with Ar₁₁₃ in Formula 301.

Ar₁₂₆ and Ar₁₂₇ in Formula 302 may each independently be a C₁-C₁₀ alkyl group (for example, a methyl group, an ethyl group, or a propyl group).

k and l in Formula 302 may each independently be an integer from 0 to 4. For example, k and l may be 0, 1, or 2.

When the organic light-emitting device is a full-color organic light-emitting device, the emission layer may be patterned into a red emission layer, a green emission layer, and a blue emission layer. In one or more embodiments, due to a stacked structure including a red emission layer, a green emission layer, and/or a blue emission layer, the emission layer may emit white light.

When the emission layer includes a host and a dopant, an amount of the dopant may be in a range of about 0.01 parts by weight to about 15 parts by weight based on 100 parts by weight of the host, but embodiments of the present disclosure are not limited thereto.

A thickness of the emission layer may be in a range of about 100 Å to about 1,000 Å, for example, about 200 Å to about 600 Å. When the thickness of the emission layer is within these ranges, excellent light-emission characteristics may be obtained without a substantial increase in driving voltage.

Then, an electron transport region may be located on the emission layer.

The electron transport region may include a hole blocking layer, an electron transport layer, an electron injection layer, or a combination thereof.

For example, the electron transport region may have a hole blocking layer/electron transport layer/electron injection layer structure or an electron transport layer/electron injection layer structure, and the structure of the electron transport region is not limited thereto. The electron transport layer may have a single-layered structure or a multi-layered structure including two or more different materials.

Conditions for forming the hole blocking layer, the electron transport layer, and the electron injection layer which constitute the electron transport region may be understood by referring to the conditions for forming the hole injection layer.

When the electron transport region includes a hole blocking layer, the hole blocking layer may include, for example, at least one of 2,9-dimethyl-4,7-diphenyl-1,10-phenanthroline (BCP), bis(2-methyl-8-quinolinolato-N1,08)-(1,1′-biphenyl-4-olato)aluminum (Bphen), and BAlq but embodiments of the present disclosure are not limited thereto.

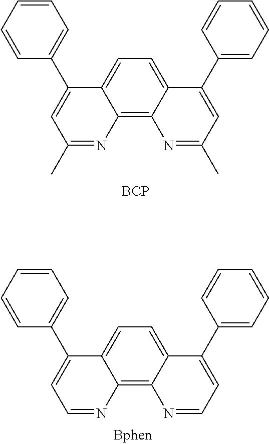

A thickness of the hole blocking layer may be in a range of about 20 Å to about 1,000 Å, for example, about 30 Å to about 300 Å. When the thickness of the hole blocking layer is within these ranges, excellent hole blocking characteristics can be obtained without a substantial increase in driving voltage.

The electron transport layer may further include at least one selected from BCP, Bphen, tris(8-hydroxyquinolinato)aluminum (Alq₃), BAlq, 3-(4-biphenylyl)-4-phenyl-5-tert-butylphenyl-1,2,4-triazole (TAZ), and 4-(naphthalen-1-yl)-3,5-diphenyl-4H-1,2,4-triazole (NTAZ).

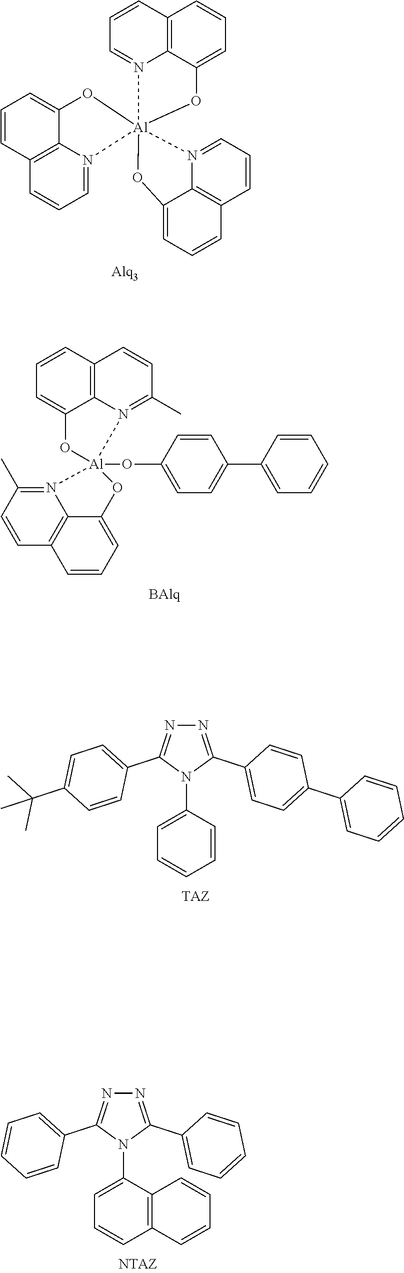

In one or more embodiments, the electron transport layer may include at least one of ET1 to ET25, but are not limited thereto:

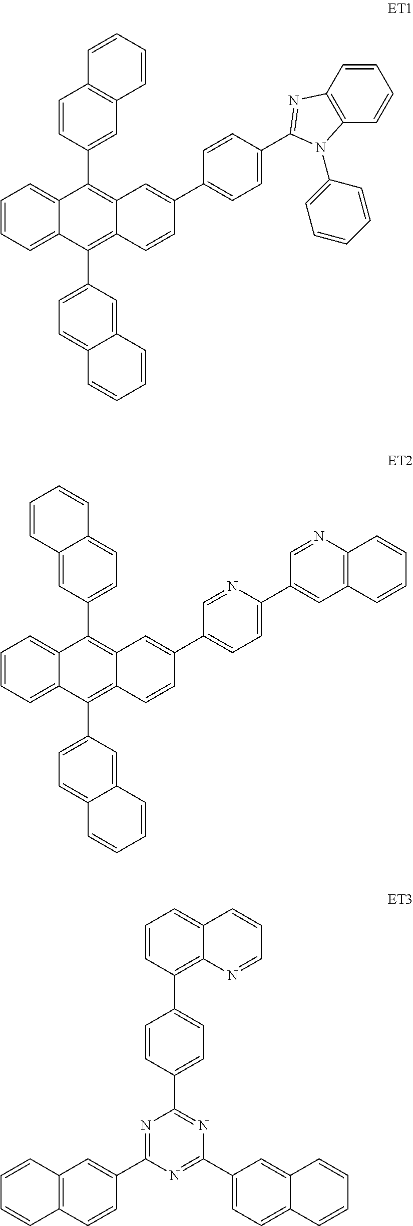 [SMILES: c1ccc(-n2c(-c3ccc(-c4ccc5c(-c6ccc7ccccc7c6)c6ccccc6c(-c6ccc7ccccc7c6)c5c4)cc3)nc3ccccc32)cc1 ; c1ccc2cc(-c3c4ccccc4c(-c4ccc5ccccc5c4)c4cc(-c5ccc(-c6cnc7ccccc7c6)nc5)ccc34)ccc2c1 ; c1ccc2cc(-c3nc(-c4ccc(-c5cccc6cccnc56)cc4)nc(-c4ccc5ccccc5c4)n3)ccc2c1]

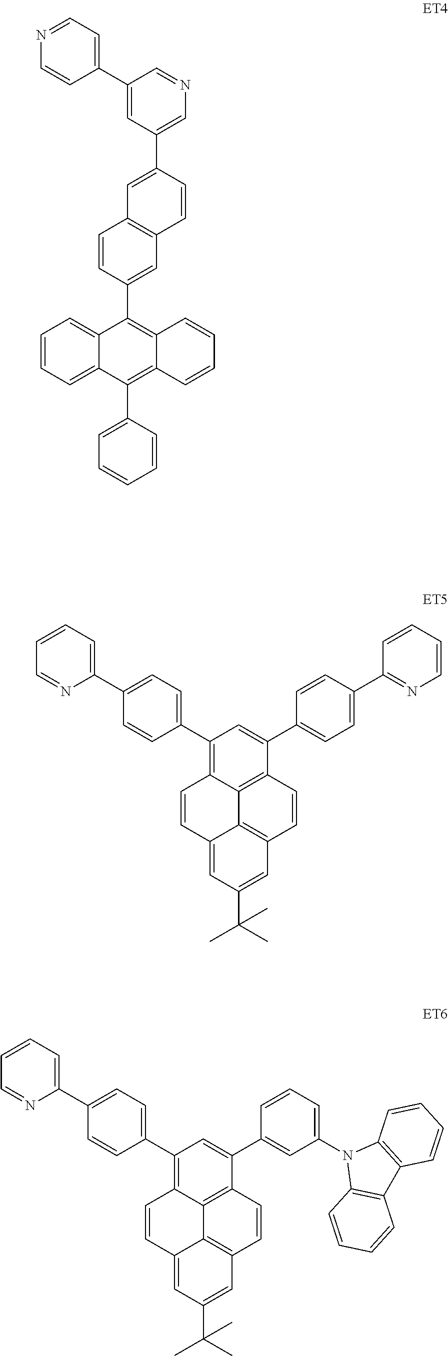 [SMILES: c1ccc(-c2c3ccccc3c(-c3ccc4cc(-c5cncc(-c6ccncc6)c5)ccc4c3)c3ccccc23)cc1 ; CC(C)(C)c1cc2ccc3c(-c4ccc(-c5ccccn5)cc4)cc(-c4ccc(-c5ccccn5)cc4)c4ccc(c1)c2c34 ; CC(C)(C)c1cc2ccc3c(-c4ccc(-c5ccccn5)cc4)cc(-c4cccc(-n5c6ccccc6c6ccccc65)c4)c4ccc(c1)c2c34]

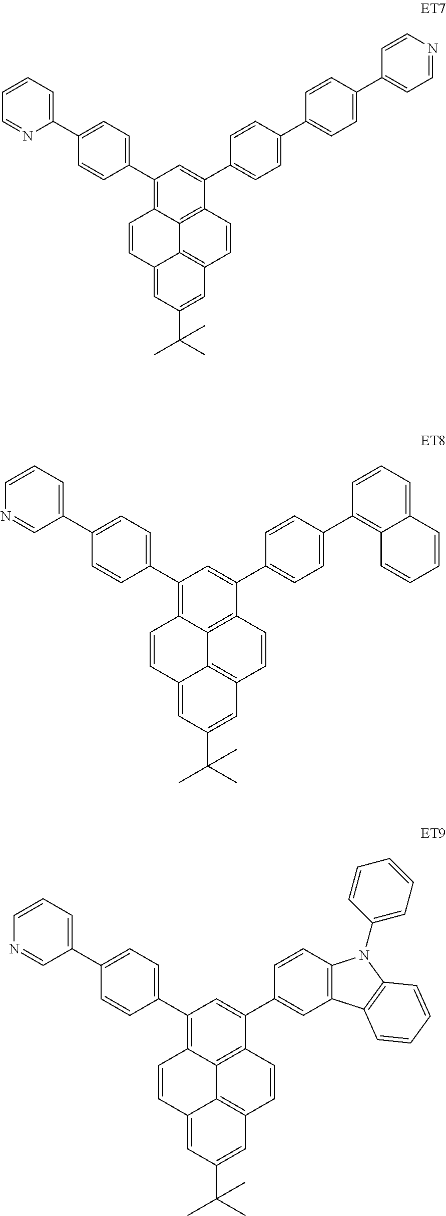 [SMILES: CC(C)(C)c1cc2ccc3c(-c4ccc(-c5ccc(-c6ccncc6)cc5)cc4)cc(-c4ccc(-c5ccccn5)cc4)c4ccc(c1)c2c34 ; CC(C)(C)c1cc2ccc3c(-c4ccc(-c5cccnc5)cc4)cc(-c4ccc(-c5cccc6ccccc56)cc4)c4ccc(c1)c2c34 ; CC(C)(C)c1cc2ccc3c(-c4ccc(-c5cccnc5)cc4)cc(-c4ccc5c(c4)c4ccccc4n5-c4ccccc4)c4ccc(c1)c2c34]

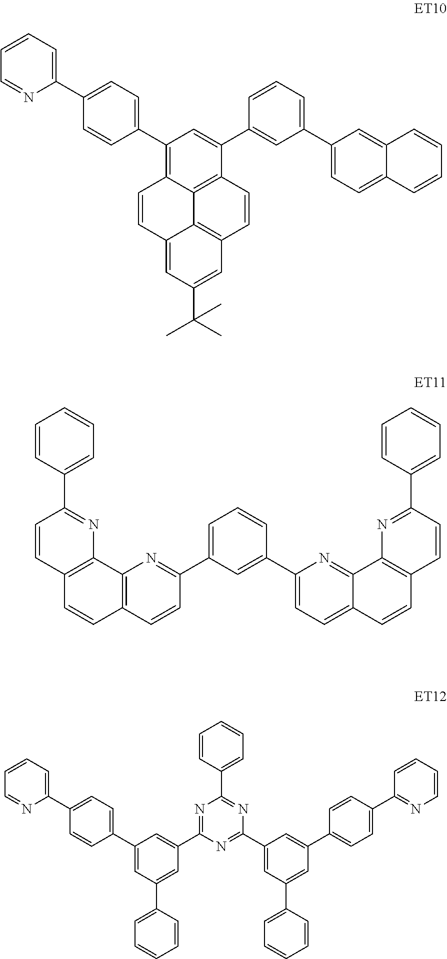 [SMILES: CC(C)(C)c1cc2ccc3c(-c4ccc(-c5ccccn5)cc4)cc(-c4cccc(-c5ccc6ccccc6c5)c4)c4ccc(c1)c2c34 ; c1ccc(-c2ccc3ccc4ccc(-c5cccc(-c6ccc7ccc8ccc(-c9ccccc9)nc8c7n6)c5)nc4c3n2)cc1 ; c1ccc(-c2cc(-c3ccc(-c4ccccn4)cc3)cc(-c3nc(-c4ccccc4)nc(-c4cc(-c5ccccc5)cc(-c5ccc(-c6ccccn6)cc5)c4)n3)c2)cc1]

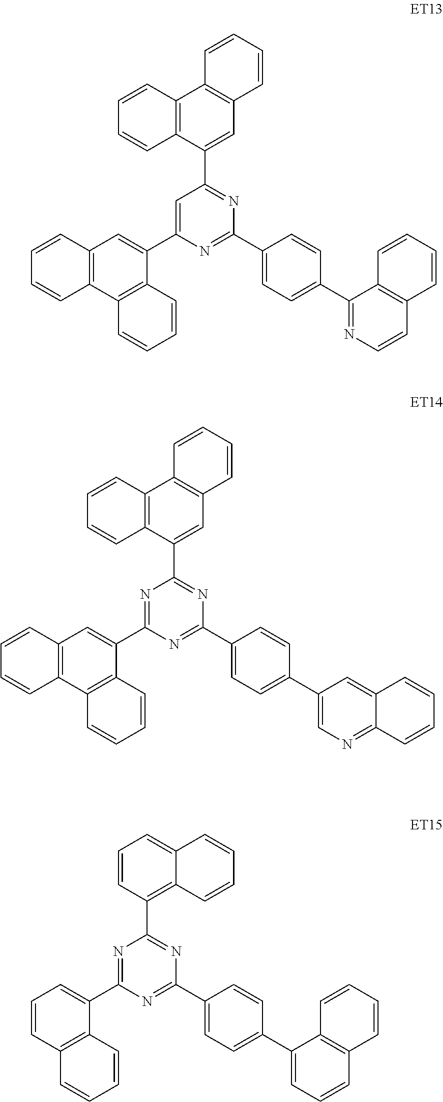 [SMILES: c1ccc2c(-c3ccc(-c4nc(-c5cc6ccccc6c6ccccc56)cc(-c5cc6ccccc6c6ccccc56)n4)cc3)nccc2c1 ; c1ccc2ncc(-c3ccc(-c4nc(-c5cc6ccccc6c6ccccc56)nc(-c5cc6ccccc6c6ccccc56)n4)cc3)cc2c1 ; c1ccc2c(-c3ccc(-c4nc(-c5cccc6ccccc56)nc(-c5cccc6ccccc56)n4)cc3)cccc2c1]

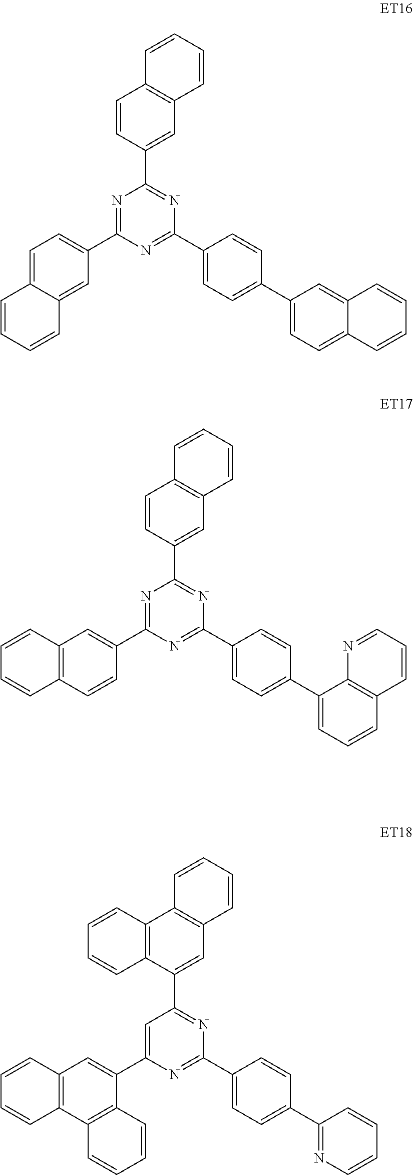 [SMILES: c1ccc2cc(-c3ccc(-c4nc(-c5ccc6ccccc6c5)nc(-c5ccc6ccccc6c5)n4)cc3)ccc2c1 ; c1ccc2cc(-c3nc(-c4ccc(-c5cccc6cccnc56)cc4)nc(-c4ccc5ccccc5c4)n3)ccc2c1 ; c1ccc(-c2ccc(-c3nc(-c4cc5ccccc5c5ccccc45)cc(-c4cc5ccccc5c5ccccc45)n3)cc2)nc1]

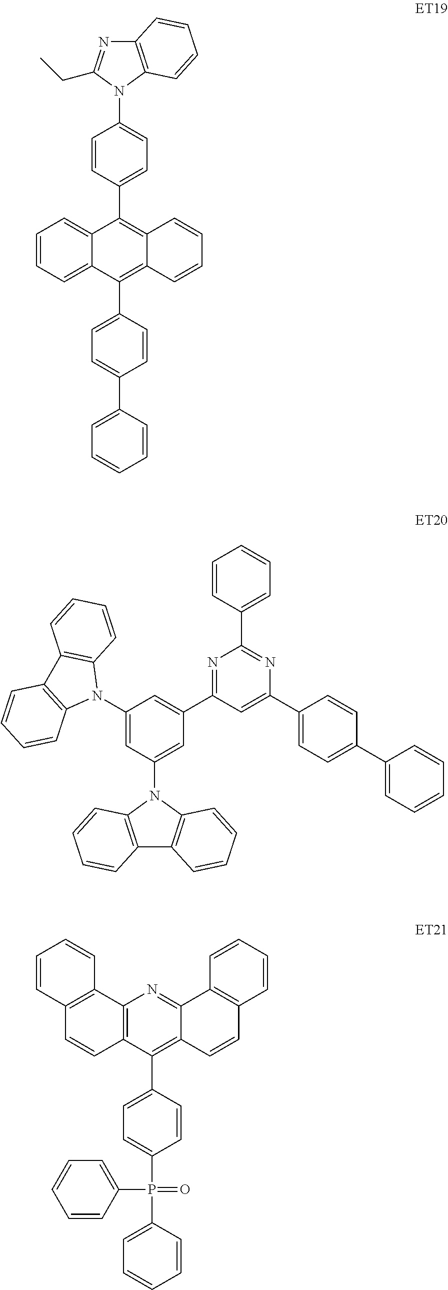 [SMILES: CCc1nc2ccccc2n1-c1ccc(-c2c3ccccc3c(-c3ccc(-c4ccccc4)cc3)c3ccccc23)cc1 ; c1ccc(-c2ccc(-c3cc(-c4cc(-n5c6ccccc6c6ccccc65)cc(-n5c6ccccc6c6ccccc65)c4)nc(-c4ccccc4)n3)cc2)cc1 ; O=P(c1ccccc1)(c1ccccc1)c1ccc(-c2c3ccc4ccccc4c3nc3c2ccc2ccccc23)cc1]

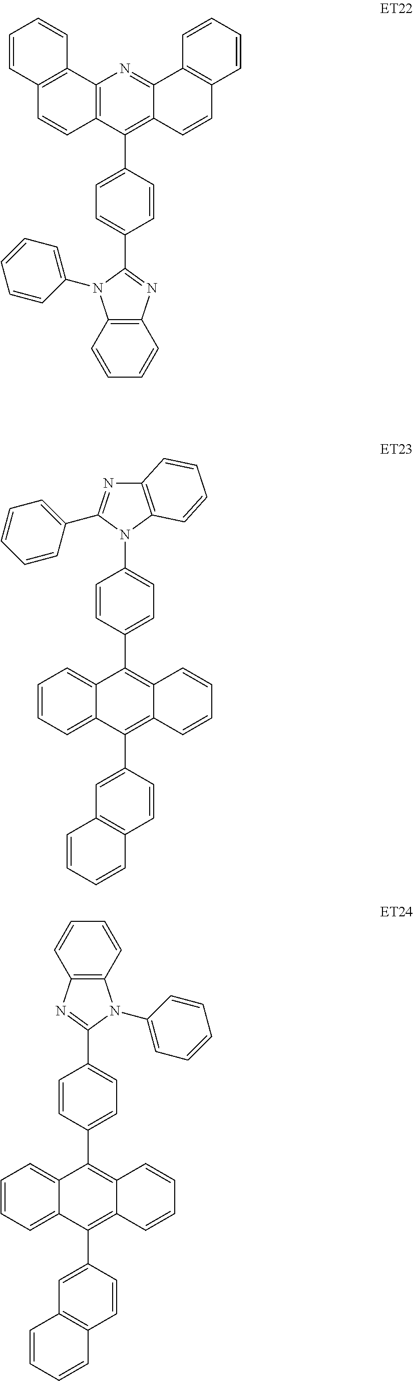

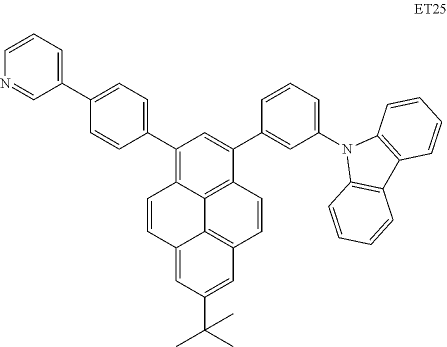 [SMILES: CC(C)(C)c1cc2ccc3c(-c4ccc(-c5cccnc5)cc4)cc(-c4cccc(-n5c6ccccc6c6ccccc65)c4)c4ccc(c1)c2c34]

A thickness of the electron transport layer may be in the range of about 100 Å to about 1,000 Å, for example, about 150 Å to about 500 Å. When the thickness of the electron transport layer is within the range described above, the electron transport layer may have satisfactory electron transporting characteristics without a substantial increase in driving voltage.

The electron transport layer may include a metal-containing material in addition to the material as described above.

The metal-containing material may include a Li complex. The Li complex may include, for example, Compound ET-D1 (lithium quinolate, LiQ) or ET-D2:

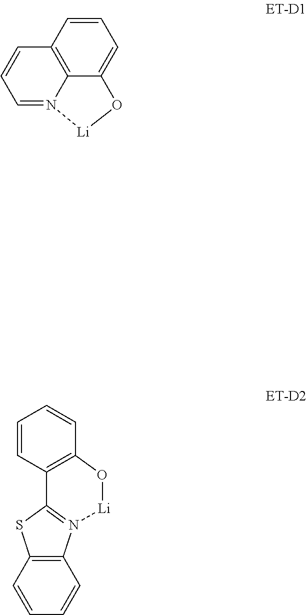

The electron transport region may include an electron injection layer (EIL) that promotes the flow of electrons from the second electrode 19 thereinto.

The electron injection layer may include at least one selected from LiF, NaCl, CsF, Li₂O, and BaO.

A thickness of the electron injection layer may be in a range of about 1 Å to about 100 Å, and, for example, about 3 Å to about 90 Å. When the thickness of the electron injection layer is within the range described above, the electron injection layer may have satisfactory electron injection characteristics without a substantial increase in driving voltage.

The second electrode 19 is located on the organic layer 15. The second electrode 19 may be a cathode. A material for forming the second electrode 19 may be metal, an alloy, an electrically conductive compound, or a combination thereof, which have a relatively low work function. For example, lithium (Li), magnesium (Mg), aluminum (Al), silver (Ag), aluminum-lithium (Al—Li), calcium (Ca), magnesium-indium (Mg—In), or magnesium-silver (Mg—Ag) may be used as the material for forming the second electrode 19. In one or more embodiments, to manufacture a top-emission type light-emitting device, a transmissive electrode formed using ITO or IZO may be used as the second electrode 19.

Hereinbefore, the organic light-emitting device has been described with reference to FIGURE, but embodiments of the present disclosure are not limited thereto.

Another aspect provides a diagnostic composition including at least one organometallic compound represented by Formula 1.

The organometallic compound represented by Formula 1 provides high luminescent efficiency. Accordingly, a diagnostic composition including the organometallic compound may have high diagnostic efficiency.

The diagnostic composition may be used in various applications including a diagnosis kit, a diagnosis reagent, a biosensor, and a biomarker.

The term “C₁-C₆₀ alkyl group” as used herein refers to a linear or branched saturated aliphatic hydrocarbon monovalent group having 1 to 60 carbon atoms, and non-limiting examples thereof include a methyl group, an ethyl group, a propyl group, an isobutyl group, a sec-butyl group, a tert-butyl group, a pentyl group, an isoamyl group, and a hexyl group. The term “C₁-C₆₀ alkylene group” as used herein refers to a divalent group having the same structure as the C₁-C₆₀ alkyl group.

The term “C₁-C₆₀ alkoxy group” used herein refers to a monovalent group represented by —OA₁₀₁ (wherein A₁₀₁ is the C₁-C₆₀ alkyl group), and examples thereof include a methoxy group, an ethoxy group, and an isopropyloxy group. The term “C₁-C₆₀ alkylthio group” as used herein is represented by —SA₁₀₄ (wherein A₁₀₄ is the C₁-C₆₀ alkyl group).

The term “C₂-C₆ alkenyl group” as used herein refers to a hydrocarbon group formed by substituting at least one carbon-carbon double bond in the middle or at the terminus of the C₂-C₆₀ alkyl group, and examples thereof include an ethenyl group, a propenyl group, and a butenyl group. The term “C₂-C₆₀ alkenylene group” as used herein refers to a divalent group having the same structure as the C₂-C₆₀ alkenyl group.

The term “C₂-C₆ alkynyl group” as used herein refers to a hydrocarbon group formed by substituting at least one carbon-carbon triple bond in the middle or at the terminus of the C₂-C₆₀ alkyl group, and examples thereof include an ethynyl group, and a propynyl group. The term “C₂-C₆₀ alkynylene group” as used herein refers to a divalent group having the same structure as the C₂-C₆₀ alkynyl group.

The term “C₃-C₁₀ cycloalkyl group” as used herein refers to a monovalent saturated hydrocarbon monocyclic group having 3 to 10 carbon atoms, and examples thereof include a cyclopropyl group, a cyclobutyl group, a cyclopentyl group, a cyclohexyl group, and a cycloheptyl group. The term “C₃-C₁₀ cycloalkylene group” as used herein refers to a divalent group having the same structure as the C₃-C₁₀ cycloalkyl group.

The term “C₁-C₁₀ heterocycloalkyl group” as used herein refers to a monovalent saturated monocyclic group having at least one heteroatom selected from N, O, P, Si, and S as a ring-forming atom and 1 to 10 carbon atoms, and non-limiting examples thereof include a tetrahydrofuranyl group, and a tetrahydrothiophenyl group. The term “C₁-C₁₀ heterocycloalkylene group” as used herein refers to a divalent group having the same structure as the C₁-C₁₀ heterocycloalkyl group.

The term “C₃-C₁₀ cycloalkenyl group” as used herein refers to a monovalent monocyclic group that has 3 to 10 carbon atoms and at least one carbon-carbon double bond in the ring thereof and no aromaticity, and non-limiting examples thereof include a cyclopentenyl group, a cyclohexenyl group, and a cycloheptenyl group. The term “C₃-C₁₀ cycloalkenylene group” as used herein refers to a divalent group having the same structure as the C₃-C₁₀ cycloalkenyl group.

The term “C₂-C₁₀ heterocycloalkenyl group” as used herein refers to a monovalent monocyclic group that has at least one hetero atom selected from N, O, P, Si, and S as a ring-forming atom, 2 to 10 carbon atoms, and at least one carbon-carbon double bond in its ring. Examples of the C₁-C₁₀ heterocycloalkenyl group are a 2,3-dihydrofuranyl group, and a 2,3-dihydrothiophenyl group. The term “C₂-C₁₀ heterocycloalkenylene group” as used herein refers to a divalent group having the same structure as the C₂-C₁₀ heterocycloalkenyl group.

The term “C₆-C₆₀ aryl group” as used herein refers to a monovalent group having a carbocyclic aromatic system having 6 to 60 carbon atoms, and the term “C₆-C₆₀ arylene group” as used herein refers to a divalent group having a carbocyclic aromatic system having 6 to 60 carbon atoms. Examples of the C₆-C₆₀ aryl group include a phenyl group, a naphthyl group, an anthracenyl group, a phenanthrenyl group, a pyrenyl group, and a chrysenyl group. When the C₆-C₆₀ aryl group and the C₆-C₆₀ arylene group each include two or more rings, the rings may be fused to each other.

The term “C₇-C₆₀ alkyl aryl group” used herein refers to a C₆-C₆₀ aryl group substituted with at least one C₁-C₆₀ alkyl group. The term “C₇-C₆₀ aryl alkyl group” as used herein refers to a C₁-C₆₀ alkyl group substituted with at least one C₆-C₆₀ aryl group.

The term “C₁-C₆₀ heteroaryl group” as used herein refers to a monovalent group having a cyclic aromatic system that has at least one heteroatom selected from N, O, P, Si, and S as a ring-forming atom, and 1 to 60 carbon atoms. The term “C₁-C₆₀ heteroarylene group” as used herein refers to a divalent group having a carbocyclic aromatic system that has at least one heteroatom selected from N, O, P, and S as a ring-forming atom, and 1 to 60 carbon atoms. Examples of the C₁-C₆₀ heteroaryl group include a pyridinyl group, a pyrimidinyl group, a pyrazinyl group, a pyridazinyl group, a triazinyl group, a quinolinyl group, and an isoquinolinyl group. When the C₁-C₆₀ heteroaryl group and the C₆-C₆₀ heteroarylene group each include two or more rings, the rings may be fused to each other.

The term “C₂-C₆₀ alkyl heteroaryl group” as used herein refers to a C₁-C₆₀ heteroaryl group substituted with at least one C₁-C₆₀ alkyl group. The term “C₇-C₆₀ heteroaryl alkyl group” as used herein refers to a C₁-C₆₀ alkyl group substituted with at least one C₁-C₆₀ heteroaryl group.

The term “C₆-C₆₀ aryloxy group” as used herein indicates —OA₁₀₂ (wherein A₁₀₂ is the C₆-C₆₀ aryl group), and the term “C₆-C₆₀ arylthio group” as used herein indicates —SA₁₀₃ (wherein A₁₀₃ is the C₆-C₆₀ aryl group).

The term “C₁-C₆ heteroaryloxy group” as used herein indicates —OA₁₀₄ (wherein A₁₀₄ is a C₁-C₆₀ heteroaryl group), and the C₁-C₆₀ heteroarylthio group(arylthio) indicates —SA₁₀ₛ(wherein A₁₀ₛ is the C₁-C₆₀ heteroaryl group).

The term “monovalent non-aromatic condensed polycyclic group” as used herein refers to a monovalent group (for example, having 8 to 60 carbon atoms) having two or more rings condensed to each other, only carbon atoms as ring-forming atoms, and no aromaticity in its entire molecular structure. Examples of the monovalent non-aromatic condensed polycyclic group include a fluorenyl group. The term “divalent non-aromatic condensed polycyclic group” as used herein refers to a divalent group having the same structure as a monovalent non-aromatic condensed polycyclic group.

The term “monovalent non-aromatic condensed heteropolycyclic group” as used herein refers to a monovalent group (for example, having 2 to 60 carbon atoms) having two or more rings condensed to each other, a heteroatom selected from N, O, P, Si, and S, other than carbon atoms, as a ring-forming atom, and no aromaticity in its entire molecular structure. Examples of the monovalent non-aromatic condensed heteropolycyclic group include a carbazolyl group. The term “divalent non-aromatic condensed heteropolycyclic group” as used herein refers to a divalent group having the same structure as a monovalent non-aromatic condensed heteropolycyclic group.

The term “C₅-C₃₀ carbocyclic group” as used herein refers to a saturated or unsaturated cyclic group having, as a ring-forming atom, 5 to 30 carbon atoms only. The C₅-C₃₀ carbocyclic group may be a monocyclic group or a polycyclic group.

The term “C₁-C₃₀ heterocyclic group” as used herein refers to a saturated or unsaturated cyclic group having, as a ring-forming atom, at least one heteroatom selected from N, O, Si, P, and S other than 1 to 30 carbon atoms. The C₁-C₃₀ heterocyclic group may be a monocyclic group or a polycyclic group.

At least one substituent of the substituted C₅-C₃₀ carbocyclic group, the substituted C₁-C₃₀ heterocyclic group, the substituted C₁-C₆₀ alkyl group, the substituted C₂-C₆₀ alkenyl group, the substituted C₂-C₆₀ alkynyl group, the substituted C₁-C₆₀ alkoxy group, the substituted C₁-C₆₀ alkylthio group, the substituted C₃-C₁₀ cycloalkyl group, the substituted C₁-C₁₀ heterocycloalkyl group, the substituted C₃-C₁₀ cycloalkenyl group, the substituted C₁-C₁₀ heterocycloalkenyl group, the substituted C₆-C₆₀ aryl group, the substituted C₇-C₆₀ alkyl aryl group, the substituted C₇-C₆₀ aryl alkyl group, the substituted C₆-C₆₀ aryloxy group, the substituted C₆-C₆₀ arylthio group, the substituted C₁-C₆₀ heteroaryl group, the substituted C₂-C₆₀ alkyl heteroaryl group, the substituted C₂-C₆₀ heteroaryl alkyl group, the substituted C₁-C₆₀ heteroaryloxy group, the substituted C₁-C₆₀ heteroarylthio group, the substituted monovalent non-aromatic condensed polycyclic group, and the substituted monovalent non-aromatic condensed heteropolycyclic group may be:

deuterium, —F, —Cl, —Br, —I, —CD₃, —CD₂H, —CDH₂, —CF₃, —CF₂H, —CFH₂, a hydroxyl group, a cyano group, a nitro group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, or a C₁-C₆₀ alkoxy group;

a C₁-C₆₀ alkyl group, a C₂-C₆₀ alkenyl group, a C₂-C₆₀ alkynyl group, or a C₁-C₆₀ alkoxy group, each substituted with deuterium, —F, —Cl, —Br, —I, —CD₃, —CD₂H, —CDH₂, —CF₃, —CF₂H, —CFH₂, a hydroxyl group, a cyano group, a nitro group, an amidino group, a hydrazine group, a hydrazone group, a carboxylic acid group or a salt thereof, a sulfonic acid group or a salt thereof, a phosphoric acid group or a salt thereof, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₇-C₆₀ alkyl aryl group, a C₇-C₆₀ aryl alkyl group, a C₆-C₆₀ aryloxy group, a C₆-C₆₀ arylthio group, a C₁-C₆₀ heteroaryl group, a C₂-C₆₀ alkyl heteroaryl group, a C₂-C₆₀ heteroaryl alkyl group, a C₁-C₆₀ heteroaryloxy group, a C₁-C₆₀ heteroarylthio group, a monovalent non-aromatic condensed polycyclic group, a monovalent non-aromatic condensed heteropolycyclic group, —N(Q₁₁)(Q₁₂), —Si(Q₁₃)(Q₁₄)(Q₁₅), —B(Q₁₆)(Q₁₇), —C(═O)(Q₁₆), —S(═O)₂(Q₁₆), —P(═O)(Q₁₈)(Q₁₉), —P(Q₁₈)(Q₁₉), or a combination thereof;

a C₃-C₁₀ cycloalkyl group, a C₁₁-C₁₀ heterocycloalkyl group, a C₃-C₁₀ cycloalkenyl group, a C₁-C₁₀ heterocycloalkenyl group, a C₆-C₆₀ aryl group, a C₇-C₆₀ alkyl aryl group, a C₆-C₆₀ aryloxy group, a C₆-C₆₀ arylthio group, a C₁-C₆₀ heteroaryl group, a C₂-C₆₀ alkyl heteroaryl group, a C₁-C₆₀ heteroaryloxy group, a C₁-C₆₀ heteroarylthio group, a monovalent non-aromatic condensed polycyclic group, or a monovalent non-aromatic condensed heteropolycyclic group;

a C₃-C₁₀ cycloalkyl group, a C₁₁-C₁₀ heterocycloalkyl group, a C₃-C₁₀ cycloalkenyl group, a C₁-C₁₀ heterocycloalkenyl group, a C₆-C₆₀ aryl group, a C₇-C₆₀ alkyl aryl group, a C₆-C₆₀ aryloxy group, a C₆-C₆₀ arylthio group, a C₁-C₆₀ heteroaryl group, a C₂-C₆₀ alkyl heteroaryl group, a C₁-C₆₀ heteroaryloxy group, a C₁-C₆₀ heteroarylthio group, a monovalent non-aromatic condensed polycyclic group, and a monovalent non-aromatic condensed heteropolycyclic group, each substituted with deuterium, —F, —Cl, —Br, —I, —CD₃, —CD₂H, —CDH₂, —CF₃, —CF₂H, —CFH₂, a hydroxyl group, a cyano group, a nitro group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkoxy group, a C₁-C₆₀ alkylthio group, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₇-C₆₀ alkyl aryl group, a C₇-C₆₀ aryl alkyl group, a C₆-C₆₀ aryloxy group, a C₆-C₆₀ arylthio group, a C₁-C₆₀ heteroaryl group, a C₂-C₆₀ alkyl heteroaryl group, a C₂-C₆₀ heteroaryl alkyl group, a C₁-C₆₀ heteroaryloxy group, a C₁-C₆₀ heteroarylthio group, a monovalent non-aromatic condensed polycyclic group, a monovalent non-aromatic condensed heteropolycyclic group, —N(Q₂₁)(Q₂₂), —Si(Q₂₃)(Q₂₄)(Q₂₅), —B(Q₂₆)(Q₂₇), —C(═O)(Q₂₆), —S(═O)₂(Q₂₆), —P(═O)(Q₂₈)(Q₂₉), —P(Q₂₈)(Q₂₉), or a combination thereof; or

—N(Q₃₁)(Q₃₂), —Si(Q₃₃)(Q₃₄)(Q₃₅), —B(Q₃₆)(Q₃₇), —C(═O)(Q₃₆), —S(═O)₂(Q₃₆), —P(═O)(Q₃₈)(Q₃₉), or —P(Q₃₈)(Q₃₉),

wherein Q₁ to Q₉, Q₁₁ to Q₁₉, Q₂₁ to Q₂₉, and Q₃₁ to Q₃₉ may each independently be: hydrogen, deuterium, —F, —Cl, —Br, —I, a hydroxyl group, a cyano group, a nitro group, an amidino group, a hydrazine group, a hydrazone group, a carboxylic acid group or a salt thereof, a sulfonic acid group or a salt thereof, a phosphoric acid group or a salt thereof; a C₁-C₆₀ alkyl group substituted with a C₁-C₆₀ alkyl group, deuterium, a C₁-C₆₀ alkyl group, a C₆-C₆₀ aryl group, or a combination thereof; a C₂-C₆₀ alkenyl group; a C₂-C₆₀ alkynyl group; a C₁-C₆₀ alkoxy group; a C₃-C₁₀ cycloalkyl group; a C₁-C₁₀ heterocycloalkyl group; a C₃-C₁₀ cycloalkenyl group; a C₂-C₁₀ heterocycloalkenyl group; a C₆-C₆₀ aryl group; a C₆-C₆₀ aryl group substituted with deuterium, a C₁-C₆₀ alkyl group, a C₆-C₆₀ aryl group, or a combination thereof; a C₆-C₆₀ aryloxy group; a C₆-C₆₀ arylthio group; a C₁-C₆₀ heteroaryl group; a C₂-C₆₀ alkyl heteroaryl group; a C₂-C₆₀ heteroaryl alkyl group; a C₁-C₆₀ heteroaryloxy group; a C₁-C₆₀ heteroarylthio group; a monovalent non-aromatic condensed polycyclic group; or a monovalent non-aromatic condensed heteropolycyclic group.

The “C₅-C₃₀ carbocyclic group (unsubstituted or substituted with at least one R₁₀ₐ)” and the “C₁-C₃₀ heterocyclic group (unsubstituted or substituted with at least one R₁₀ₐ)” as used herein may be, for example, a cyclopentane group, a cyclohexane group, a cycloheptane group, a cyclopentene group, a cyclohexene group, a cycloheptene group, an adamantane group, a norbornane group, a norbornene group, a bicyclo[1.1.1]pentane group, a bicyclo[2.1.1]hexane group, a bicyclo[2.2.1]heptane group, a bicyclo[2.2.2]octane group, a benzene group, a naphthalene group, an anthracene group, a phenanthrene group, a triphenylene group, a pyrene group, a chrysene group, a 1,2,3,4-tetrahydronaphthalene group, a pyrrole group, a borole group, a phosphole group, a cyclopentadiene group, a silole group, a germole group, a thiophene group, a selenophene group, a furan group, an indole group, a benzoborole group, a benzophosphole group, an indene group, a benzosilole group, a benzogermole group, a benzothiophene group, a benzoselenophene group, a benzofuran group, a carbazole group, a dibenzoborole group, a dibenzophosphole group, a fluorene group, a dibenzosilole group, a dibenzogermole group, a dibenzothiophene group, a dibenzoselenophene group, a dibenzofuran group, a dibenzothiophene 5-oxide group, a 9H-fluorene-9-one group, a dibenzothiophene 5,5-dioxide group, an azaindole group, an azabenzoborole group, an azabenzophosphole group, an azaindene group, an azabenzosilole group, an azabenzogermole group, an azabenzothiophene group, an azabenzoselenophene group, an azabenzofuran group, an azacarbazole group, an azadibenzoborole group, an azadibenzophosphole group, an azafluorene group, an azadibenzosilole group, an azadibenzogermole group, an azadibenzothiophene group, an azadibenzoselenophene group, an azadibenzofuran group, an azadibenzothiophene 5-oxide group, an aza-9H-fluorene-9-one group, an azadibenzothiophene 5,5-dioxide group, a pyridine group, a pyrimidine group, a pyrazine group, a pyridazine group, a triazine group, a quinoline group, an isoquinoline group, a benzoquinoline group, a benzoisoquinoline group, a quinoxaline group, a quinazoline group, a phenanthroline group, a pyrazole group, an imidazole group, a triazole group, an azaborole group, an azaphosphole group, an azacyclopentadiene group, an azasilole group, an azagermole group, an azaselenophene group, an oxazole group, an isooxazole group, a thiazole group, an isothiazole group, an oxadiazole group, a thiadiazole group, a benzopyrazole group, a benzimidazole group, a benzoxazole group, a benzothiazole group, a benzoxadiazole group, a benzothiadiazole group, a 5,6,7,8-tetrahydroisoquinoline group, or a 5,6,7,8-tetrahydroquinoline group, each (unsubstituted or substituted with at least one R₁₀ₐ).

Hereinafter, a compound and an organic light-emitting device according to embodiments are described in detail with reference to Synthesis Example and Examples. However, the organic light-emitting device is not limited thereto. The wording “‘B’ was used instead of ‘A’” used in describing Synthesis Examples means that an amount of ‘A’ used was identical to an amount of ‘B’ used, in terms of a molar equivalent.

### EXAMPLES

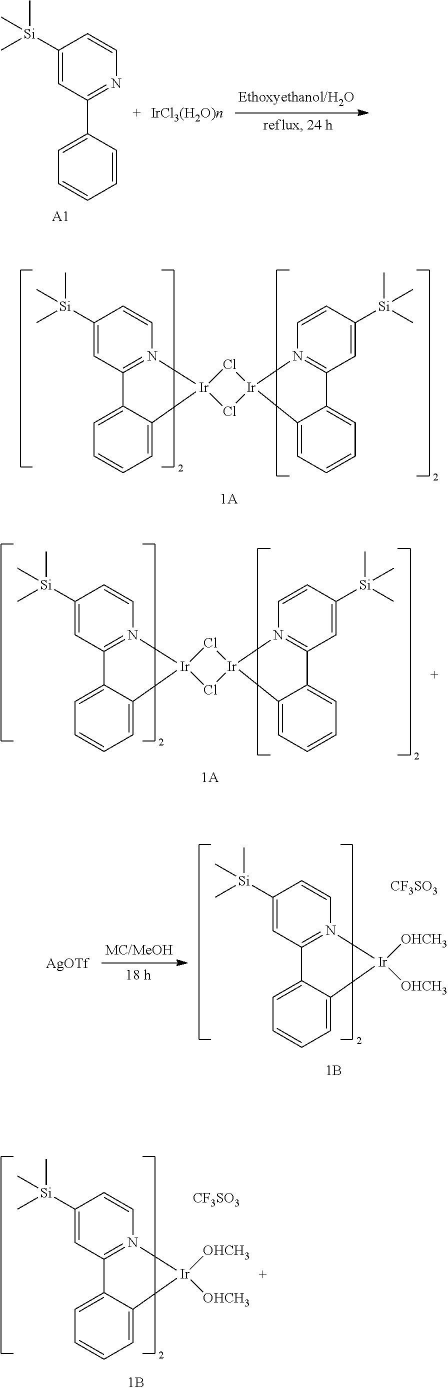

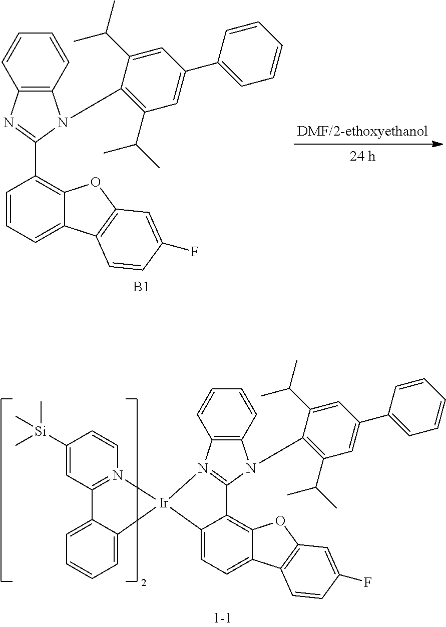

### (1) Synthesis of Compound 1A

2-phenyl-4-(trimethylsilyl)pyridine (4.41 grams (g), 19.40 millimoles (mmol)) and iridium chloride (3.1 g, 8.82 mmol) were mixed with 33 milliliters (mL) of ethoxyethanol and 11 mL of deionized water (DI) and then, stirred under reflux for 24 hours, and then the temperature was lowered to room temperature. The resulting solid was separated by filtration, washed sufficiently with water, methanol, and hexane, in this stated order, and then dried in a vacuum oven to obtain 5.2 g (yield of 87%) of Compound 1A.

### (2) Synthesis of Compound 1B

Compound 1A (2.38 g, 1.75 mmol) and 63 mL of methylene chloride were mixed, and then, silver triflate (AgOTf) (0.898 g, 3.50 mmol) was added thereto after being mixed with 21 mL of methanol. Thereafter, the mixture was stirred for 18 hours at room temperature while light was blocked with aluminum foil, and then filtered through Celite to remove the resulting solid, and reduced pressure was applied to the filtrate to obtain a solid (Compound 1B). Compound 1B was used in the next reaction without an additional purification process.

### (3) Synthesis of Compound 1-1

Compound 1B (2.46 g, 2.87 mmol) and 1-(3,5-diisopropyl-[1,1′-biphenyl]-4-yl)-2-(7-fluorodibenzo[b,d]furan-4-yl)-1H-benzo[d]imidazole (1.549 g, 2.87 mmol) were mixed with 25 mL of 2-ethoxyethanol and 25 mL of dimethylformamide, stirred under reflux for 24 hours at a temperature of 130° C., and then the temperature was reduced to room temperature. Reduced pressure was then applied to the mixture to obtain a solid, which was then purified by column chromatography using a solvent mixture of ethyl acetate (EA) and hexanes (EA:hexanes mixture) to thereby obtain 1.46 g of Compound 1-1 (yield of 43%). The obtained compound was identified by high resolution mass spectrometry (HRMS, using matrix assisted laser desorption ionization (MALDI)) and high-performance liquid chromatography (HPLC) analysis.

HRMS (MALDI) calcd for C₆₅H₆₂FIrN₄OSi₂: m/z 1182.4075 Found: 1182.4074.

### Synthesis Example 2: Synthesis of Compound 23-14

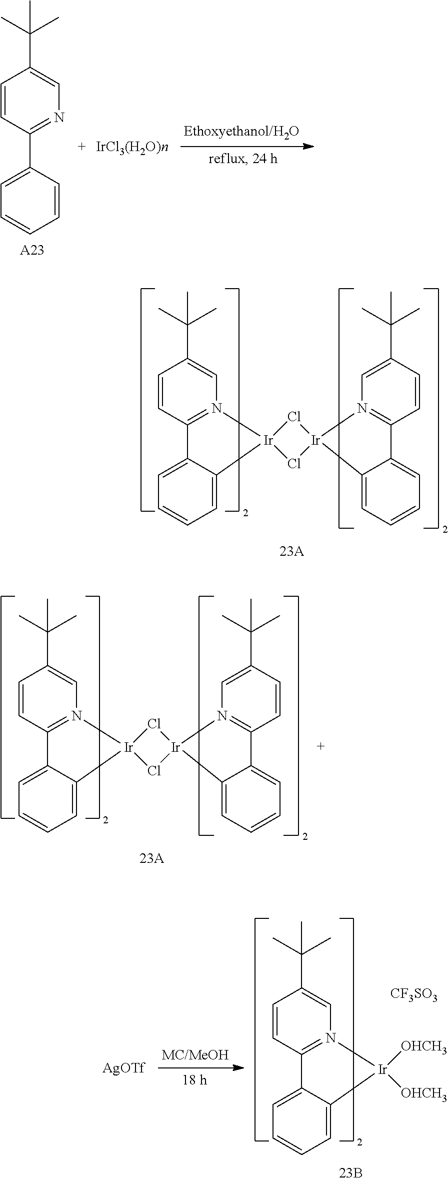

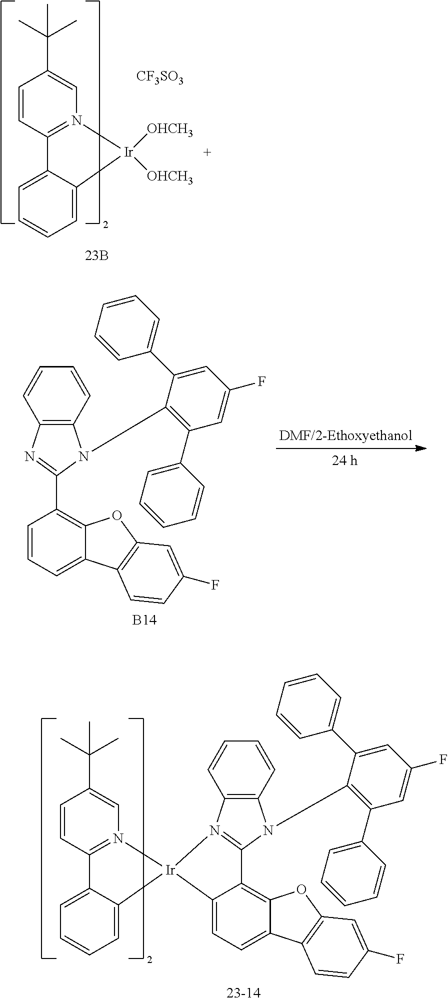

### (1) Synthesis of Compound 23A

3.8 g of Compound 23A (yield of 76%) was obtained by using a similar method to that used in synthesizing Compound 1A of Synthesis Example 1, except that 5-(tert-butyl)-2-phenylpyridine was used instead of 2-phenyl-4-(trimethylsilyl)pyridine.

### (2) Synthesis of Compound 23B

Compound 23B was obtained by using a similar method to that used in synthesizing Compound 1B of Synthesis Example 1, except that Compound 23A was used instead of Compound 1A. The obtained Compound 23B was used in the next reaction without any additional purification process.

### (3) Synthesis of Compound 23-14

Compound 23B (2.85 g, 3.45 mmol) and 1-(5′-fluoro-[1,1′:3′,1″-terphenyl]-2′-yl)-2-(7-fluorodibenzo[b,d]furan-4-yl)-1H-benzo[d]imidazole (1.89 g, 3.45 mmol) were mixed with 35 mL of 2-ethoxyethanol and 35 mL of dimethylformamide, and then the resulting mixture was stirred under reflux for 24 hours at a temperature of 130° C. The temperature was then reduced to room temperature. Reduced pressure was applied to the obtained mixture to obtain a solid, which was then purified by column chromatography in an EA:hexanes mixture to thereby obtain 1.88 g of Compound 23-14 (yield of 47%). The obtained compound was identified by HRMS and HPLC analysis.

HRMS (MALDI) calcd for C₆₇H₅₃F₂IrN₄O: m/z 1160.3817 Found: 1160.3815.

### Synthesis Example 3: Synthesis of Compound 149-15

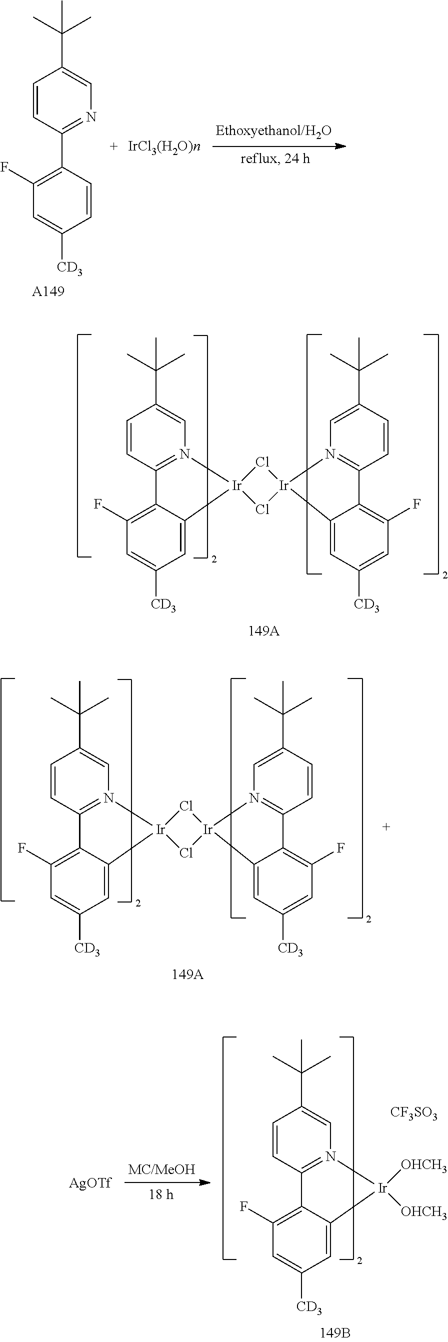

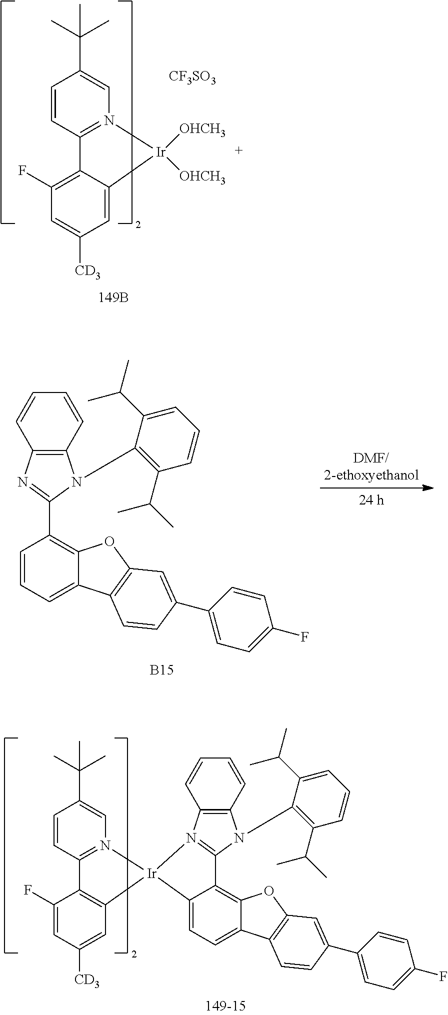

### (1) Synthesis of Compound 149A

4.30 g of Compound 149A (yield of 86%) was obtained by using a similar method to that used in synthesizing Compound 1A of Synthesis Example 1, except that 5-(tert-butyl)-2-(2-fluoro-4-(methyl-d3)phenyl)pyridine was used instead of 2-phenyl-4-(trimethylsilyl)pyridine.

### (2) Synthesis of Compound 149B

Compound 149B was obtained by using a similar method to that used in synthesizing Compound 1B of Synthesis Example 1, except that Compound 149A was used instead of Compound 1A. The obtained Compound 149B was used in the next reaction without an additional purification process.

### (3) Synthesis of Compound 149-14

Compound 149B (3.30 g, 3.69 mmol) and 1-(2,6-diisopropylphenyl)-2-(7-(4-fluorophenyl)dibenzo[b,d]furan-4-yl)-1H-benzo[d]imidazole (1.97 g, 3.69 mmol) were mixed with 37 mL of 2-ethoxyethanol and 37 mL of dimethylformamide to form a mixture, and the mixture was then stirred under reflux for 24 hours at a temperature of 130° C. The temperature was then reduced to room temperature. Reduced pressure was applied to the mixture to obtain a solid, which was then purified by column chromatography in an EA:hexanes mixture to thereby obtain 1.91 g of Compound 149-14 (yield of 42%). The obtained compound was identified by HRMS and HPLC analysis.

HRMS (MALDI) calcd for Cs₉H₅₈D₆F₃IrN₄O: m/z 1220.5038 Found: 1220.5039.

### Synthesis Example 4: Synthesis of Compound 135-295

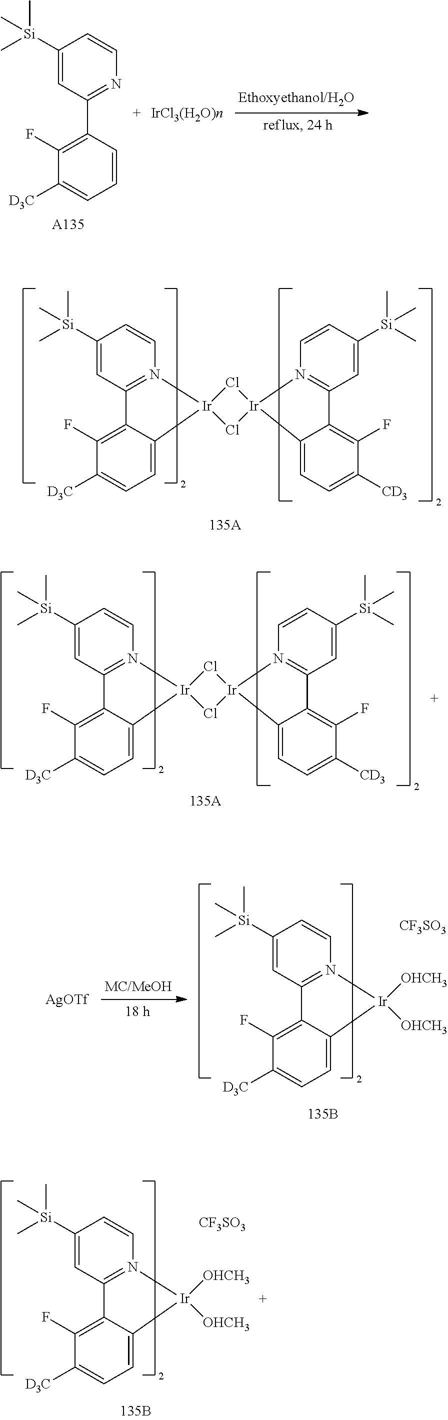

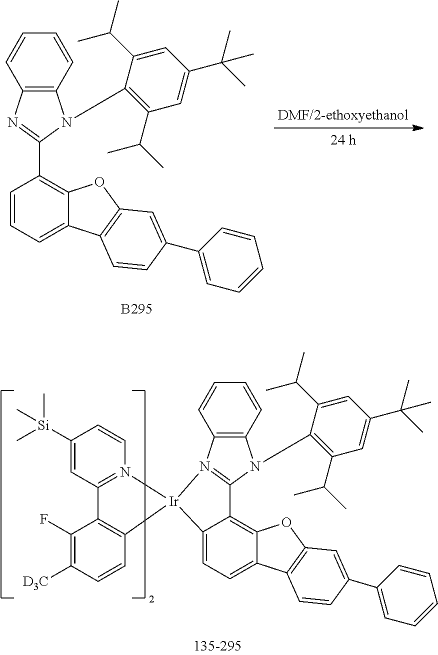

### (1) Synthesis of Compound 135A

3.57 g of Compound 135A (yield of 89%) was obtained by using a similar method that that used in synthesizing Compound 1A of Synthesis Example 1, except that 2-(2-fluoro-3-(methyl-d3)phenyl)-4-(trimethylsilyl)pyridine was used instead of 2-phenyl-4-(trimethylsilyl)pyridine.

### (2) Synthesis of Compound 135B

Compound 135B was obtained by using a similar method to that used in synthesizing Compound 1B of Synthesis Example 1, except that Compound 135A was used instead of Compound 1A. The obtained Compound 135B was used in the next reaction without an additional purification process.

### (3) Synthesis of Compound 135-295

Compound 135B (2.88 g, 3.10 mmol) and 1-(4-(tert-butyl)-2,6-diisopropylphenyl)-2-(7-phenyldibenzo[b,d]furan-4-yl)-1H-benzo[d]imidazole (1.79 g, 3.10 mmol) were mixed with 30 mL of 2-ethoxyethanol and 30 mL of dimethylformamide to form a mixture, and then the mixture was stirred under reflux for 24 hours at a temperature of 130° C. The temperature was then reduced to room temperature. Reduced pressure was applied to the obtained mixture to obtain a solid, which was then purified by column chromatography in an EA:hexanes mixture to thereby obtain 1.76 g of Compound 135-295 (yield of 44%). The obtained compound was identified by HRMS and HPLC analysis.

HRMS (MALDI) calcd for C₇₁H₆₇D₆F₂IrN₄OSi₂: m/z 1290.5297 Found: 1290.5297.

### Synthesis Example 5: Synthesis of Compound 148-53

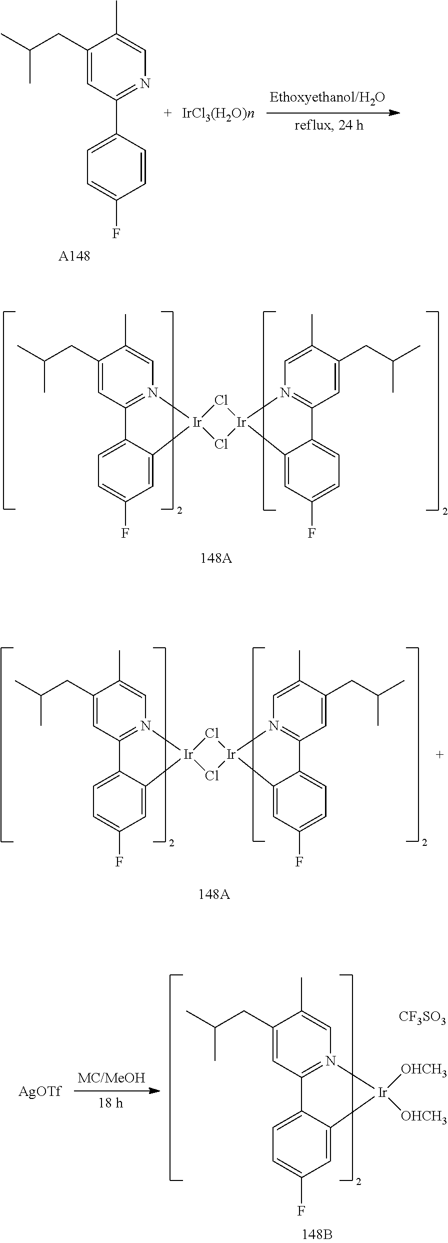

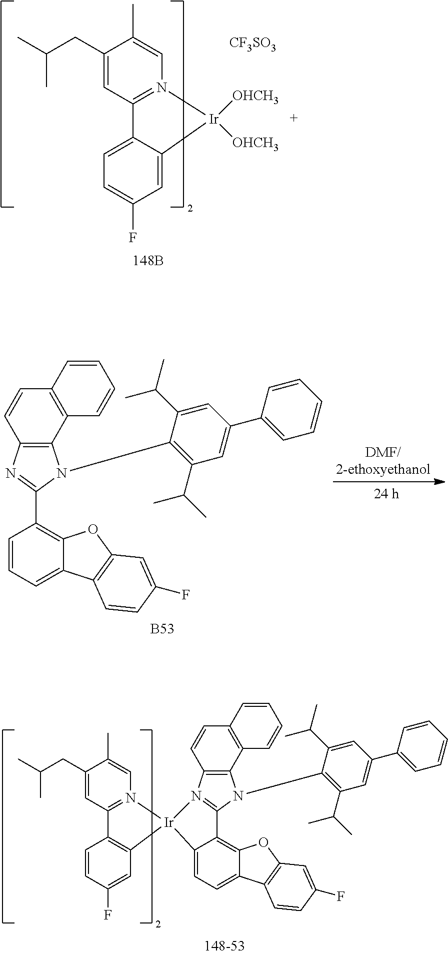

### (1) Synthesis of Compound 148B G-17C

3.0 g of Compound 148A (yield of 80%) was obtained by using a similar method to that used in synthesizing Compound 1B of Synthesis Example 1, except that 4-isobutyl-5-methyl-2-phenylpyridine was used instead of 2-phenyl-4-(trimethylsilyl)pyridine.

### (2) Synthesis of Compound 148-5

Compound 148B (2.92 g, 3.42 mmol) and 1-(3,5-diisopropyl-[1,1′-biphenyl]-4-yl)-2-(7-fluorodibenzo[b,d]furan-4-yl)-1H-naphtho[1,2-d]imidazole (2.01 g, 3.42 mmol) were mixed with 34 mL of 2-ethoxyethanol and 34 mL of dimethylformamide to form a mixture, and then the mixture stirred under reflux for 24 hours at a temperature of 130° C. The temperature was then reduced to room temperature. Reduced pressure was applied to the obtained mixture to obtain a solid, which was then purified by column chromatography in an EA:hexanes mixture to thereby obtain 1.69 g of Compound 148-53 (yield of 40%). The obtained compound was identified by HRMS and HPLC analysis.

HRMS (MALDI) calcd for C₇₃H₆₆F₃IrN₄O: m/z 1264.4818 Found: 1264.4815.

### Synthesis Example 6: Synthesis of Compound 218-1

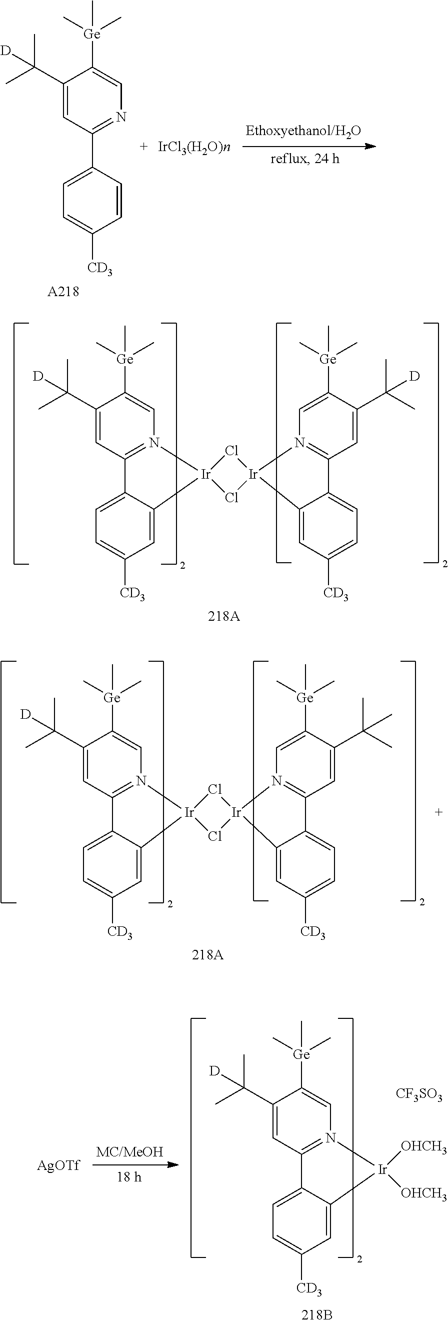

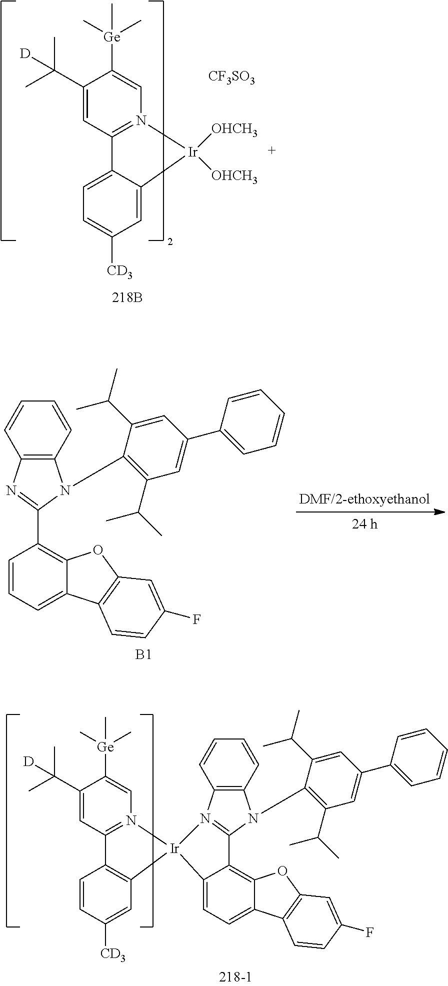

### (1) Synthesis of Compound 218A

3.64 g of Compound 218A (yield of 81%) was obtained by using a similar method to that used in synthesizing Compound 1A of Synthesis Example 1, except that 2-(4-(methyl-d3)phenyl)-4-(propan-2-yl-2-d)-5-(trimethylgermyl)pyridine was used instead of 2-phenyl-4-(trimethylsilyl)pyridine.

### (2) Synthesis of Compound 218B

Compound 218B was obtained by using a similar method to that used in synthesizing Compound 1B of Synthesis Example 1, except that Compound 218A was used instead of Compound 1A. The obtained Compound 218B was used in the next reaction without an additional purification process.

### (3) Synthesis of Compound 218-1

Compound 218B (2.68 g, 2.51 mmol) and 1-(3,5-diisopropyl-[1,1′-biphenyl]-4-yl)-2-(7-fluorodibenzo[b,d]furan-4-yl)-1H-benzo[d]imidazole (1.35 g, 2.51 mmol) were mixed with 25 mL of 2-ethoxyethanol and 25 mL of dimethylformamide to form a mixture, and the mixture was then stirred under reflux for 24 hours at a temperature of 130° C. The temperature was then reduced to room temperature. Reduced pressure was applied to the obtained mixture to obtain a solid, which was then purified by column chromatography in an EA:hexanes mixture to thereby obtain 1.45 g of Compound 218-1 (yield of 41%). The obtained compound was identified by HRMS and HPLC analysis.

HRMS (MALDI) calcd for C₇₃H₇₀D₈FGe₂IrN₄O: m/z 1394.4715 Found: 1394.4714.

### Synthesis Example 7: Synthesis of Compound 245-411

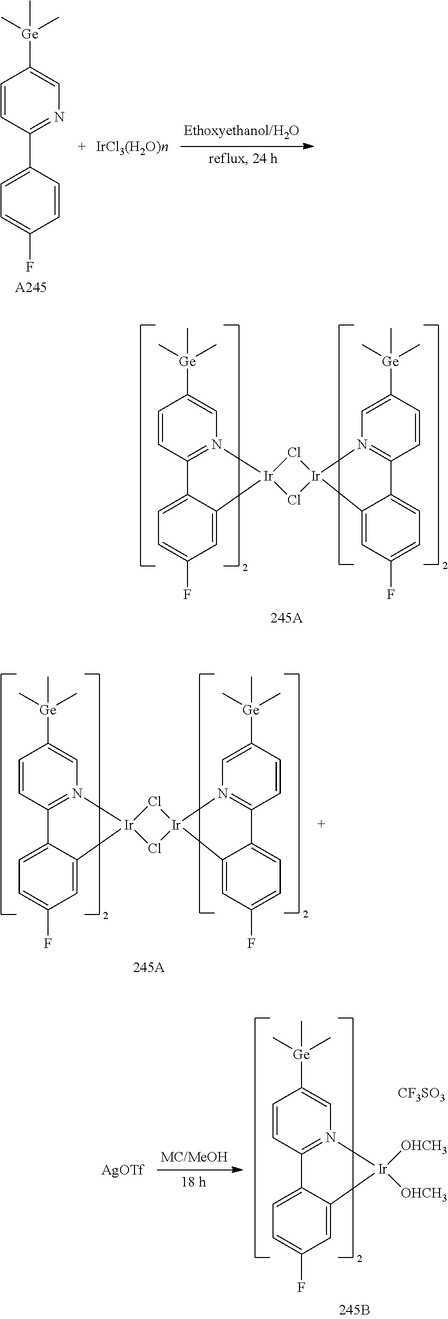

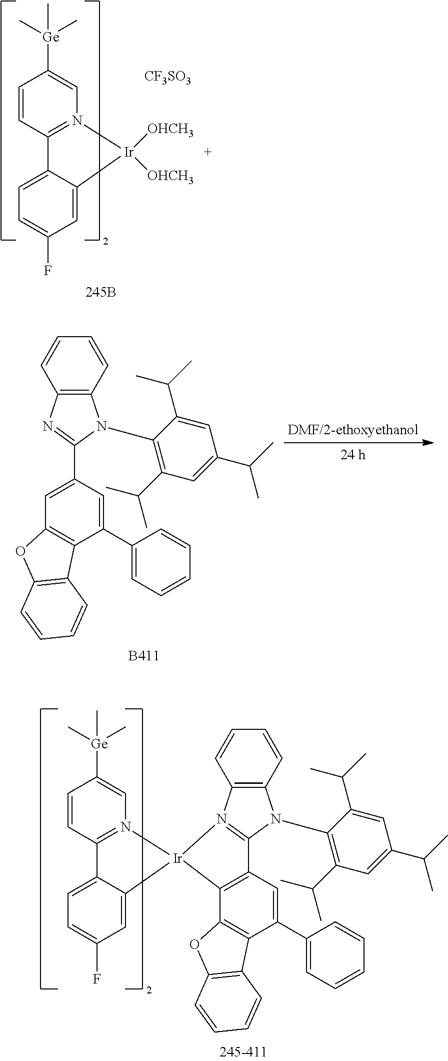

### (1) Synthesis of Compound 245A

3.85 g of Compound 245A (yield of 86%) was obtained by using a similar method to that used in synthesizing Compound 1A of Synthesis Example 1, except that 2-(4-fluorophenyl)-5-(trimethylgermyl)pyridine was used instead of 2-phenyl-4-(trimethylsilyl)pyridine.

### (2) Synthesis of Compound 245B

Compound 245B was obtained by using a similar method to that used in synthesizing Compound 1B of Synthesis Example 1, except that Compound 245A was used instead of Compound 1A. Obtained Compound 245B was used in the next reaction without an additional purification process.

### (3) Synthesis of Compound 245-411

Compound 245B (2.19 g, 2.63 mmol) and 2-(1-phenyldibenzo[b,d]furan-3-yl)-1-(2,4,6-triisopropylphenyl)-1H-benzo[d]imidazole)(1.48 g, 2.63 mmol) were mixed with 25 mL of 2-ethoxyethanol and 25 mL of dimethylformamide to form a mixture, and then the mixture was stirred under reflux for 24 hours at a temperature of 130° C. The temperature was then reduced to room temperature. Reduced pressure was applied to the obtained mixture to obtain a solid, which was then purified by column chromatography in an EA:hexanes mixture to thereby obtain 1.20 g of Compound 245-411 (yield of 34%). The obtained compound was identified by HRMS and HPLC analysis.

HRMS (MALDI) calcd for Cs₈H₆₇F₂Ge₂IrN₄O: m/z 1334.3336 Found: 1334.3337.

### Example 1

As an anode, an ITO-patterned glass substrate was cut to a size of 50 millimeters (mm)×50 mm×0.5 mm, sonicated with isopropyl alcohol and DI water, each for 5 minutes, and then cleaned by exposure to ultraviolet (UV) rays and ozone for 30 minutes. The resultant glass substrate was loaded onto a vacuum deposition apparatus.

Compounds HT3 and F6-TCNNQ were co deposited by vacuum on the anode to a weight ratio of 98:2 to form a hole injection layer having a thickness of 100 Å, and Compound HT3 was vacuum-deposited on the hole injection layer to form a hole transport layer having a thickness of 1,350 Å, and then, Compound H-H1 was vacuum-deposited on the hole transport layer to form an electron blocking layer having a thickness of 300 Å.

Subsequently, Compounds H-H1, H-E43, and 1-1(dopant) were co-deposited to a weight ratio of 57:38:5 on the electron blocking layer to form an emission layer having a thickness of 400 Å.

Then, Compounds ET3 and ET-D1 were co-deposited to a volume ratio of 50:50 on the emission layer to form an electron transport layer having a thickness of 350 Å, and ET-D1 was vacuum-deposited on the electron transport layer to form an electron injection layer having a thickness of 10 Å, and Al was vacuum-deposited on the electron injection layer to form a cathode having a thickness of 1,000 Å, thereby completing the manufacture of an organic light-emitting device.

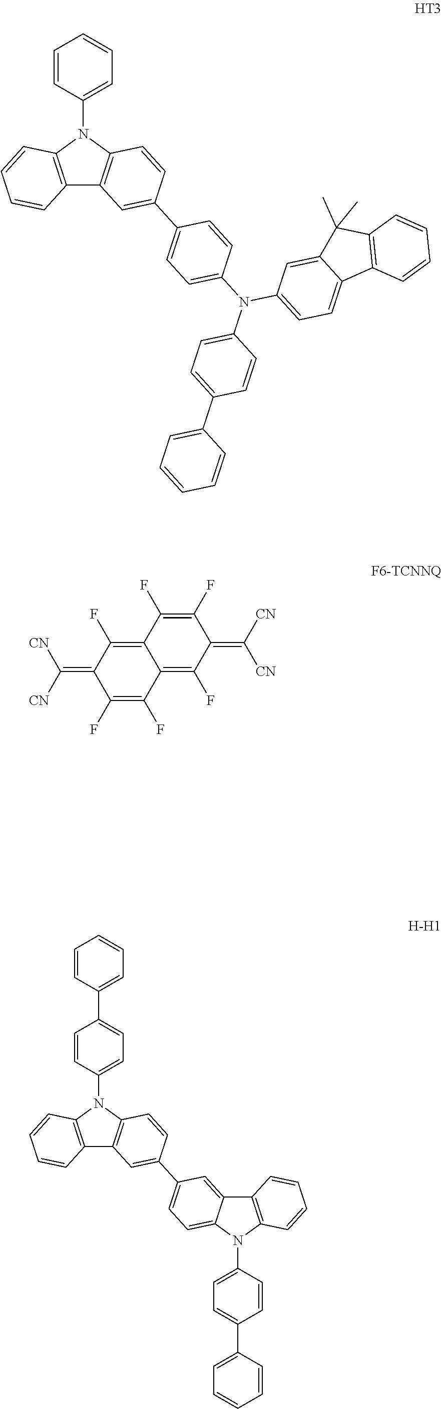

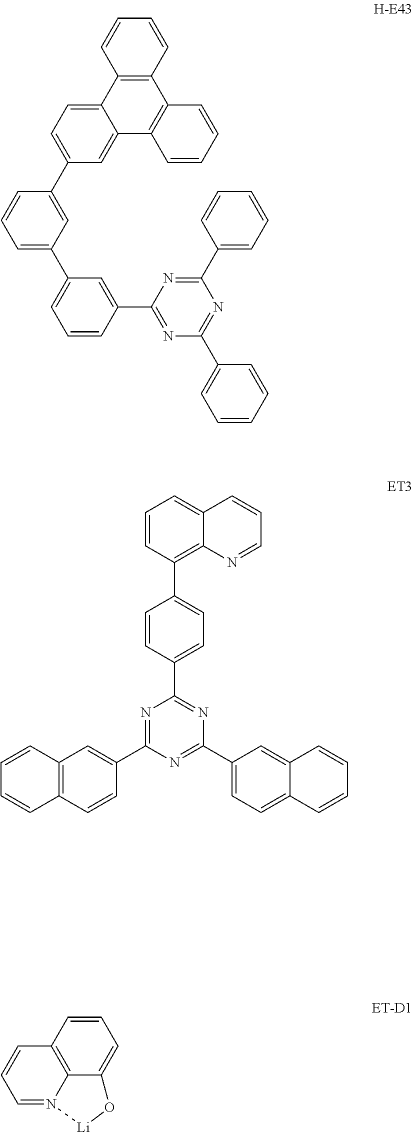

### Examples 2 to 7, Comparative Examples 1 and 2

Organic light-emitting devices were manufactured in the same manner as in Example 1, except that the compounds shown in Table 58 were each used instead of Compound 1 as a dopant when the emission layer was formed.

The driving voltage (volts, V), luminous efficiency (candela per ampere (cd/A), a relative value (%)), and power efficiency (candela per watt (cd/W), a relative value (%)) as measured at 8,000 candela per square meter (cd/m², or nit) of each of the organic light-emitting devices of Examples 1 to 7 and Comparative Examples 1 and 2 were evaluated, and the results thereof are shown in Table 2. As evaluation apparatuses, a current-voltage meter (Keithley 2400) and a luminance meter (Minolta Cs-1000 Å) were used. The roll-off ratio (%) was calculated according to Equation 20 below.

Roll-off ratio={1−(efficiency(at 18,000 cd/m²)/maximum emission efficiency)}×100% Equation 20

TABLE 58

| | | | | Luminous | Power |
|---|---|---|---|---|---|
| | Emission | Driving | Roll-off | efficiency | efficiency |
| | layer dopant | voltage | ratio | (cd/A, | (cd/W, |
| | compound | (V) | (%) | relative value) | relative value) |
| Example 1 | 1-1 | 3.8 | 10% | 97% | 91% |
| Example 2 | 23-14 | 4.1 | 12% | 92% | 81% |
| Example 3 | 149-15 | 3.8 | 6% | 100% | 94% |
| Example 4 | 135-295 | 3.6 | 7% | 100% | 100% |
| Example 5 | 148-53 | 3.5 | 9% | 88% | 91% |
| Example 6 | 218-1 | 4.1 | 10% | 97% | 85% |
| Example 7 | 245-411 | 3.8 | 8% | 88% | 84% |
| Comparative | A | 4.4 | 14% | 86% | 70% |
| Example 1 | | | | | |
| Comparative | B | 4.6 | 15% | 79% | 61% |
| Example 2 | | | | | |

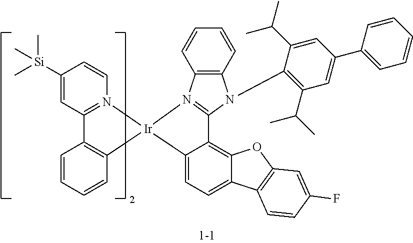

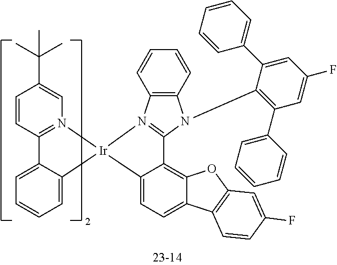

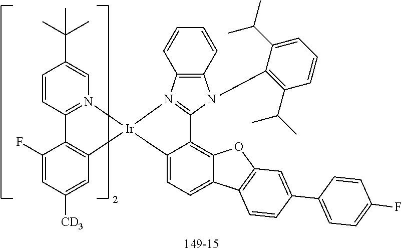

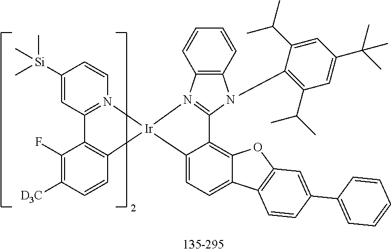

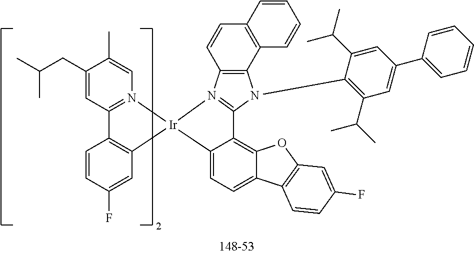

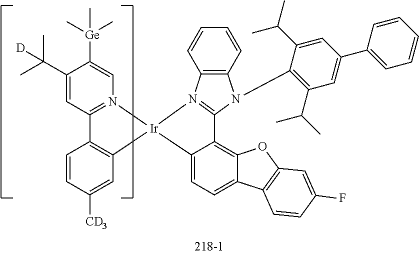

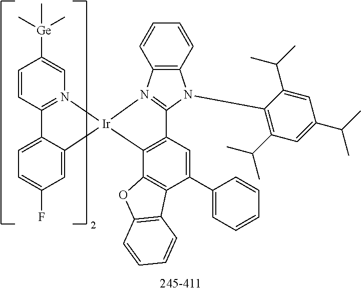

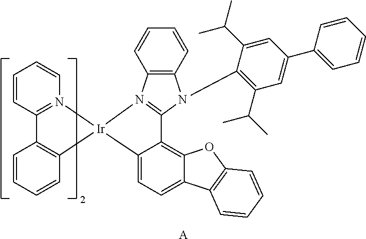

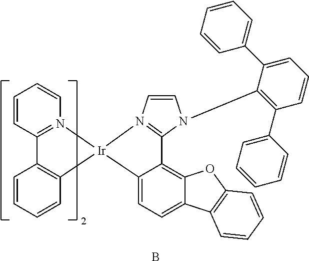

Table 58 shows that the organic light-emitting devices of Examples 1 to 7 have lower driving voltage and excellent current efficiency and power efficiency. In addition, the organic light-emitting devices of Examples 1 to 7 showed lower driving voltage, better luminous efficiency, and better power efficiency than those of the organic light-emitting devices of Comparative Examples 1 and 2.

The organometallic compounds have excellent electrical characteristics and thermal stability. The organometallic compounds have a high glass transition temperature so that crystallization thereof can be reduced or prevented and electric mobility thereof can be improved. Accordingly, an electronic device using the organometallic compounds, for example, an organic light-emitting device using the organometallic compounds, has a low driving voltage, high efficiency, a long lifespan, reduced roll-off ratio, and a relatively narrow EL spectrum emission peak FWHM.

Thus, due to the use of the organometallic compounds, a high-quality organic light-emitting device may be embodied. Such organometallic compounds have excellent phosphorescence-emitting characteristics, and thus, when used, a diagnostic composition having a high diagnostic efficiency may be provided.

It should be understood that embodiments described herein should be considered in a descriptive sense only and not for purposes of limitation. Descriptions of features or aspects within each exemplary embodiment should typically be considered as available for other similar features or aspects in other exemplary embodiments. While one or more exemplary embodiments have been described with reference to the drawing, it will be understood by those of ordinary skill in the art that various changes in form and details may be made therein without departing from the spirit and scope as defined by the following claims.

What is claimed is:

1. An organometallic compound represented by Formula 1:
M₁(Ln₁)ₙ₁(Ln₂)ₙ₂ Formula 1
wherein, in Formula 1,
M₁ is a transition metal,
Ln₁ is a ligand represented by Formula 1A,
Ln₂ is a ligand represented by Formula 1B,
n1 is 1 or 2, and
n2 is 1, 2, or 3, 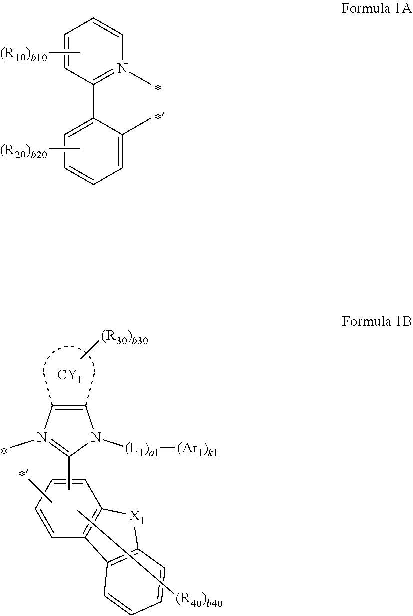
wherein, in Formulae 1A and 1B,
X₁ is O, S, or Se,
CY₁ is a C₅-C₃₀ carbocyclic group or a C₁-C₃₀ heterocyclic group,
L₁ is a substituted or unsubstituted C₃-C₁₀ cycloalkylene group, a substituted or unsubstituted C₁-C₁₀ heterocycloalkylene group, a substituted or unsubstituted C₃-C₁₀ cycloalkenylene group, a substituted or unsubstituted C₂-C₁₀ heterocycloalkenylene group, a substituted or unsubstituted C₆-C₆₀ arylene group, a substituted or unsubstituted C₁-C₆₀ heteroarylene group, a substituted or unsubstituted divalent non-aromatic condensed polycyclic group, or a substituted or unsubstituted divalent non-aromatic condensed heteropolycyclic group,
a1 is 0, 1, 2 or 3,
Ar₁, R₁₀, R₂₀, R₃₀, and R₄₀ are each independently hydrogen, deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₁-C₆₀ alkylthio group, a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₂-C₁₀ heterocycloalkenyl group, a substituted or unsubstituted C₆-C₆₀ aryl group, a substituted or unsubstituted C₇-C₆₀ alkyl aryl group, a substituted or unsubstituted C₇-C₆₀ aryl alkyl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted C₂-C₆₀ alkyl heteroaryl group, a substituted or unsubstituted C₂-C₆₀ heteroaryl alkyl group, a substituted or unsubstituted C₁-C₆₀ heteroaryloxy group, a substituted or unsubstituted C₁-C₆₀ heteroarylthio group, a substituted or unsubstituted monovalent non-aromatic condensed polycyclic group, a substituted or unsubstituted monovalent non-aromatic condensed heteropolycyclic group, —N(Q₁)(Q₂), —Si(Q₃)(Q₄)(Q₅), —Ge(Q₃)(Q₄)(Q₅), —B(Q₆)(Q₇), —C(═O)(Q₆), —S(═O)₂(Q₆), —P(═O)(Q₈)(Q₉), or —P(Q₈)(Q₉),
k1 is 1, 2, 3, 4, 5, 6, 7, or 8,
b10 and b20 are each independently 1, 2, 3, or 4,
b30 is 1, 2, 3, 4, 5, 6, 7, or 8,
b40 is 1, 2, 3, 4, 5, or 6,
wherein the organometallic compound comprises at least one —F,
and *′ each indicate a binding site to a neighboring atom,
at least one substituent of the substituted C₁-C₆₀ alkyl group, the substituted C₂-C₆₀ alkenyl group, the substituted C₂-C₆₀ alkynyl group, the substituted C₁-C₆₀ alkoxy group, the substituted C₁-C₆₀ alkylthio group, the substituted C₃-C₁₀ cycloalkyl group, the substituted C₁-C₁₀ heterocycloalkyl group, the substituted C₃-C₁₀ cycloalkenyl group, the substituted C₁-C₁₀ heterocycloalkenyl group, the substituted C₆-C₆₀ aryl group, the substituted C₇-C₆₀ alkyl aryl group, the substituted C₇-C₆₀ aryl alkyl group, the substituted C₆-C₆₀ aryloxy group, the substituted C₆-C₆₀ arylthio group, the substituted C₁-C₆₀ heteroaryl group, the substituted C₂-C₆₀ alkyl heteroaryl group, the substituted C₂-C₆₀ heteroaryl alkyl group, the substituted C₁-C₆₀ heteroaryloxy group, the substituted C₁-C₆₀ heteroarylthio group, the substituted monovalent non-aromatic condensed polycyclic group, and the substituted monovalent non-aromatic condensed heteropolycyclic group is:
deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, or a C₁-C₆₀ alkoxy group;
a C₁-C₆₀ alkyl group, a C₂-C₆₀ alkenyl group, a C₂-C₆₀ alkynyl group, or a C₁-C₆₀ alkoxy group, each substituted with deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₃-C₁₀ cycloalkyl group, a C₁-C₁₀ heterocycloalkyl group, a C₃-C₁₀ cycloalkenyl group, a C₂-C₁₀ heterocycloalkenyl group, a C₆-C₆₀ aryl group, a C₇-C₆₀ alky aryl group, a C₇-C₆₀ aryl alkyl group, a C₆-C₆₀ aryloxy group, a C₆-C₆₀ arylthio group, a C₁-C₆₀ heteroaryl group, a C₂-C₆₀ alkyl heteroaryl group, a C₂-C₆₀ heteroaryl alkyl group, a C₁-C₆₀ heteroaryloxy group, a C₁-C₆₀ heteroarylthio group, a monovalent non-aromatic condensed polycyclic group, a monovalent non-aromatic condensed heteropolycyclic group, —N(Q₁₁)(Q₁₂), —Si(Q₁₃)(Q₁₄)(Q₁₅), —Ge(Q₁₃)(Q₁₄)(Q₁₅), —B(Q₁₆)(Q₁₇), —C(═O)(Q₁₆), —S(═O)₂(Q₁₆), —P(═O)(Q₁₈)(Q₁₉), —P(Q₁₈)(Q₁₉), or a combination thereof;
a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₇-C₆₀ alky aryl group, a C₆-C₆₀ aryloxy group, a C₆-C₆₀ arylthio group, a C₁-C₆₀ heteroaryl group, a C₂-C₆₀ alkyl heteroaryl group, a C₁-C₆₀ heteroaryloxy group, a C₁-C₆₀ heteroarylthio group, a monovalent non-aromatic condensed polycyclic group, or a monovalent non-aromatic condensed heteropolycyclic group;
a C₃-C₁₀ cycloalkyl group, a C₁₁-C₁₀ heterocycloalkyl group, a C₃-C₁₀ cycloalkenyl group, a C₁-C₁₀ heterocycloalkenyl group, a C₆-C₆₀ aryl group, a C₇-C₆₀ alky aryl group, a C₆-C₆₀ aryloxy group, a C₆-C₆₀ arylthio group, a C₁-C₆₀ heteroaryl group, a C₂-C₆₀ alkyl heteroaryl group, a C₁-C₆₀ heteroaryloxy group, a C₁-C₆₀ heteroarylthio group, a monovalent non-aromatic condensed polycyclic group, or a monovalent non-aromatic condensed heteropolycyclic group, each substituted with deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkoxy group, a C₁-C₆₀ alkylthio group, a C₃-C₁₀ cycloalkyl group, a C₁-C₁₀ heterocycloalkyl group, a C₃-C₁₀ cycloalkenyl group, a C₂-C₁₀ heterocycloalkenyl group, a C₆-C₆₀ aryl group, a C₇-C₆₀ alky aryl group, a C₇-C₆₀ aryl alkyl group, a C₆-C₆₀ aryloxy group, a C₆-C₆₀ arylthio group, a C₁-C₆₀ heteroaryl group, a C₂-C₆₀ alkyl heteroaryl group, a C₂-C₆₀ heteroaryl alkyl group, a C₁-C₆₀ heteroaryloxy group, a C₁-C₆₀ heteroarylthio group, a monovalent non-aromatic condensed polycyclic group, a monovalent non-aromatic condensed heteropolycyclic group, —N(Q₂₁)(Q₂₂), —Si(Q₂₃)(Q₂₄)(Q₂₅), —Ge(Q₂₃)(Q₂₄)(Q₂₅), —B(Q₂₆)(Q₂₇), —C(═O)(Q₂₆), —S(═O)₂(Q₂₆), —P(═O)(Q₂₈)(Q₂₉), —P(═O)(Q₂₈)(Q₂₉), or a combination thereof; or
—N(Q₃₁)(Q₃₂), —Si(Q₃₃)(Q₃₄)(Q₃₅), —Ge(Q₃₃)(Q₃₄)(Q₃₅), —B(Q₃₆)(Q₃₇), —C(═O)(Q₃₆), —S(═O)₂(Q₃₆), —P(═O)(Q₃₈)(Q₃₉), or —P(Q₃₈)(Q₃₉),
wherein Q₁ to Q₉, Q₁₁ to Q₁₉, Q₂₁ to Q₂₉, and Q₃₁ to Q₃₉ are each independently hydrogen, deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₁-C₆₀ alkylthio group, a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₁-C₁₀ heterocycloalkenyl group, a substituted or unsubstituted C₆-C₆₀ aryl group, a substituted or unsubstituted C₇-C₆₀ alkyl aryl group, a substituted or unsubstituted C₇-C₆₀ aryl alkyl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted C₂-C₆₀ alkyl heteroaryl group, a substituted C₂-C₆₀ heteroaryl alkyl group, a C₁-C₆₀ heteroaryloxy group, a C₁-C₆₀ heteroarylthio group, a substituted or unsubstituted monovalent non-aromatic condensed polycyclic group, or a substituted or unsubstituted monovalent non-aromatic condensed heteropolycyclic group.

2. The organometallic compound of claim 1, wherein M₁ is iridium (Ir), platinum (Pt), osmium (Os), titanium (Ti), zirconium (Zr), hafnium (Hf), europium (Eu), terbium (Tb), thulium (Tm), or rhodium (Rh).

3. The organometallic compound of claim 1, wherein M₁ is Ir, and the sum of n1 and n2 is 3.

4. The organometallic compound of claim 1, wherein the organometallic compound comprises 1 to 4 of —F.

5. The organometallic compound of claim 1, wherein the organometallic compound is represented by one of Formulae 11-1 to 11-24: 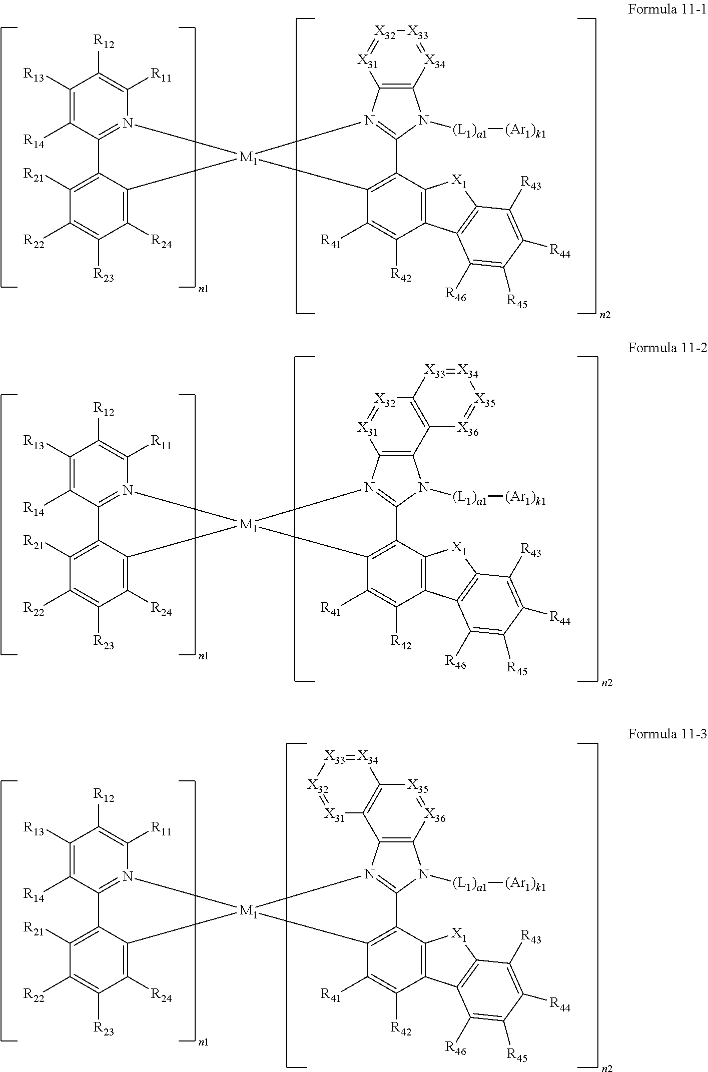 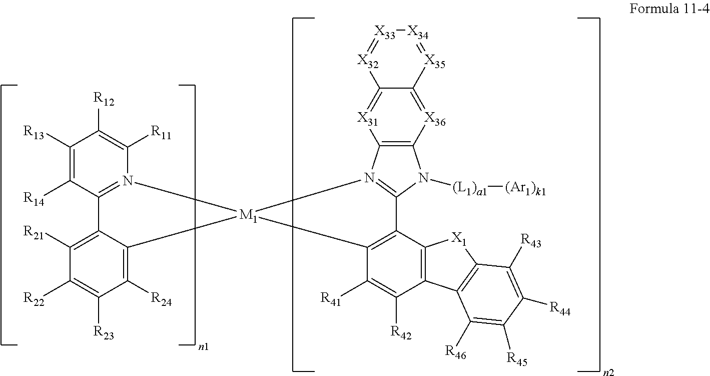 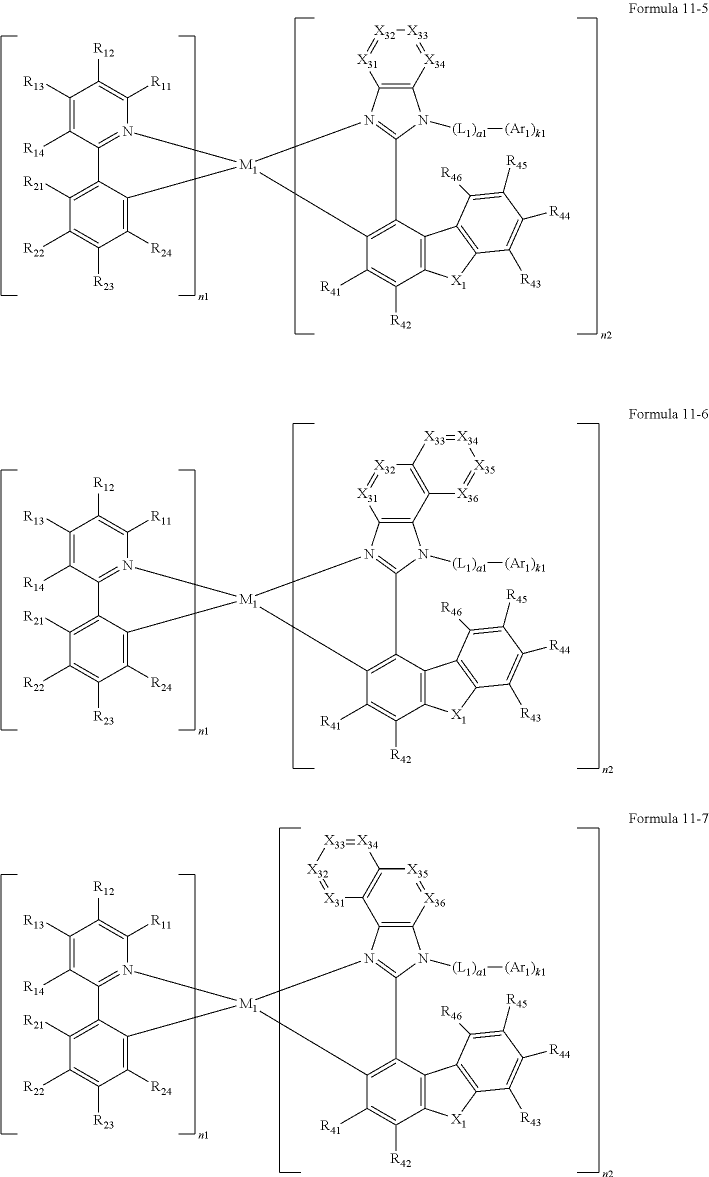 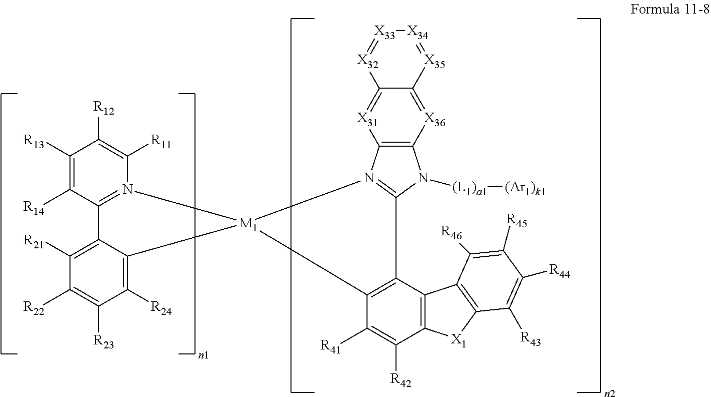 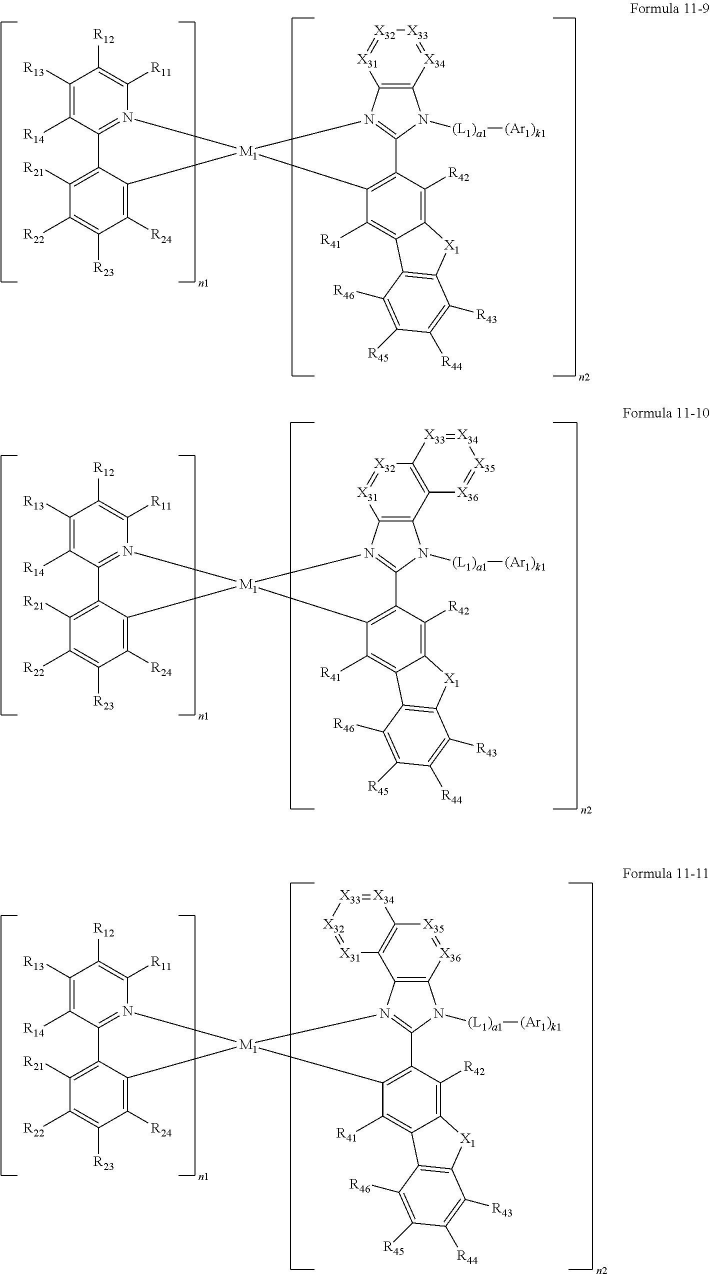 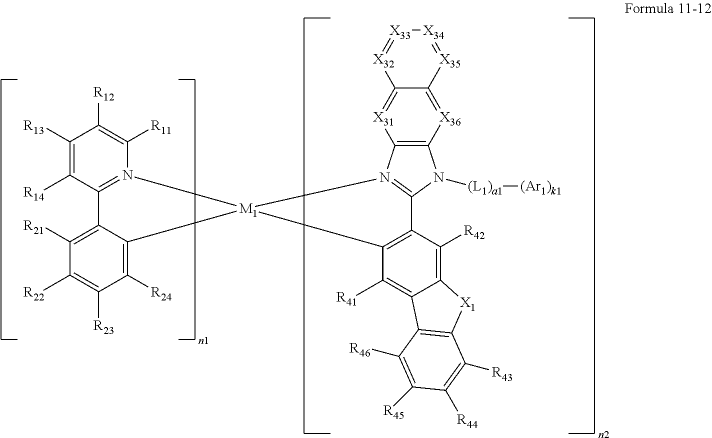 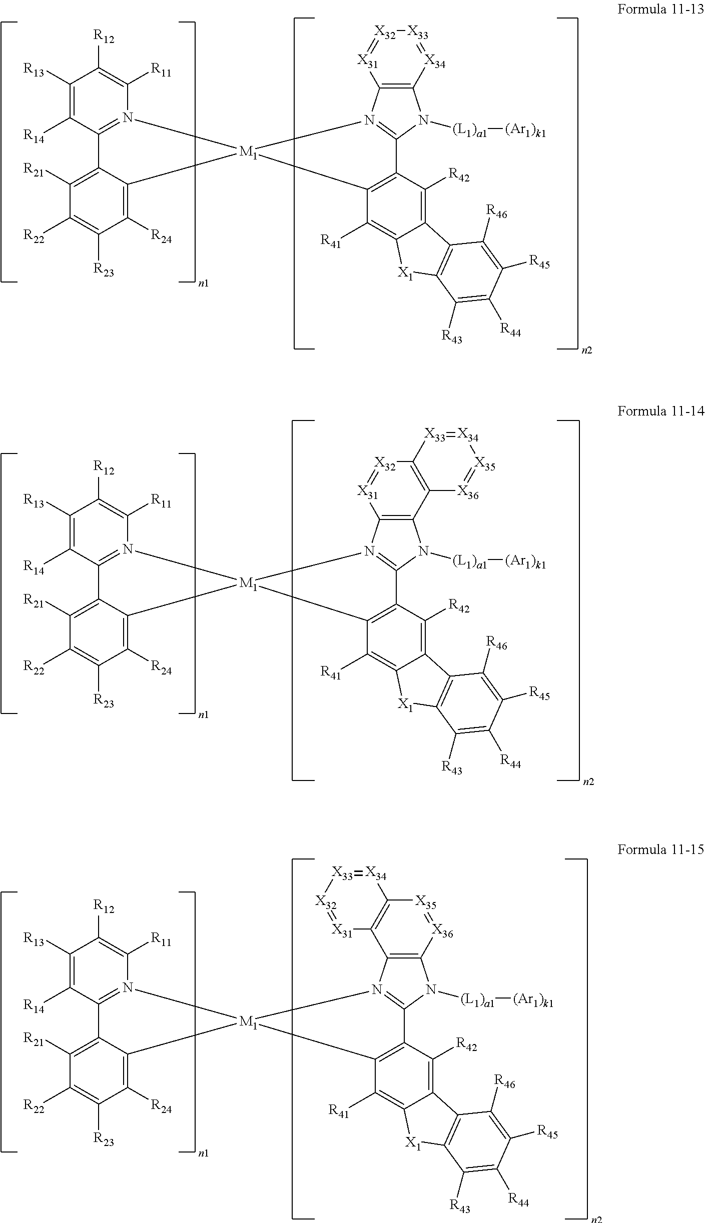 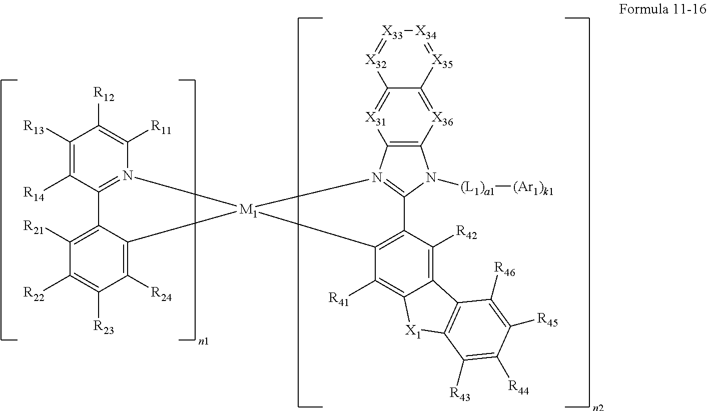 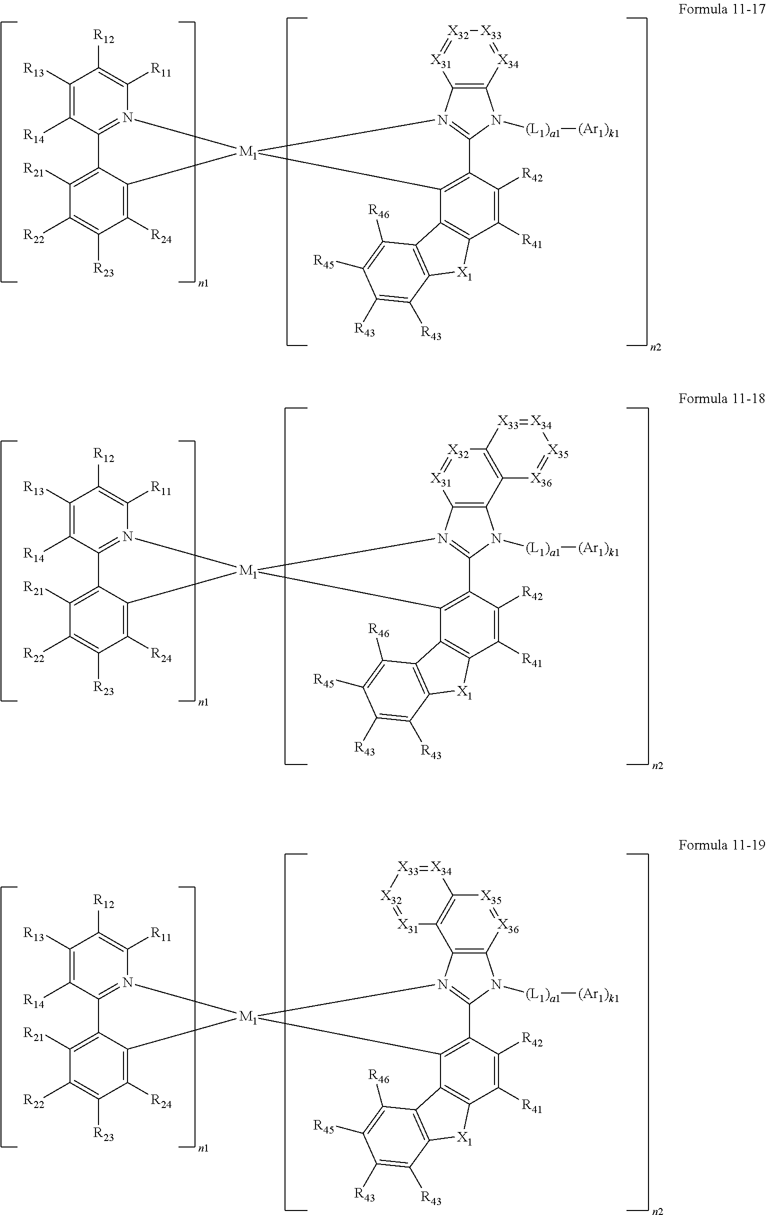 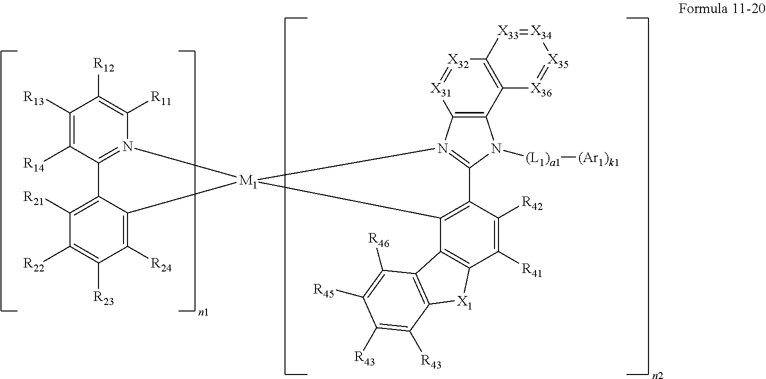 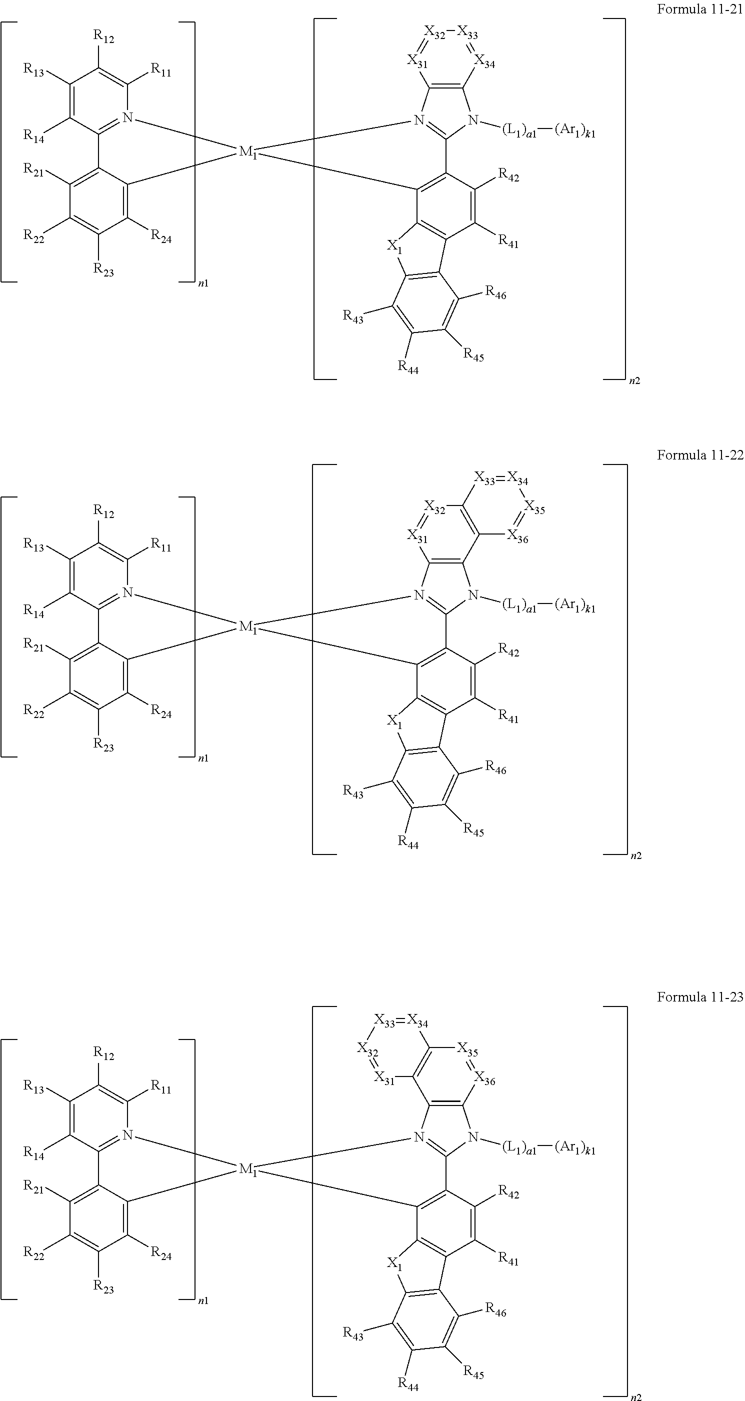 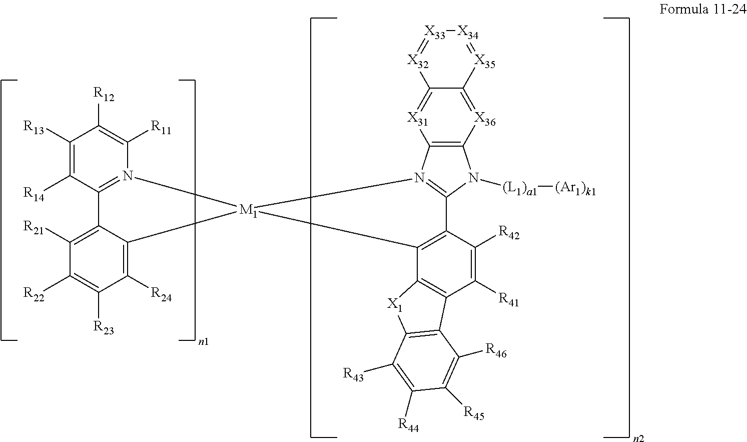
wherein, in Formulae 11-1 to 11-24,
M₁, n1, n2, X₁, L₁, a1, Ar₁, and k1 are each as defined in claim 1,
X₃₁ is C(R₃₁) or N, X₃₂ is C(R₃₂) or N, X₃₃ is C(R₃₃) or N, X₃₄ is C(R₃₄) or N, X₃₅ is C(R₃₅) or N, and X₃₆ is C(R₃₆) or N,
R₁₁ to R₁₄ are each independently as described in connection with R₁₀ in claim 1,
R₂₁ to R₂₄ are each independently as described in connection with R₂₀ in claim 1,
R₃₁ to R₃₆ are each independently as described in connection with R₃₀ in claim 1,
two or more of R₃₁ to R₃₆ are optionally linked to form a C₅-C₃₀ carbocyclic group unsubstituted or substituted with at least one R₁₀ₐ or a C₁-C₃₀ heterocyclic group unsubstituted or substituted with at least one R₁₀ₐ,
R₄₁ to R₄₆ are each independently as described in connection with R₄₀ in claim 1, and
at least one of Ar₁, R₁₁ to R₁₄, R₂₁ to R₂₄, R₃₁ to R₃₆, and R₄₁ to R₄₆ includes —F.

6. The organometallic compound of claim 1, wherein CY₁ is a benzene group, a naphthalene group, a 1,2,3,4-tetrahydronaphthalene group, a phenanthrene group, a pyridine group, a pyrimidine group, a pyrazine group, a triazine group, a quinoline group, an isoquinoline group, a quinoxaline group, a quinazoline group, a phenanthroline group, a benzofuran group, a benzothiophene group, a fluorene group, a carbazole group, a dibenzofuran group, a dibenzothiophene group, a dibenzosilole group, an azafluorene group, an azacarbazole group, an azadibenzofuran group, an azadibenzothiophene group, or an azadibenzosilole group.

7. The organometallic compound of claim 1, wherein L₁ is: a phenylene group, a naphthylene group, a fluorenylene group, a spiro-bifluorenylene group, a benzofluorenylene group, a dibenzofluorenylene group, a phenanthrenylene group, an anthracenylene group, a fluoranthenylene group, a triphenylenylene group, a pyrenylene group, a chrysenylene group, a perylenylene group, a thiophenylene group, a furanylene group, a carbazolylene group, an indolylene group, an isoindolylene group, a benzofuranylene group, a benzothiophenylene group, a dibenzofuranylene group, a dibenzothiophenylene group, a benzocarbazolylene group, a dibenzocarbazolylene group, a dibenzosilolylene group, a pyridinylene group, an imidazolylene group, a pyrazolylene group, a thiazolylene group, an isothiazolylene group, an oxazolylene group, an isoxazolylene group, a thiadiazolylene group, an oxadiazolylene group, a pyrazinylene group, a pyrimidinylene group, a pyridazinylene group, a triazinylene group, a quinolinylene group, an isoquinolinylene group, a benzoquinolinylene group, a phthalazinylene group, a naphthyridinylene group, a quinoxalinylene group, a quinazolinylene group, a cinnolinylene group, a phenanthridinylene group, an acridinylene group, a phenanthrolinylene group, a phenazinylene group, a benzimidazolylene group, an isobenzothiazolylene group, a benzoxazolylene group, an isobenzoxazolylene group, a triazolylene group, a tetrazolylene group, an imidazopyridinylene group, an imidazopyrimidinylene group, or an azacarbazolylene group; or
a phenylene group, a naphthylene group, a fluorenylene group, a spiro-bifluorenylene group, a benzofluorenylene group, a dibenzofluorenylene group, a phenanthrenylene group, an anthracenylene group, a fluoranthenylene group, a triphenylenylene group, a pyrenylene group, a chrysenylene group, a perylenylene group, a thiophenylene group, a furanylene group, a carbazolylene group, an indolylene group, an isoindolylene group, a benzofuranylene group, a benzothiophenylene group, a dibenzofuranylene group, a dibenzothiophenylene group, a benzocarbazolylene group, a dibenzocarbazolylene group, a dibenzosilolylene group, a pyridinylene group, an imidazolylene group, a pyrazolylene group, a thiazolylene group, an isothiazolylene group, an oxazolylene group, an isoxazolylene group, a thiadiazolylene group, an oxadiazolylene group, a pyrazinylene group, a pyrimidinylene group, a pyridazinylene group, a triazinylene group, a quinolinylene group, an isoquinolinylene group, a benzoquinolinylene group, a phthalazinylene group, a naphthyridinylene group, a quinoxalinylene group, a quinazolinylene group, a cinnolinylene group, a phenanthridinylene group, an acridinylene group, a phenanthrolinylene group, a phenazinylene group, a benzimidazolylene group, a benzoisothiazolylene group, a benzoxazolylene group, an isobenzoxazolylene group, a triazolylene group, a tetrazolylene group, an imidazopyridinylene group, an imidazopyrimidinylene group, or an azacarbazolylene group, each substituted with deuterium, —F, —Cl, —Br, —I, —CD₃, —CD₂H, —CDH₂, —CF₃, —CF₂H, —CFH₂, a hydroxyl group, a cyano group, a nitro group, an amidino group, a hydrazino group, a hydrazono group, a C₁-C₂₀ alkyl group, a C₁-C₂₀ alkoxy group, a phenyl group, a biphenyl group, a terphenyl group, a naphthyl group, —Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), —P(═O)(Q₃₁)(Q₃₂), —P(Q₃₁)(Q₃₂), or a combination thereof.

8. The organometallic compound of claim 1, wherein Ar₁, R₁₀, R₂₀, R₃₀, and R₄₀ are each independently:
hydrogen, deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, —SF₅, a C₁-C₂₀ alkyl group, or a C₁-C₂₀ alkoxy group;
a C₁-C₂₀ alkyl group or a C₁-C₂₀ alkoxy group, each substituted with deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₁₀ alkyl group, a cyclopentyl group, a cyclohexyl group, a cycloheptyl group, a cycloctyl group, an adamantanyl group, a norbornanyl group, a norbornenyl group, a cyclopentenyl group, a cyclohexenyl group, a cycloheptenyl group, a phenyl group, a naphthyl group, a pyridinyl group, a pyrimidinyl group, or a combination thereof;
a cyclopentyl group, a cyclohexyl group, a cycloheptyl group, a cycloctyl group, an adamantanyl group, a norbornanyl group, a norbornenyl group, a cyclopentenyl group, a cyclohexenyl group, a cycloheptenyl group, a phenyl group, a naphthyl group, a fluorenyl group, a phenanthrenyl group, an anthracenyl group, a fluoranthenyl group, a triphenylenyl group, a pyrenyl group, a chrysenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a quinoxalinyl group, a quinazolinyl group, a cinnolinyl group, a carbazolyl group, a phenanthrolinyl group, a benzimidazolyl group, a benzofuranyl group, a benzothiophenyl group, an isobenzothiazolyl group, a benzoxazolyl group, an isobenzoxazolyl group, a triazolyl group, a tetrazolyl group, an oxadiazolyl group, a triazinyl group, a dibenzofuranyl group, a dibenzothiophenyl group, a benzocarbazolyl group, a dibenzocarbazolyl group, an imidazopyridinyl group, or an imidazopyrimidinyl group;
a cyclopentyl group, a cyclohexyl group, a cycloheptyl group, a cycloctyl group, an adamantanyl group, a norbornanyl group, a norbornenyl group, a cyclopentenyl group, a cyclohexenyl group, a cycloheptenyl group, a phenyl group, a naphthyl group, a fluorenyl group, a phenanthrenyl group, an anthracenyl group, a fluoranthenyl group, a triphenylenyl group, a pyrenyl group, a chrysenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a quinoxalinyl group, a quinazolinyl group, a cinnolinyl group, a carbazolyl group, a phenanthrolinyl group, a benzimidazolyl group, a benzofuranyl group, a benzothiophenyl group, an isobenzothiazolyl group, a benzoxazolyl group, an isobenzoxazolyl group, a triazolyl group, a tetrazolyl group, an oxadiazolyl group, a triazinyl group, a dibenzofuranyl group, a dibenzothiophenyl group, a benzocarbazolyl group, a dibenzocarbazolyl group, an imidazopyridinyl group, or an imidazopyrimidinyl group, each substituted with deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₂₀ alkyl group, a C₁-C₂₀ alkoxy group, a cyclopentyl group, a cyclohexyl group, a cycloheptyl group, a cyclooctyl group, an adamantanyl group, a norbornanyl group, a norbornenyl group, a cyclopentenyl group, a cyclohexenyl group, a cycloheptenyl group, a phenyl group, a naphthyl group, a fluorenyl group, a phenanthrenyl group, an anthracenyl group, a fluoranthenyl group, a triphenylenyl group, a pyrenyl group, a chrysenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a quinoxalinyl group, a quinazolinyl group, a cinnolinyl group, a carbazolyl group, a phenanthrolinyl group, a benzimidazolyl group, a benzofuranyl group, a benzothiophenyl group, an isobenzothiazolyl group, a benzoxazolyl group, an isobenzoxazolyl group, a triazolyl group, a tetrazolyl group, an oxadiazolyl group, a triazinyl group, a dibenzofuranyl group, a dibenzothiophenyl group, a benzocarbazolyl group, a dibenzocarbazolyl group, an imidazopyridinyl group, an imidazopyrimidinyl group, or a combination thereof; or
—N(Q₁)(Q₂), —Si(Q₃)(Q₄)(Q₅), —Ge(Q₃)(Q₄)(Q₅), —B(Q₆)(Q₇), —C(═O)(Q₆), —S(═O)₂(Q₆), —P(═O)(Q₈)(Q₉), or —P(Q₈)(Q₉),
wherein Q₁ to Q₉ are each independently:
—CH₃, —CD₃, —CD₂H, —CDH₂, —CH₂CH₃, —CH₂CD₃, —CH₂CD₂H, —CH₂CDH₂, —CHDCH₃, —CHDCD₂H, —CHDCDH₂, —CHDCD₃, —CD₂CD₃, —CD₂CD₂H, or —CD₂CDH₂;
an n-propyl group, an isopropyl group, an n-butyl group, an isobutyl group, a sec-butyl group, a tert-butyl group, an n-pentyl group, an isopentyl group, a sec-pentyl group, a tert-pentyl group, a phenyl group, or a naphthyl group; or
an n-propyl group, an isopropyl group, an n-butyl group, an isobutyl group, a sec-butyl group, a tert-butyl group, an n-pentyl group, an isopentyl group, a sec-pentyl group, a tert-pentyl group, a phenyl group, or a naphthyl group, each substituted with deuterium, a C₁-C₁₀ alkyl group, a phenyl group, or a combination thereof.

9. The organometallic compound of claim 1, wherein Ar₁, R₁₀, R₂₀, R₃₀, and R₄₀ are each independently:
hydrogen, deuterium, —F, —Cl, —Br, —I, —CD₃, —CD₂H, —CDH₂, —CF₃, —CF₂H, —CFH₂, a C₁-C₆₀ alkyl group, a C₂-C₆₀ alkenyl group, a C₂-C₆₀ alkynyl group, or a C₁-C₆₀ alkoxy group; or
a group represented by one of Formulae 9-1 to 9-67, 9-201 to 9-244, 10-1 to 10-154, and 10-201 to 10-350: 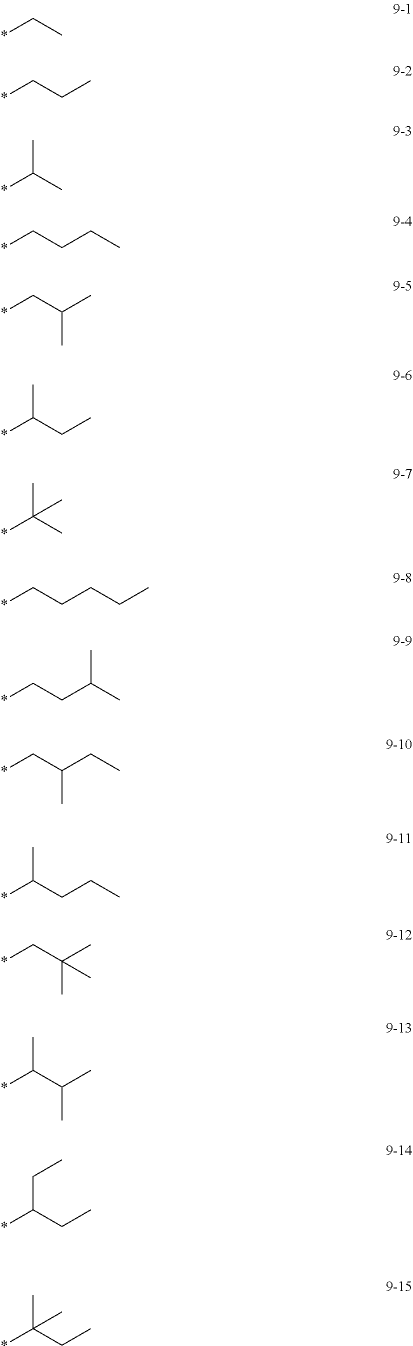 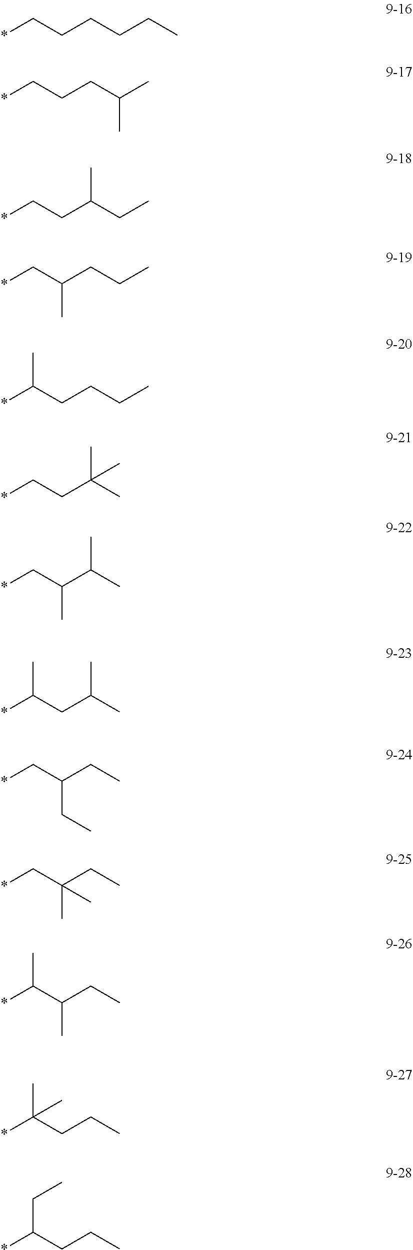 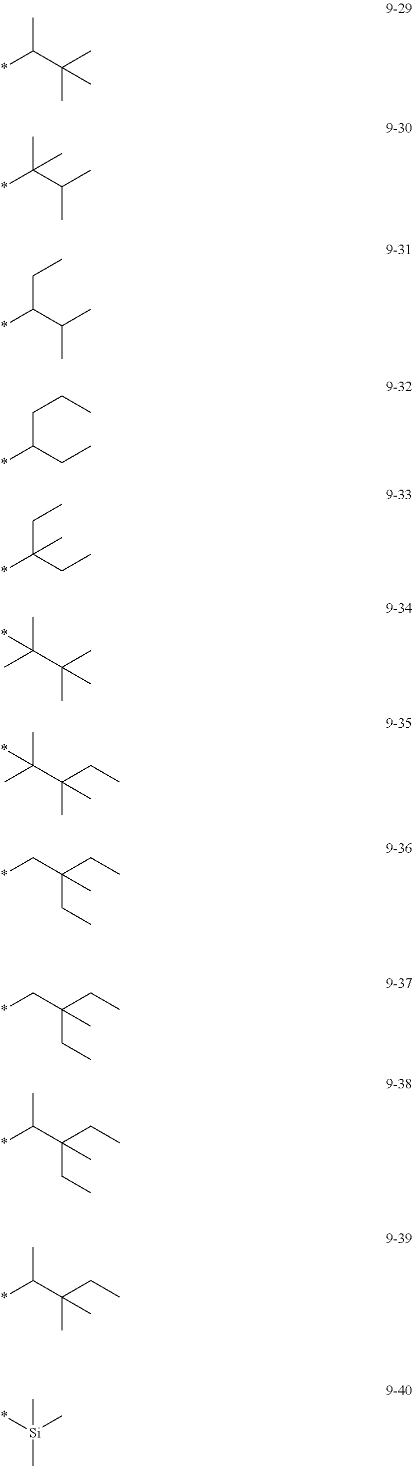 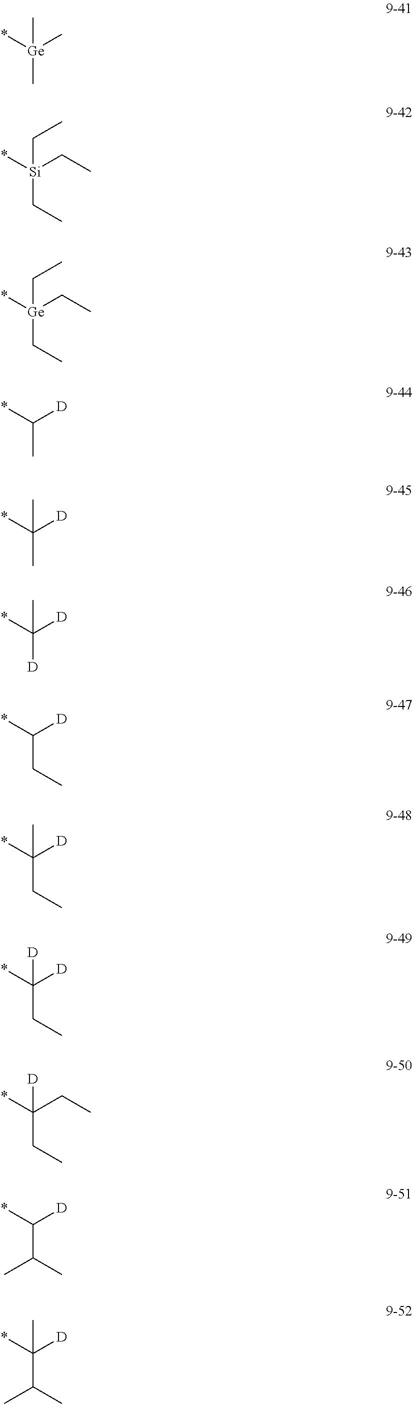 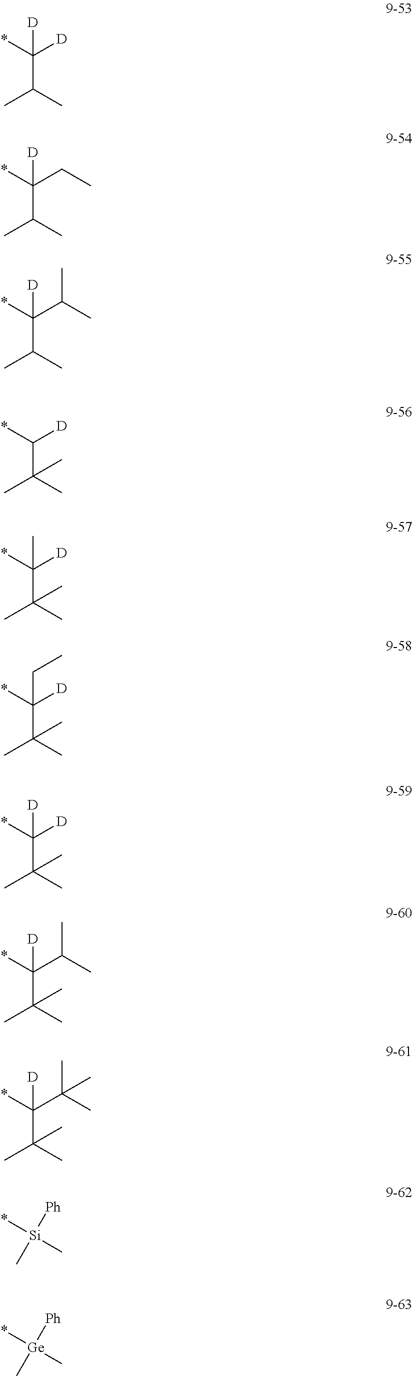 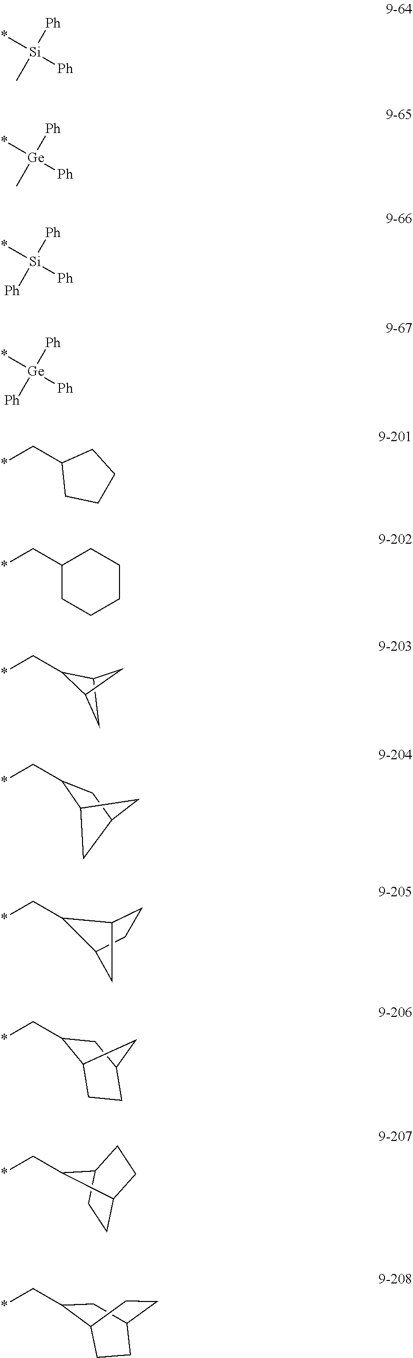 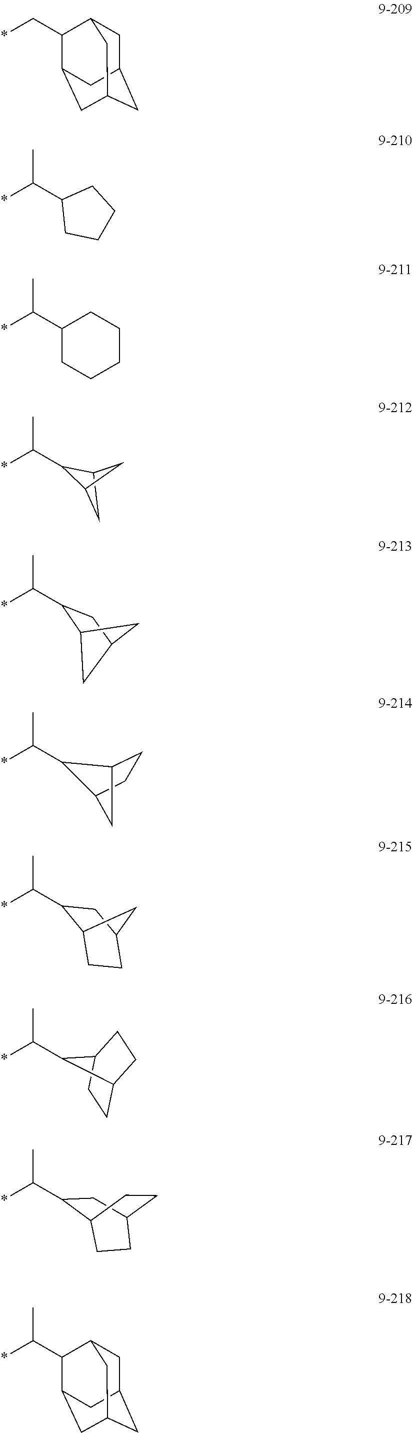 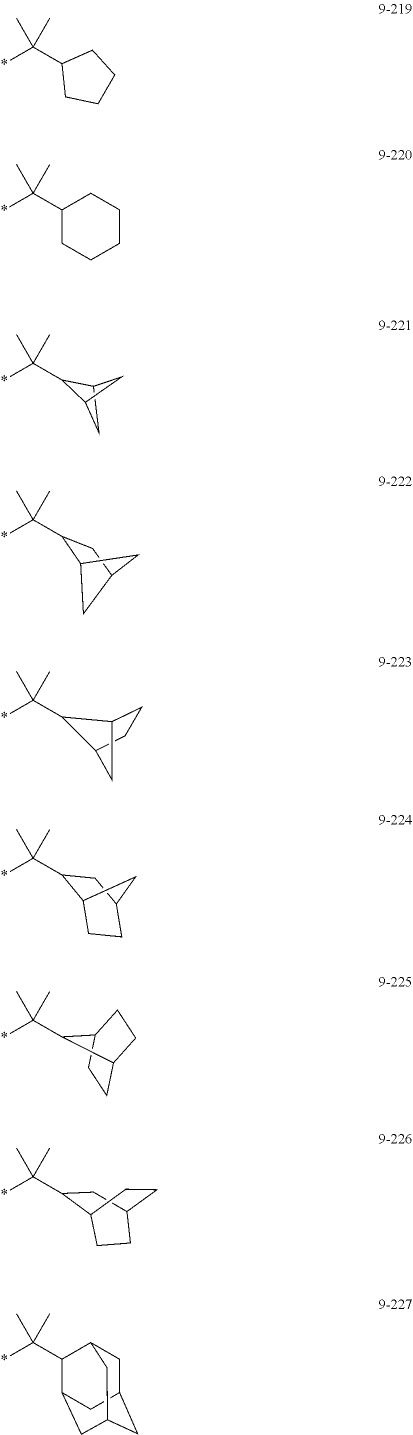 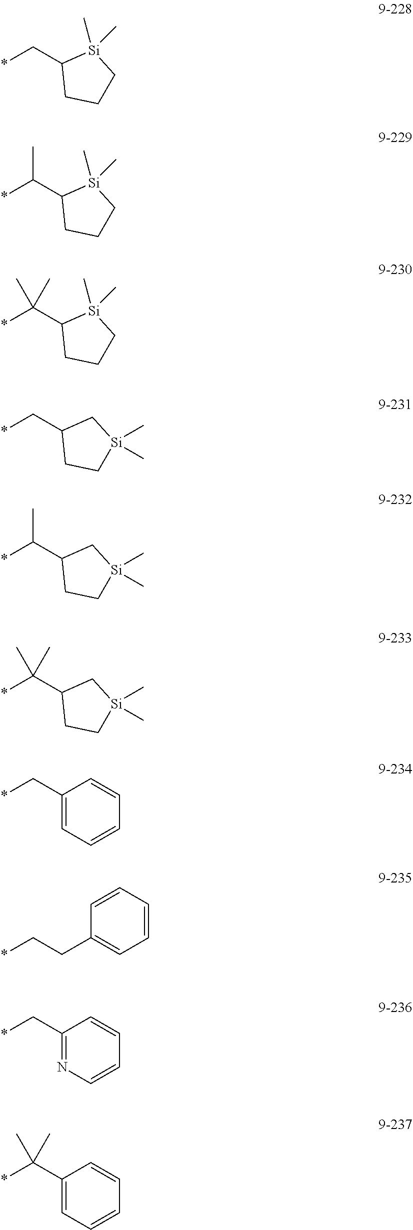 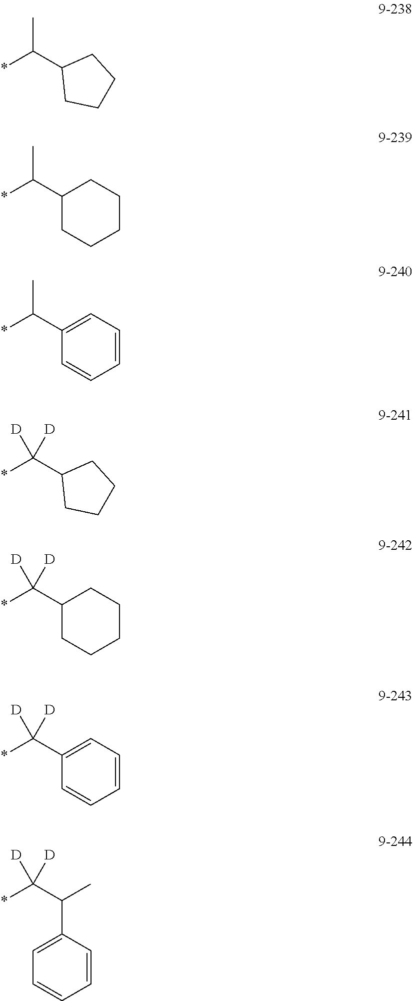 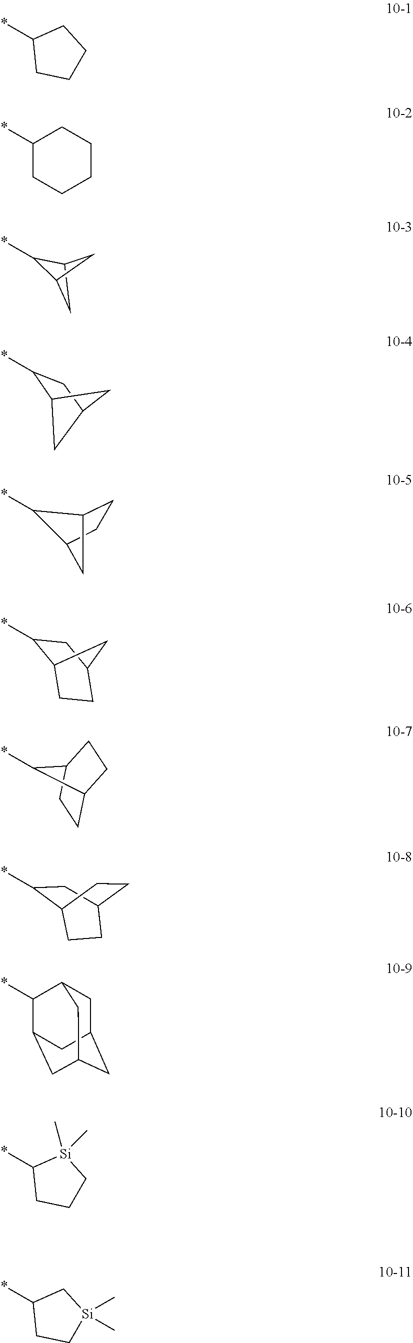 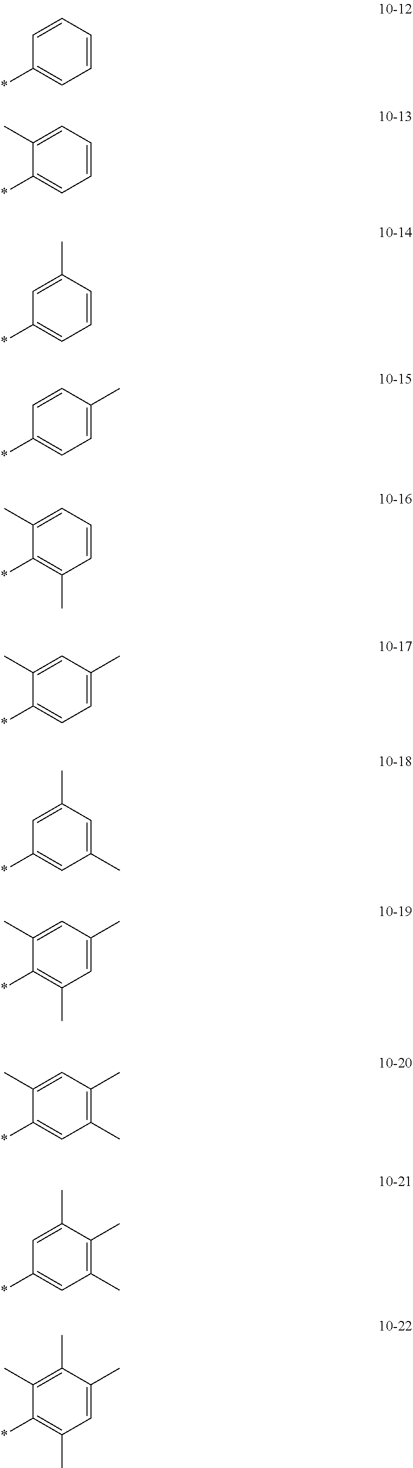 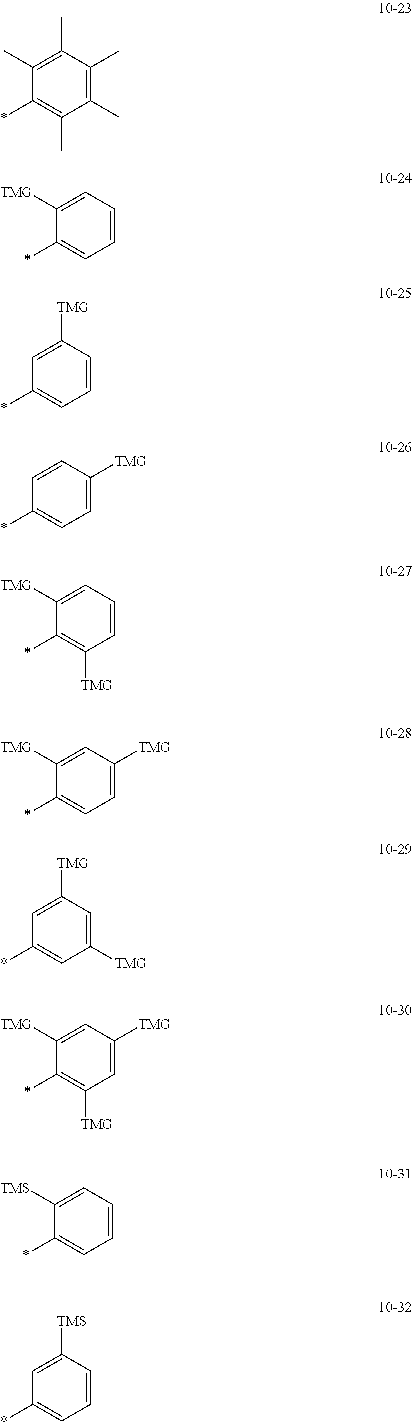 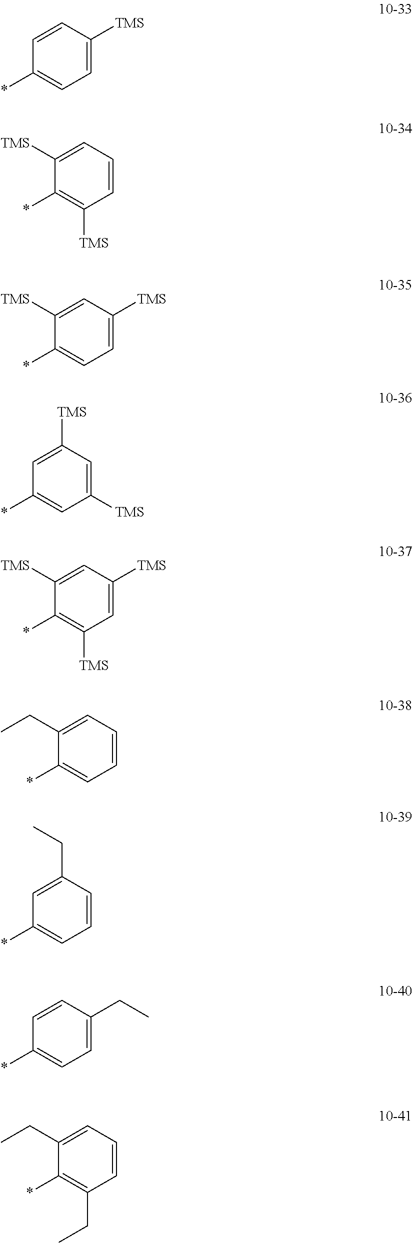 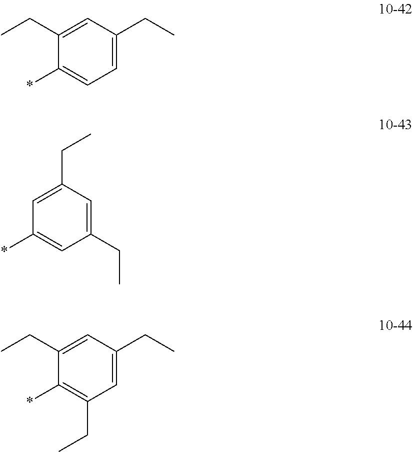 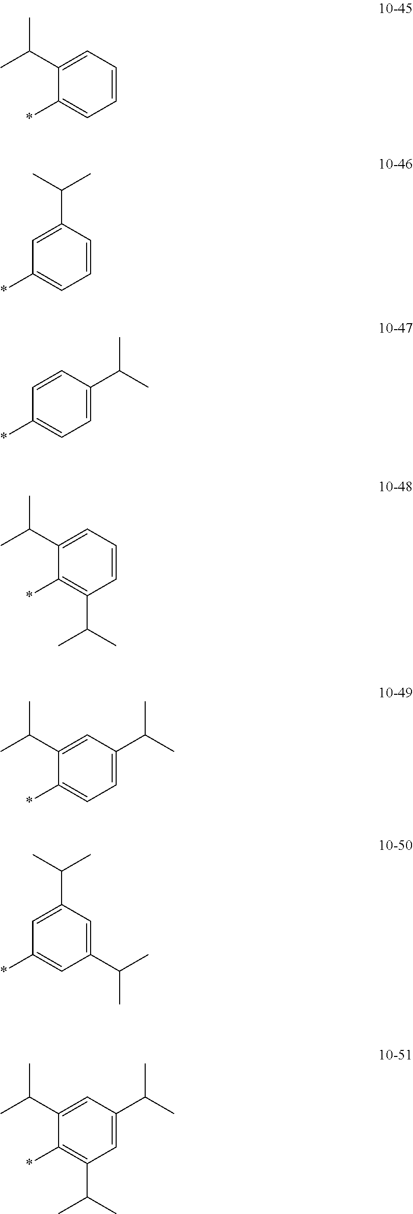 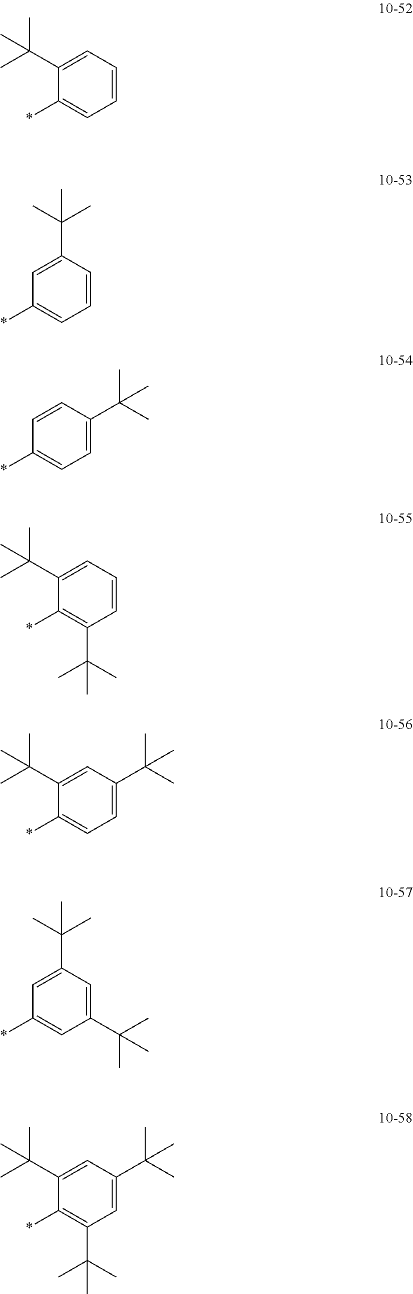 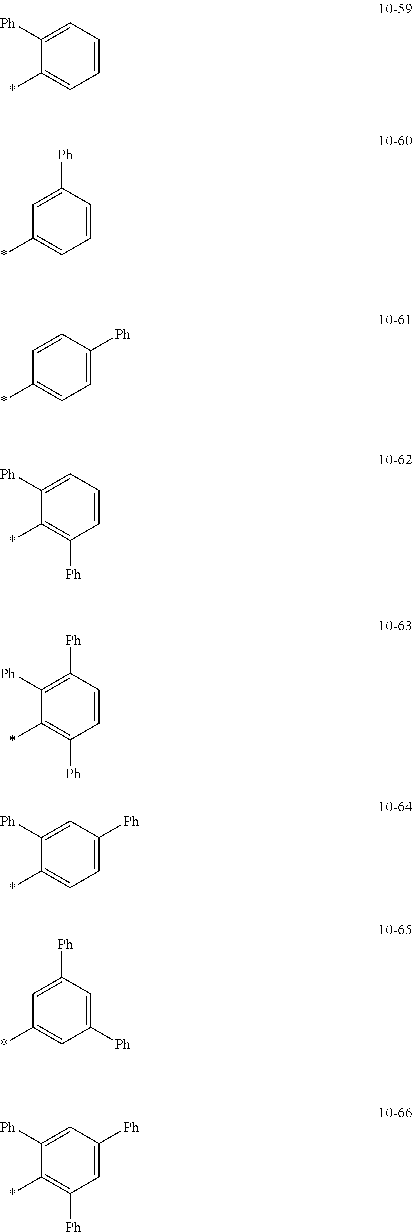 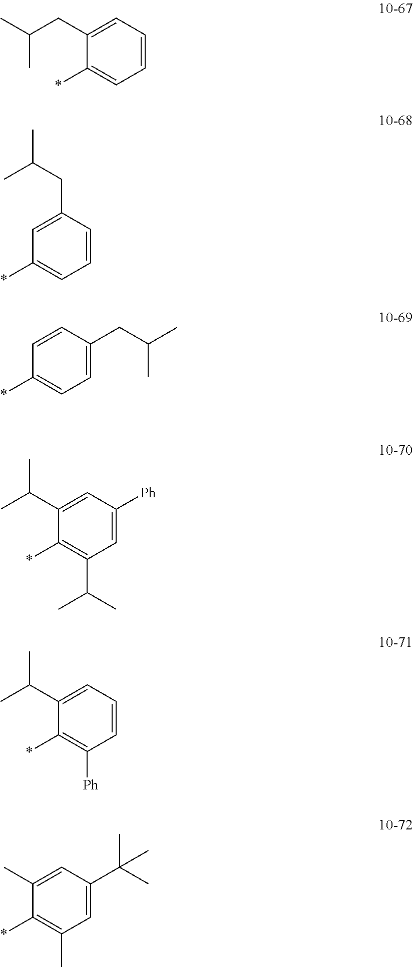 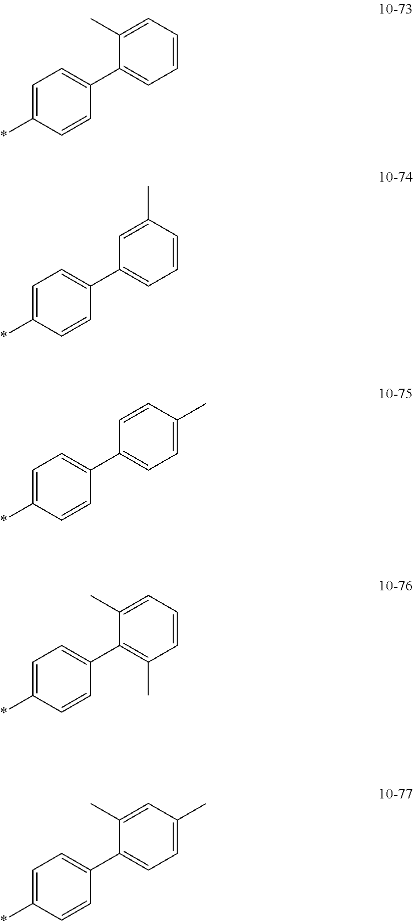 [SMILES: *c1ccc(-c2ccccc2C)cc1 ; *c1ccc(-c2cccc(C)c2)cc1 ; *c1ccc(-c2ccc(C)cc2)cc1 ; *c1ccc(-c2c(C)cccc2C)cc1 ; *c1ccc(-c2ccc(C)cc2C)cc1] 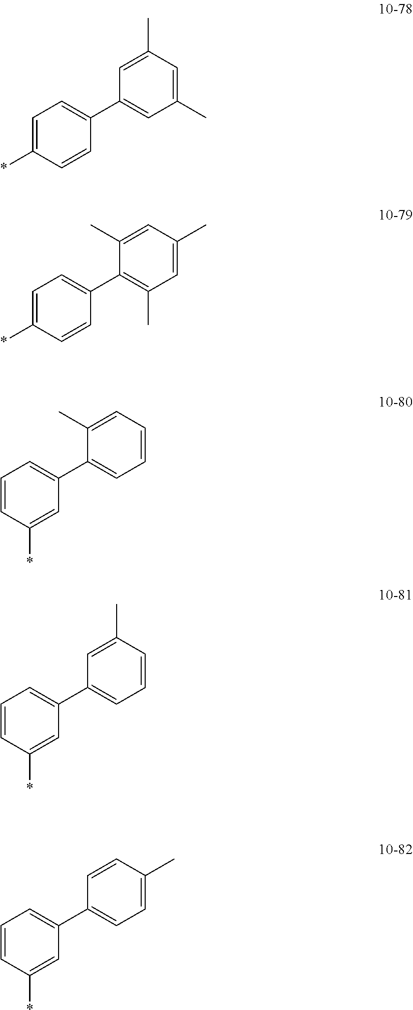 [SMILES: *c1ccc(-c2cc(C)cc(C)c2)cc1 ; *c1ccc(-c2c(C)cc(C)cc2C)cc1 ; *c1cccc(-c2ccccc2C)c1 ; *c1cccc(-c2cccc(C)c2)c1 ; *c1cccc(-c2ccc(C)cc2)c1] 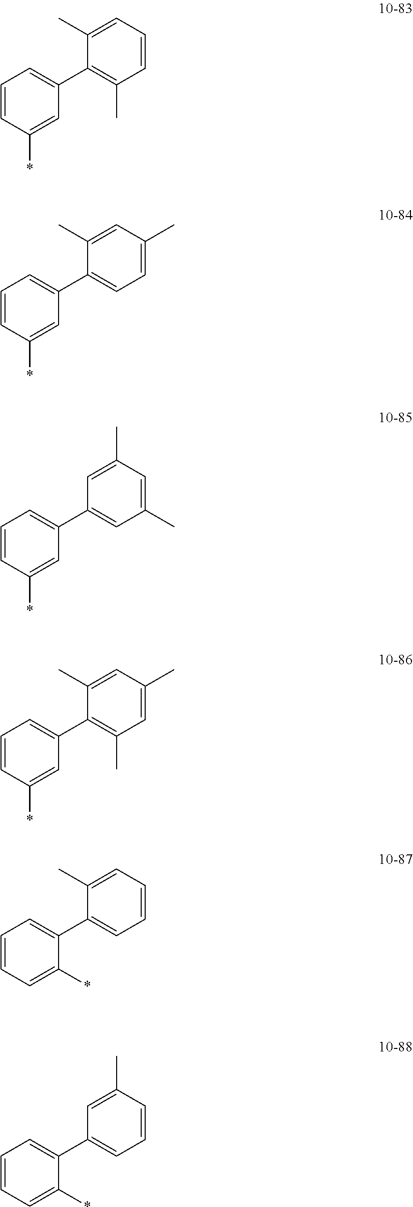 [SMILES: *c1cccc(-c2c(C)cccc2C)c1 ; *c1cccc(-c2ccc(C)cc2C)c1 ; *c1cccc(-c2cc(C)cc(C)c2)c1 ; *c1cccc(-c2c(C)cc(C)cc2C)c1 ; *c1ccccc1-c1ccccc1C ; *c1ccccc1-c1cccc(C)c1] 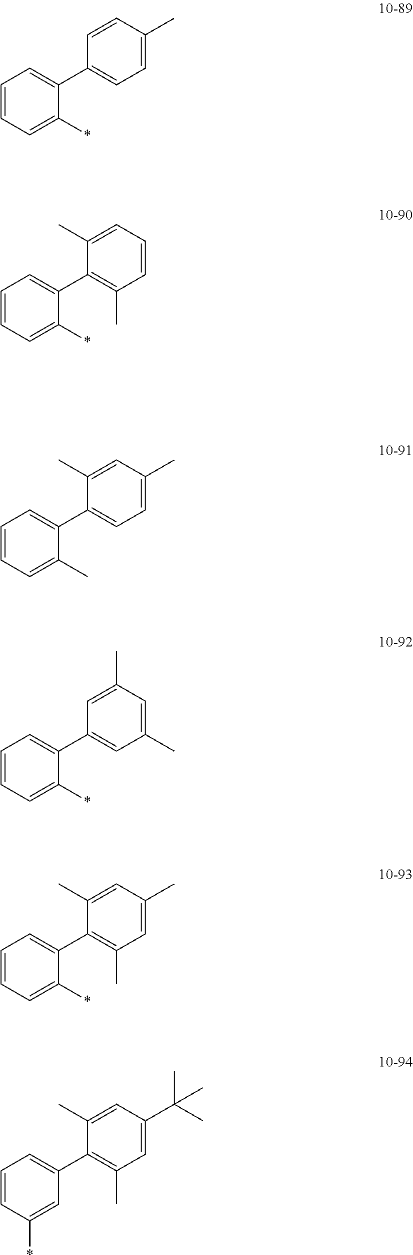 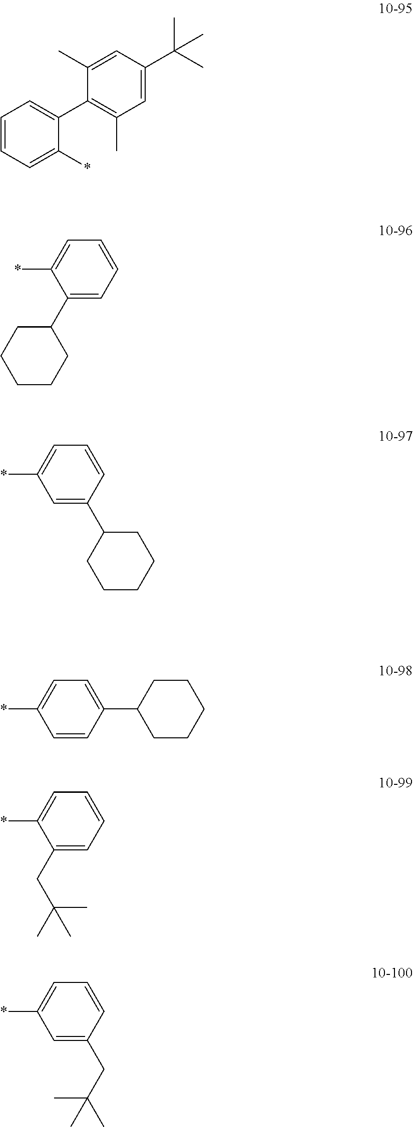 [SMILES: *c1ccccc1-c1c(C)cc(C(C)(C)C)cc1C ; *c1ccccc1C1CCCCC1 ; *c1cccc(C2CCCCC2)c1 ; *c1ccc(C2CCCCC2)cc1 ; *c1ccccc1CC(C)(C)C ; *c1cccc(CC(C)(C)C)c1] 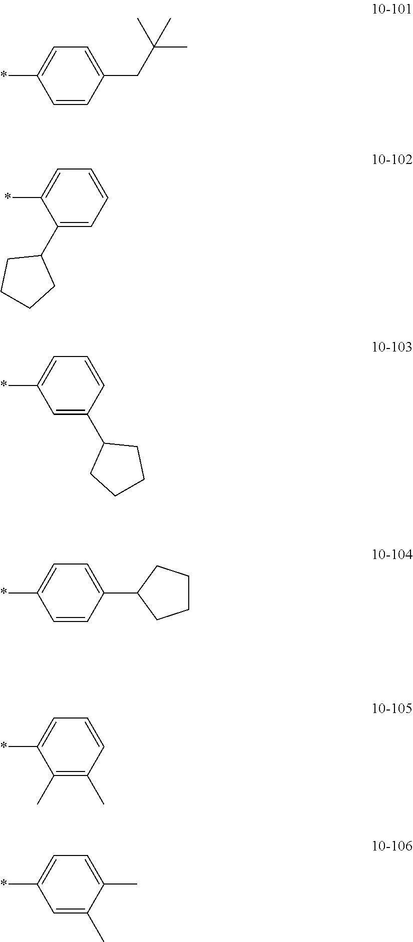 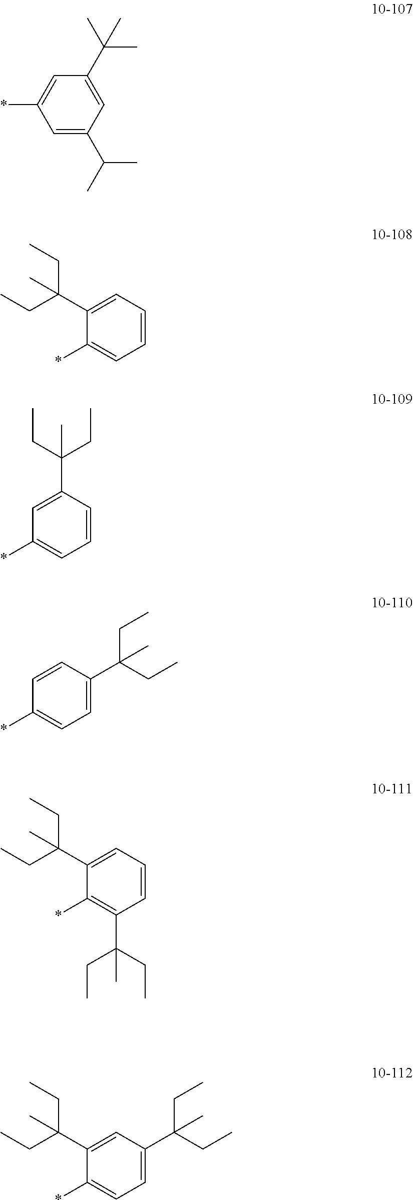 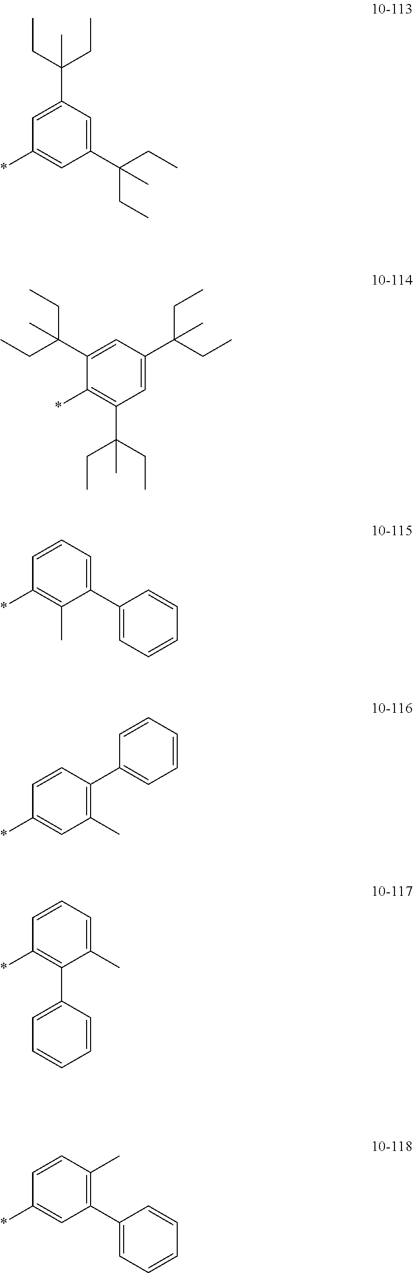 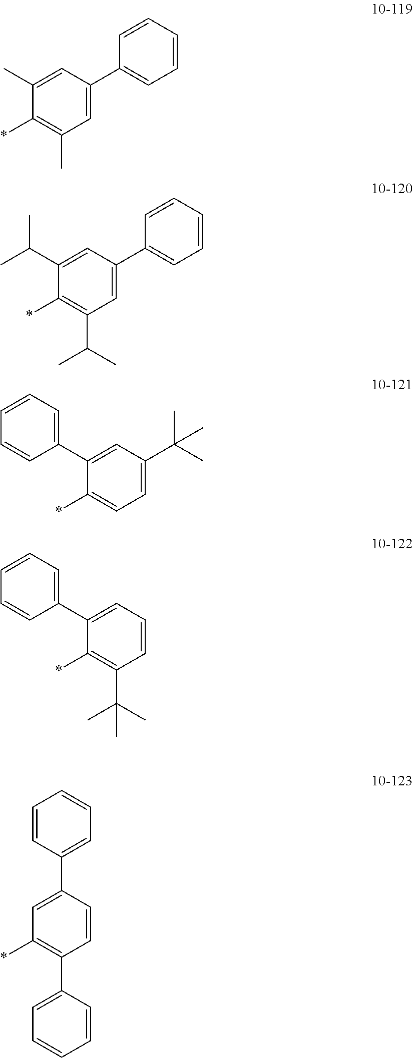 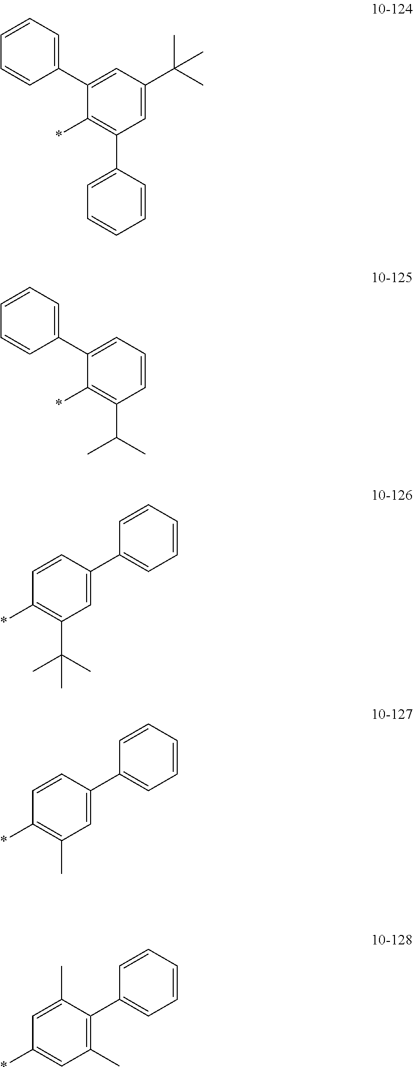 [SMILES: *c1c(-c2ccccc2)cc(C(C)(C)C)cc1-c1ccccc1 ; *c1c(-c2ccccc2)cccc1C(C)C ; *c1ccc(-c2ccccc2)cc1C(C)(C)C ; *c1ccc(-c2ccccc2)cc1C ; *c1cc(C)c(-c2ccccc2)c(C)c1] 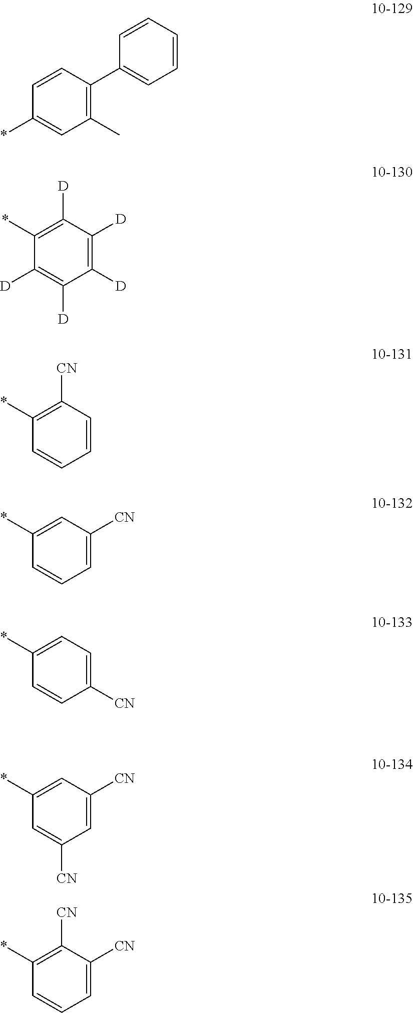 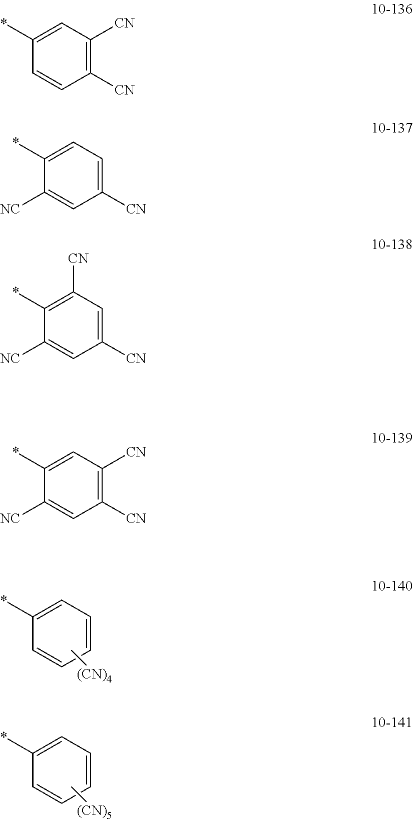 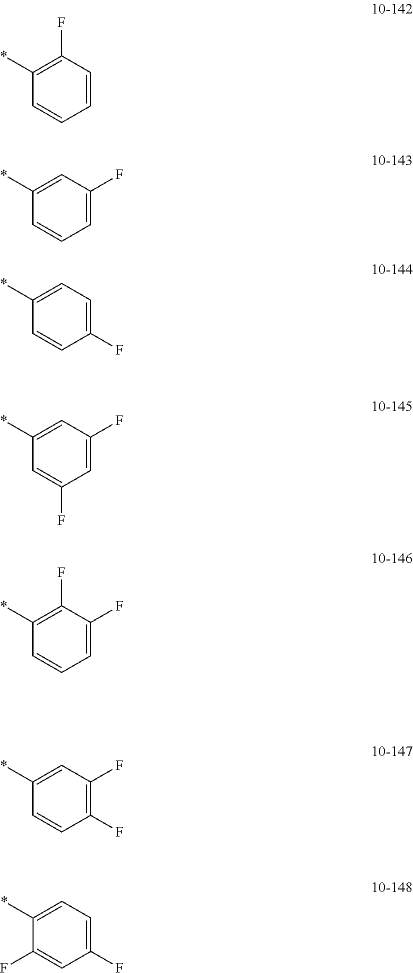 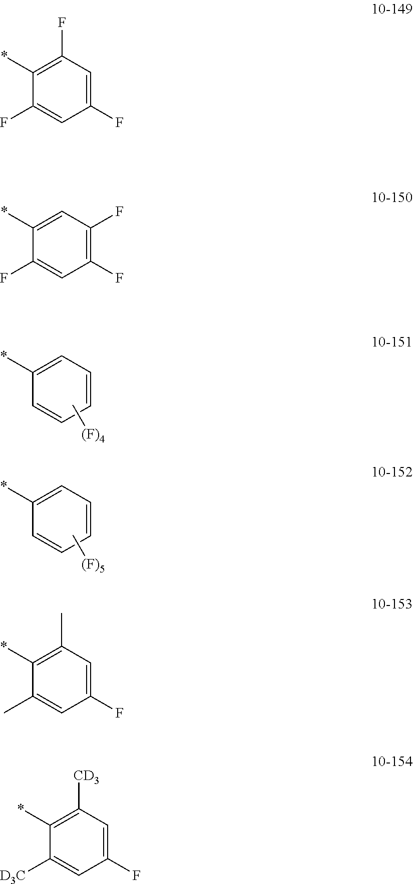 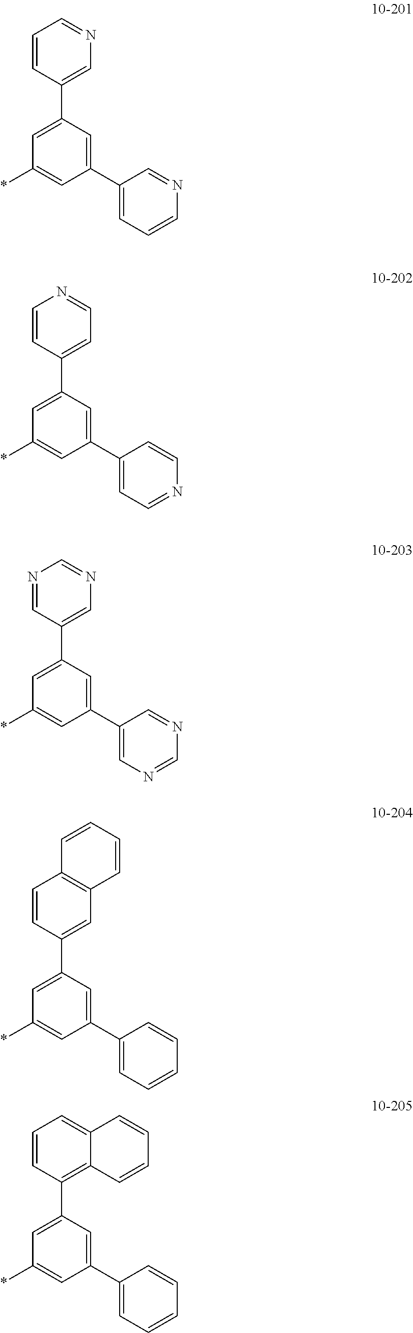 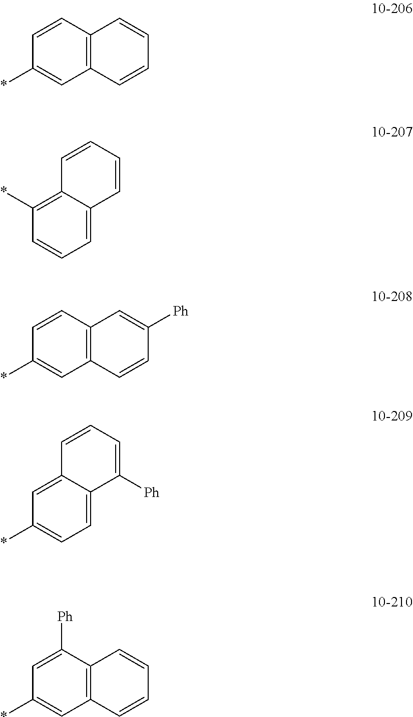 [SMILES: *c1ccc2ccccc2c1 ; *c1cccc2ccccc12 ; *c1ccc2cc(-c3ccccc3)ccc2c1 ; *c1ccc2c(-c3ccccc3)cccc2c1 ; *c1cc(-c2ccccc2)c2ccccc2c1] 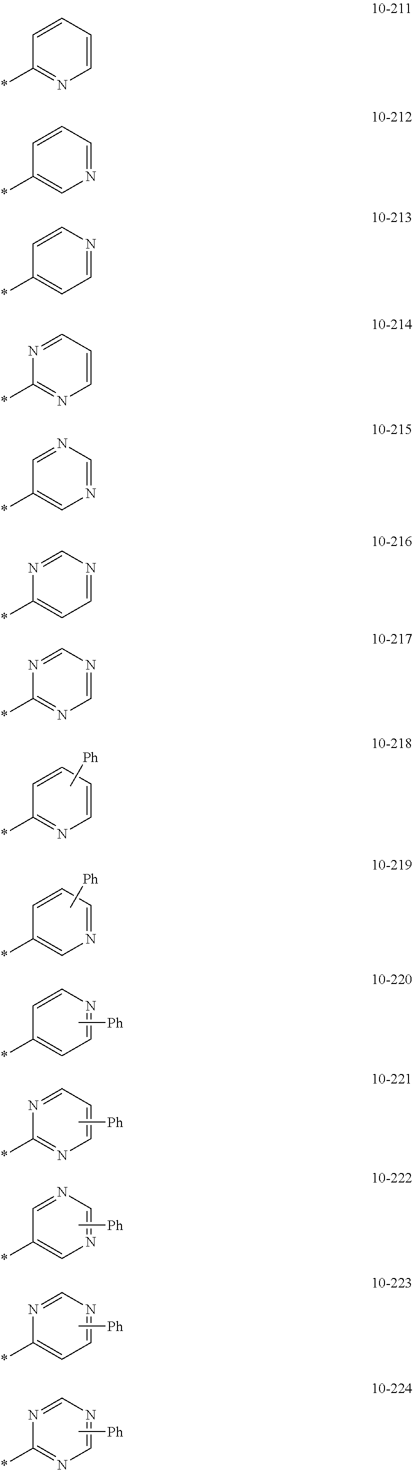 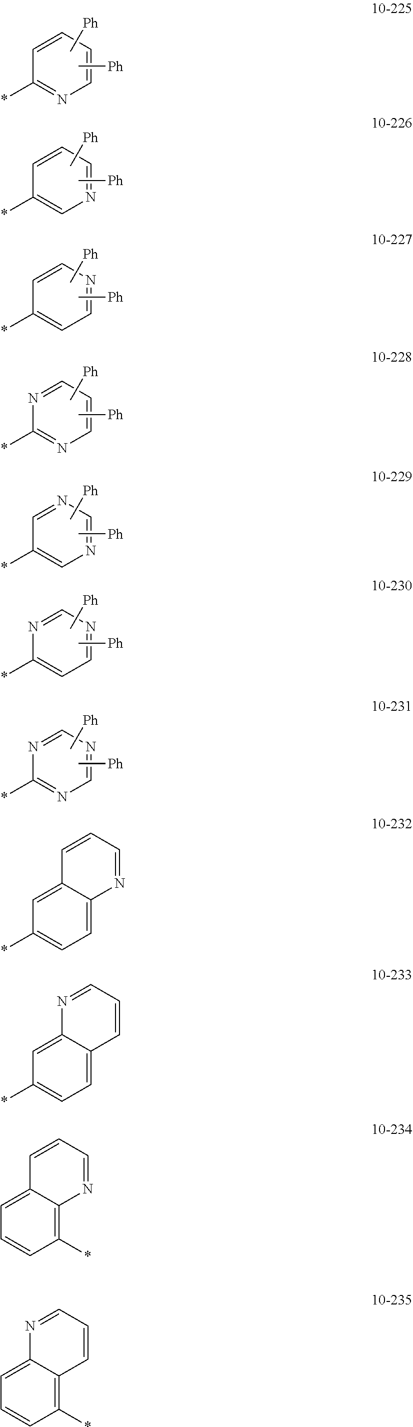 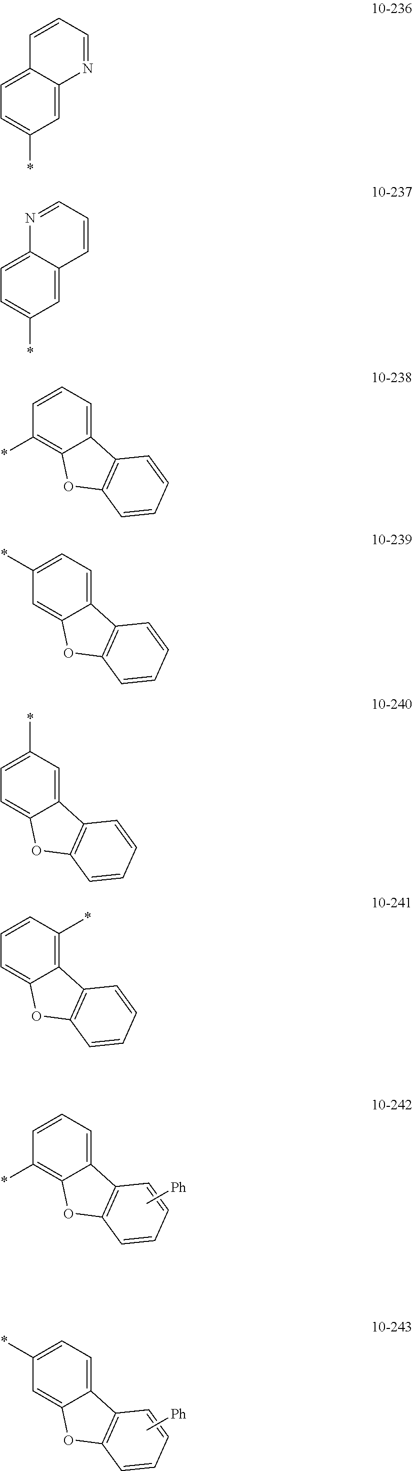 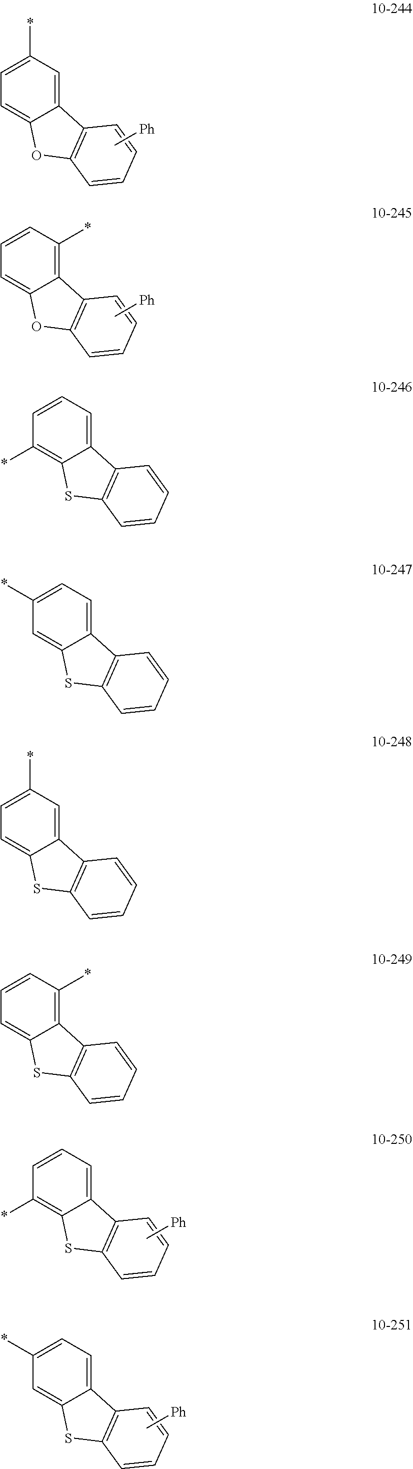 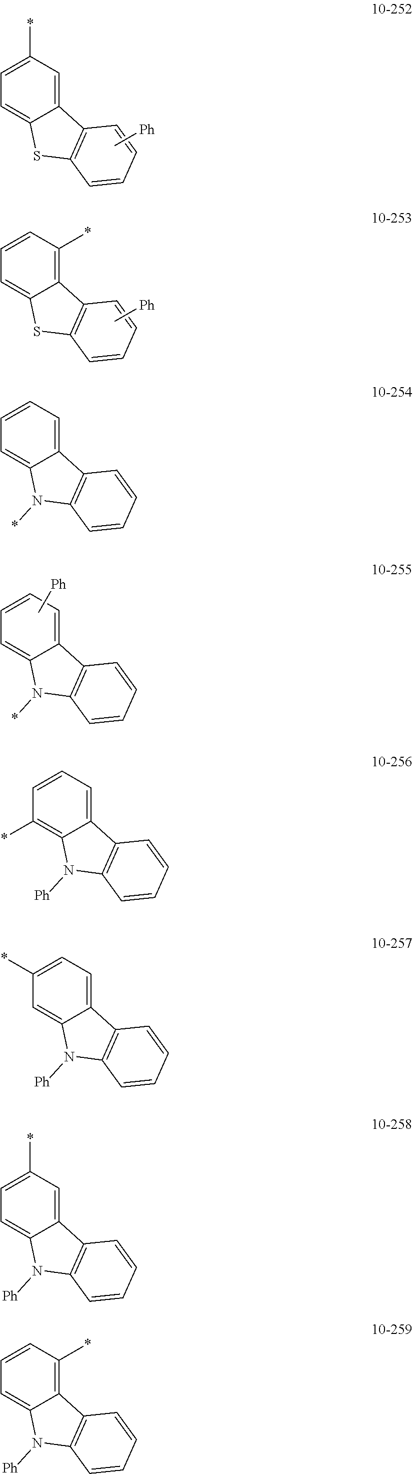 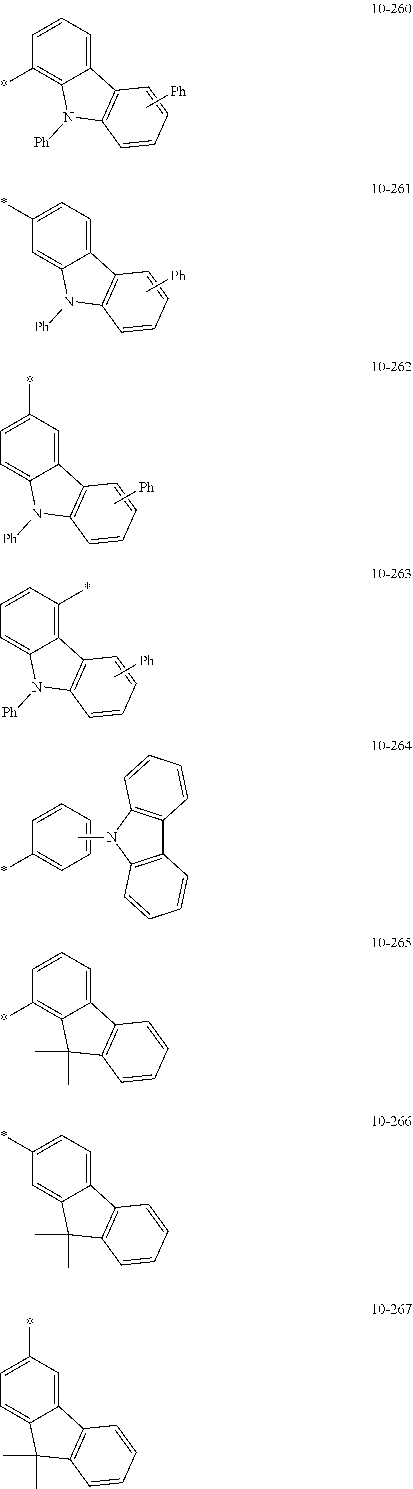 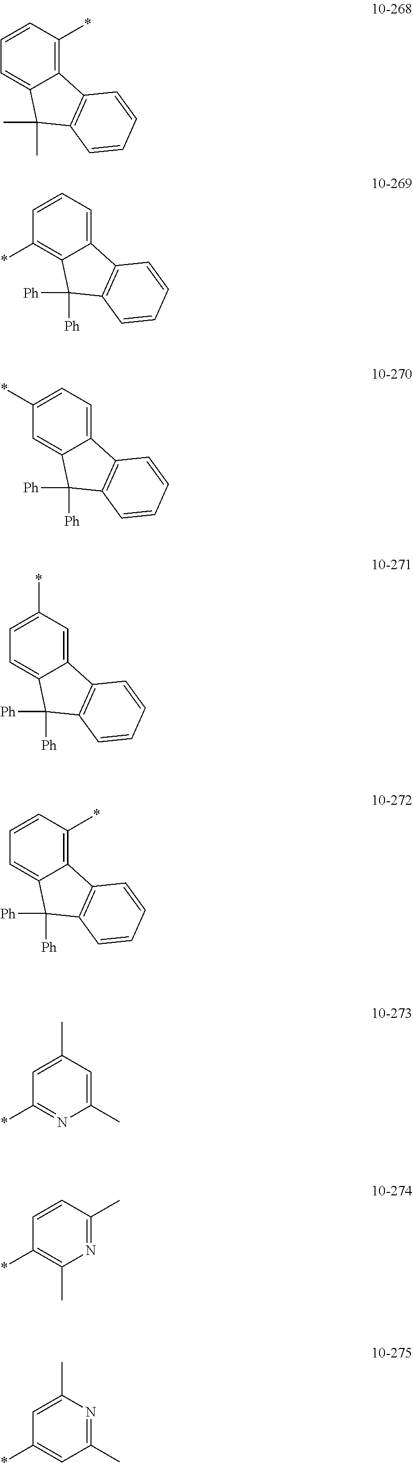 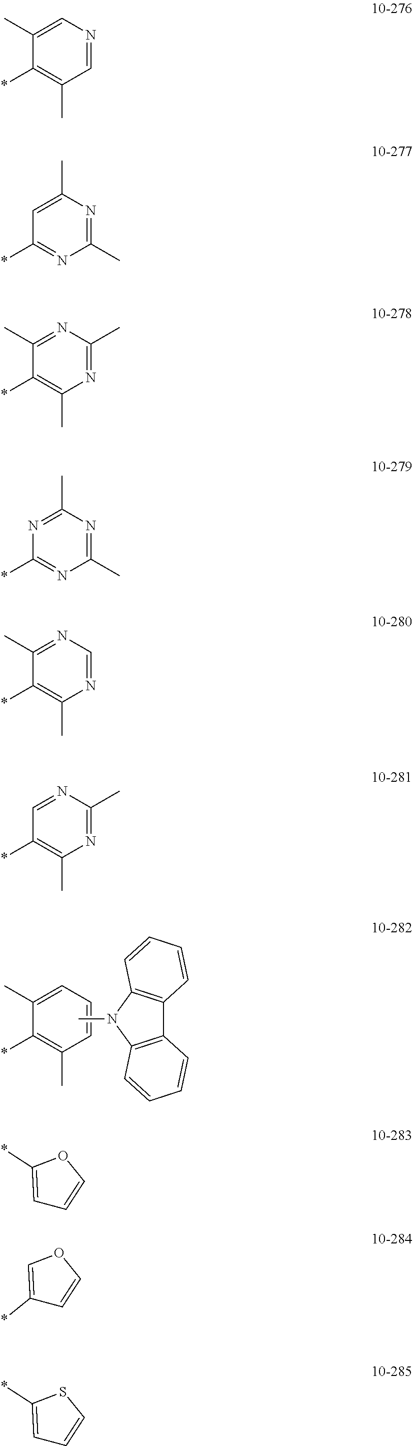 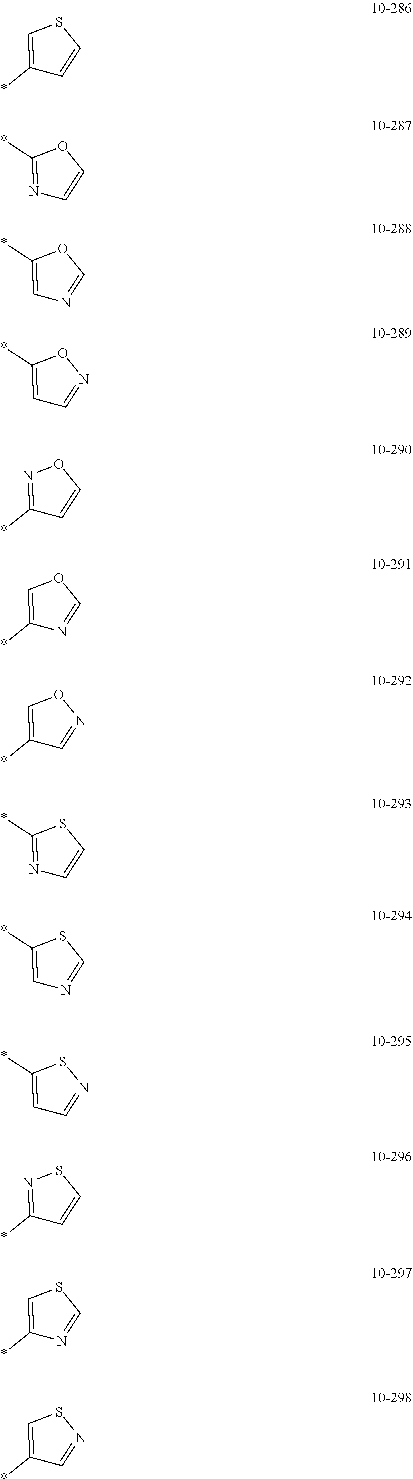 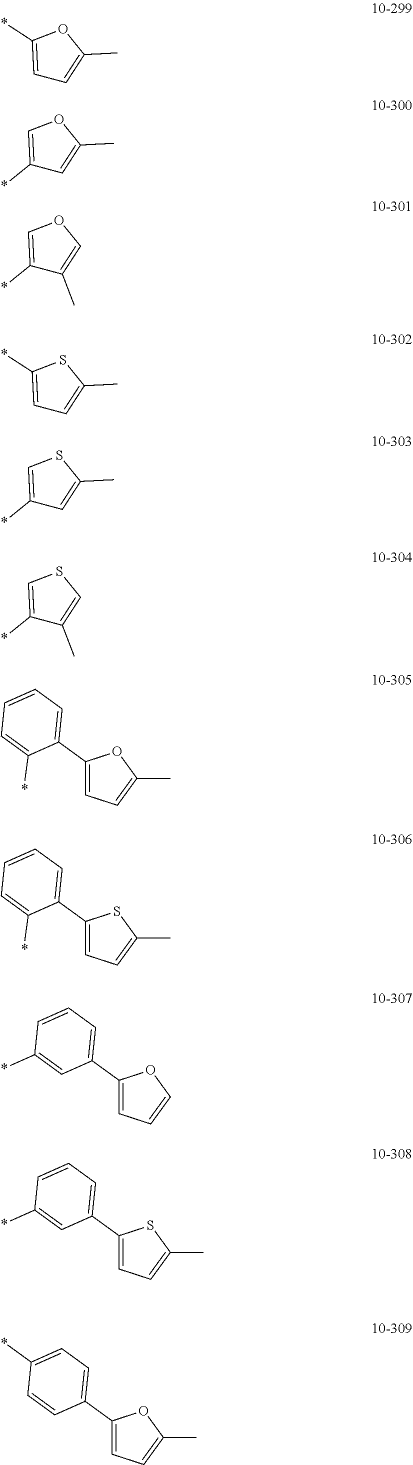 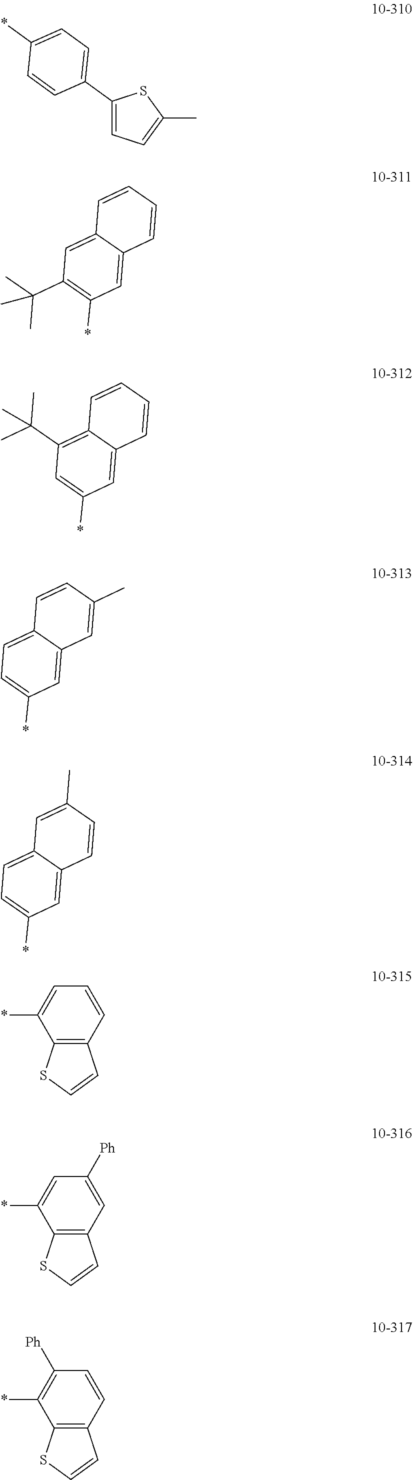 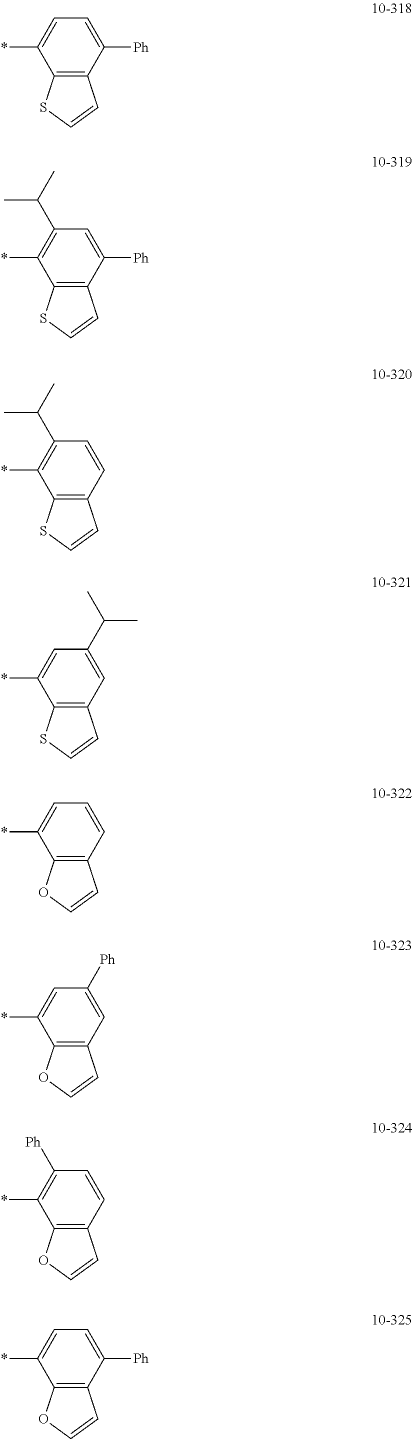 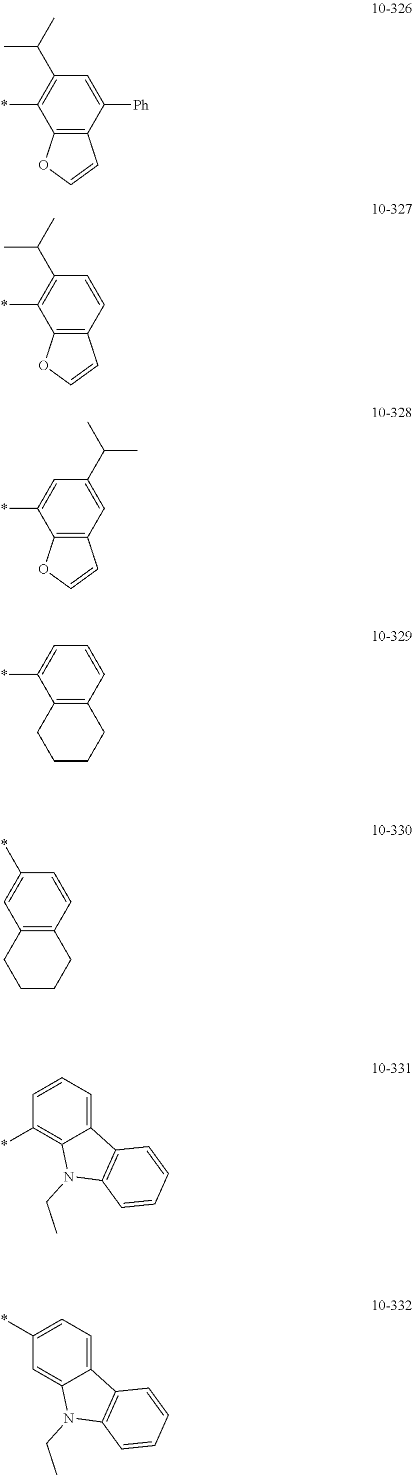 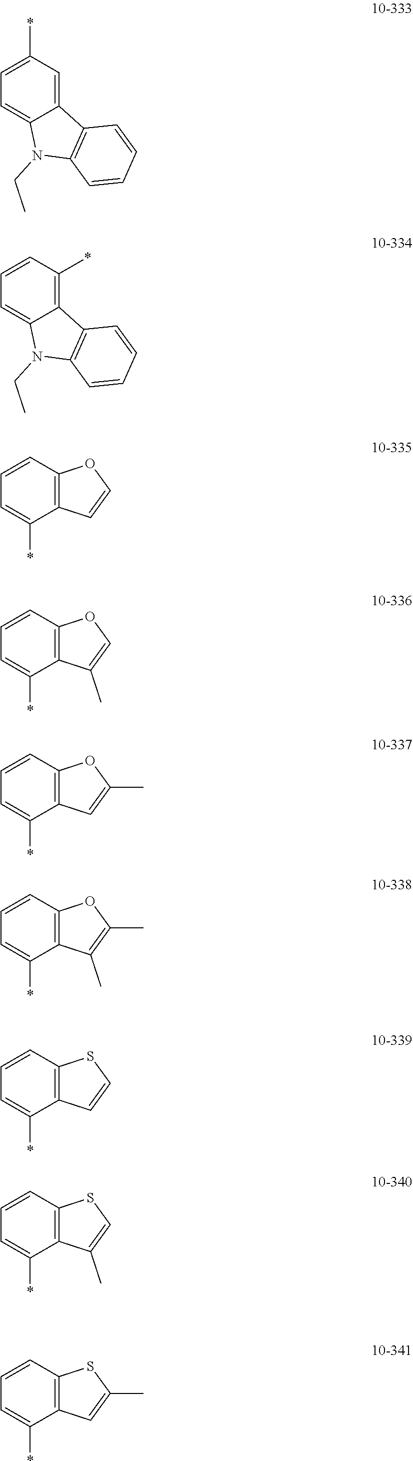 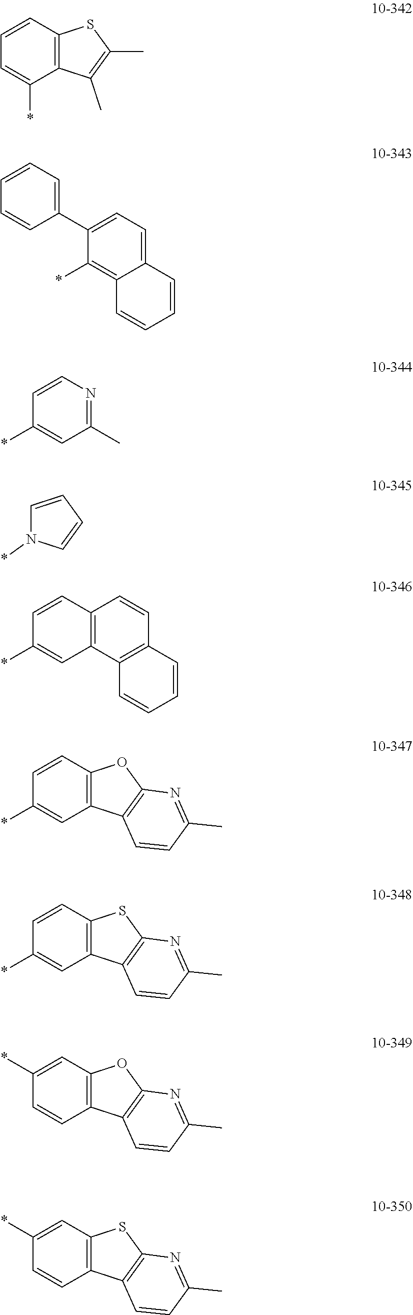
wherein * in Formulae 9-1 to 9-67, 9-201 to 9-244, 10-1 to 10-154, and 10-201 to 10-350 indicates a binding site to a neighboring atom, Ph is a phenyl group, TMS is a trimethylsilyl group, and TMG is a trimethylgermyl group.

10. The organometallic compound of claim 1, wherein *-(L₁)ₐ₁-(Ar₁)ₖ₁ in Formula 1B is a group represented by one of Formulae 3-1 to 3-18: 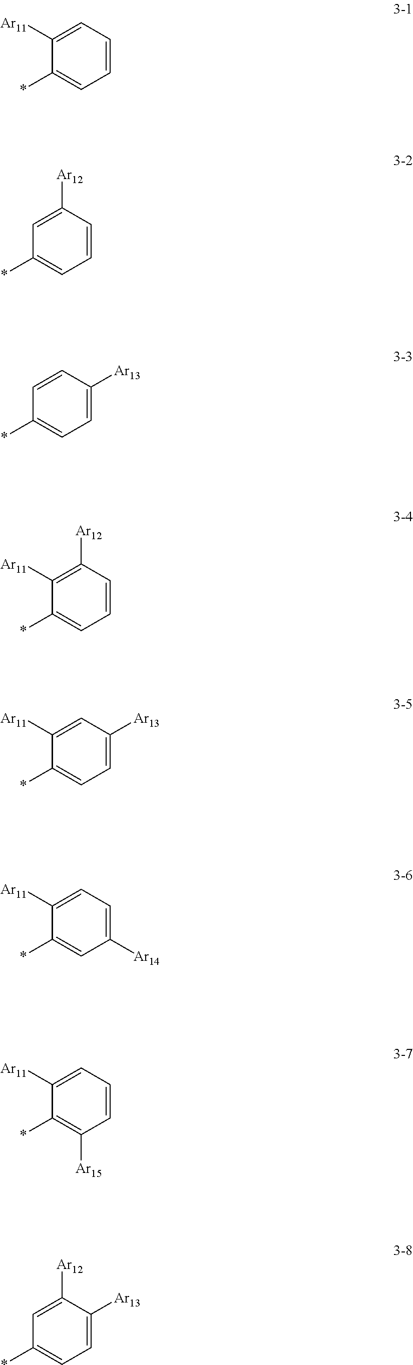 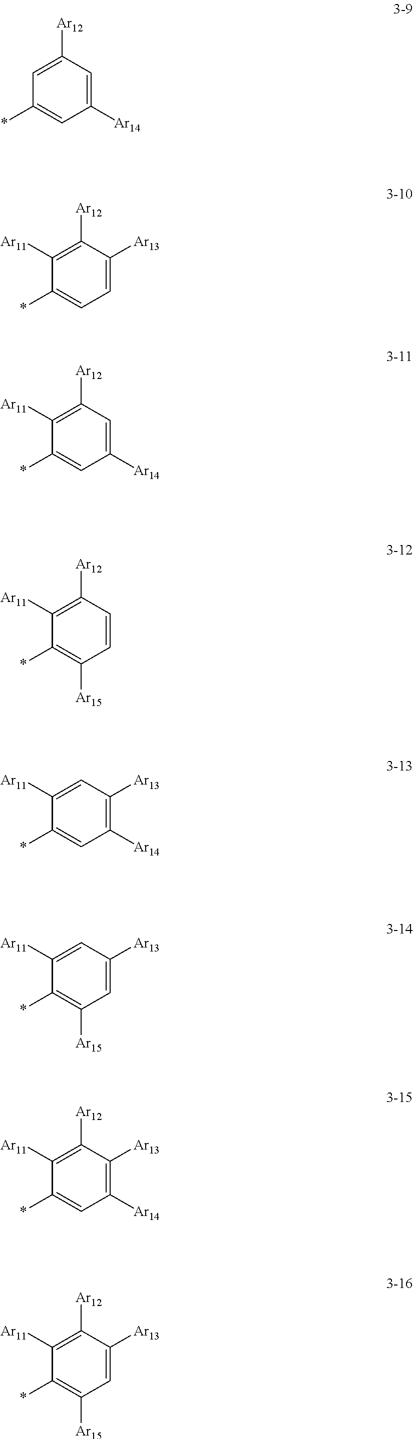 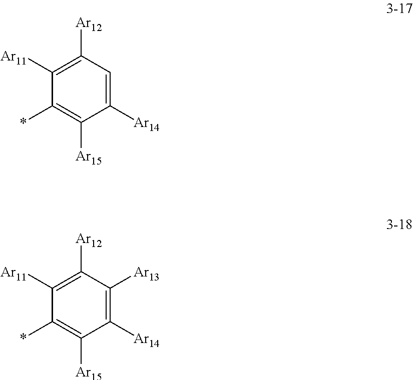
wherein, in Formulae 3-1 to 3-18,
Ar₁₁ to Ar₁₅ are each independently as described in connection with Ar₁ in claim 1, and
* indicates a binding site to a neighboring atom.

11. The organometallic compound of claim 1, wherein at least one of Ar₁, R₁₀, R₂₀, R₃₀, and R₄₀ includes —F.

12. The organometallic compound of claim 1, wherein the structure represented by: 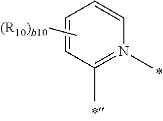
in Formula 1A is represented by one of Formulae 1-1 to 1-16: 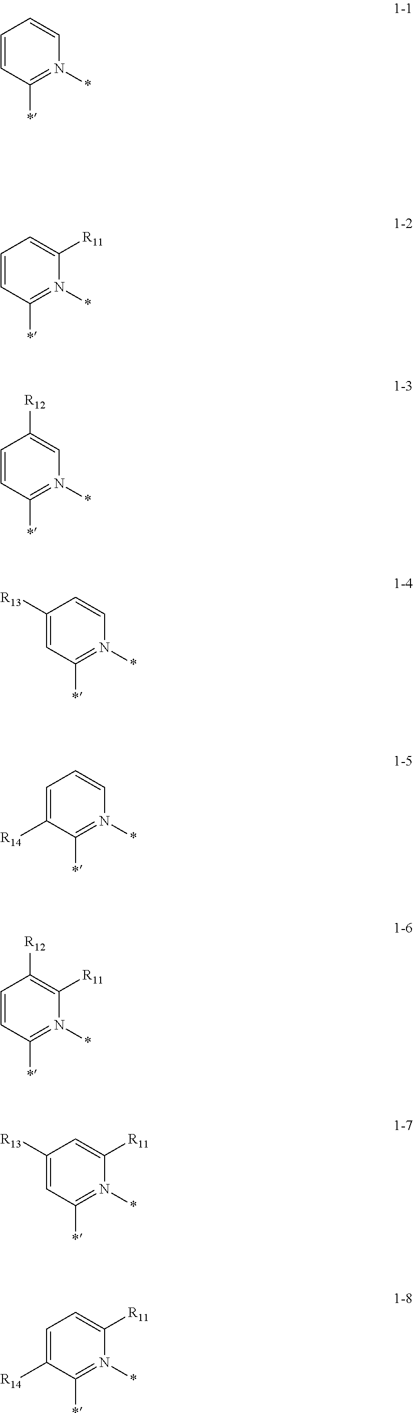 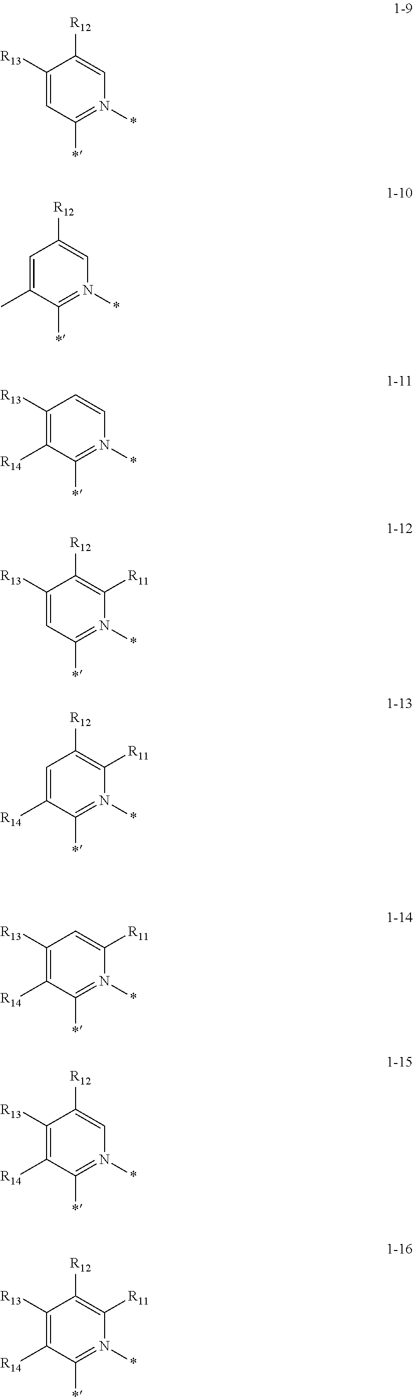
wherein, in Formulae 1-1 to 1-16,
R₁₁ to R₁₄ are each independently deuterium, —F, —Cl, —Br, —I, —CD₃, —CD₂H, —CDH₂, —CF₃, —CF₂H, —CFH₂, a methyl group, an ethyl group, an n-propyl group, an isopropyl group, an n-butyl group, an isobutyl group, a sec-butyl group, a tert-butyl group, an n-pentyl group, an isopentyl group, a 2-methylbutyl group, a sec-pentyl group, a tert-pentyl group, a neo-pentyl group, a 3-pentyl group, a 3-methyl-2-butyl group, a phenyl group, a biphenyl group, a naphthyl group, —Si(Q₁)(Q₂)(Q₃), or —Ge(Q₁)(Q₂)(Q₃),
Q₁ to Q₃ are each independently:
—CH₃, —CD₃, —CD₂H, —CDH₂, —CH₂CH₃, —CH₂CD₃, —CH₂CD₂H, —CH₂CDH₂, —CHDCH₃, —CHDCD₂H, —CHDCDH₂, —CHDCD₃, —CD₂CD₃, —CD₂CD₂H, or —CD₂CDH₂;
an n-propyl group, an isopropyl group, an n-butyl group, an isobutyl group, a sec-butyl group, a tert-butyl group, an n-pentyl group, an isopentyl group, a sec-pentyl group, a tert-pentyl group, a phenyl group, or a naphthyl group; or
an n-propyl group, an isopropyl group, an n-butyl group, an isobutyl group, a sec-butyl group, a tert-butyl group, an n-pentyl group, an isopentyl group, a sec-pentyl group, a tert-pentyl group, a phenyl group, or a naphthyl group, each substituted with deuterium, a C₁-C₁₀ alkyl group, a phenyl group, or a combination thereof, and
* and *′ each indicate a binding site to a neighboring atom.

13. The organometallic compound of claim 1, wherein the structure represented by: 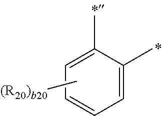
in Formula 1 is represented by one of Formulae 2-1 to 2-16: 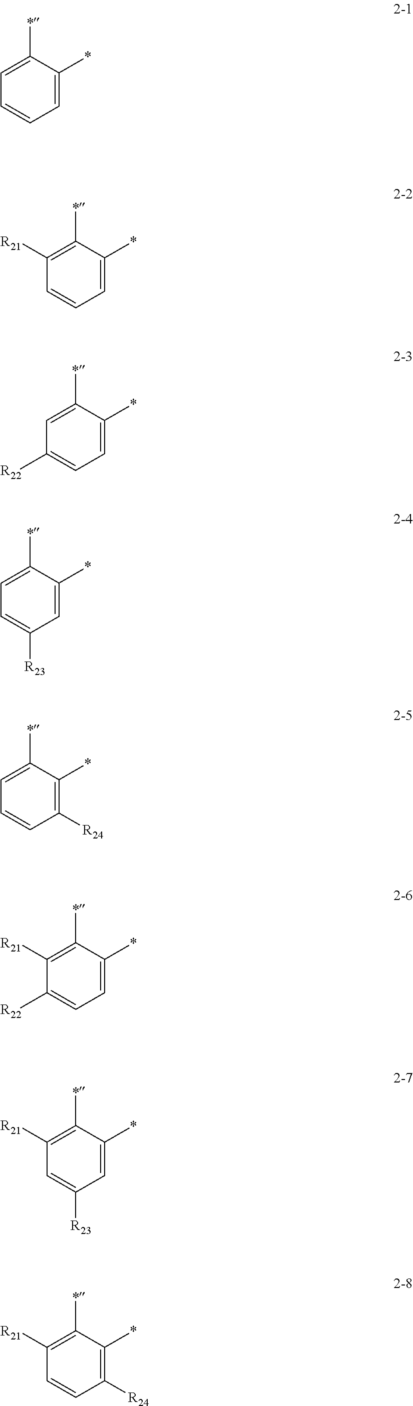 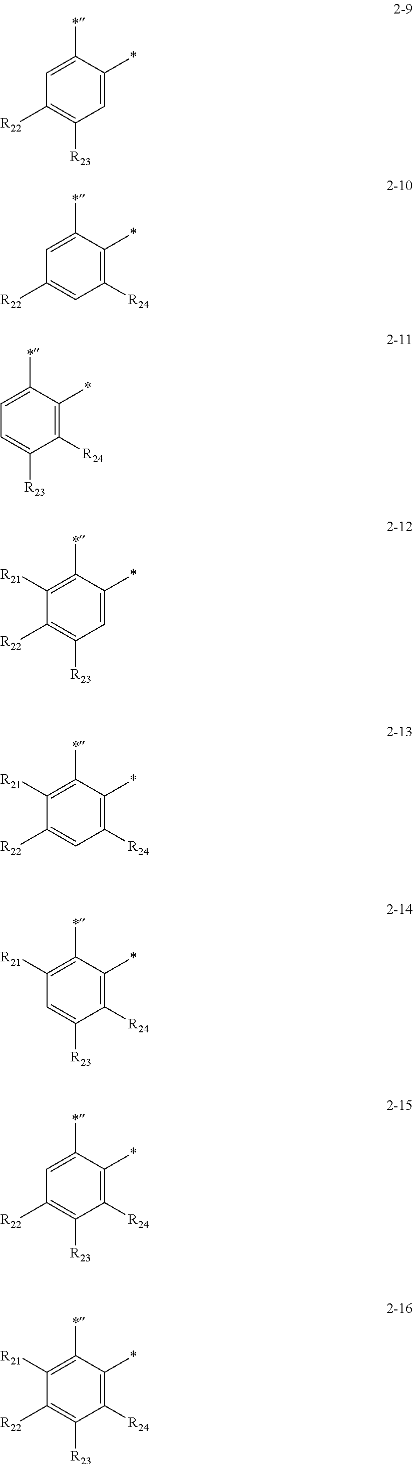
wherein, in Formulae 2-1 to 2-16,
R₂₁ to R₂₄ are each independently deuterium, —F, —Cl, —Br, —I, —CD₃, —CD₂H, —CDH₂, —CF₃, —CF₂H, —CFH₂, a methyl group, an ethyl group, an n-propyl group, an isopropyl group, an n-butyl group, an isobutyl group, a sec-butyl group, a tert-butyl group, an n-pentyl group, an isopentyl group, a 2-methylbutyl group, a sec-pentyl group, a tert-pentyl group, a neo-pentyl group, a 3-pentyl group, a 3-methyl-2-butyl group, a phenyl group, a biphenyl group, a naphthyl group, —Si(Q₁)(Q₂)(Q₃), or —Ge(Q₁)(Q₂)(Q₃),
Q₁ to Q₃ are each independently:
—CH₃, —CD₃, —CD₂H, —CDH₂, —CH₂CH₃, —CH₂CD₃, —CH₂CD₂H, —CH₂CDH₂, —CHDCH₃, —CHDCD₂H, —CHDCDH₂, —CHDCD₃, —CD₂CD₃, —CD₂CD₂H, or —CD₂CDH₂;
an n-propyl group, an isopropyl group, an n-butyl group, an isobutyl group, a sec-butyl group, a tert-butyl group, an n-pentyl group, an isopentyl group, a sec-pentyl group, a tert-pentyl group, a phenyl group, or a naphthyl group; or
an n-propyl group, an isopropyl group, an n-butyl group, an isobutyl group, a sec-butyl group, a tert-butyl group, an n-pentyl group, an isopentyl group, a sec-pentyl group, a tert-pentyl group, a phenyl group, or a naphthyl group, each substituted with deuterium, a C₁-C₁₀ alkyl group, a phenyl group, or a combination thereof, and
* and *″ are each a binding site to a neighboring atom.

14. The organometallic compound of claim 1, wherein Ln₁ is a ligand represented by one of Formulae A1 to A285, and
Ln₂ is a ligand represented by one of Formulae B1 to B420:

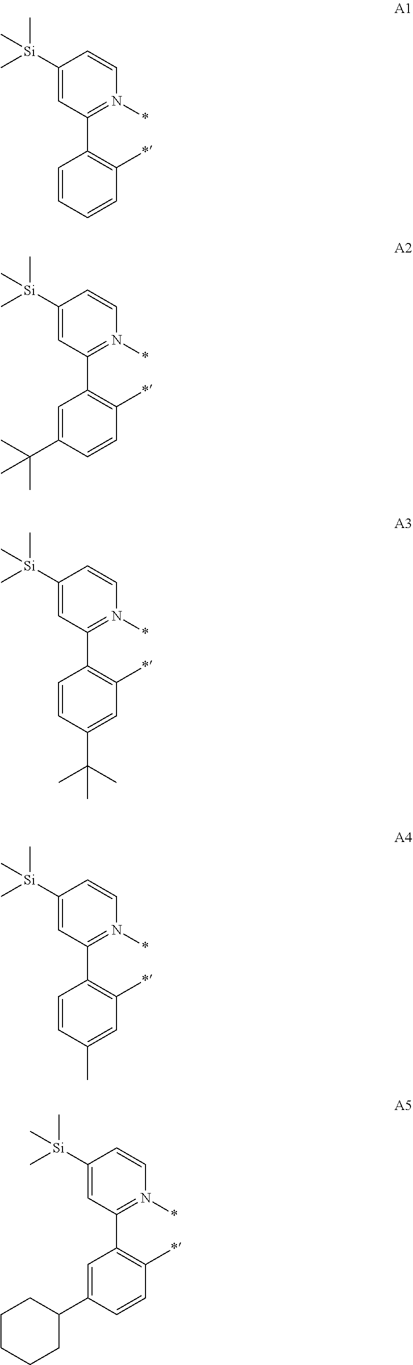

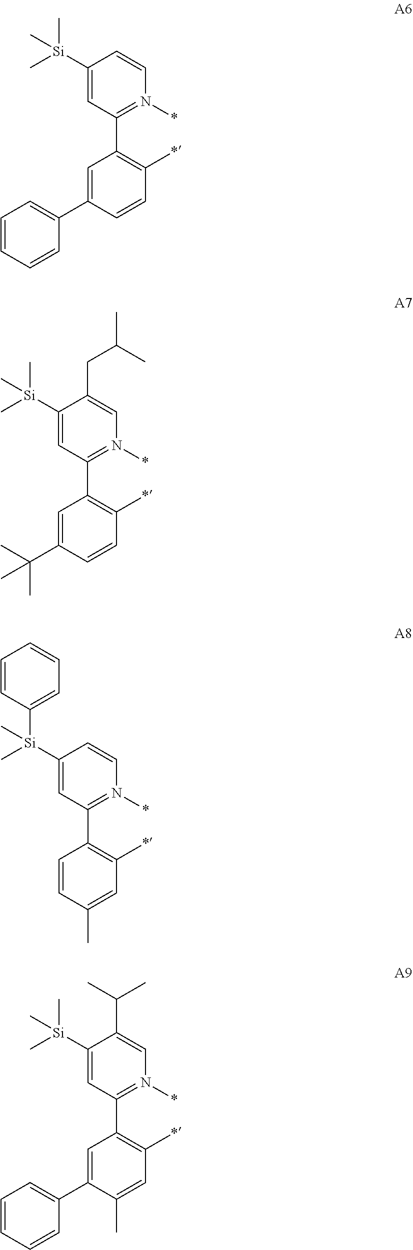

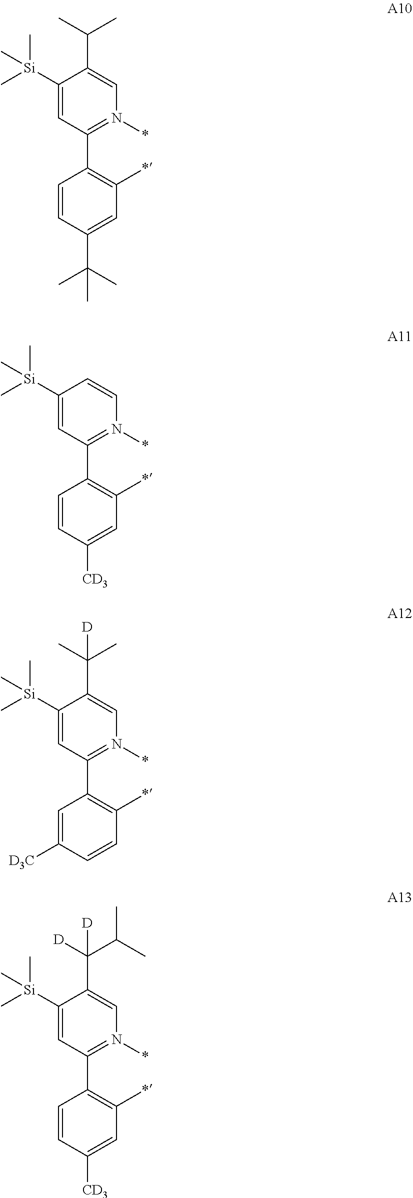

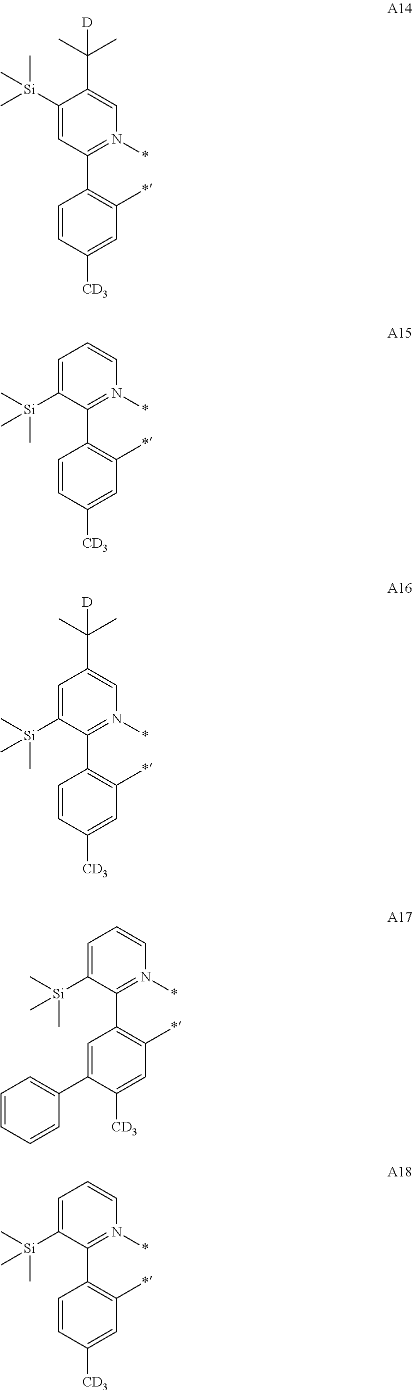

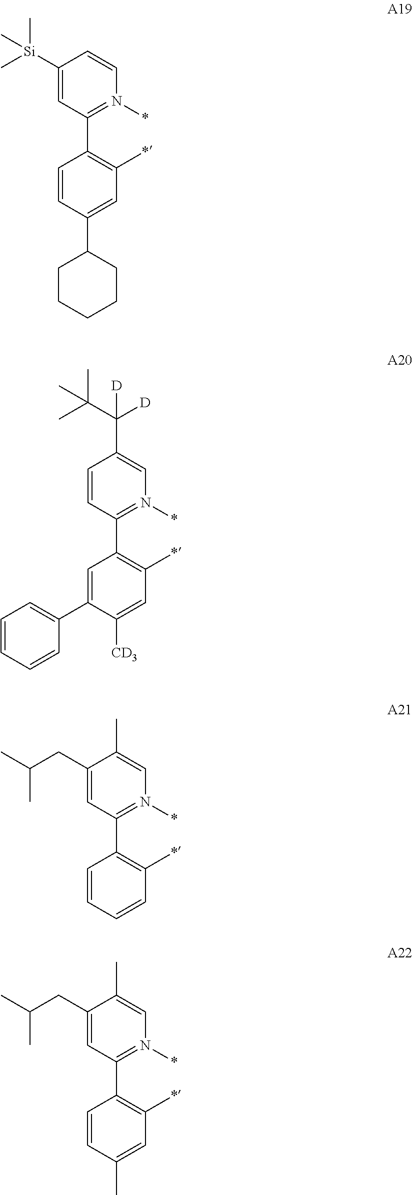

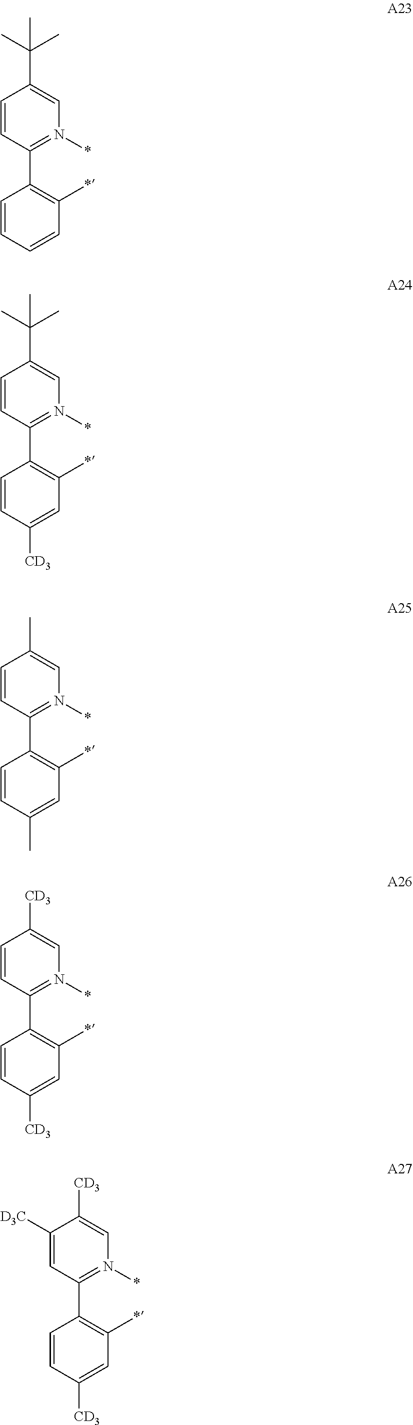

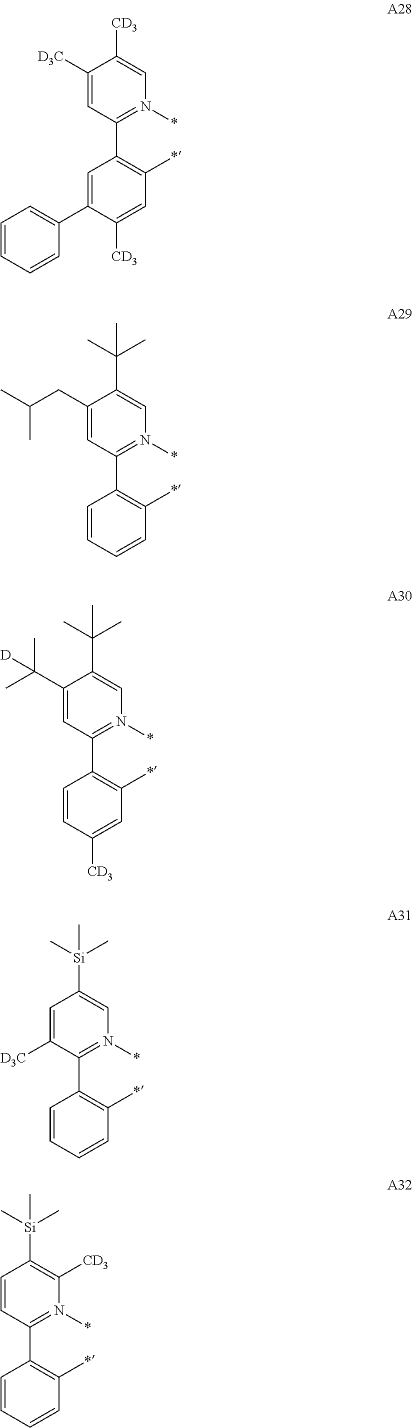

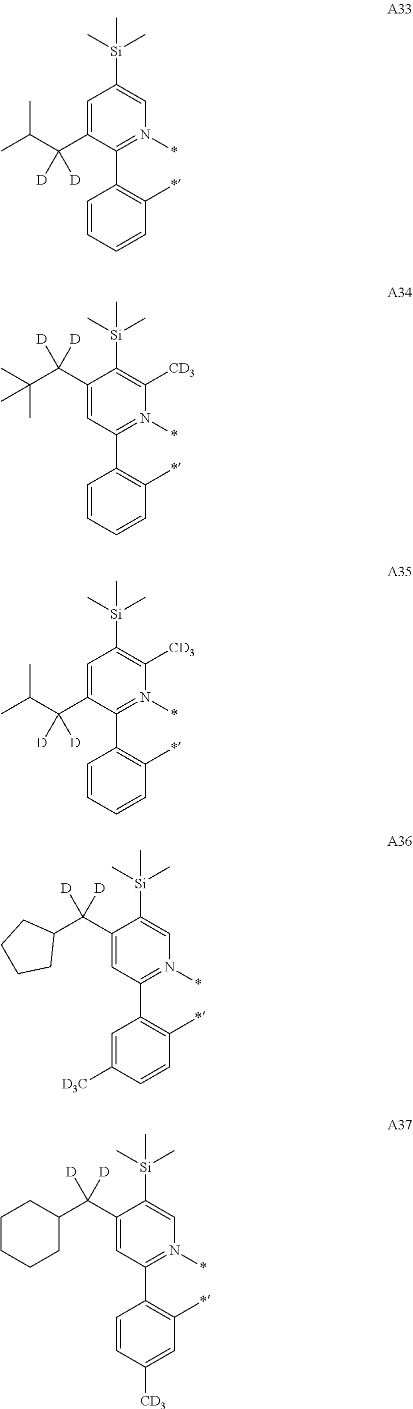

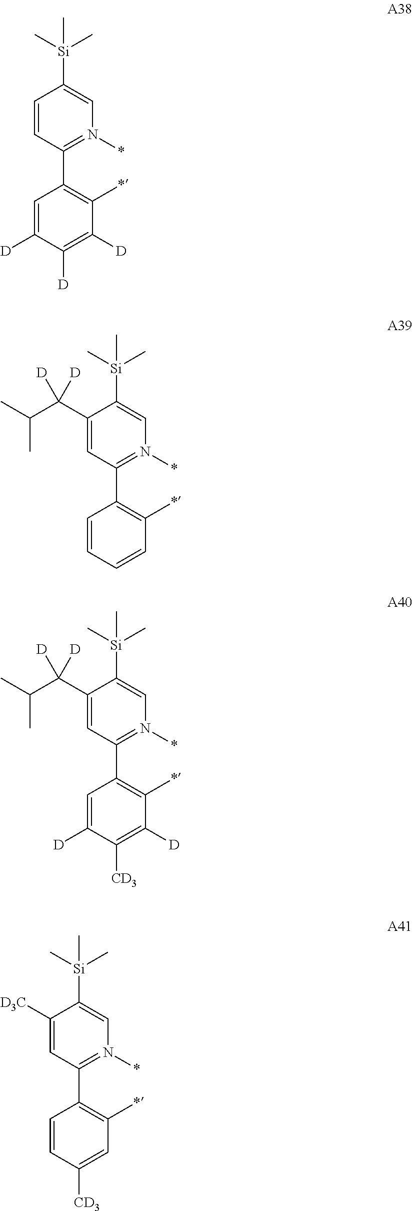

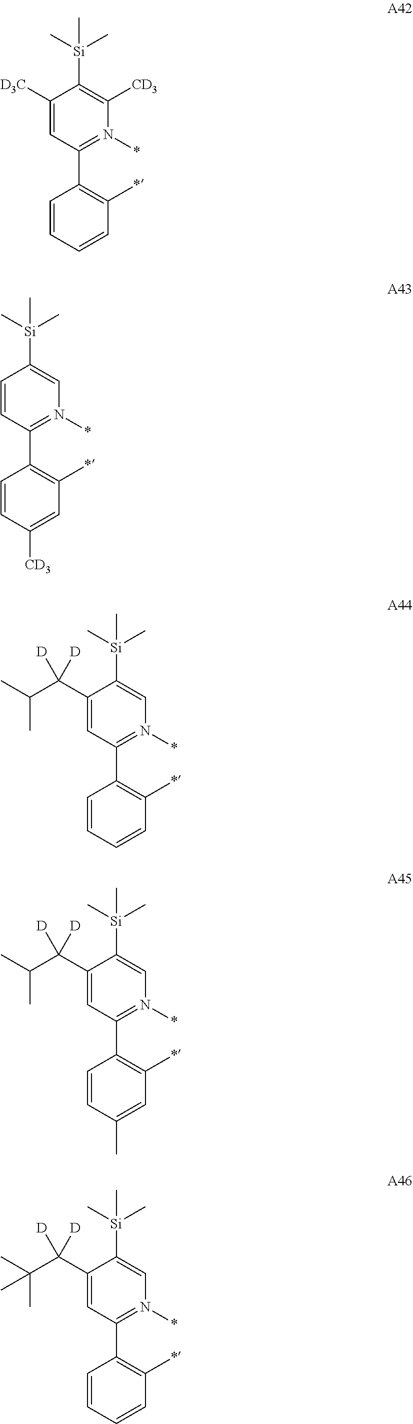

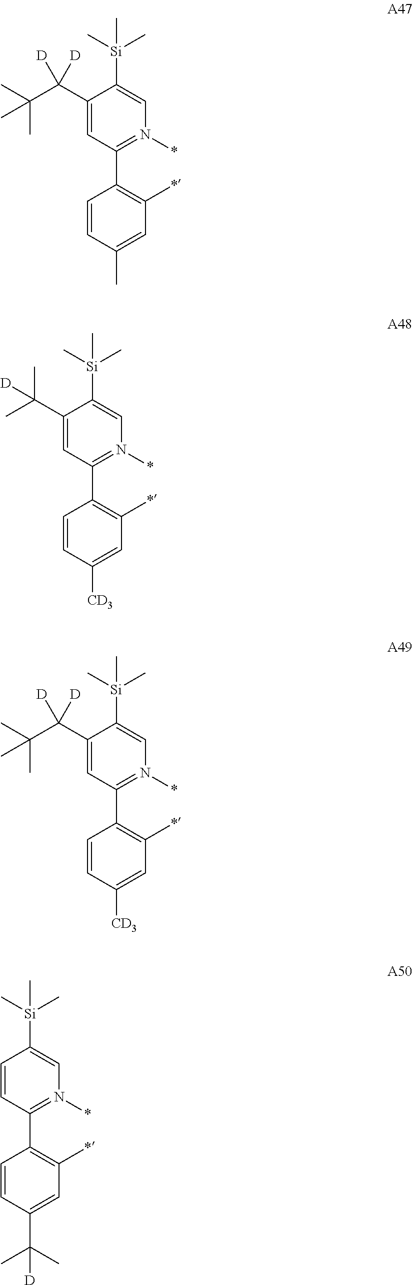

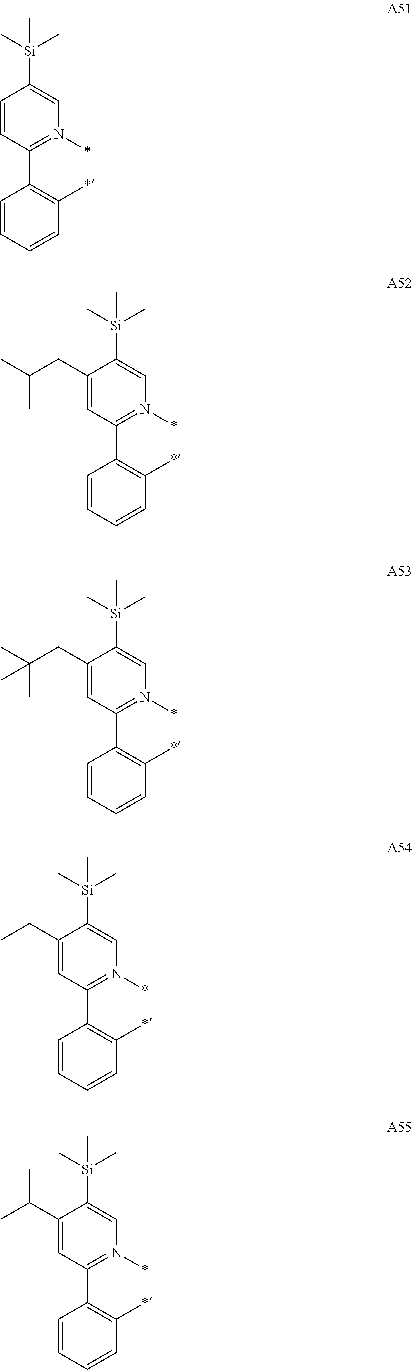

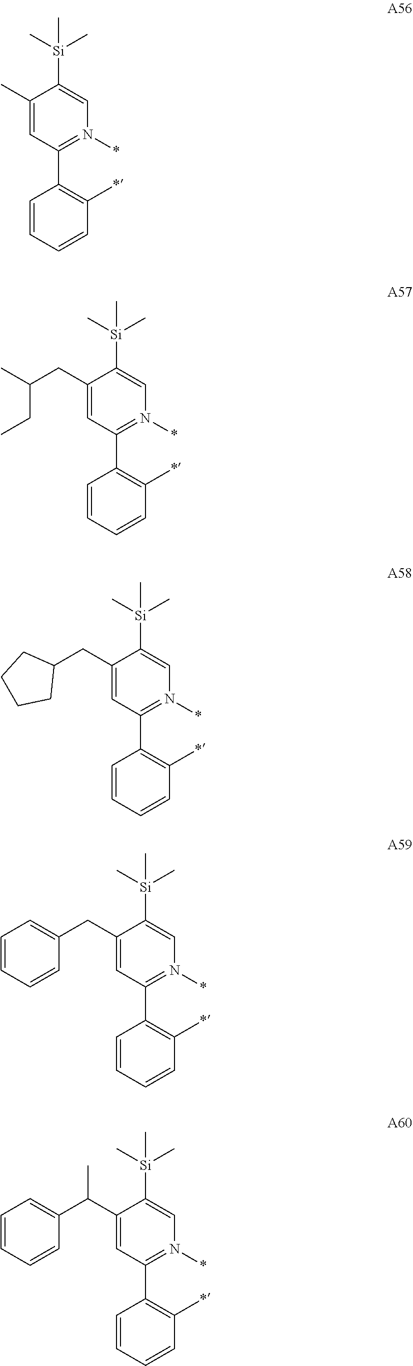

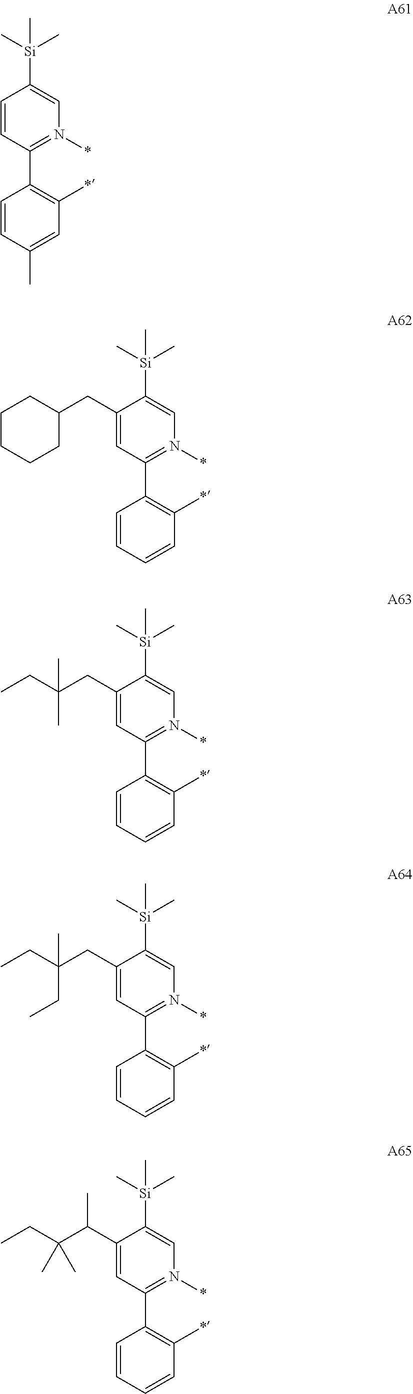

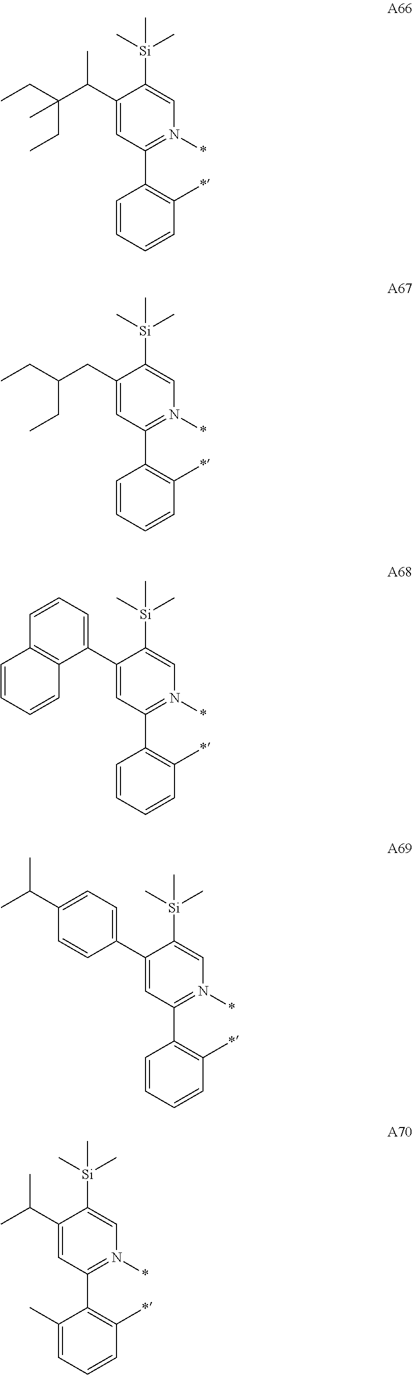

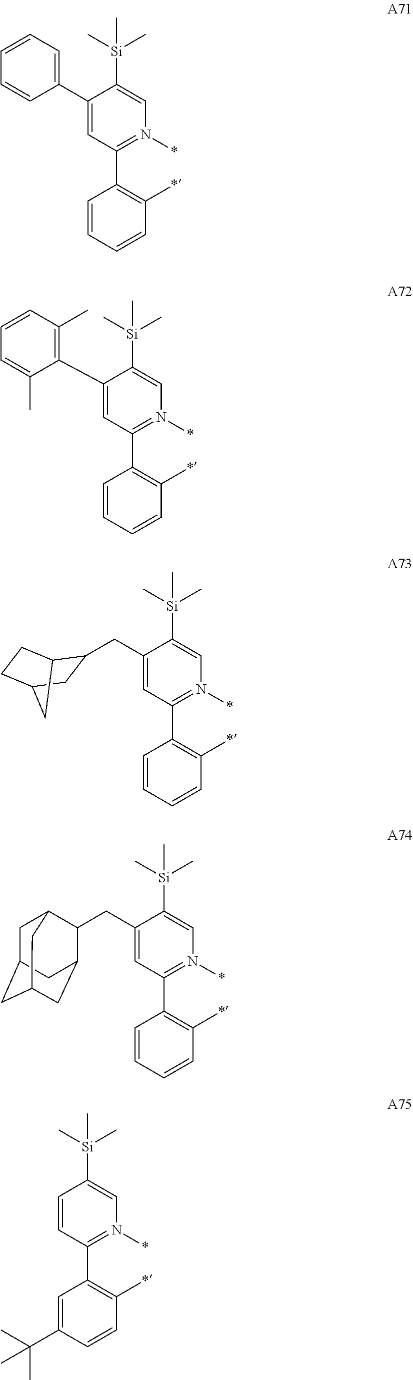

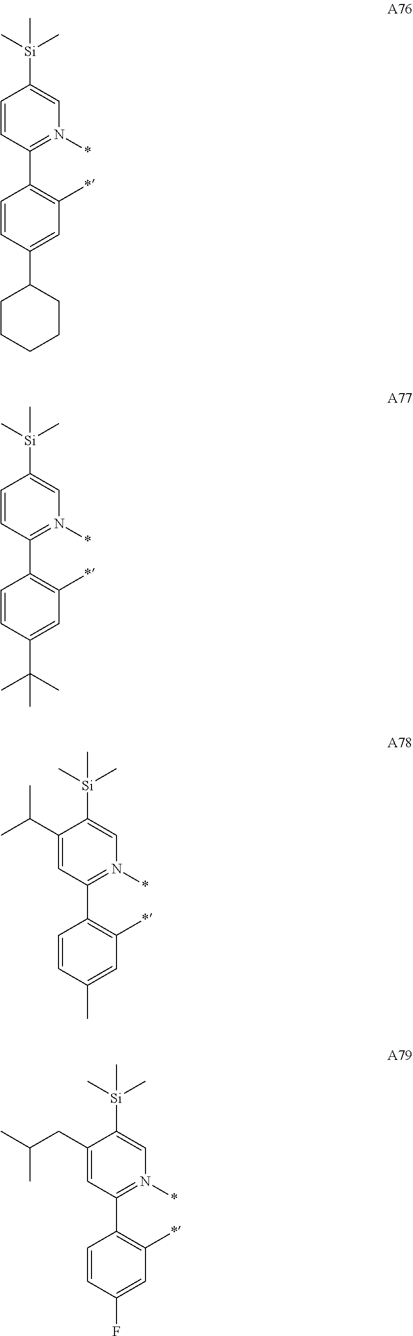

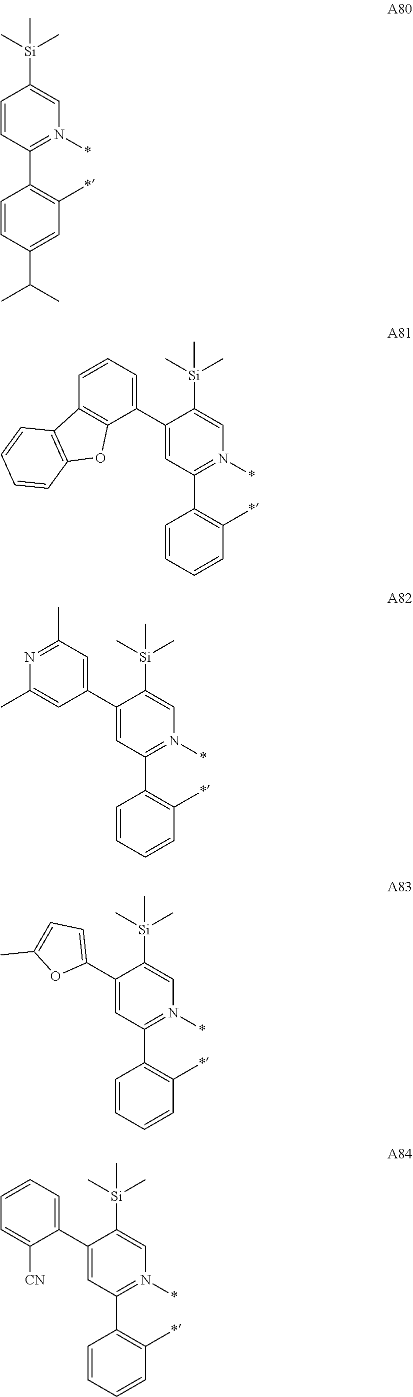

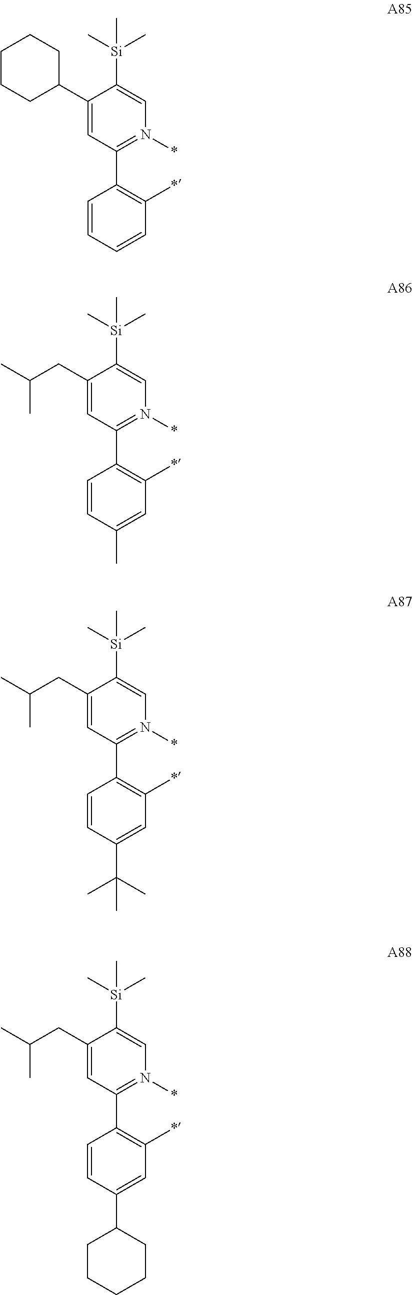

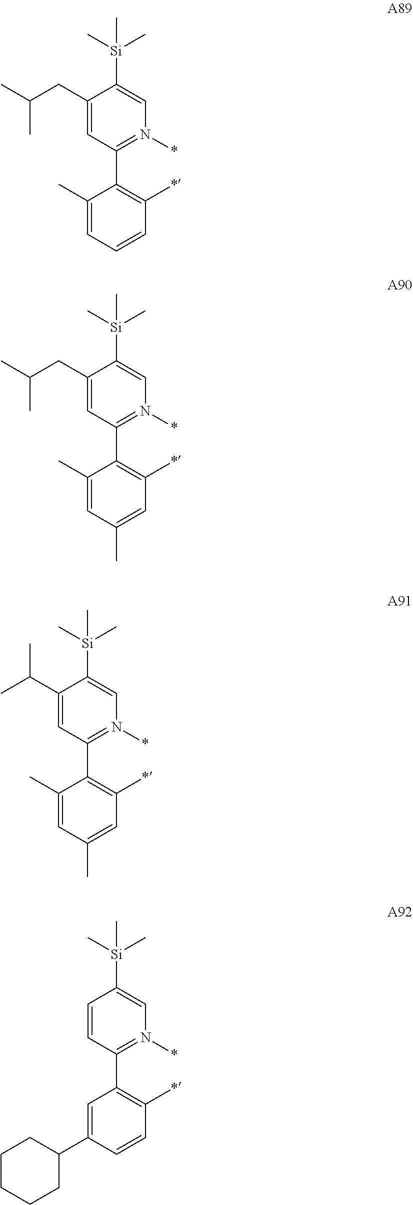

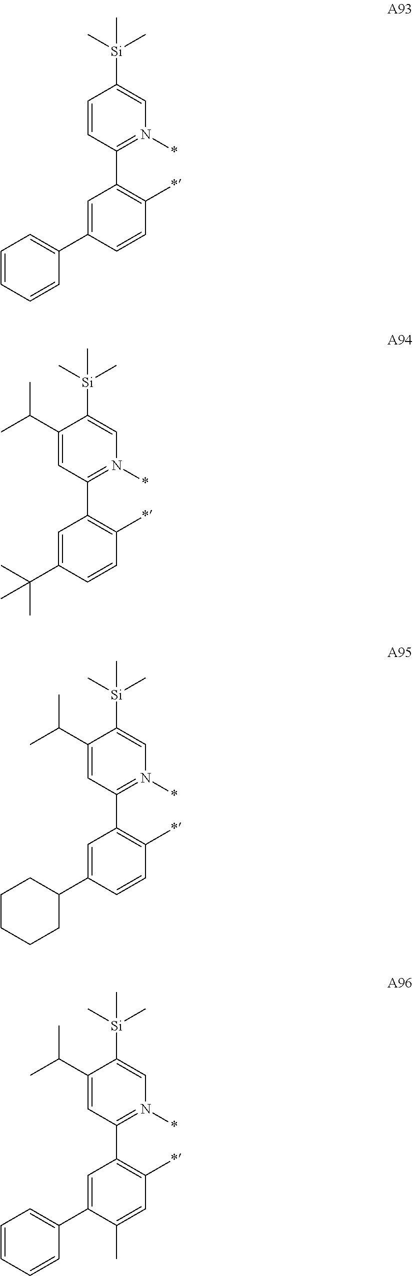

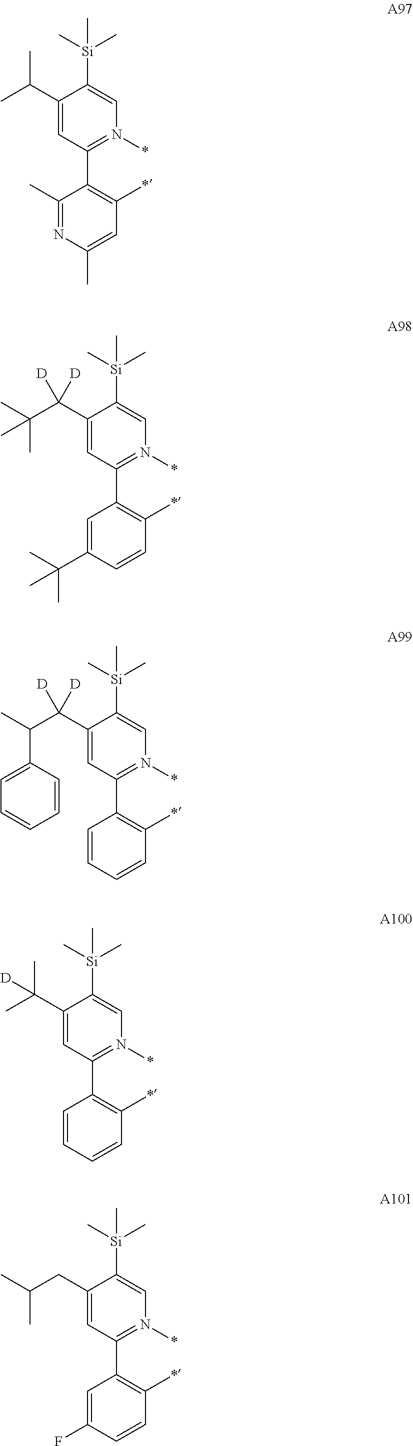

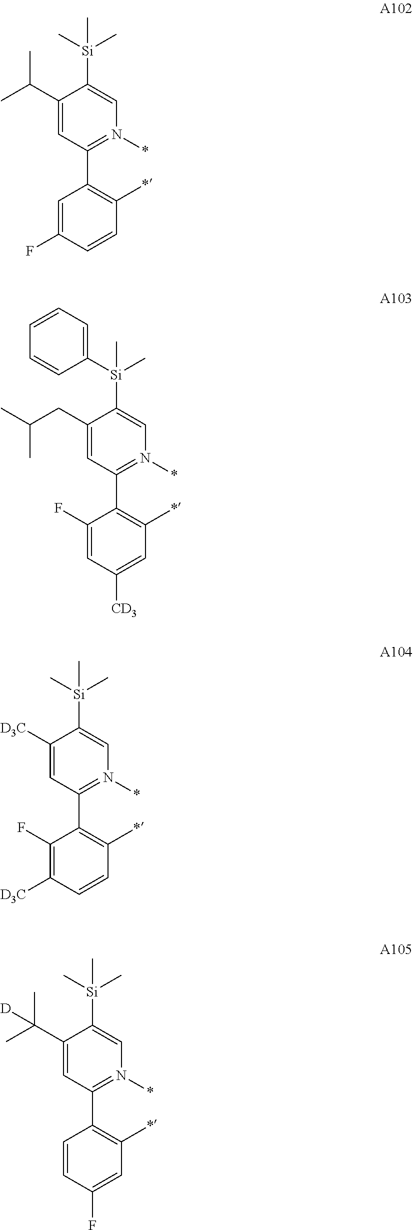

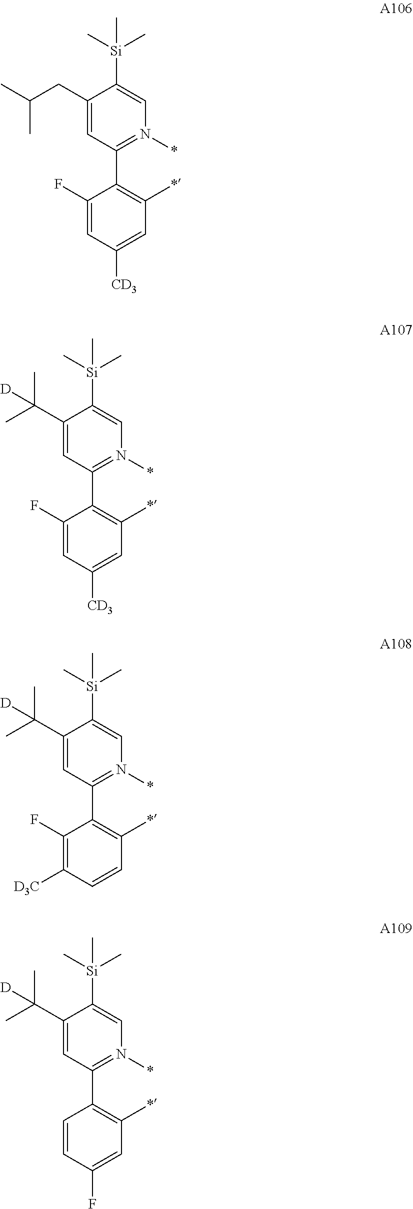

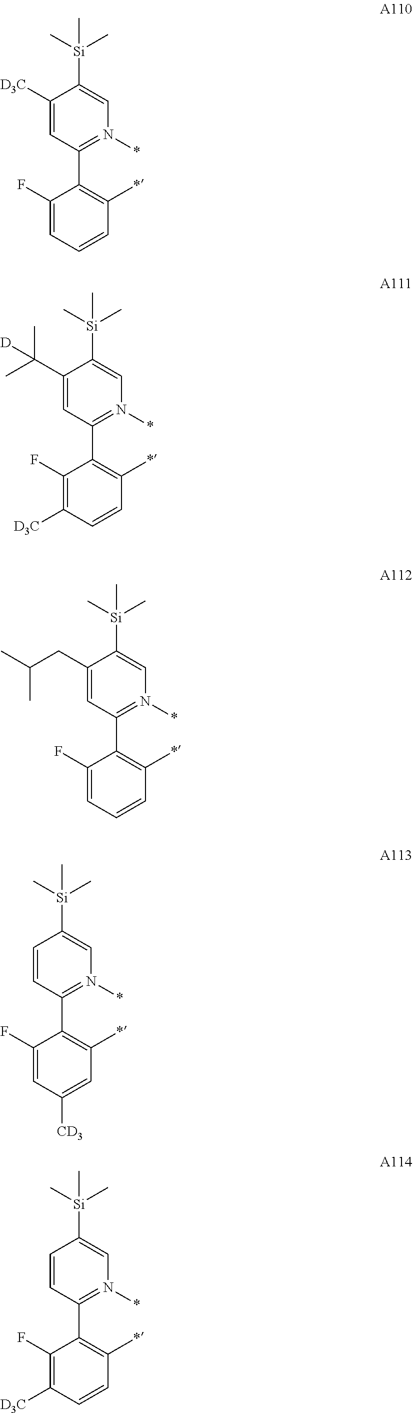

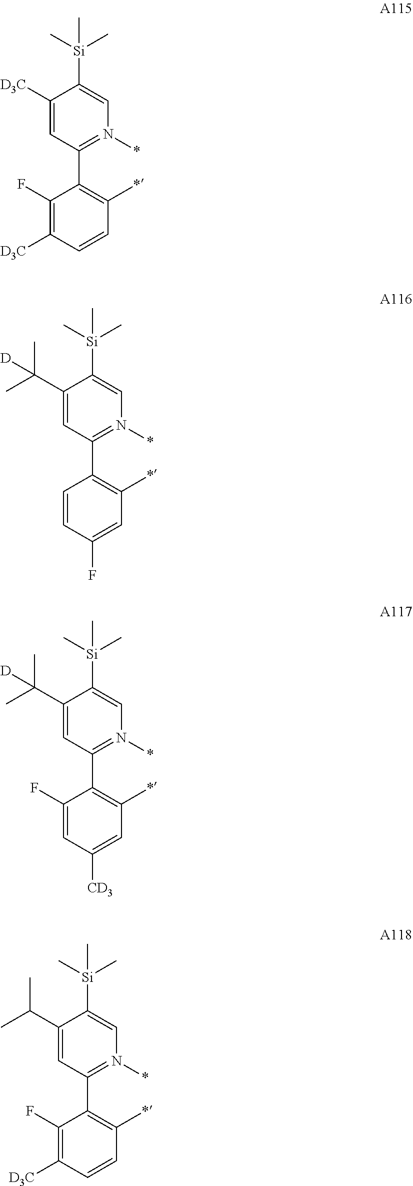

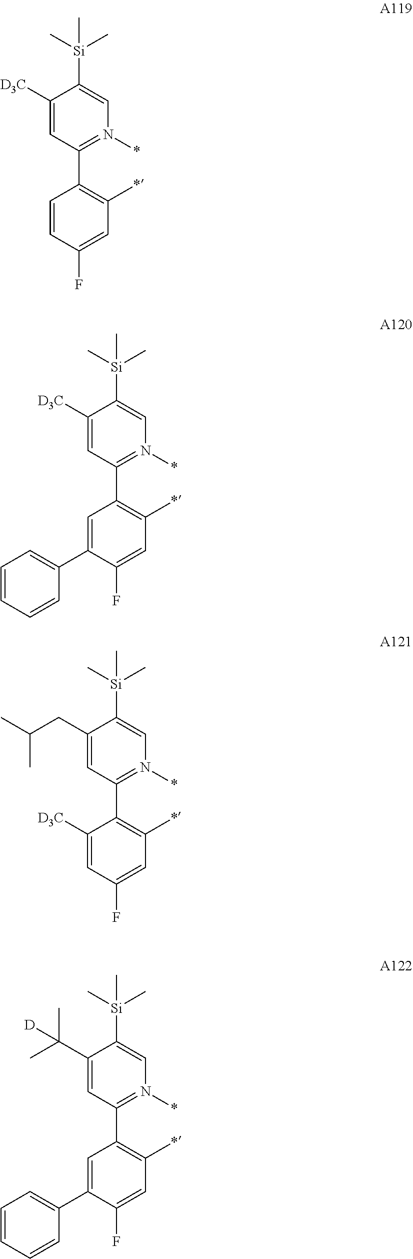

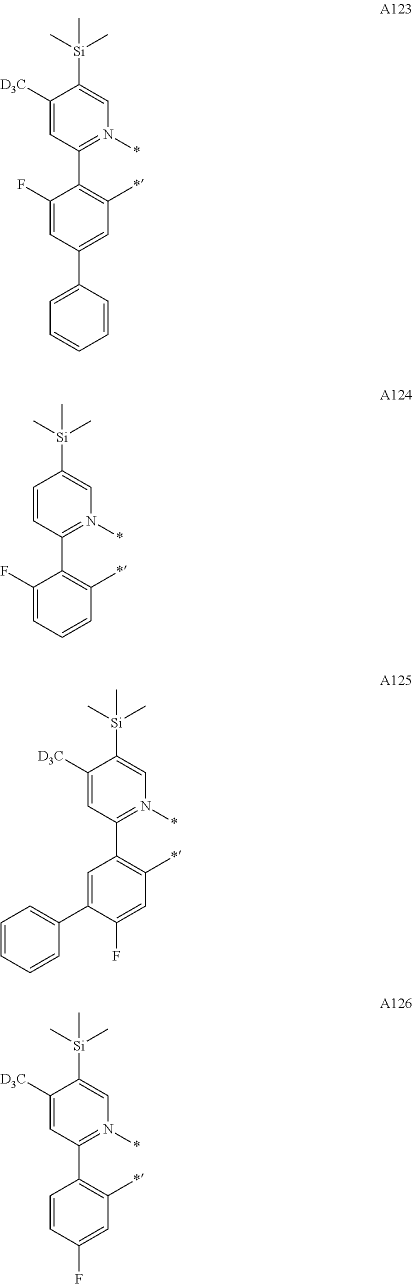

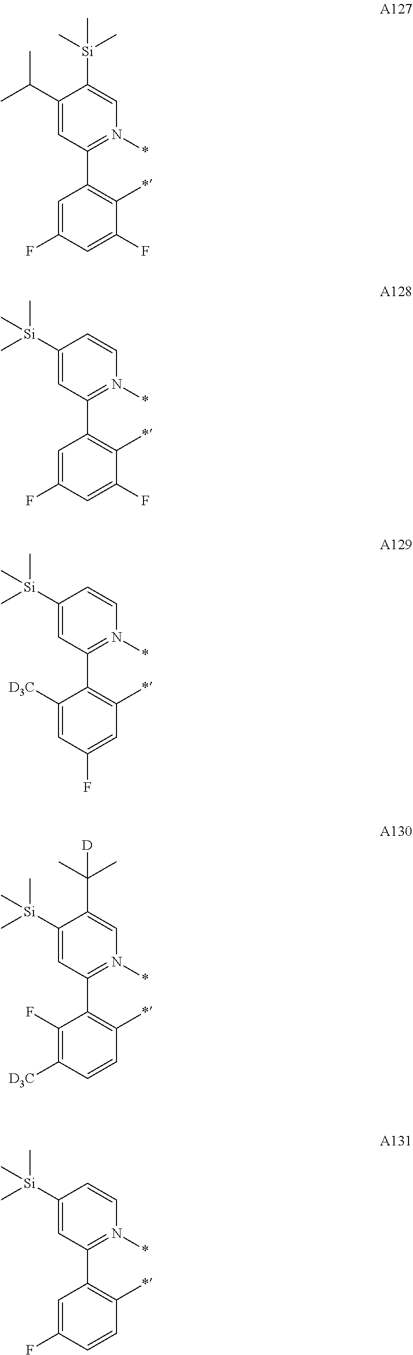

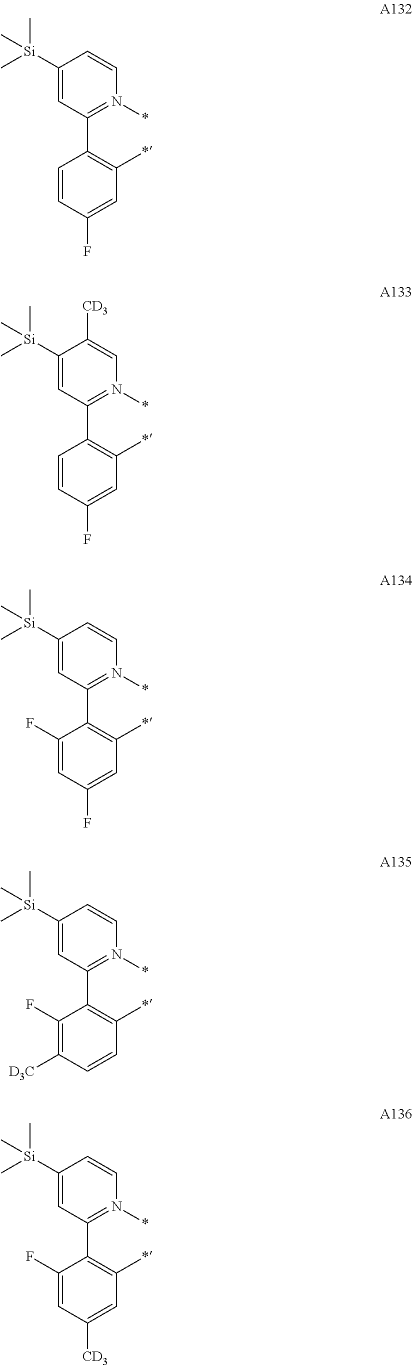

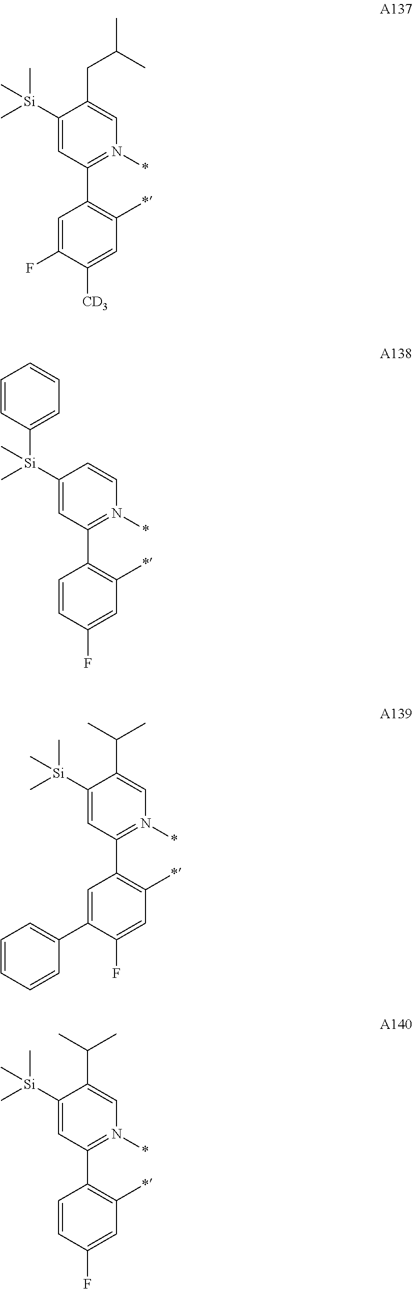

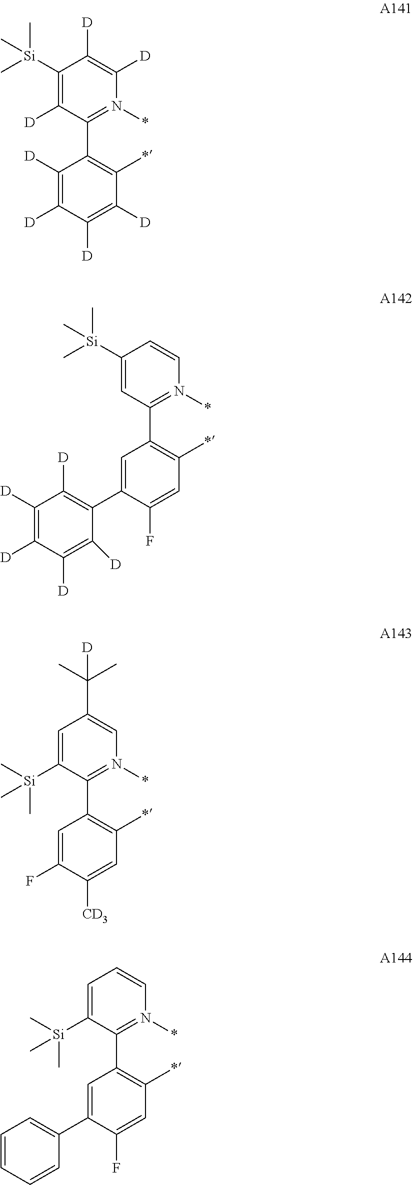

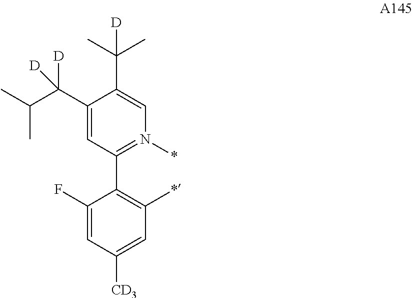

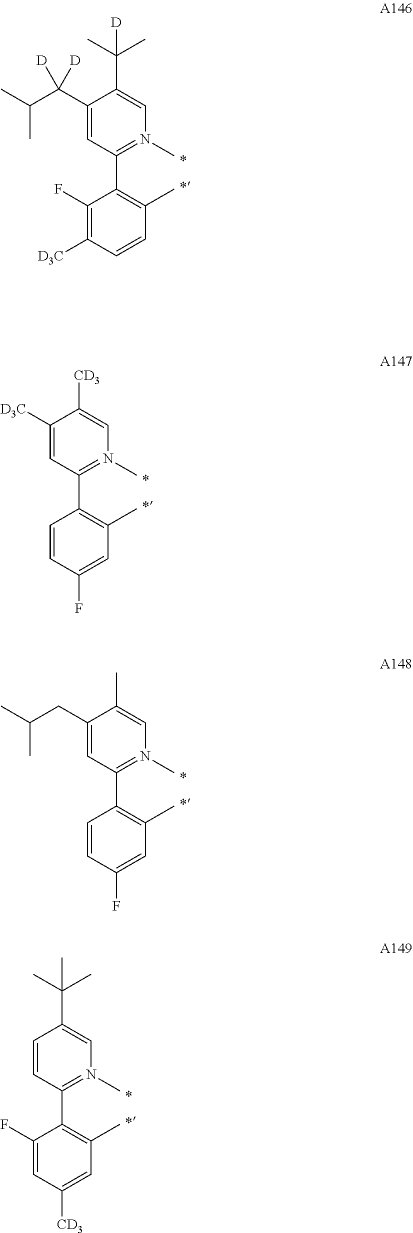

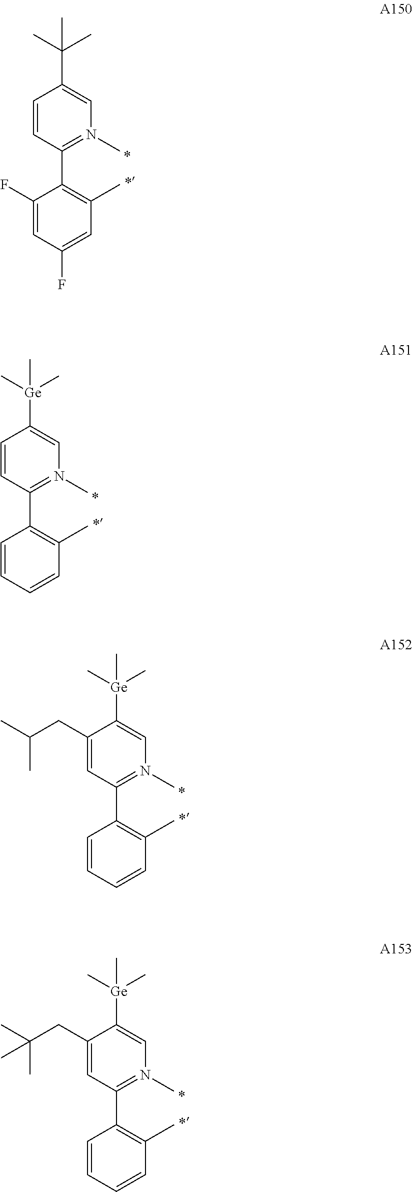

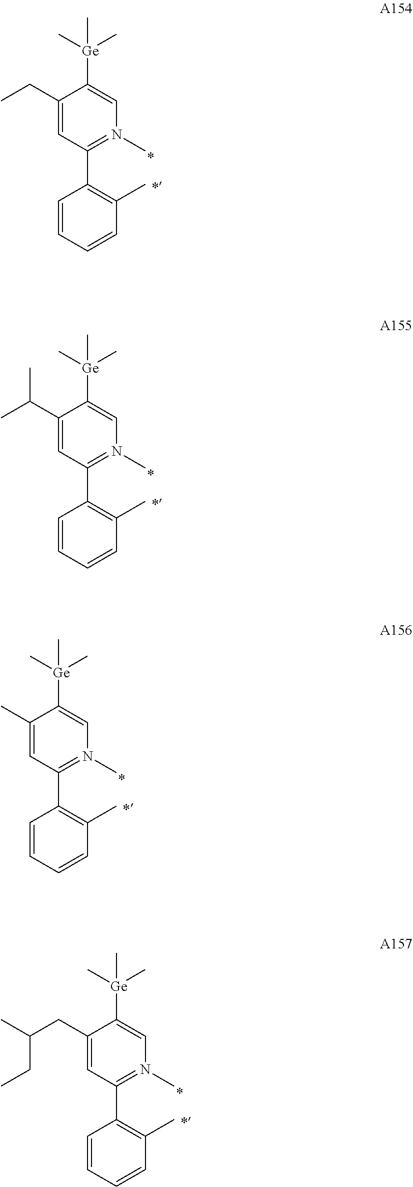

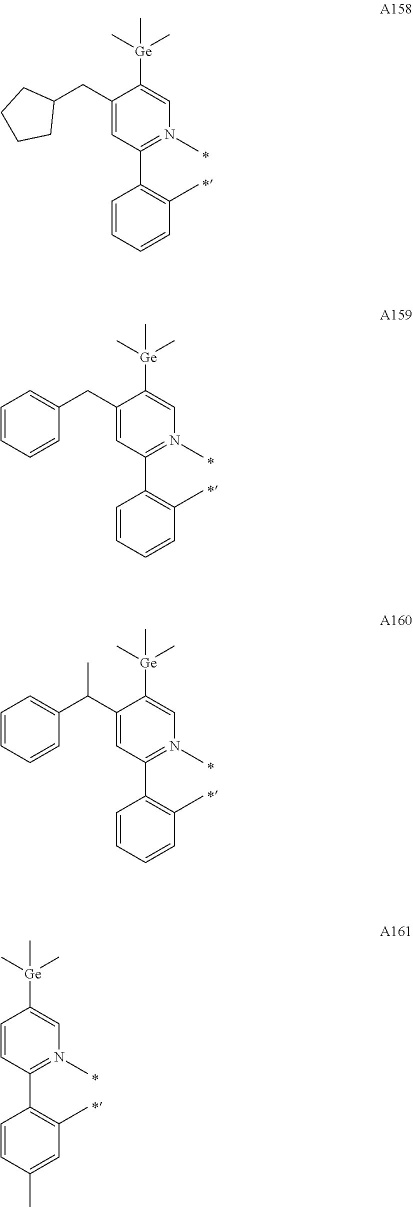

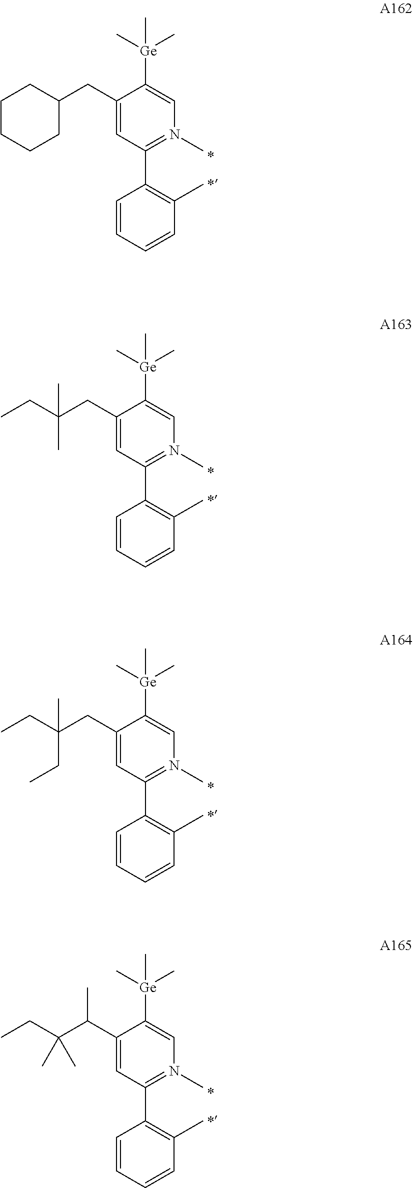

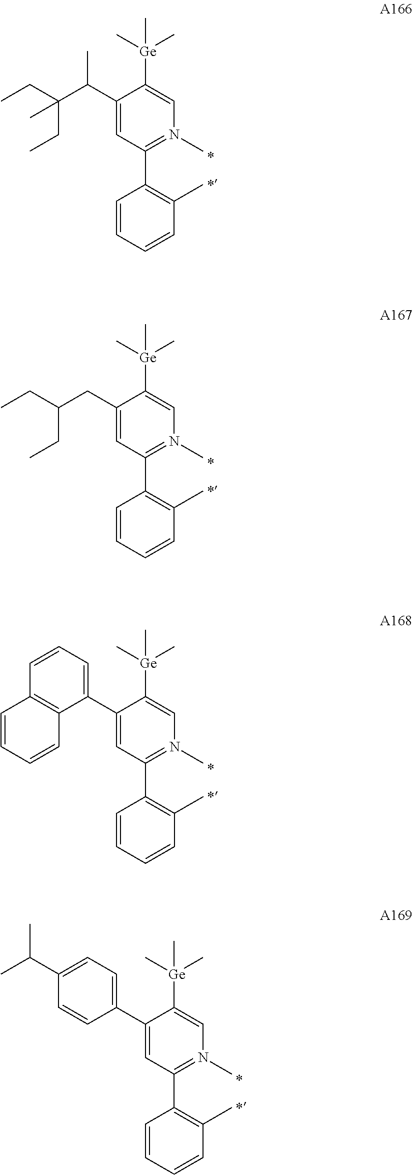

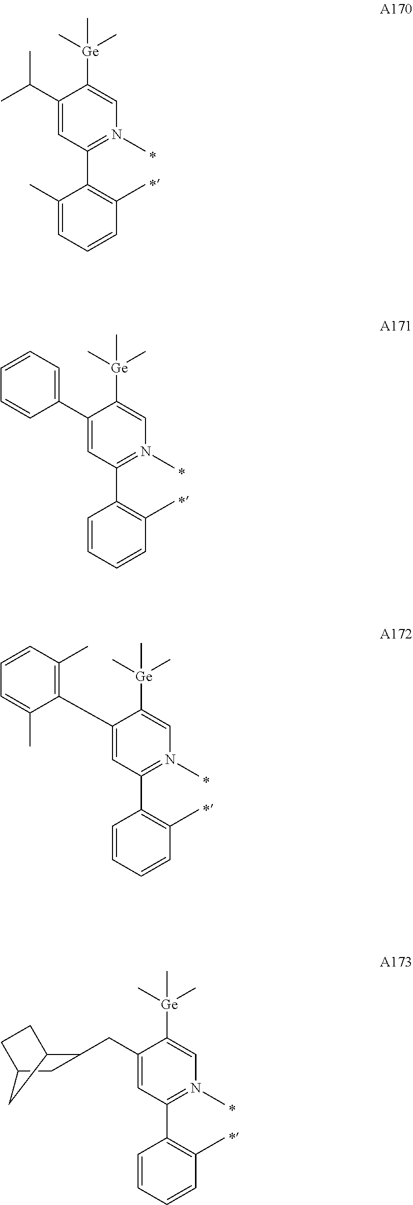

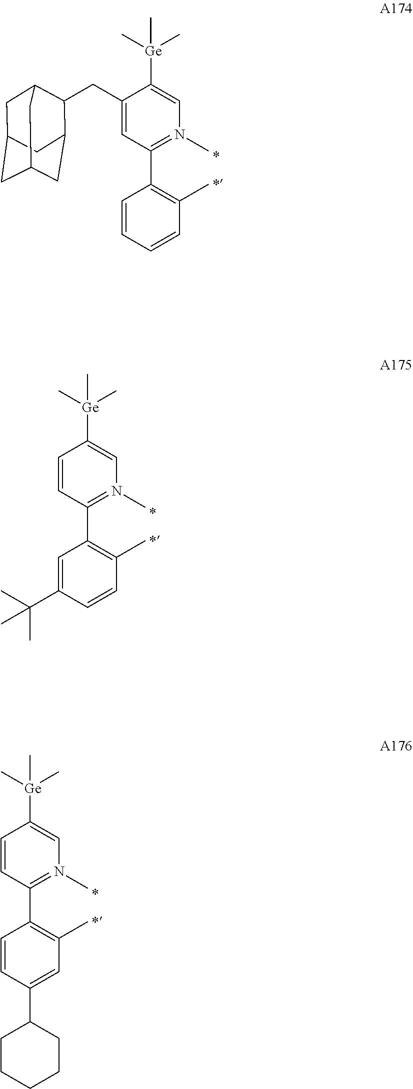

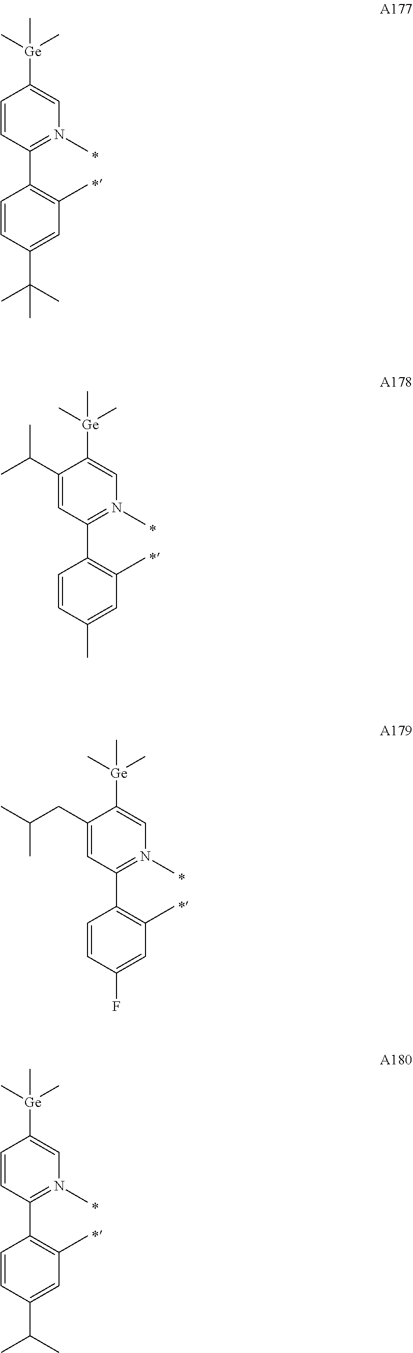

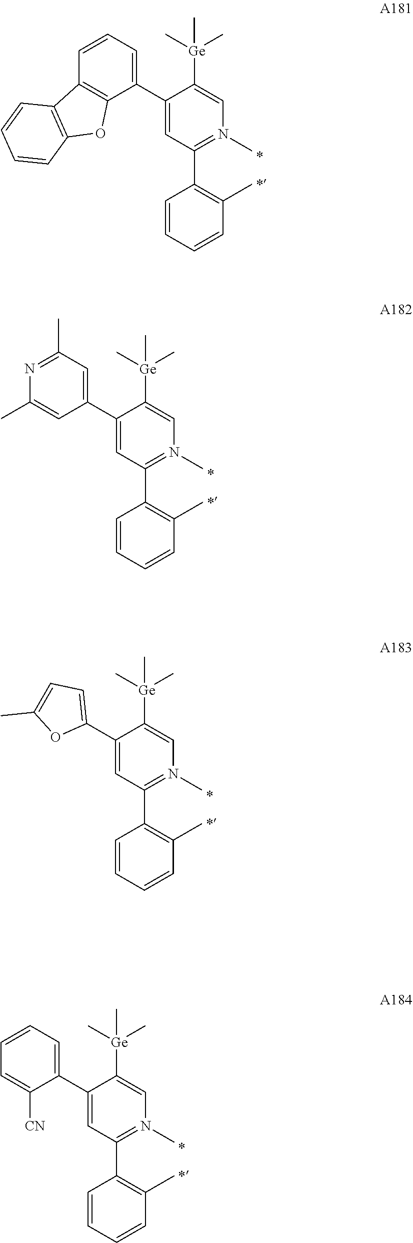

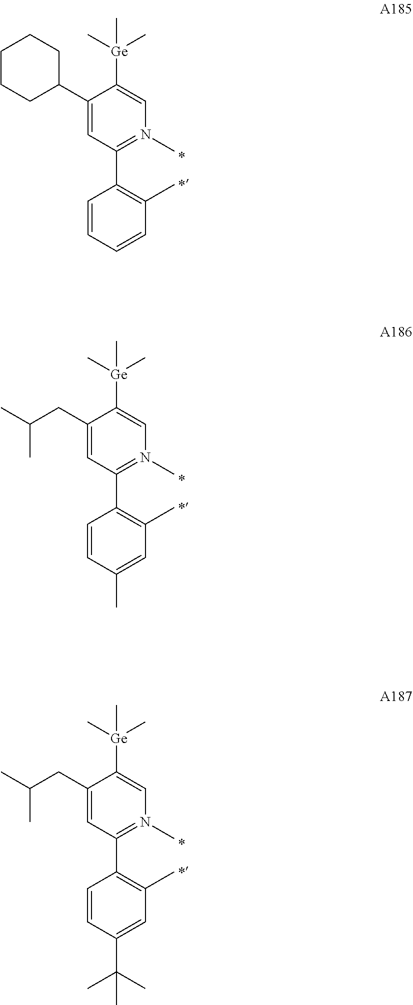

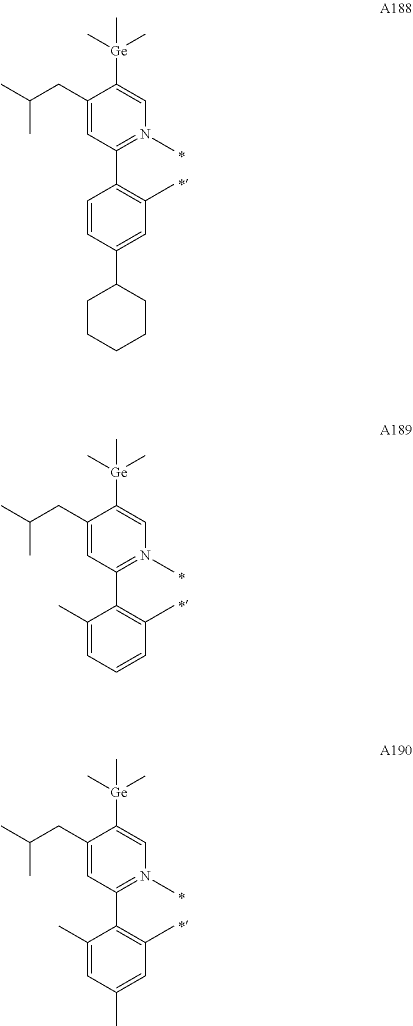

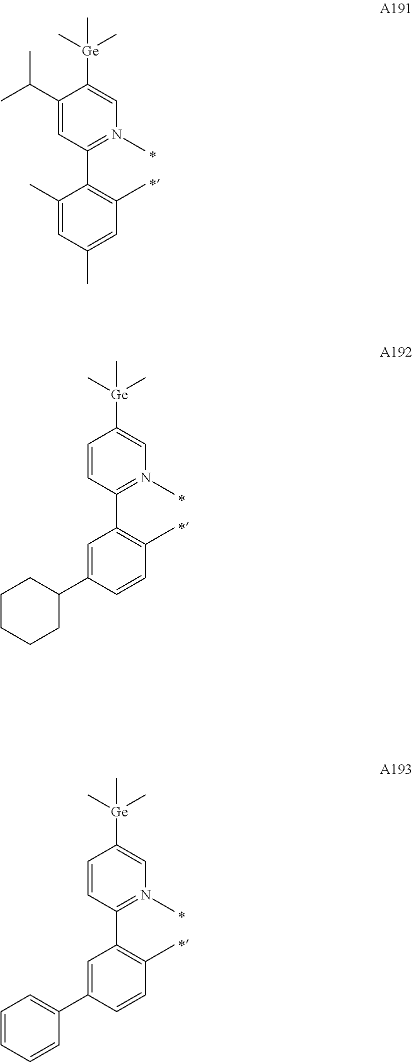

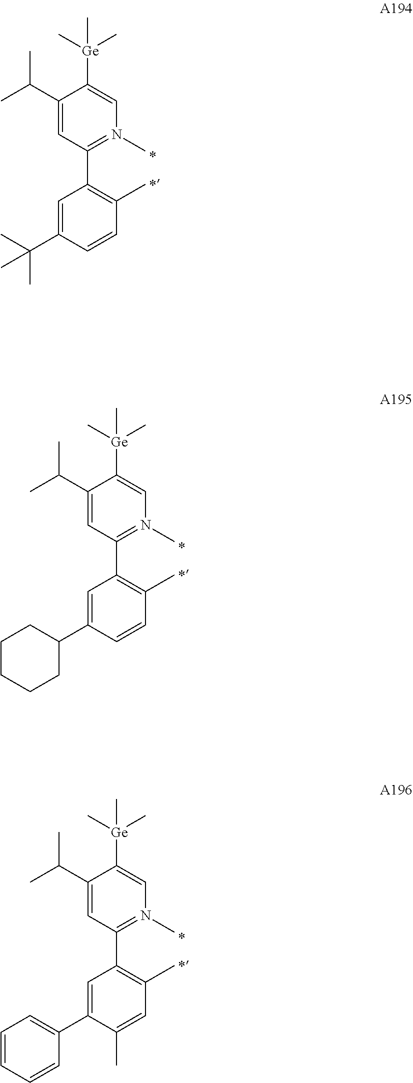

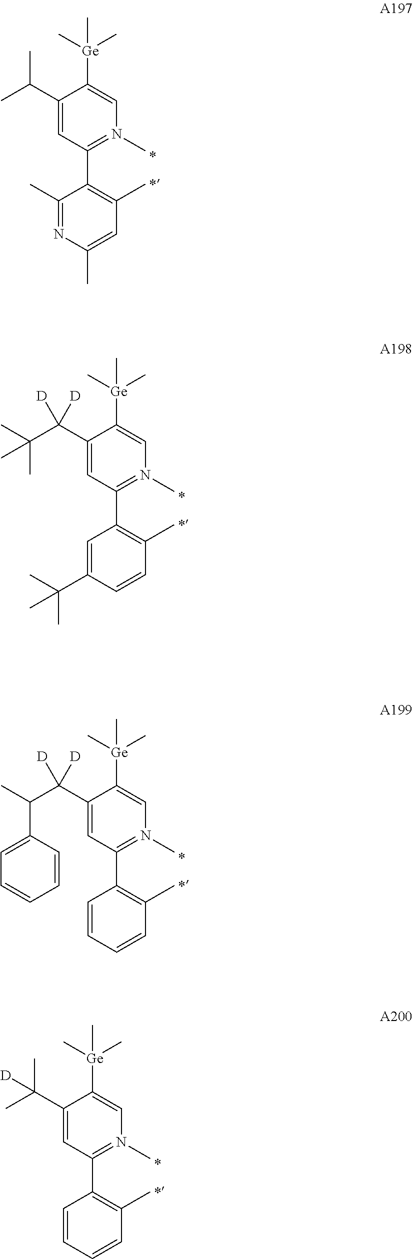

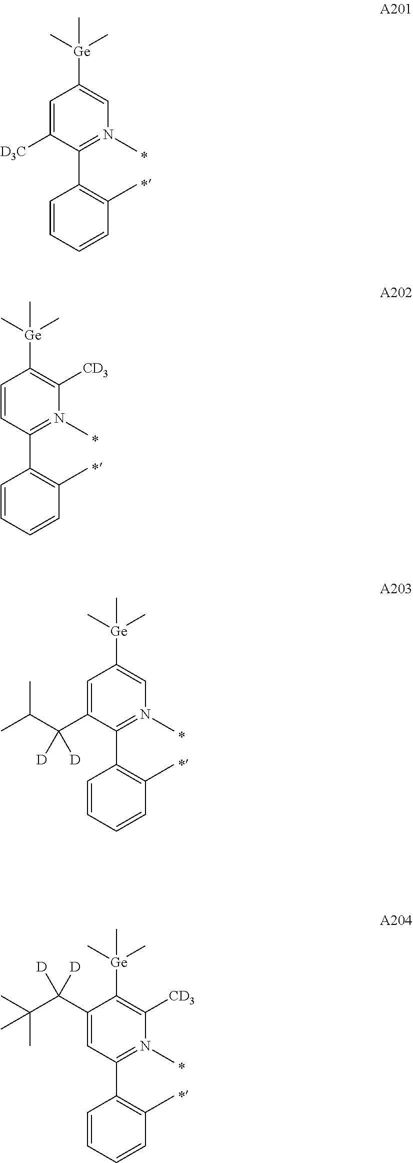

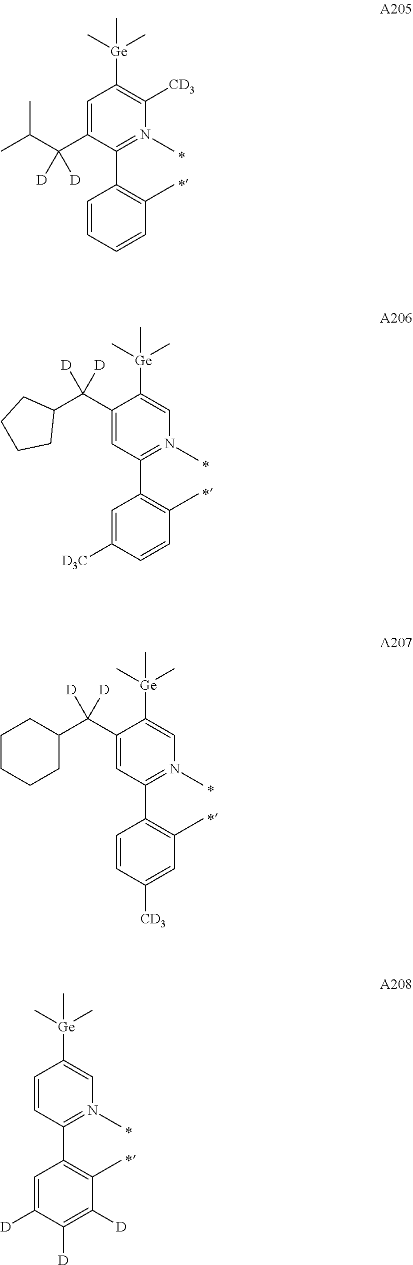

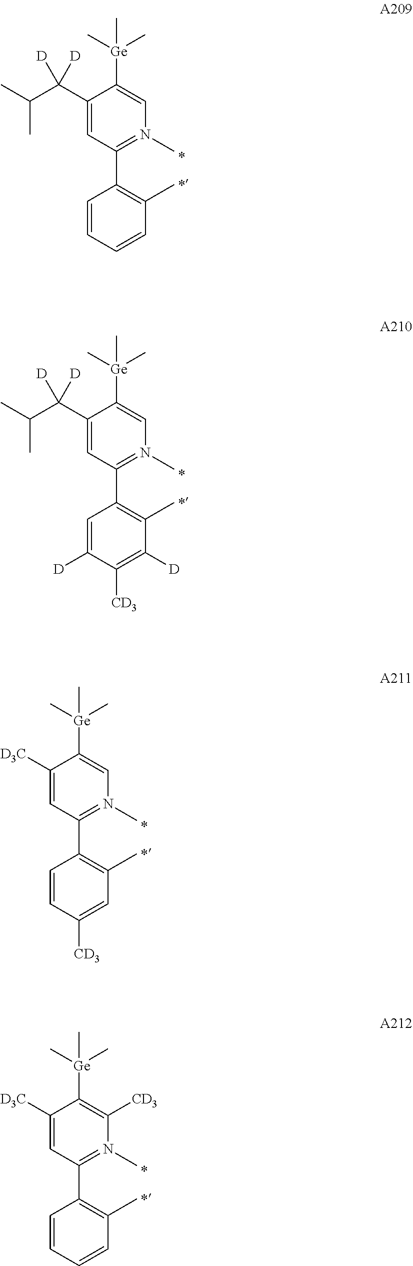

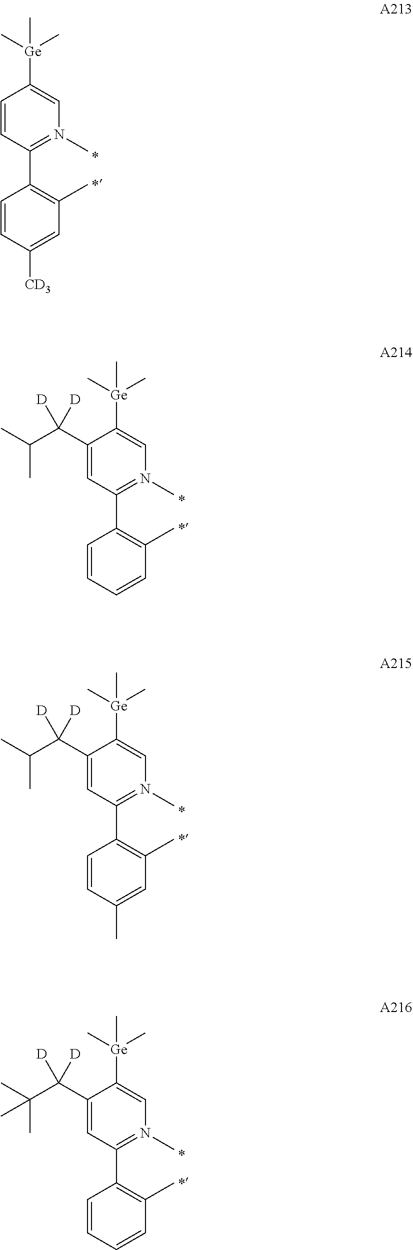

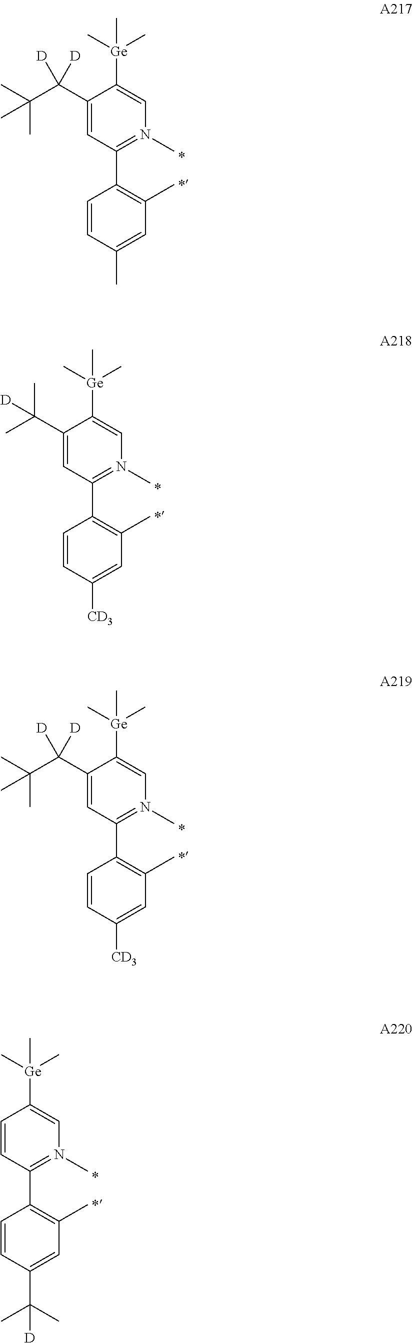

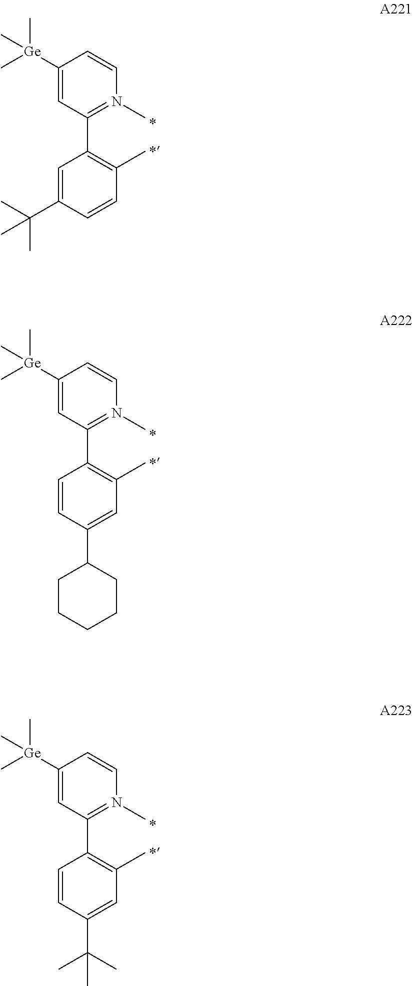

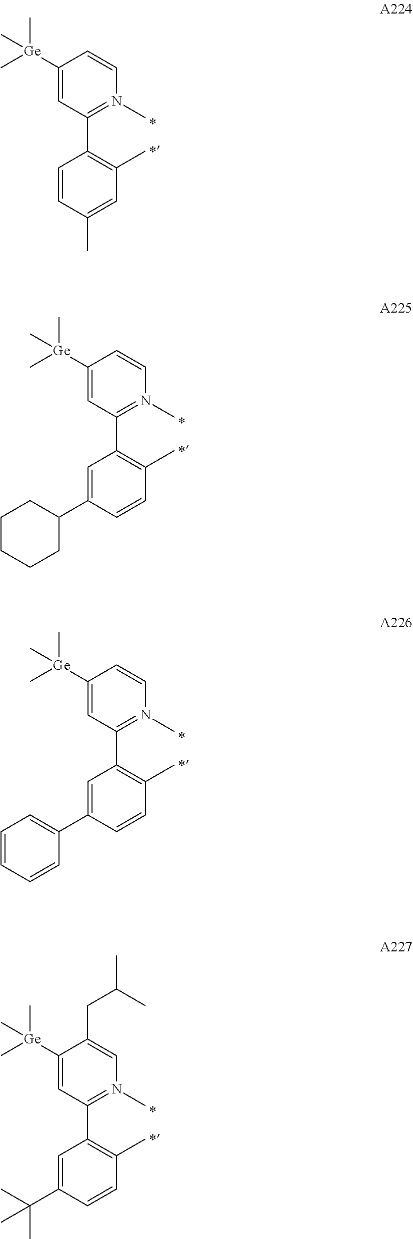

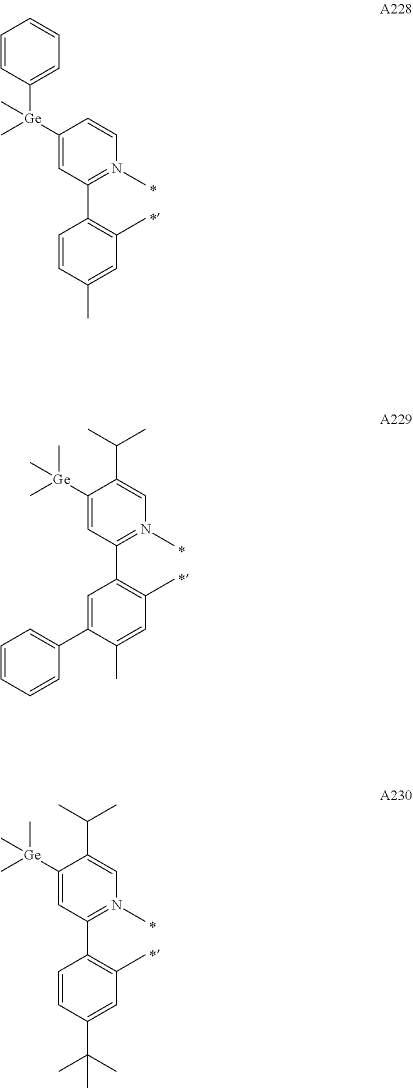

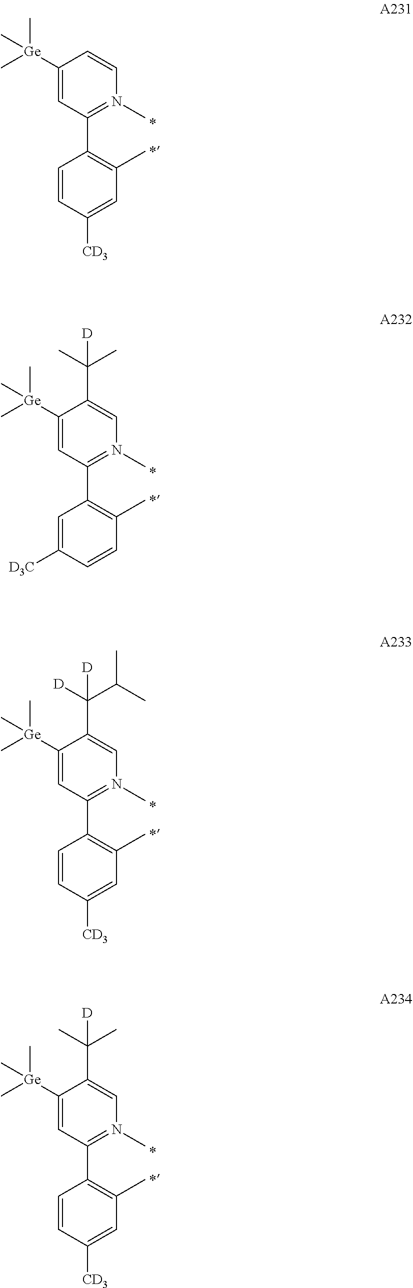

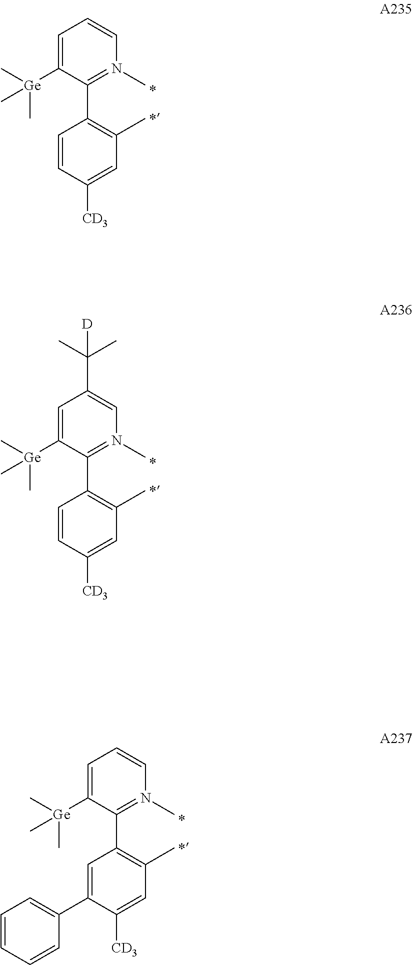

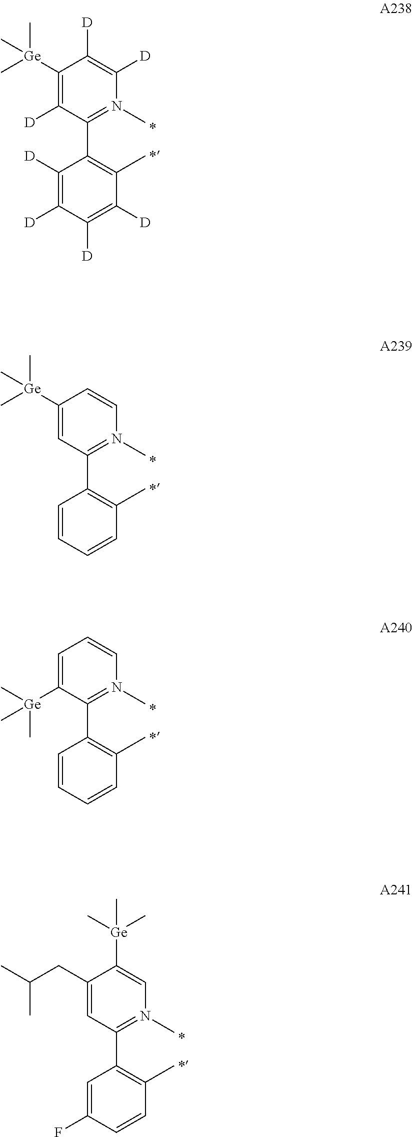

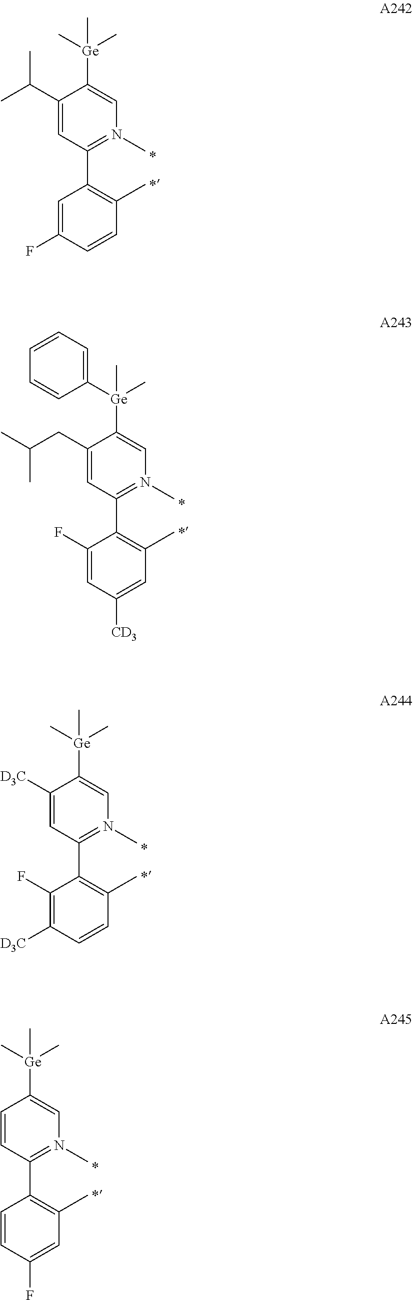

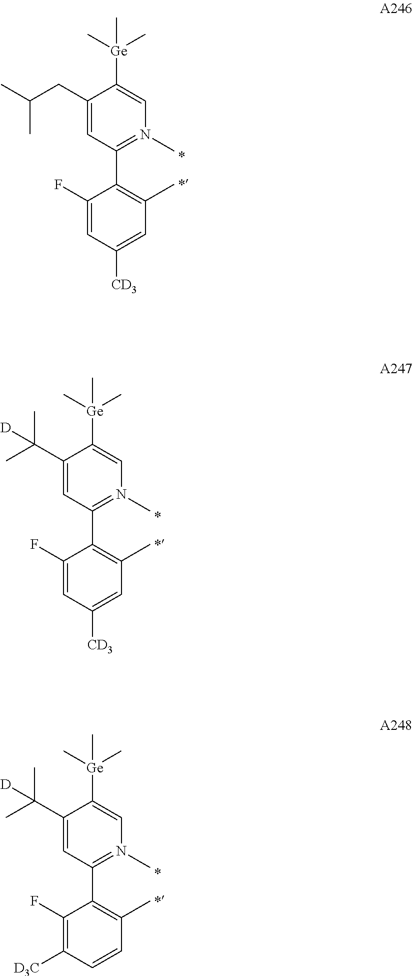

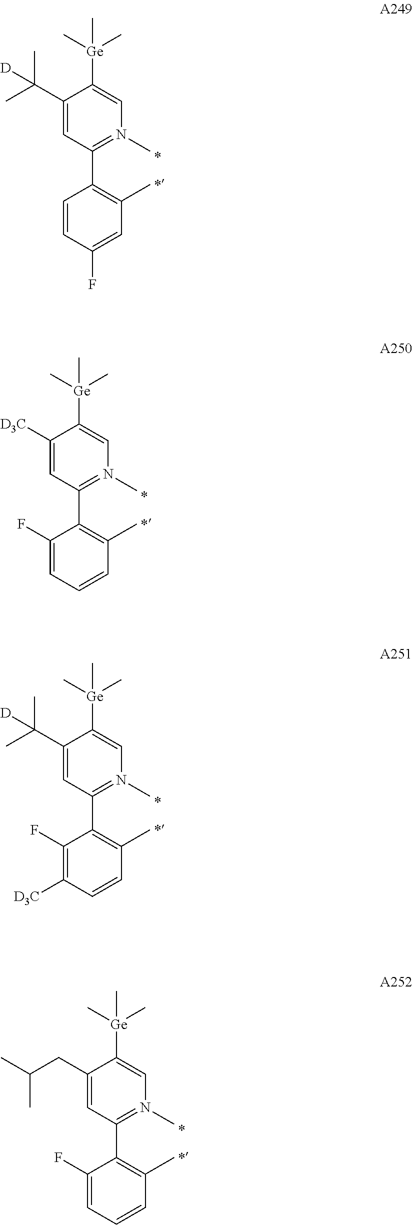

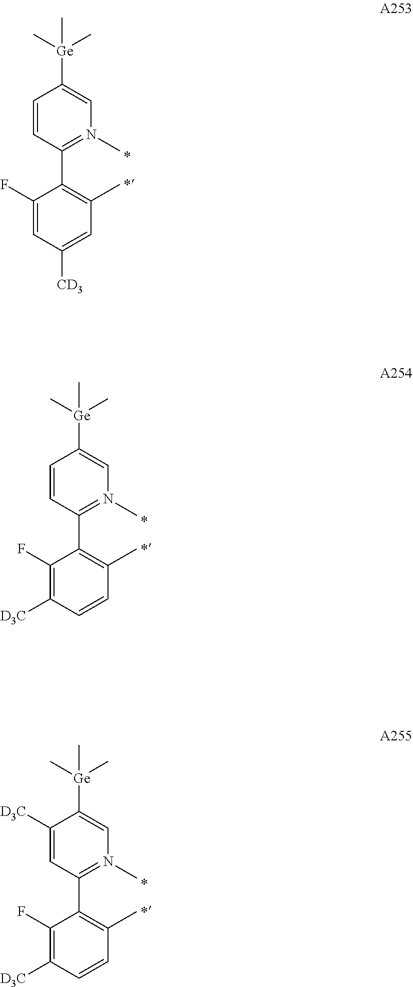

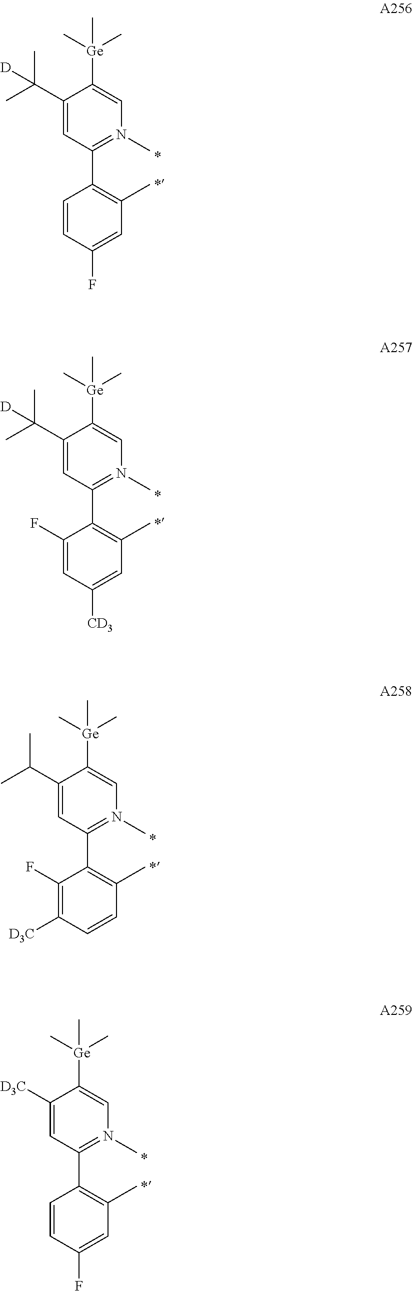

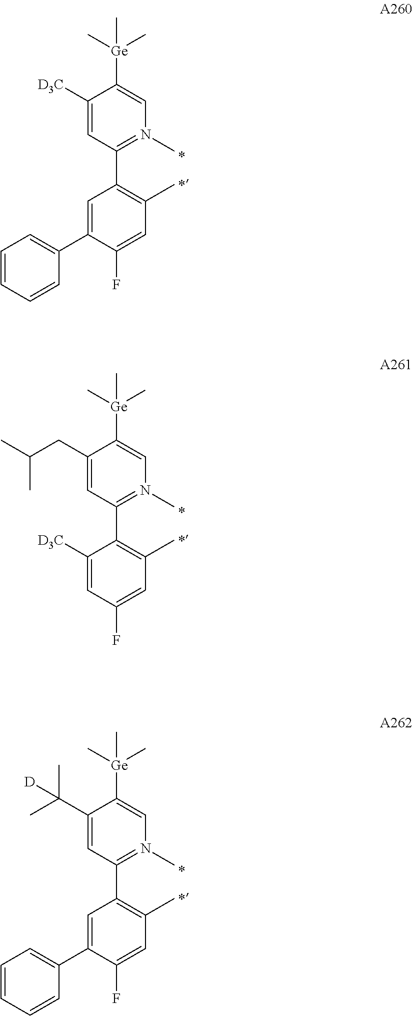

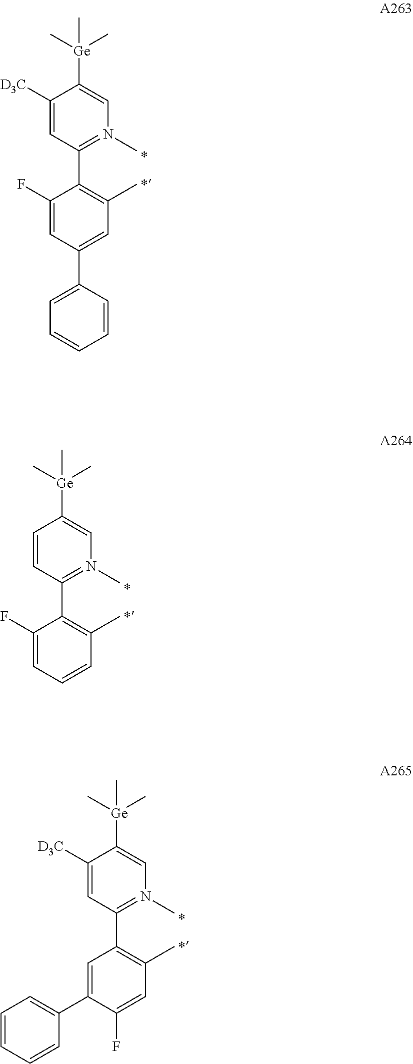

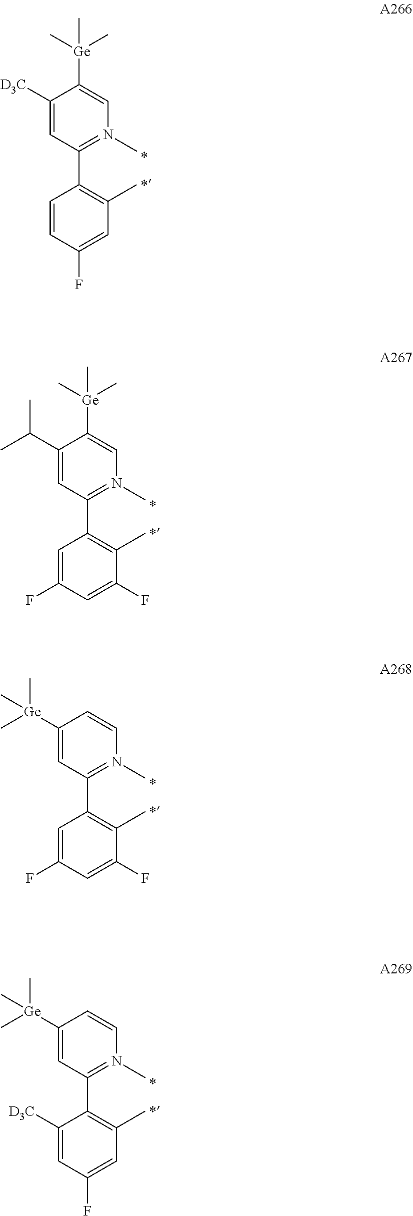

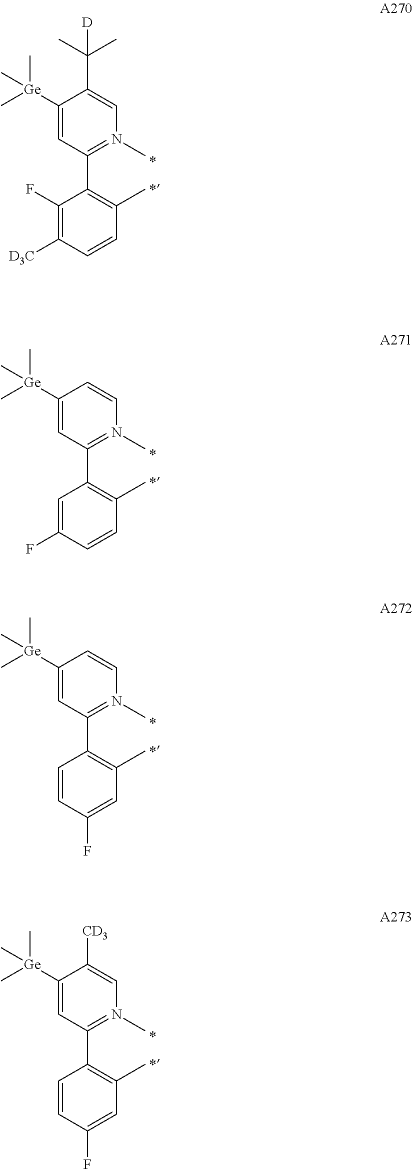

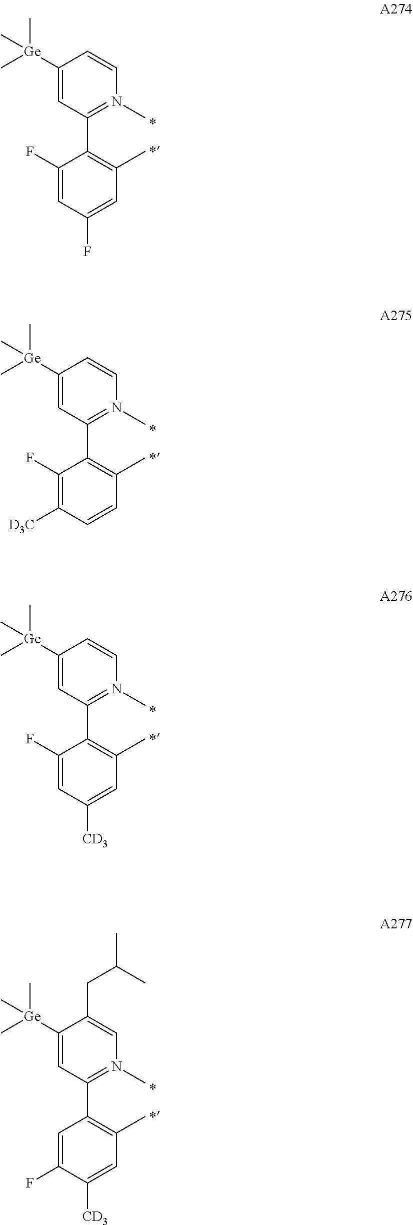

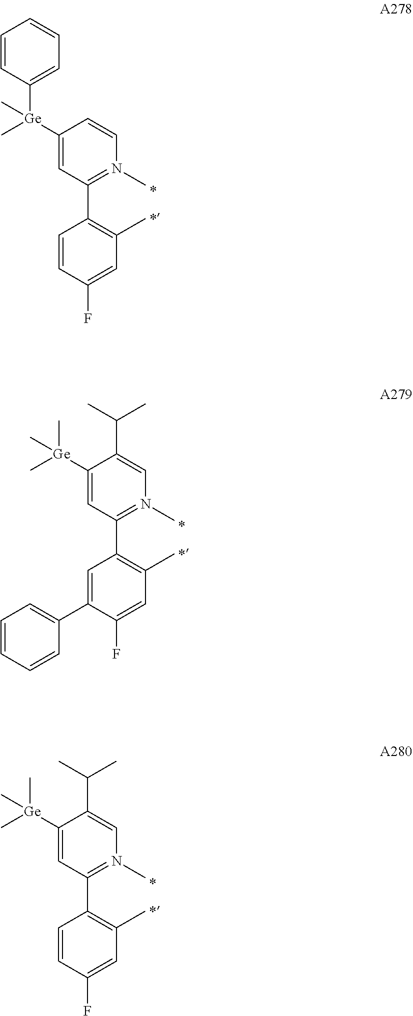

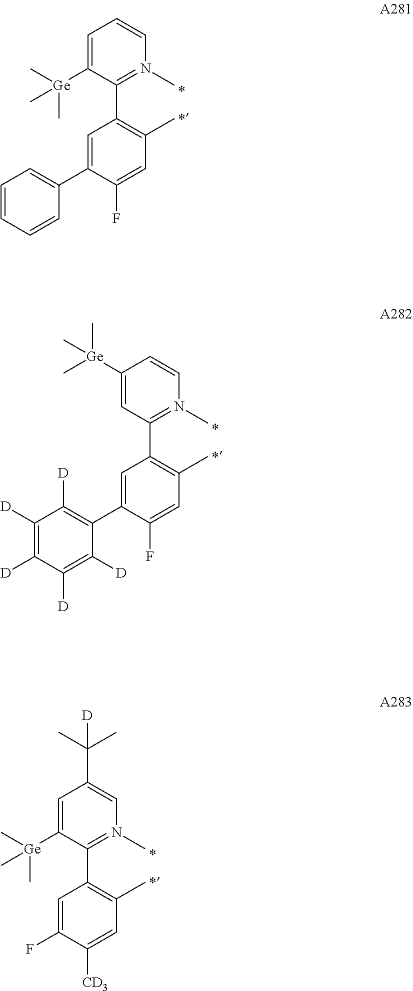

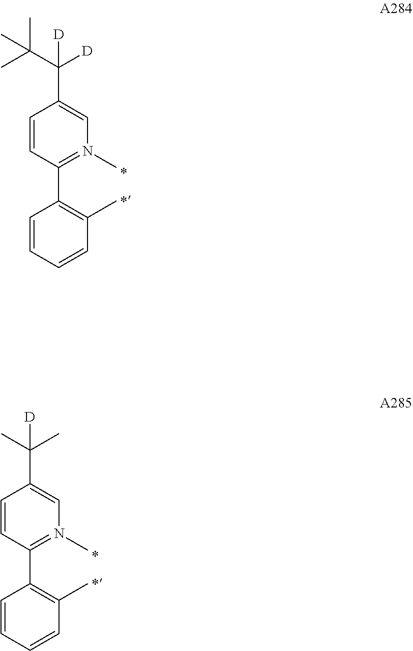

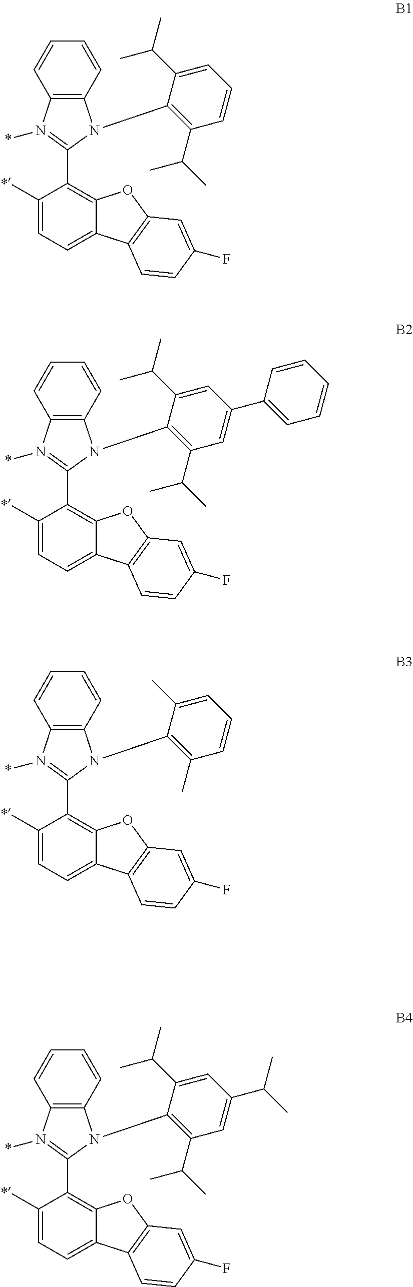

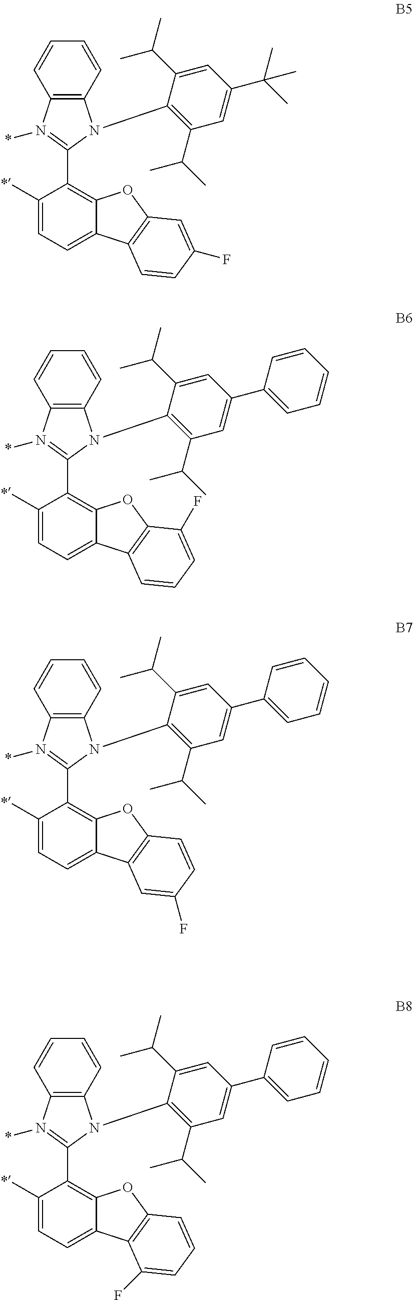

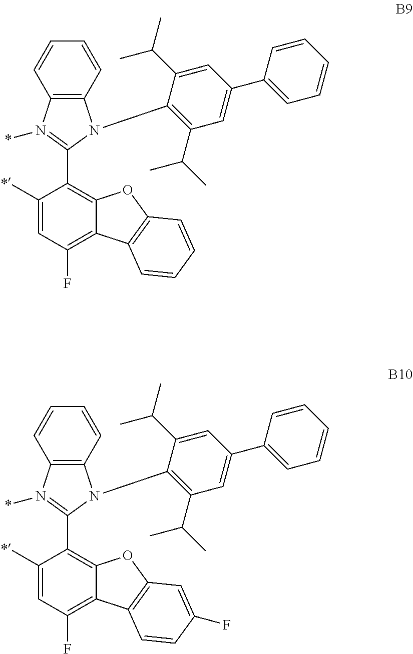

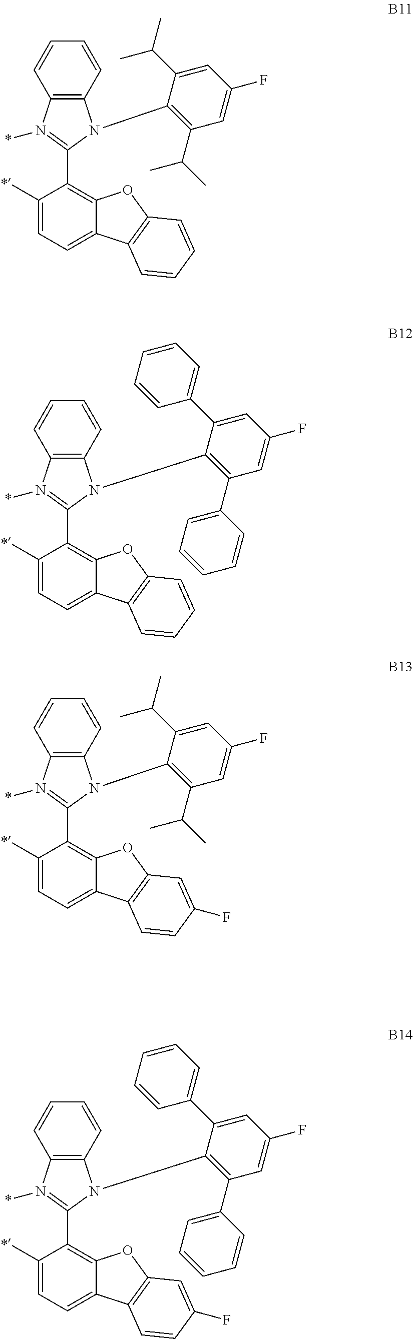

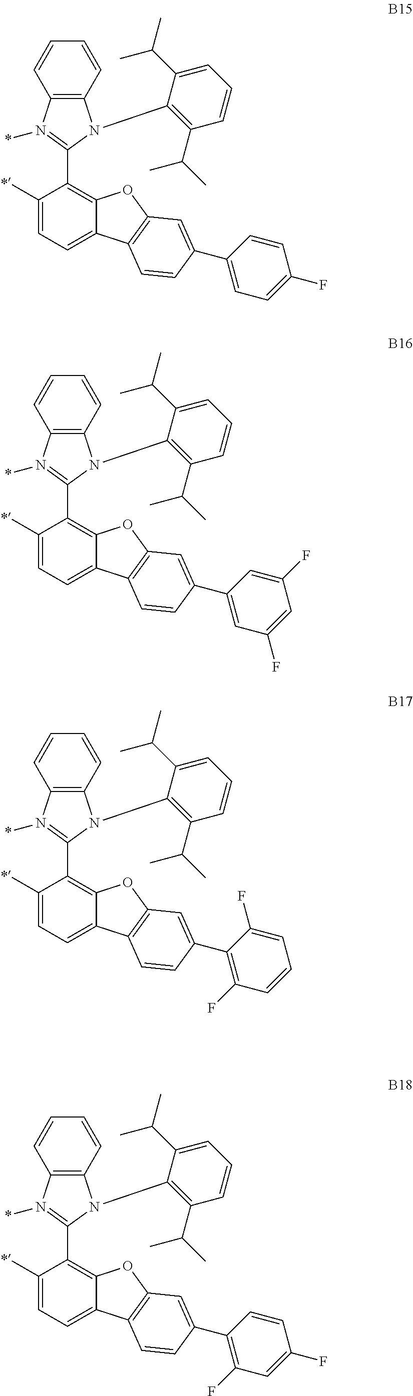

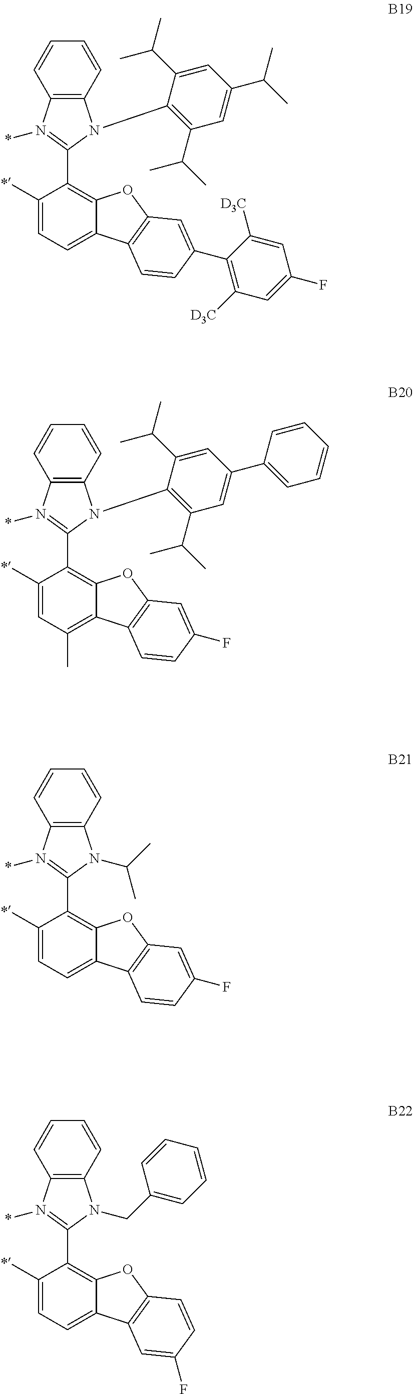

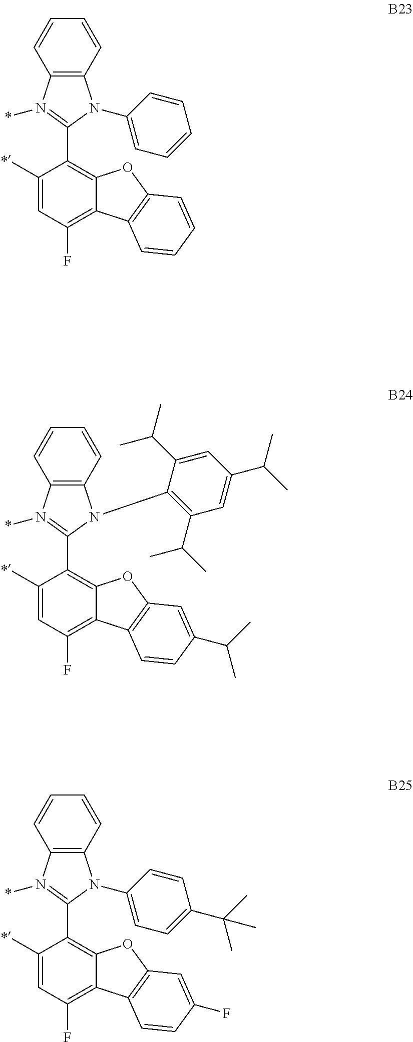

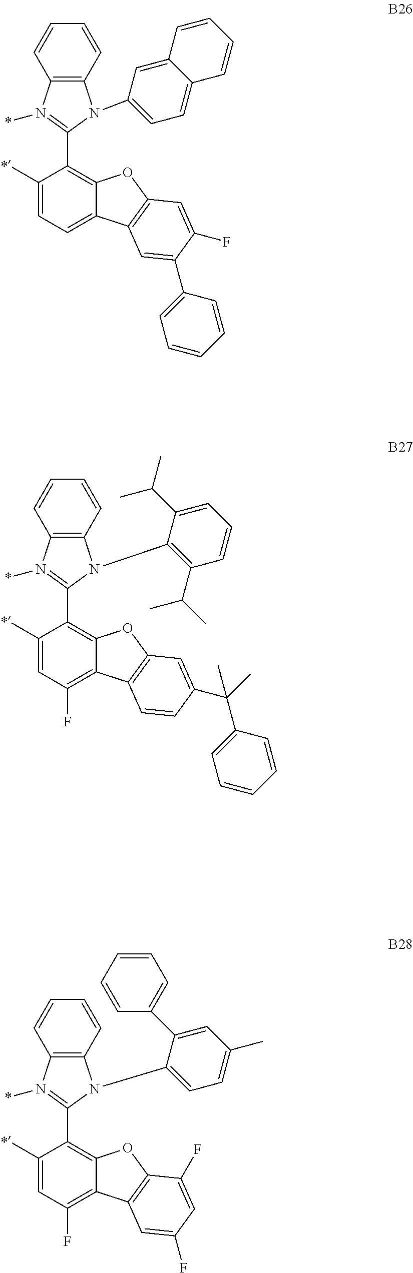

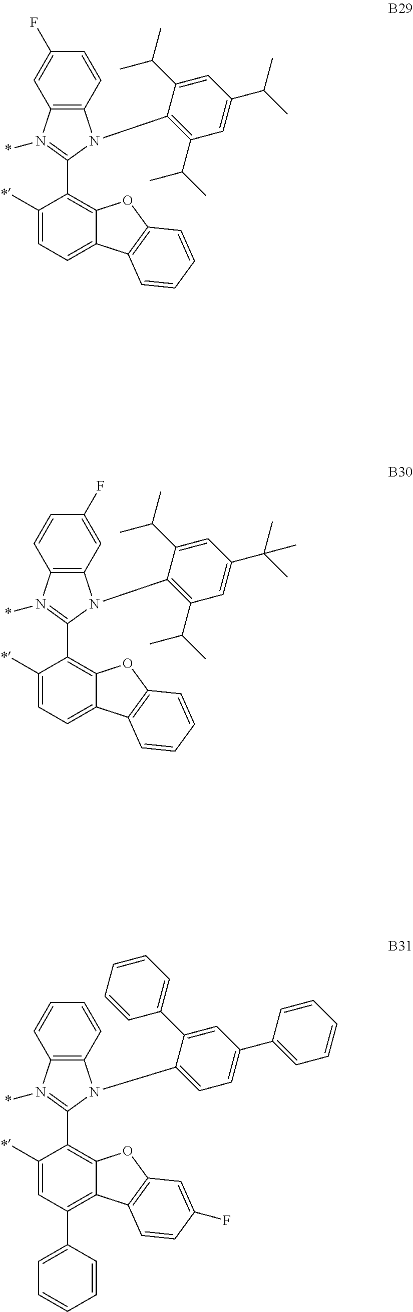

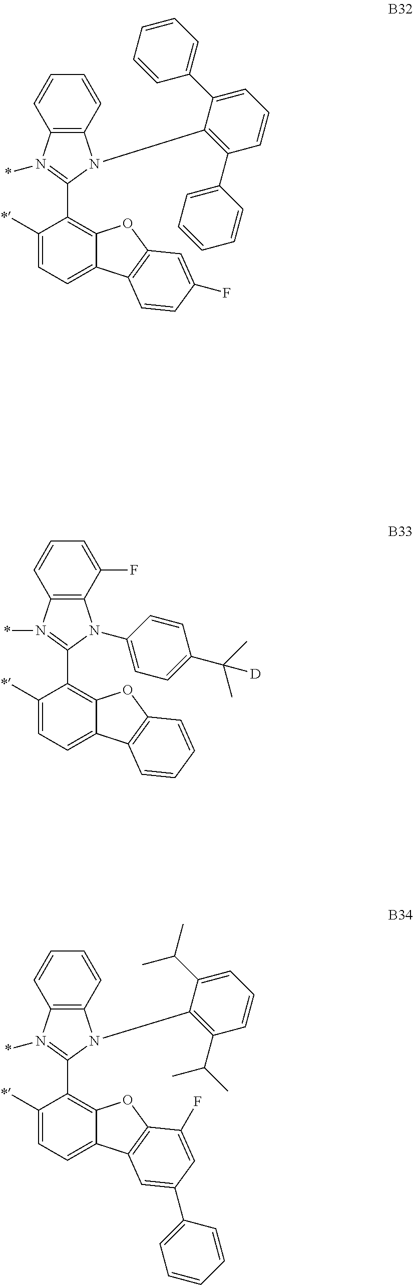

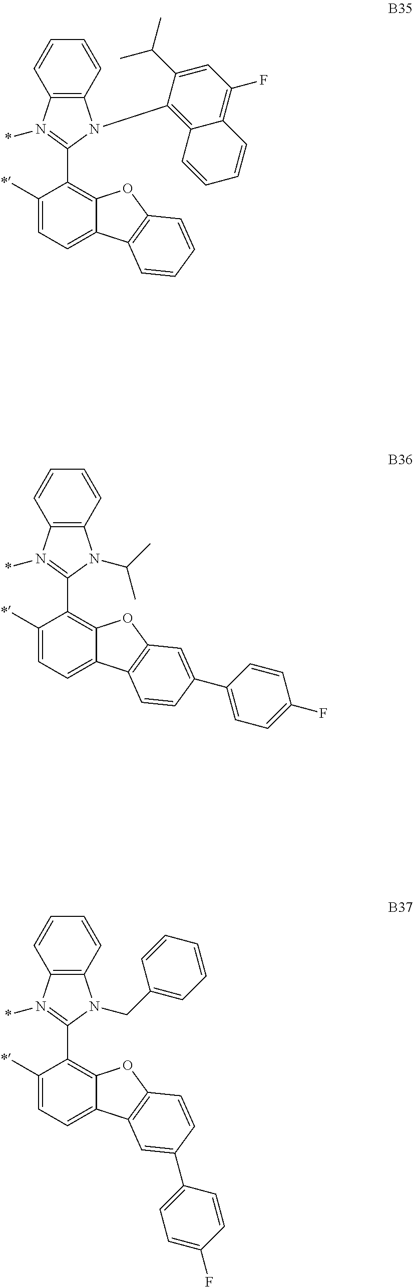

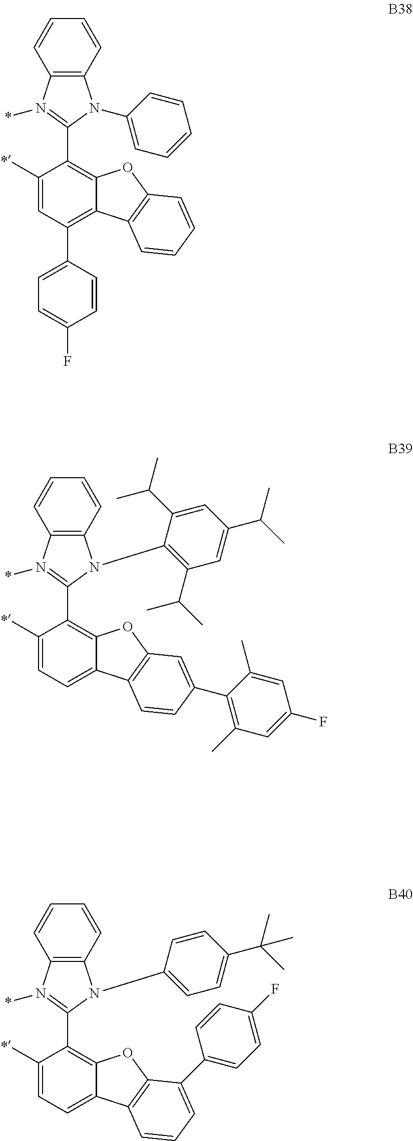

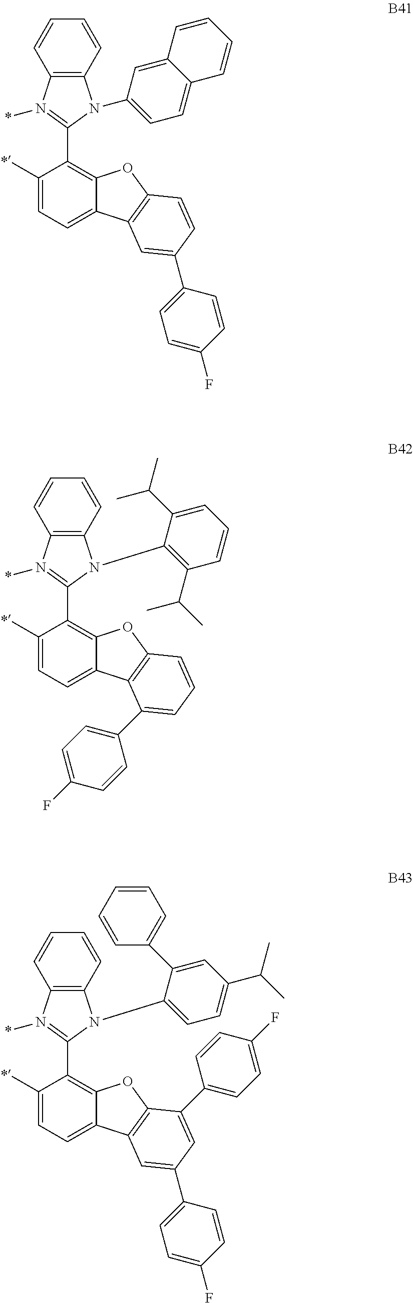

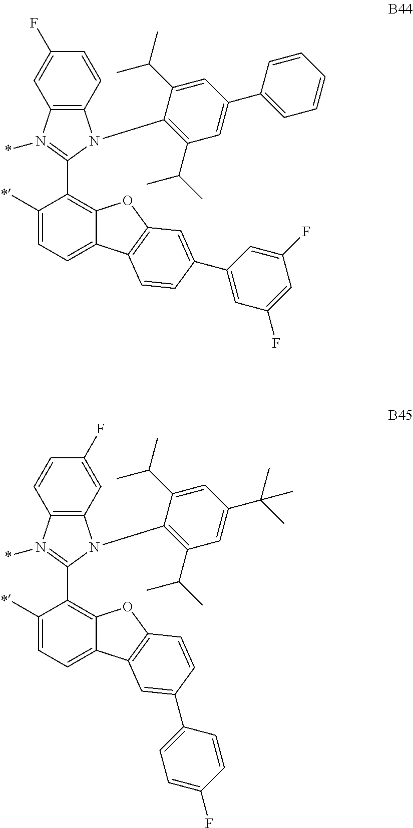

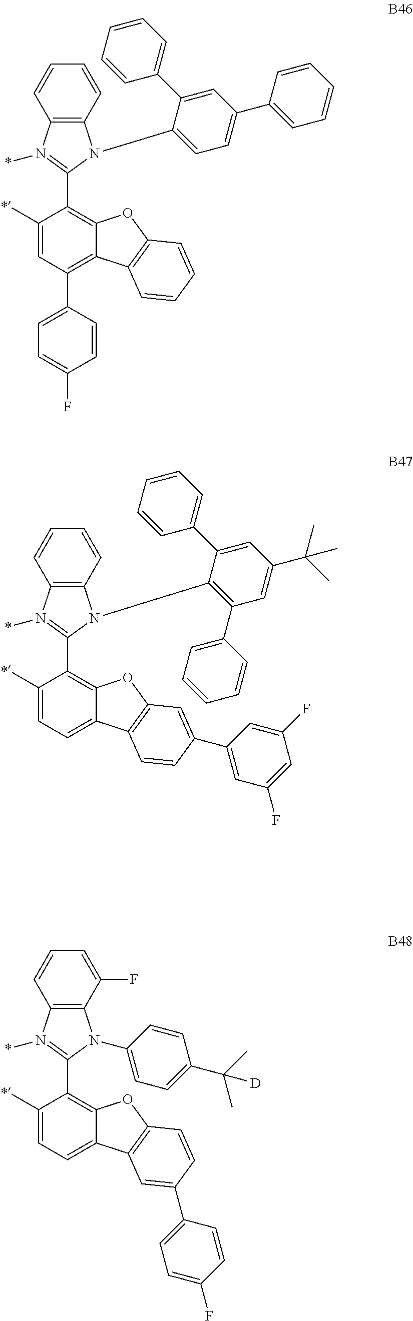

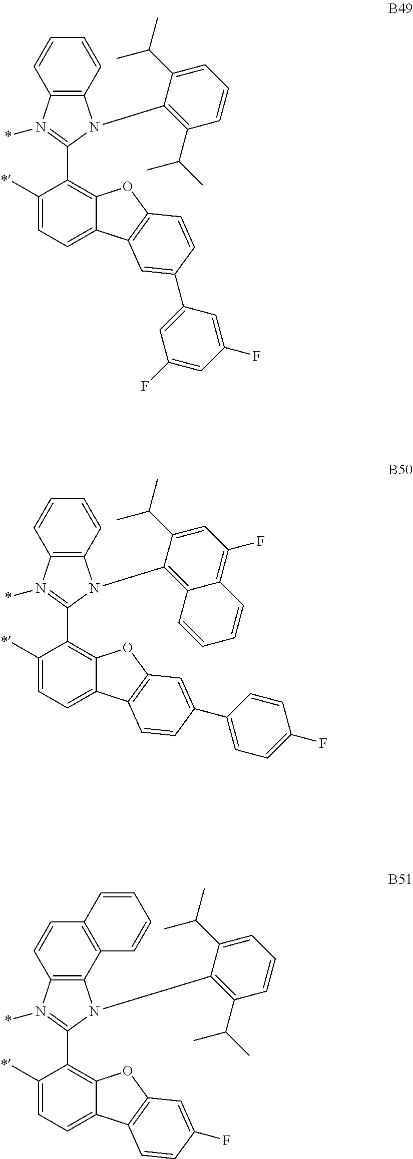

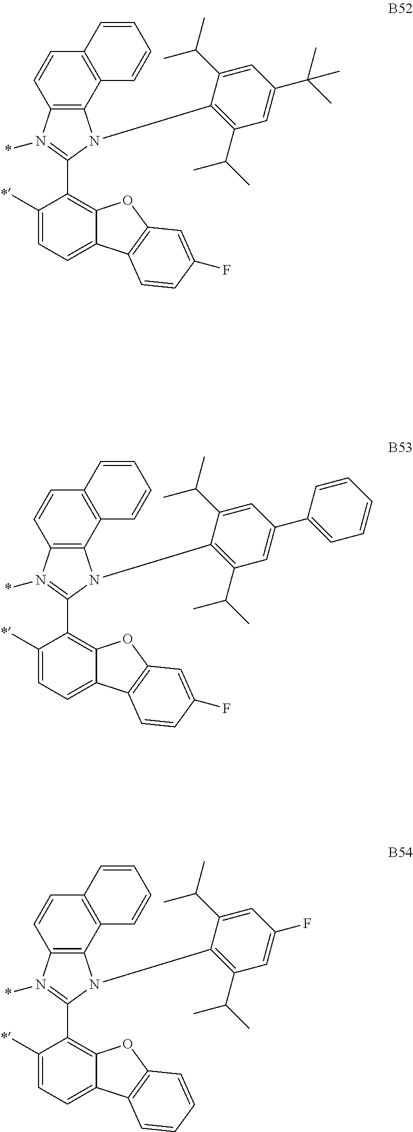

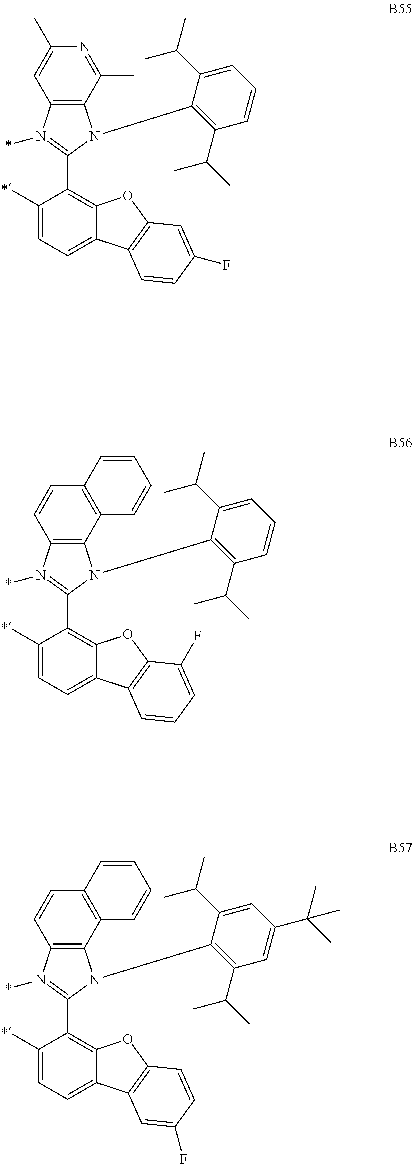

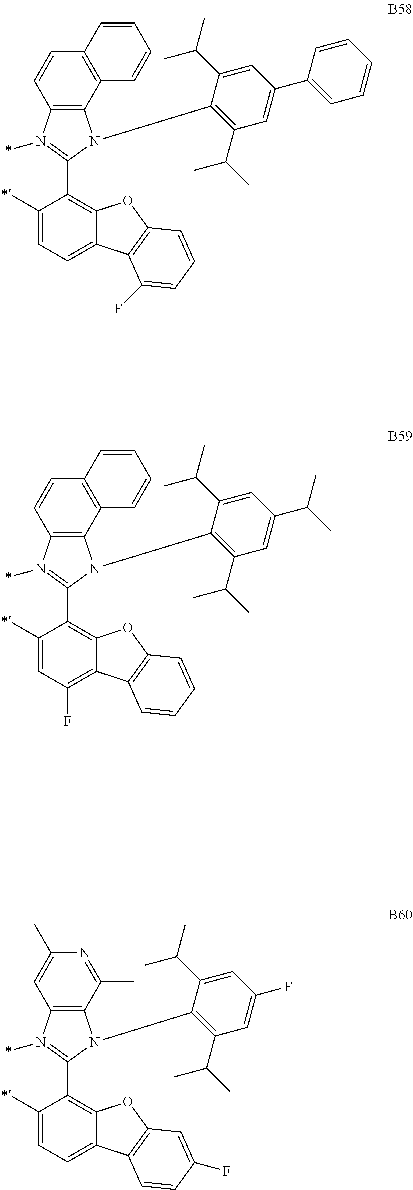

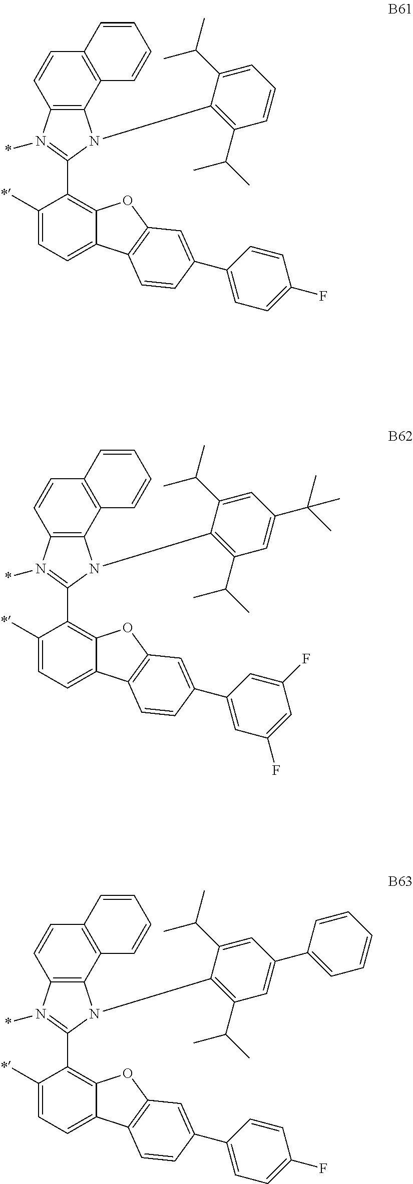

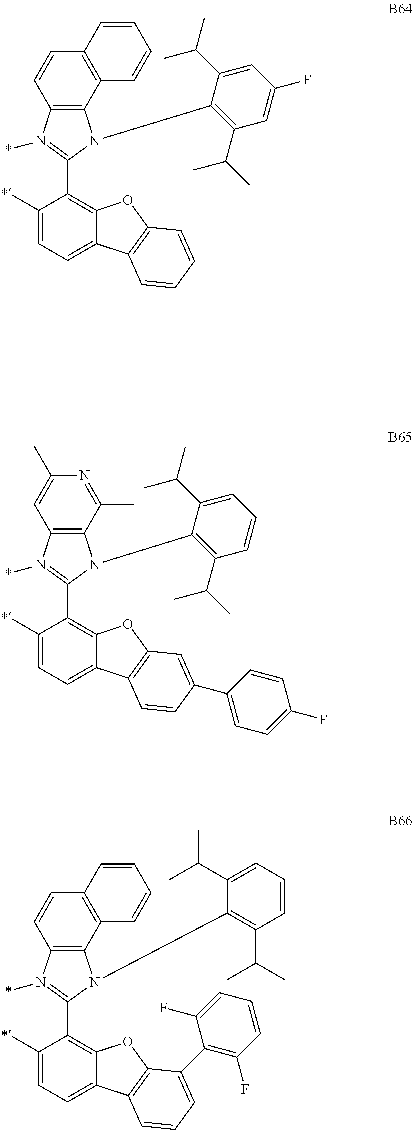

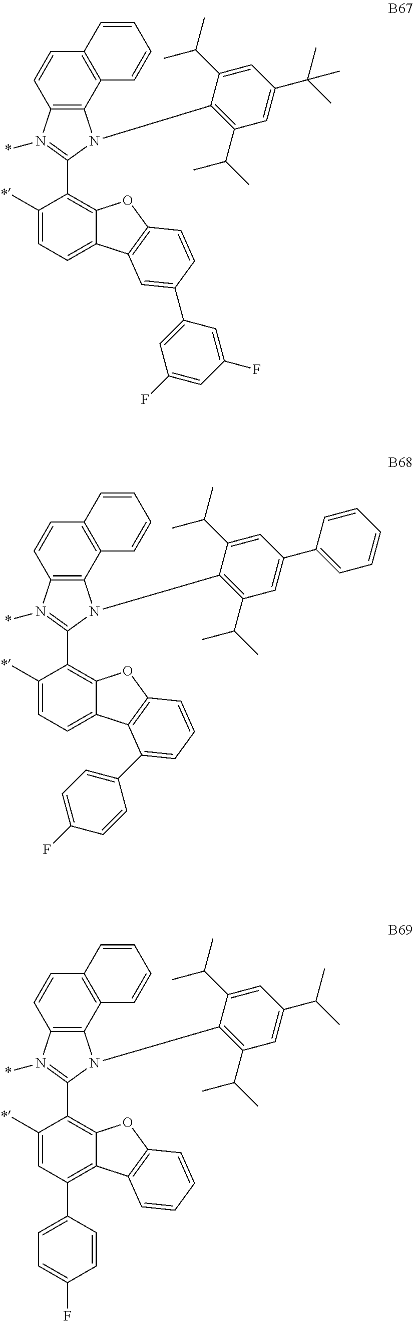

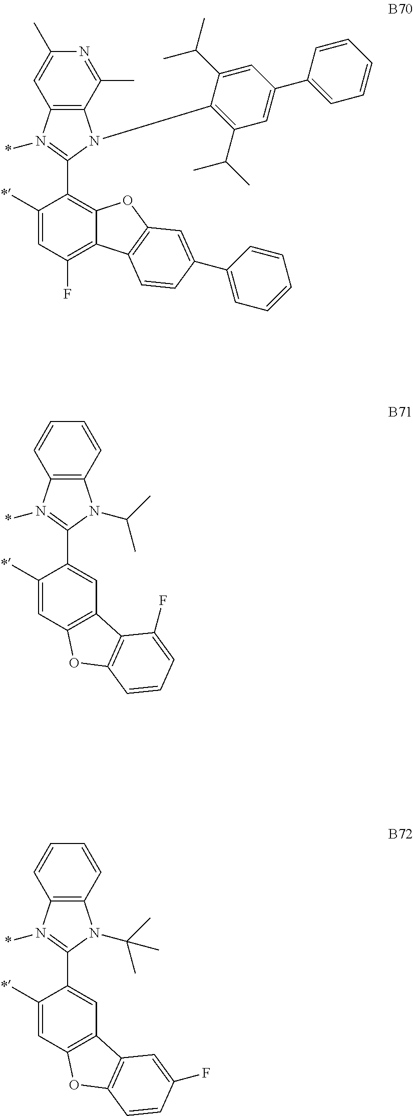

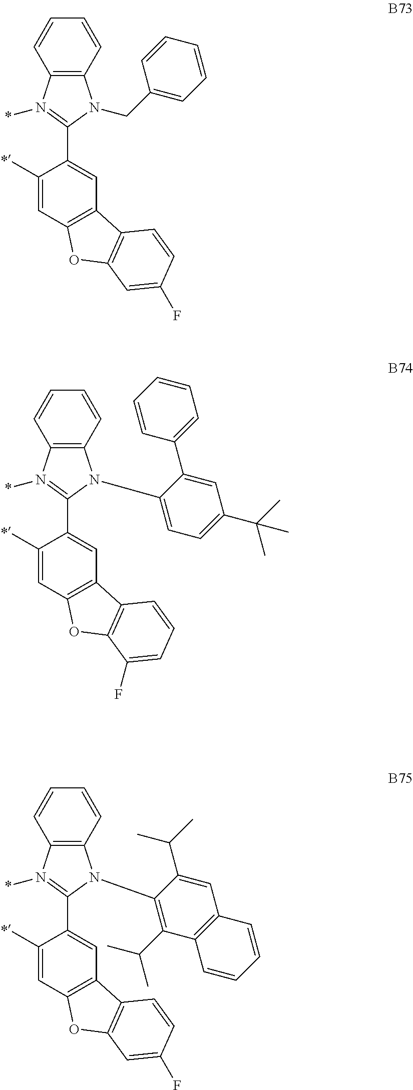

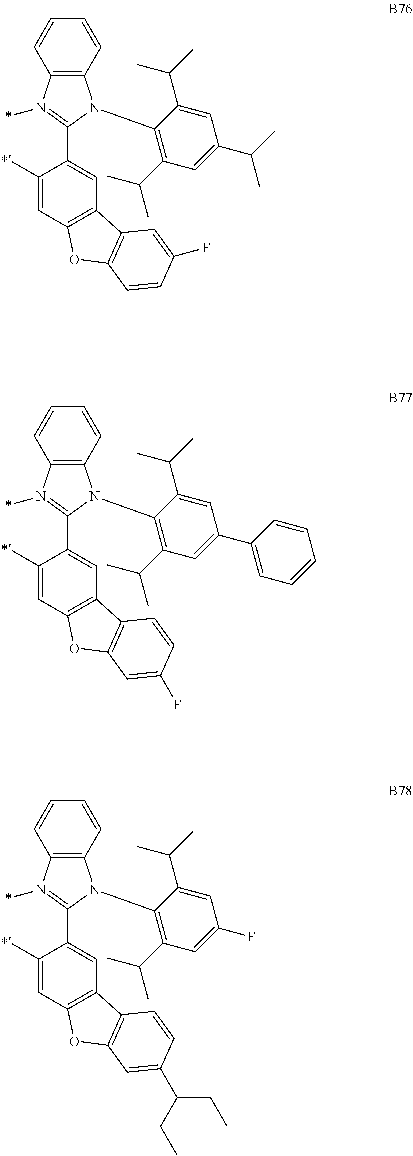

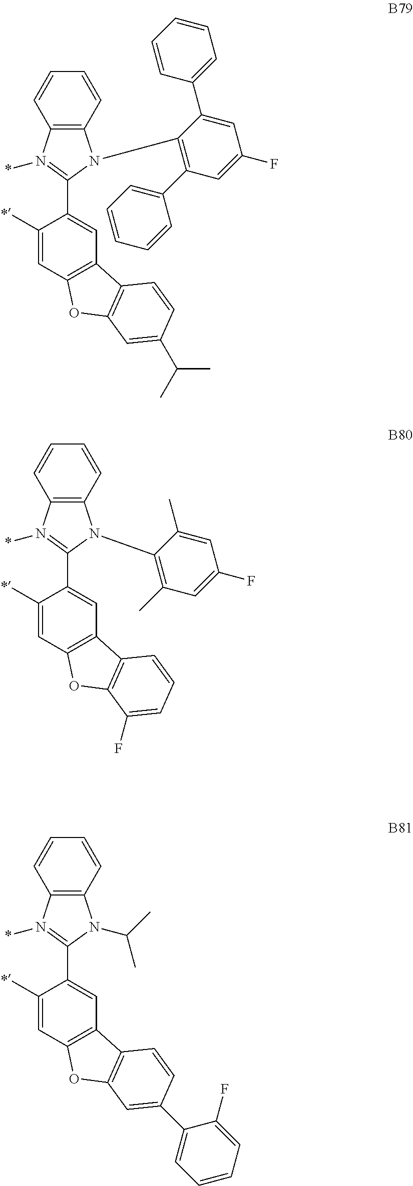

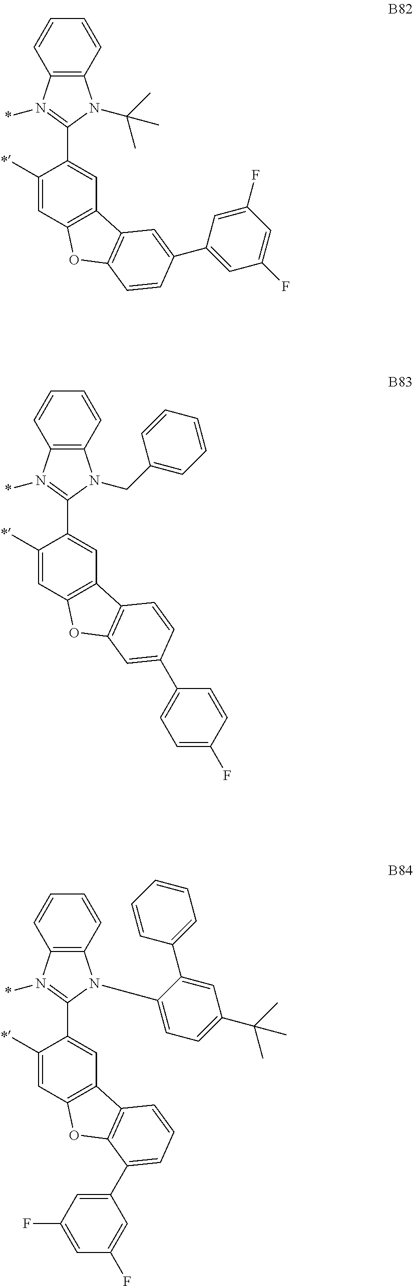

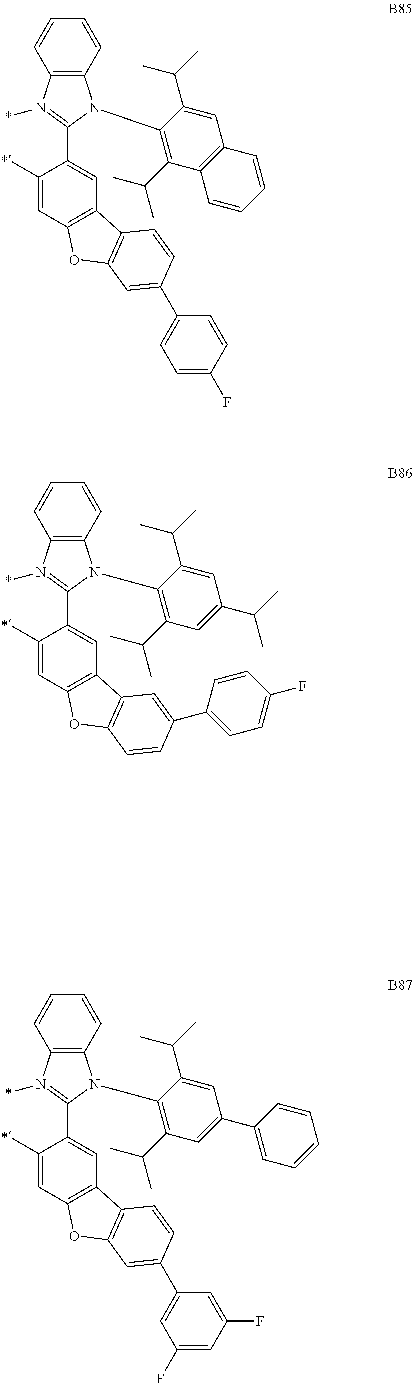

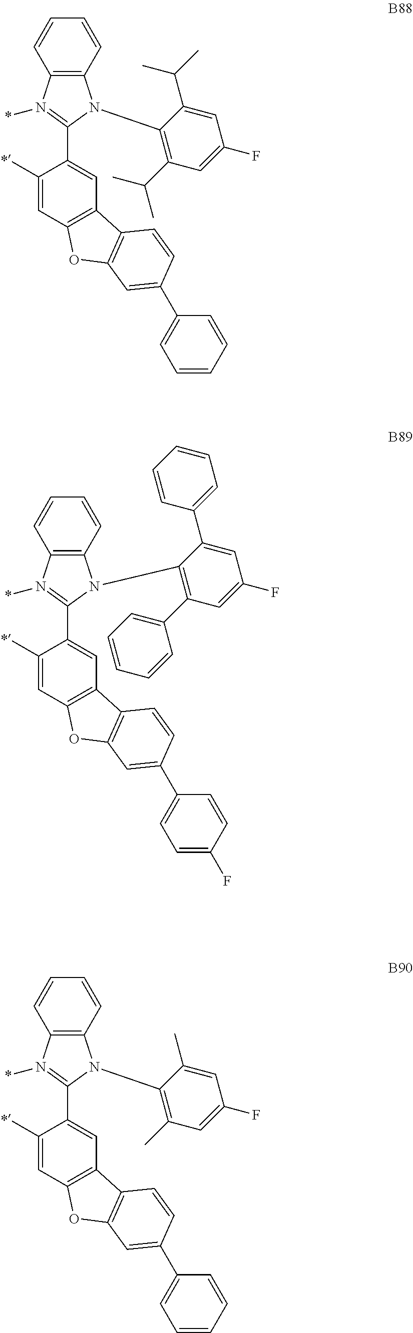

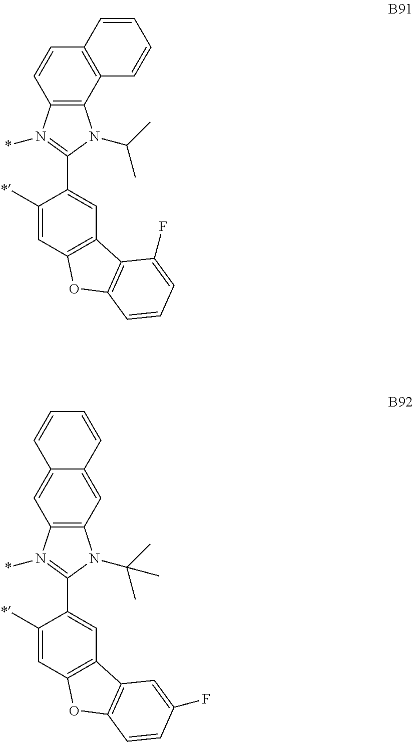

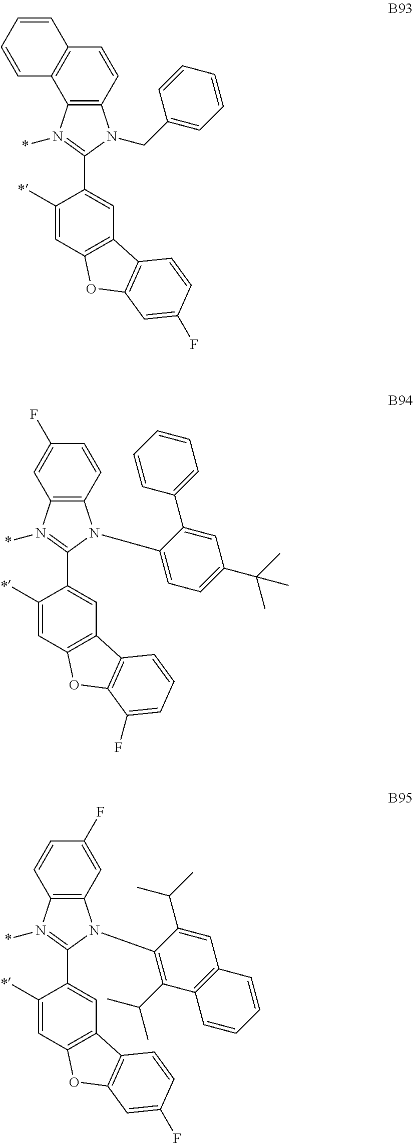

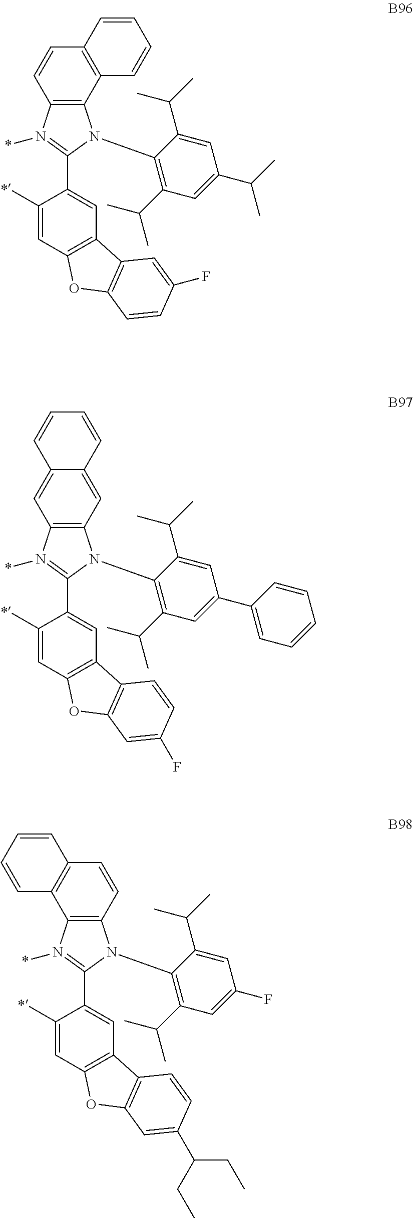

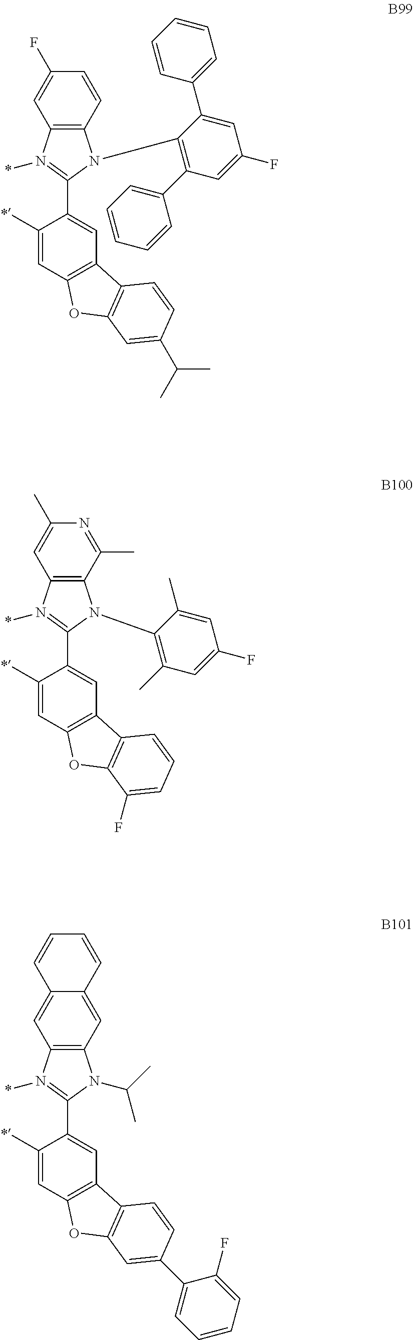

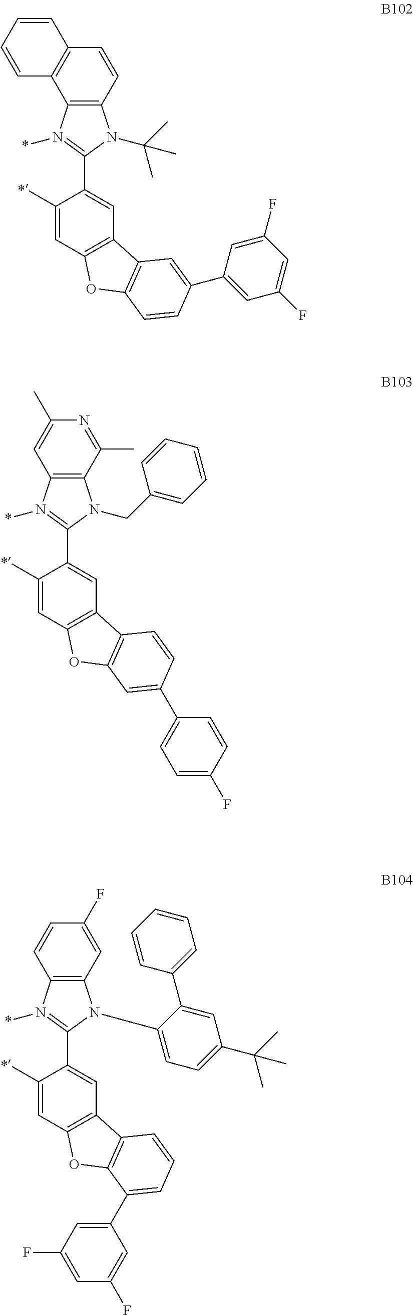

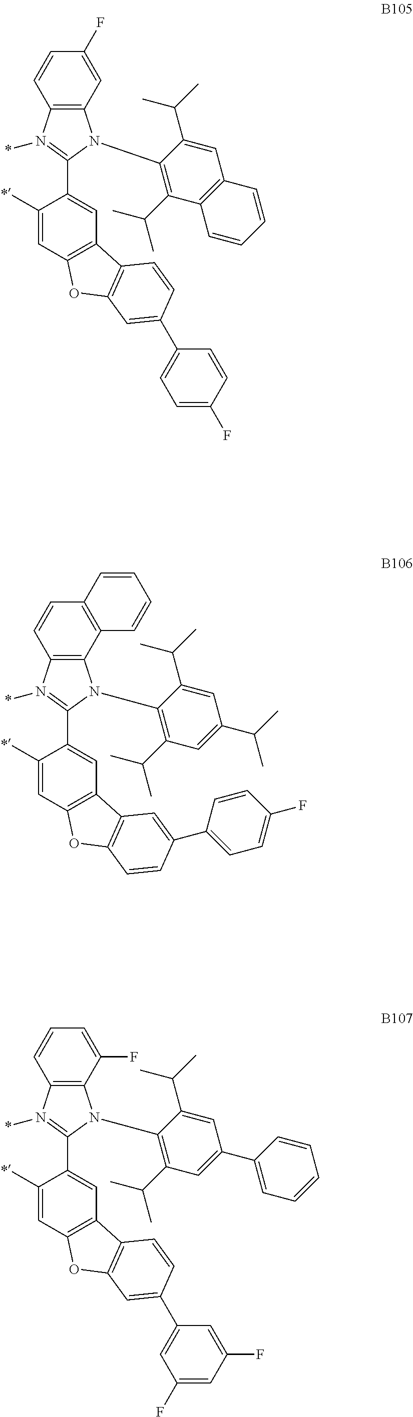

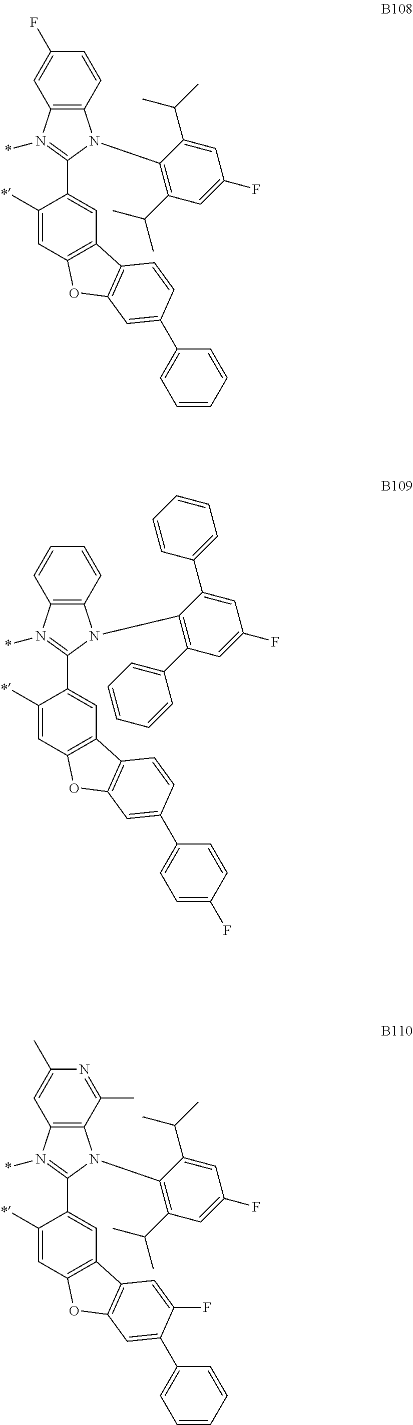

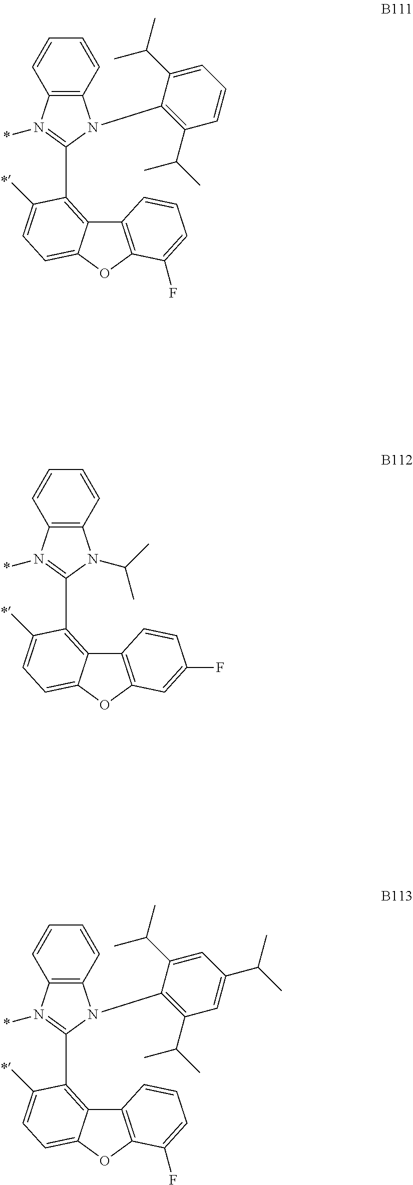

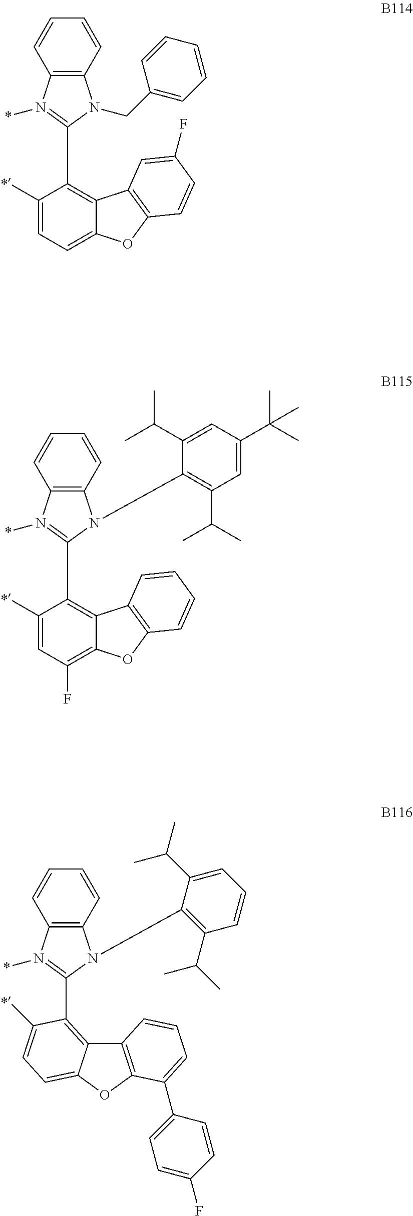

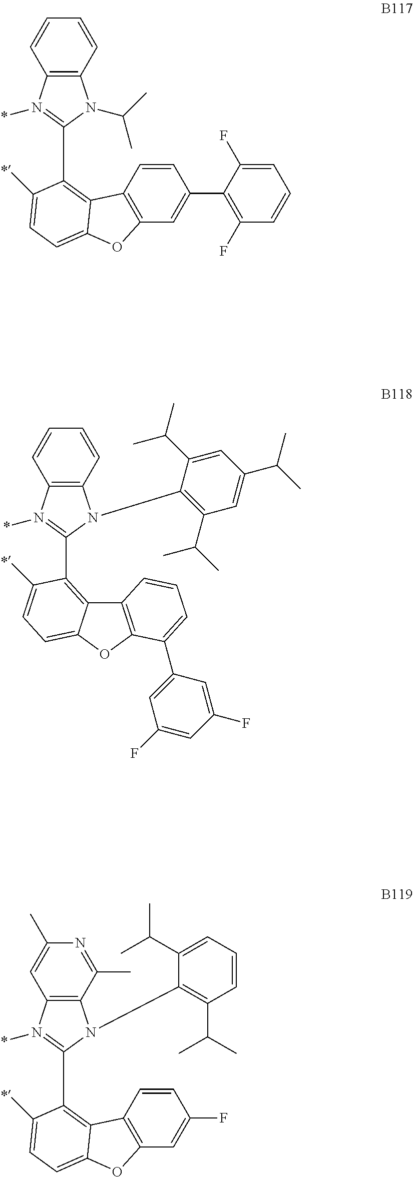

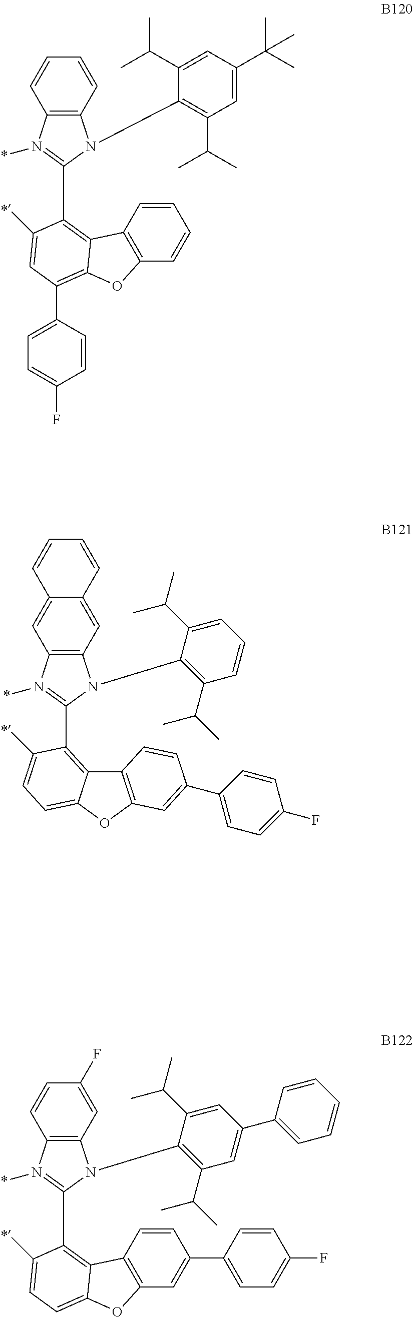

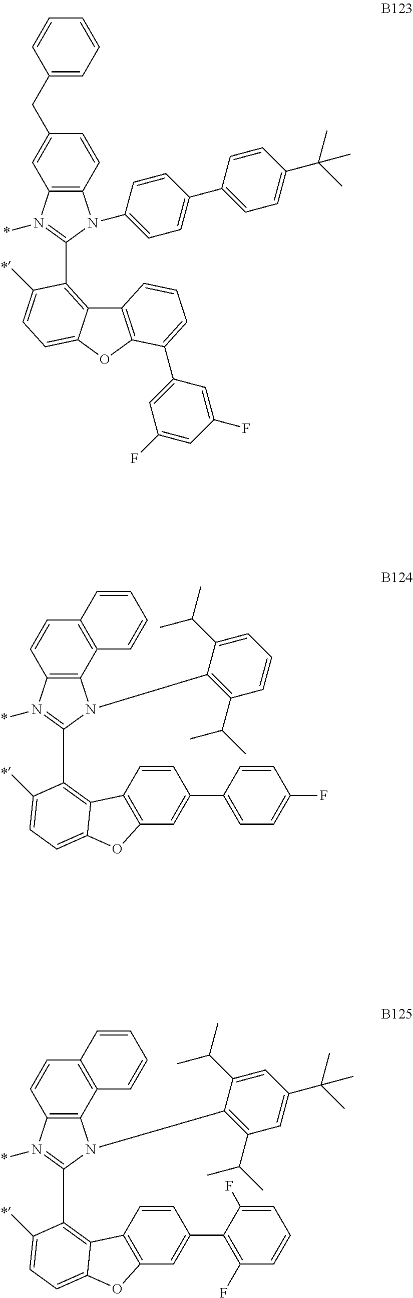

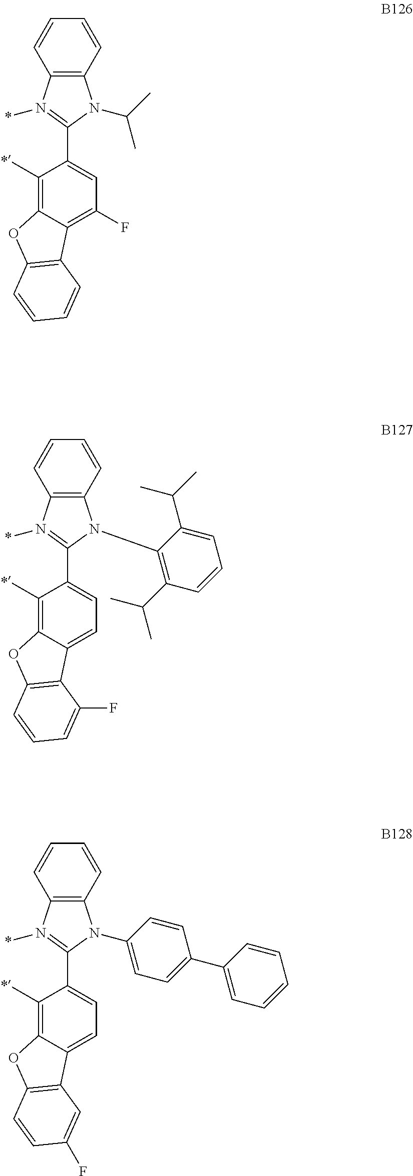

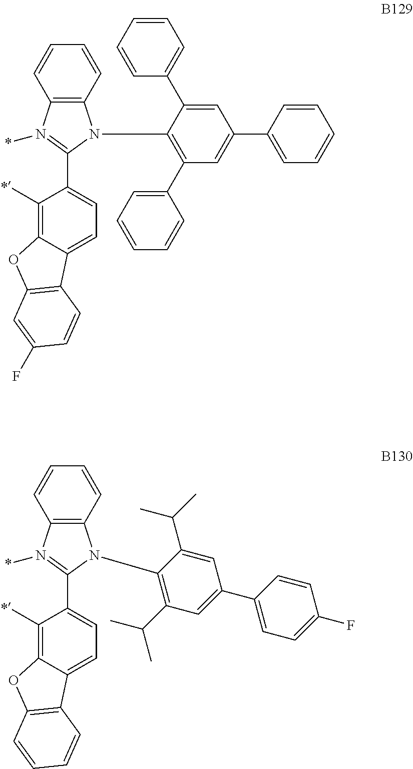

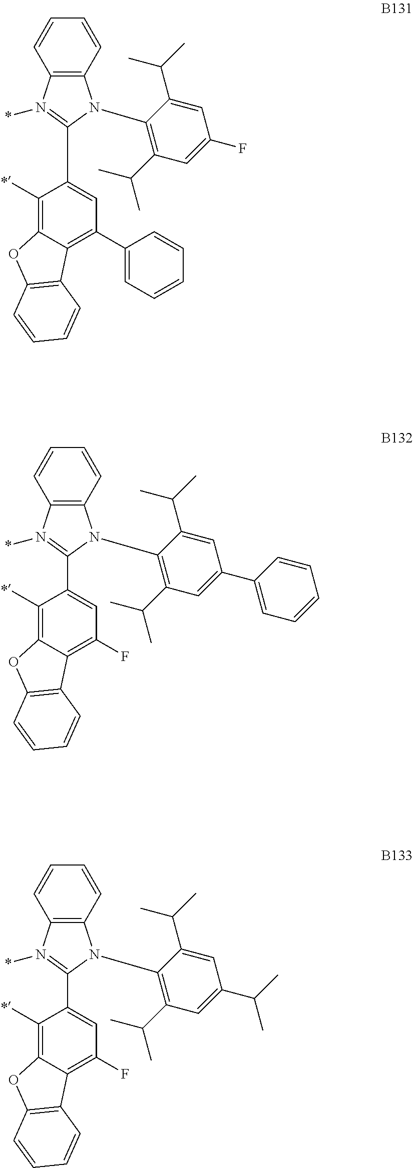

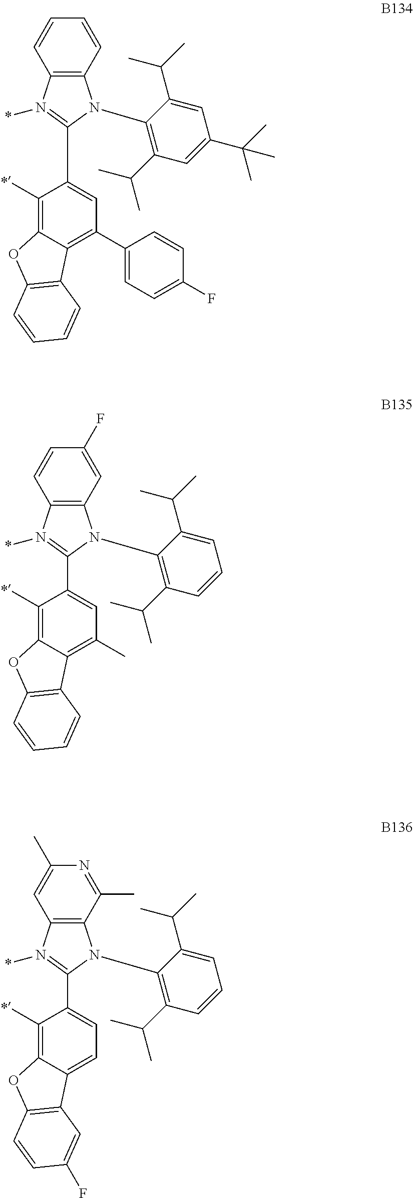

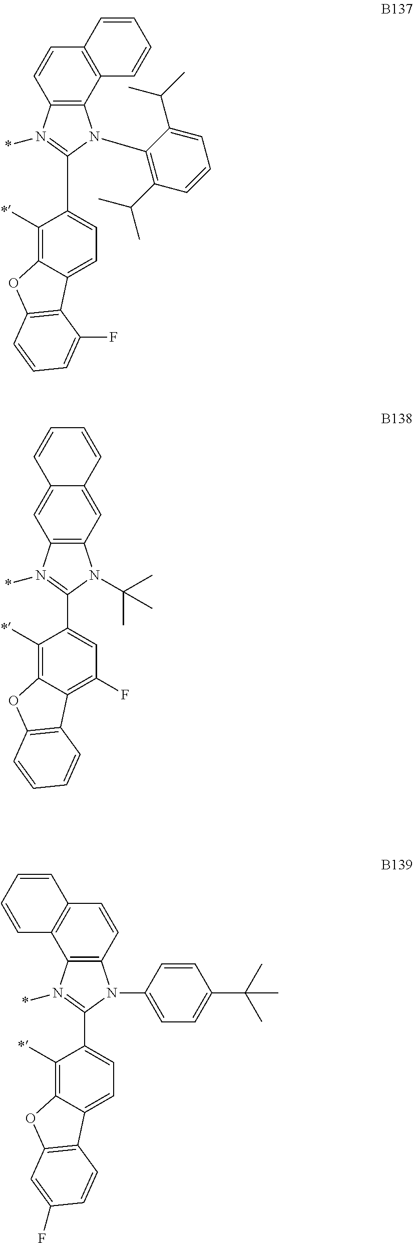

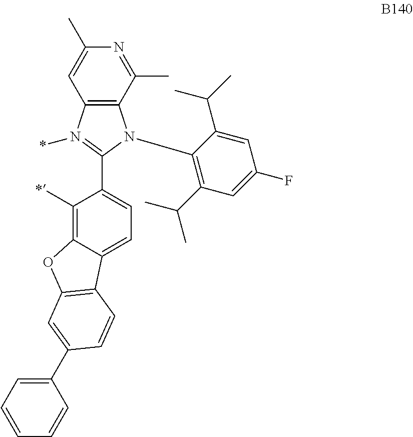

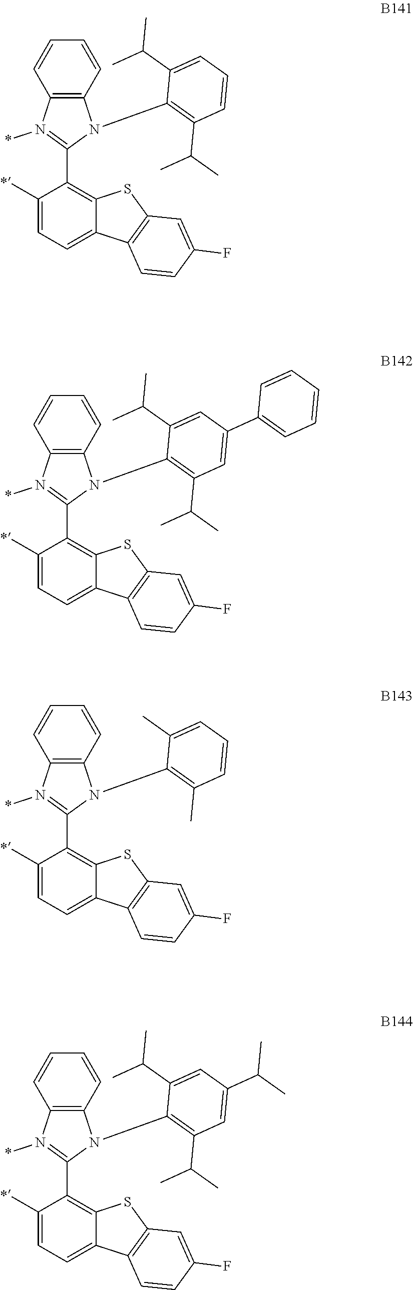

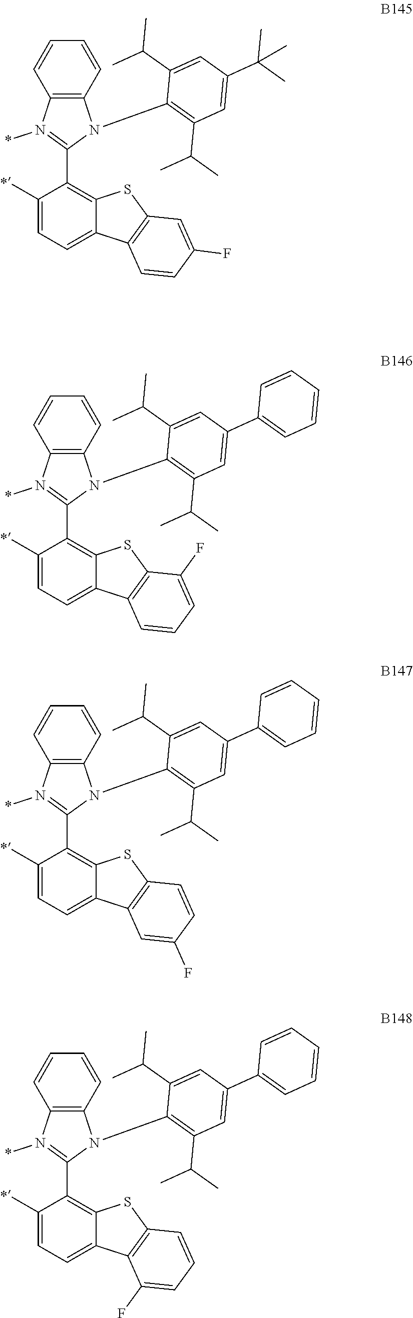

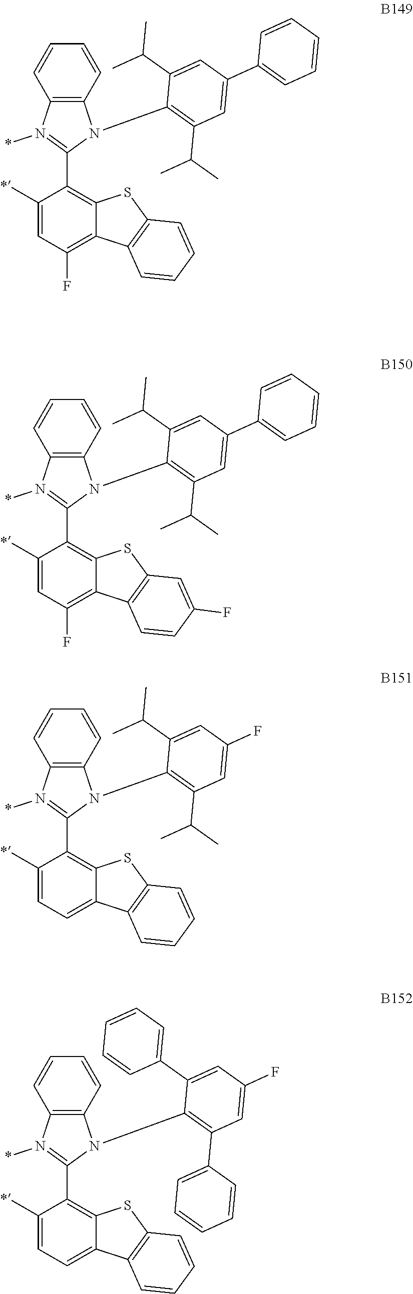

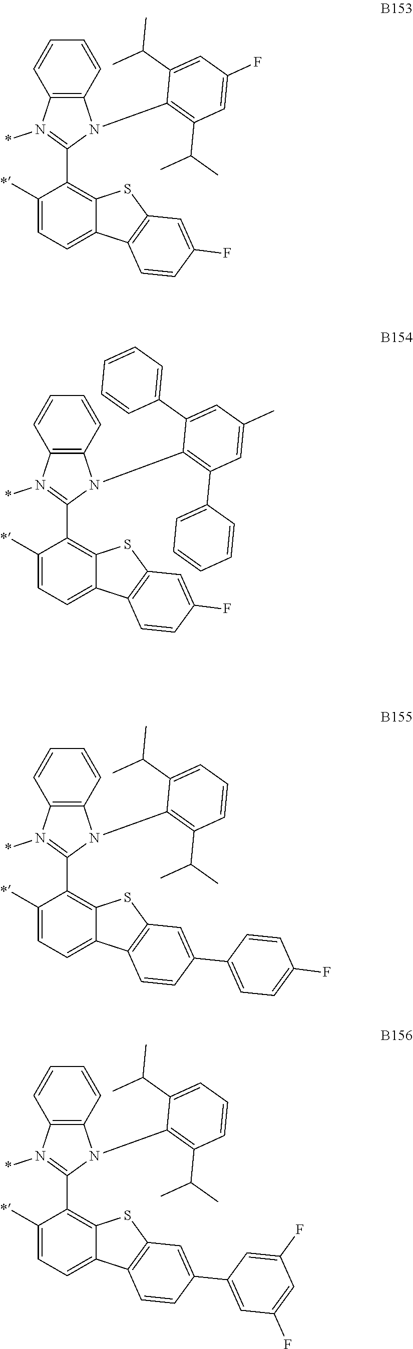

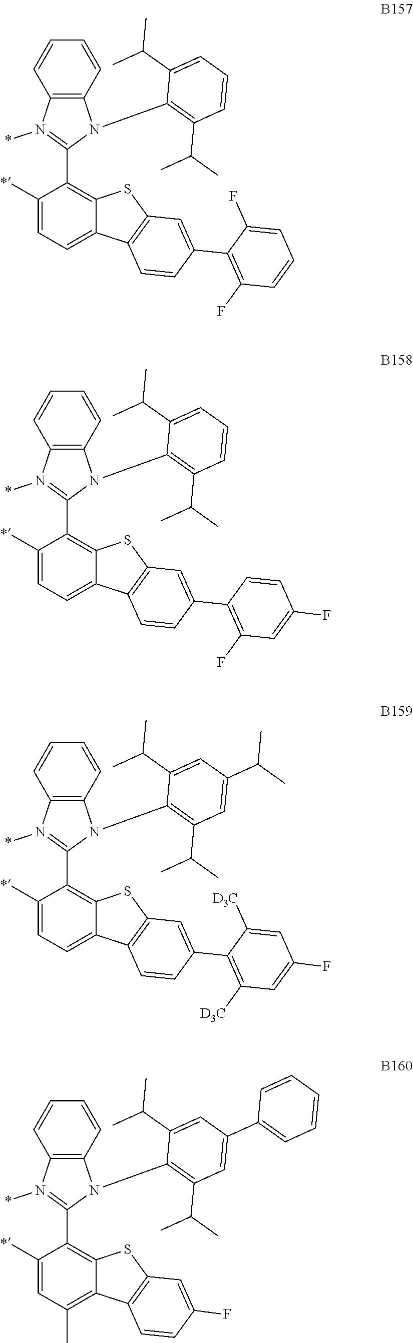

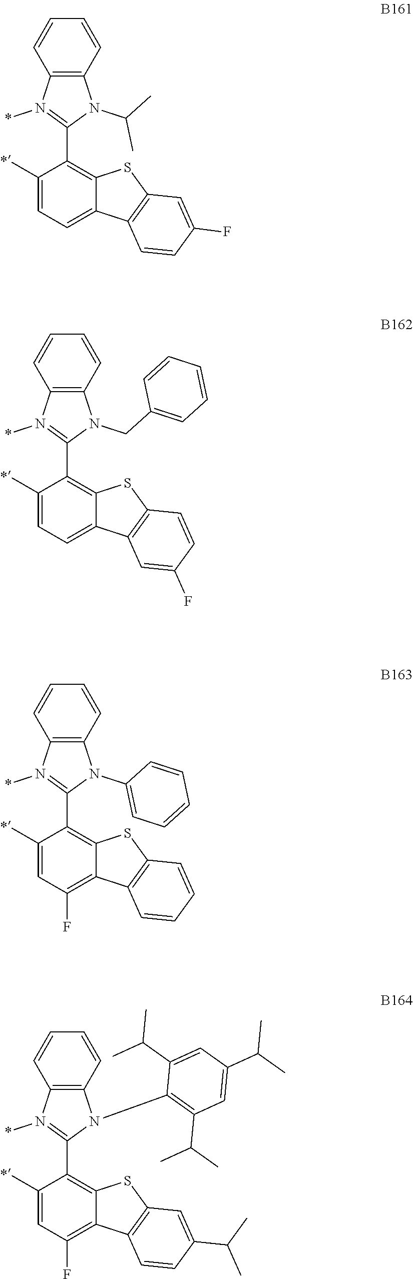

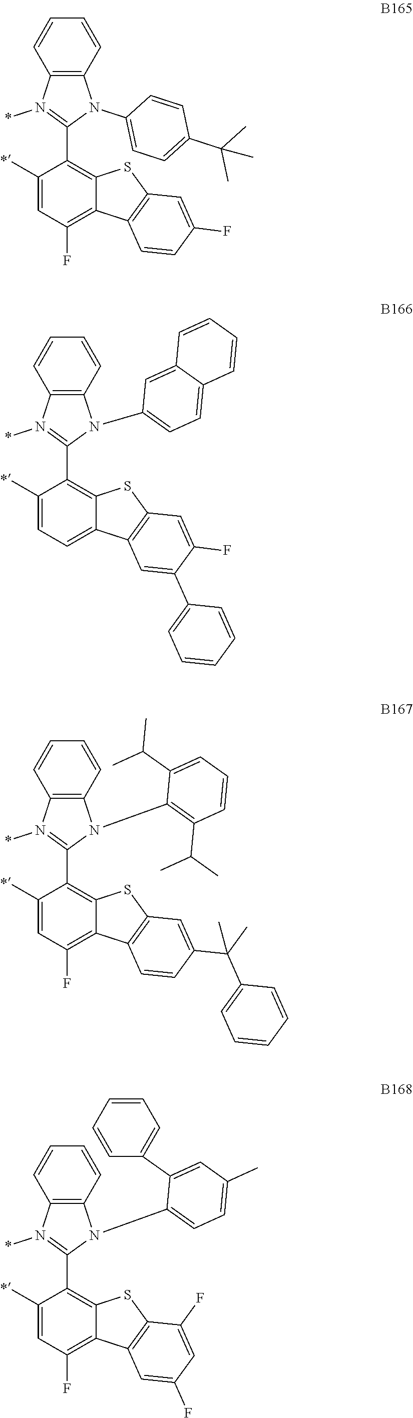

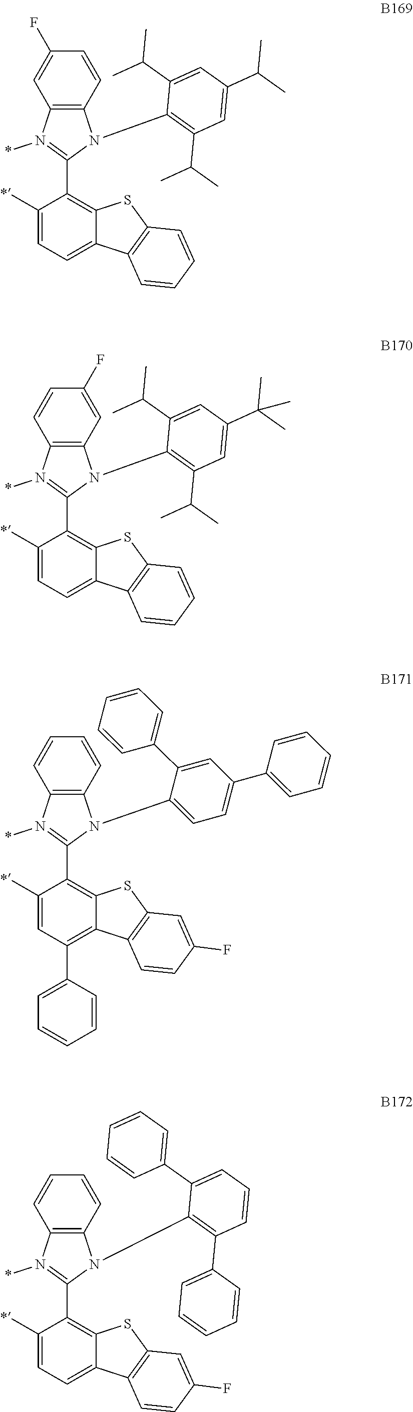

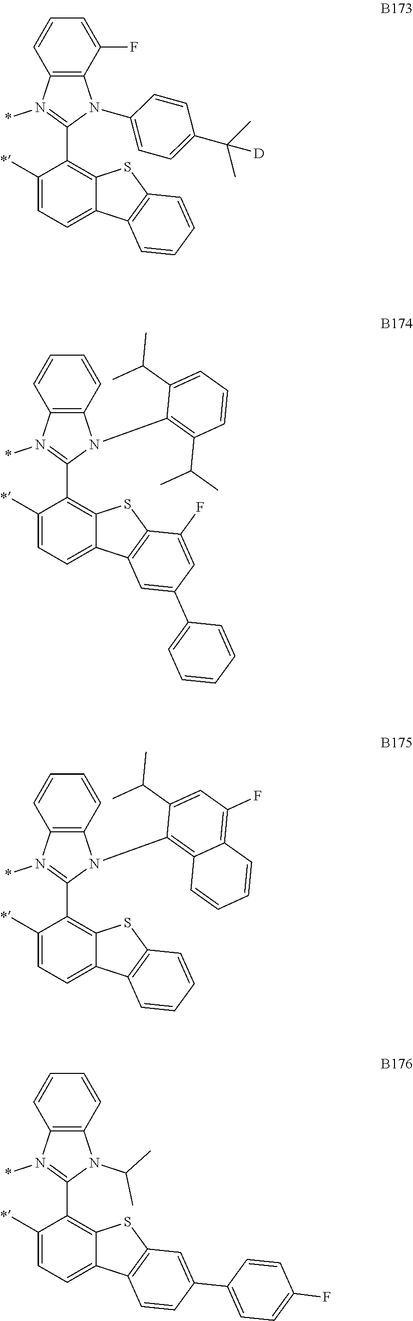

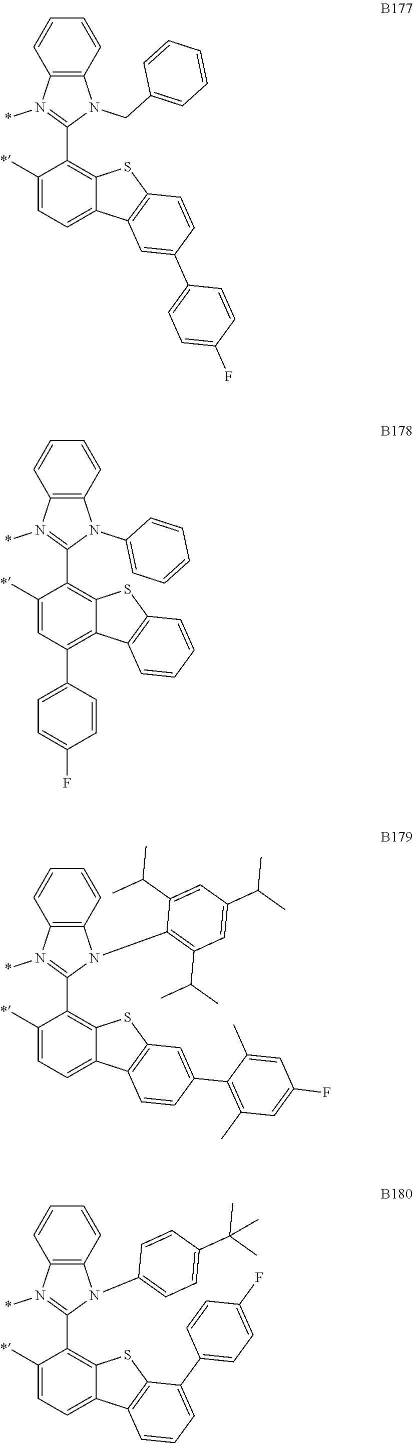

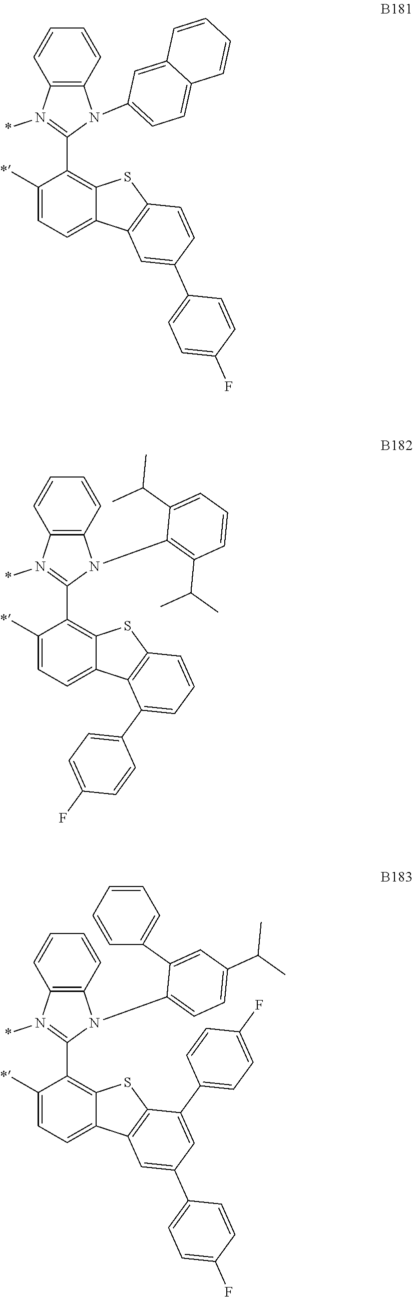

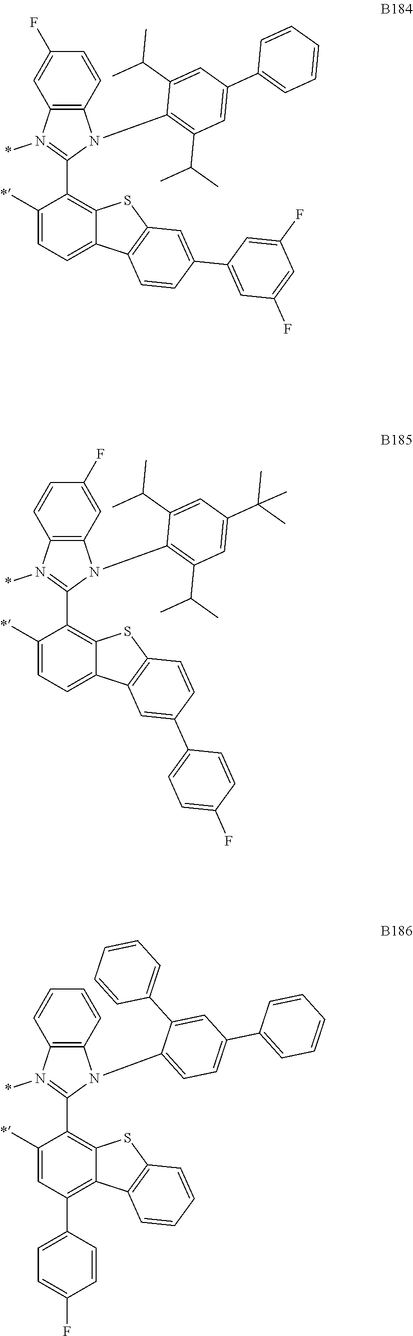

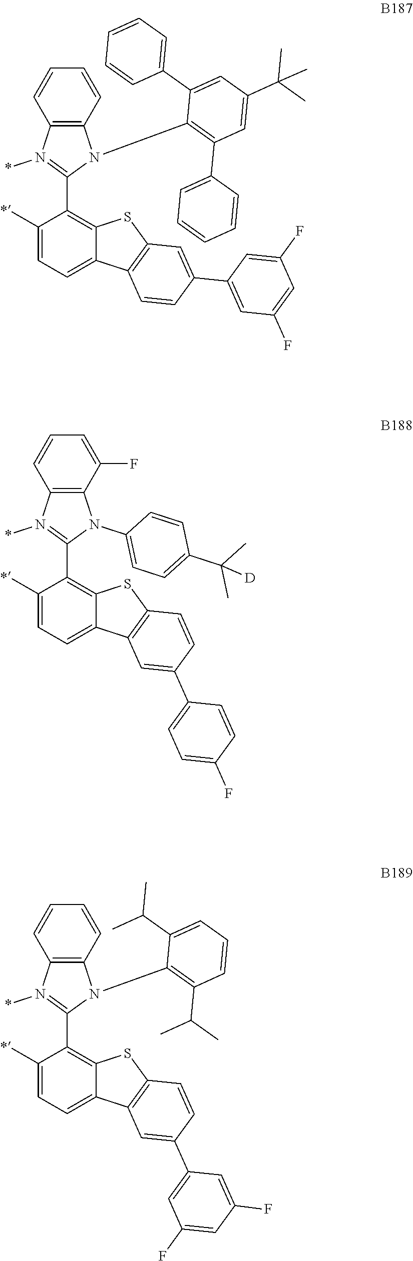

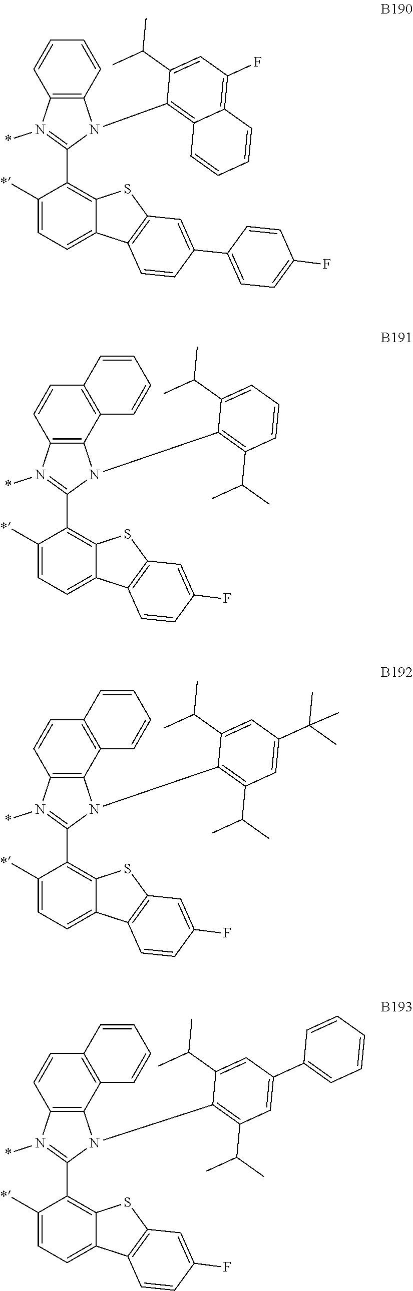

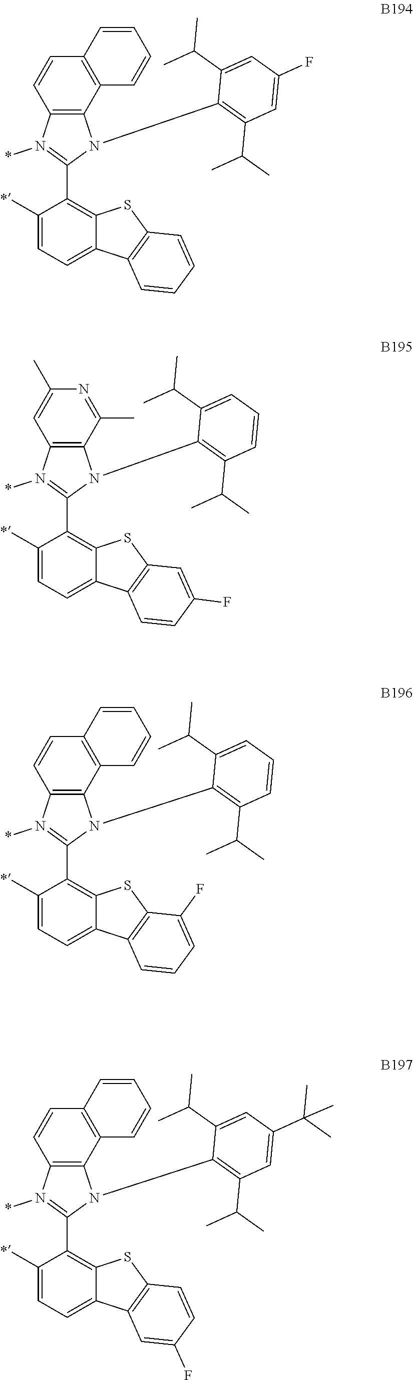

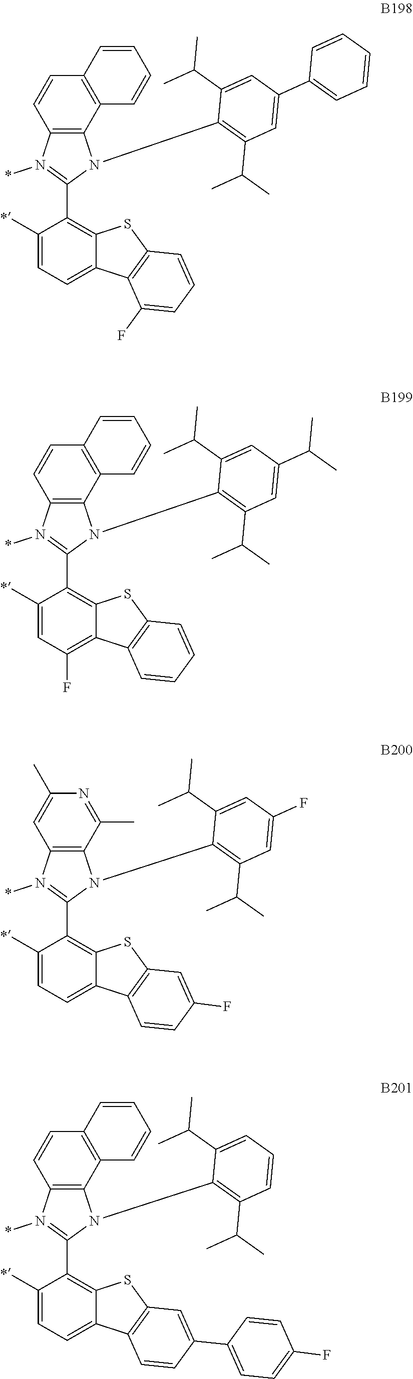

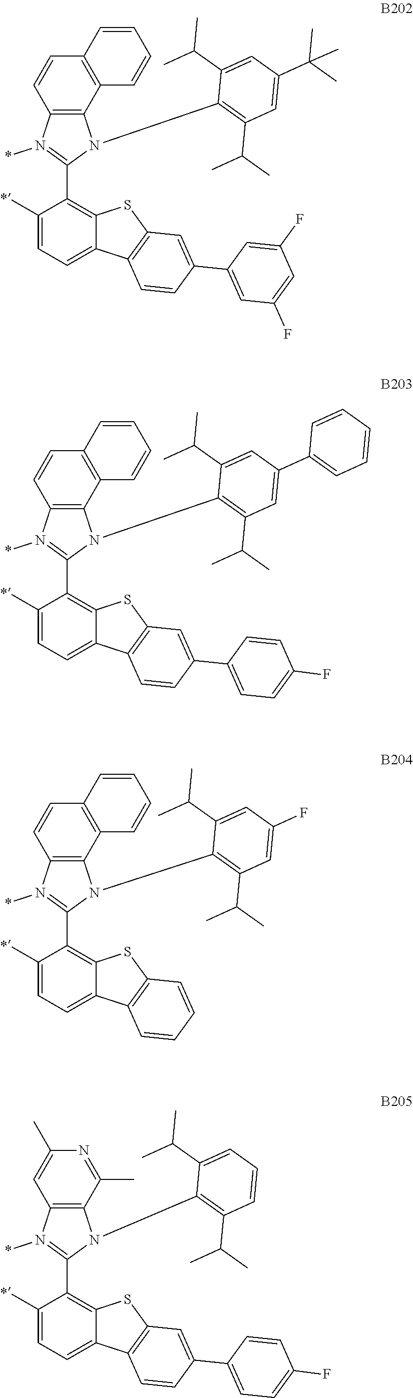

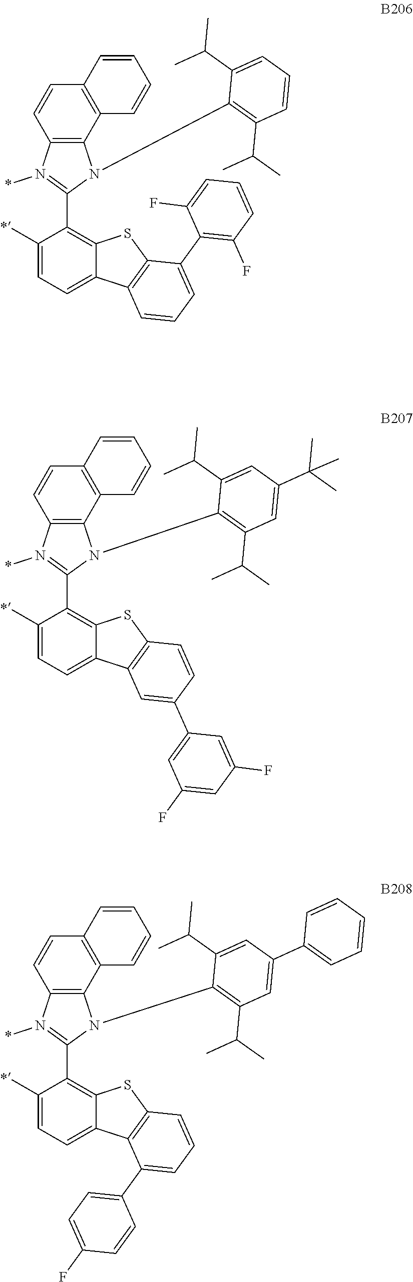

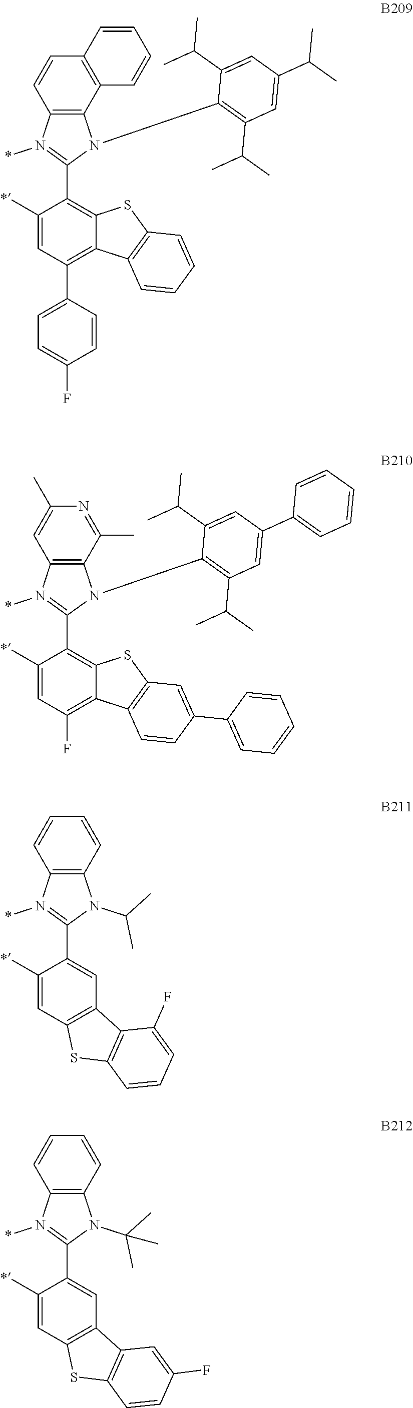

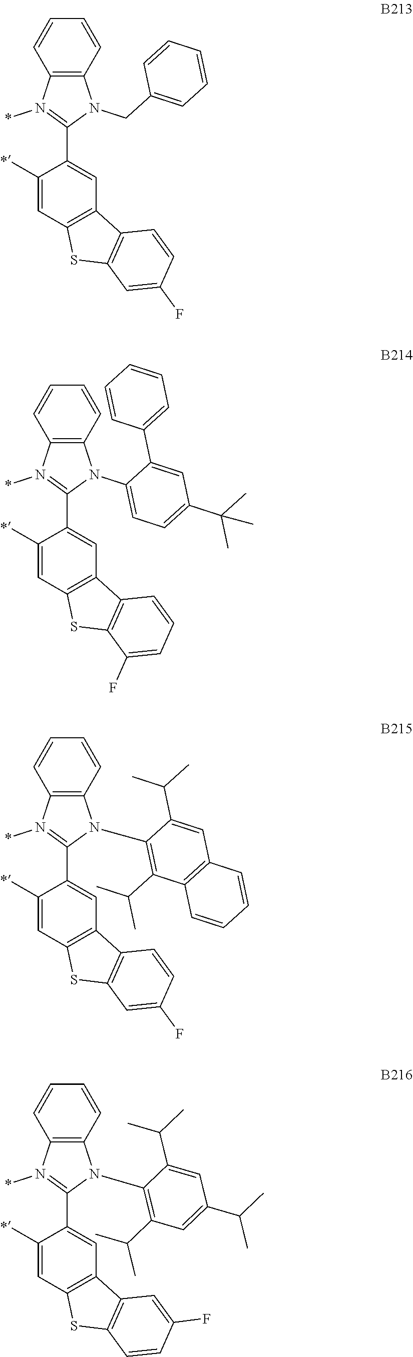

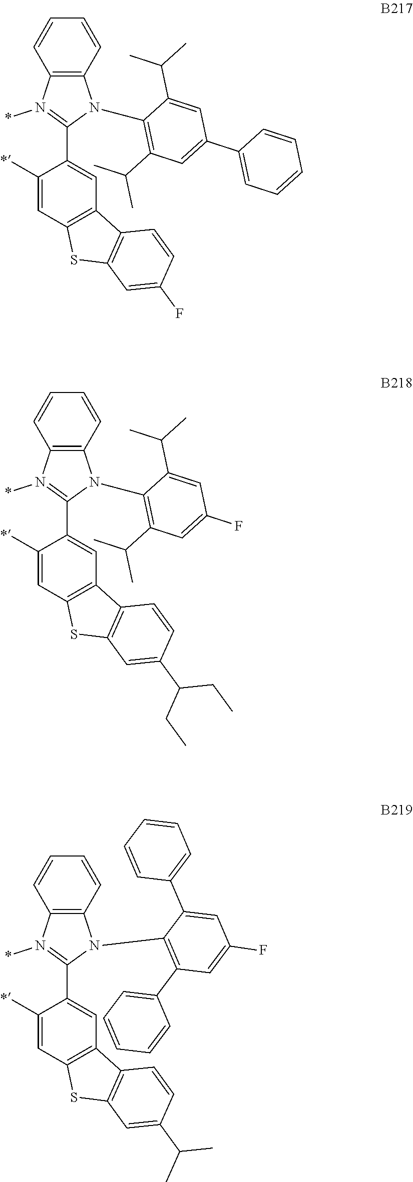

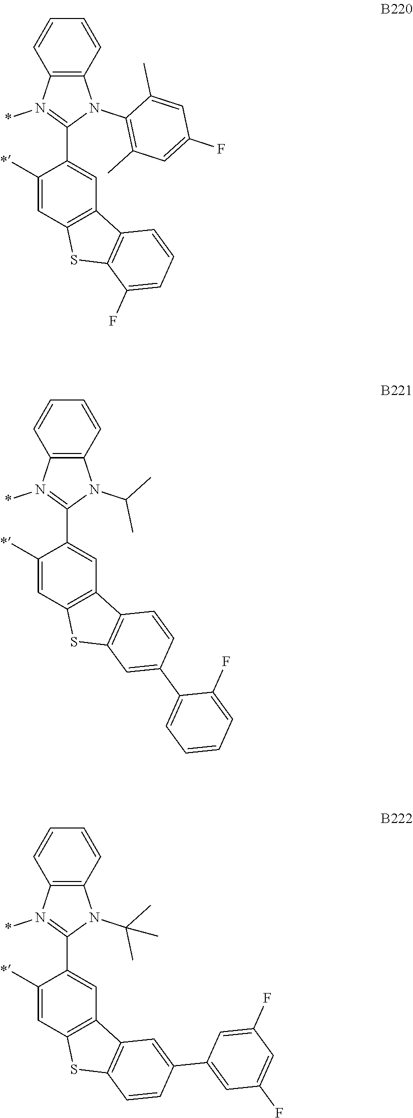

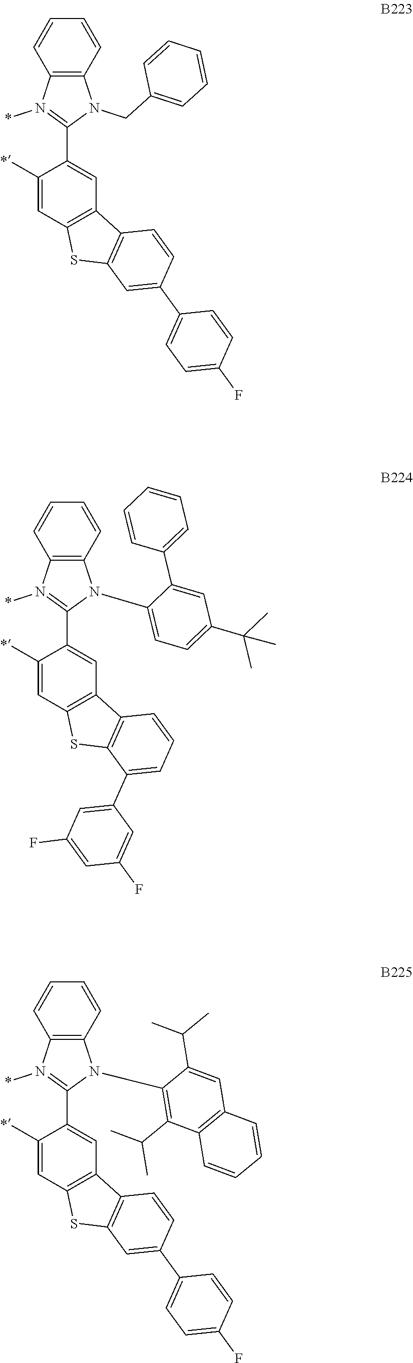

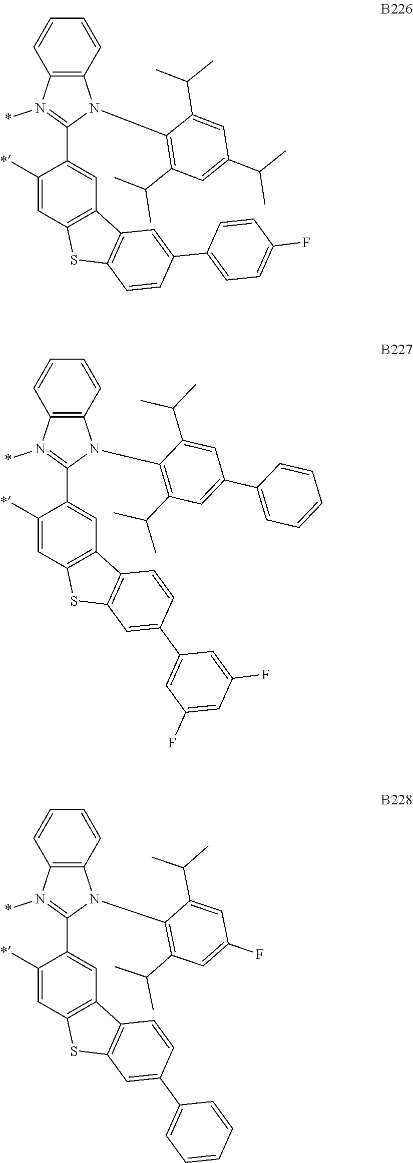

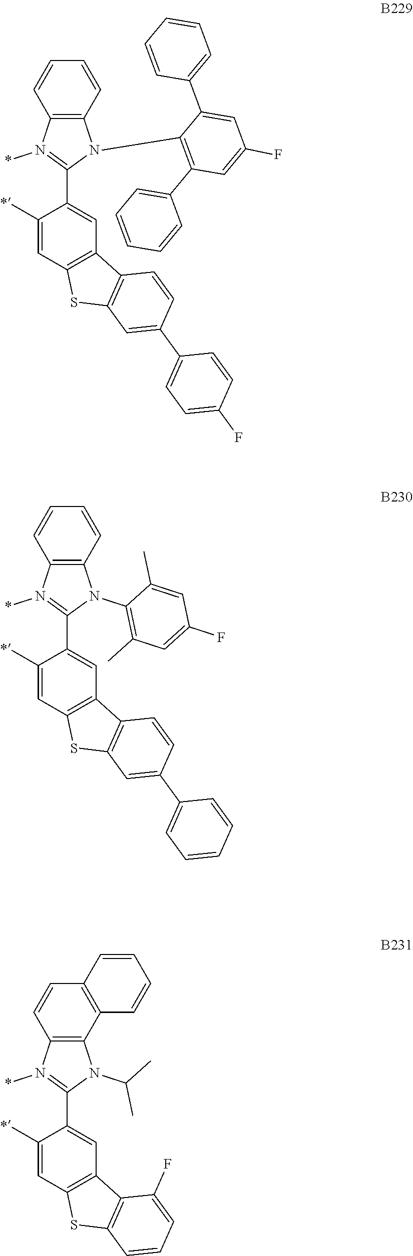

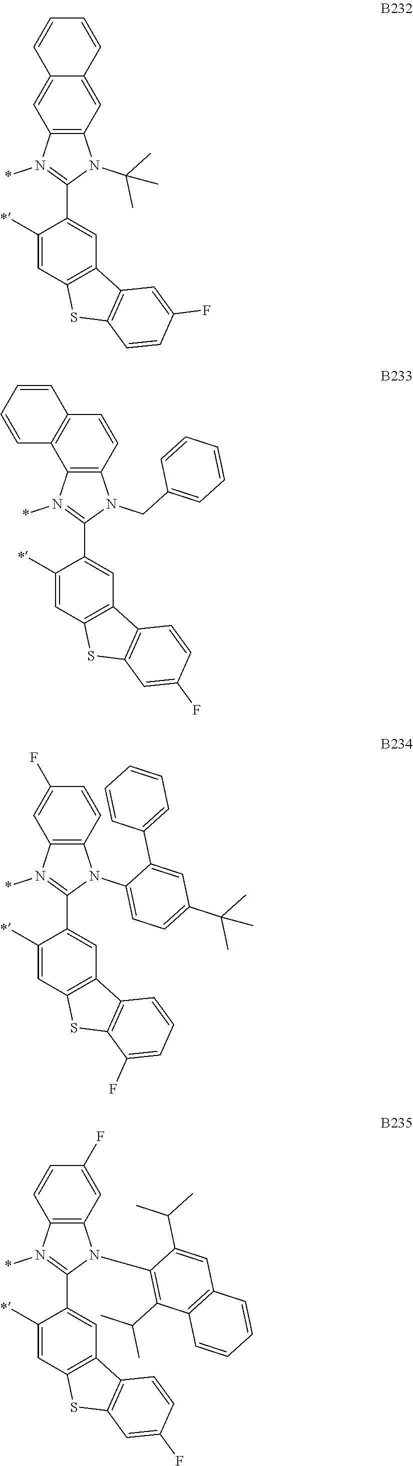

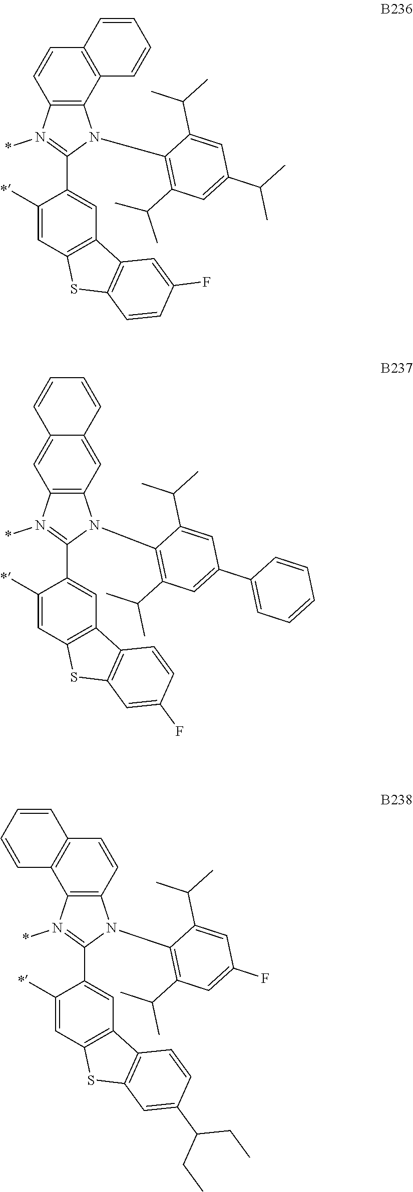

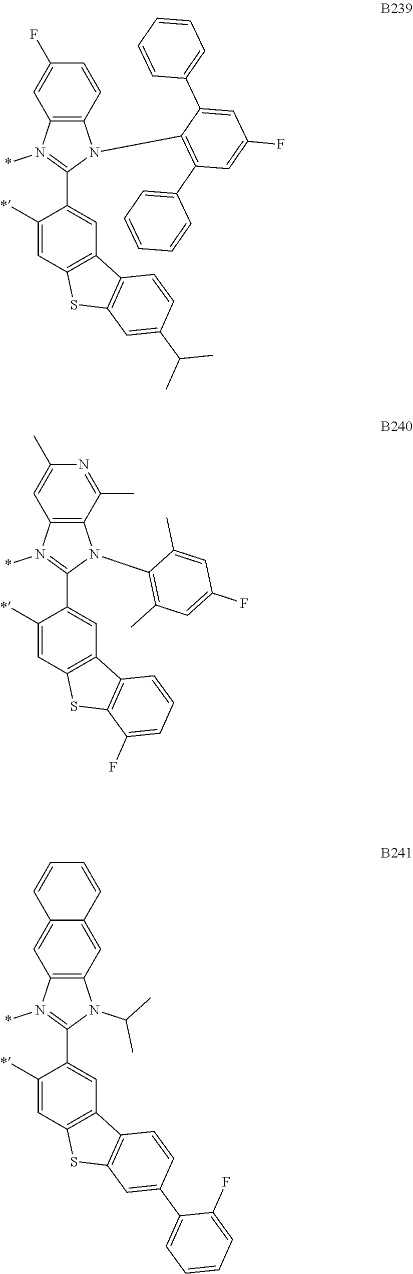

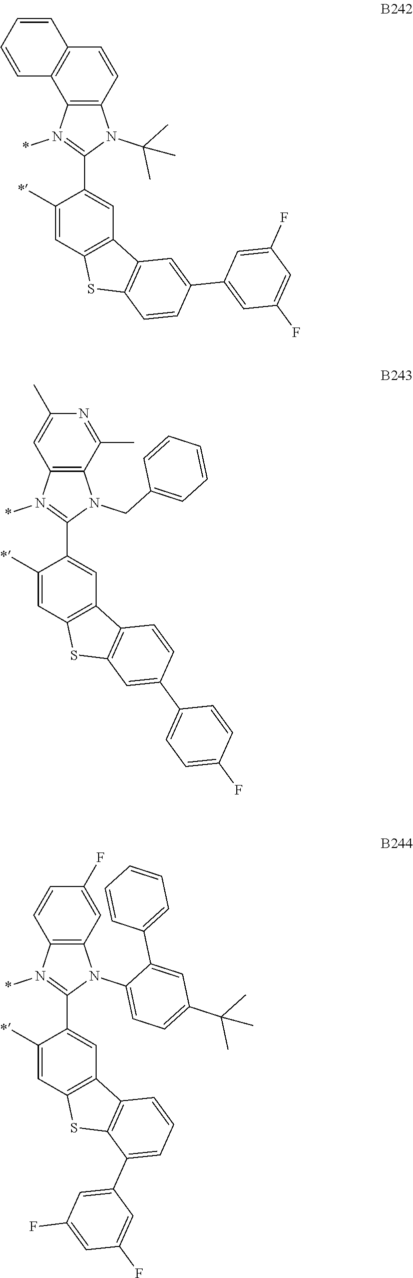

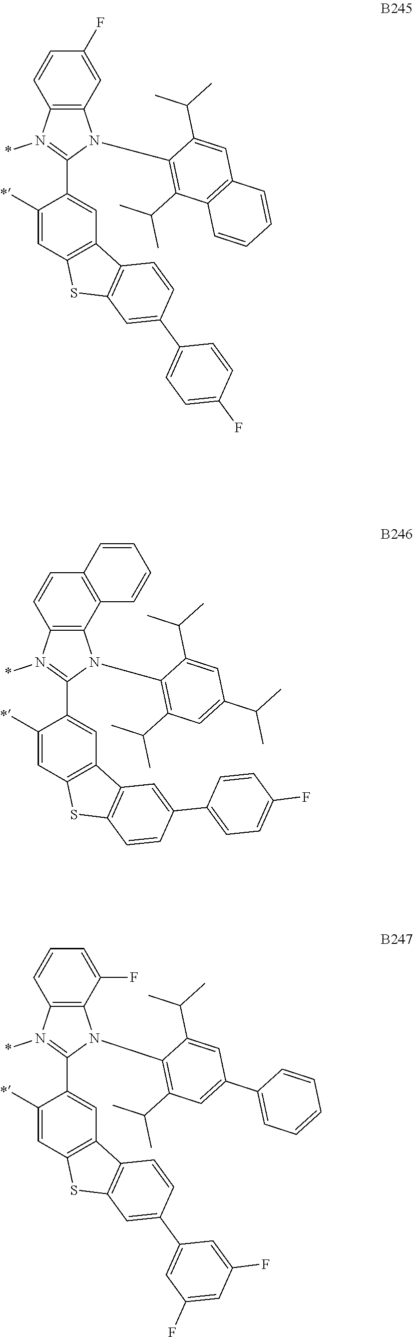

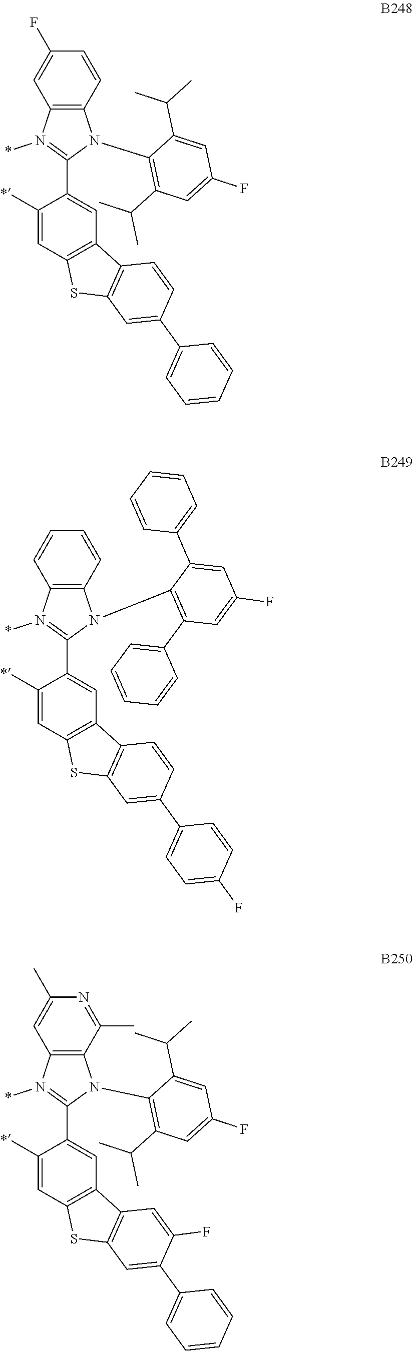

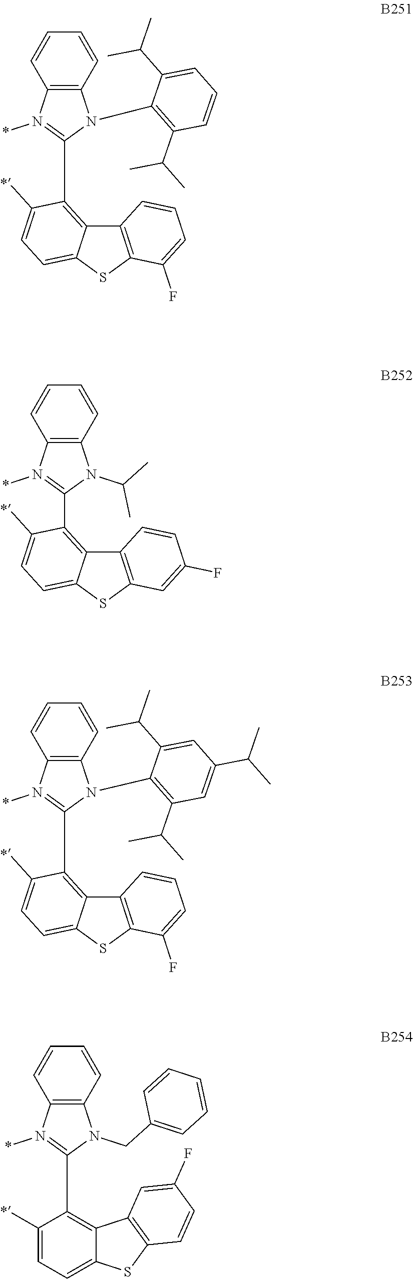

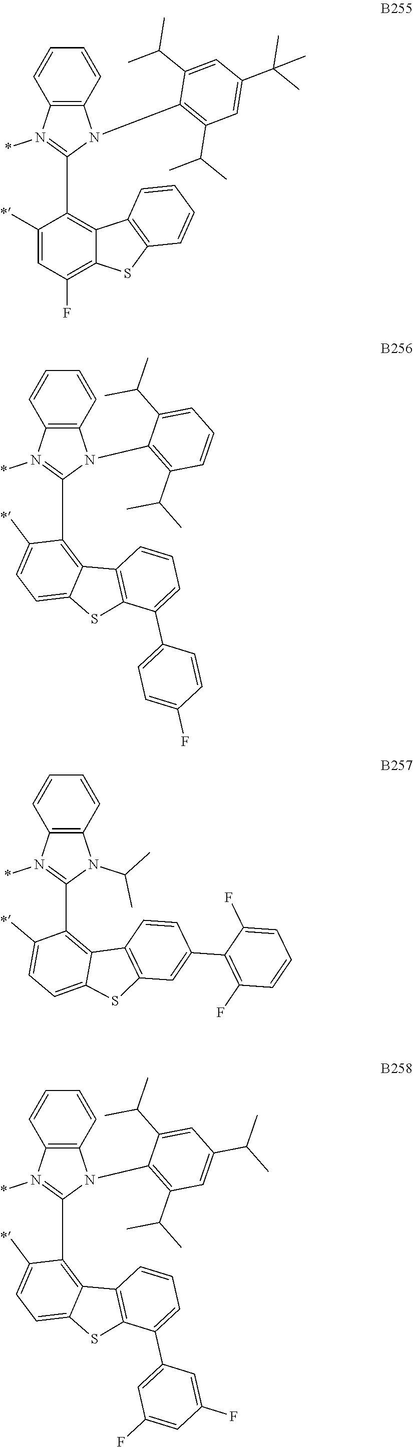

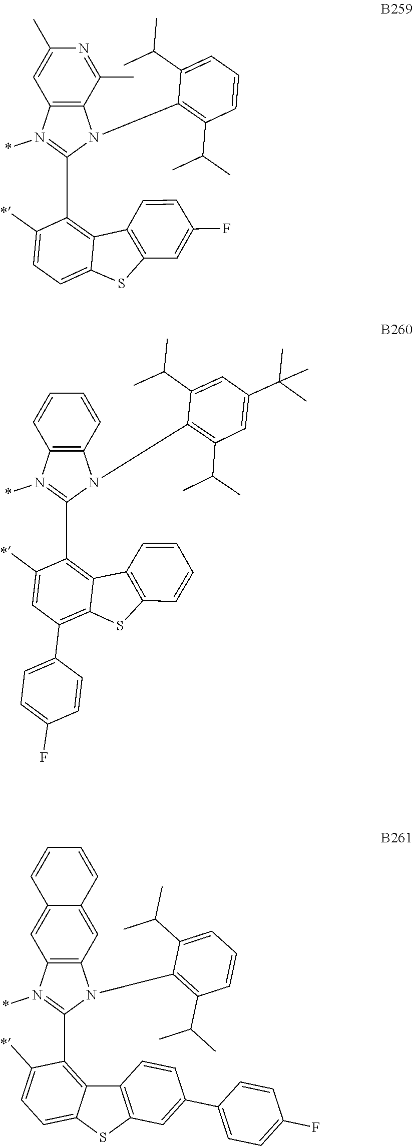

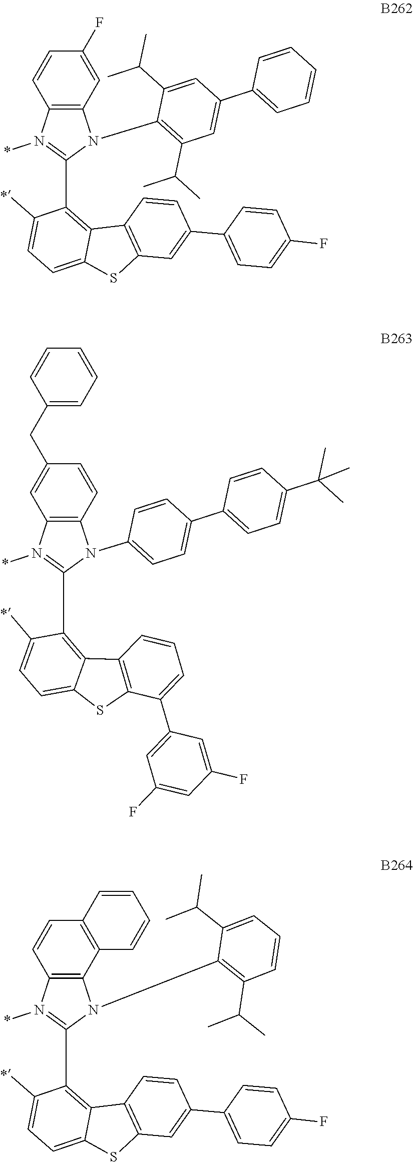

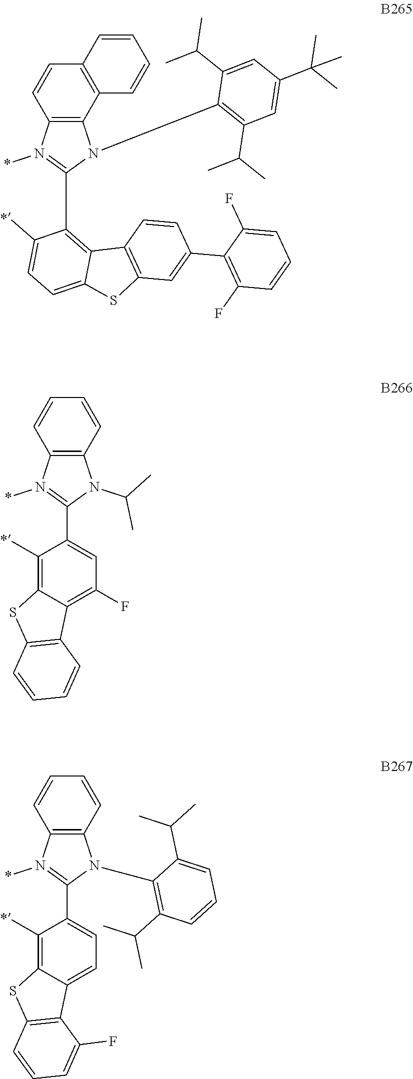

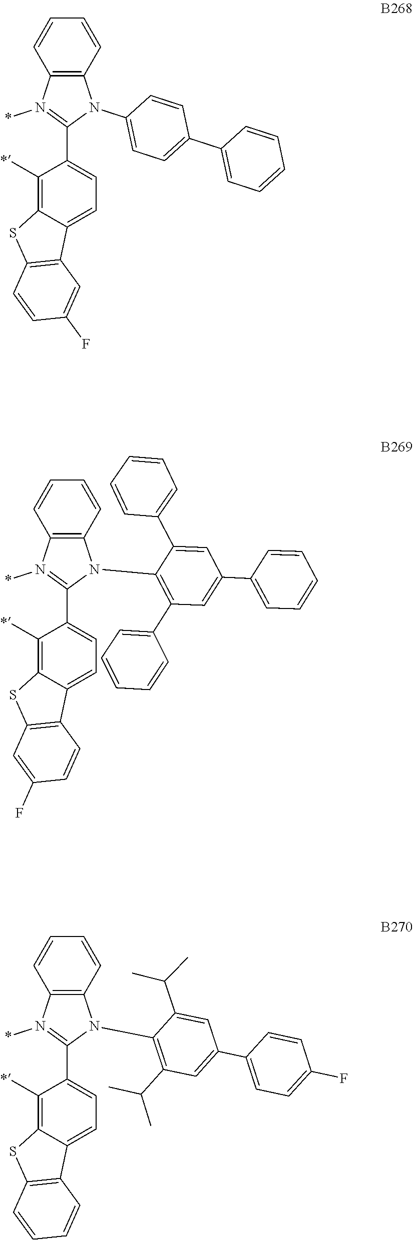

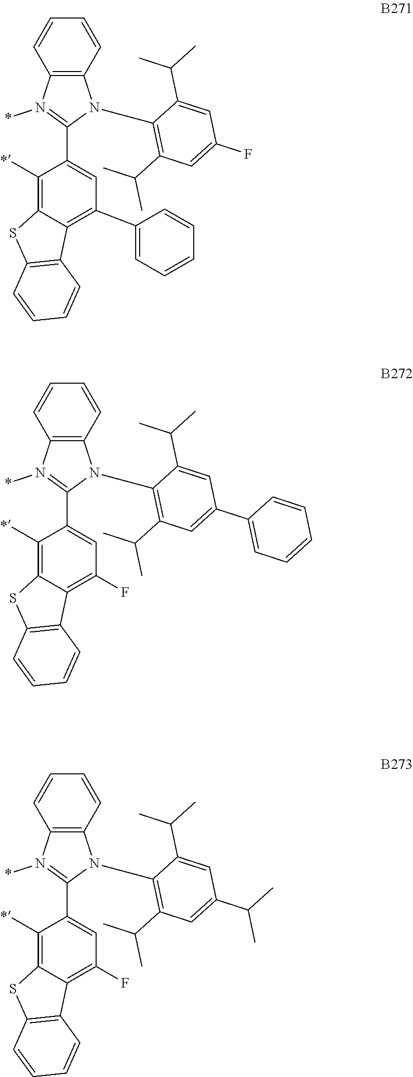

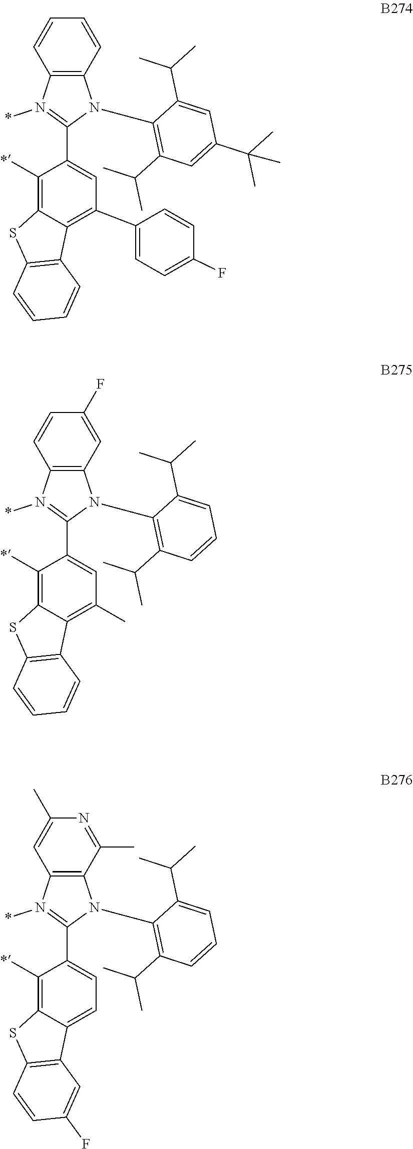

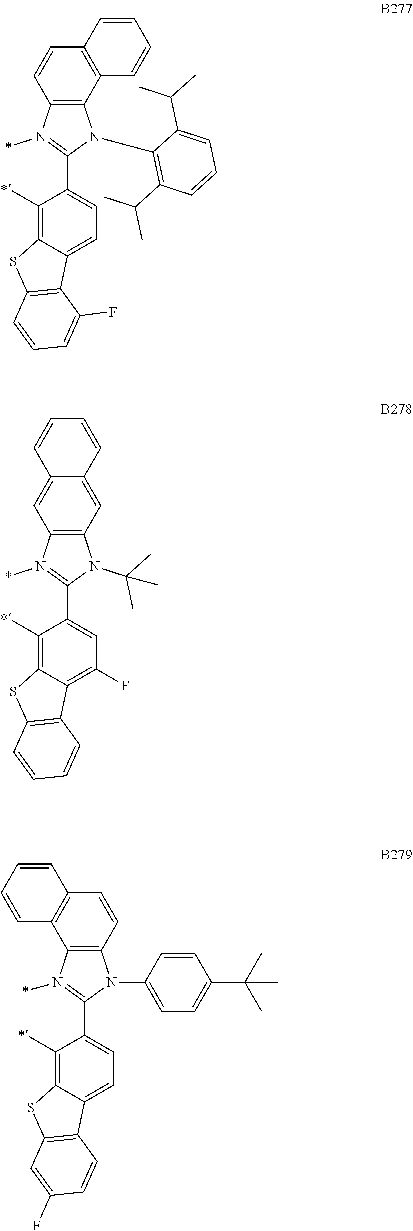

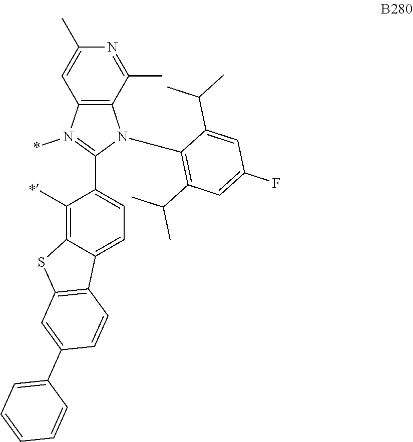

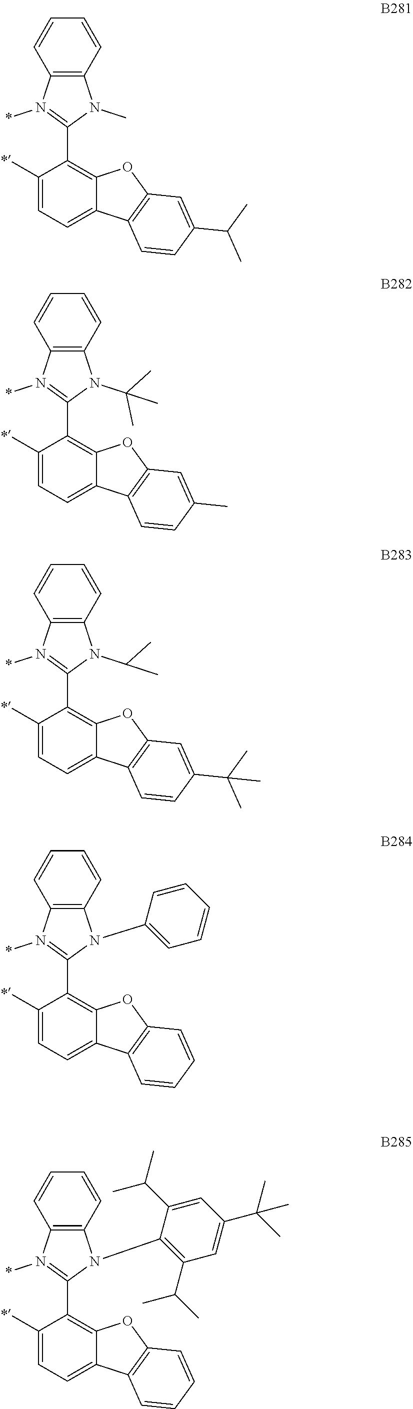

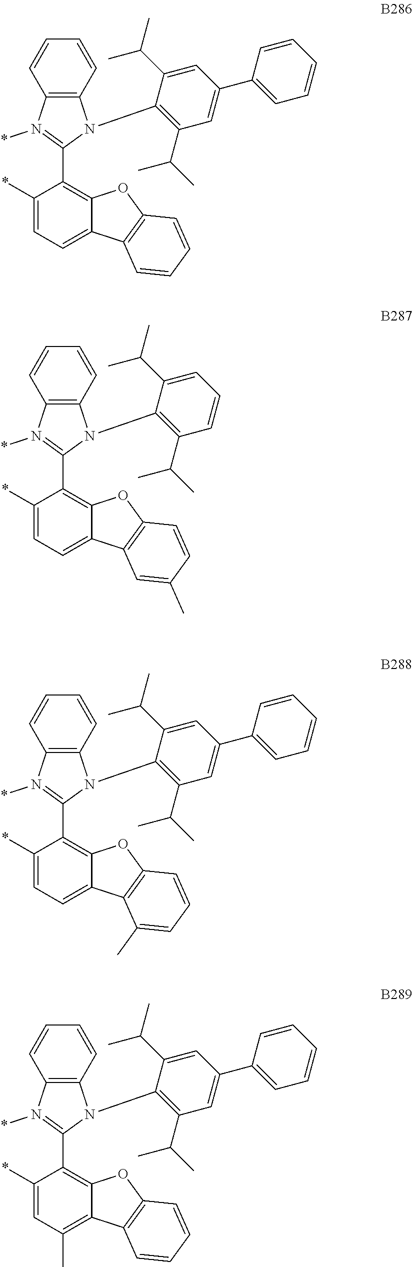

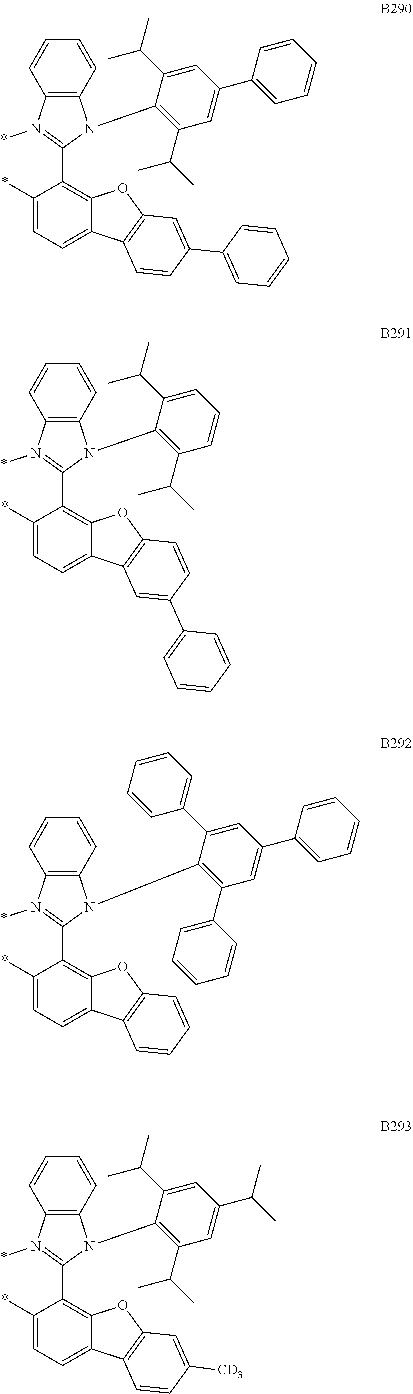

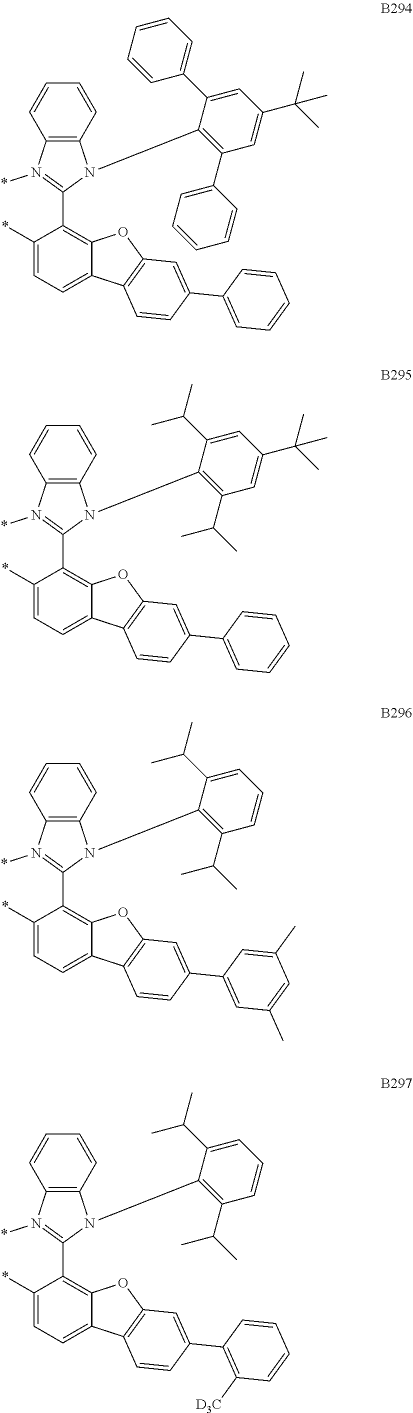

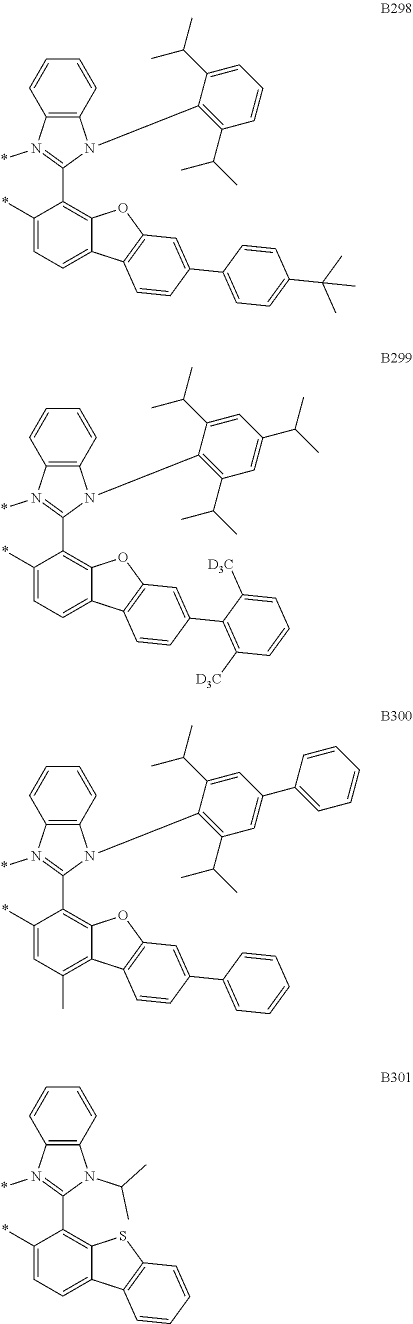

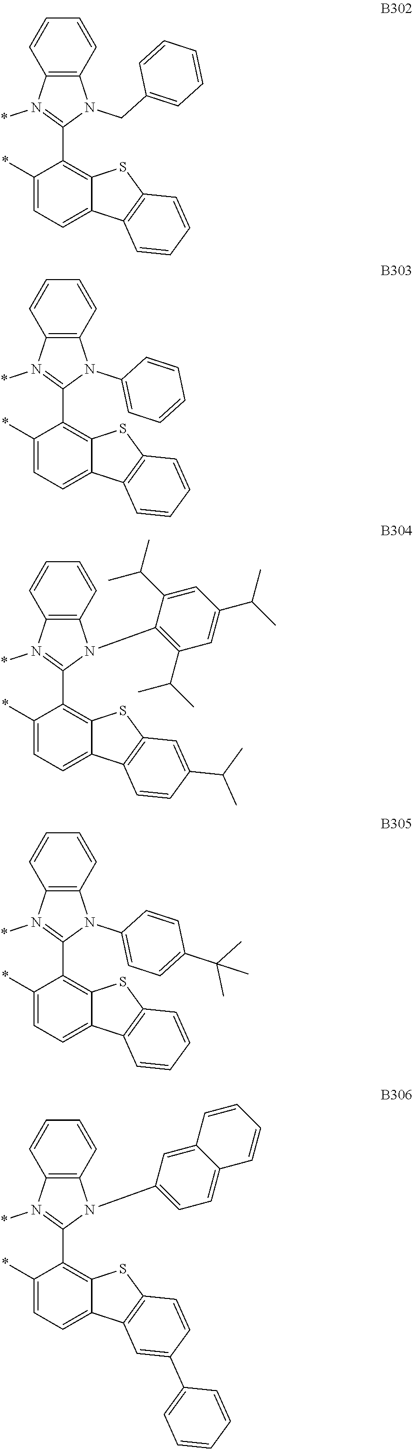

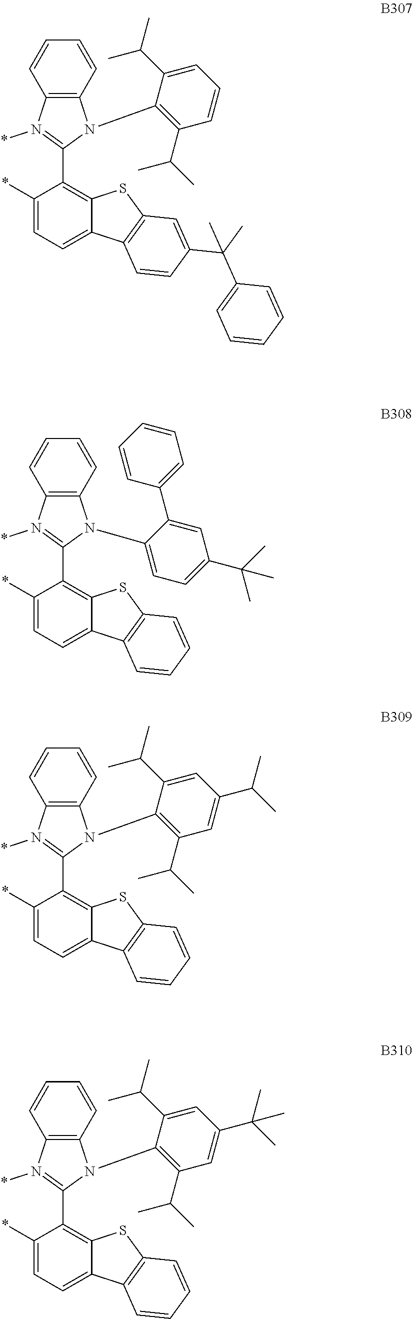

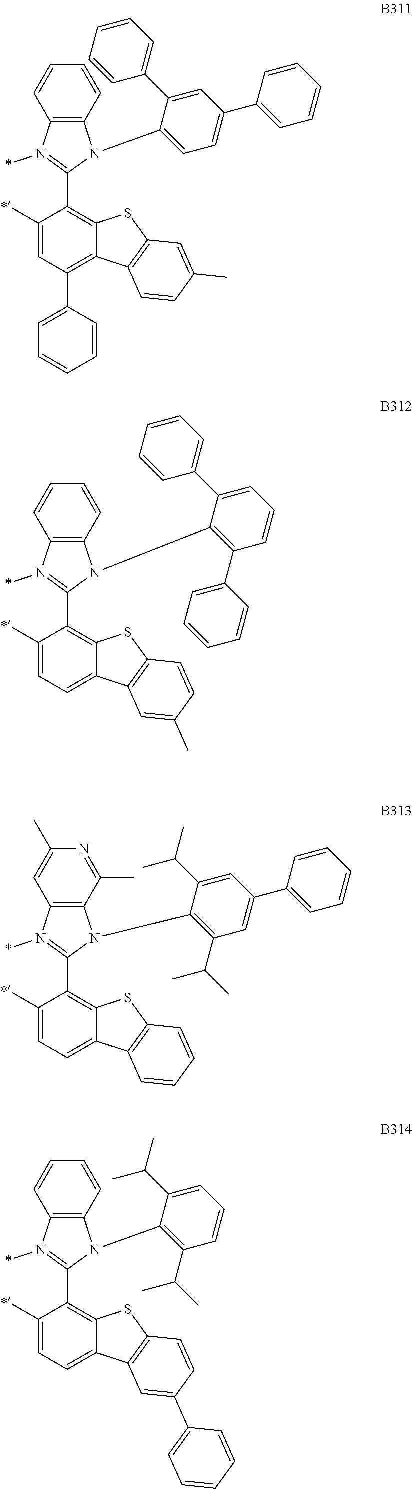

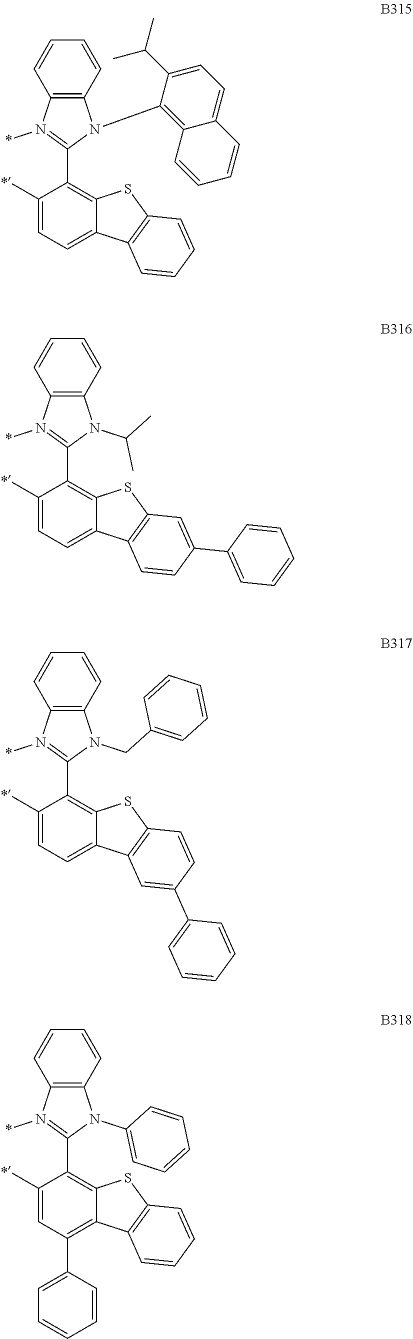

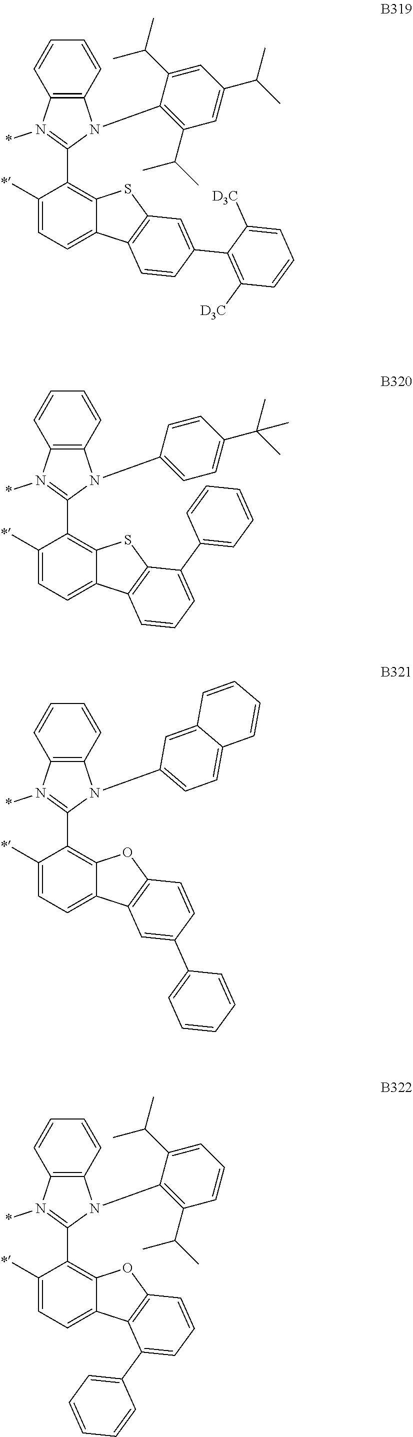

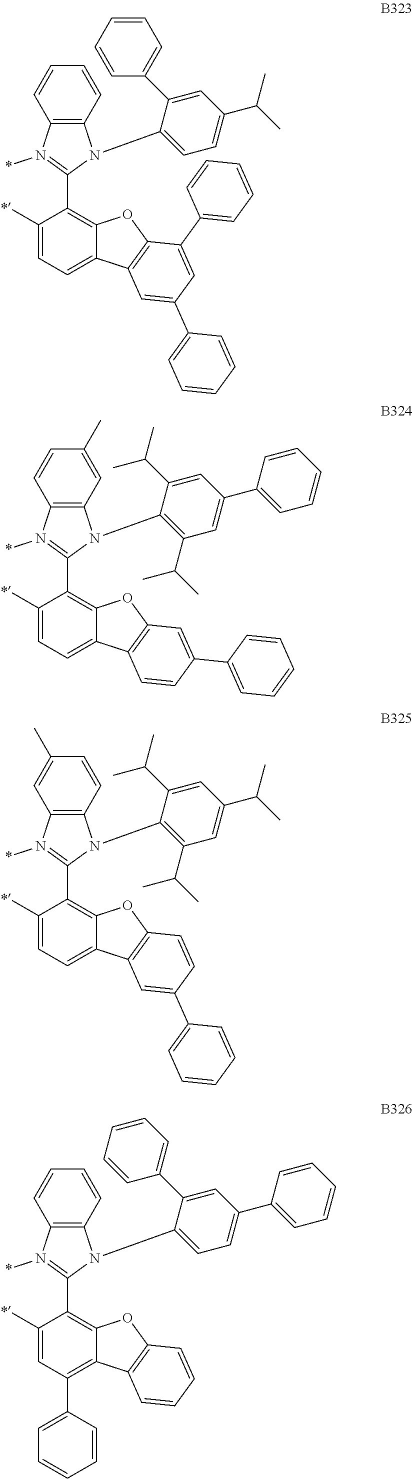

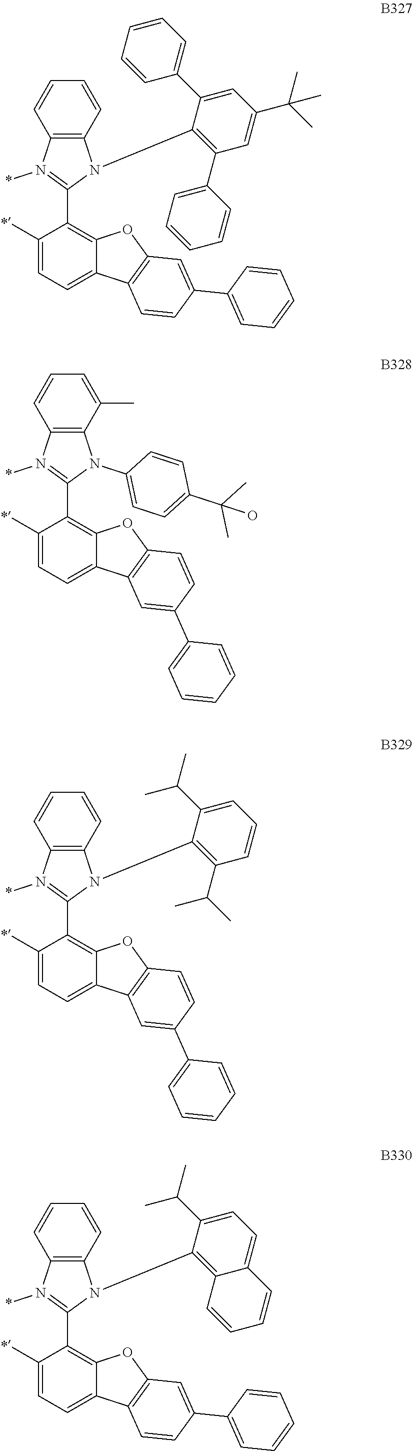

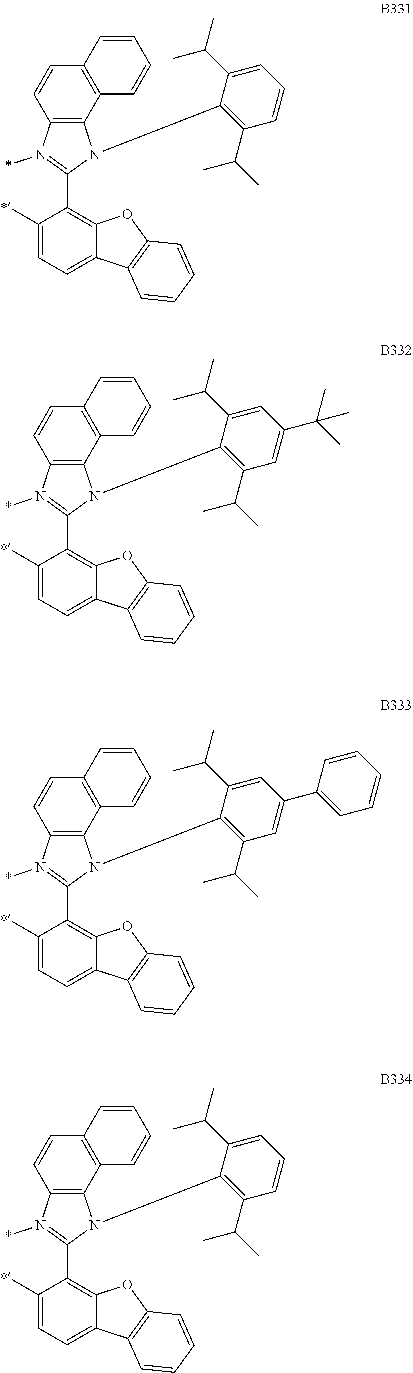

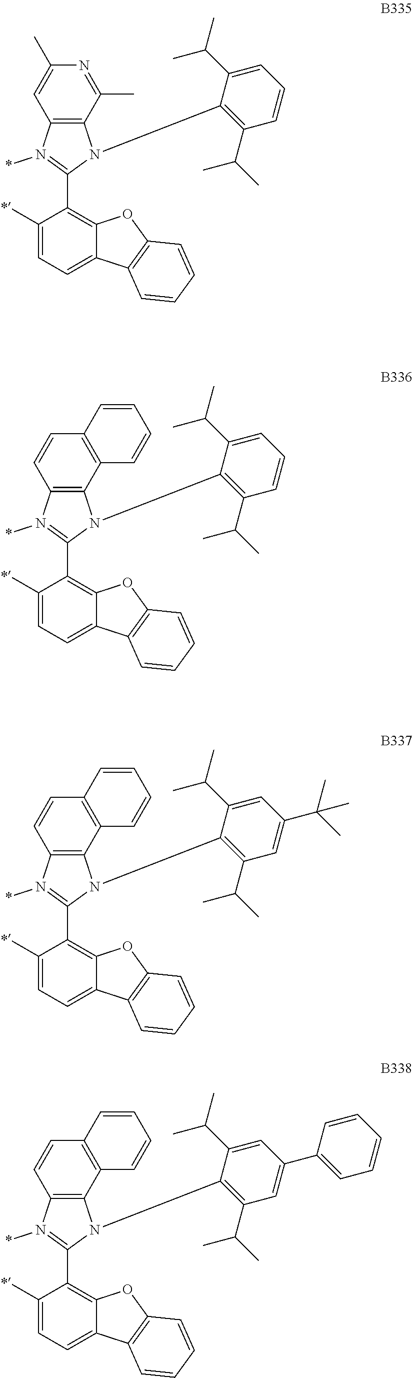

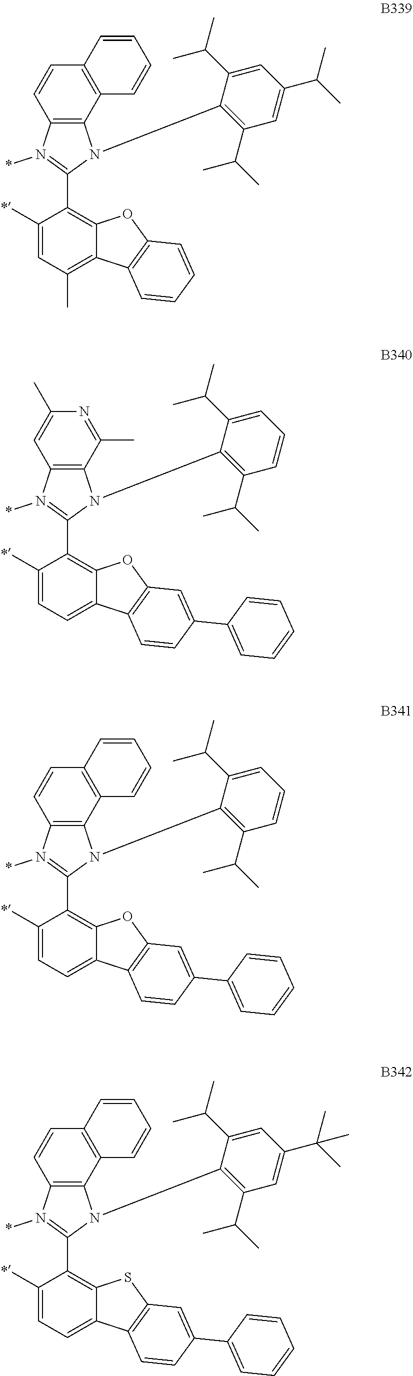

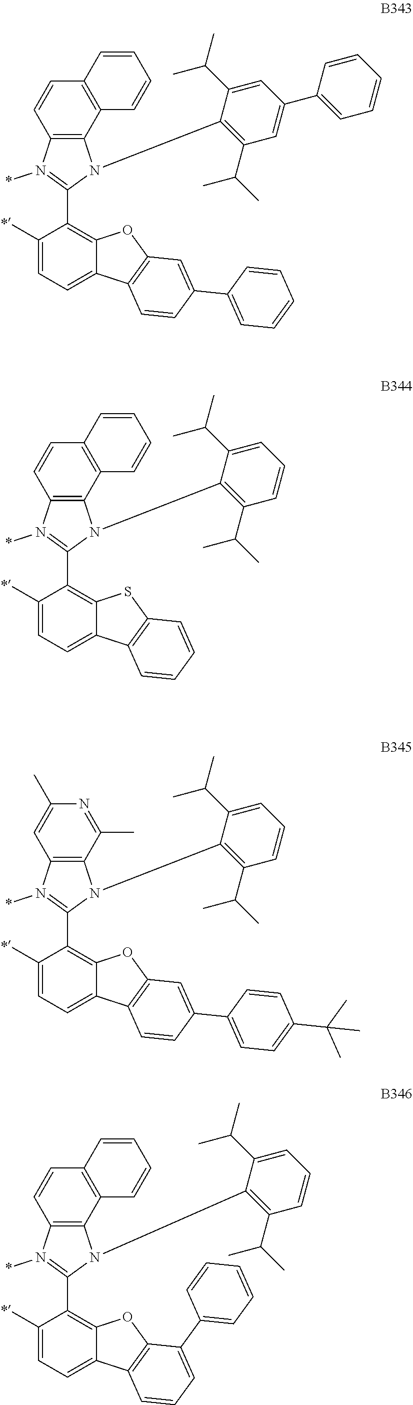

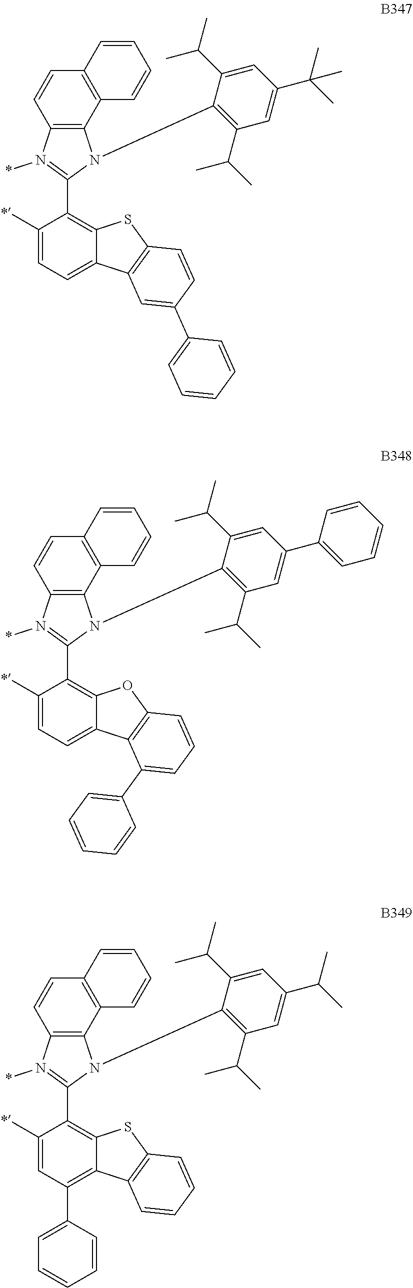

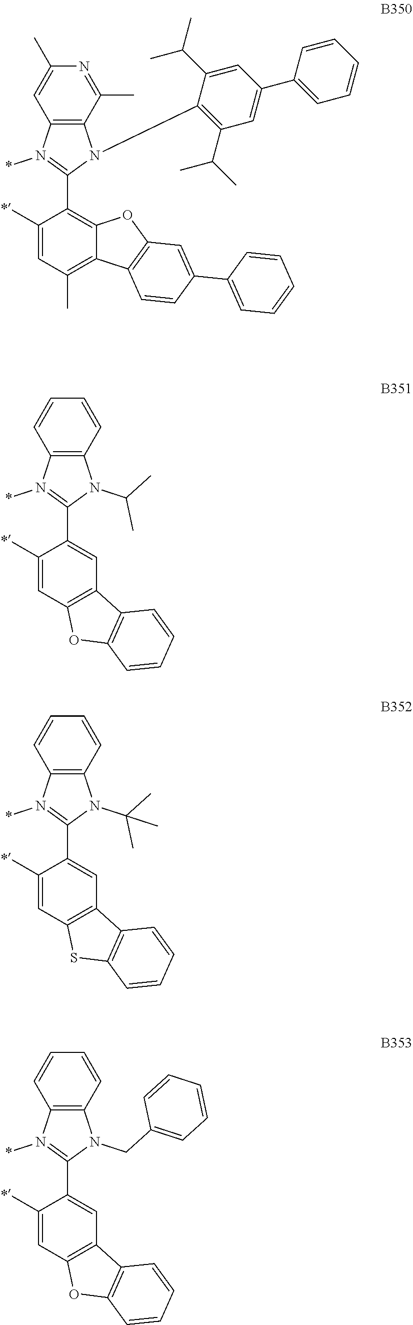

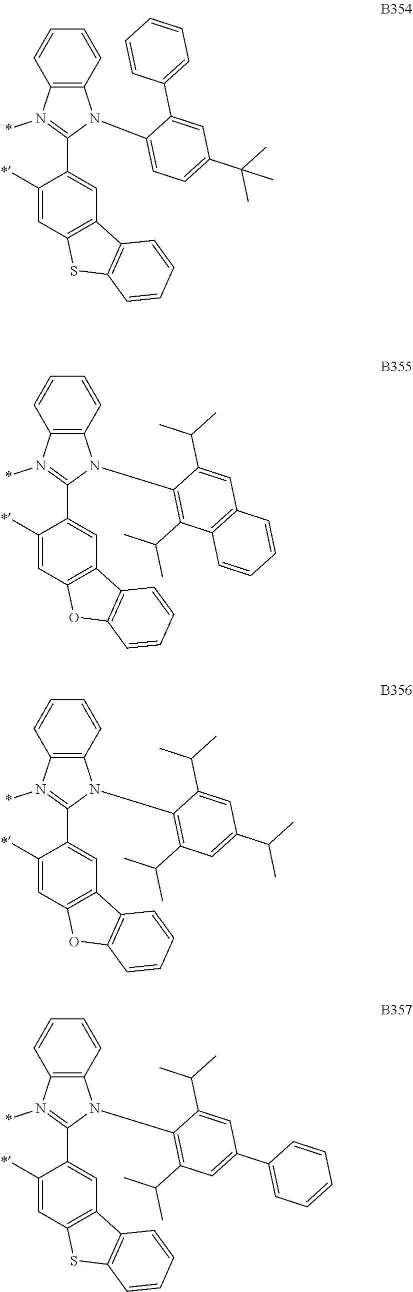

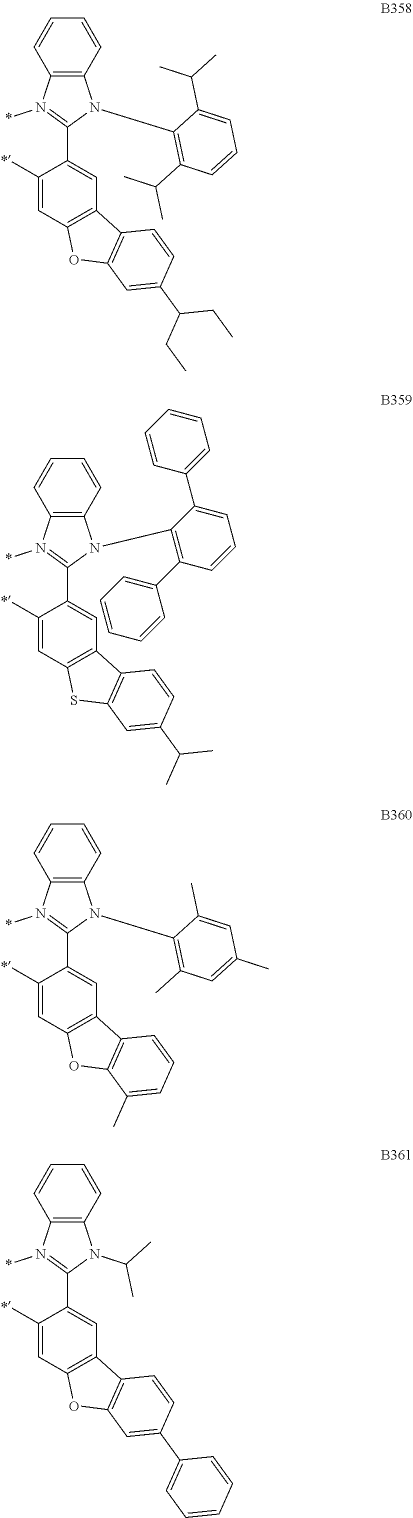

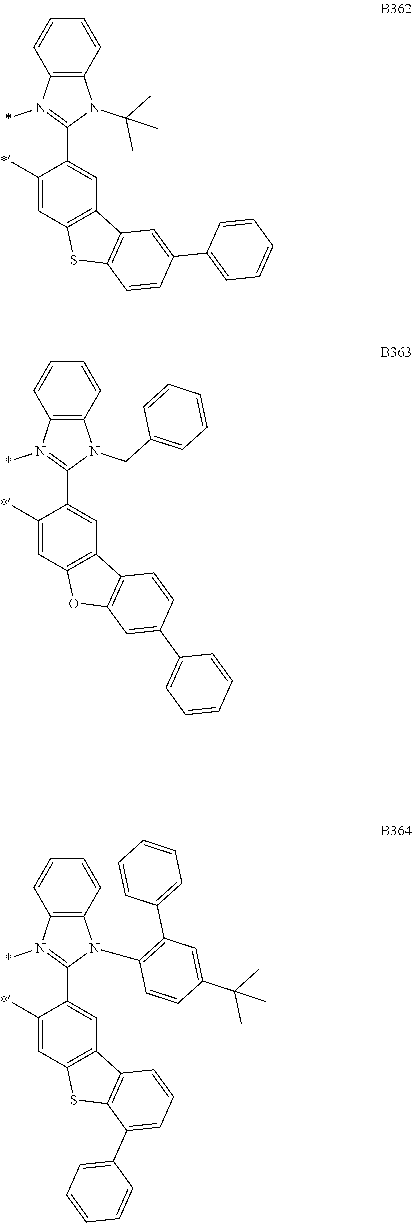

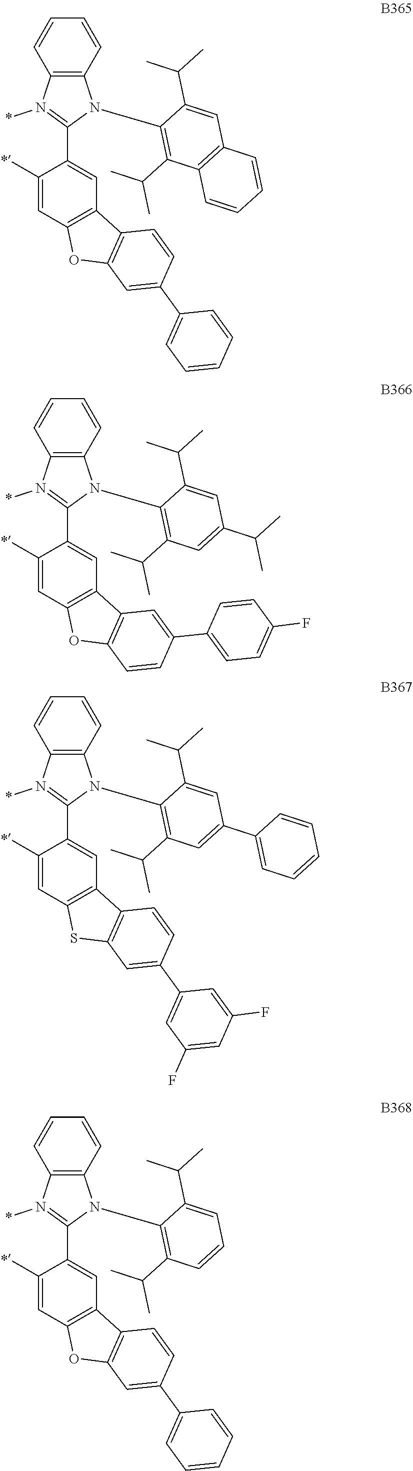

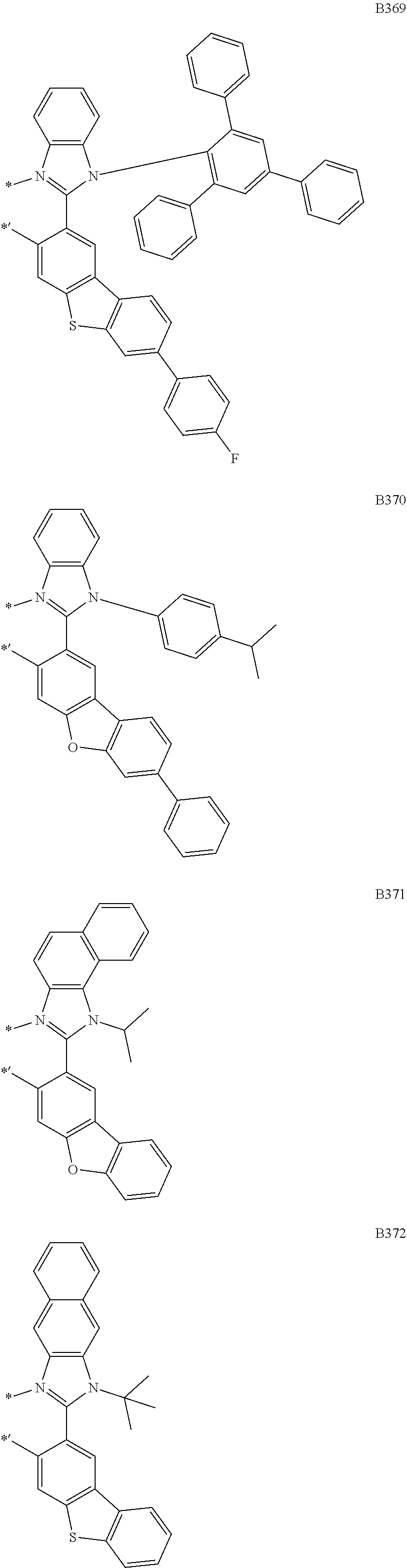

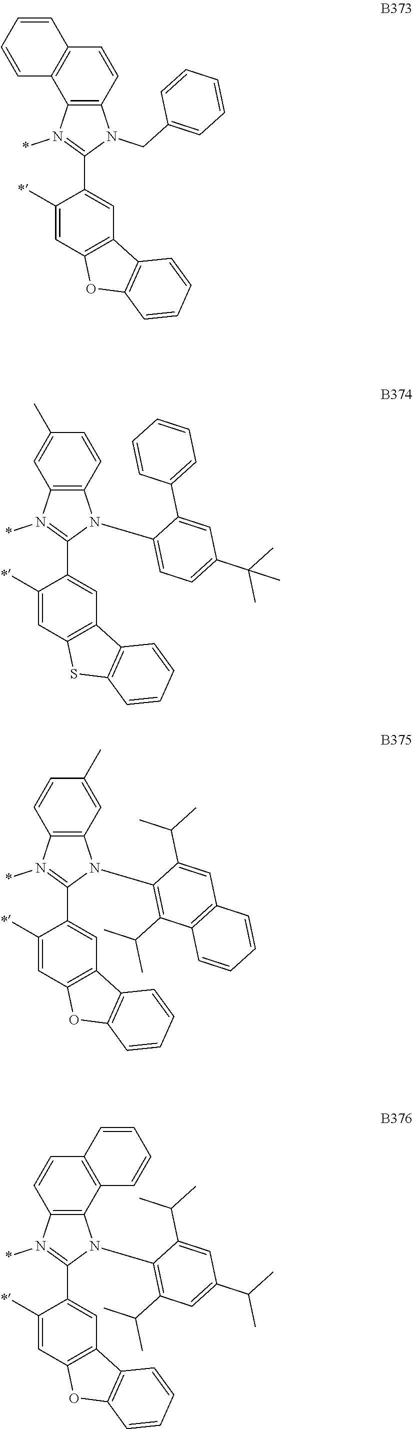

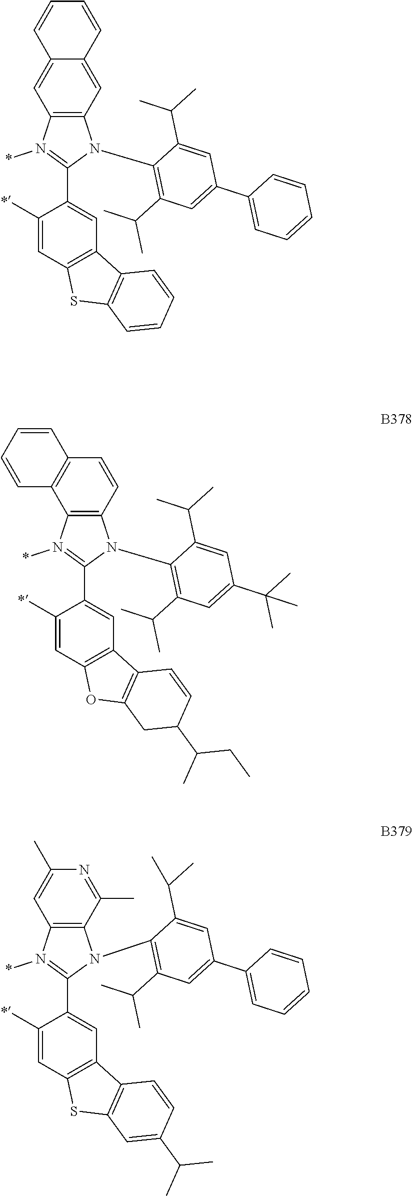

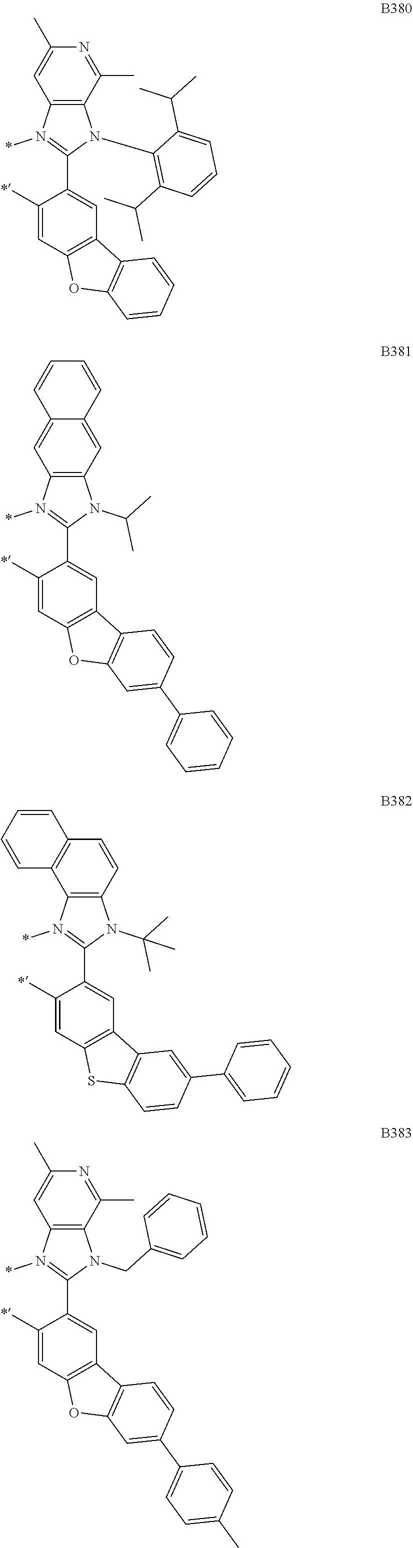

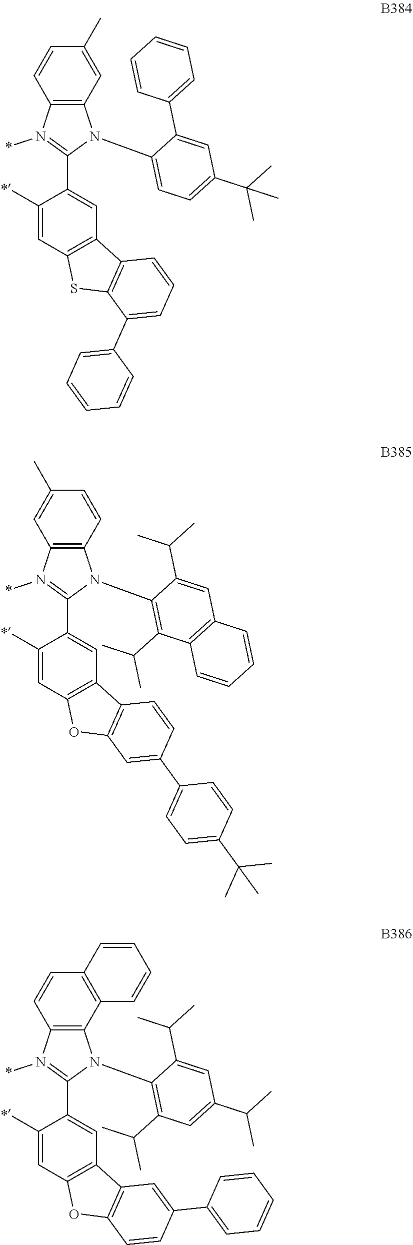

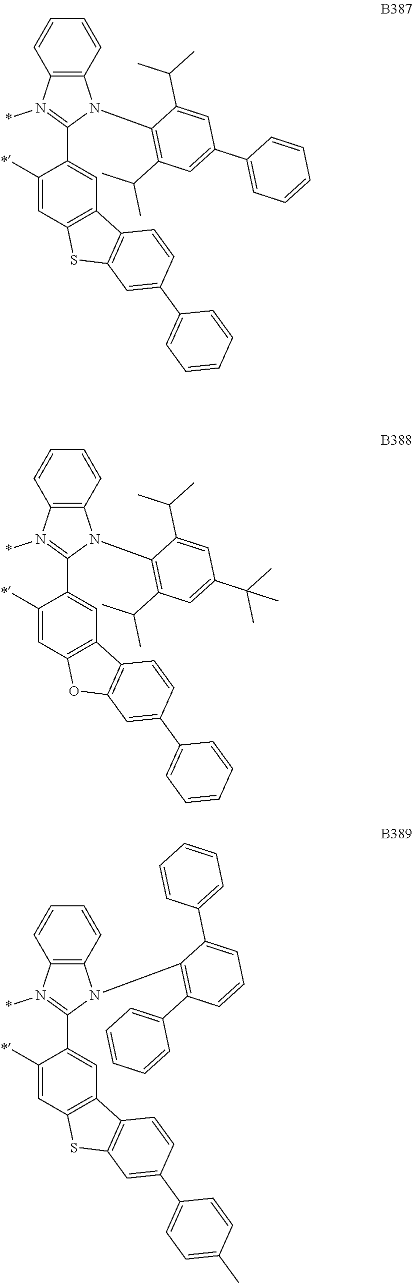

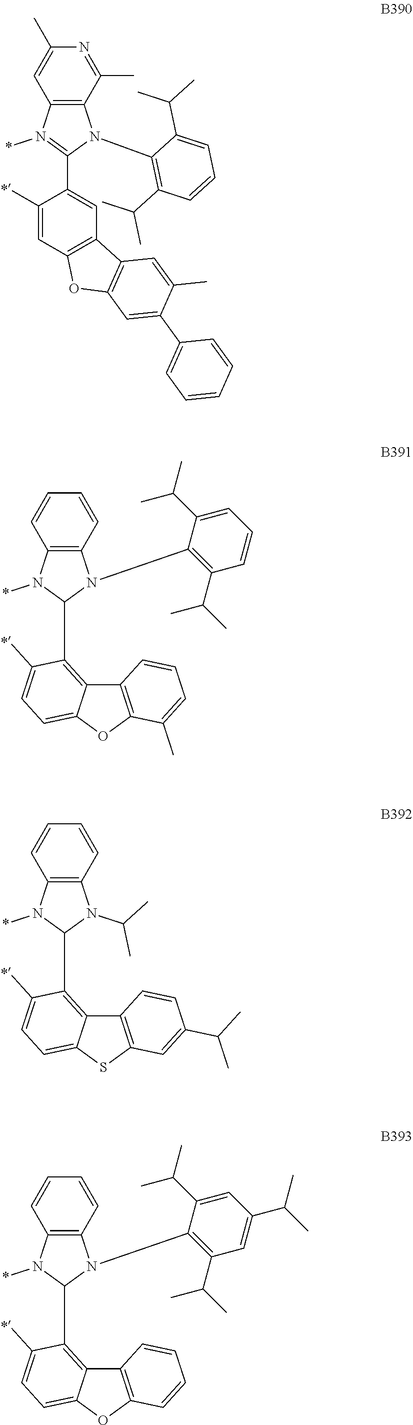

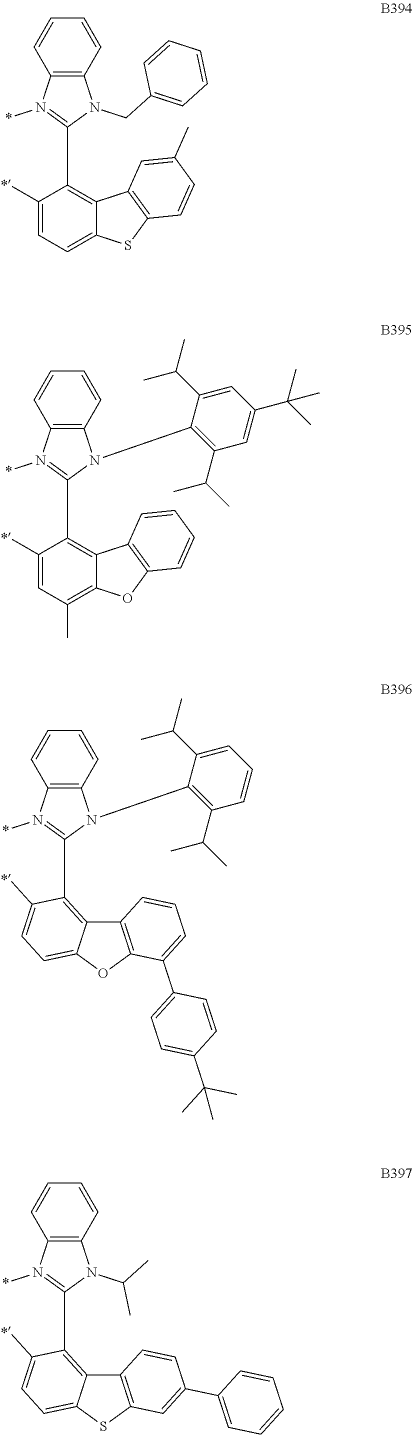

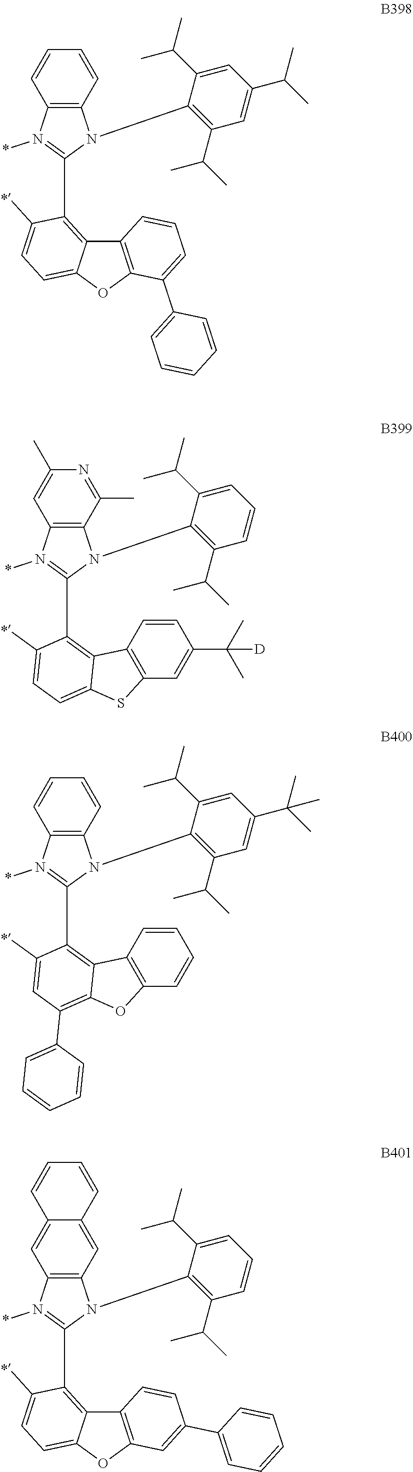

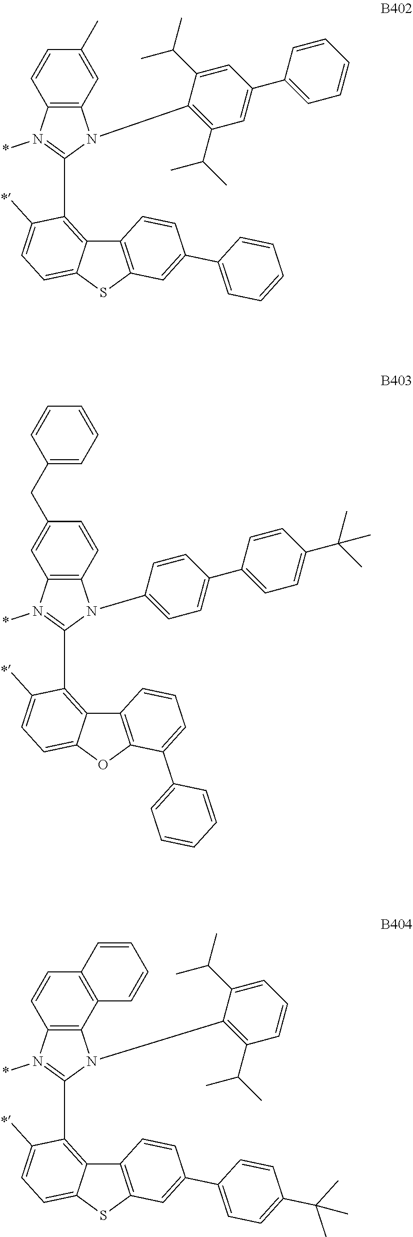

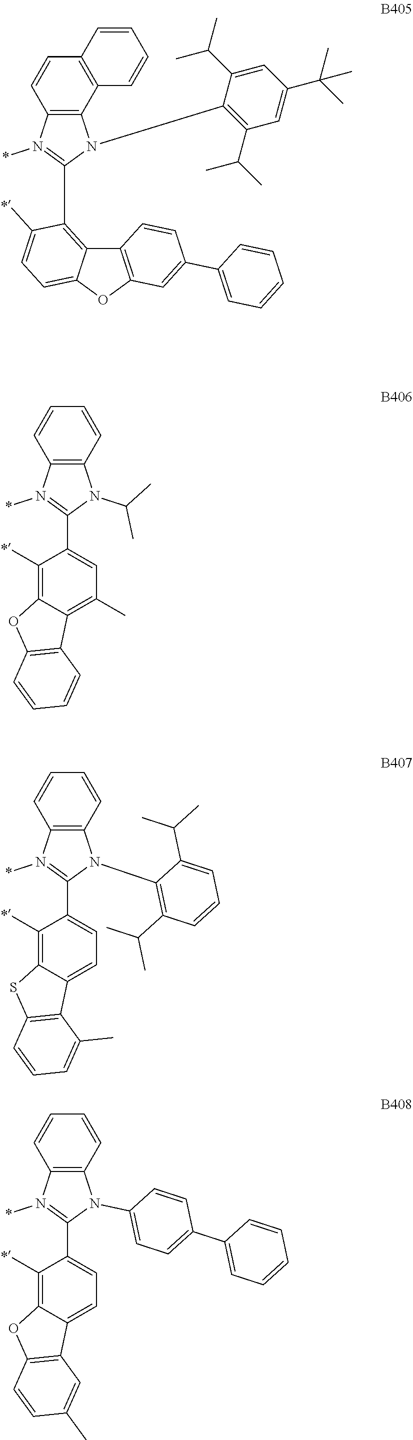

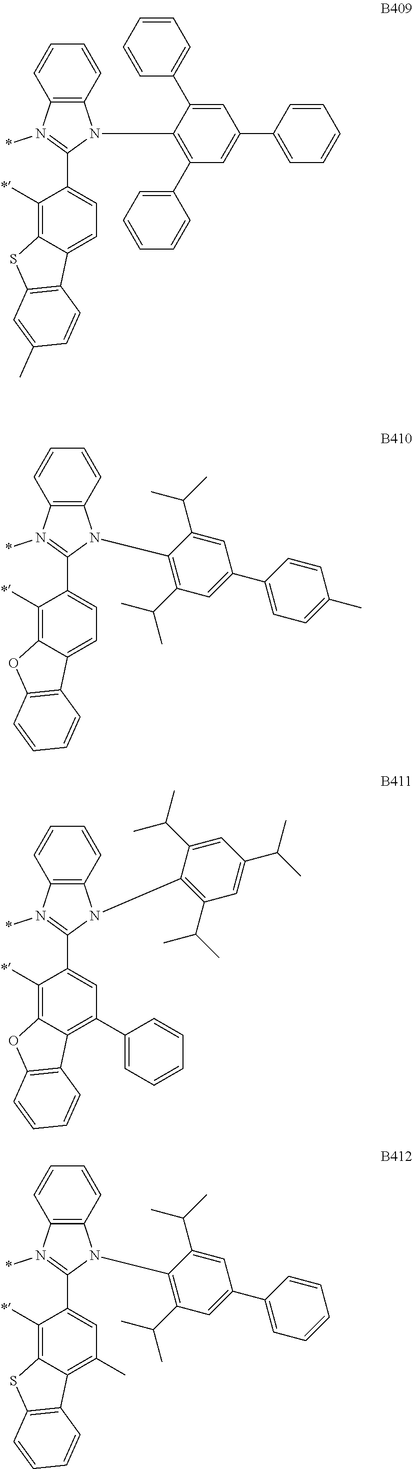

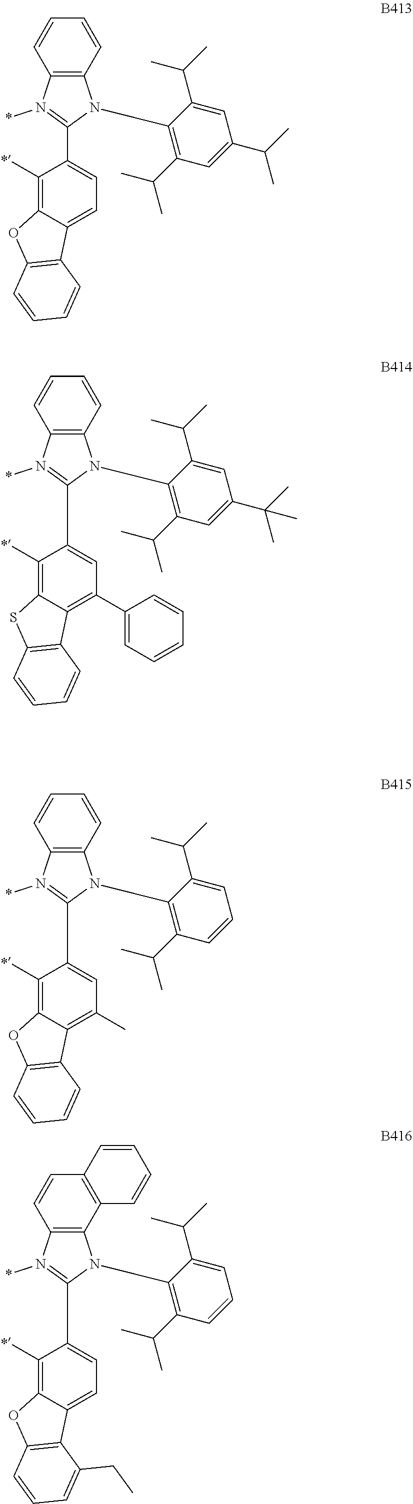

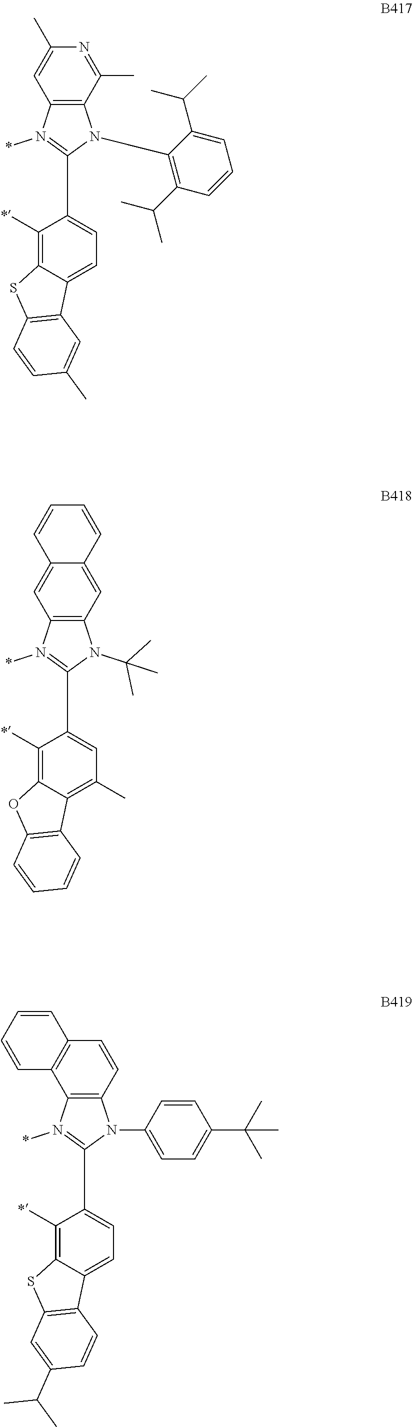

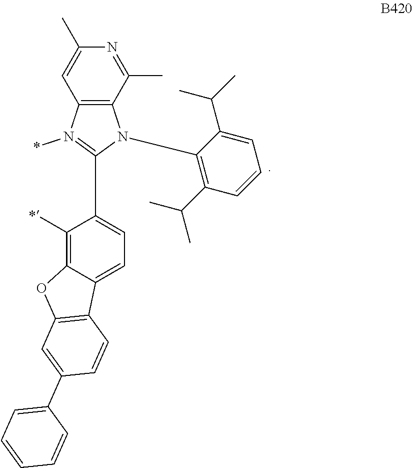

15. An organic light-emitting device comprising:
a first electrode;
a second electrode; and
an organic layer located between the first electrode and the second electrode and comprising an emission layer,
wherein the organic layer comprises the organometallic compound of claim 1.

16. The organic light-emitting device of claim 15, wherein the organometallic compound is included in the emission layer.

17. The organic light-emitting device of claim 16, wherein the emission layer further comprises a host and the amount of the host is greater than the amount of the organometallic compound.

18. The organic light-emitting device of claim 17, wherein the emission layer emits green light having a maximum emission wavelength of about 500 nm to about 600 nm.

19. The organic light-emitting device of claim 16, wherein the first electrode is an anode,
the second electrode is a cathode,
the organic layer further comprises a hole transport region located between the first electrode and the emission layer and an electron transport region located between the emission layer and the second electrode, wherein
the hole transport region comprises a hole injection layer, a hole transport layer, an electron blocking layer, a buffer layer, or a combination thereof, and
the electron transport region comprises a hole blocking layer, an electron transport layer, an electron injection layer, or a combination thereof.

20. A diagnostic composition comprising the organometallic compound of claim 1.
